(19) United States
(12) Patent Application Publication
(10) Pub. No.: US 2025/0114466 A1
(43) Pub. Date: Apr. 10, 2025

(54) ANTIFOLATE LINKER-DRUGS AND ANTIBODY-DRUG CONJUGATES

(71) Applicant: BYONDIS B.V., Nijmegen (NL)

(72) Inventors: Ronald Christiaan ELGERSMA, Nijmegen (NL); Tijl HUIJBREGTS, Nijmegen (NL); Dennis Christian Johannes WAALBOER, Nijmegen (NL); Johannes Albertus Frederikus JOOSTEN, Nijmegen (NL); Christina Adriana BEUCKENS-SCHORTINGHUIS, Nijmegen (NL)

(21) Appl. No.: 18/725,507

(22) PCT Filed: Dec. 22, 2022
(86) PCT No.: PCT/EP2022/087424

§ 371 Date: Jun. 28, 2024

(30) Foreign Application Priority Data
Dec. 30, 2021 (EP) 21218322.2

(51) Int. Cl. A61K 47/68; A61P 35/00

(52) U.S. Cl. CPC A61K 47/6803; A61K 47/6855; A61K 47/6889; A61P 35/00

(57) ABSTRACT

The present invention relates to novel antifolate linker-drugs, conjugates comprising such antifolate linker-drugs, and the use thereof in the treatment of diseases, such as cancer, autoimmune and infectious diseases, optionally in combination with other therapeutic agents.

### FIELD OF THE INVENTION

The present invention relates to novel antifolate linker-drugs, conjugates comprising such antifolate linker-drugs, and the use thereof in the treatment of diseases, such as cancer, autoimmune and infectious diseases, optionally in combination with other therapeutic agents.

### BACKGROUND OF THE PRESENT INVENTION

Antifolates are a class of antimetabolite compounds that antagonise the actions of folic acid (vitamin B9).

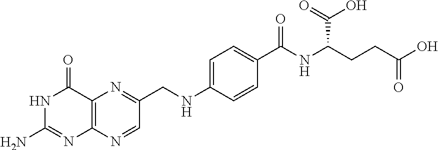 [SMILES: Nc1nc2ncc(CNc3ccc(C(=O)N[C@@H](CCC(=O)O)C(=O)O)cc3)nc2c(=O)[nH]1]

### Molecular Structure of Folic Acid

Folic acid acts as a cofactor to various methyltransferases involved in serine, methionine, thymidine, and purine biosynthesis. Consequently, antifolates inhibit cell division, DNA and RNA synthesis and repair, and protein synthesis. The majority of antifolates work by inhibiting dihydrofolate reductase (DHFR).

The antifolates proguanil, pyrimethamine, and trimethoprim selectively inhibit the actions of folic acid in microbial organisms such as bacteria, protozoa, and fungi. Other antifolates, such as methotrexate, pemetrexed, pralatrexate, and talotrexin, are used to treat some types of cancer and/or inflammatory conditions, such as the autoimmune disease rheumatoid arthritis.

Methotrexate, formerly known as amethopterin, is the oldest and most well-known folic acid analog. It was discovered in the 1950s and is a chemotherapeutic agent and immune system suppressant. It was originally developed for chemotherapy, either alone or in combination with other agents, and is still used in the treatment of e.g., bladder cancer, breast cancer, head and neck cancer, leukemia, lung cancer, lymphoma, gestational trophoblastic disease, and osteosarcoma. Methotrexate is also used as a disease-modifying treatment for some autoimmune diseases, including rheumatoid arthritis, juvenile dermatomyositis, psoriasis, psoriatic arthritis, lupus, sarcoidosis, Crohn's disease, eczema, and vasculitis. Further uses include the treatment of ectopic pregnancy and induction of medical abortions.

Pemetrexed is used in the treatment of various cancer indications, including mesothelioma, lung cancer, and head and neck cancer. It is chemically similar to folic acid and works by inhibiting DHFR, thymidylate synthase (TS), and glycinamide ribonucleotide formyltransferase (GARFT), three enzymes used in purine and pyrimidine synthesis. By inhibiting the formation of precursor purine and pyrimidine nucleotides, pemetrexed prevents the formation of DNA and RNA, which are required for the growth and survival of both normal cells and tumor cells.

Pralatrexate is approved for the treatment of peripheral T-cell lymphoma. It was designed to have increased affinity for reduced folate carrier 1 (RFC-1) as well as folylpolyglutamate synthetase, resulting in increased intracellular uptake and cytotoxic metabolites, respectively.

Talotrexin is an antimetabolite analog of aminopterin, the 4-amino derivative of folic acid and a synthetic derivative of pterin. Talotrexin exhibits antineoplastic activity and binds to and inhibits the function of DHFR. Hydrosoluble talotrexin is actively transported into cells by the reduced folate carrier (RFC) and, therefore, is unlikely to be associated with P-glycoprotein-mediated multidrug resistance.

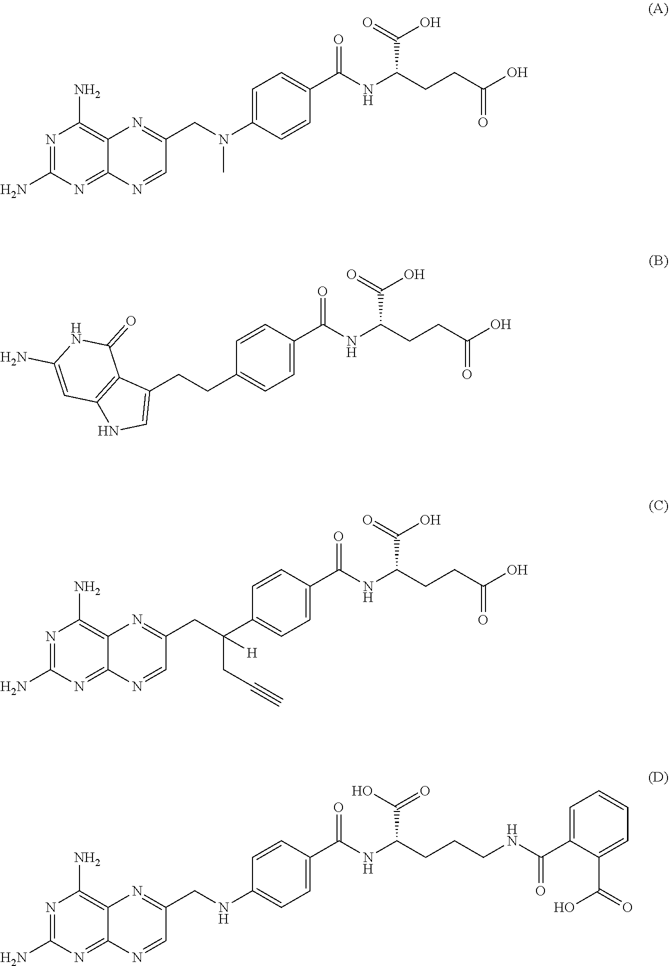 [SMILES: CN(Cc1cnc2nc(N)nc(N)c2n1)c1ccc(C(=O)N[C@@H](CCC(=O)O)C(=O)O)cc1 ; Nc1cc2[nH]cc(CCc3ccc(C(=O)N[C@@H](CCC(=O)O)C(=O)O)cc3)c2c(=O)[nH]1 ; C#CCC(Cc1cnc2nc(N)nc(N)c2n1)c1ccc(C(=O)N[C@@H](CCC(=O)O)C(=O)O)cc1 ; Nc1nc(N)c2nc(CNc3ccc(C(=O)N[C@@H](CCCNC(=O)c4ccccc4C(=O)O)C(=O)O)cc3)cnc2n1]

### Molecular Structure of (A) Methotrexate, (B) Pemetrexed, (C) Pralatrexate, and (D) Talotrexin

Antifolates act specifically during DNA and RNA synthesis, and thus are predominantly cytotoxic during the S-phase of the cell cycle. They therefore have a greater toxic effect on rapidly dividing cells (such as malignant and myeloid cells), which replicate their DNA more frequently. However, they do not only inhibit the growth and proliferation of tumor cells, but also of rapidly dividing non-cancerous cells such as bone marrow cells, and gastro-intestinal and oral mucosa cells which leads to adverse events in e.g., bone marrow, intestines, oral mucosa, skin and hair.

Systemic toxic side effects caused by a cytotoxic small molecule drug, such as an antifolate, may be reduced by conjugating such drug, via a chemical linker, to a targeting molecule, such as for example an antibody, an antigen-binding antibody fragment or a fusion protein (e.g., a receptor ligand fused to an antibody Fc). By combining these two components. i.e., the cytotoxic small molecule drug and the targeting molecule, into a single new molecular entity, the targeting molecule can specifically deliver the cytotoxic small molecule drug to target cells or tissues (where it can exert its cytotoxic activity), thereby reducing the exposure of non-target tissue to the small molecule.

In order to improve targeting and reduce toxicity of methotrexate, several antibody-drug conjugates (ADCs) of methotrexate have been prepared in the late 1980s. The drug-to-antibody ratios (DARs) of these lysine-conjugated ADCs ranged from 9.4 to 45. Although the ADC concept was successful, the obtained in vitro (Shen et al, Cancer Res. 1986, 46, 3912-3916; Umemoto et al, Int. J Cancer 1989, 43, 677-684) and in vivo (Deguchi et al, Cancer Res. 1986, 46, 3751-3755; Rowland et al, Br. J Cancer 1990, 61, 702-708) efficacy results with these ADCs were mediocre to poor. Even at a high DAR, methotrexate did not appear potent enough as an ADC toxin.

Although short tumor targeting peptide conjugates of pemetrexed have been made and tested (Miklán et al, J. Pept. Sci. 2011, 17, 805-811), this concept was not explored further. ADCs of more potent antifolates, such as pemetrexed, pralatrexate, and talotrexin, have never been reported.

Hence, there is a need for new and improved target-specific conjugates comprising an antifolate and an antibody, antigen-binding fragment or other targeting molecule, which are suitable for use in the treatment of cancer, autoimmune and infectious diseases, either alone or in combination with other therapeutic agents.

### BRIEF DESCRIPTION OF THE PRESENT INVENTION

The present invention relates to novel antifolate linker-drugs, conjugates comprising such antifolate linker-drugs, and the use thereof in the treatment of diseases, such as cancer, autoimmune and infectious diseases, optionally in combination with other therapeutic agents.

In a first aspect, the present invention relates to a linker-drug compound of formula (I)

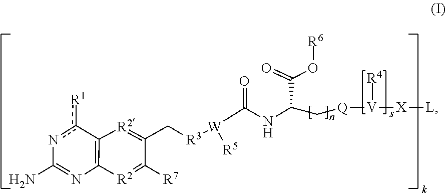

wherein
R¹ is O, NH₂ or OH;
R² and R^{2′} are independently N, CH, CMe, C-Et, C-ethyn, C-vinyl, C—CN, C—OH, C—OMe, C—SH, C—SMe, C-halogen, wherein halogen is preferably Cl, C—CHal₃, C—CHHal₂, or C—CH₂Hal, wherein Hal is halogen, preferably F;
R³ is O, S, NH, N(C₁₋₅ alkyl), N(C₂₋₄ alkenyl), N(C₂₋₄ alkynyl), N—CH₂CN, N—CH₂CH₂Hal, CH₂, CH(C₁₋₅ alkyl), CH(C₂₋₄ alkenyl), CH(C₂₋₄ alkynyl), CH(C₁₋₄ alkoxyl), CH—CH₂CN, or CH—CH₂CH₂Hal, wherein Hal is halogen, preferably F;
R⁴ is H, halogen, —COOH, OH, NH₂, —CONH₂, —CONHR, —CONR₂, C₁₋₄ alkyl, C₁₋₄ alkoxyl, benzyloxy, tetrazole, —SO₃H, —NSO₃H, —OSO₃H, —PO₃H₂, —NPO₃H₂, —OPO₃H₂, —CN, or azido, wherein R is selected from H and C₁₋₅ alkyl, or R⁴ is a carboxylic acid bioisostere selected from the group consisting of

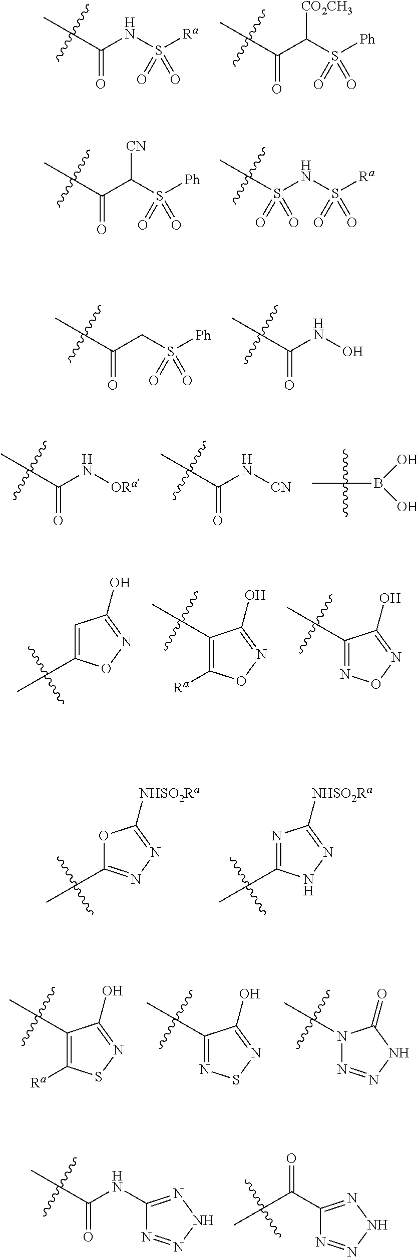

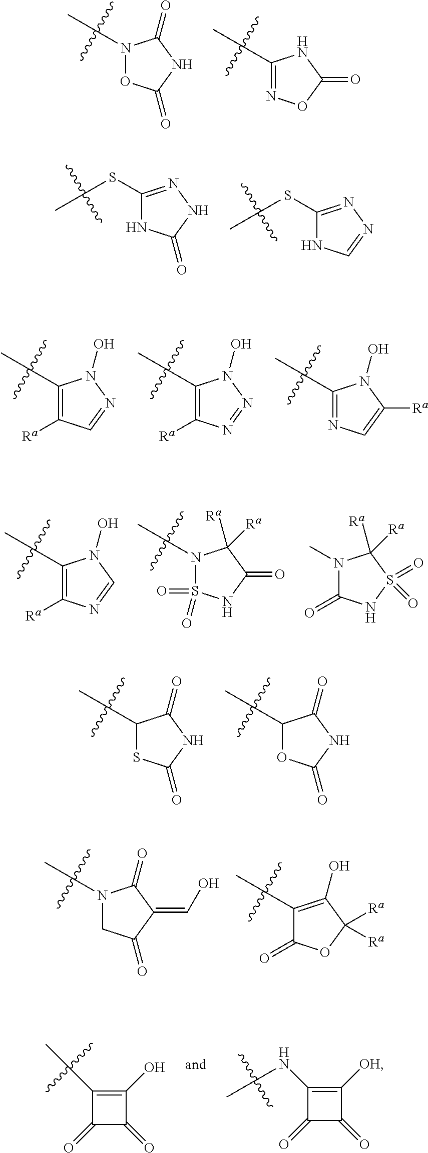

wherein R^{a}′ is selected from H, CH₂F, CHF₂, CF₃, and C₁₋₆ alkyl, each R^{a} is independently selected from H, F, CH₂F, CHF₂, CF₃, and C₁₋₆ alkyl, and two R^{a} substituents can optionally be joined forming a ring;
R⁵ is H, halogen, NH₂, OH, SH, CF₃, C₁₋₄ alkyl, C₂₋₄ alkenyl, C₂₋₄ alkynyl, C₁₋₄ alkoxyl, or C₁₋₄ alkylthio, preferably H, F, CH₃, CF₃, CH₂CH₃, CH═CH₂, CH₂CF₃, or CF₂CF₃, more preferably H or F, or R³ and R⁵ are optionally being joined by one or more bonds to form an optionally substituted carbocycle or heterocycle, preferably an optionally substituted 5- or 6-membered carbocycle or heterocycle;
R⁶ is H, C₁₋₄ alkyl, C₂₋₄ alkenyl, C₃₋₆ cycloalkyl, preferably H;
R⁷ is H or F;
n is 1, 2, 3, or 4, preferably 3;
Q is either absent, —N(R⁸)—(C═O)—, —(C═O)—N(R⁸)—, —CH₂N(R⁸)—, —N(R⁸)CH₂—, —N(R⁸)SO₂—, or —SO₂N(R⁸)—, wherein R⁸ is H, C₁₋₄ alkyl, C₁₋₄ alkenyl, or C₁₋₄ alkynyl, preferably H, or Q is an amide bond bioisostere selected from the group consisting of

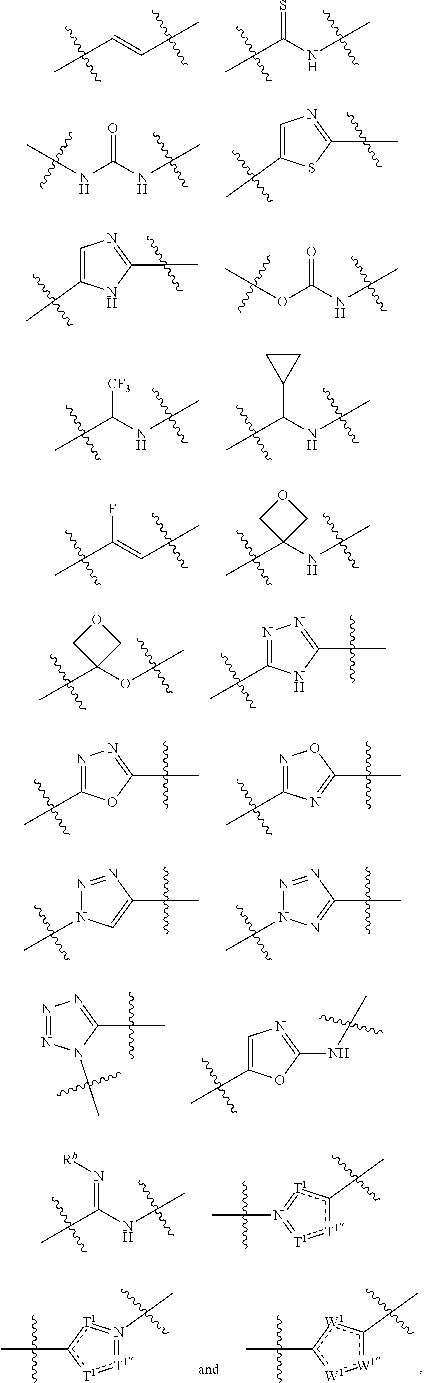

wherein R^{b} is selected from H and C₁₋₅ alkyl, T¹, T^{1′}, and T^{1″} are independently selected from CH and N, and W¹, W^{1′}, and W^{1″} are independently selected from C, CH, S, N, NH, N(C₁₋₅ alkyl), and O;
V is an aryl, heteroaryl, heterocycle, or cycloalkane that is optionally substituted with one or more R⁴-groups and is independently selected from

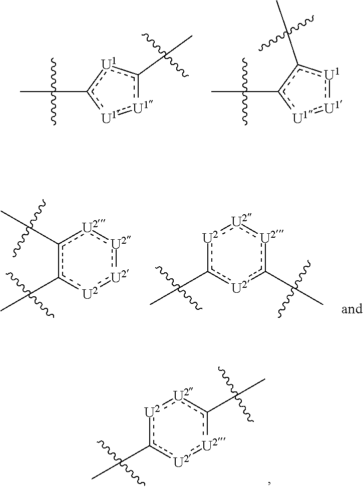

wherein U¹, U^{1′}, U^{1″}, U², U^{2′}, U^{2″}, and U^{2′″} are independently selected from C, CH, S, N, NH, N(C₁₋₅ alkyl), and O, preferably V is phenylene that is optionally substituted with one or more R⁴-groups, or V is selected from the group consisting of

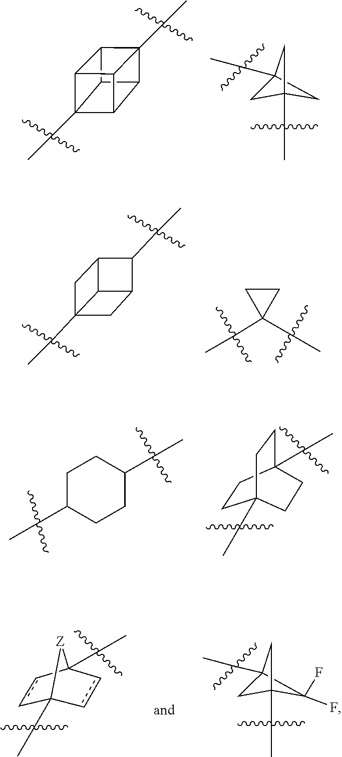

wherein Z is O, S, NH or NR^{c}, and R^{c} is selected from H and C₁₋₅ alkyl;
s is 0 or 1, preferably 1;
W is an aryl or heteroaryl that is optionally substituted with one or more R⁵-groups;
X is a connecting group selected from O, NH, S, C₁₋₅ alkylene, C₁₋₅ alkenylene, and C₁₋₅ alkynylene;
k is 1, 2, 3 or 4, preferably 1;
L is a linker moiety; and
[symbol] means that the indicated bond may be a single bond or a non-cumulated, optionally delocalized, double bond.

In a second aspect, the present invention relates to an antibody-drug conjugate of formula (III)

Ab-(L-D)_{y} (III),
wherein Ab is an antibody or an antigen-binding fragment thereof,
L-D is a linker-drug compound according to the invention;
y represents an average drug-to-antibody ratio of from 1 to 16;
and wherein the linker-drug compound according to the invention is conjugated to the antibody or antigen-binding fragment thereof, preferably through a cysteine residue of the antibody or the antigen-binding fragment.

Other aspects of the present invention include pharmaceutical compositions comprising the linker-drug compound or the antibody-drug conjugate of the invention, a process for their synthesis and their use as a medicament, particularly for the treatment of cancer, autoimmune or infectious diseases.

### BRIEF DESCRIPTION OF THE DRAWINGS

FIG. 1. In vitro efficacy of trastuzumab-XT17 antifolate ADCs versus a non-binding control ADC in HER2-positive SK-BR-3 cells.

FIG. 2. In vitro efficacy of trastuzumab-XT17 antifolate ADCs versus a non-binding control ADC in HER2-negative SW-620 cells.

FIG. 3A. In vivo efficacy of antifolate ADC1 (5 mg/kg IV) versus vehicle control in the HER2-positive BT-474 cell line xenograft in mice.

FIG. 3B. In vivo efficacy of antifolate ADC1 (5 mg/kg IV or 1.7 mg/kg Q1Wx3) versus vehicle control in the HER2-positive BT-474 cell line xenograft in mice.

FIG. 4A. Effect on body weight of antifolate ADC1 (5 mg/kg IV) versus vehicle control in the HER2-positive BT-474 cell line xenograft in mice.

FIG. 4B. Effect on body weight of antifolate ADC1 (5 mg/kg IV or 1.7 mg/kg Q1Wx3) versus vehicle control in the HER2-positive BT-474 cell line xenograft in mice.

FIG. 5. In vivo efficacy of antifolate ADC1 (3 or 10 mg/kg IV) versus vehicle control in the HER2 2+ MAXF574 patient-derived xenograft in mice.

### DETAILED DESCRIPTION OF THE PRESENT INVENTION

The inventors found that linker-drug compounds of formula (I) are particularly suitable for conjugating to an antibody, an antigen-binding fragment or another targeting molecule, such as a fusion protein (e.g., a receptor ligand fused to an antibody Fc). The small molecule drug compounds released from these linker-drug conjugates have good efficacy as antifolate compounds. They show excellent inhibitory activity of DHFR and exhibit a strong antiproliferative effect in various cancer cell lines.

### Linker-Drug Compounds

In a first aspect, the invention provides a linker-drug compound of formula (I)

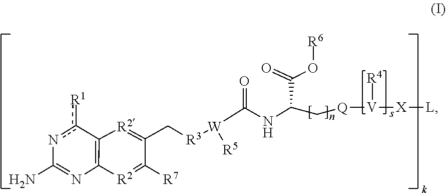

wherein
R¹ is O, NH₂ or OH;
R² and R^{2′} are independently N, CH, CMe, C-Et, C-ethyn, C-vinyl, C—CN, C—OH, C—OMe, C—SH, C—SMe, C-halogen, wherein halogen is preferably Cl, C—CHal₃, C—CHHal₂, or C—CH₂Hal, wherein Hal is halogen, preferably F;
R³ is O, S, NH, N(C₁₋₅ alkyl), N(C₂₋₄ alkenyl), N(C₂₋₄ alkynyl), N—CH₂CN, N—CH₂CH₂Hal, CH₂, CH(C₁₋₅ alkyl), CH(C₂₋₄ alkenyl), CH(C₂₋₄ alkynyl), CH(C₁₋₄ alkoxyl), CH—CH₂CN, or CH—CH₂CH₂Hal, wherein Hal is halogen, preferably F;
R⁴ is H, halogen, —COOH, OH, NH₂, —CONH₂, —CONHR, —CONR₂, C₁₋₄ alkyl, C₁₋₄ alkoxyl, benzyloxy, tetrazole, —SO₃H, —NSO₃H, —OSO₃H, —PO₃H₂, —NPO₃H₂, —OPO₃H₂, —CN, or azido, wherein R is selected from H and C₁₋₅ alkyl, or R⁴ is a carboxylic acid bioisostere;
R⁵ is H, halogen, NH₂, OH, SH, CF₃, C₁₋₄ alkyl, C₂₋₄ alkenyl, C₂₋₄ alkynyl, C₁₋₄ alkoxyl, or C₁. 4 alkylthio, preferably H, F, CH₃, CF₃, CH₂CH₃, CH═CH₂, CH₂CF₃, or CF₂CF₃, more preferably H or F, or R³ and R⁵ are optionally being joined by one or more bonds to form an optionally substituted carbocycle or heterocycle, preferably an optionally substituted 5- or 6-membered carbocycle or heterocycle;
R⁶ is H, C₁₋₄ alkyl, C₂₋₄ alkenyl, C₃₋₆ cycloalkyl, preferably H;
R⁷ is H or F;
n is 1, 2, 3, or 4, preferably 3;
Q is either absent, —N(R⁸)—(C═O)—, —(C═O)—N(R⁸)—, —CH₂N(R⁸)—, —N(R⁸)CH₂—, —N(R⁸)SO₂—, or —SO₂N(R⁸)—, wherein R⁸ is H, C₁₋₄ alkyl, C₁₋₄ alkenyl, or C₁₋₄ alkynyl, preferably H, or Q is an amide bond bioisostere;
V is an aryl, heteroaryl, heterocycle, or cycloalkane that is optionally substituted with one or more independently selected R⁴-groups and is independently selected from

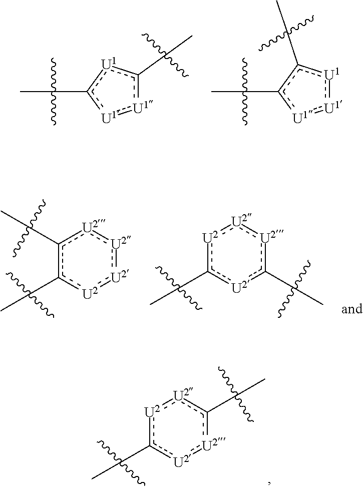

wherein U¹, U^{1′}, U^{1″}, U², U^{2′}, U^{2″}, and U^{2′″} are independently selected from C, CH, S, N, NH, N(C₁₋₅ alkyl), and O, or V is selected from the group consisting of

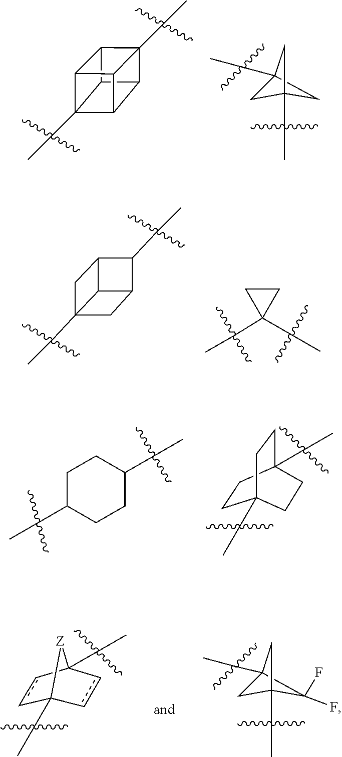

wherein Z is O, S, NH or NR^{c}, and R^{c} is selected from H and C₁₋₅ alkyl;
s is 0 or 1, preferably 1;
W is an aryl or heteroaryl that is optionally substituted with one or more independently selected R⁵-groups;
X is a connecting group selected from O, NH, S, C₁₋₅ alkylene, C₁₋₅ alkenylene, and C₁₋₅ alkynylene, preferably from O and NH, more preferably X is NH;
k is 1, 2, 3 or 4, preferably 1;
L is a linker moiety; and
[symbol] means that the indicated bond may be a single bond or a non-cumulated, optionally delocalized, double bond.

Such linker-drug compound is referred to hereinafter as a linker-drug or linker-drug compound according to the invention.

In the context of the invention, halogen is fluorine (F), chlorine (Cl), bromine (Br), or iodine (I). Preferred halogens for linker-drug compounds according to the invention are fluorine, chlorine, and bromine, more preferred halogens are fluorine or chlorine, a most preferred halogen is chlorine.

In the context of this invention, the number of carbon atoms in a moiety such as alkyl, alkenyl, alkoxyl, alkynyl, cyclyl, heterocyclyl, aryl or heteroaryl is indicated as for example C₁₋₆, in this non-limiting case indicating that from 1 to 6 carbon atoms are envisaged, such as 1, 2, 3, 4, 5, or 6 carbon atoms. Similarly, C₂₋₄ alkenyl has 2, 3, or 4 carbon atoms. The number of carbon atoms can be expressed as the total number of carbon atoms not counting further substitutions, the total number of carbon atoms, and as the number of carbon atoms that can be found in the longest continuous internal sequence of carbon atoms. Preferably, the number of carbon atoms is expressed as the total number of carbon atoms not counting further substitutions.

In the context of this invention, unsubstituted alkyl groups have the general formula CₙH₂ₙ₊₁ and may be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of suitable alkyl groups include, but are not limited to, —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, and the like. Preferred alkyl groups are linear or branched, most preferably linear. Cyclyl groups are cyclic alkyl groups; preferred cyclyl groups are cyclopropyl, cyclobutyl, and cyclopentyl. Heterocyclyl groups are cyclyl groups wherein at least one CH₂ moiety is replaced by a heteroatom. Preferred heteroatoms are S, O, and N. Preferred heterocyclyl groups are piperidinyl, oxiranyl, and oxolanyl. Preferred C₁₋₄ alkyl groups are —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, cyclopropyl, and cyclobutyl, more preferably, —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, and —C(CH₃)₃.

In the context of this invention, unsubstituted alkenyl groups have the general formula CₙH₂ₙ₋₁, and may be linear or branched. Examples of suitable alkenyl groups include, but are not limited to, ethenyl, propenyl, isopropenyl, butenyl, and pentenyl and the like. Unsubstituted alkenyl groups may also contain a cyclic moiety, and thus have the concomitant general formula CₙH₂ₙ₋₃. Preferred alkenyl groups are linear or branched, most preferably, linear.

In the context of this invention, unsubstituted alkynyl groups have the general formula CₙH₂ₙ₋₃ and may be linear or branched. Unsubstituted alkynyl groups may also contain a cyclic moiety, and thus have the concomitant general formula CₙH₂ₙ₋₅. Optionally, the alkynyl groups are substituted by one or more substituents further specified in this document. Examples of suitable alkynyl groups include, but are not limited to, ethynyl, propargyl, n-but-2-ynyl, and n-but-3-ynyl. Preferred alkyl groups are linear or branched, most preferably linear.

In the context of this invention, aryl groups are aromatic and comprise at least six carbon atoms and may include monocyclic, bicyclic and polycyclic structures. Optionally, the aryl groups may be substituted by one or more substituents further specified in this document. Examples of aryl groups include groups such as phenyl, naphthyl, anthracyl and the like. A heteroaryl group is aromatic and comprises one to four heteroatoms selected from the group consisting of S, O, and N. Due to the heteroatoms it can have a smaller ring size than six.

In the context of this invention, alkoxyl moieties are alkyl moieties that are preceded by a bridging oxygen atom. Examples of suitable alkoxyl moieties are —OCH₃, —OCH₂CH₃, —OCH₂CH₂CH₃, —OCH(CH₃)₂, —OCH(CH₃)CH₂CH₃, —OCH₂CH₂CH₂CH₃, and —OC(CH₃)₃.

In this invention, each instance of alkyl, alkenyl, alkynyl, alkoxyl, aryl, heteroaryl, cyclyl, and heterocyclyl is optionally substituted, preferably with one or more moieties selected from halogen, C₁₋₄ alkyl such as CH₃, OH, C₁₋₄ alkoxyl such as OCH₃, and ═O, wherein each instance of alkyl, alkenyl, alkynyl, and alkoxyl can be interrupted by a heteroatom such as O or S, and wherein each instance of alkyl, alkoxyl, cyclyl, and heterocyclyl is optionally unsaturated. Interruption by a heteroatom means interruption by one or more heteroatoms. In this context, preferably no more than 20, more preferably 3, 4, or 5 heteroatoms interrupt. Preferably all interrupting heteroatoms are of the same element. As a non-limiting example, a CH₂—CH₂—CH₂—CH₂—CH₃ when interrupted by heteroatoms can be CH₂—CH₂—O—CH₂—CH₂—O—CH₃.

Molecules provided in this invention can be optionally substituted. Suitable optional substitutions are replacement of —H by a halogen. Preferred halogens are F, Cl, Br, and I, most preferably F. Further suitable optional substitutions are substitutions of one or more —H by —NH₂, —OH, ═O, alkyl, alkoxyl, haloalkyl, haloalkoxyl, alkene, haloalkene, alkyn, haloalkyn, and cycloalkyl. Alkyl groups have the general formula CₙH₂ₙ₊₁ and may alternately be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of alkyl groups include methyl, ethyl, propyl, 2-propyl, t-butyl, 1-hexyl, 1-dodecyl, etc.

In the context of this invention, bioisosteres are chemical substituents or groups with similar physical or chemical properties which produce broadly similar biological properties to another chemical compound. The purpose of exchanging one bioisostere for another is to enhance the desired biological or physical properties of a compound without making significant changes in chemical structure. The general concept of bioisosteres is described in e.g. Meanwell, J Med. Chem. 2011, 54, 2529-2591 and Patani and LaVoie, Chem. Rev. 1996, 96, 3147-3176.

Carboxylic acid bioisosteres are described in e.g. Ballatore et al, ChemMedChem. 2013, 8, 385-395 and Lassalas et al, J Med. Chem. 2016, 59, 3183-3203.

Preferred examples of carboxylic acid bioisosteres are

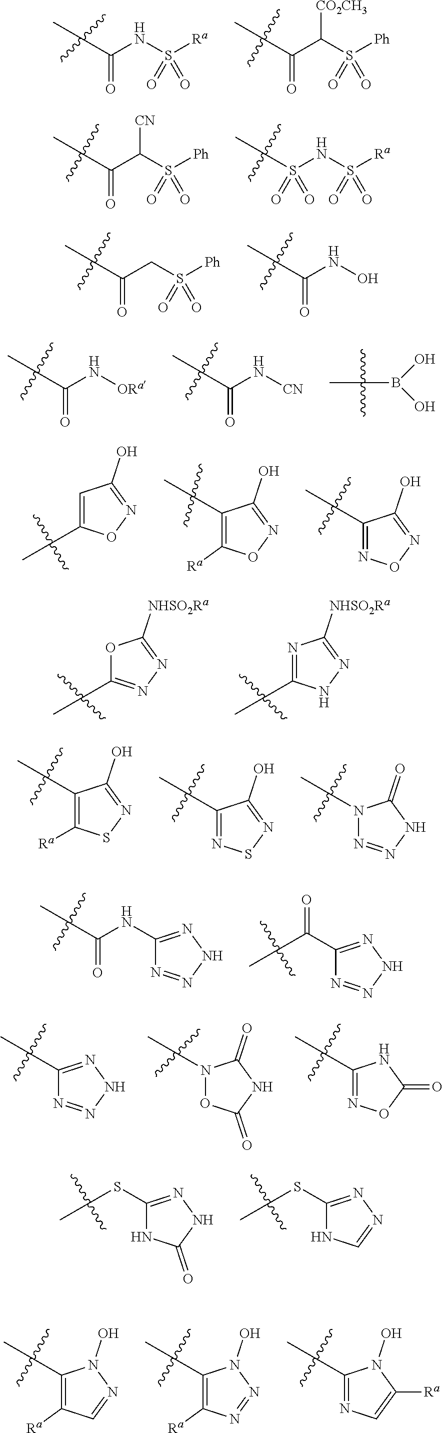

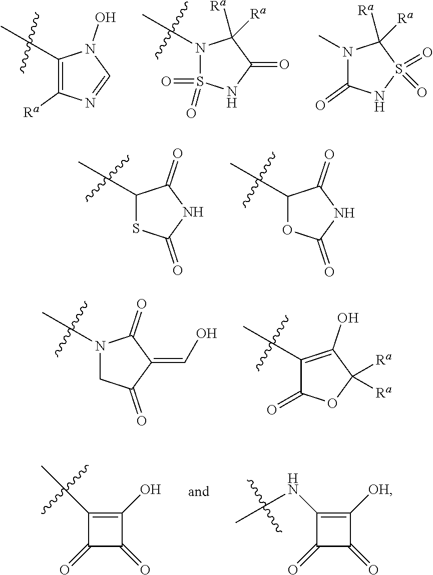

wherein R^{a}′ is selected from H, CH₂F, CHF₂, CF₃, and C₁₋₆ alkyl, each R^{a} is independently selected from H, F, CH₂F, CHF₂, CF₃, and C₁₋₆ alkyl, and two R^{a} substituents can optionally be joined forming a ring;

Amide bond bioisosteres are described in e.g. Kumari et al, J. Med. Chem. 2020, 63, 12290-12358 and Recnik et al, Molecules 2020, 25, 3576.

Preferred examples of amide bond bioisosteres are

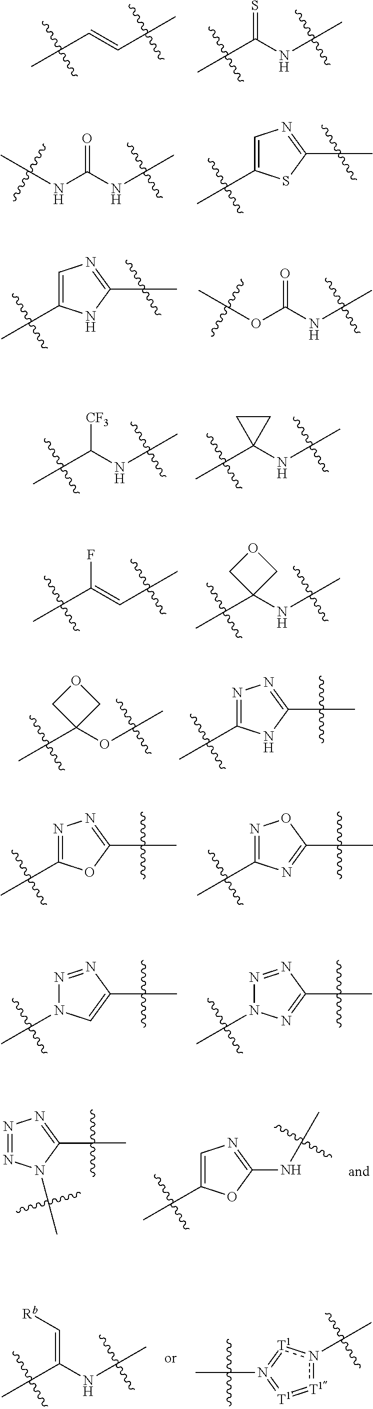

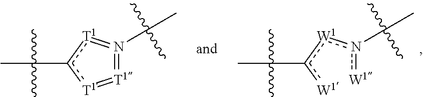

wherein R^{b} is selected from H and C₁₋₅ alkyl, T¹, T^{1′}, and T^{1″} are independently selected from CH and N, and W¹, W^{1′}, and W^{1″} are independently selected from C, CH, S, N, NH, N(C₁₋₅ alkyl), and O.

Tse et al (J. Med. Chem. 2020, 63, 11585-11601), Mykhailiuk (Org. Biomol. Chem., 2019, 17, 2839-2849), Qiao et al (Bioorg. Med. Chem. Lett. 2008, 18, 4118-4123), and Stepan et al (J. Med. Chem. 2012, 55, 3414-3424) describe various nonclassical and/or saturated phenyl bioisosteres.

Examples of such phenyl bioisosteres are

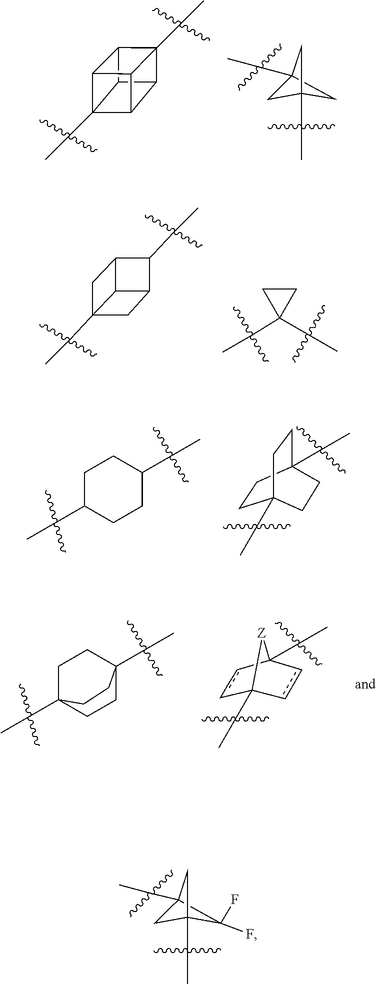

wherein Z is O, S, NH or NR^{c}, and R^{c} is selected from H and C₁₋₅ alkyl.

As described hereinabove, W is an aryl or heteroaryl that is optionally substituted with one or more independently selected R⁵-groups.

In one specific embodiment, W is

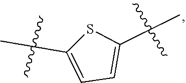 [SMILES: *c1ccc(*)s1]

and the linker-drug compound is of formula (Ia)

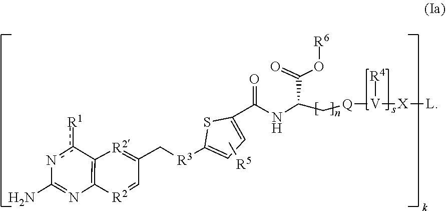

In one embodiment, the linker-drug compound is of formula (Ia), R² and R^{2′} are N, and R⁵ is H.

In another embodiment, the linker-drug compound is of formula (Ia), R² and R^{2′} are independently CH or C-halogen, and R⁵ is H. C-halogen is preferably C—Cl.

In yet another embodiment, the linker-drug compound is of formula (Ia), R³ and R⁵ are optionally being joined by one or more bonds to form an optionally substituted carbocycle or heterocycle, preferably an optionally substituted 5- or 6-membered carbocycle or heterocycle.

In another specific embodiment, W is

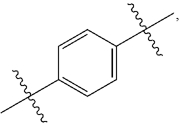 [SMILES: *c1ccc(*)cc1]

and the linker-drug compound is of formula (Ib)

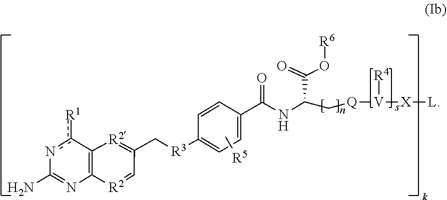

In one embodiment, the linker-drug compound is of formula (Ib), R² and R^{2′} are independently N, CH or CMe, R³ is NH, N(C₁₋₅ alkyl), CH₂, CH(C₁₋₅ alkyl), CH(C₂₋₄ alkenyl), CH(C₂₋₄ alkynyl), or CH(C₁₋₄ alkoxyl), and R⁵ is H, halogen, CF₃, C₁₋₄ alkyl, C₂₋₄ alkenyl, C₂₋₄ alkynyl, C₁₋₄ alkoxyl, or C₁₋₄ alkylthio, preferably H, F, CH₃, CF₃, CH₂CH₃, CH═CH₂, CH₂CF₃, or CF₂CF₃, more preferably H or F.

In another embodiment, the linker-drug compound is of formula (Ib), R² and R^{2′} are independently N, CH or CMe, and R³ is O, S, N(C₂₋₄ alkenyl), N(C₂₋₄ alkynyl), N—CH₂CN, N—CH₂CH₂Hal, CH—CH₂CN, or CH—CH₂CH₂Hal, wherein Hal is halogen, preferably F.

In yet another embodiment, the linker-drug compound is of formula (Ib), R² and R^{2′} are independently N, CH or CMe, and R⁵ is NH₂, OH, or R³ and R⁵ are optionally being joined by one or more bonds to form an optionally substituted carbocycle or heterocycle, preferably an optionally substituted 5- or 6-membered carbocycle or heterocycle; In yet another embodiment, the linker-drug compound is of formula (Ib), R² and R^{2′} are independently CH or C-halogen. C-halogen is preferably C—Cl.

As described hereinabove, V is an aryl, heteroaryl, heterocycle, or cycloalkane that is optionally substituted with one or more independently selected R⁴-groups and is independently selected from

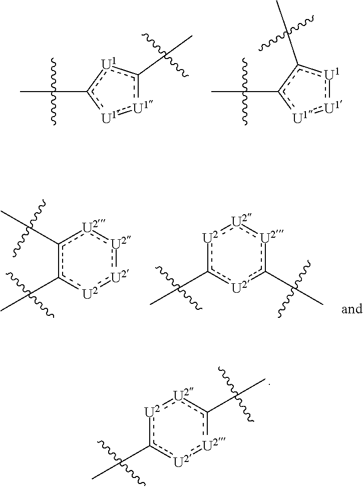

Preferably, V is selected from

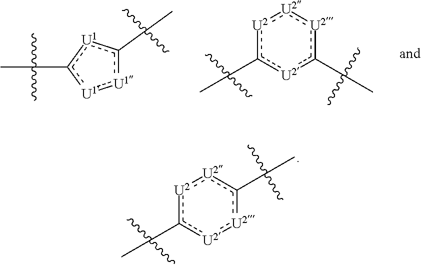

More preferably, V is selected from

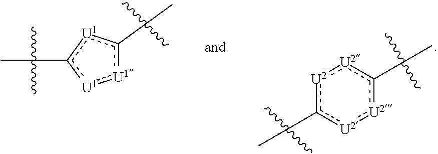

In specific embodiments, V is

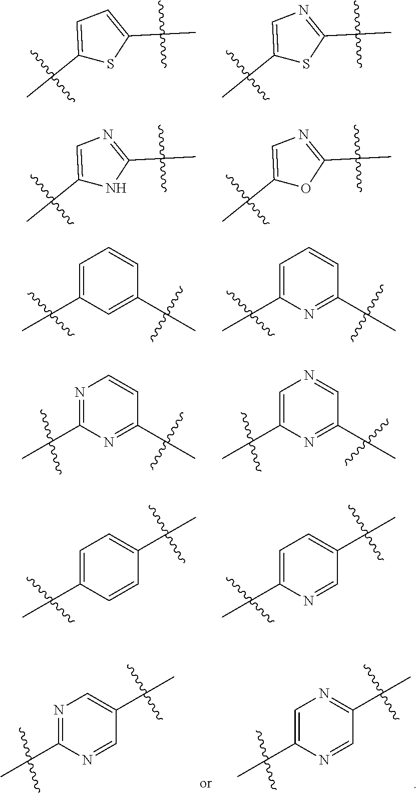

Preferably, V is

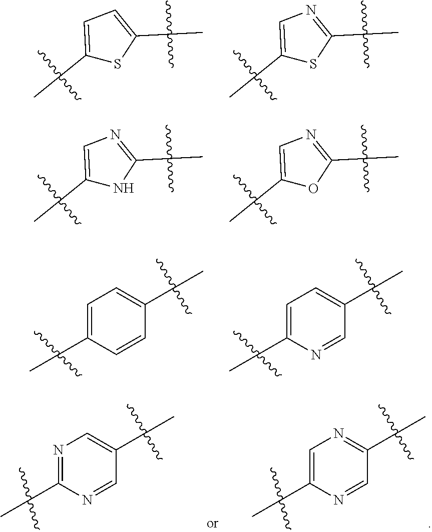

More preferably, V is a para-phenylene group as shown in the formula

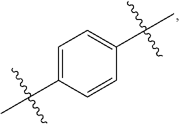 [SMILES: *c1ccc(*)cc1]

which may be substituted with one or more R⁴-groups.

In one particular embodiment, the invention provides a linker-drug compound of formula (Ia-1)

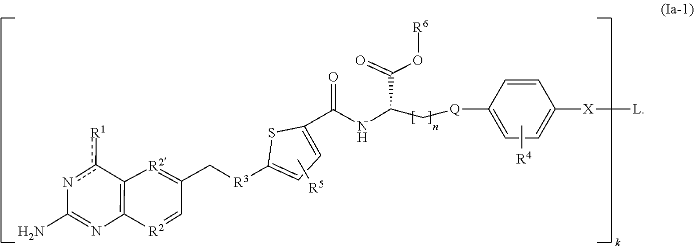

In one embodiment, the linker-drug compound is of formula (Ia-1), R² and R^{2′} are N, and R⁵ is H.

In another embodiment, the linker-drug compound is of formula (Ia-1), R² and R^{2′} are independently CH or C-halogen, and R⁵ is H. C-halogen is preferably C—Cl.

In yet another embodiment, the linker-drug compound is of formula (Ia-1), R³ and R⁵ are optionally being joined by one or more bonds to form an optionally substituted carbocycle or heterocycle, preferably an optionally substituted 5- or 6-membered carbocycle or heterocycle.

In a second particular embodiment, the invention provides a linker-drug compound of formula (Ib-1)

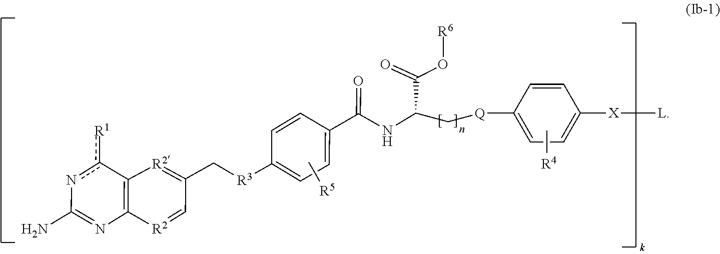

In one embodiment, the linker-drug compound is of formula (Ib-1), R² and R^{2′} are independently N, CH or CMe, R³ is NH, N(C₁₋₅ alkyl), CH₂, CH(C₁₋₅ alkyl), CH(C₂₋₄ alkenyl), CH(C₂₋₄ alkynyl), or CH(C₁₋₄ alkoxyl), and R⁵ is H, halogen, CF₃, C₁₋₄ alkyl, C₂₋₄ alkenyl, C₂₋₄ alkynyl, C₁₋₄ alkoxyl, or C₁₋₄ alkylthio, preferably H, F, CH₃, CF₃, CH₂CH₃, CH═CH₂, CH₂CF₃, or CF₂CF₃, more preferably H or F.

In another embodiment, the linker-drug compound is of formula (Ib-1), R² and R^{2′} are independently N, CH or CMe, and R³ is O, S, N(C₂₋₄ alkenyl), N(C₂₋₄ alkynyl), N—CH₂CN, N—CH₂CH₂Hal, CH—CH₂CN, or CH—CH₂CH₂Hal, wherein Hal is halogen, preferably F.

In yet another embodiment, the linker-drug compound is of formula (Ib-1), R² and R^{2′} are independently N, CH or CMe, and R⁵ is NH₂, OH, or R³ and R⁵ are optionally being joined by one or more bonds to form an optionally substituted carbocycle or heterocycle, preferably an optionally substituted 5- or 6-membered carbocycle or heterocycle; In yet another embodiment, the linker-drug compound is of formula (Ib-1), R² and R^{2′} are independently CH or C-halogen. C-halogen is preferably C—Cl.

In one particular embodiment, the invention provides a linker-drug compound of formula (Ia-2)

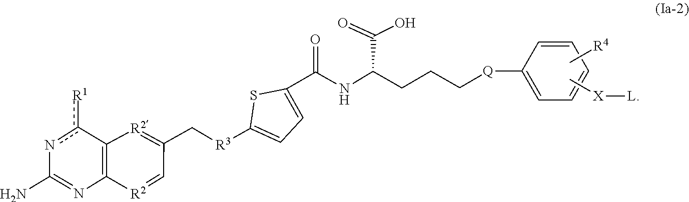

In a second particular embodiment, the invention provides a linker-drug compound of formula (Ib-2)

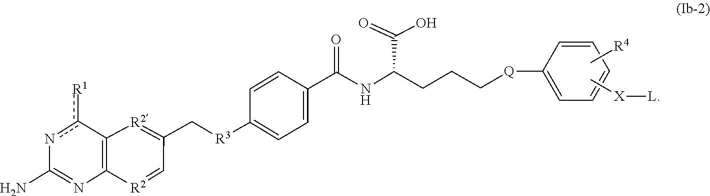

Preferably, the invention provides a linker-drug compound of formula (Ia-2) or (Ib-2),
wherein
R¹ is O, NH₂ or OH, preferably R¹ is NH₂;
R² and R^{2′} are independently N, CH, CMe, or C-halogen, preferably R² and R^{2′} are N, or R² is CH and R^{2′} is CH or C-halogen, more preferably, R^{2′} is C—Cl;
R³ is O, S, NH, N(C₁₋₅ alkyl), N(C₂₋₄ alkenyl), N(C₂₋₄ alkynyl), CH₂ or CH(C₁₋₅ alkyl), preferably R³ is O, S, NH, N(CH₃), N(CH₂—C≡CH), or CH₂;
R⁴ is H, halogen, —COOH, OH, NH₂, —CONH₂, —CONHR, —CONR₂, C₁₋₄ alkyl, C₁₋₄ alkoxyl, tetrazole, —SO₃H, —NSO₃H, —OSO₃H, —PO₃H₂, —NPO₃H₂, —OPO₃H₂, —CN, or azido, wherein R is selected from H and C₁₋₅ alkyl, preferably R⁴ is —COOH or tetrazole;
Q is either absent, —N(R⁸)—(C═O)—, —(C═O)—N(R⁸)—, —CH₂N(R⁸)—, —N(R⁸)CH₂—, —N(R⁸)SO₂—, or —SO₂N(R⁸)—, wherein R⁸ is H, C₁₋₄ alkyl, C₁₋₄ alkenyl, or C₁₋₄ alkynyl, preferably H, or Q is an amide bond bioisostere, preferably Q is —N(R⁸)—(C═O)— or —(C═O)—N(R⁸)—, more preferably —N(R⁸)—(C═O)—;
X is a connecting group selected from O, NH, S, C₁₋₅ alkylene, C₁₋₅ alkenylene, and C₁₋₅ alkynylene, preferably X is NH;
L is a linker moiety; and
[symbol] means that the indicated bond may be a single bond or anon-cumulated, optionally delocalized, double bond.

In a more specific embodiment, the invention provides a linker-drug compound of formula (Ia-2) or (Ib-2), wherein R¹ is NH₂, and R² and R^{2′} are N of formula

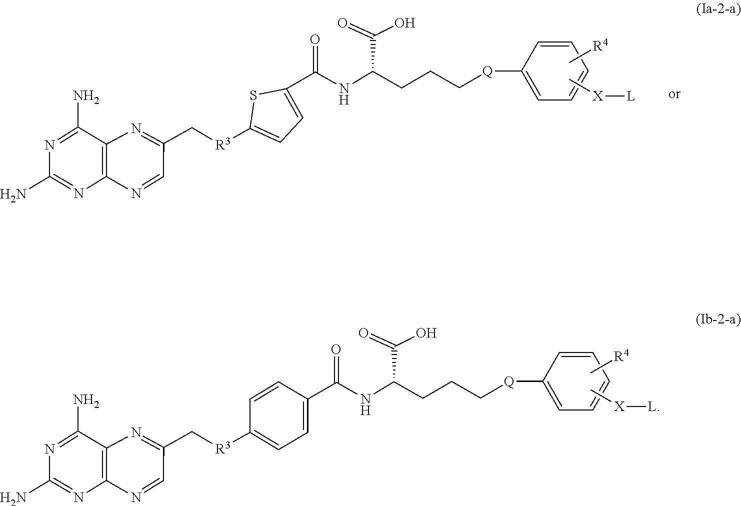

In another more specific embodiment, the invention provides a linker-drug compound of formula (Ia-2) or (Ib-2), wherein R¹ is NH₂, and R² and R^{2′} are CH of formula

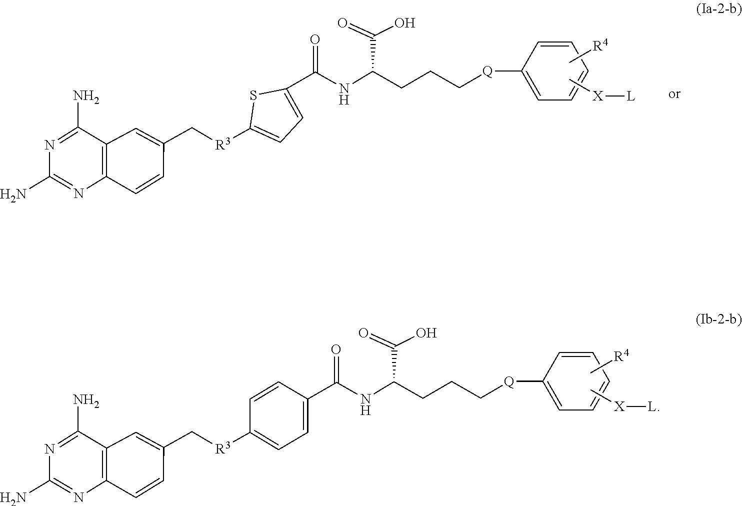

In yet another more specific embodiment, the invention provides a linker-drug compound of formula (Ia-2) or (Ib-2), wherein R¹ is NH₂, R² is CH, and R^{2′} is C-halogen of formula

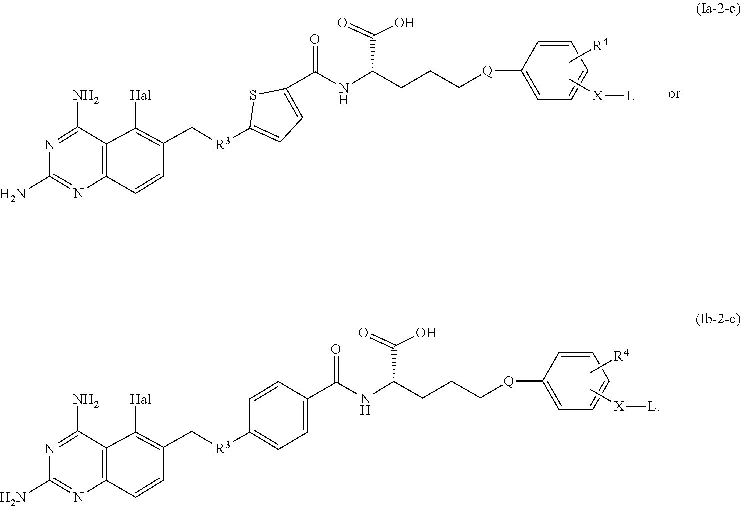

Even more specifically, the invention provides a linker-drug compound of formula (Ia-2) or (Ib-2), wherein R¹ is NH₂, R² is CH, and R^{2′} is C—Cl of formula

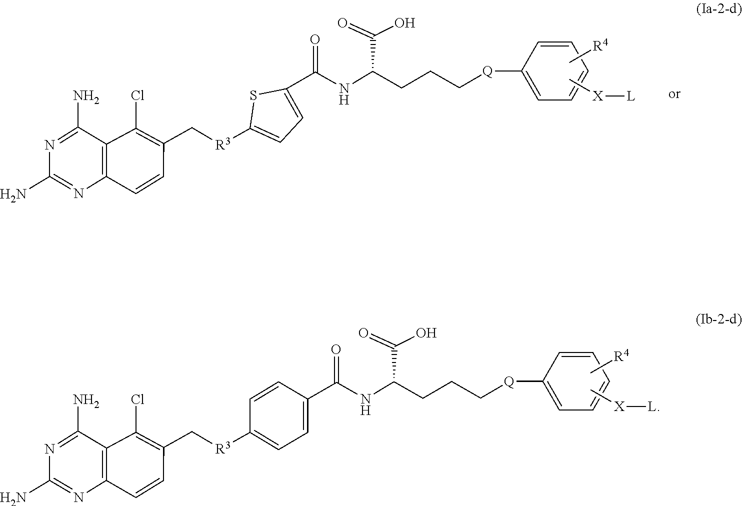

In a specific embodiment, R³ is NH, N(CH₃), or CH₂.

In another specific embodiment, R⁴ is —COOH and Q is —NH—(C═O)—.

In another specific embodiment, R⁴ is —COOH and Q is triazole.

In another specific embodiment, R⁴ is tetrazole and Q is —NH—(C═O)—.

In another specific embodiment, R⁴ is —SO₃H and Q is —NH—(C═O)—.

In one particular embodiment, the invention provides a linker-drug compound of formula (Ia-3)

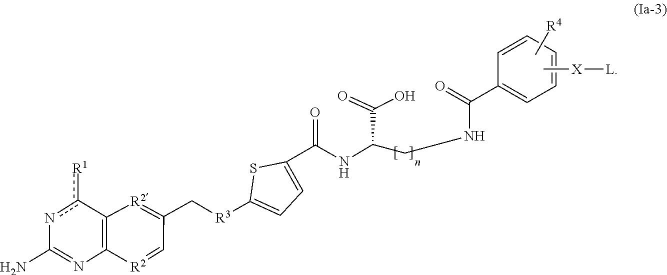

In a second particular embodiment, the invention provides a linker-drug compound of formula (Ib-3)

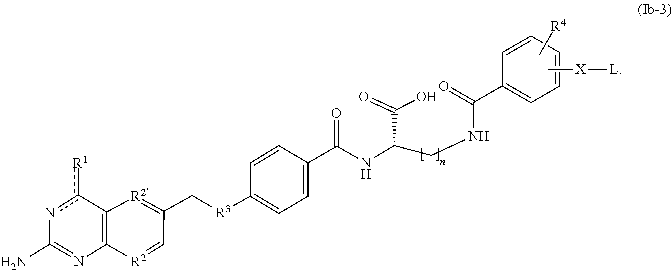

Preferably, the invention provides a linker-drug compound of formula (Ia-3) or (Ib-3),
wherein
R¹ is O, NH₂ or OH, preferably R¹ is NH₂;
R² and R^{2′} are independently N, CH, CMe, or C-halogen, preferably R² and R^{2′} are N, or R² is CH and R^{2′} is CH or C-halogen, more preferably, R^{2′} is C—Cl;
R³ is O, S, NH, N(C₁₋₅ alkyl), N(C₂₋₄ alkenyl), N(C₂₋₄ alkynyl), CH₂ or CH(C₁₋₅ alkyl), preferably R³ is O, S, NH, N(CH₃), N(CH₂—C≡CH), or CH₂;
R⁴ is H, halogen, —COOH, OH, NH₂, —CONH₂, —CONHR, —CONR₂, C₁₋₄ alkyl, C₁₋₄ alkoxyl, tetrazole, —SO₃H, —NSO₃H, —OSO₃H, —PO₃H₂, —NPO₃H₂, —OPO₃H₂, —CN, or azido, wherein R is selected from H and C₁₋₅ alkyl, preferably R⁴ is —COOH or tetrazole;
n is 1, 2, 3, or 4, preferably 3;
X is a connecting group selected from O, NH, S, C₁₋₅ alkylene, C₁₋₅ alkenylene, and C₁₋₅ alkynylene, preferably X is NH;
L is a linker moiety; and
[symbol] means that the indicated bond may be a single bond or a non-cumulated, optionally delocalized, double bond.

In a more specific embodiment, the invention provides a linker-drug compound of formula (Ia-3) or (Ib-3), wherein R¹ is NH₂, and R² and R^{2′} are N of formula

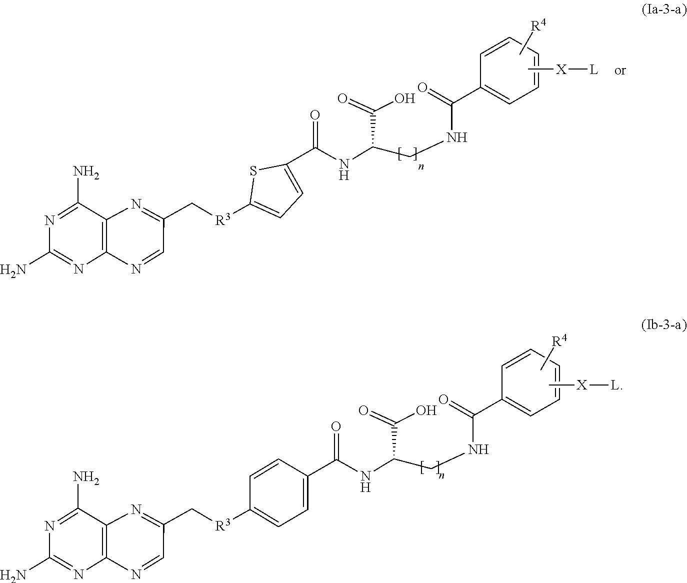

In another more specific embodiment, the invention provides a linker-drug compound of formula (Ia-3) or (Ib-3), wherein R¹ is NH₂, and R² and R^{2′} are CH of formula

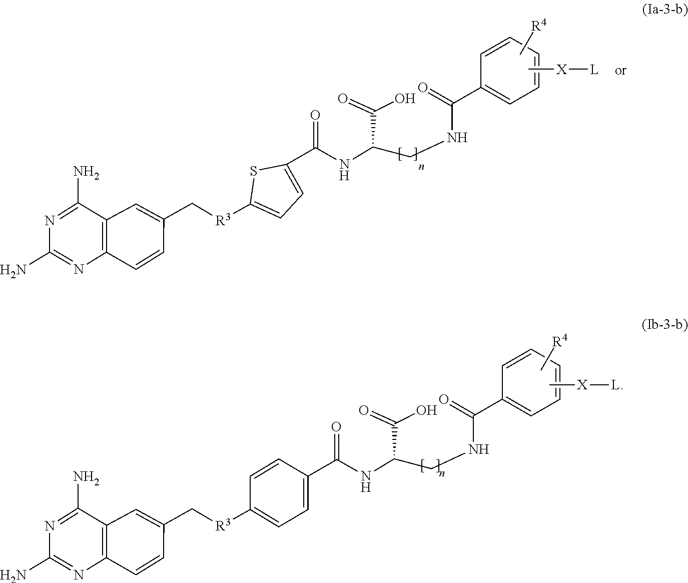

In yet another more specific embodiment, the invention provides a linker-drug compound of formula (Ia-3) or (Ib-3), wherein R¹ is NH₂, R² is CH, and R^{2′} is C-halogen of formula

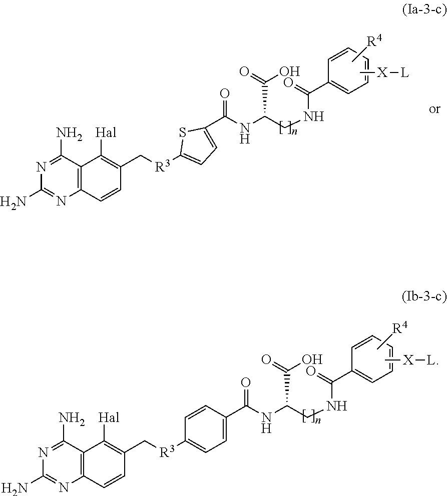

Even more specifically, the invention provides a linker-drug compound of formula (Ia-3) or (Ib-3), wherein R¹ is NH₂, R² is CH, and R^{2′} is C—Cl of formula

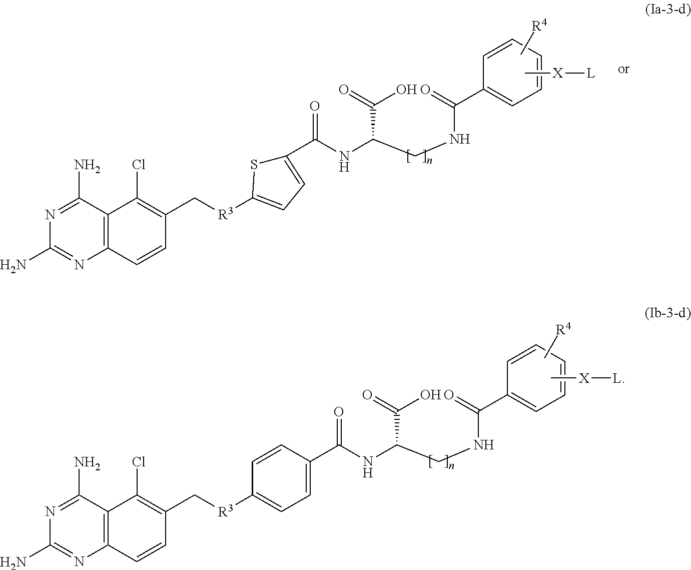

In a specific embodiment, R³ is NH, N(CH₃), or CH₂.

In another specific embodiment, R⁴ is —COOH and n is 3.

In another specific embodiment, R⁴ is tetrazole and n is 3.

In one particular embodiment, the invention provides a linker-drug compound of formula (Ia-4)

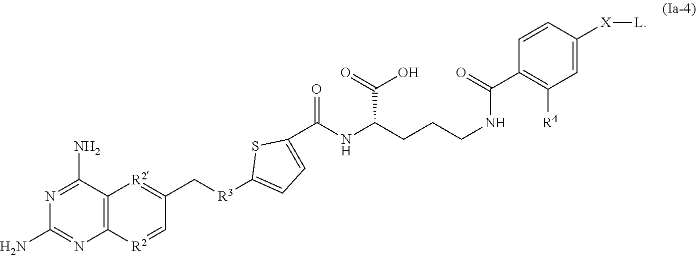

In a second particular embodiment, the invention provides a linker-drug compound of formula (Ib-4)

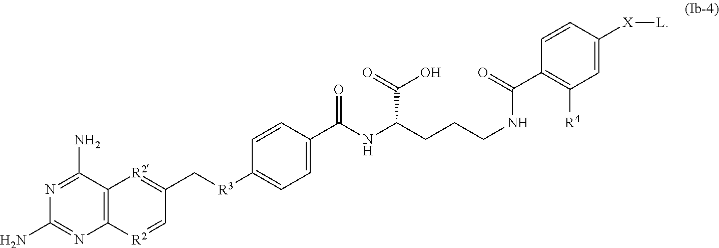

Preferably, the invention provides a linker-drug compound of formula (Ia-4) or (Ib-4),
wherein
R² and R^{2′} are independently N, CH, CMe, or C-halogen, preferably R² and R^{2′} are N, or R² is CH and R^{2′} is CH or C-halogen, more preferably, R^{2′} is C—Cl;
R³ is O, S, NH, N(C₁₋₅ alkyl), N(C₂₋₄ alkenyl), N(C₂₋₄ alkynyl), CH₂ or CH(C₁₋₅ alkyl), preferably R³ is O, S, NH, N(CH₃), N(CH₂—C≡CH), or CH₂;
R⁴ is H, halogen, —COOH, OH, NH₂, —CONH₂, —CONHR, —CONR₂, C₁₋₄ alkyl, C₁₋₄ alkoxyl, tetrazole, —SO₃H, —NSO₃H, —OSO₃H, —PO₃H₂, —NPO₃H₂, —OPO₃H₂, —CN, or azido, wherein R is selected from H and C₁₋₅ alkyl, preferably R⁴ is —COOH or tetrazole;
X is a connecting group selected from O, NH, S, C₁₋₅ alkylene, C₁₋₅ alkenylene, and C₁₋₅ alkynylene, preferably X is NH; and
L is a linker moiety.

In a more specific embodiment, the invention provides a linker-drug compound of formula (Ia-4) or (Ib-4), wherein R² and R^{2′} are N of formula

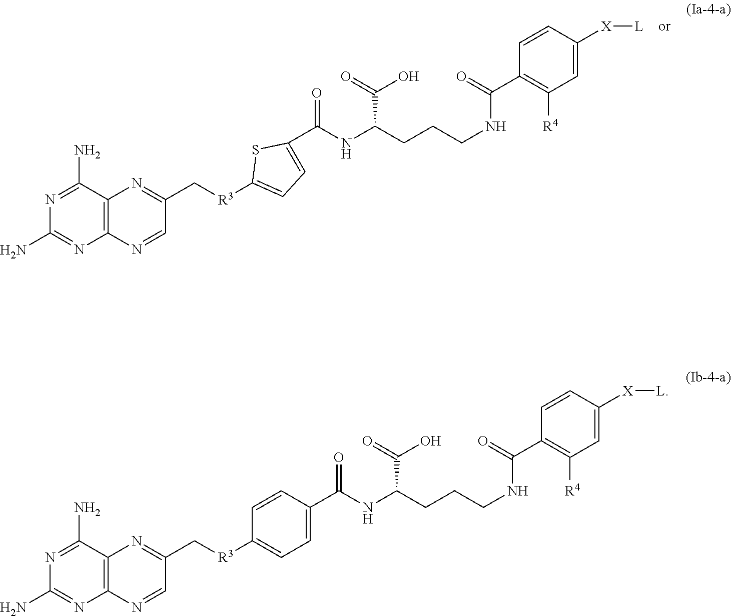

In another more specific embodiment, the invention provides a linker-drug compound of formula (Ia-4) or (Ib-4), wherein R² and R^{2′} are CH of formula

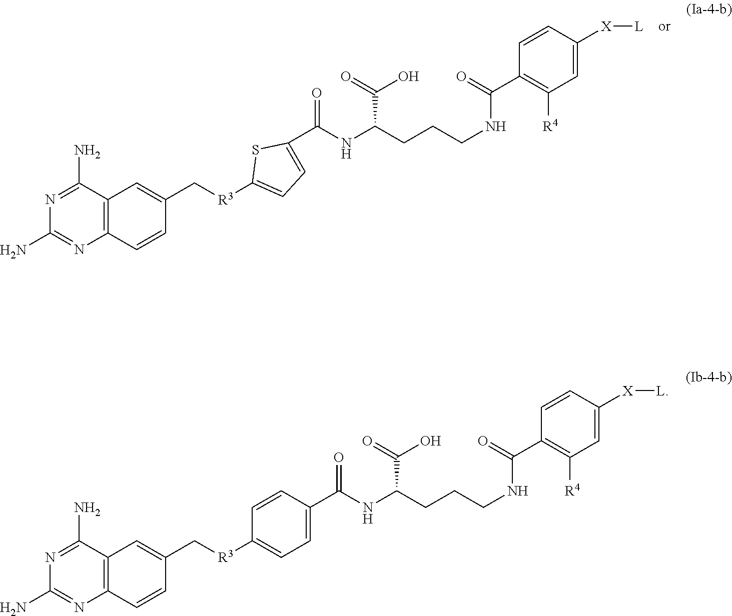

In yet another more specific embodiment, the invention provides a linker-drug compound of formula (Ia-4) or (Ib-4), wherein R² is CH and R^{2′} is C-halogen of formula

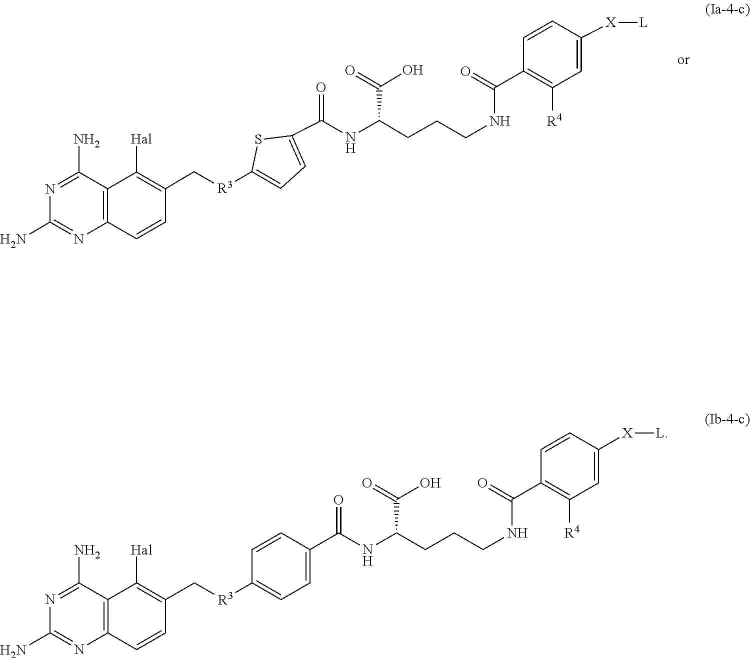

Even more specifically, the invention provides a linker-drug compound of formula (Ia-4) or (Ib-4), wherein R² is CH and R^{2′} is C—Cl of formula

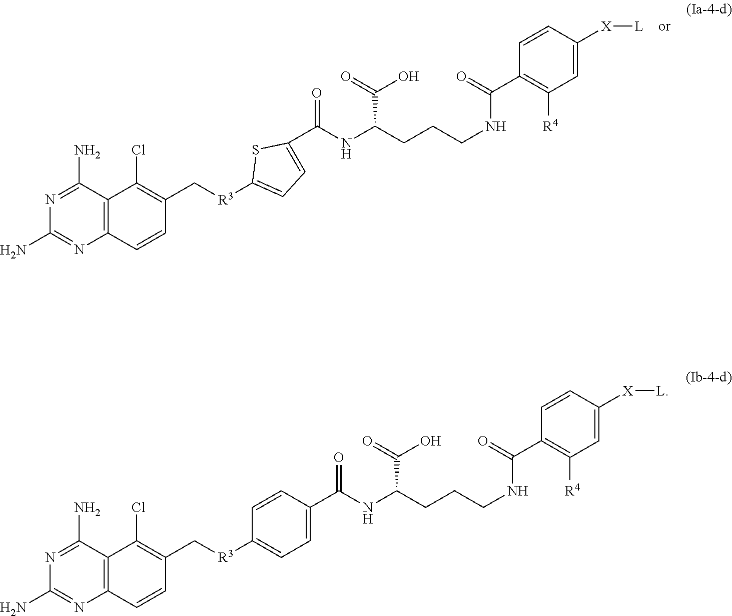

In a specific embodiment, R³ is NH, N(CH₃), or CH₂.

In another specific embodiment, R⁴ is —COOH.

In another specific embodiment, R⁴ is tetrazole.

In another specific embodiment, R⁴ is H.

In another specific embodiment, R⁴ is OH.

In another specific embodiment, R⁴ is Cl.

In another specific embodiment, R⁴ is —SO₃H.

In one particular embodiment, the invention provides a linker-drug compound of formula (Ia-5)

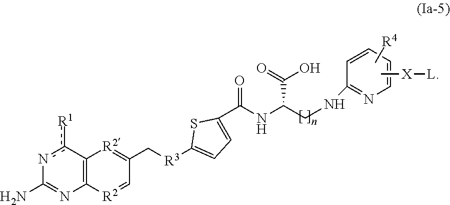

In a second particular embodiment, the invention provides a linker-drug compound of formula (Ib-5)

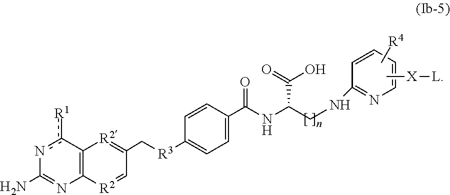

Preferably, the invention provides a linker-drug compound of formula (Ia-5) or (Ib-5),
wherein
R¹ is O, NH₂ or OH, preferably R¹ is NH₂;
R² and R^{2′} are independently N, CH, CMe, or C-halogen, preferably R² and R^{2′} are N, or R² is CH and R^{2′} is CH or C-halogen, more preferably, R^{2′} is C—Cl;
R³ is O, S, NH, N(C₁₋₅ alkyl), N(C₂₋₄ alkenyl), N(C₂₋₄ alkynyl), CH₂ or CH(C₁₋₅ alkyl), preferably R³ is O, S, NH, N(CH₃), N(CH₂—C≡CH), or CH₂;
R⁴ is H, halogen, —COOH, OH, NH₂, —CONH₂, —CONHR, —CONR₂, C₁₋₄ alkyl, C₁₋₄ alkoxyl, tetrazole, —SO₃H, —NSO₃H, —OSO₃H, —PO₃H₂, —NPO₃H₂, —OPO₃H₂, —CN, or azido, wherein R is selected from H and C₁₋₅ alkyl, preferably R⁴ is —COOH or tetrazole;
n is 1, 2, 3, or 4, preferably 4;
X is a connecting group selected from O, NH, S, C₁₋₅ alkylene, C₁₋₅ alkenylene, and C₁₋₅ alkynylene, preferably X is NH;
L is a linker moiety; and
[symbol] means that the indicated bond may be a single bond or a non-cumulated, optionally delocalized, double bond.

In a more specific embodiment, the invention provides a linker-drug compound of formula Ia-5) or (Ib-5), wherein R¹ is NH₂, and R² and R^{2′} are N of formula

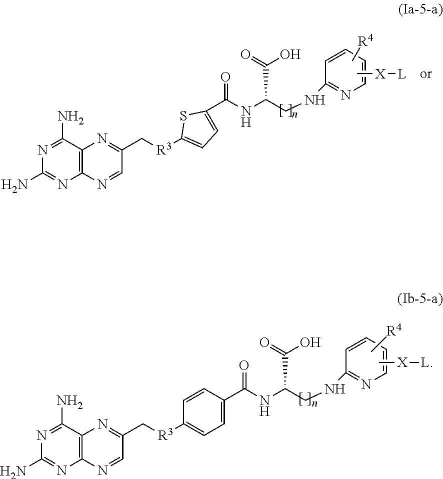

In another more specific embodiment, the invention provides a linker-drug compound of formula (Ia-5) or (Ib-5), wherein R¹ is NH₂, and R² and R^{2′} are CH of formula

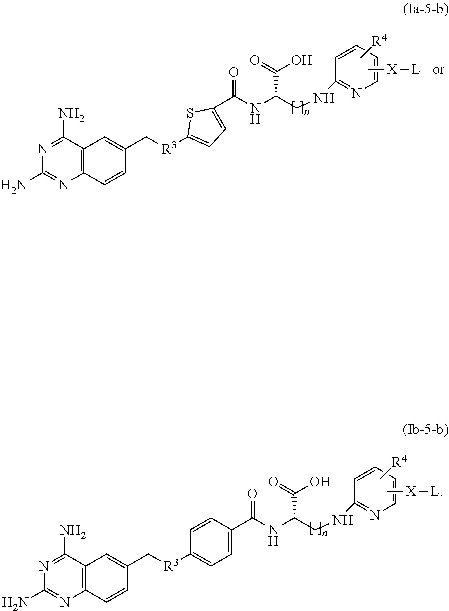

In yet another more specific embodiment, the invention provides a linker-drug compound of formula (Ia-5) or (Ib-5), wherein R¹ is NH₂, R² is CH, and R^{2′} is C-halogen of formula

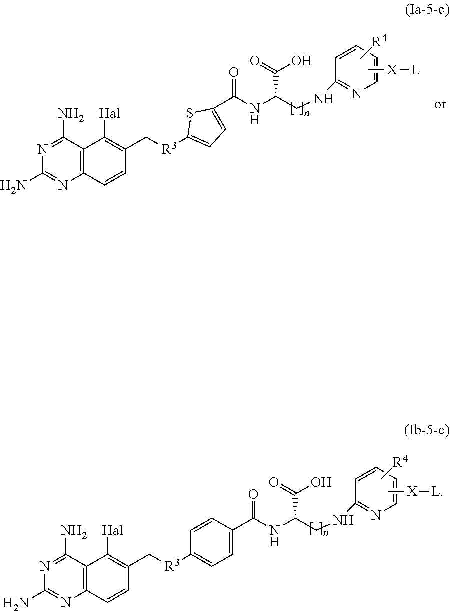

Even more specifically, the invention provides a linker-drug compound of formula (Ia-5) or (Ib-5), wherein R¹ is NH₂, R² is CH, and R^{2′} is C—Cl of formula

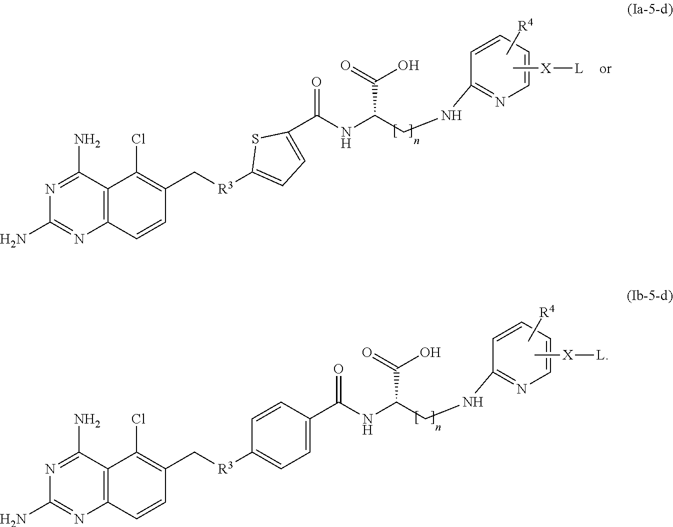

In a specific embodiment, R³ is NH, N(CH₃), or CH₂.

In another specific embodiment, R⁴ is —COOH and n is 4.

In another specific embodiment, R⁴ is H and n is 3.

Preferred linker-drug compounds according to the invention are

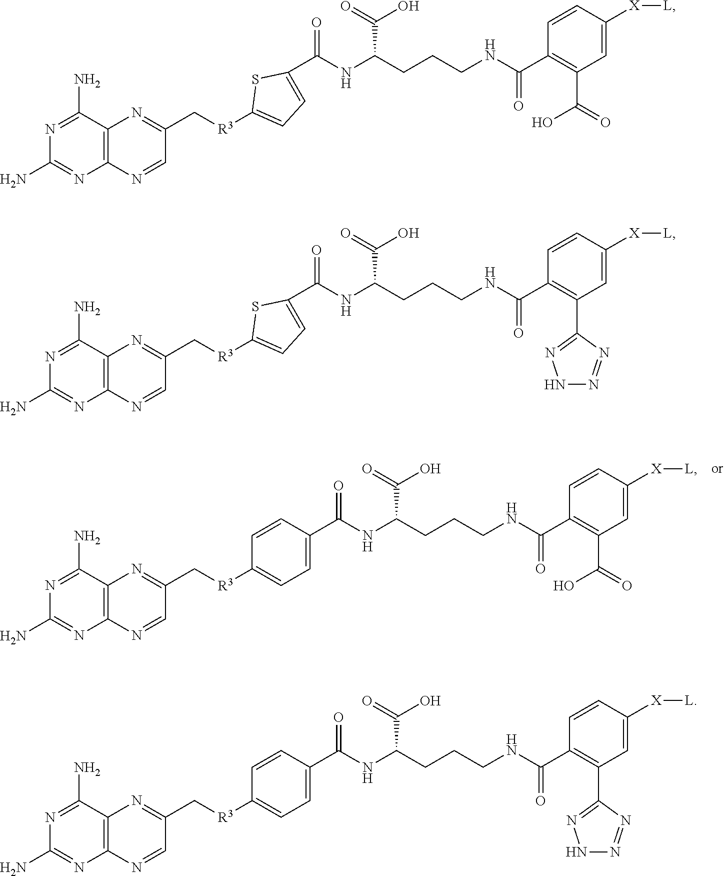

Other preferred linker-drug compounds according to the invention are

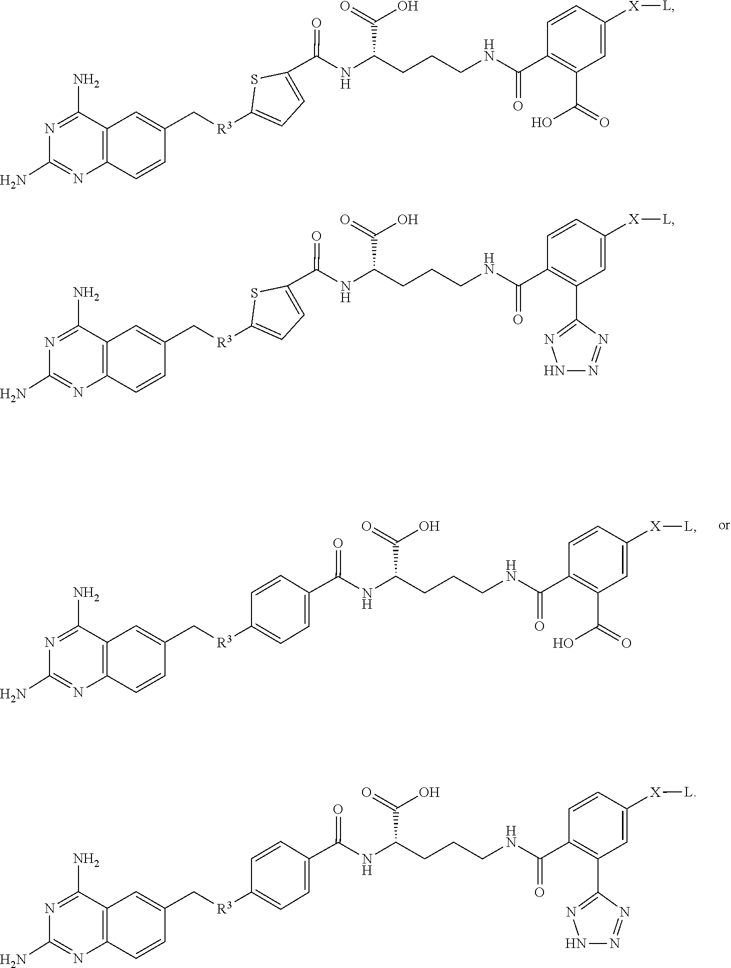

Other preferred linker-drug compounds according to the invention are

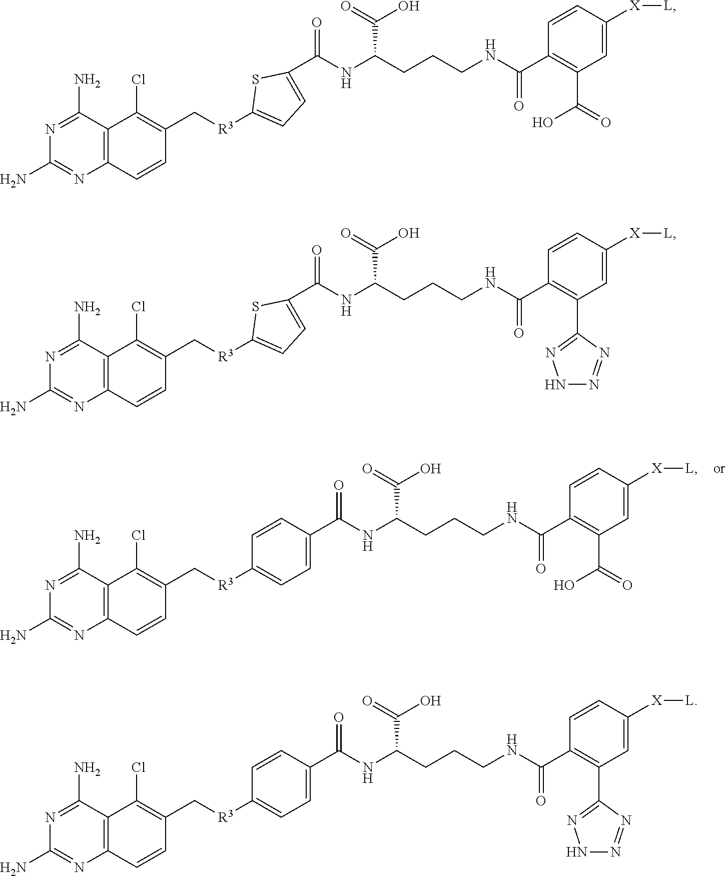

More preferred linker-drug compounds according to the invention are

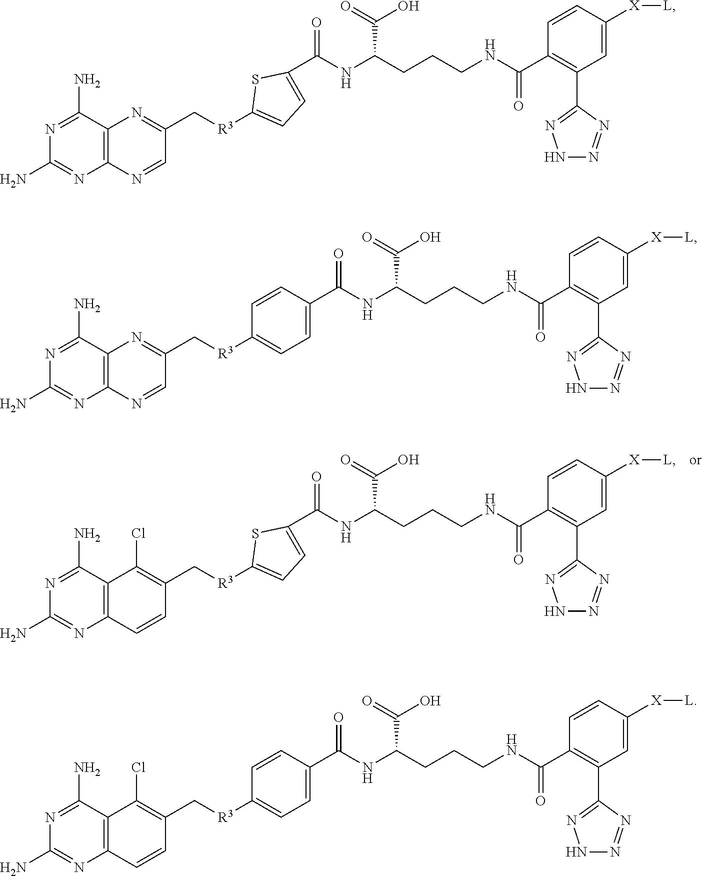

Even more preferred linker-drug compounds according to the invention are

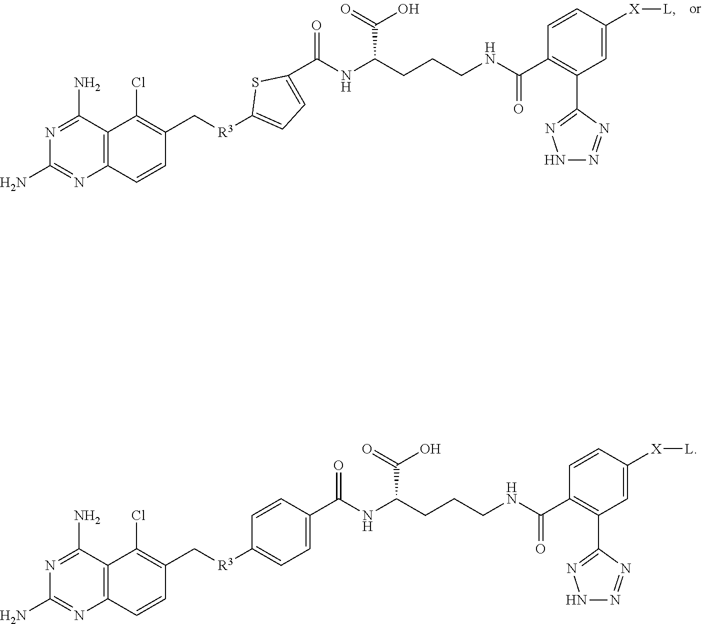

In a specific embodiment, R³ in any of the above preferred compounds is NH, N(CH₃), or CH₂.

The linker-drug compound according to the invention comprises a linker moiety. A linker is preferably a synthetic linker. The structure of a linker is such that the linker can be easily chemically attached to a small molecule drug, and so that the resulting linker-drug compound can be easily conjugated to a further substance such as for example a polypeptide to form an inhibitor conjugate. The choice of linker can influence the stability of such eventual conjugates when in circulation, and it can influence in what manner the small molecule drug compound is released, if it is released. Suitable linkers are for example described in Ducry et al, Bioconjugate Chem. 2010, 21, 5-13, King and Wagner, Bioconjugate Chem. 2014, 25, 825-839, Gordon et al, Bioconjugate Chem. 2015, 26, 2198-2215, Salomon et al, Mol. Pharmaceutics 2019, 16, 4817-4825, Chuprakov et al, Bioconjugate Chem. 2021, 32, 746-754, Giese et al, Bioconjugate Chem. 2021, 32, 2257-2267, Tsuchikama and An (DOI: 10.1007/s13238-016-0323-0), Polakis (DOI: 10.1124/pr.114.009373), Bargh et al. (DOI: 10.1039/c8cs00676h), WO 02/083180, WO 2004/043493, WO 2010/062171, WO 2011/133039, WO 2015/177360, and in WO 2018/069375. Linkers may be cleavable or non-cleavable, as described in e.g., Van Delft, F and Lambert, J. M., Chemical Linkers in Antibody-Drug Conjugates (ADCs), 1st Ed. Royal Society of Chemistry, ISBN-10: 1839162635 (2021). Cleavable linkers comprise moieties that can be cleaved, e.g., when exposed to lysosomal proteases or to an environment having an acidic pH or a higher reducing potential. Suitable cleavable linkers are known in the art and comprise e.g., a single amino acid, or a di-, tri- or tetrapeptide, i.e., a peptide composed of two, three or four amino acid residues. Additionally, the cleavable linker may comprise a selfimmolative moiety such as an ω-amino aminocarbonyl cyclization spacer, see Saari et al, J Med. Chem., 1990, 33, 97-101, a —NH—CH₂—O— moiety, or a sugar moiety as in Chuprakov et al, Bioconjugate Chem. 2021, 32, 746-754. Cleavage of the linker makes the antifolate moiety in the linker-drug compound according to the invention available to the surrounding environment. Non-cleavable linkers can still effectively release (a derivative of) the antifolate moiety from the linker-drug compound according to the invention, for example when a conjugated polypeptide is degraded in the lysosome. Non-cleavable linkers include e.g., succinimidyl-4-(N-maleimidomethyl(cyclohexane)-1-carboxylate and maleimidocaproic acid and analogs thereof.

Preferably, L is a cleavable linker.

In a preferred embodiment of the present invention, L is directly bonded to X via a carbonyl moiety.

To be able to conjugate a linker or linker-drug moiety to a polypeptide, such as an antibody, an antigen-binding fragment thereof or another targeting molecule, the side of the linker that will be (covalently) bonded to the antibody, antigen-binding fragment thereof or other targeting molecule typically contains a functional group that can react with an amino acid residue of the antibody, antigen-binding fragment thereof or other targeting molecule under relatively mild conditions. This functional group is referred to herein as a reactive moiety (RM). Examples of reactive moieties include, but are not limited to, carbamoyl halide, acyl halide, active ester, anhydride, α-halo acetyl, α-halo acetamide, maleimide, isocyanate, isothiocyanate, disulfide, thiol, hydrazine, hydrazide, sulfonyl chloride, aldehyde, methyl ketone, vinyl sulfone, halo methyl, methyl sulfonate, and cyclooctyn. Such amino acid residue with which the functional group reacts may be a natural or non-natural amino acid residue. The term “non-natural amino acid” as used herein is intended to represent a (synthetically) modified amino acid or the D stereoisomer of a naturally occurring amino acid.

Preferably, the amino acid residue with which the functional group reacts is a natural amino acid.

In a preferred embodiment of the present invention, RM is

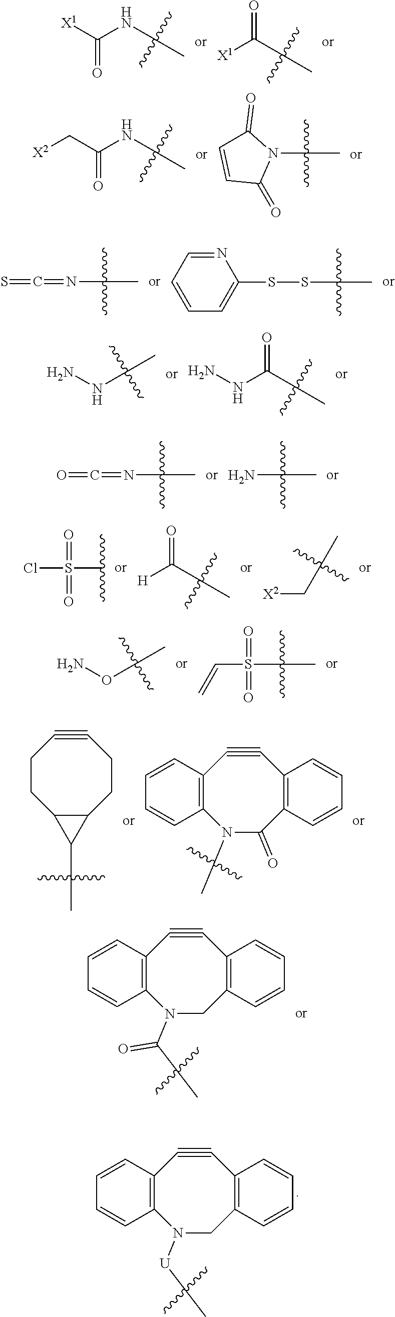

wherein
X¹ is selected from —Cl, —Br, —I, —F, —OH, —O—N-succinimide, —O-(4-nitrophenyl), —O-pentafluorophenyl, —O-tetrafluorophenyl, —O—C(O)—R⁹, and —O—C(O)—OR⁹, or C(O)—X¹ is an active ester;
X² is selected from —Cl, —Br, —I, —O-mesyl, —O-triflyl, and —O-tosyl;
R⁹ is selected from optionally substituted branched or unbranched C₁₋₁₀ alkyl, C₁₋₁₀ heteroalkyl, C₃₋₁₀ cycloalkyl, C₁₋₁₀ heterocycloalkyl, C₅₋₁₀ aryl or C₁₋₁₀ heteroaryl;
U is O or NR¹⁰; and
R¹⁰ is selected from H, a branched or unbranched C₁-C₁₂ alkyl or a C₄-C₁₂ (hetero)aryl group.

Preferably, RM is

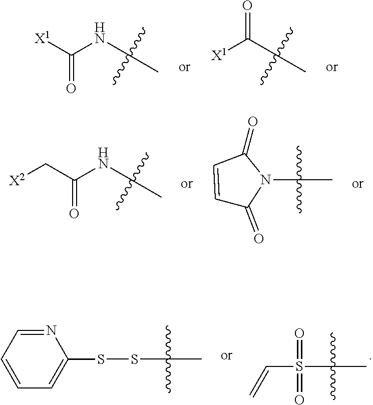

More preferably, RM is

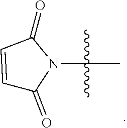 [SMILES: *N1C(=O)C=CC1=O]

The linker may further comprise one or more elongation spacers, such as

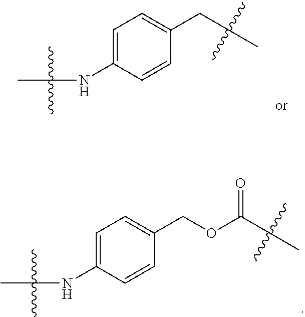 [SMILES: *Cc1ccc(N*)cc1 ; *Nc1ccc(COC(*)=O)cc1]

The linker may further comprise one or more elimination spacers, such as described in Alouane et al, Angew. Chem. Int. Ed. 2015, 54, 7492-7509, Deng et al, Macromol. Rapid Commun. 2020, 41, e1900531 or Bargh et al, Chem. Soc. Rev. 2019, 48, 4361-4374.

In one embodiment, the linker L is

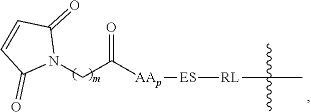

wherein
m is an integer ranging from 1 to 10, preferably 5;
AA is an amino acid, preferably a natural amino acid;
p is 0, 1, 2, 3, or 4;
ES is either absent or an elongation spacer selected from

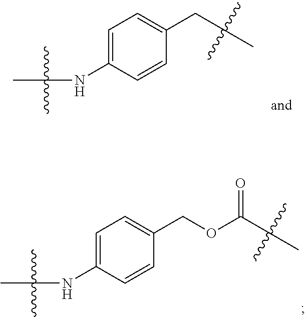 [SMILES: *Cc1ccc(N*)cc1 ; *Nc1ccc(COC(*)=O)cc1]

and
RL is either absent or an elimination spacer selected from

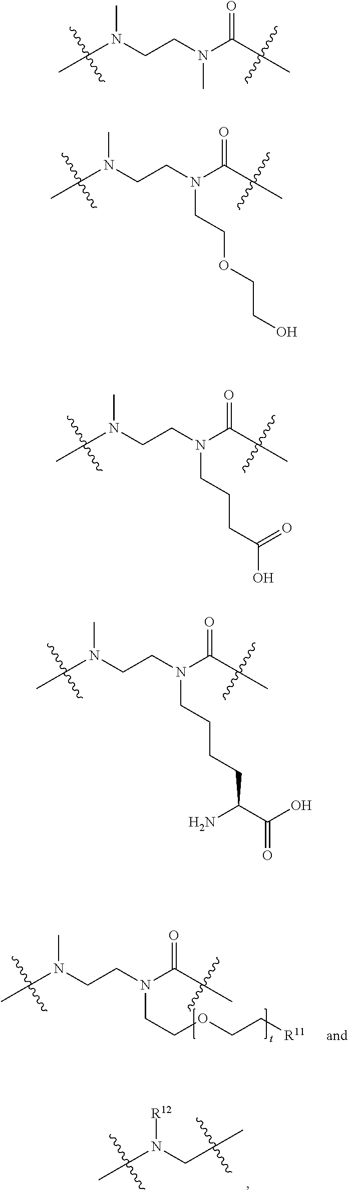

wherein t is an integer ranging from 1-10, R¹¹ is optionally substituted C₁₋₄ alkoxyl, and R¹² is H, optionally substituted C₁₋₆ alkyl, optionally substituted C₆₋₁₄ aryl, or optionally substituted C-linked C₃₋₈ heteroaryl.

In a preferred embodiment, m is 5, p is 0, ES and RL are absent, and L is

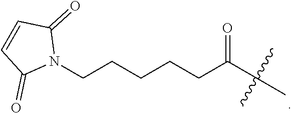 [SMILES: *C(=O)CCCCCN1C(=O)C=CC1=O]

In another preferred embodiment, AA is an amino acid selected from the group consisting of alanine, glycine, lysine, phenylalanine, valine, and citrulline.

In a first specific embodiment, p is 2 and AA₂ is phenylalanyllysine, valylalanine, valylcitrulline or valyllysine. More preferably, AA₂ is valylalanine or valylcitrulline. Most preferably, AA₂ is valylalanine or valylcitrulline and m is 5.

In a second specific embodiment, p is 3 and AA₃ is alanylphenylalanyllysine.

In a third specific embodiment, p is 4 and AA₄ is glycylglycylphenylalanylglycine.

In a preferred embodiment, m is 5, p is 2 and AA₂ is valylalanine or valylcitrulline.

Preferably, m is 5, p is 2, AA₂ is valylalanine, ES and RL are absent, and L is

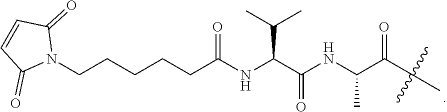 [SMILES: *C(=O)[C@H](C)NC(=O)[C@@H](NC(=O)CCCCCN1C(=O)C=CC1=O)C(C)C]

In another preferred embodiment, m is 5, p is 4, AA₄ is glycylglycylphenylalanylglycine, ES and RL are absent, and L is

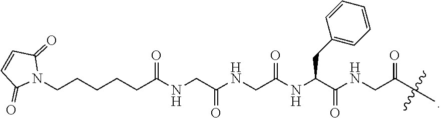 [SMILES: *C(=O)CNC(=O)[C@H](Cc1ccccc1)NC(=O)CNC(=O)CNC(=O)CCCCCN1C(=O)C=CC1=O]

In one embodiment, the linker L is

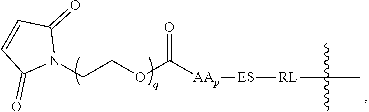

wherein
q is an integer ranging from 1 to 12, preferably 2;
AA is an amino acid, preferably a natural amino acid;
p is 0, 1, 2, 3, or 4;
ES is either absent or an elongation spacer selected from

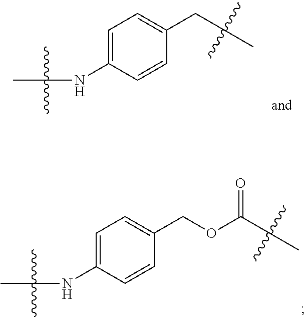 [SMILES: *Cc1ccc(N*)cc1 ; *Nc1ccc(COC(*)=O)cc1]

and
RL is either absent or an elimination spacer selected from

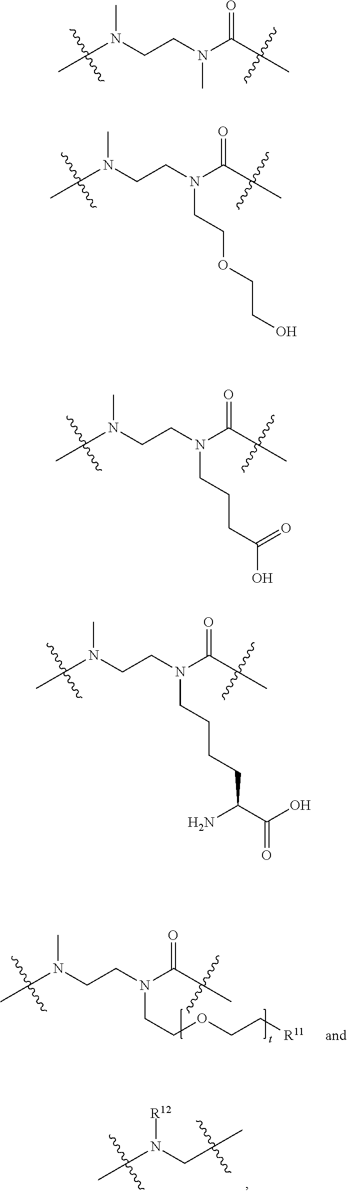

wherein t is an integer ranging from 1-10, R¹¹ is optionally substituted C₁₋₄ alkoxyl, and R¹² is H, optionally substituted C₁₋₆ alkyl, optionally substituted C₆₋₁₄ aryl or optionally substituted C-linked C₃₋₈ heteroaryl.

In a preferred embodiment, AA is an amino acid selected from the group consisting of alanine, glycine, lysine, phenylalanine, valine, and citrulline.

In a first specific embodiment, p is 2 and AA₂ is phenylalanyllysine, valylalanine, valylcitrulline or valyllysine. More preferably, AA₂ is valylalanine or valylcitrulline. Most preferably, AA₂ is valylalanine or valylcitrulline and q is 2.

In a second specific embodiment, p is 3 and AA₃ is alanylphenylalanyllysine.

In a third specific embodiment, p is 4 and AA₄ is glycylglycylphenylalanylglycine.

In a preferred embodiment, q is 2, p is 2 and AA₂ is valylalanine or valylcitrulline.

Preferably, q is 2, p is 2, AA₂ is valylcitrulline, RL is absent, ES is

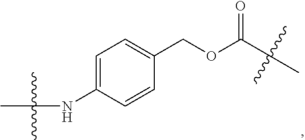 [SMILES: *Nc1ccc(COC(*)=O)cc1]

and L is

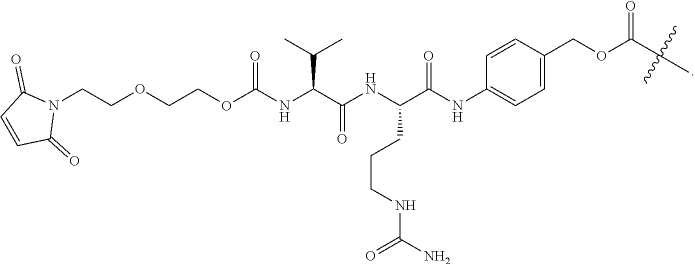 [SMILES: *C(=O)OCc1ccc(NC(=O)[C@H](CCCNC(N)=O)NC(=O)[C@@H](NC(=O)OCCOCCN2C(=O)C=CC2=O)C(C)C)cc1]

In another preferred embodiment, q is 2, p is 2, AA₂ is valylcitrulline, ES is

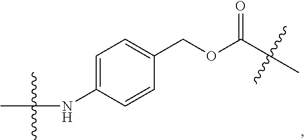 [SMILES: *Nc1ccc(COC(*)=O)cc1]

RL is

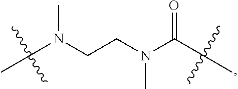 [SMILES: *C(=O)N(C)CCN(*)C]

and L is

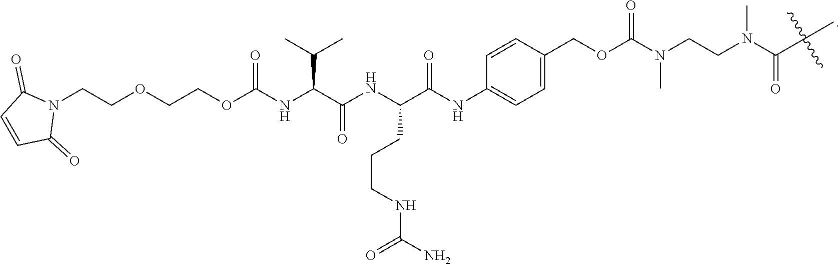 [SMILES: *C(=O)N(C)CCN(C)C(=O)OCc1ccc(NC(=O)[C@H](CCCNC(N)=O)NC(=O)[C@@H](NC(=O)OCCOCCN2C(=O)C=CC2=O)C(C)C)cc1]

In one embodiment, the linker L is

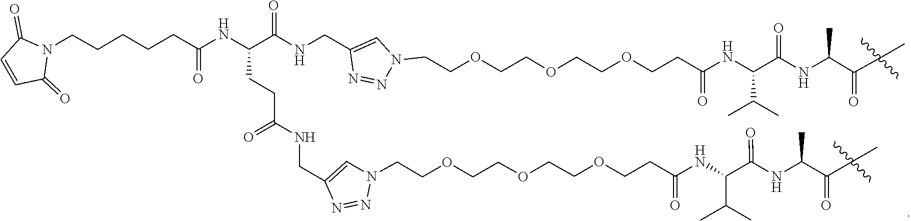 [SMILES: *C(=O)[C@H](C)NC(=O)[C@@H](NC(=O)CCOCCOCCOCCn1cc(CNC(=O)CC[C@H](NC(=O)CCCCCN2C(=O)C=CC2=O)C(=O)NCc2cn(CCOCCOCCOCCC(=O)N[C@H](C(=O)N[C@@H](C)C(*)=O)C(C)C)nn2)nn1)C(C)C]

The following are preferred linker-drug compounds according to the invention:

| | Small molecule drug moiety | |
|---|---|---|
| L | X | Remainder of the drug moiety |
| 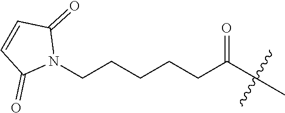 [SMILES: *C(=O)CCCCCN1C(=O)C=CC1=O] | —NH— | 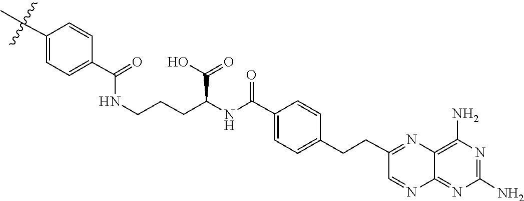 [SMILES: *c1ccc(C(=O)NCCC[C@H](NC(=O)c2ccc(CCc3cnc4nc(N)nc(N)c4n3)cc2)C(=O)O)cc1] |
| 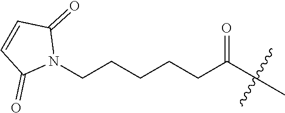 [SMILES: *C(=O)CCCCCN1C(=O)C=CC1=O] | —NH— | 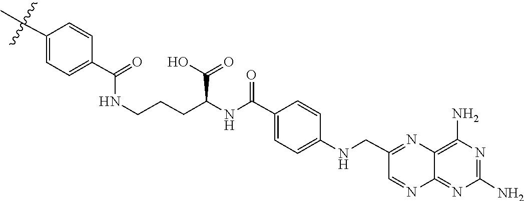 [SMILES: *c1ccc(C(=O)NCCC[C@H](NC(=O)c2ccc(NCc3cnc4nc(N)nc(N)c4n3)cc2)C(=O)O)cc1] |
| 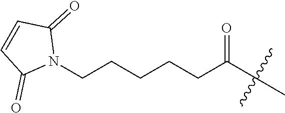 [SMILES: *C(=O)CCCCCN1C(=O)C=CC1=O] | —NH— | 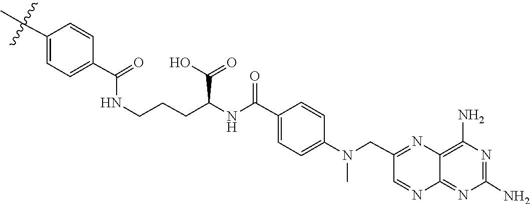 [SMILES: *c1ccc(C(=O)NCCC[C@H](NC(=O)c2ccc(N(C)Cc3cnc4nc(N)nc(N)c4n3)cc2)C(=O)O)cc1] |
| 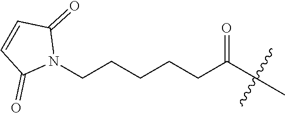 [SMILES: *C(=O)CCCCCN1C(=O)C=CC1=O] | —NH— | 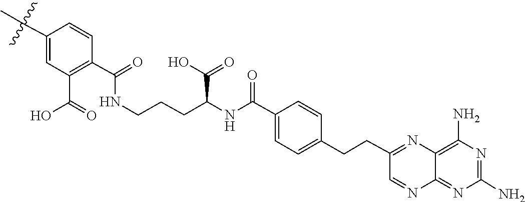 [SMILES: *c1ccc(C(=O)NCCC[C@H](NC(=O)c2ccc(CCc3cnc4nc(N)nc(N)c4n3)cc2)C(=O)O)c(C(=O)O)c1] |
| 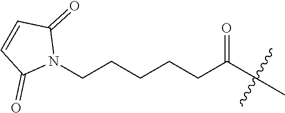 [SMILES: *C(=O)CCCCCN1C(=O)C=CC1=O] | —NH— | 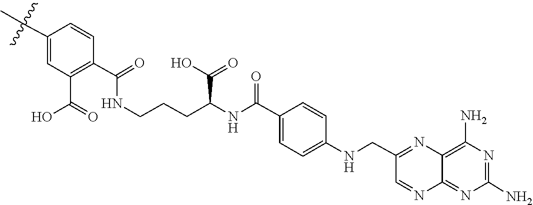 [SMILES: *c1ccc(C(=O)NCCC[C@H](NC(=O)c2ccc(NCc3cnc4nc(N)nc(N)c4n3)cc2)C(=O)O)c(C(=O)O)c1] |
| 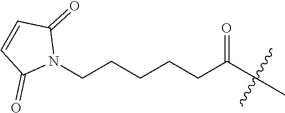 [SMILES: *C(=O)CCCCCN1C(=O)C=CC1=O] | —NH— | 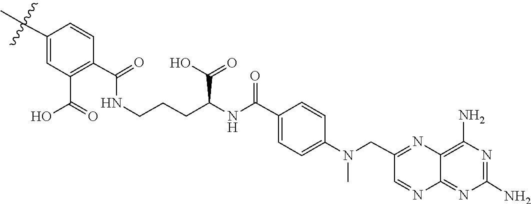 [SMILES: *c1ccc(C(=O)NCCC[C@H](NC(=O)c2ccc(N(C)Cc3cnc4nc(N)nc(N)c4n3)cc2)C(=O)O)c(C(=O)O)c1] |
| 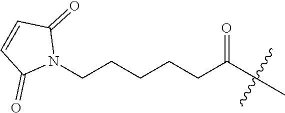 [SMILES: *C(=O)CCCCCN1C(=O)C=CC1=O] | —NH— | 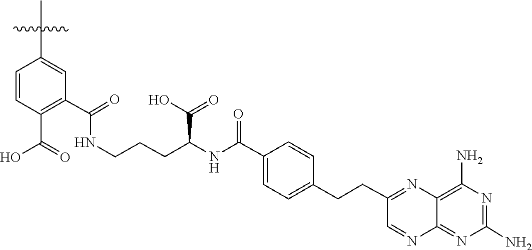 [SMILES: *c1ccc(C(=O)O)c(C(=O)NCCC[C@H](NC(=O)c2ccc(CCc3cnc4nc(N)nc(N)c4n3)cc2)C(=O)O)c1] |
| 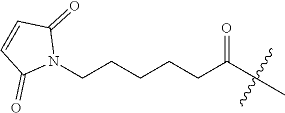 [SMILES: *C(=O)CCCCCN1C(=O)C=CC1=O] | —NH— | 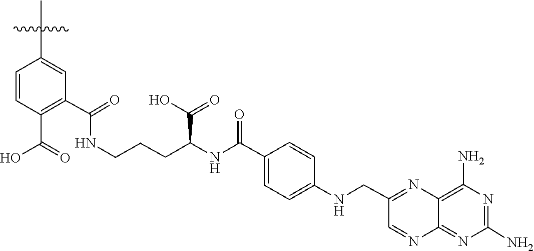 [SMILES: *c1ccc(C(=O)O)c(C(=O)NCCC[C@H](NC(=O)c2ccc(NCc3cnc4nc(N)nc(N)c4n3)cc2)C(=O)O)c1] |
| 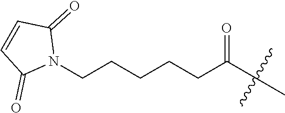 [SMILES: *C(=O)CCCCCN1C(=O)C=CC1=O] | —NH— | 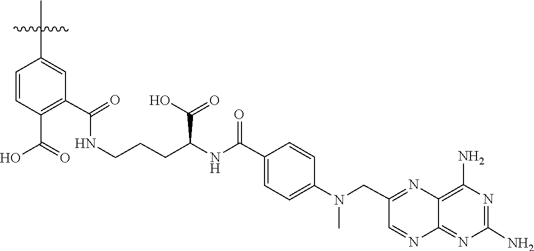 [SMILES: *c1ccc(C(=O)O)c(C(=O)NCCC[C@H](NC(=O)c2ccc(N(C)Cc3cnc4nc(N)nc(N)c4n3)cc2)C(=O)O)c1] |
| 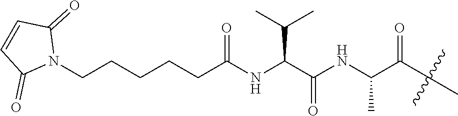 [SMILES: *C(=O)[C@H](C)NC(=O)[C@@H](NC(=O)CCCCCN1C(=O)C=CC1=O)C(C)C] | —NH— | 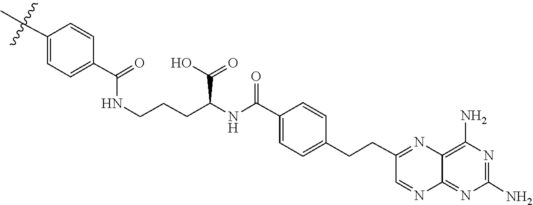 [SMILES: *c1ccc(C(=O)NCCC[C@H](NC(=O)c2ccc(CCc3cnc4nc(N)nc(N)c4n3)cc2)C(=O)O)cc1] |
| 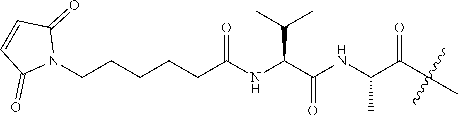 [SMILES: *C(=O)[C@H](C)NC(=O)[C@@H](NC(=O)CCCCCN1C(=O)C=CC1=O)C(C)C] | —NH— | 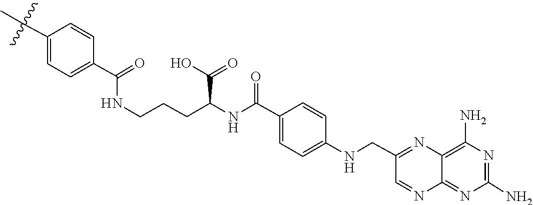 [SMILES: *c1ccc(C(=O)NCCC[C@H](NC(=O)c2ccc(NCc3cnc4nc(N)nc(N)c4n3)cc2)C(=O)O)cc1] |
| 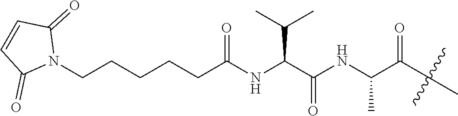 [SMILES: *C(=O)[C@H](C)NC(=O)[C@@H](NC(=O)CCCCCN1C(=O)C=CC1=O)C(C)C] | —NH— | 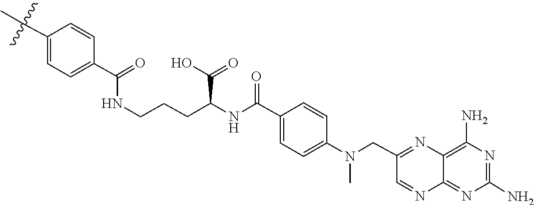 [SMILES: *c1ccc(C(=O)NCCC[C@H](NC(=O)c2ccc(N(C)Cc3cnc4nc(N)nc(N)c4n3)cc2)C(=O)O)cc1] |
| 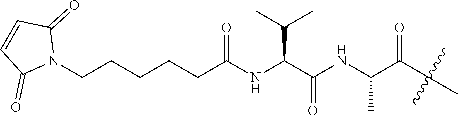 [SMILES: *C(=O)[C@H](C)NC(=O)[C@@H](NC(=O)CCCCCN1C(=O)C=CC1=O)C(C)C] | —NH— | 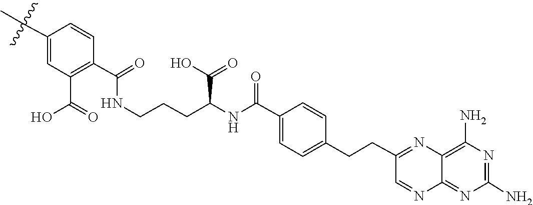 [SMILES: *c1ccc(C(=O)NCCC[C@H](NC(=O)c2ccc(CCc3cnc4nc(N)nc(N)c4n3)cc2)C(=O)O)c(C(=O)O)c1] |
| 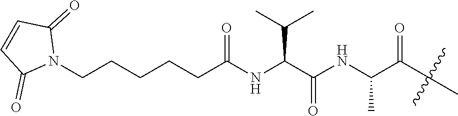 [SMILES: *C(=O)[C@H](C)NC(=O)[C@@H](NC(=O)CCCCCN1C(=O)C=CC1=O)C(C)C] | —NH— | 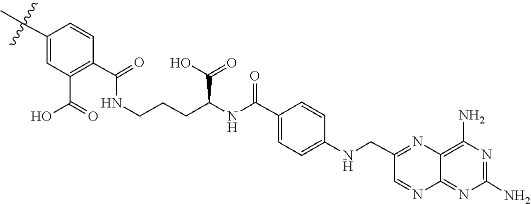 [SMILES: *c1ccc(C(=O)NCCC[C@H](NC(=O)c2ccc(NCc3cnc4nc(N)nc(N)c4n3)cc2)C(=O)O)c(C(=O)O)c1] |
| 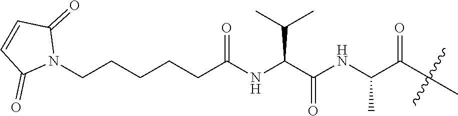 [SMILES: *C(=O)[C@H](C)NC(=O)[C@@H](NC(=O)CCCCCN1C(=O)C=CC1=O)C(C)C] | —NH— | 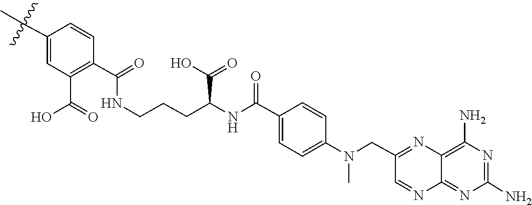 [SMILES: *c1ccc(C(=O)NCCC[C@H](NC(=O)c2ccc(N(C)Cc3cnc4nc(N)nc(N)c4n3)cc2)C(=O)O)c(C(=O)O)c1] |
| 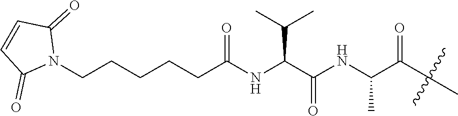 [SMILES: *C(=O)[C@H](C)NC(=O)[C@@H](NC(=O)CCCCCN1C(=O)C=CC1=O)C(C)C] | —NH— | 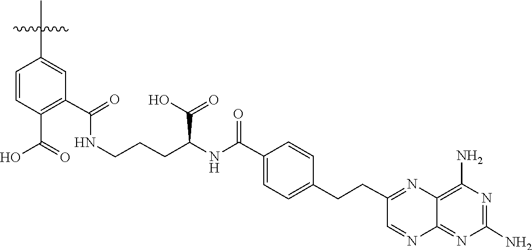 [SMILES: *c1ccc(C(=O)O)c(C(=O)NCCC[C@H](NC(=O)c2ccc(CCc3cnc4nc(N)nc(N)c4n3)cc2)C(=O)O)c1] |
| 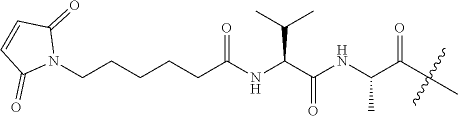 [SMILES: *C(=O)[C@H](C)NC(=O)[C@@H](NC(=O)CCCCCN1C(=O)C=CC1=O)C(C)C] | —NH— | 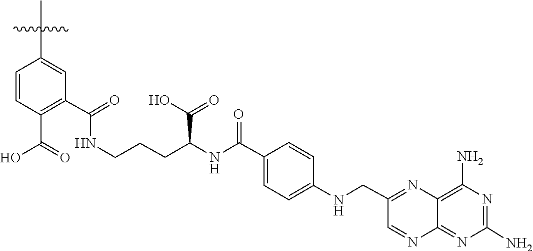 [SMILES: *c1ccc(C(=O)O)c(C(=O)NCCC[C@H](NC(=O)c2ccc(NCc3cnc4nc(N)nc(N)c4n3)cc2)C(=O)O)c1] |
| 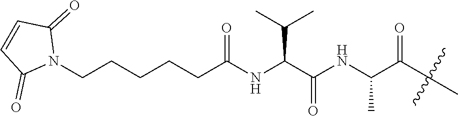 [SMILES: *C(=O)[C@H](C)NC(=O)[C@@H](NC(=O)CCCCCN1C(=O)C=CC1=O)C(C)C] | —NH— | 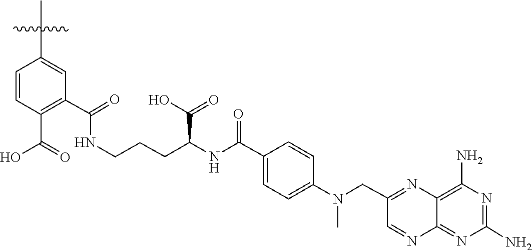 [SMILES: *c1ccc(C(=O)O)c(C(=O)NCCC[C@H](NC(=O)c2ccc(N(C)Cc3cnc4nc(N)nc(N)c4n3)cc2)C(=O)O)c1] |
| 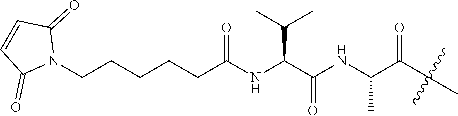 [SMILES: *C(=O)[C@H](C)NC(=O)[C@@H](NC(=O)CCCCCN1C(=O)C=CC1=O)C(C)C] | —NH— | 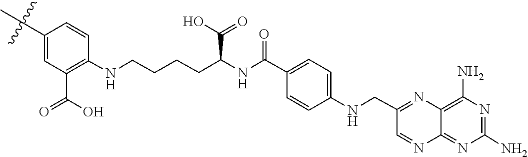 [SMILES: *c1ccc(NCCCC[C@H](NC(=O)c2ccc(NCc3cnc4nc(N)nc(N)c4n3)cc2)C(=O)O)c(C(=O)O)c1] |
| 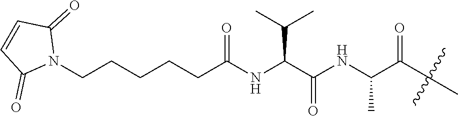 [SMILES: *C(=O)[C@H](C)NC(=O)[C@@H](NC(=O)CCCCCN1C(=O)C=CC1=O)C(C)C] | —NH— | 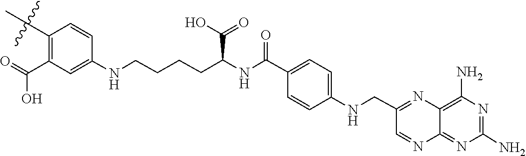 [SMILES: *c1ccc(NCCCC[C@H](NC(=O)c2ccc(NCc3cnc4nc(N)nc(N)c4n3)cc2)C(=O)O)cc1C(=O)O] |
| 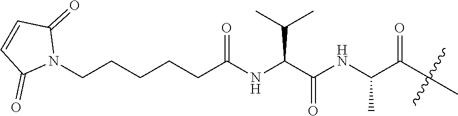 [SMILES: *C(=O)[C@H](C)NC(=O)[C@@H](NC(=O)CCCCCN1C(=O)C=CC1=O)C(C)C] | —NH— | 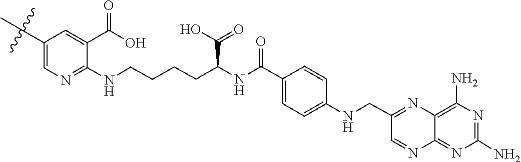 [SMILES: *c1cnc(NCCCC[C@H](NC(=O)c2ccc(NCc3cnc4nc(N)nc(N)c4n3)cc2)C(=O)O)c(C(=O)O)c1] |
| 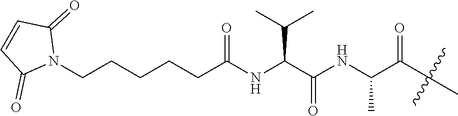 [SMILES: *C(=O)[C@H](C)NC(=O)[C@@H](NC(=O)CCCCCN1C(=O)C=CC1=O)C(C)C] | —NH— | 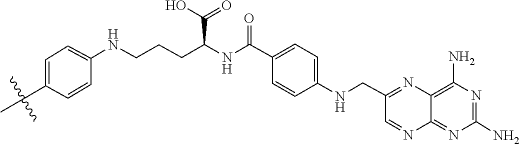 [SMILES: *c1ccc(NCCC[C@H](NC(=O)c2ccc(NCc3cnc4nc(N)nc(N)c4n3)cc2)C(=O)O)cc1] |
| 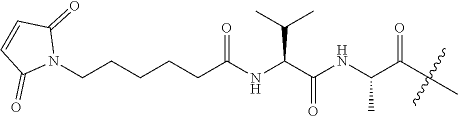 [SMILES: *C(=O)[C@H](C)NC(=O)[C@@H](NC(=O)CCCCCN1C(=O)C=CC1=O)C(C)C] | —NH— | 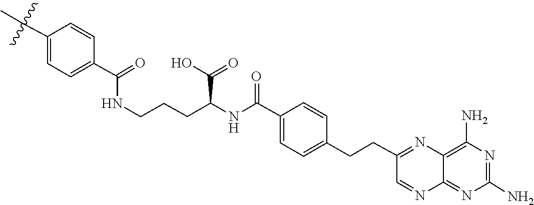 [SMILES: *c1ccc(C(=O)NCCC[C@H](NC(=O)c2ccc(CCc3cnc4nc(N)nc(N)c4n3)cc2)C(=O)O)cc1] |
| 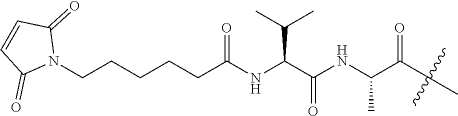 [SMILES: *C(=O)[C@H](C)NC(=O)[C@@H](NC(=O)CCCCCN1C(=O)C=CC1=O)C(C)C] | —NH— | 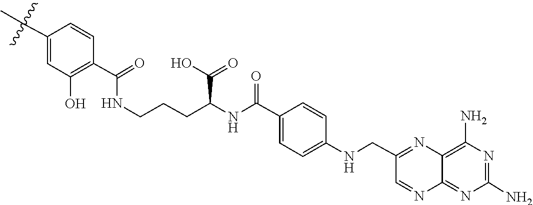 [SMILES: *c1ccc(C(=O)NCCC[C@H](NC(=O)c2ccc(NCc3cnc4nc(N)nc(N)c4n3)cc2)C(=O)O)c(O)c1] |
| 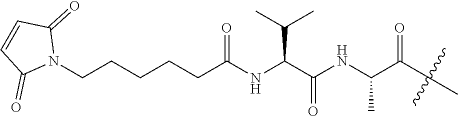 [SMILES: *C(=O)[C@H](C)NC(=O)[C@@H](NC(=O)CCCCCN1C(=O)C=CC1=O)C(C)C] | —NH— | 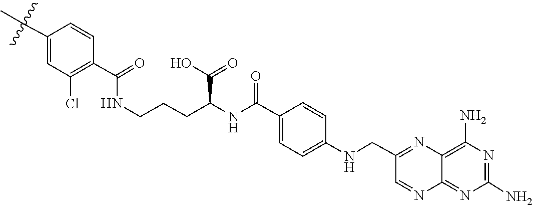 [SMILES: *c1ccc(C(=O)NCCC[C@H](NC(=O)c2ccc(NCc3cnc4nc(N)nc(N)c4n3)cc2)C(=O)O)c(Cl)c1] |
| 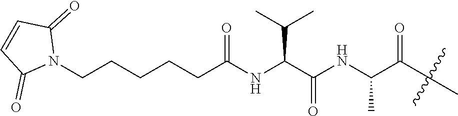 [SMILES: *C(=O)[C@H](C)NC(=O)[C@@H](NC(=O)CCCCCN1C(=O)C=CC1=O)C(C)C] | —NH— | 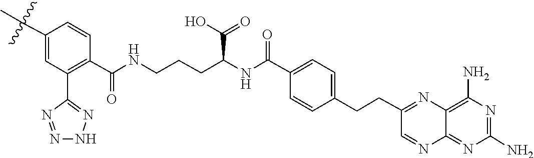 [SMILES: *c1ccc(C(=O)NCCC[C@H](NC(=O)c2ccc(CCc3cnc4nc(N)nc(N)c4n3)cc2)C(=O)O)c(-c2nn[nH]n2)c1] |
| 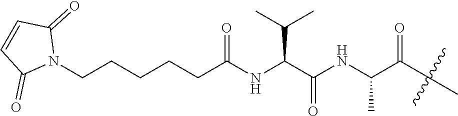 [SMILES: *C(=O)[C@H](C)NC(=O)[C@@H](NC(=O)CCCCCN1C(=O)C=CC1=O)C(C)C] | —NH— | 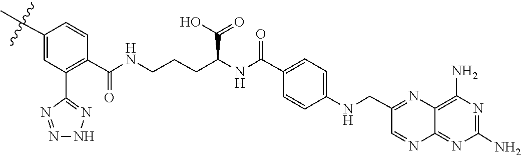 [SMILES: *c1ccc(C(=O)NCCC[C@H](NC(=O)c2ccc(NCc3cnc4nc(N)nc(N)c4n3)cc2)C(=O)O)c(-c2nn[nH]n2)c1] |
| 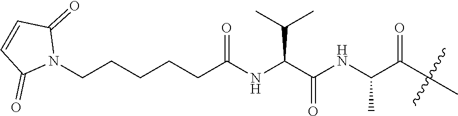 [SMILES: *C(=O)[C@H](C)NC(=O)[C@@H](NC(=O)CCCCCN1C(=O)C=CC1=O)C(C)C] | —NH— | 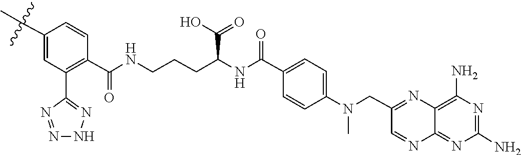 [SMILES: *c1ccc(C(=O)NCCC[C@H](NC(=O)c2ccc(N(C)Cc3cnc4nc(N)nc(N)c4n3)cc2)C(=O)O)c(-c2nn[nH]n2)c1] |
| 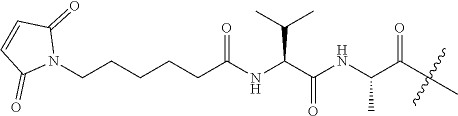 [SMILES: *C(=O)[C@H](C)NC(=O)[C@@H](NC(=O)CCCCCN1C(=O)C=CC1=O)C(C)C] | —NH— | 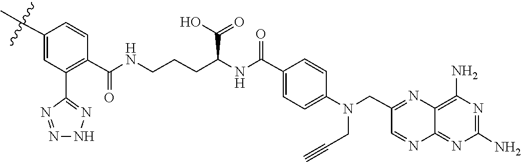 [SMILES: *c1ccc(C(=O)NCCC[C@H](NC(=O)c2ccc(N(CC#C)Cc3cnc4nc(N)nc(N)c4n3)cc2)C(=O)O)c(-c2nn[nH]n2)c1] |
| 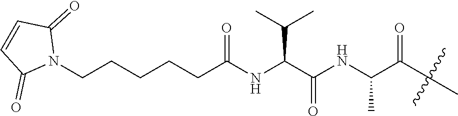 [SMILES: *C(=O)[C@H](C)NC(=O)[C@@H](NC(=O)CCCCCN1C(=O)C=CC1=O)C(C)C] | —NH— | 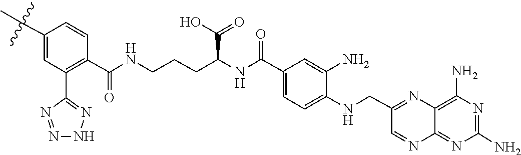 [SMILES: *c1ccc(C(=O)NCCC[C@H](NC(=O)c2ccc(NCc3cnc4nc(N)nc(N)c4n3)c(N)c2)C(=O)O)c(-c2nn[nH]n2)c1] |
| 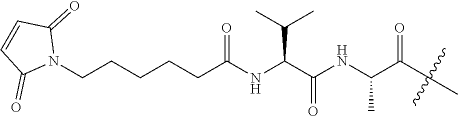 [SMILES: *C(=O)[C@H](C)NC(=O)[C@@H](NC(=O)CCCCCN1C(=O)C=CC1=O)C(C)C] | —NH— | 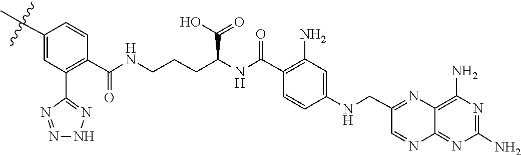 [SMILES: *c1ccc(C(=O)NCCC[C@H](NC(=O)c2ccc(NCc3cnc4nc(N)nc(N)c4n3)cc2N)C(=O)O)c(-c2nn[nH]n2)c1] |
| 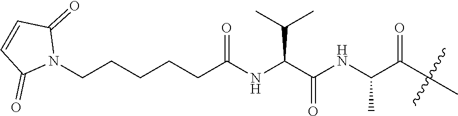 [SMILES: *C(=O)[C@H](C)NC(=O)[C@@H](NC(=O)CCCCCN1C(=O)C=CC1=O)C(C)C] | —NH— | 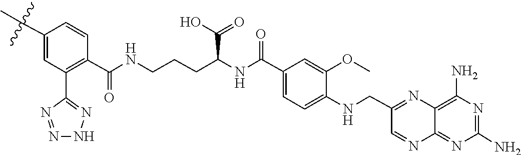 [SMILES: *c1ccc(C(=O)NCCC[C@H](NC(=O)c2ccc(NCc3cnc4nc(N)nc(N)c4n3)c(OC)c2)C(=O)O)c(-c2nn[nH]n2)c1] |
| 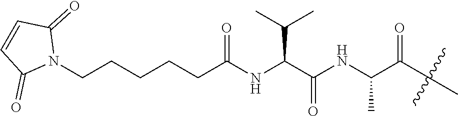 [SMILES: *C(=O)[C@H](C)NC(=O)[C@@H](NC(=O)CCCCCN1C(=O)C=CC1=O)C(C)C] | —NH— | 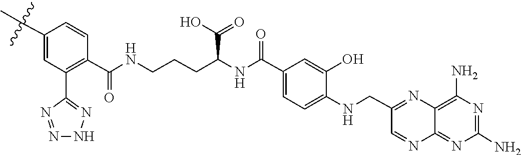 [SMILES: *c1ccc(C(=O)NCCC[C@H](NC(=O)c2ccc(NCc3cnc4nc(N)nc(N)c4n3)c(O)c2)C(=O)O)c(-c2nn[nH]n2)c1] |
| 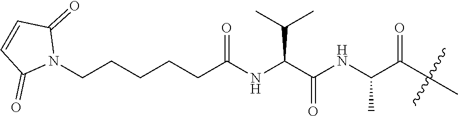 [SMILES: *C(=O)[C@H](C)NC(=O)[C@@H](NC(=O)CCCCCN1C(=O)C=CC1=O)C(C)C] | —NH— | 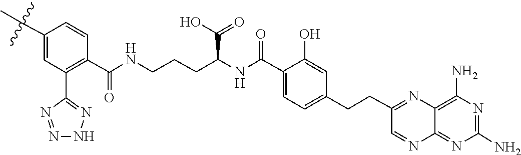 [SMILES: *c1ccc(C(=O)NCCC[C@H](NC(=O)c2ccc(CCc3cnc4nc(N)nc(N)c4n3)cc2O)C(=O)O)c(-c2nn[nH]n2)c1] |
| 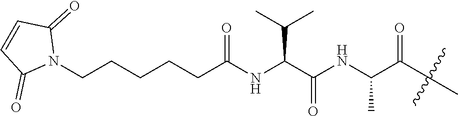 [SMILES: *C(=O)[C@H](C)NC(=O)[C@@H](NC(=O)CCCCCN1C(=O)C=CC1=O)C(C)C] | —NH— | 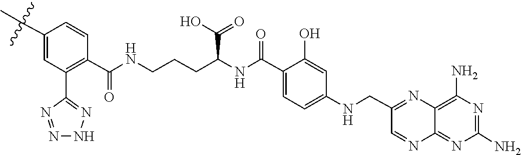 [SMILES: *c1ccc(C(=O)NCCC[C@H](NC(=O)c2ccc(NCc3cnc4nc(N)nc(N)c4n3)cc2O)C(=O)O)c(-c2nn[nH]n2)c1] |
| 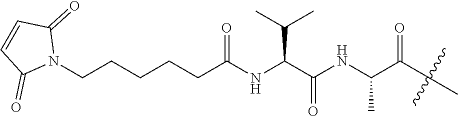 [SMILES: *C(=O)[C@H](C)NC(=O)[C@@H](NC(=O)CCCCCN1C(=O)C=CC1=O)C(C)C] | —NH— | 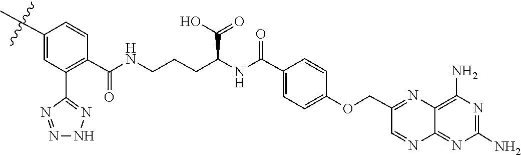 [SMILES: *c1ccc(C(=O)NCCC[C@H](NC(=O)c2ccc(OCc3cnc4nc(N)nc(N)c4n3)cc2)C(=O)O)c(-c2nn[nH]n2)c1] |
| 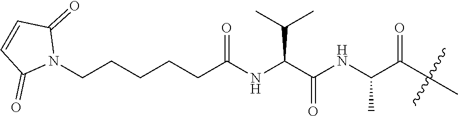 [SMILES: *C(=O)[C@H](C)NC(=O)[C@@H](NC(=O)CCCCCN1C(=O)C=CC1=O)C(C)C] | —NH— | 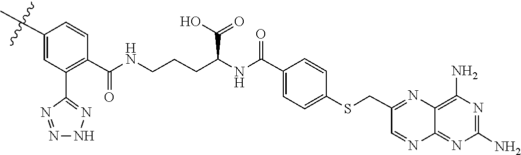 [SMILES: *c1ccc(C(=O)NCCC[C@H](NC(=O)c2ccc(SCc3cnc4nc(N)nc(N)c4n3)cc2)C(=O)O)c(-c2nn[nH]n2)c1] |
| 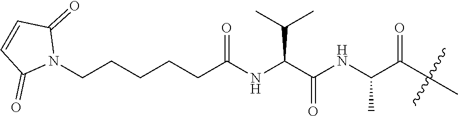 [SMILES: *C(=O)[C@H](C)NC(=O)[C@@H](NC(=O)CCCCCN1C(=O)C=CC1=O)C(C)C] | —NH— | 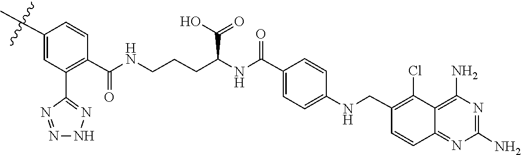 [SMILES: *c1ccc(C(=O)NCCC[C@H](NC(=O)c2ccc(NCc3ccc4nc(N)nc(N)c4c3Cl)cc2)C(=O)O)c(-c2nn[nH]n2)c1] |
| 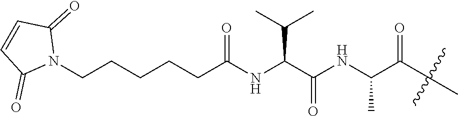 [SMILES: *C(=O)[C@H](C)NC(=O)[C@@H](NC(=O)CCCCCN1C(=O)C=CC1=O)C(C)C] | —NH— | 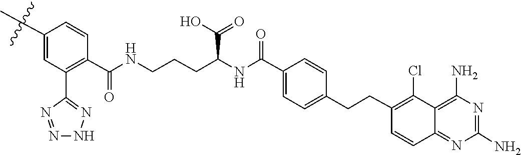 [SMILES: *c1ccc(C(=O)NCCC[C@H](NC(=O)c2ccc(CCc3ccc4nc(N)nc(N)c4c3Cl)cc2)C(=O)O)c(-c2nn[nH]n2)c1] |
| 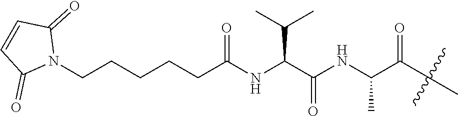 [SMILES: *C(=O)[C@H](C)NC(=O)[C@@H](NC(=O)CCCCCN1C(=O)C=CC1=O)C(C)C] | —NH— | 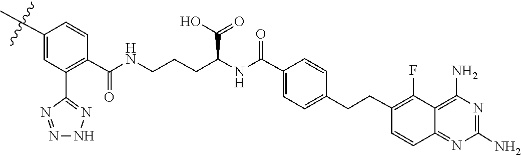 [SMILES: *c1ccc(C(=O)NCCC[C@H](NC(=O)c2ccc(CCc3ccc4nc(N)nc(N)c4c3F)cc2)C(=O)O)c(-c2nn[nH]n2)c1] |
| 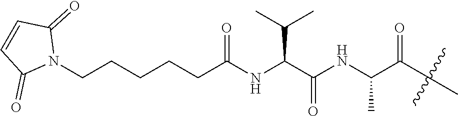 [SMILES: *C(=O)[C@H](C)NC(=O)[C@@H](NC(=O)CCCCCN1C(=O)C=CC1=O)C(C)C] | —NH— | 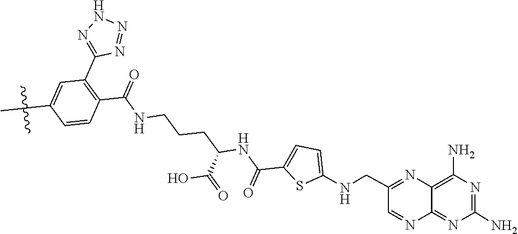 [SMILES: *c1ccc(C(=O)NCCC[C@H](NC(=O)c2ccc(NCc3cnc4nc(N)nc(N)c4n3)s2)C(=O)O)c(-c2nn[nH]n2)c1] |
| 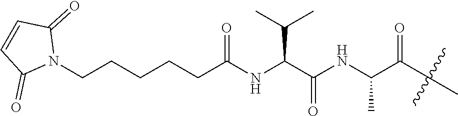 [SMILES: *C(=O)[C@H](C)NC(=O)[C@@H](NC(=O)CCCCCN1C(=O)C=CC1=O)C(C)C] | —NH— | 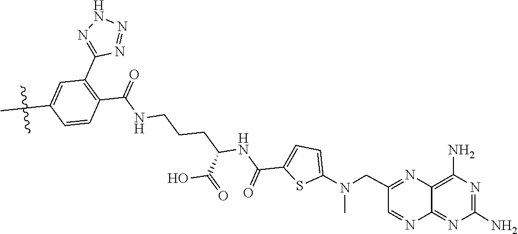 [SMILES: *c1ccc(C(=O)NCCC[C@H](NC(=O)c2ccc(N(C)Cc3cnc4nc(N)nc(N)c4n3)s2)C(=O)O)c(-c2nn[nH]n2)c1] |
| 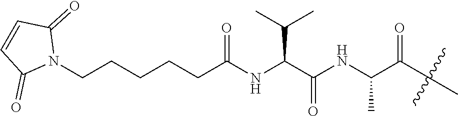 [SMILES: *C(=O)[C@H](C)NC(=O)[C@@H](NC(=O)CCCCCN1C(=O)C=CC1=O)C(C)C] | —NH— | 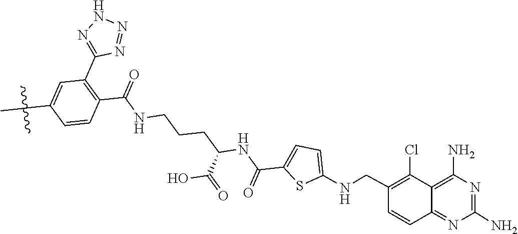 [SMILES: *c1ccc(C(=O)NCCC[C@H](NC(=O)c2ccc(NCc3ccc4nc(N)nc(N)c4c3Cl)s2)C(=O)O)c(-c2nn[nH]n2)c1] |
| 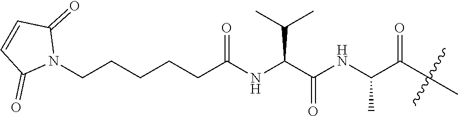 [SMILES: *C(=O)[C@H](C)NC(=O)[C@@H](NC(=O)CCCCCN1C(=O)C=CC1=O)C(C)C] | —NH— | 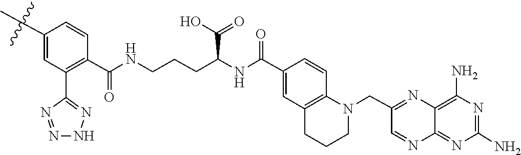 [SMILES: *c1ccc(C(=O)NCCC[C@H](NC(=O)c2ccc3c(c2)CCCN3Cc2cnc3nc(N)nc(N)c3n2)C(=O)O)c(-c2nn[nH]n2)c1] |
| 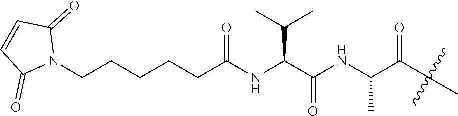 [SMILES: *C(=O)[C@H](C)NC(=O)[C@@H](NC(=O)CCCCCN1C(=O)C=CC1=O)C(C)C] | —NH— | 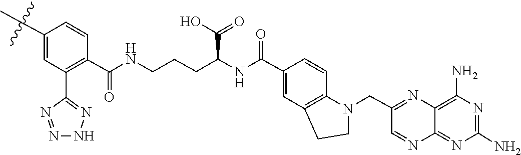 [SMILES: *c1ccc(C(=O)NCCC[C@H](NC(=O)c2ccc3c(c2)CCN3Cc2cnc3nc(N)nc(N)c3n2)C(=O)O)c(-c2nn[nH]n2)c1] |
| 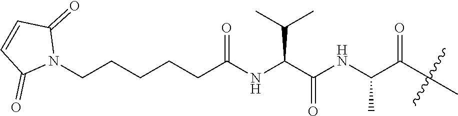 [SMILES: *C(=O)[C@H](C)NC(=O)[C@@H](NC(=O)CCCCCN1C(=O)C=CC1=O)C(C)C] | —NH— | 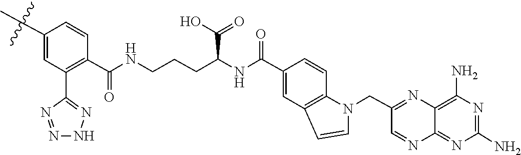 [SMILES: *c1ccc(C(=O)NCCC[C@H](NC(=O)c2ccc3c(ccn3Cc3cnc4nc(N)nc(N)c4n3)c2)C(=O)O)c(-c2nn[nH]n2)c1] |
| 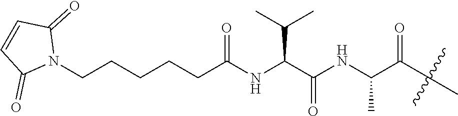 [SMILES: *C(=O)[C@H](C)NC(=O)[C@@H](NC(=O)CCCCCN1C(=O)C=CC1=O)C(C)C] | —NH— | 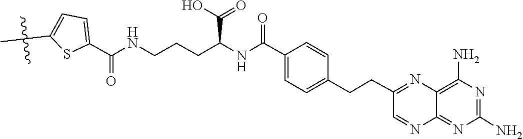 [SMILES: *c1ccc(C(=O)NCCC[C@H](NC(=O)c2ccc(CCc3cnc4nc(N)nc(N)c4n3)cc2)C(=O)O)s1] |
| 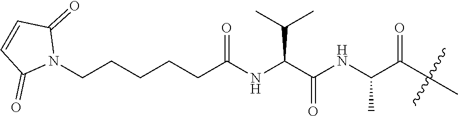 [SMILES: *C(=O)[C@H](C)NC(=O)[C@@H](NC(=O)CCCCCN1C(=O)C=CC1=O)C(C)C] | —NH— | 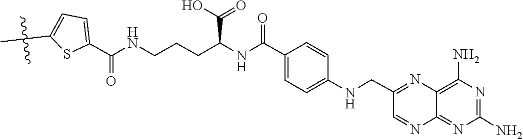 [SMILES: *c1ccc(C(=O)NCCC[C@H](NC(=O)c2ccc(NCc3cnc4nc(N)nc(N)c4n3)cc2)C(=O)O)s1] |
| 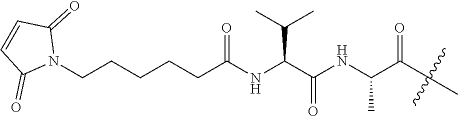 [SMILES: *C(=O)[C@H](C)NC(=O)[C@@H](NC(=O)CCCCCN1C(=O)C=CC1=O)C(C)C] | —NH— | 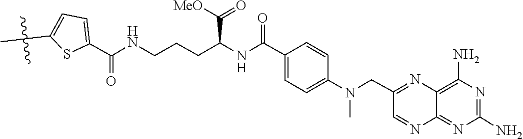 [SMILES: *c1ccc(C(=O)NCCC[C@H](NC(=O)c2ccc(N(C)Cc3cnc4nc(N)nc(N)c4n3)cc2)C(=O)OC)s1] |
| 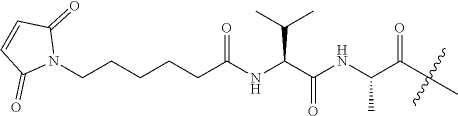 [SMILES: *C(=O)[C@H](C)NC(=O)[C@@H](NC(=O)CCCCCN1C(=O)C=CC1=O)C(C)C] | —NH— | 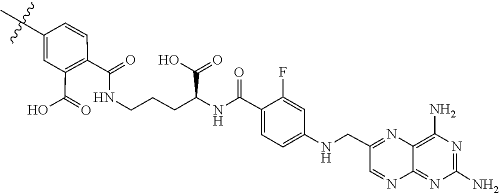 [SMILES: *c1ccc(C(=O)NCCC[C@H](NC(=O)c2ccc(NCc3cnc4nc(N)nc(N)c4n3)cc2F)C(=O)O)c(C(=O)O)c1] |
| 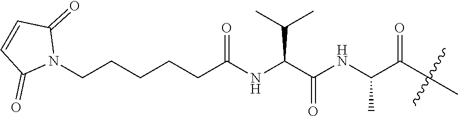 [SMILES: *C(=O)[C@H](C)NC(=O)[C@@H](NC(=O)CCCCCN1C(=O)C=CC1=O)C(C)C] | —NH— | 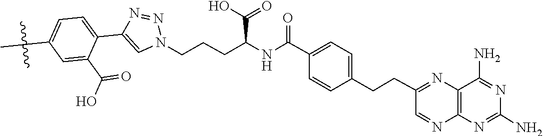 [SMILES: *c1ccc(-c2cn(CCC[C@H](NC(=O)c3ccc(CCc4cnc5nc(N)nc(N)c5n4)cc3)C(=O)O)nn2)c(C(=O)O)c1] |
| 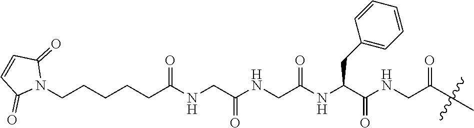 [SMILES: *C(=O)CNC(=O)[C@H](Cc1ccccc1)NC(=O)CNC(=O)CNC(=O)CCCCCN1C(=O)C=CC1=O] | —NH— | 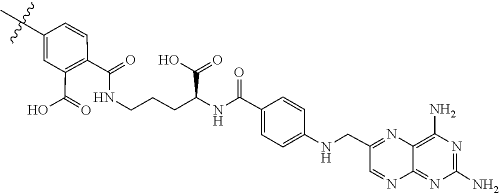 [SMILES: *c1ccc(C(=O)NCCC[C@H](NC(=O)c2ccc(NCc3cnc4nc(N)nc(N)c4n3)cc2)C(=O)O)c(C(=O)O)c1] |
| 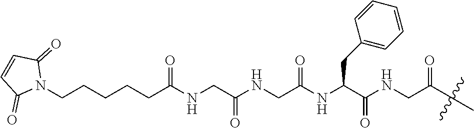 [SMILES: *C(=O)CNC(=O)[C@H](Cc1ccccc1)NC(=O)CNC(=O)CNC(=O)CCCCCN1C(=O)C=CC1=O] | —NH— | 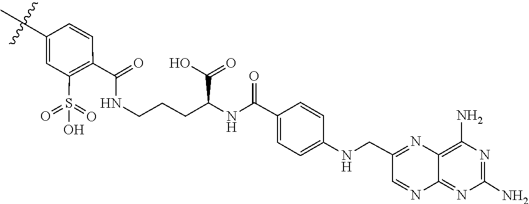 [SMILES: *c1ccc(C(=O)NCCC[C@H](NC(=O)c2ccc(NCc3cnc4nc(N)nc(N)c4n3)cc2)C(=O)O)c(S(=O)(=O)O)c1] |
| 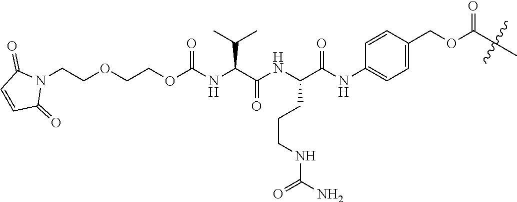 [SMILES: *C(=O)OCc1ccc(NC(=O)[C@H](CCCNC(N)=O)NC(=O)[C@@H](NC(=O)OCCOCCN2C(=O)C=CC2=O)C(C)C)cc1] | —NH— | 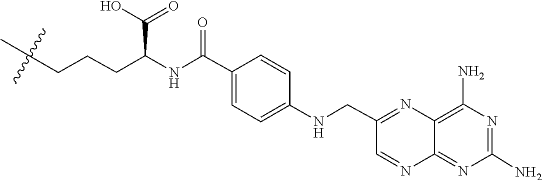 [SMILES: *CCC[C@H](NC(=O)c1ccc(NCc2cnc3nc(N)nc(N)c3n2)cc1)C(=O)O] |
| 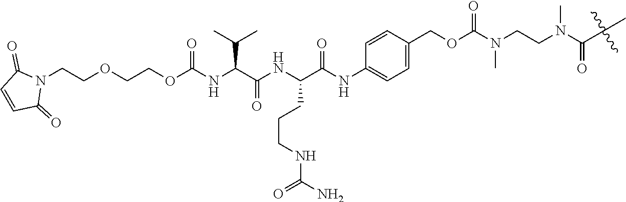 [SMILES: *C(=O)N(C)CCN(C)C(=O)OCc1ccc(NC(=O)[C@H](CCCNC(N)=O)NC(=O)[C@@H](NC(=O)OCCOCCN2C(=O)C=CC2=O)C(C)C)cc1] | —NH— | 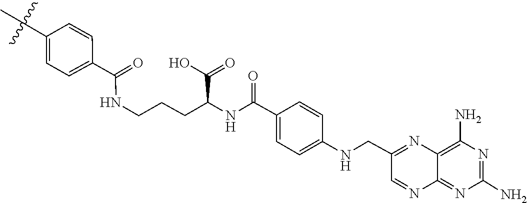 [SMILES: *c1ccc(C(=O)NCCC[C@H](NC(=O)c2ccc(NCc3cnc4nc(N)nc(N)c4n3)cc2)C(=O)O)cc1] |
| 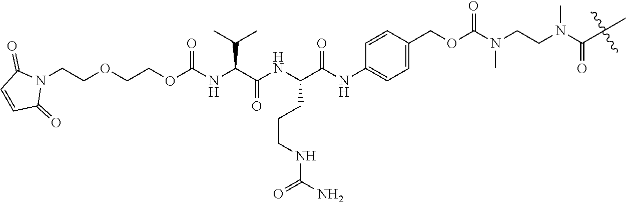 [SMILES: *C(=O)N(C)CCN(C)C(=O)OCc1ccc(NC(=O)[C@H](CCCNC(N)=O)NC(=O)[C@@H](NC(=O)OCCOCCN2C(=O)C=CC2=O)C(C)C)cc1] | —NH— | 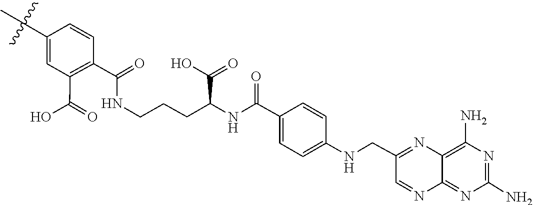 [SMILES: *c1ccc(C(=O)NCCC[C@H](NC(=O)c2ccc(NCc3cnc4nc(N)nc(N)c4n3)cc2)C(=O)O)c(C(=O)O)c1] |
| 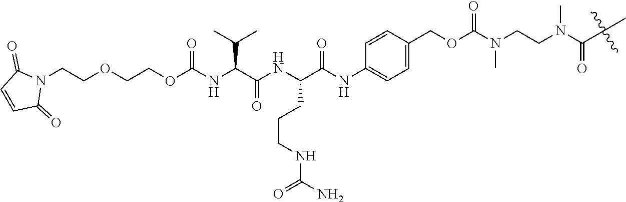 [SMILES: *C(=O)N(C)CCN(C)C(=O)OCc1ccc(NC(=O)[C@H](CCCNC(N)=O)NC(=O)[C@@H](NC(=O)OCCOCCN2C(=O)C=CC2=O)C(C)C)cc1] | —NH— | 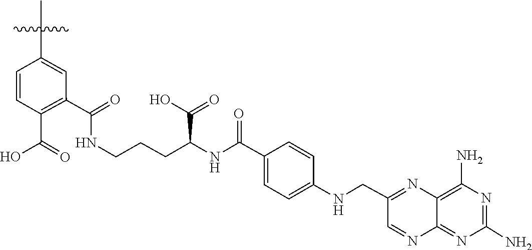 [SMILES: *c1ccc(C(=O)O)c(C(=O)NCCC[C@H](NC(=O)c2ccc(NCc3cnc4nc(N)nc(N)c4n3)cc2)C(=O)O)c1] |
| 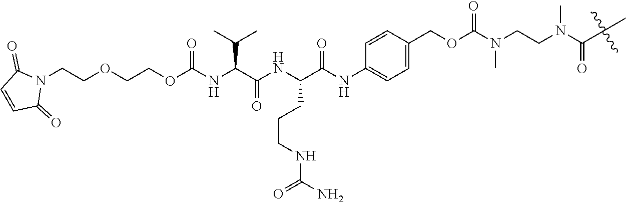 [SMILES: *C(=O)N(C)CCN(C)C(=O)OCc1ccc(NC(=O)[C@H](CCCNC(N)=O)NC(=O)[C@@H](NC(=O)OCCOCCN2C(=O)C=CC2=O)C(C)C)cc1] | —NH— | 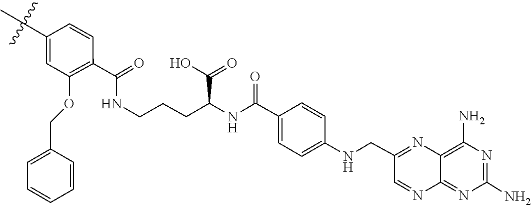 [SMILES: *c1ccc(C(=O)NCCC[C@H](NC(=O)c2ccc(NCc3cnc4nc(N)nc(N)c4n3)cc2)C(=O)O)c(OCc2ccccc2)c1] |
| 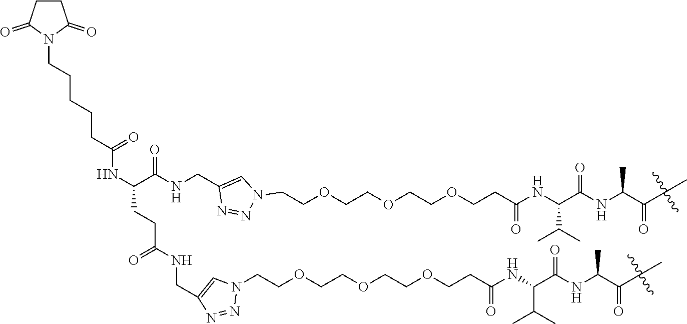 [SMILES: *C(=O)[C@H](C)NC(=O)[C@@H](NC(=O)CCOCCOCCOCCn1cc(CNC(=O)CC[C@H](NC(=O)CCCCCN2C(=O)CCC2=O)C(=O)NCc2cn(CCOCCOCCOCCC(=O)N[C@H](C(=O)N[C@@H](C)C(*)=O)C(C)C)nn2)nn1)C(C)C] | —NH— | 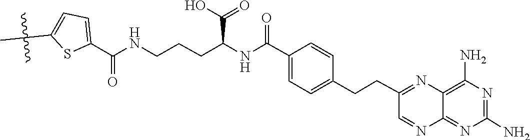 [SMILES: *c1ccc(C(=O)NCCC[C@H](NC(=O)c2ccc(CCc3cnc4nc(N)nc(N)c4n3)cc2)C(=O)O)s1] |

Two small molecule drug moieties are attached to L.

More preferred linker-drug compounds according to the invention are

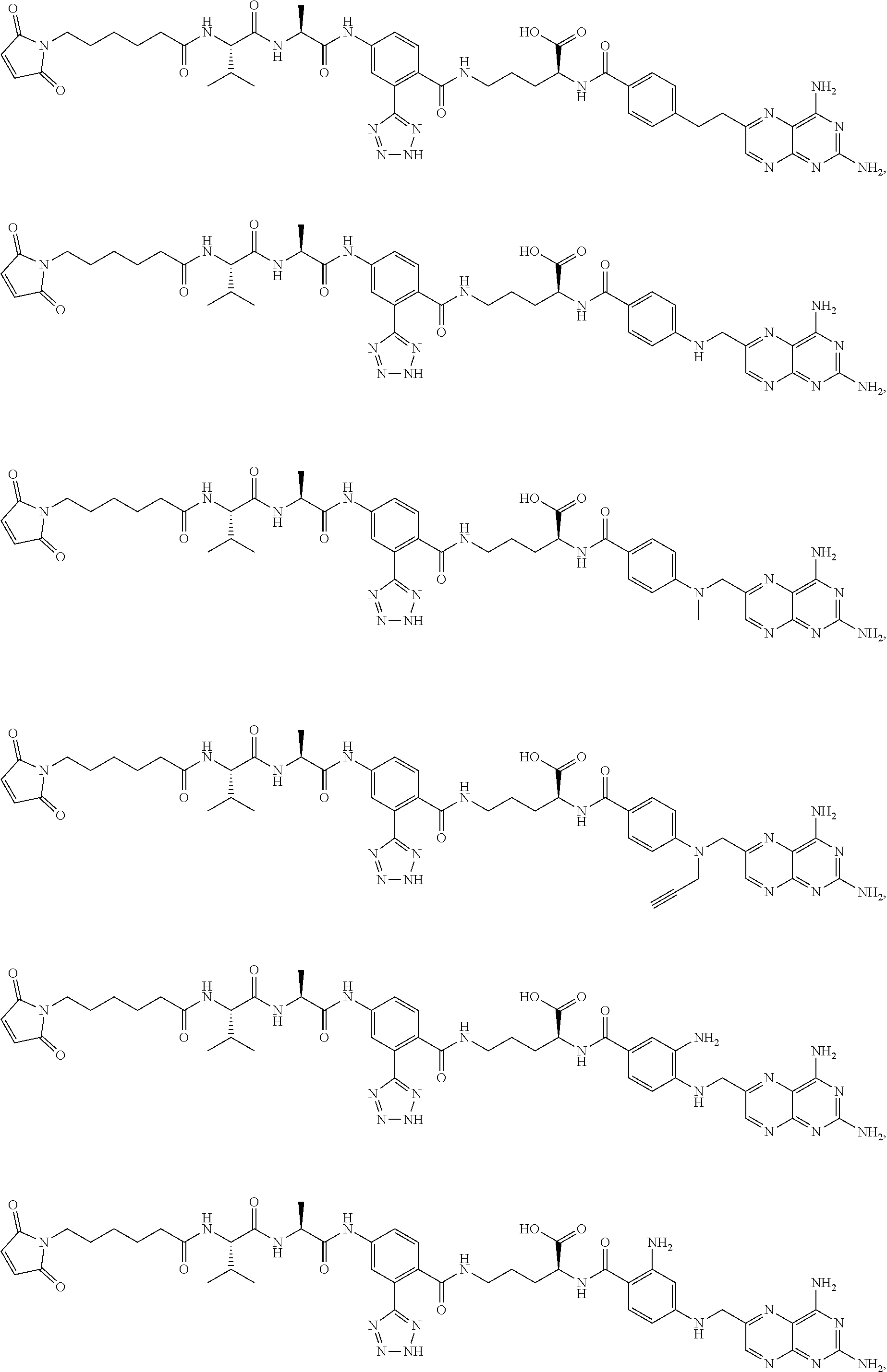

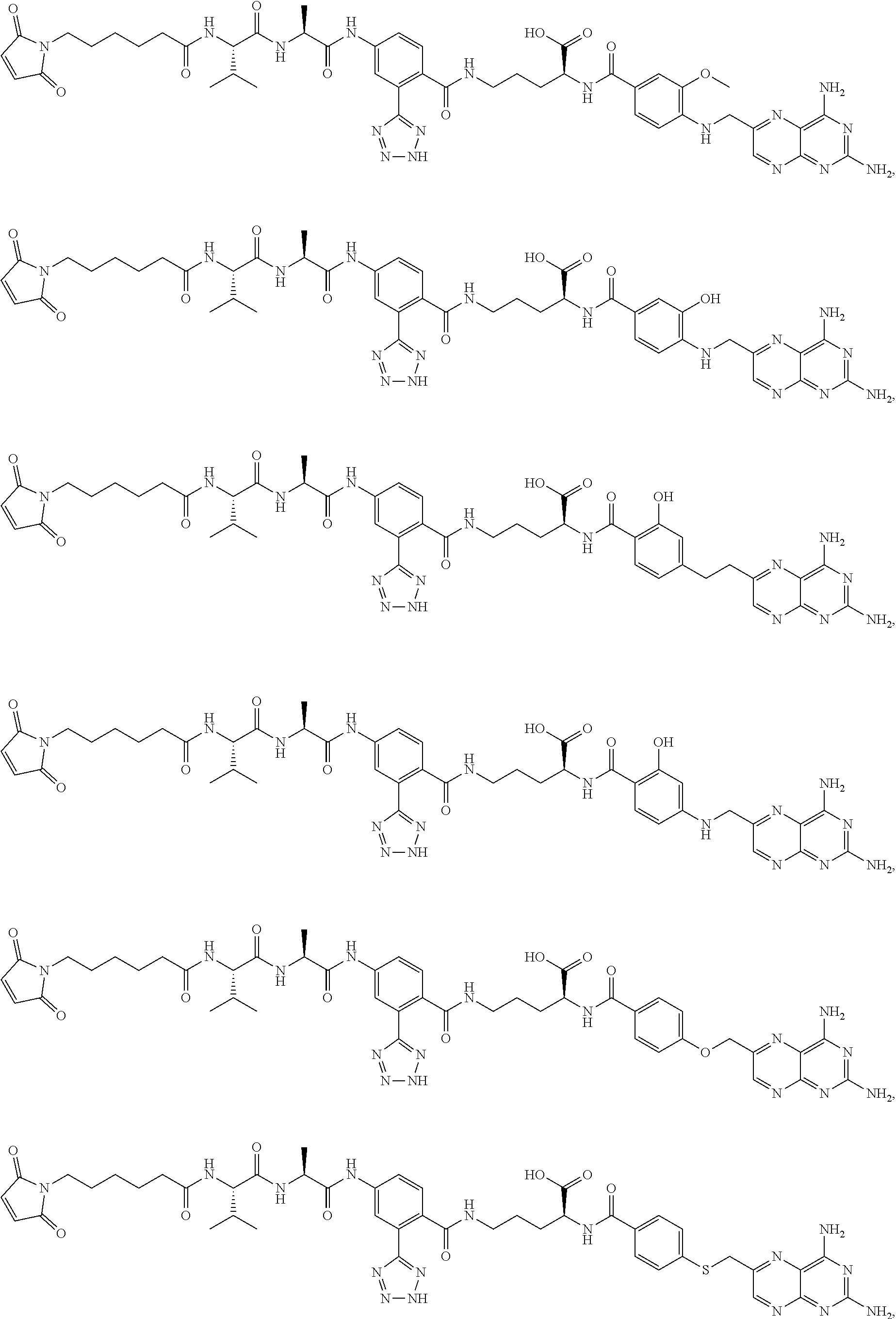

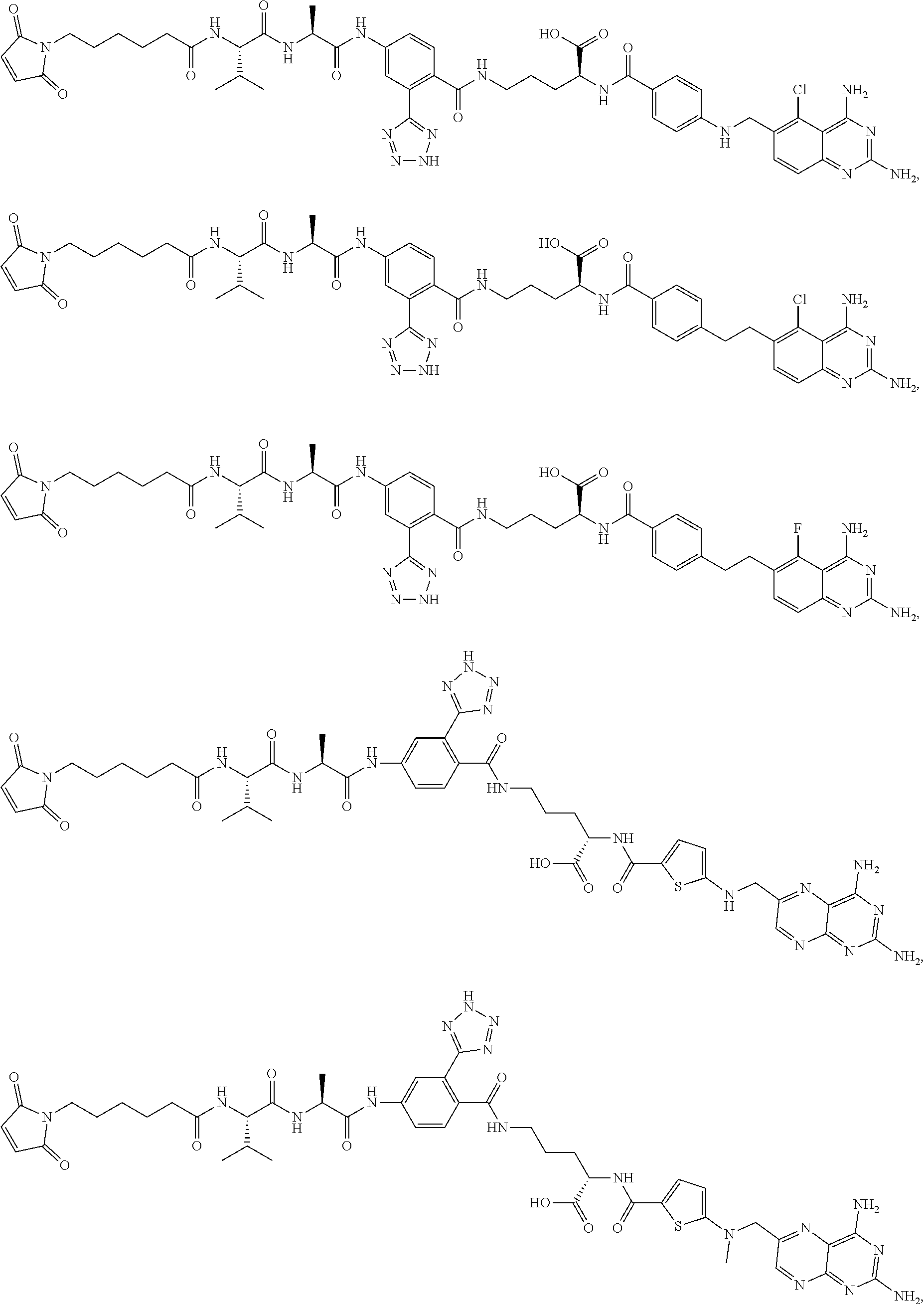

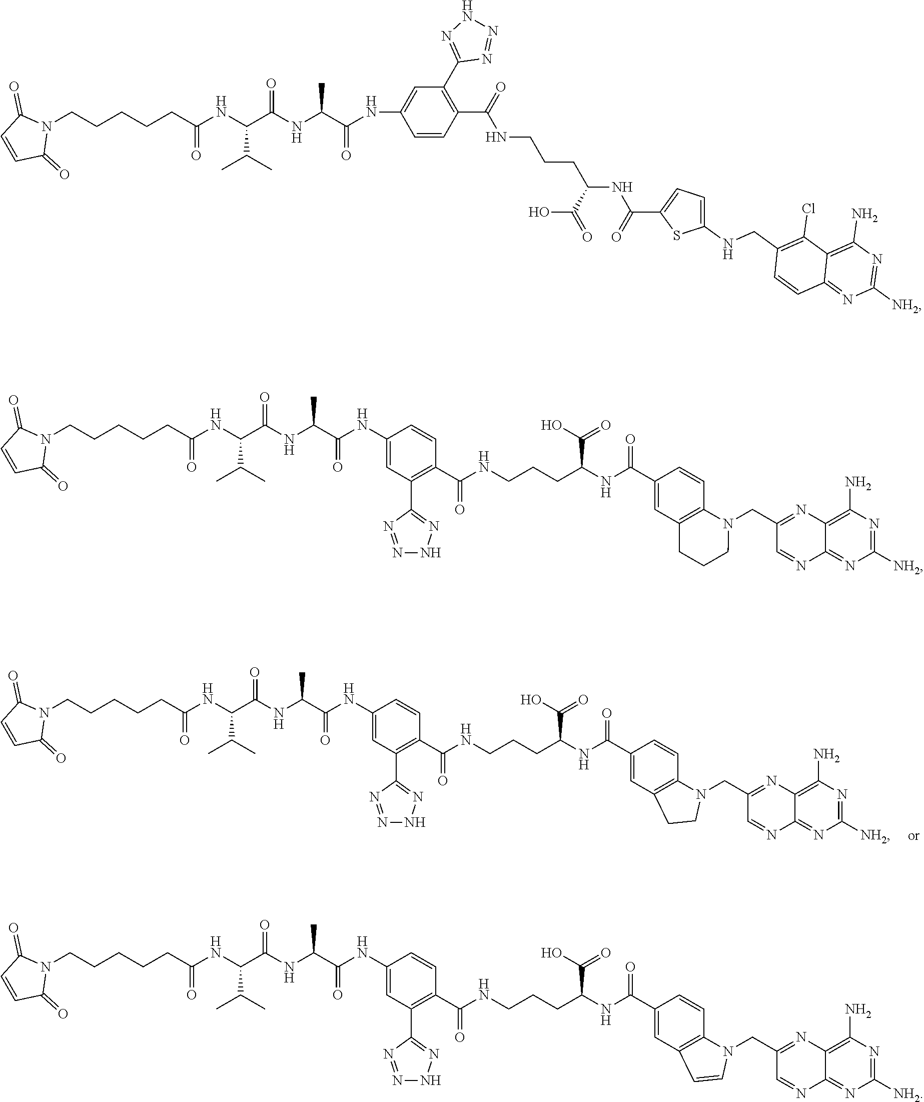

Even more preferred linker-drug compounds according to the invention are

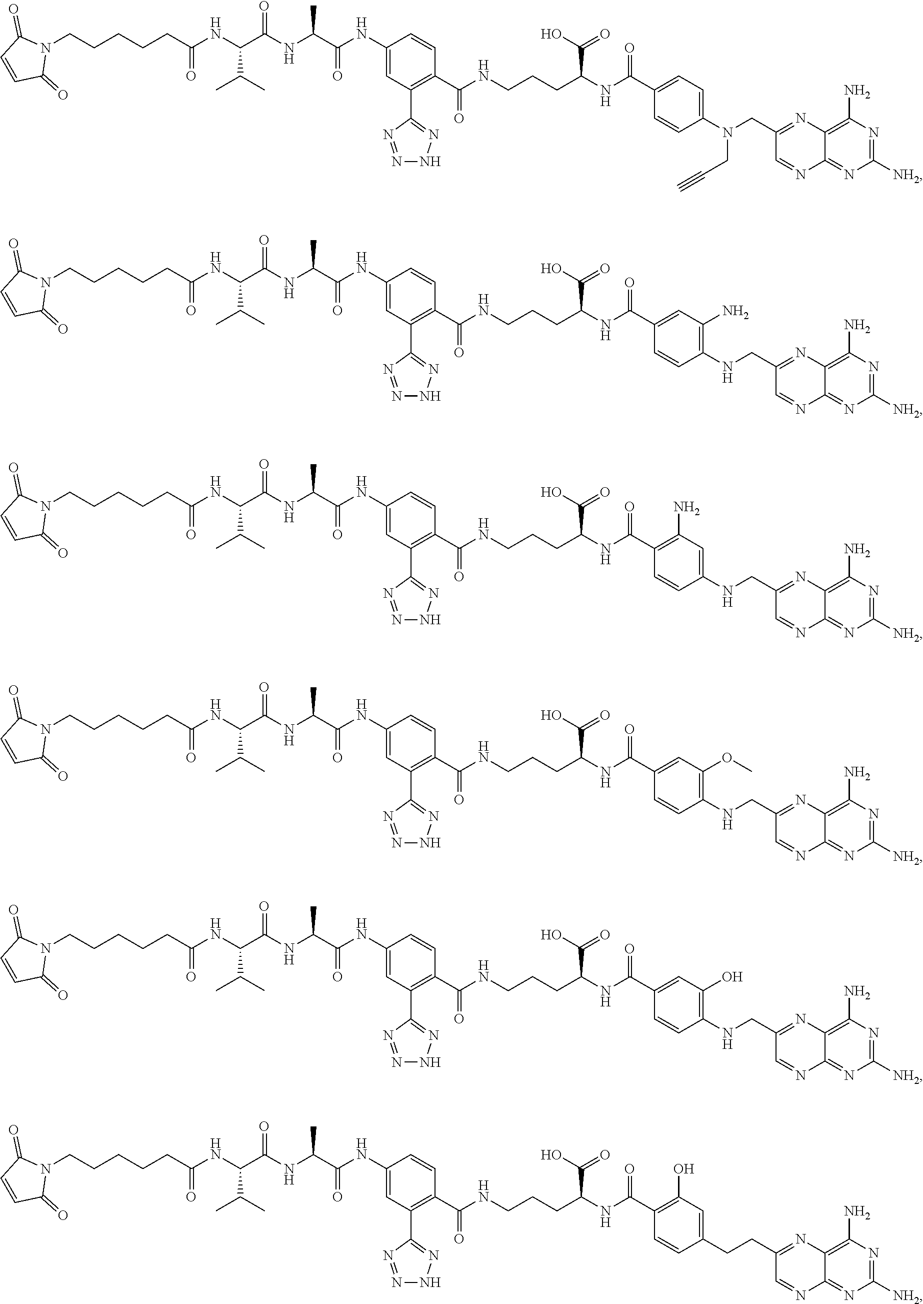

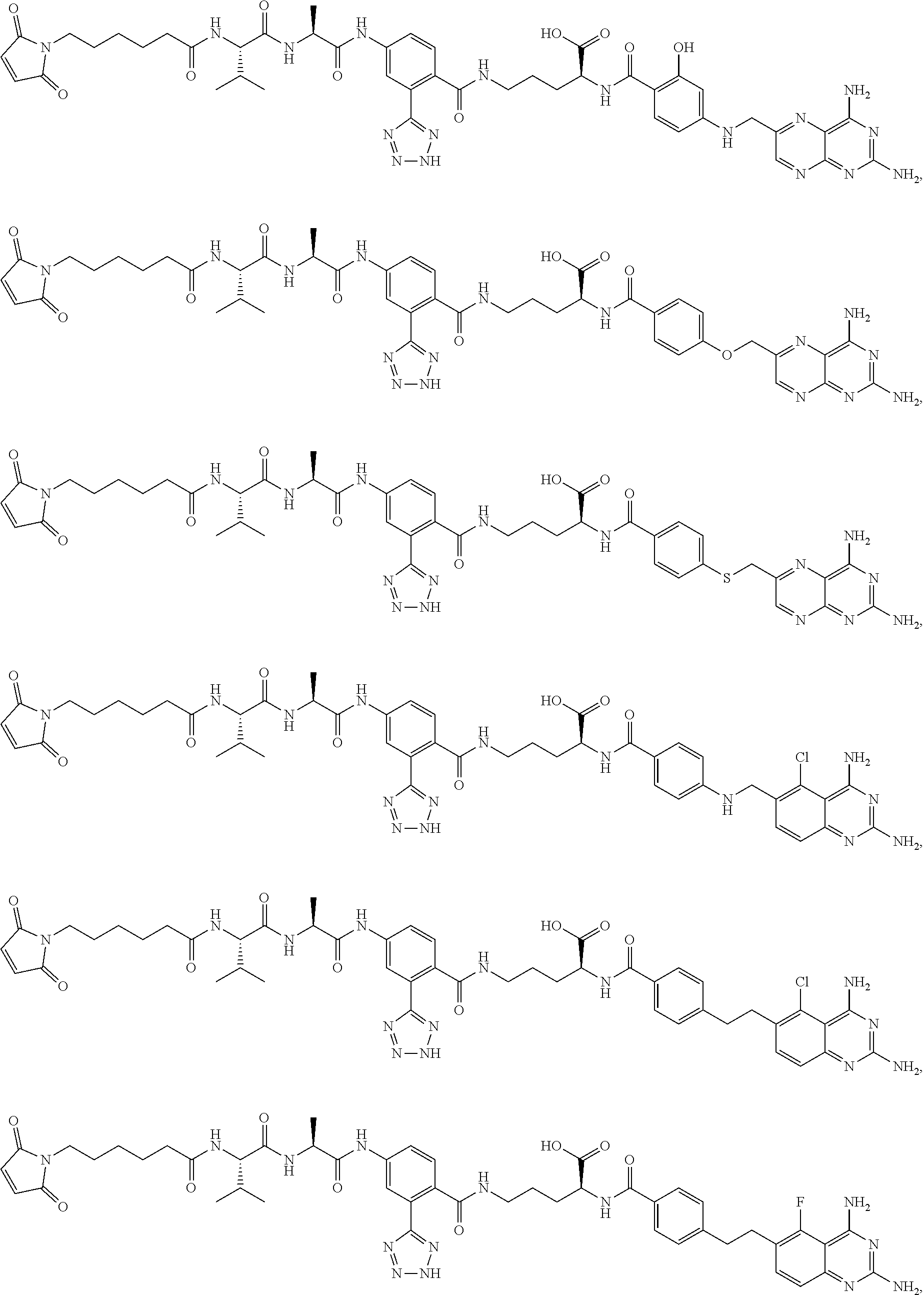

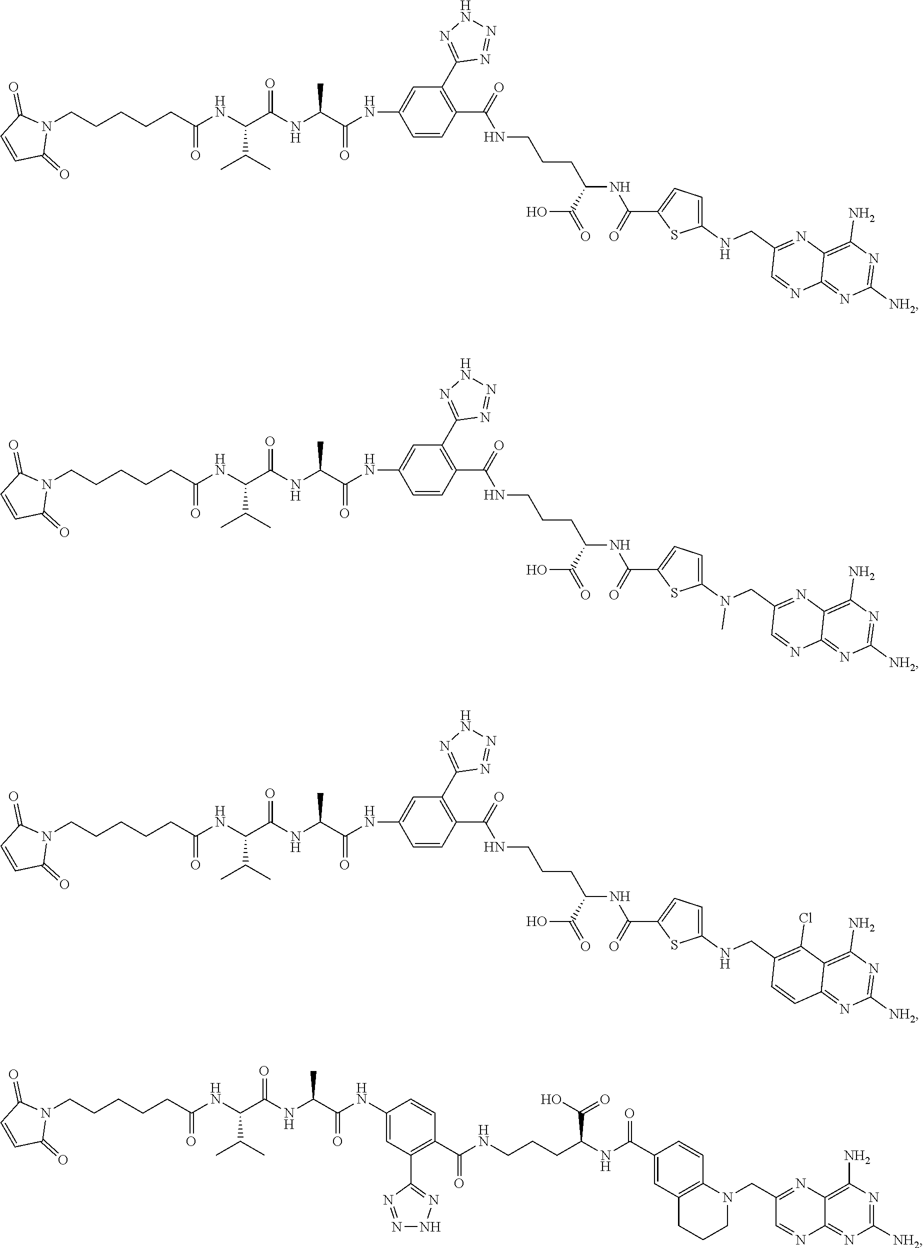

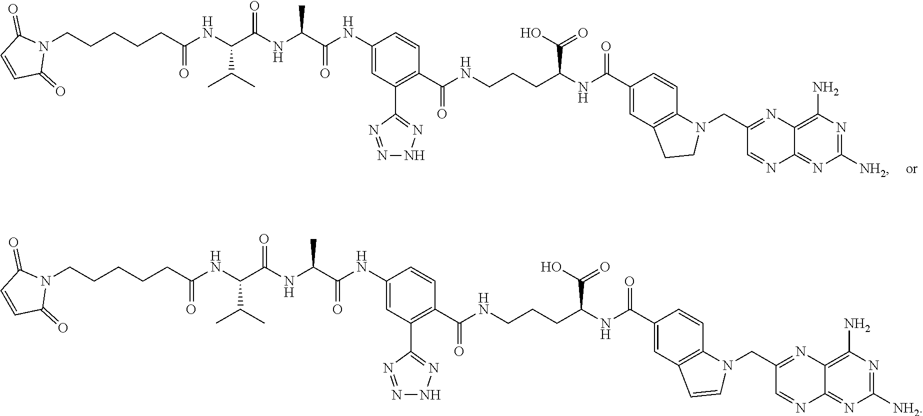 [SMILES: CC(C)[C@H](NC(=O)CCCCCN1C(=O)C=CC1=O)C(=O)N[C@@H](C)C(=O)Nc1ccc(C(=O)NCCC[C@H](NC(=O)c2ccc3c(c2)CCN3Cc2cnc3nc(N)nc(N)c3n2)C(=O)O)c(-c2nn[nH]n2)c1 ; CC(C)[C@H](NC(=O)CCCCCN1C(=O)C=CC1=O)C(=O)N[C@@H](C)C(=O)Nc1ccc(C(=O)NCCC[C@H](NC(=O)c2ccc3c(ccn3Cc3cnc4nc(N)nc(N)c4n3)c2)C(=O)O)c(-c2nn[nH]n2)c1]

Even more preferred linker-drug compounds according to the invention are

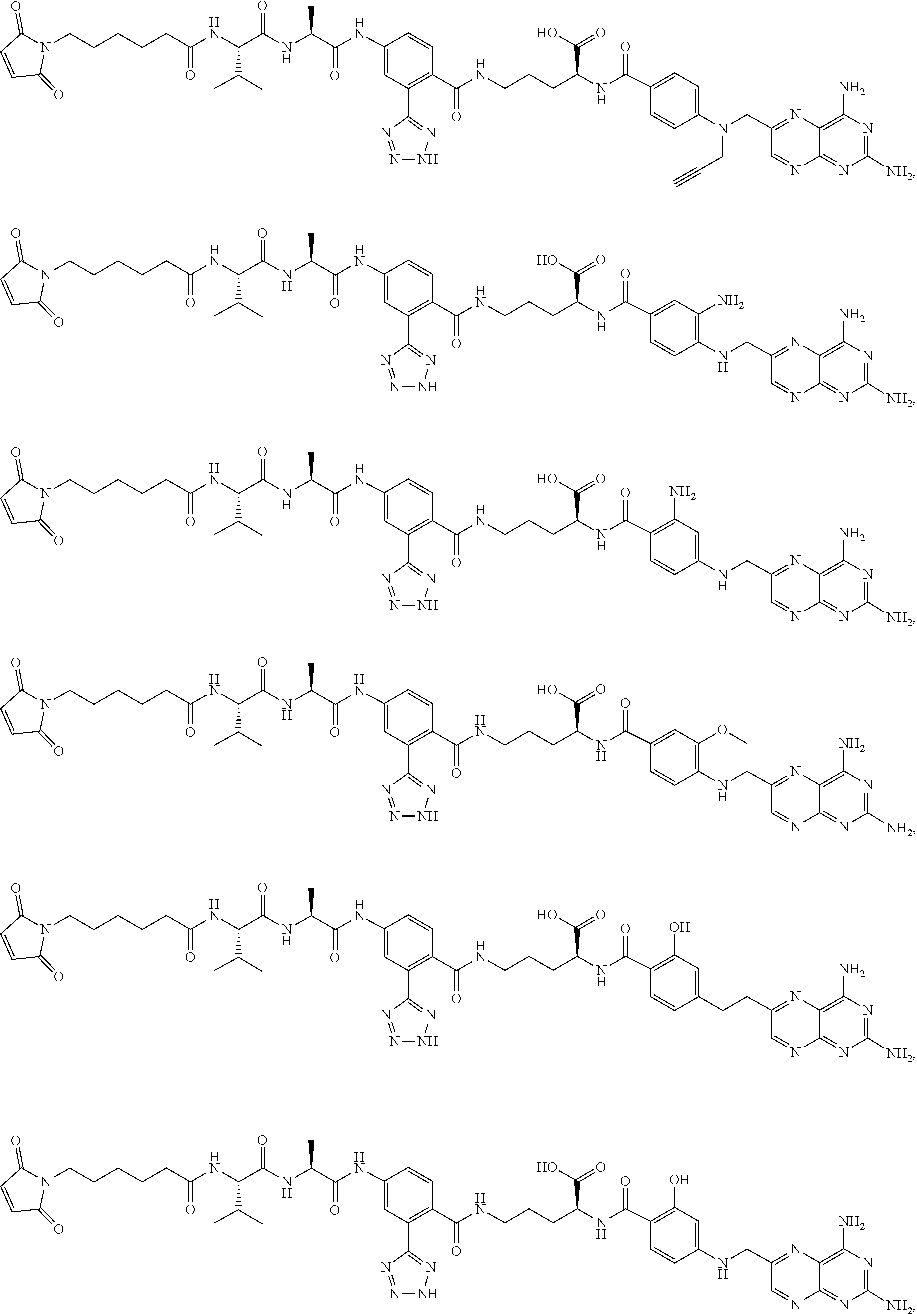

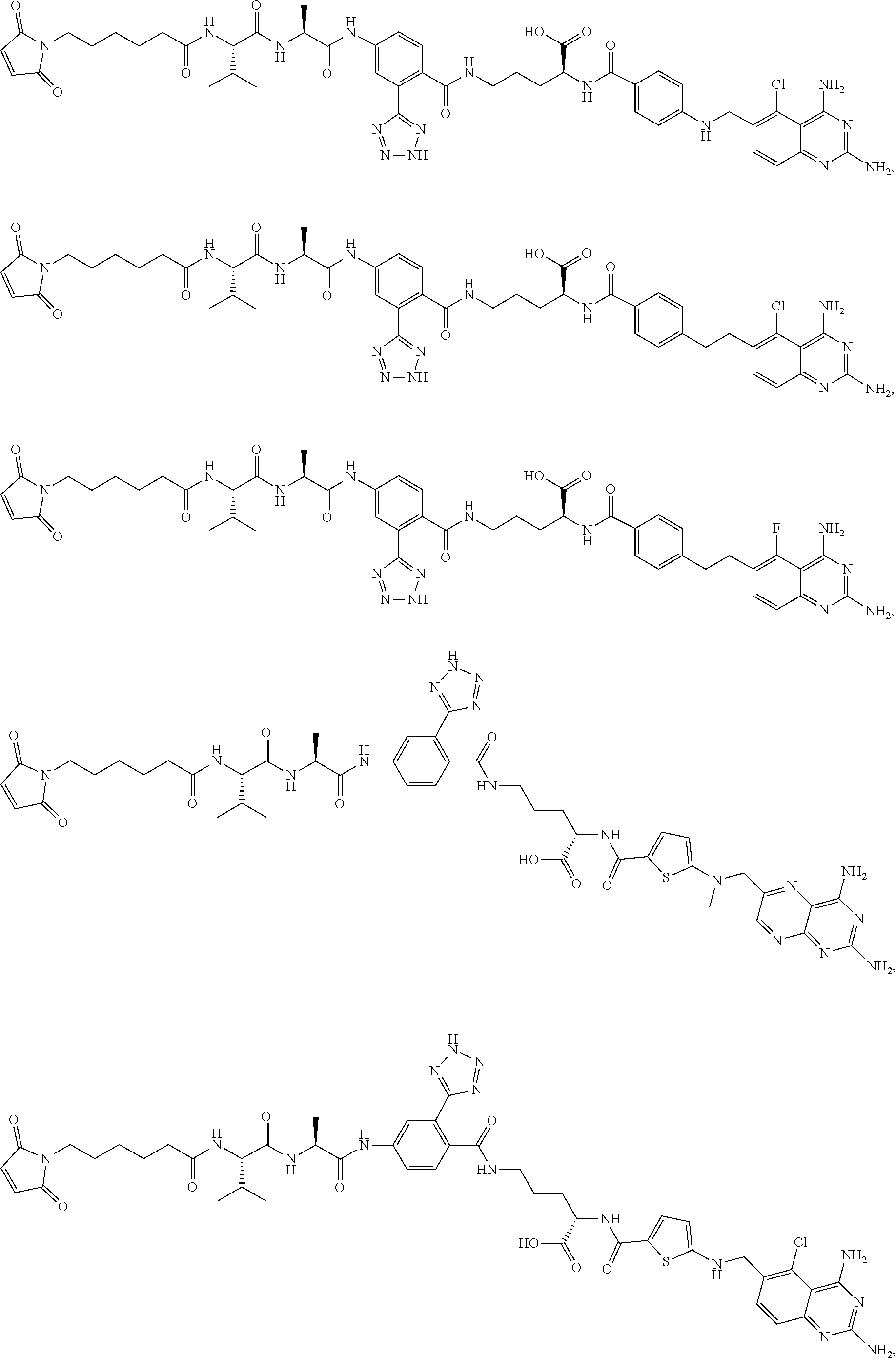

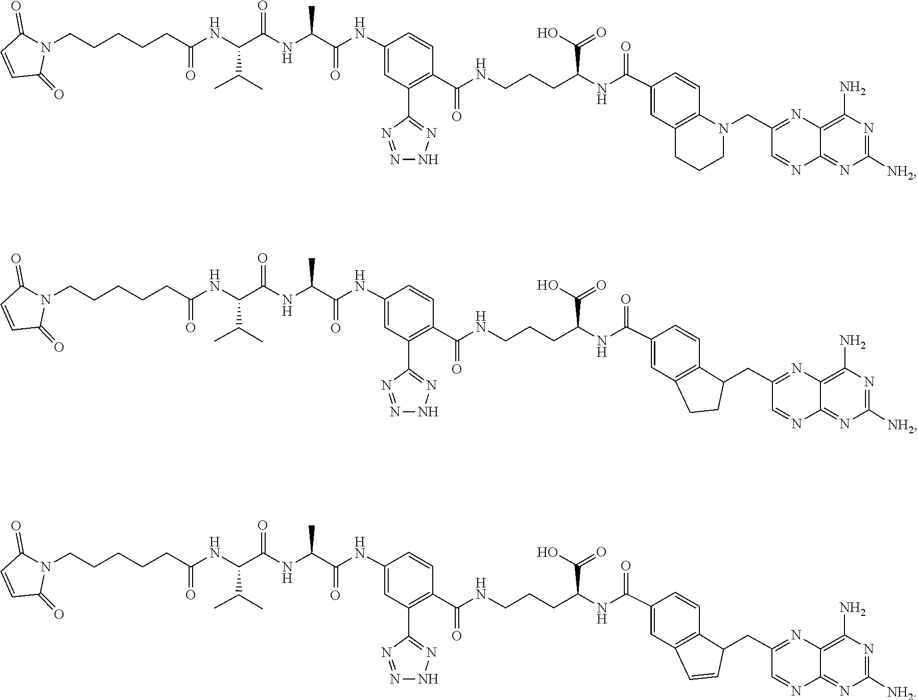 [SMILES: CC(C)[C@H](NC(=O)CCCCCN1C(=O)C=CC1=O)C(=O)N[C@@H](C)C(=O)Nc1ccc(C(=O)NCCC[C@H](NC(=O)c2ccc3c(c2)CCCN3Cc2cnc3nc(N)nc(N)c3n2)C(=O)O)c(-c2nn[nH]n2)c1 ; CC(C)[C@H](NC(=O)CCCCCN1C(=O)C=CC1=O)C(=O)N[C@@H](C)C(=O)Nc1ccc(C(=O)NCCC[C@H](NC(=O)c2ccc3c(c2)CCC3Cc2cnc3nc(N)nc(N)c3n2)C(=O)O)c(-c2nn[nH]n2)c1 ; CC(C)[C@H](NC(=O)CCCCCN1C(=O)C=CC1=O)C(=O)N[C@@H](C)C(=O)Nc1ccc(C(=O)NCCC[C@H](NC(=O)c2ccc3c(c2)C=CC3Cc2cnc3nc(N)nc(N)c3n2)C(=O)O)c(-c2nn[nH]n2)c1]

Most preferably the linker-drug compound according to the invention is of formula

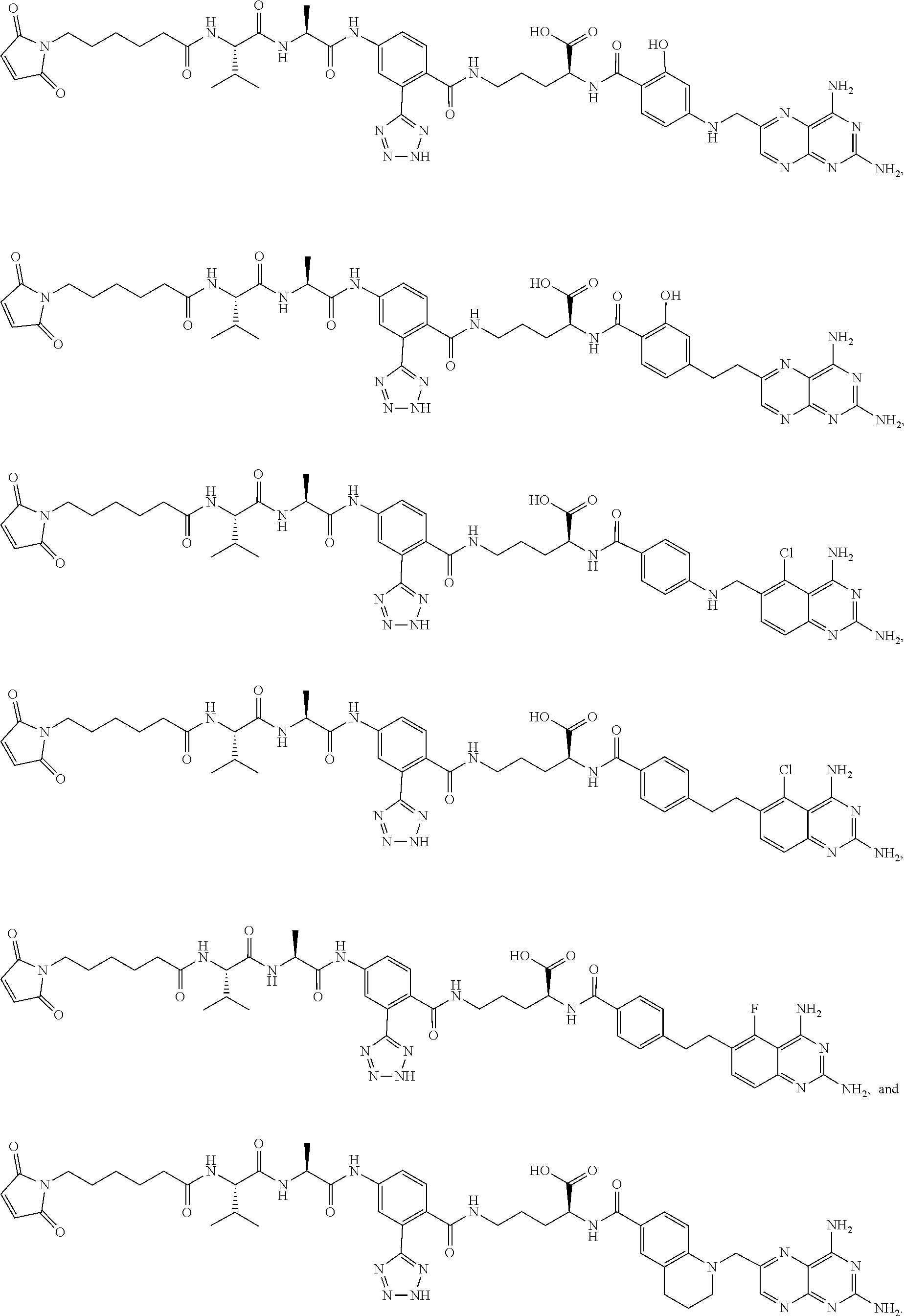

### Processes for Preparing a Linker-Drug Compound According to the Invention

Linker-drug compounds according to the invention may be prepared by the or similar procedures as disclosed in the Examples or by e.g., Rosowsky et al (J. Med. Chem. 1988, 31 1332-1337; J. Med. Chem. 1998, 41 5310-5319; J. Med. Chem. 2000, 43 1620-1634) or Itoh et al (Chem. Pharm. Bull. 2000, 48 1270-1280).

In one aspect, the present invention relates to the use of a compound of formula

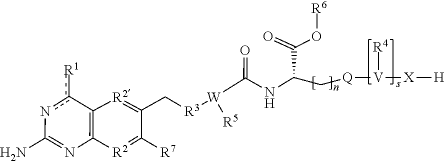

in a process for making a linker-drug compound according to the invention or for making a conjugate comprising the linker-drug compound according to the invention, wherein
R¹ is O, NH₂ or OH;
R² and R^{2′} are independently N, CH, CMe, C-Et, C-ethyn, C-vinyl, C—CN, C—OH, C—OMe, C—SH, C—SMe, C-halogen, wherein halogen is preferably Cl, C—CHal₃, C—CHHal₂, or C—CH₂Hal, wherein Hal is halogen, preferably F;
R³ is O, S, NH, N(C₁₋₅ alkyl), N(C₂₋₄ alkenyl), N(C₂₋₄ alkynyl), N—CH₂CN, N—CH₂CH₂Hal, CH₂, CH(C₁₋₅ alkyl), CH(C₂₋₄ alkenyl), CH(C₂₋₄ alkynyl), CH(C₁₋₄ alkoxyl), CH—CH₂CN, or CH—CH₂CH₂Hal, wherein Hal is halogen, preferably F;
R⁴ is H, halogen, —COOH, OH, NH₂, —CONH₂, —CONHR, —CONR₂, C₁₋₄ alkyl, C₁₋₄ alkoxyl, benzyloxy, tetrazole, —SO₃H, —NSO₃H, —OSO₃H, —PO₃H₂, —NPO₃H₂, —OPO₃H₂, —CN, or azido,
wherein R is selected from H and C₁₋₅ alkyl, or R⁴ is a carboxylic acid bioisostere;
R⁵ is H, halogen, NH₂, OH, SH, CF₃, C₁₋₄ alkyl, C₂₋₄ alkenyl, C₂₋₄ alkynyl, C₁₋₄ alkoxyl, or C₁₋₄ alkylthio, preferably H, F, CH₃, CF₃, CH₂CH₃, CH═CH₂, CH₂CF₃, or CF₂CF₃, more preferably H or F, or R³ and R⁵ are optionally being joined by one or more bonds to form an optionally substituted carbocycle or heterocycle, preferably an optionally substituted 5- or 6-membered carbocycle or heterocycle;
R⁶ is H, C₁₋₄ alkyl, C₂₋₄ alkenyl, C₃₋₆ cycloalkyl, preferably H;
R⁷ is H or F;
n is 1, 2, 3, or 4, preferably 3;
Q is either absent, —N(R⁸)—(C═O)—, —(C═O)—N(R⁸)—, —CH₂N(R⁸)—, —N(R⁸)CH₂—, —N(R⁸)SO₂—, or —SO₂N(R⁸)—, wherein R⁸ is H, C₁₋₄ alkyl, C₁₋₄ alkenyl, or C₁₋₄ alkynyl, preferably H, or Q is an amide bond bioisostere;
V is an aryl, heteroaryl, heterocycle, or cycloalkane that is optionally substituted with one or more independently selected R⁴-groups and is independently selected from

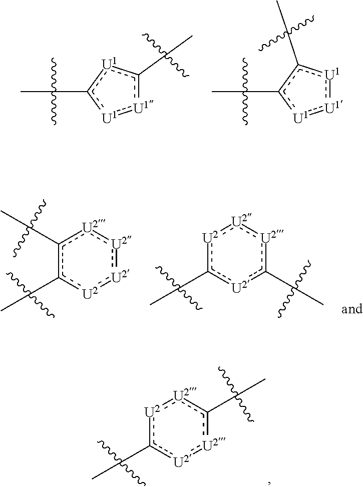

wherein U¹, U^{1″}, U^{1″}, U², U^{2′}, U^{2″}, and U^{2′″} are independently selected from C, CH, S, N, NH, N(C₁₋₅ alkyl), and O, or V is selected from the group consisting of

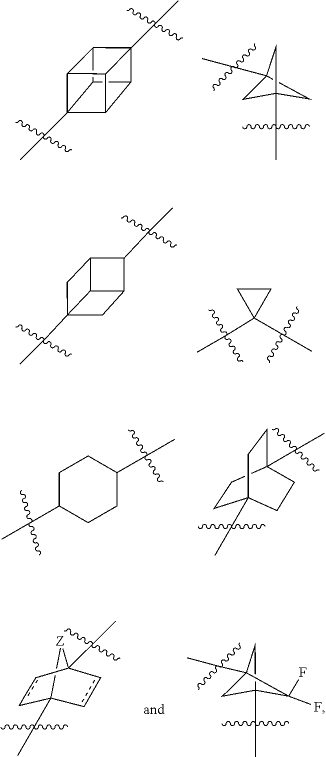

wherein Z is O, S, NH or NR^{c}, and R^{c} is selected from H and C₁₋₅ alkyl;
s is 0 or 1, preferably 1;
W is an aryl or heteroaryl that is optionally substituted with one or more independently selected R⁵-groups;
X is a connecting group selected from O, NH, S, C₁₋₅ alkylene, C₁₋₅ alkenylene, and C₁₋₅ alkynylene, preferably from O and NH, more preferably X is NH; and
[symbol] means that the indicated bond may be a single bond or a non-cumulated, optionally delocalized, double bond.

In one embodiment, the present invention relates to the use of a compound of formula

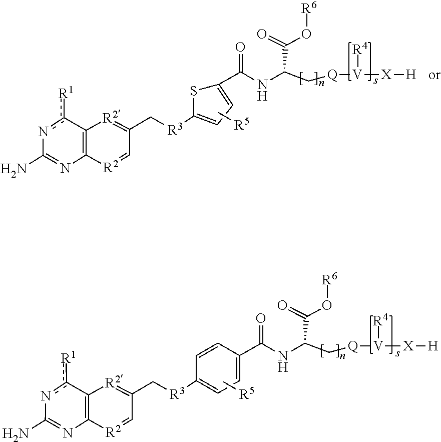

in a process for making a linker-drug compound according to the invention or for making a conjugate comprising the linker-drug compound according to the invention.

In another embodiment, the present invention relates to the use of a compound of formula

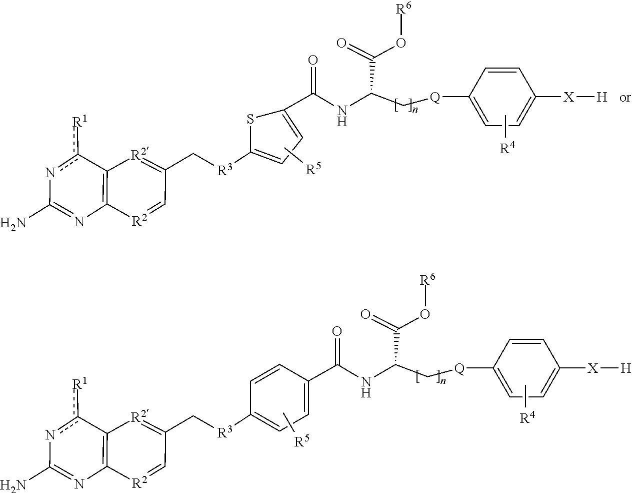

in a process for making a linker-drug compound according to the invention or for making a conjugate comprising the linker-drug compound according to the invention.

In yet another embodiment, the present invention relates to the use of a compound of formula

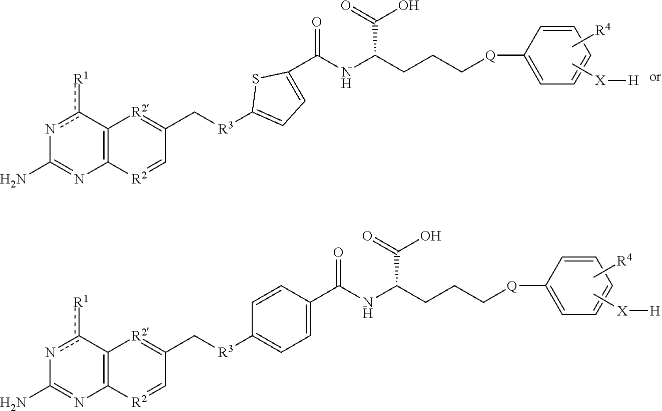

in a process for making a linker-drug compound according to the invention or for making a conjugate comprising the linker-drug compound according to the invention.

In yet another embodiment, the present invention relates to the use of a compound of formula

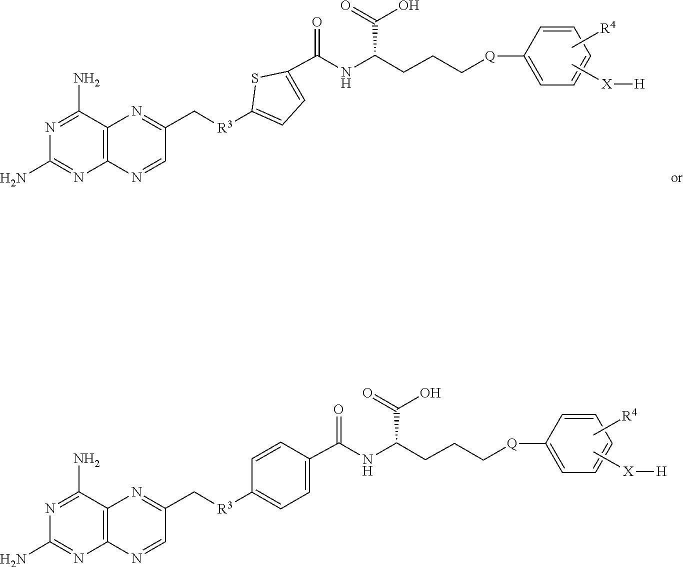

in a process for making a linker-drug compound according to the invention or for making a conjugate comprising the linker-drug compound according to the invention.

In yet another embodiment, the present invention relates to the use of a compound of formula

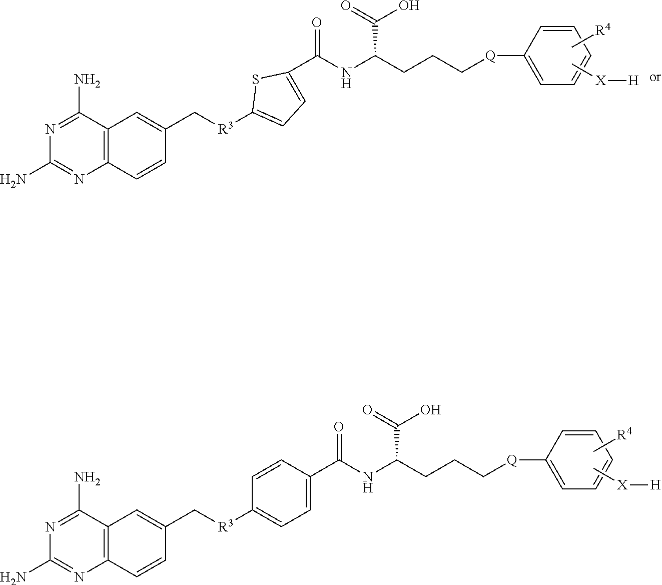

in a process for making a linker-drug compound according to the invention or for making a conjugate comprising the linker-drug compound according to the invention.

In yet another embodiment, the present invention relates to the use of a compound of formula

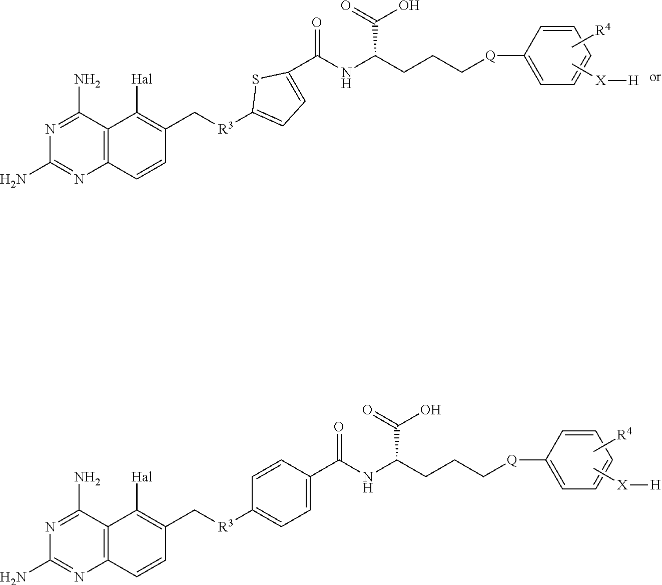

in a process for making a linker-drug compound according to the invention or for making a conjugate comprising the linker-drug compound according to the invention.

In yet another embodiment, the present invention relates to the use of a compound of formula

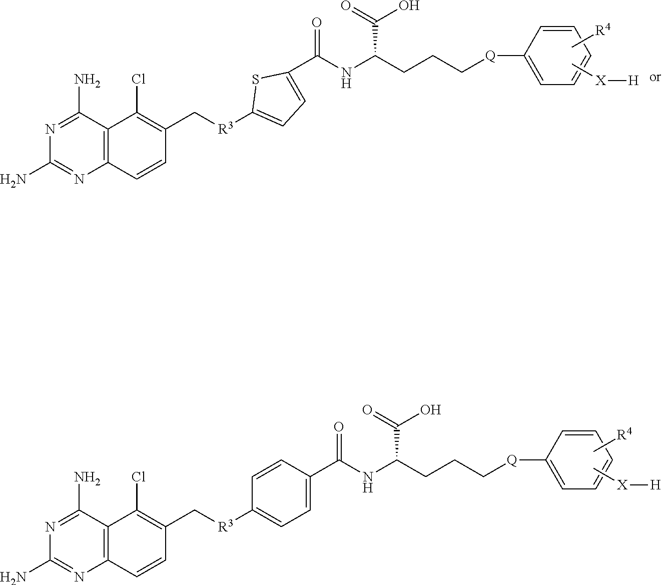

in a process for making a linker-drug compound according to the invention or for making a conjugate comprising the linker-drug compound according to the invention.

In yet another embodiment, the present invention relates to the use of a compound of formula

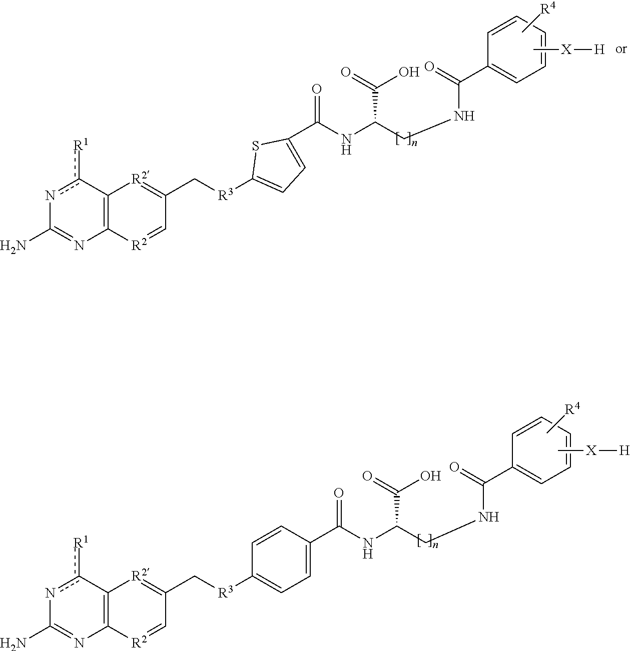

in a process for making a linker-drug compound according to the invention or for making a conjugate comprising the linker-drug compound according to the invention.

In yet another embodiment, the present invention relates to the use of a compound of formula

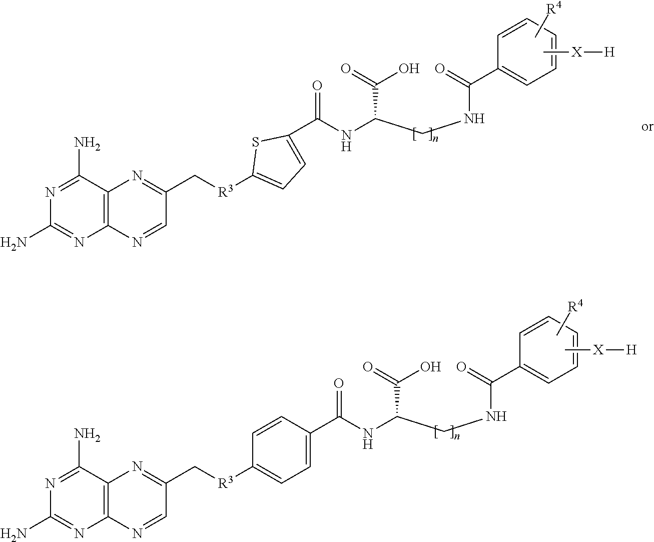

in a process for making a linker-drug compound according to the invention or for making a conjugate comprising the linker-drug compound according to the invention.

In yet another embodiment, the present invention relates to the use of a compound of formula

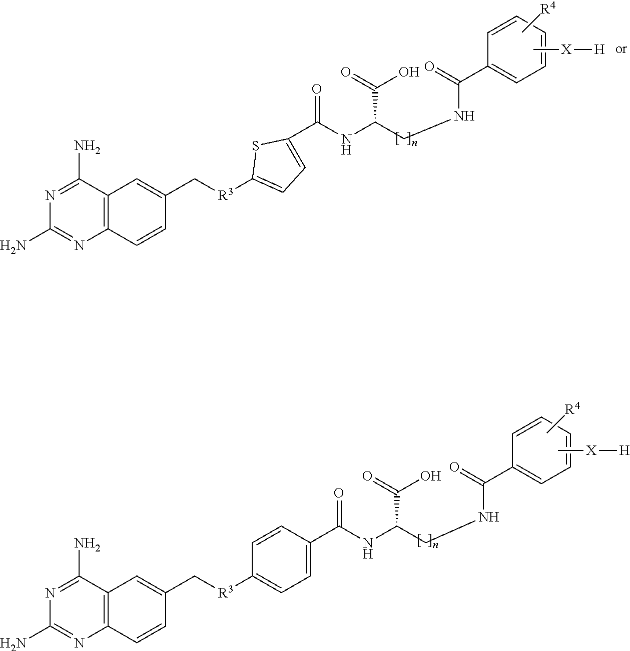

in a process for making a linker-drug compound according to the invention or for making a conjugate comprising the linker-drug compound according to the invention.

In yet another embodiment, the present invention relates to the use of a compound of formula

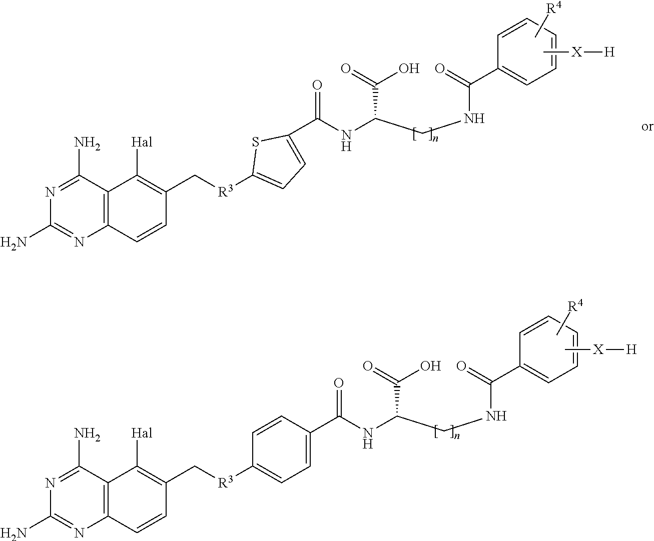

in a process for making a linker-drug compound according to the invention or for making a conjugate comprising the linker-drug compound according to the invention.

In yet another embodiment, the present invention relates to the use of a compound of formula

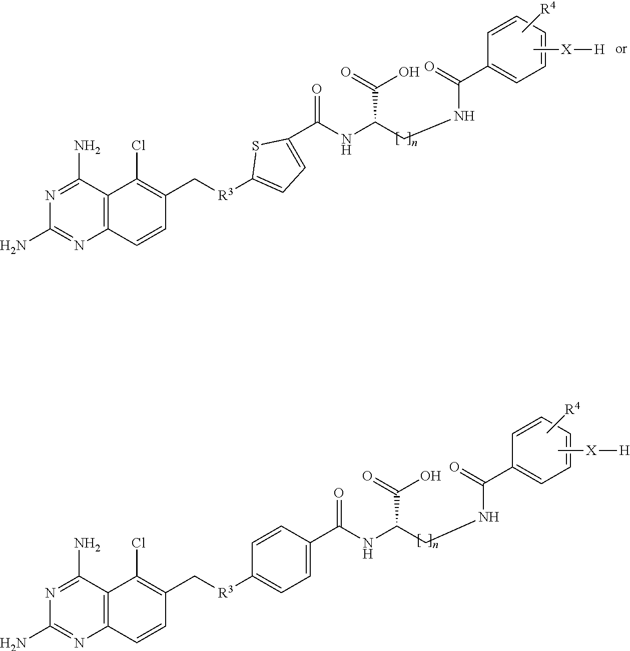

in a process for making a linker-drug compound according to the invention or for making a conjugate comprising the linker-drug compound according to the invention.

In yet another embodiment, the present invention relates to the use of a compound of formula

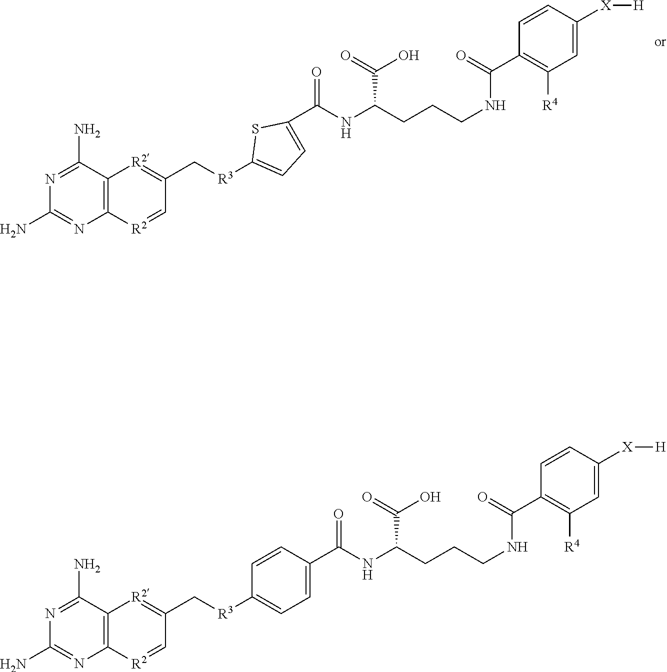

in a process for making a linker-drug compound according to the invention or for making a conjugate comprising the linker-drug compound according to the invention.

In yet another embodiment, the present invention relates to the use of a compound of formula

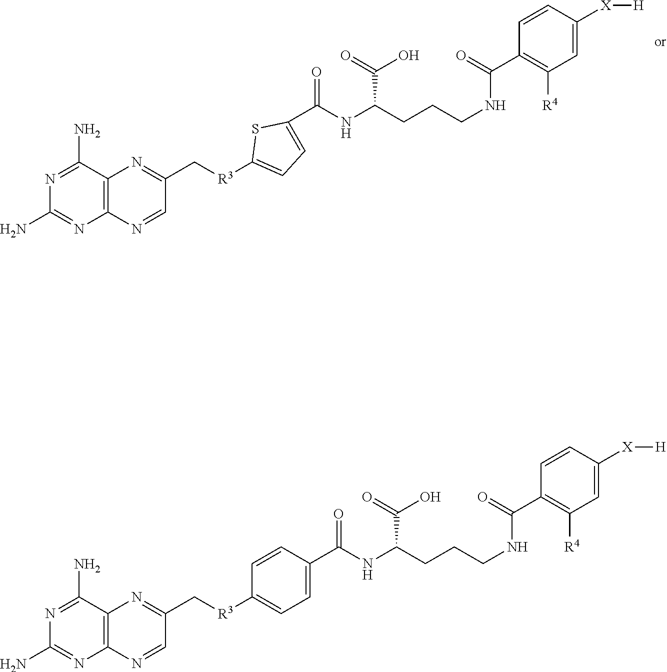

in a process for making a linker-drug compound according to the invention or for making a conjugate comprising the linker-drug compound according to the invention.

In yet another embodiment, the present invention relates to the use of a compound of formula

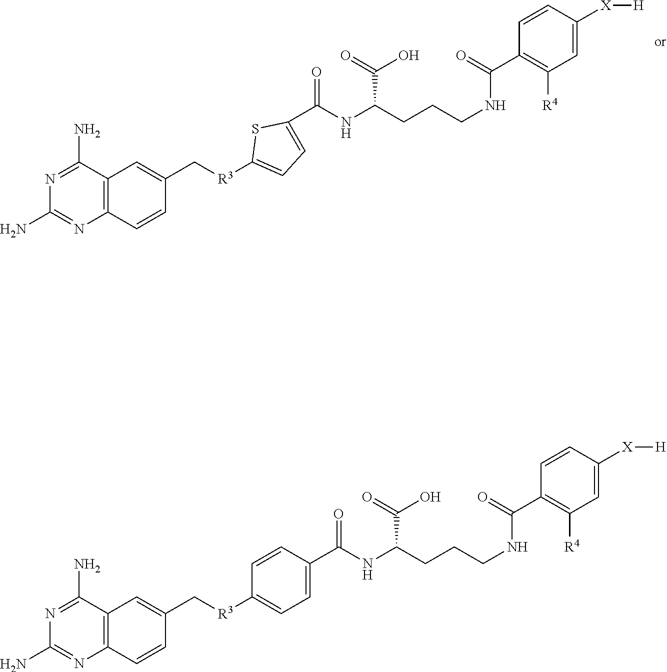

in a process for making a linker-drug compound according to the invention or for making a conjugate comprising the linker-drug compound according to the invention.

In yet another embodiment, the present invention relates to the use of a compound of formula

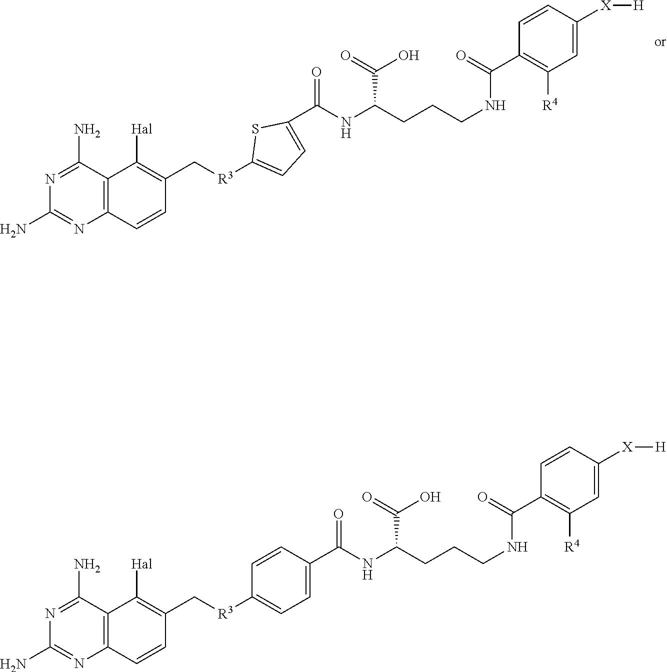

in a process for making a linker-drug compound according to the invention or for making a conjugate comprising the linker-drug compound according to the invention.

In yet another embodiment, the present invention relates to the use of a compound of formula

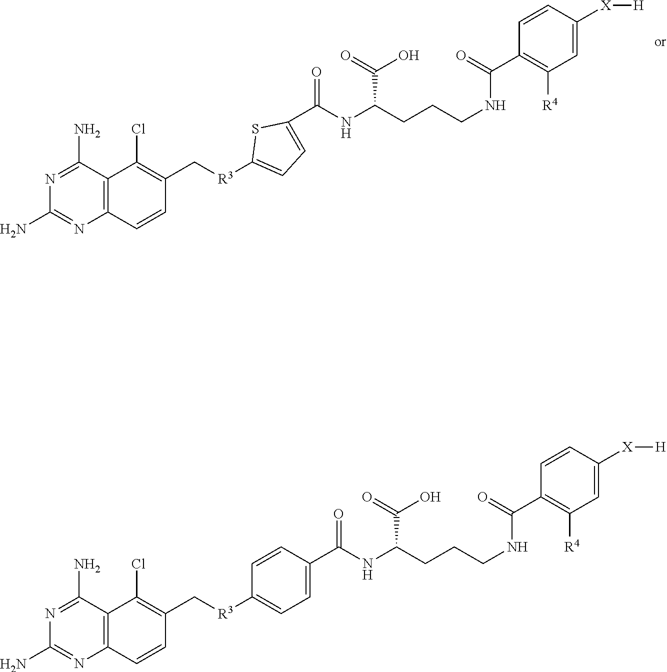

in a process for making a linker-drug compound according to the invention or for making a conjugate comprising the linker-drug compound according to the invention.

In yet another embodiment, the present invention relates to the use of a compound of formula

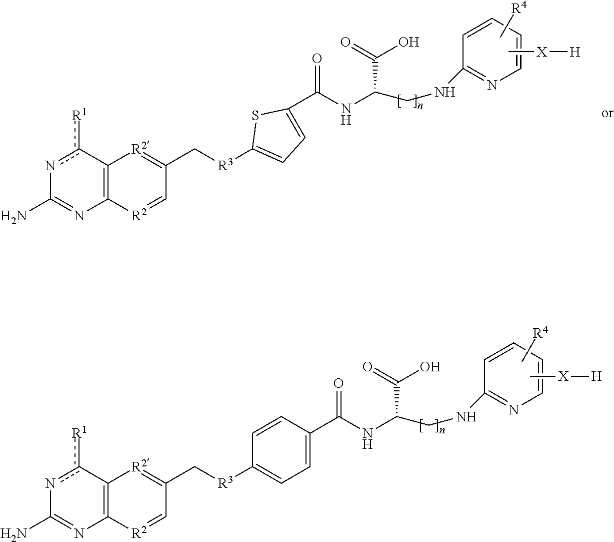

in a process for making a linker-drug compound according to the invention or for making a conjugate comprising the linker-drug compound according to the invention.

In yet another embodiment, the present invention relates to the use of a compound of formula

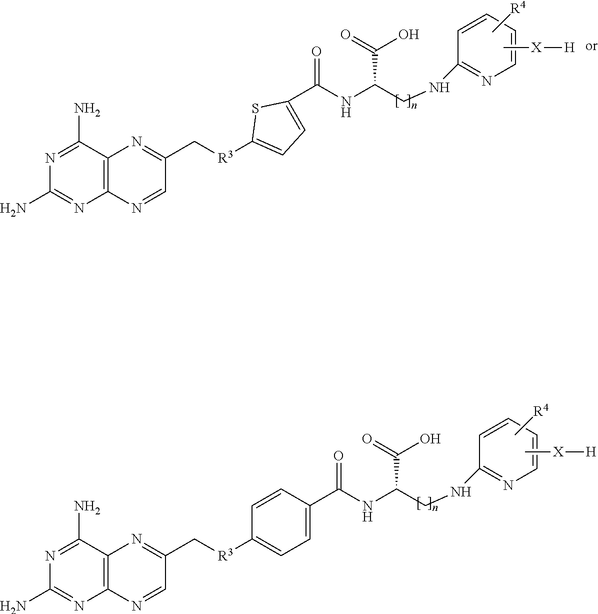

in a process for making a linker-drug compound according to the invention or for making a conjugate comprising the linker-drug compound according to the invention.

In yet another embodiment, the present invention relates to the use of a compound of

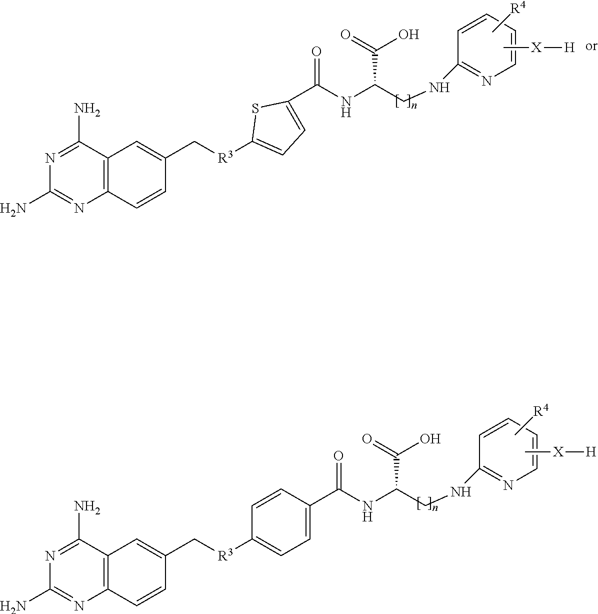

in a process for making a linker-drug compound according to the invention or for making a conjugate comprising the linker-drug compound according to the invention.

In yet another embodiment, the present invention relates to the use of a compound of formula

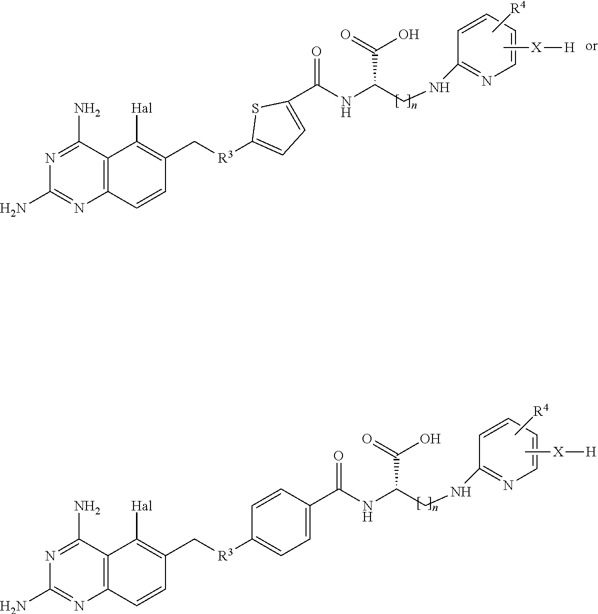

in a process for making a linker-drug compound according to the invention or for making a conjugate comprising the linker-drug compound according to the invention.

In yet another embodiment, the present invention relates to the use of a compound of formula

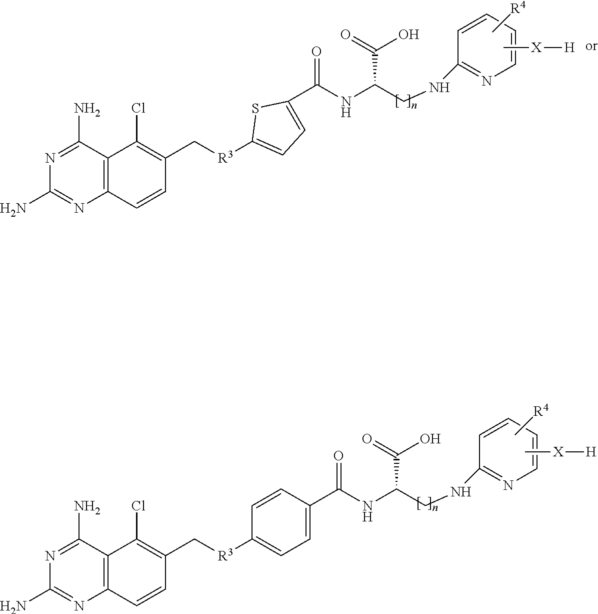

in a process for making a linker-drug compound according to the invention or for making a conjugate comprising the linker-drug compound according to the invention.

In a specific embodiment, the present invention relates to the use of a compound of formula

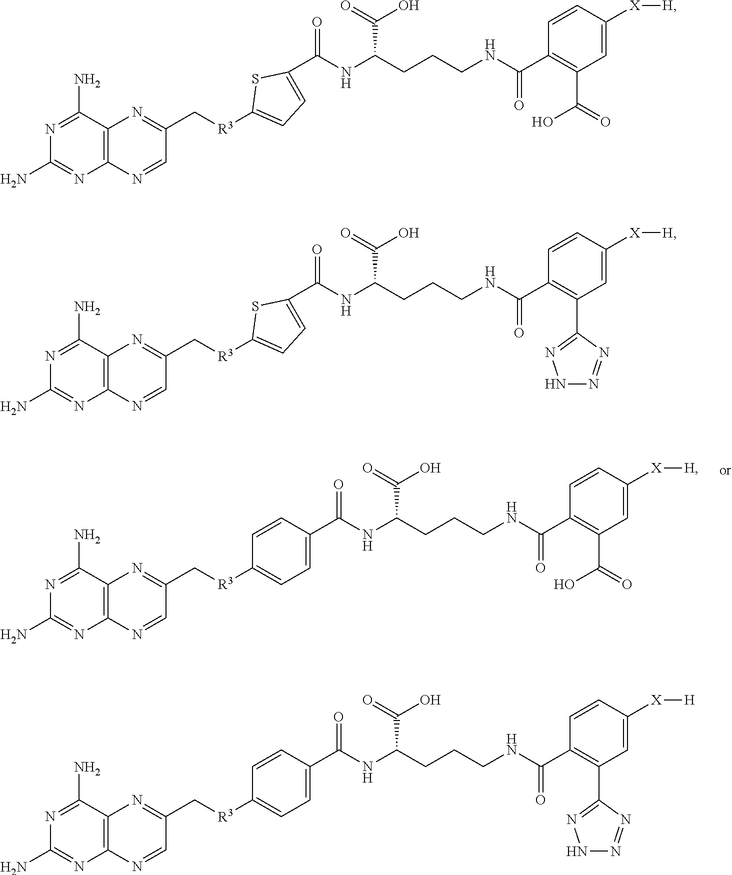

in a process for making a linker-drug compound according to the invention or for making a conjugate comprising the linker-drug compound according to the invention.

In another specific embodiment, the present invention relates to the use of a compound of formula

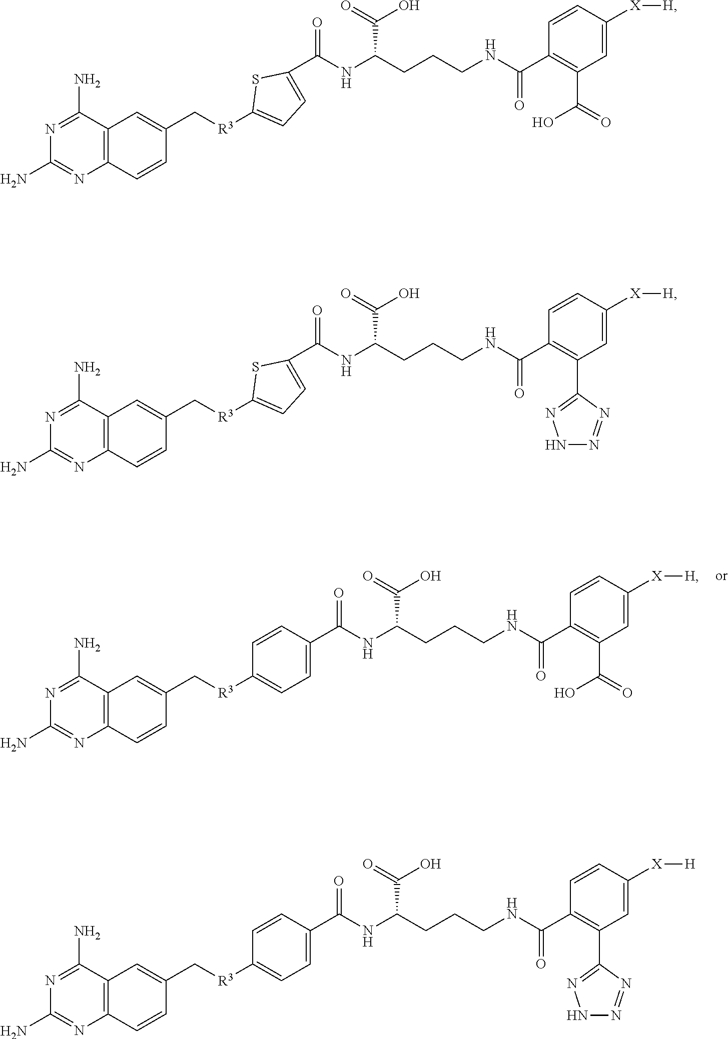

in a process for making a linker-drug compound according to the invention or for making a conjugate comprising the linker-drug compound according to the invention.

In another specific embodiment, the present invention relates to the use of a compound of formula

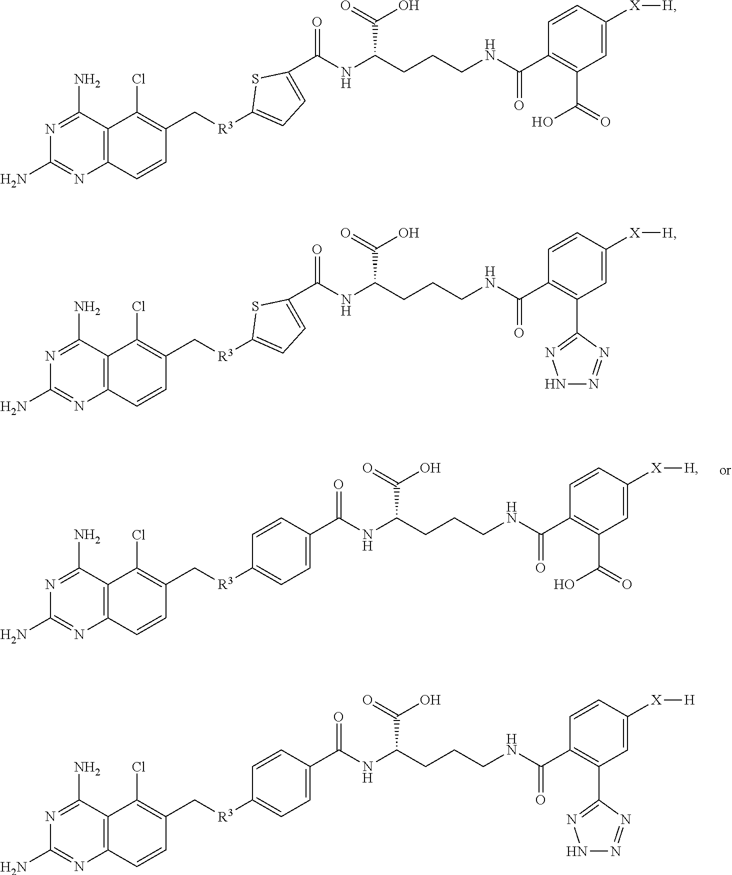

in a process for making a linker-drug compound according to the invention or for making a conjugate comprising the linker-drug compound according to the invention.

Preferably, the compound is of formula

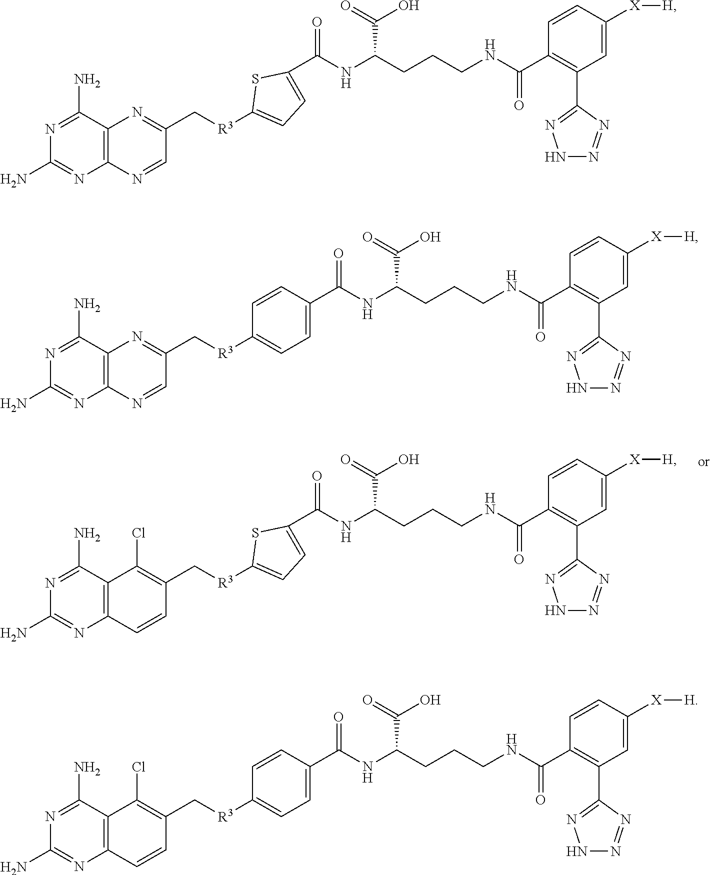

More preferably, the compound is of formula

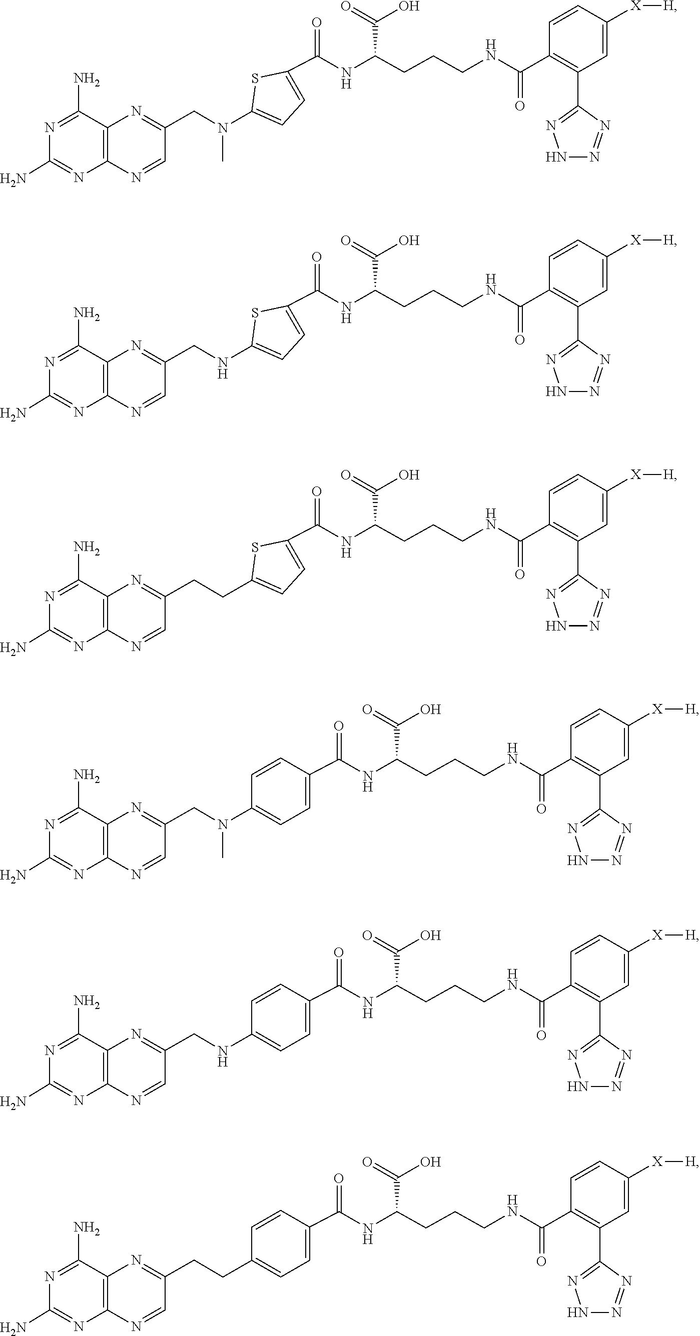

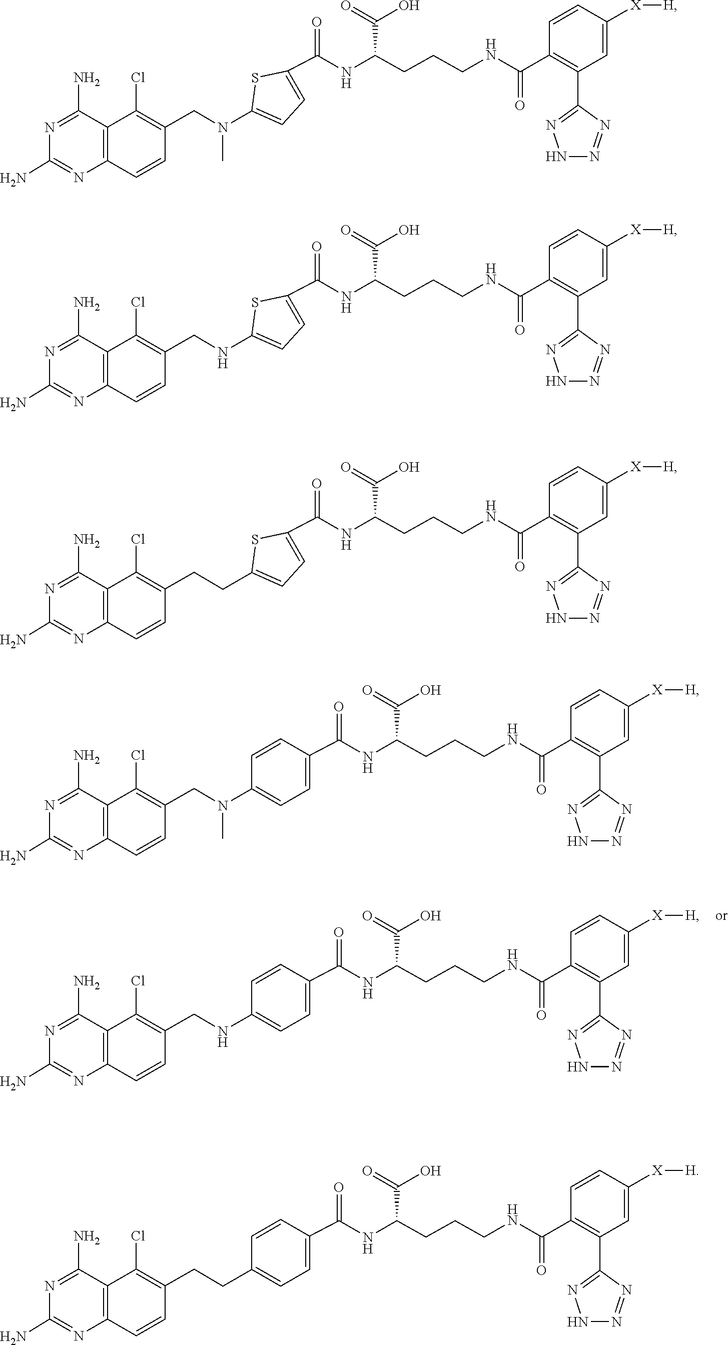

Most preferably, the compound is of formula

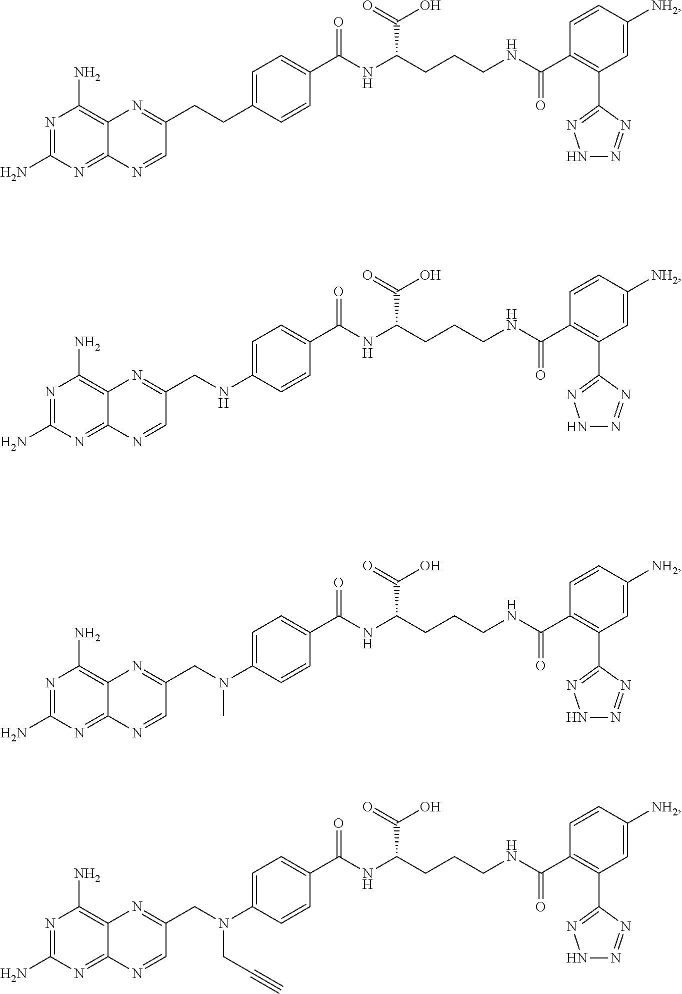 [SMILES: Nc1ccc(C(=O)NCCC[C@H](NC(=O)c2ccc(CCc3cnc4nc(N)nc(N)c4n3)cc2)C(=O)O)c(-c2nn[nH]n2)c1 ; Nc1ccc(C(=O)NCCC[C@H](NC(=O)c2ccc(NCc3cnc4nc(N)nc(N)c4n3)cc2)C(=O)O)c(-c2nn[nH]n2)c1 ; CN(Cc1cnc2nc(N)nc(N)c2n1)c1ccc(C(=O)N[C@@H](CCCNC(=O)c2ccc(N)cc2-c2nn[nH]n2)C(=O)O)cc1 ; C#CCN(Cc1cnc2nc(N)nc(N)c2n1)c1ccc(C(=O)N[C@@H](CCCNC(=O)c2ccc(N)cc2-c2nn[nH]n2)C(=O)O)cc1]

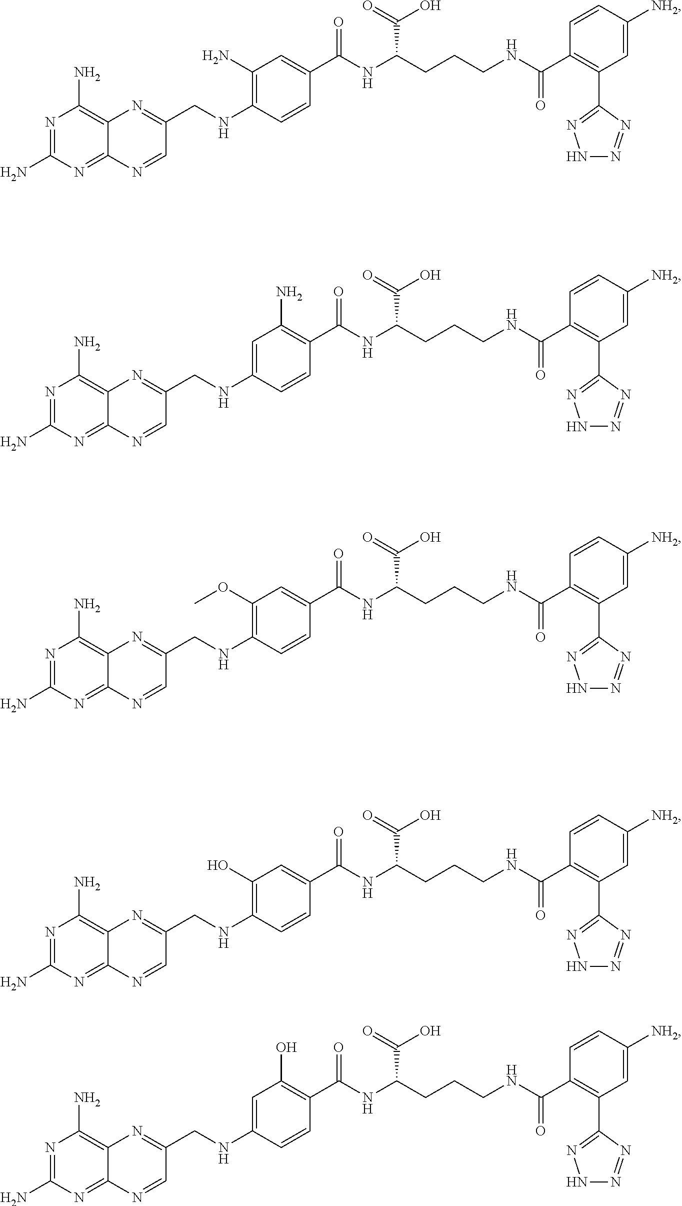 [SMILES: Nc1ccc(C(=O)NCCC[C@H](NC(=O)c2ccc(NCc3cnc4nc(N)nc(N)c4n3)c(N)c2)C(=O)O)c(-c2nn[nH]n2)c1 ; Nc1ccc(C(=O)NCCC[C@H](NC(=O)c2ccc(NCc3cnc4nc(N)nc(N)c4n3)cc2N)C(=O)O)c(-c2nn[nH]n2)c1 ; COc1cc(C(=O)N[C@@H](CCCNC(=O)c2ccc(N)cc2-c2nn[nH]n2)C(=O)O)ccc1NCc1cnc2nc(N)nc(N)c2n1 ; Nc1ccc(C(=O)NCCC[C@H](NC(=O)c2ccc(NCc3cnc4nc(N)nc(N)c4n3)c(O)c2)C(=O)O)c(-c2nn[nH]n2)c1 ; Nc1ccc(C(=O)NCCC[C@H](NC(=O)c2ccc(NCc3cnc4nc(N)nc(N)c4n3)cc2O)C(=O)O)c(-c2nn[nH]n2)c1]

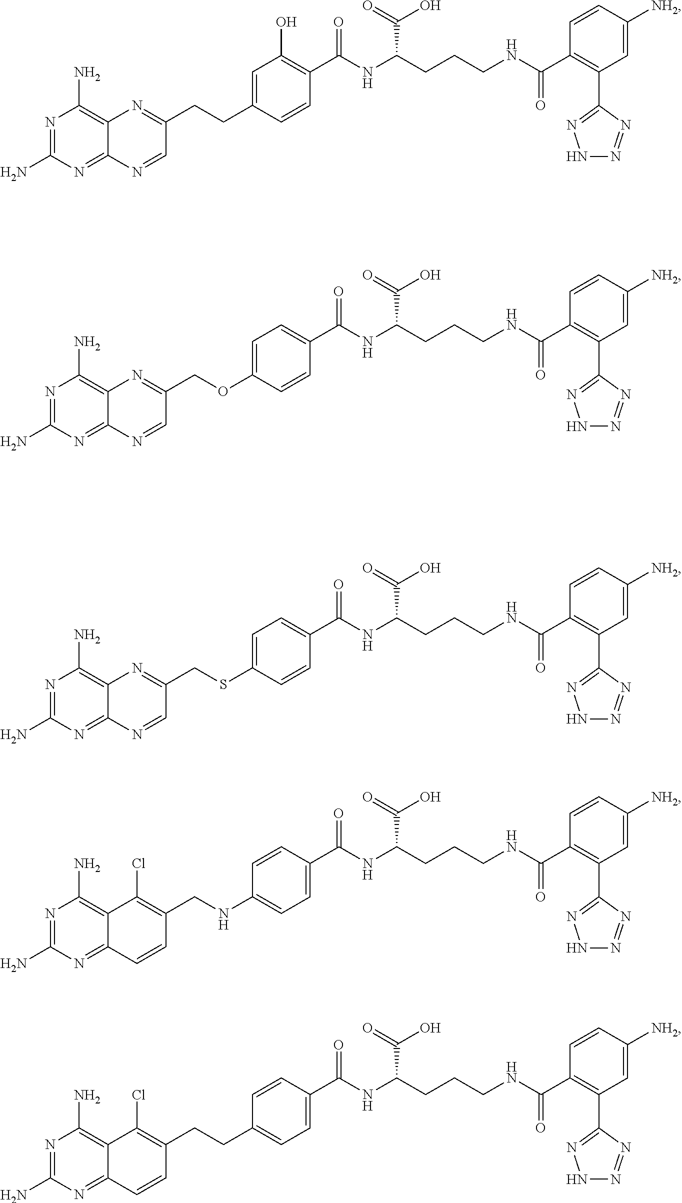 [SMILES: Nc1ccc(C(=O)NCCC[C@H](NC(=O)c2ccc(CCc3cnc4nc(N)nc(N)c4n3)cc2O)C(=O)O)c(-c2nn[nH]n2)c1 ; Nc1ccc(C(=O)NCCC[C@H](NC(=O)c2ccc(OCc3cnc4nc(N)nc(N)c4n3)cc2)C(=O)O)c(-c2nn[nH]n2)c1 ; Nc1ccc(C(=O)NCCC[C@H](NC(=O)c2ccc(SCc3cnc4nc(N)nc(N)c4n3)cc2)C(=O)O)c(-c2nn[nH]n2)c1 ; Nc1ccc(C(=O)NCCC[C@H](NC(=O)c2ccc(NCc3ccc4nc(N)nc(N)c4c3Cl)cc2)C(=O)O)c(-c2nn[nH]n2)c1 ; Nc1ccc(C(=O)NCCC[C@H](NC(=O)c2ccc(CCc3ccc4nc(N)nc(N)c4c3Cl)cc2)C(=O)O)c(-c2nn[nH]n2)c1]

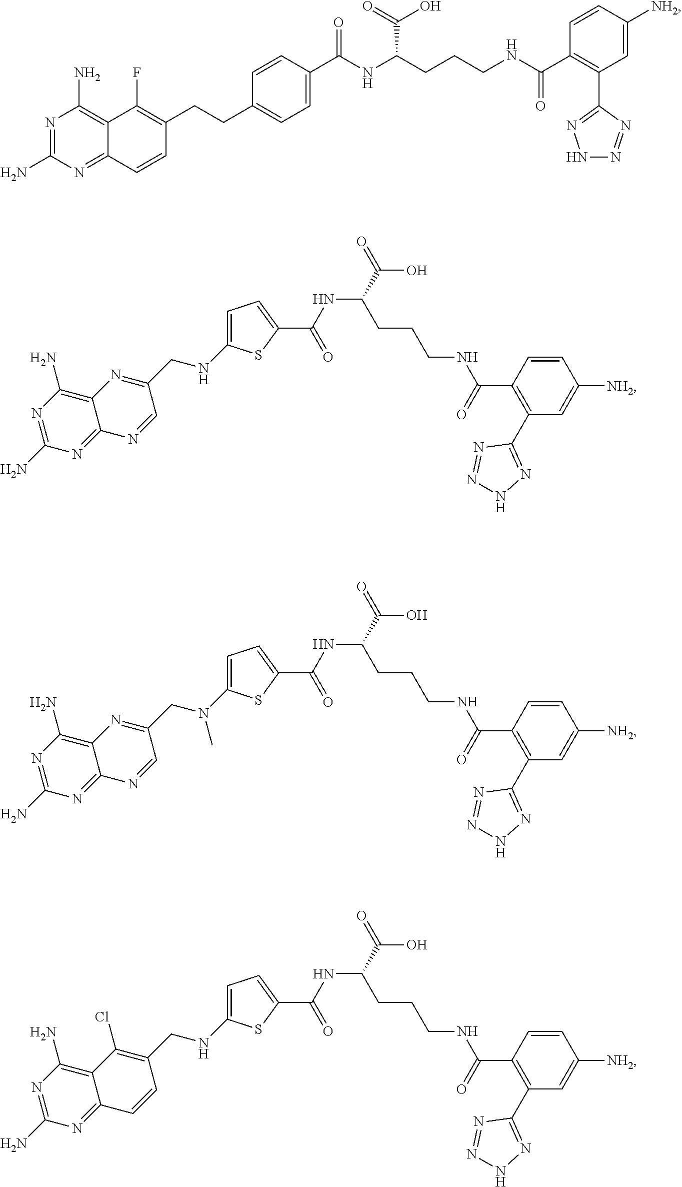 [SMILES: Nc1ccc(C(=O)NCCC[C@H](NC(=O)c2ccc(CCc3ccc4nc(N)nc(N)c4c3F)cc2)C(=O)O)c(-c2nn[nH]n2)c1 ; Nc1ccc(C(=O)NCCC[C@H](NC(=O)c2ccc(NCc3cnc4nc(N)nc(N)c4n3)s2)C(=O)O)c(-c2nn[nH]n2)c1 ; CN(Cc1cnc2nc(N)nc(N)c2n1)c1ccc(C(=O)N[C@@H](CCCNC(=O)c2ccc(N)cc2-c2nn[nH]n2)C(=O)O)s1 ; Nc1ccc(C(=O)NCCC[C@H](NC(=O)c2ccc(NCc3ccc4nc(N)nc(N)c4c3Cl)s2)C(=O)O)c(-c2nn[nH]n2)c1]

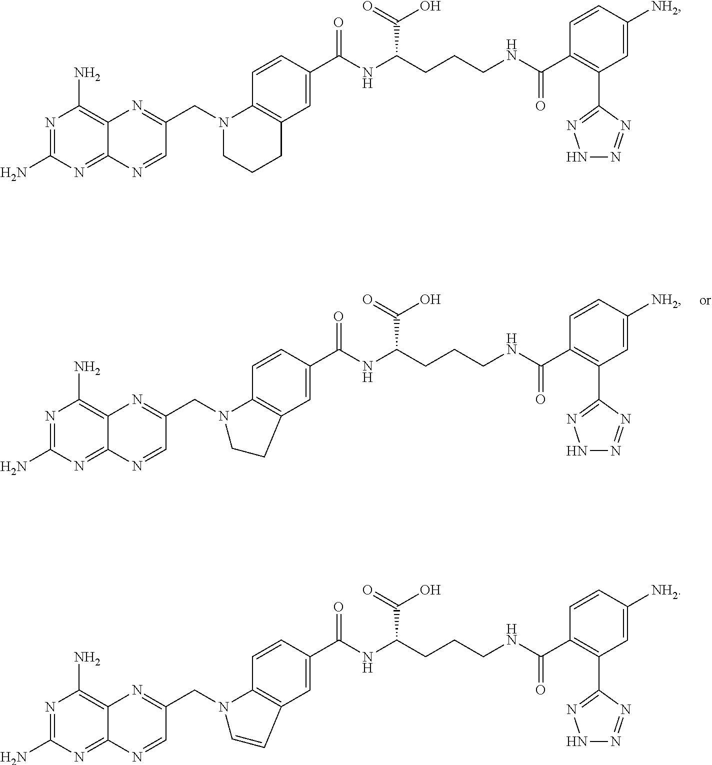 [SMILES: Nc1ccc(C(=O)NCCC[C@H](NC(=O)c2ccc3c(c2)CCCN3Cc2cnc3nc(N)nc(N)c3n2)C(=O)O)c(-c2nn[nH]n2)c1 ; Nc1ccc(C(=O)NCCC[C@H](NC(=O)c2ccc3c(c2)CCN3Cc2cnc3nc(N)nc(N)c3n2)C(=O)O)c(-c2nn[nH]n2)c1 ; Nc1ccc(C(=O)NCCC[C@H](NC(=O)c2ccc3c(ccn3Cc3cnc4nc(N)nc(N)c4n3)c2)C(=O)O)c(-c2nn[nH]n2)c1]

### Inhibitor Conjugates of Polypeptides and Antifolate Linker-Drug Compounds

The invention further provides an inhibitor conjugate, which comprises a linker-drug compound according to the invention conjugated to a further substance, such as for example a polypeptide or a polynucleotide (to form an aptamer). Preferably, the further substance is a polypeptide. More preferably, the polypeptide is an antibody, an antigen-binding fragment thereof, or another targeting molecule. Such inhibitor conjugate is referred to hereinafter as an inhibitor conjugate according to the invention.

When conjugated to a polypeptide, the term “inhibitor conjugate according to the invention” as used throughout the present description refers to a polypeptide to which one or more linker-drug compounds according to the invention are conjugated, i.e., a polypeptide to which one or more linker-drug compounds of general formula (I) are conjugated.

Typically, an inhibitor conjugate according to the invention comprises a polypeptide that binds, reactively associates, or complexes with a receptor, a receptor complex, an antigen, an enzyme, or another moiety associated with an aberrant or malignant cell population, but preferably not or almost not associated with a healthy cell population. The polypeptide in the inhibitor conjugate according to the invention serves as a means to target the linker-drug compound according to the invention to the aberrant or malignant cell population. Suitable polypeptides include antibodies, antigen-binding fragments thereof, enzyme inhibitors, enzyme substrates, receptor ligands, and fusion proteins.

A linker-drug compound according to the invention may be conjugated to a suitable polypeptide via a reactive native amino acid residue present in the suitable polypeptide, e.g., a lysine or a cysteine, or via an N-terminus or C-terminus. Alternatively, a reactive amino acid residue, natural or non-natural, may be genetically engineered into the suitable polypeptide, or may be introduced via post-translational modification. Additionally, if the suitable polypeptide is a glycoprotein, a linker-drug compound according to the invention may be conjugated to the glycoprotein through existing glycans.

It is to be understood that a linker-drug compound according to the invention when comprised in an inhibitor conjugate according to the invention may lack certain atoms or groups of atoms, for example lack a hydrogen atom as compared to the same compound according to the invention when not comprised in an inhibitor conjugate. This is sometimes referred to as the residue of the linker-drug compound in that the conjugation reaction has resulted in the removal of an atom or group as is understood in the art. This can be for example because the linker-drug compound according to the invention is conjugated to a polypeptide via for example esterification to a hydroxyl moiety.

Preferably, a polypeptide as used herein is an antibody or an antigen-binding fragment thereof. Therefore, the invention preferably relates to an antibody-drug conjugate (ADC) comprising a linker-drug compound according to the invention.

In one embodiment, the present invention relates to an ADC of formula (III)

Ab-(L-D)_{y} (III),
wherein Ab is an antibody or an antigen-binding fragment thereof,
L-D is a linker-drug compound according to the invention, i.e., L-D represents the residue of a conjugated linker-drug compound according to the invention; and
y represents an average drug-to-antibody ratio (DAR) of from 1 to 16, preferably of from 1 to 10.

As is well-known in the art, the DAR and drug load distribution can be determined, for example, by using hydrophobic interaction chromatography (HIC) or reversed phase high-performance liquid chromatography (RP-HPLC). HIC is particularly suitable for determining the average DAR.

In a preferred embodiment, the present invention relates to an ADC of formula (III), wherein the linker-drug compound according to the invention is conjugated to the antibody or antigen-binding fragment thereof through a cysteine residue of the antibody or the antigen-binding fragment.

In a more preferred embodiment, the present invention relates to an ADC of formula,

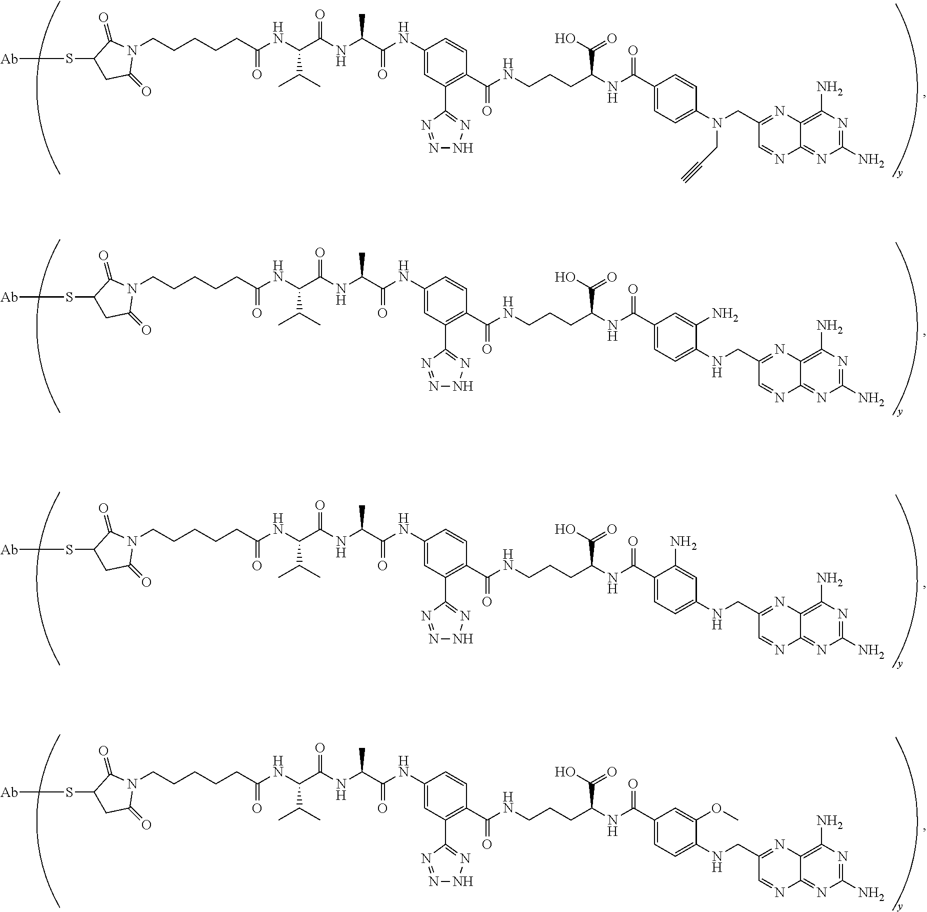

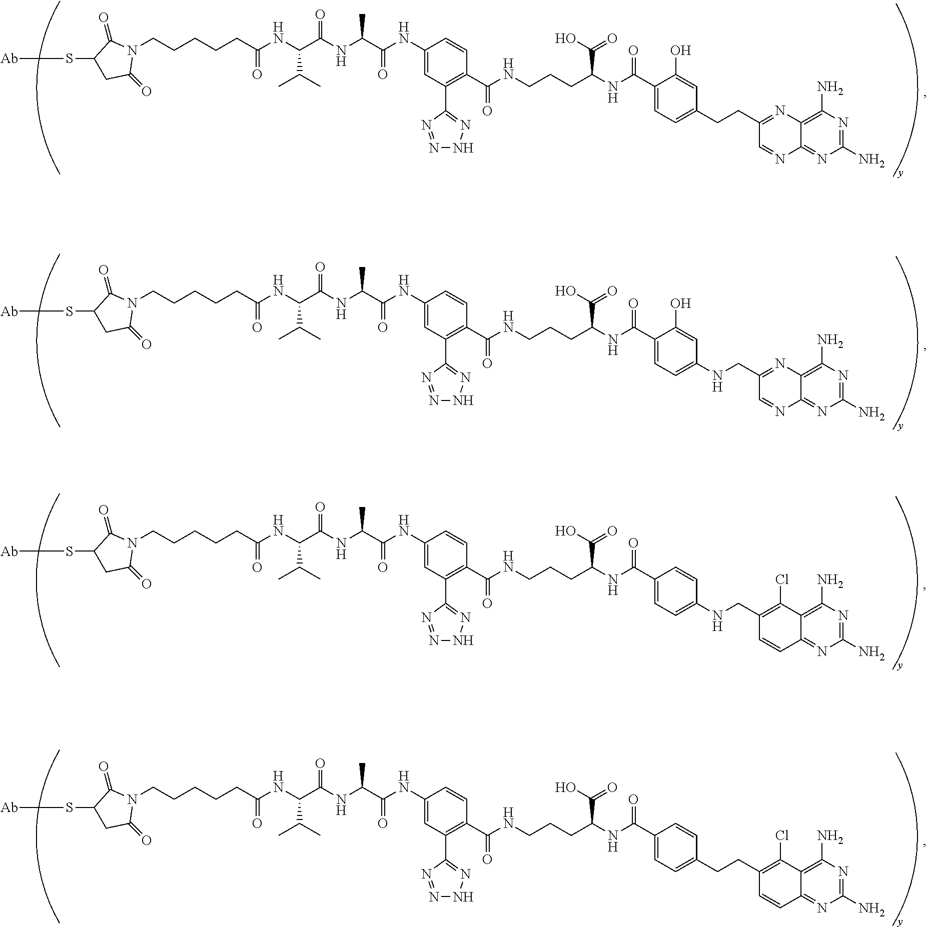

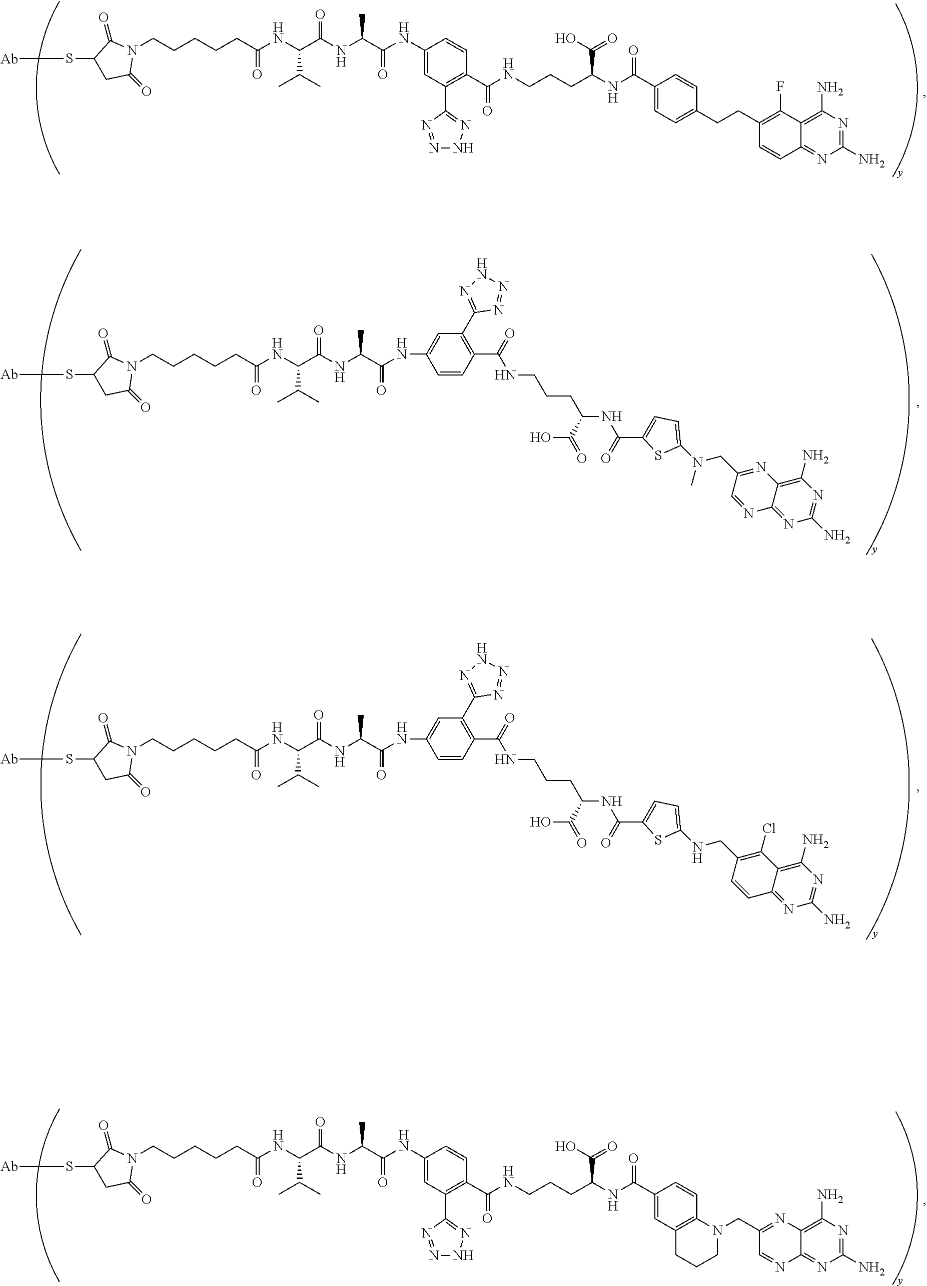

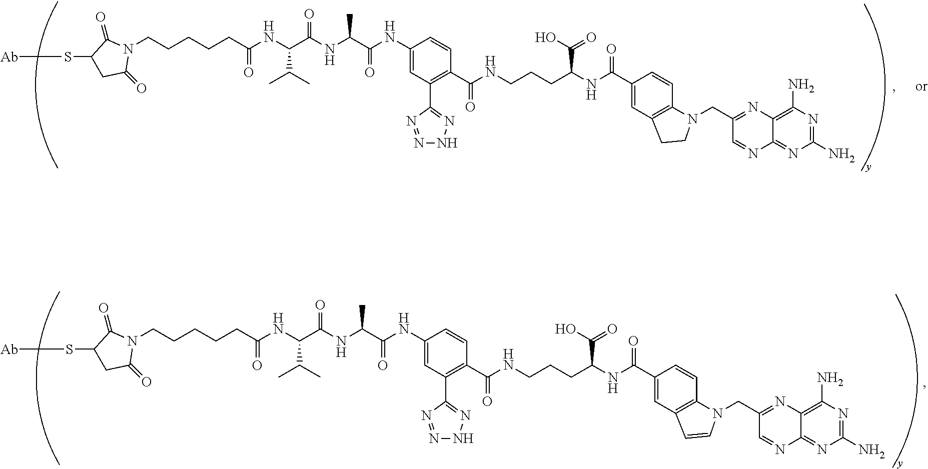

wherein
Ab is an antibody or antigen-binding fragment thereof; and
y represents an average DAR of from 1 to 16, preferably of from 1 to 10.

In an even more preferred embodiment, the present invention relates to an ADC of formula,

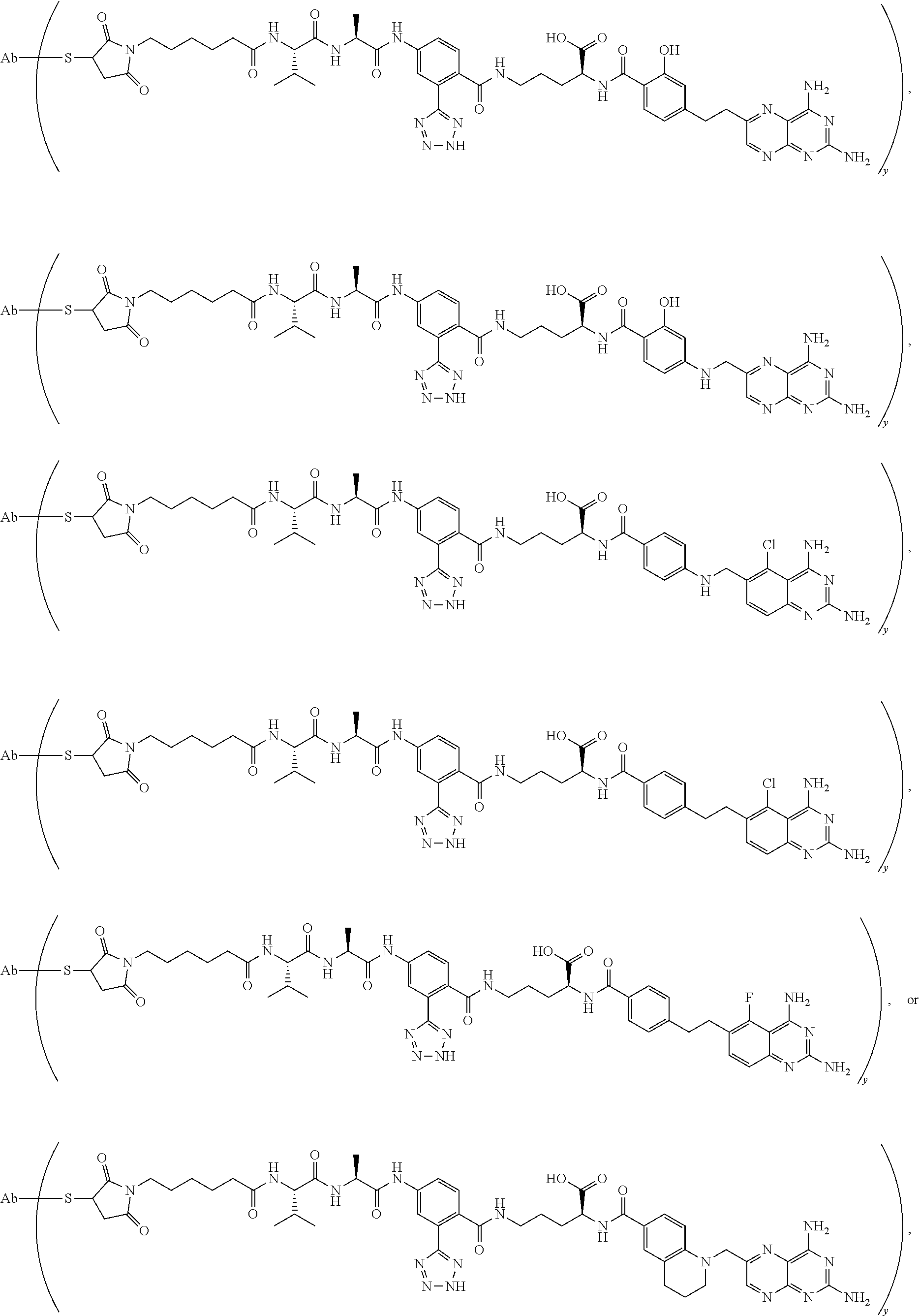

wherein
Ab is an antibody or antigen-binding fragment thereof; and
y represents an average DAR of from 1 to 16, preferably of from 1 to 10.

In a most preferred embodiment, the present invention relates to an ADC of formula

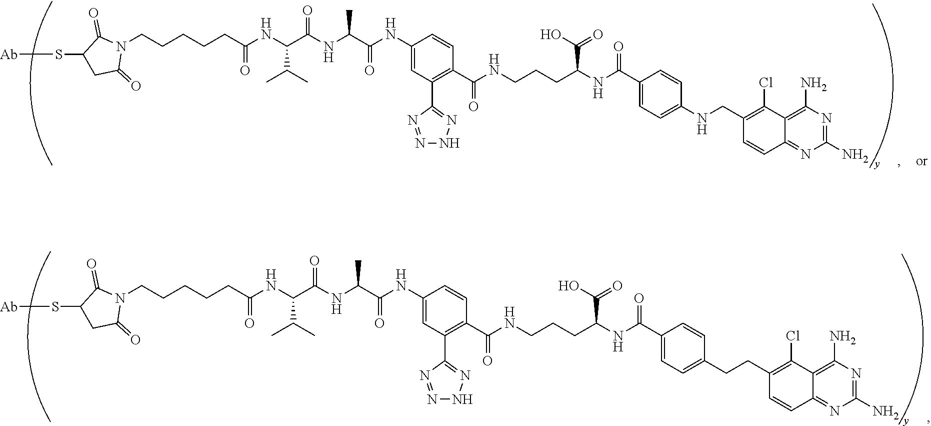

wherein
Ab is an antibody or antigen-binding fragment thereof; and
y represents an average DAR of from 1 to 16, preferably of from 1 to 10.

In the context of the present invention, Ab in the ADC formulae above can be any antibody or antigen-binding fragment thereof, preferably a monoclonal antibody (mAb) or an antigen-binding fragment thereof.

The term “antibody” as used herein preferably refers to an antibody comprising two heavy chains and two light chains. Generally, the antibody or any antigen-binding fragment thereof is one that has a therapeutic activity, but such independent efficacy is not necessarily required, as is known in the art of ADCs. The antibodies to be used in accordance with the invention may be of any isotype such as IgA, IgE, IgG, or IgM antibodies. Preferably, the antibody is an IgG antibody, more preferably an IgG₁ or IgG₂ antibody. The antibodies may be chimeric, humanized or human. Preferably, the antibodies are humanized or human. Even more preferably, the antibody is a humanized or human IgG antibody, more preferably a humanized or human IgG₁ mAb. The antibody may have κ (kappa) or λ (lambda) light chains, preferably κ (kappa) light chains, i.e., a humanized or human IgG₁-κantibody.

The term “antigen-binding fragment” as used herein includes a Fab, Fab′, F(ab′)₂, Fv, scFv or reduced IgG (rIgG) fragment, a single chain (sc) antibody, a single domain (sd) antibody, a diabody, or a minibody.

“Humanized” forms of non-human (e.g., rodent) antibodies are antibodies (e.g., non-human-human chimeric antibodies) that contain minimal sequences derived from the non-human antibody. Various methods for humanizing non-human antibodies are known in the art. For example, the antigen-binding complementarity determining regions (CDRs) in the variable regions (VRs) of the heavy chain (HC) and light chain (LC) are derived from antibodies from a non-human species, commonly mouse, rat or rabbit. These non-human CDRs may be combined with human framework regions (FRs, i.e., FR1, FR2, FR3 and FR4) of the variable regions of the HC and LC, in such a way that the functional properties of the antibodies, such as binding affinity and specificity, are at least partially retained. Selected amino acids in the human FRs may be exchanged for the corresponding original non-human species amino acids to further refine antibody performance, such as to improve binding affinity, while retaining low immunogenicity. The thus humanized variable regions are typically combined with human constant regions. Exemplary methods for humanization of non-human antibodies are the method of Winter and co-workers (Jones et al, Nature 1986, 321, 522-525; Riechmann et al, Nature 1988, 332, 323-327; Verhoeyen et al, Science 1988, 239, 1534-1536). Alternatively, non-human antibodies can be humanized by modifying their amino acid sequence to increase similarity to antibody variants produced naturally in humans. For example, selected amino acids of the original non-human species FRs are exchanged for their corresponding human amino acids to reduce immunogenicity, while retaining the antibody's binding affinity. For further details, see Jones et al, Nature 1986, 321, 522-525; Riechmann et al, Nature 1988, 332, 323-327; and Presta, Curr. Op. Struct. Biol. 1992, 2, 593-596. See also the following review articles and references cited therein: Vaswani and Hamilton, Ann. Allergy, Asthma and Immunol. 1998, 1, 105-115; Harris, Biochem. Soc. Transactions 1995, 23, 1035-1038; and Hurle and Gross, Curr. Op. Biotech. 1994, 5, 428-433.

The CDRs may be determined using the approach of Kabat (in Kabat, E. A. et al, Sequences of Proteins of Immunological Interest, 5th Ed. Public Health Service, National Institutes of Health, Bethesda, MD, NIH publication no. 91-3242, pp. 662, 680, 689 (1991)), Chothia (Chothia et al, Nature 1989, 342, 877-883) or IMGT (Lefranc, The Immunologist 1999, 7, 132-136).

Typically, the antibody is a monospecific (i.e., specific for one antigen; such antigen may be common between species or have similar amino acid sequences between species) or bispecific (i.e., specific for two different antigens of a species) antibody comprising at least one HC and LC variable region binding to an antigen target, preferably a membrane bound antigen target which may be internalizing or not internalizing, preferably internalizing.

In one particular embodiment, Ab is an anti-tumor antibody or antigen binding fragment thereof.

In another particular embodiment, the antigen target is selected from the group consisting of: annexin Al, B7H3, B7H4, BCMA, CA6, CA9, CA15-3, CA19-9, CA27-29, CA125, CA242 (cancer antigen 242), CAIX, CCR2, CCR5, CD2, CD19, CD20, CD22, CD24, CD30 (tumor necrosis factor 8), CD33, CD37, CD38 (cyclic ADP ribose hydrolase), CD40, CD44, CD47 (integrin associated protein), CD56 (neural cell adhesion molecule), CD70, CD71, CD73, CD74, CD79, CD115 (colony stimulating factor 1 receptor), CD123 (interleukin-3 receptor), CD138 (Syndecan 1), CD203c (ENPP3), CD303, CD333, CDCP1, CEA, CEACAM, Claudin 4, Claudin 7, CLCA-1 (C-type lectin-like molecule-1), CLL 1, c-MET (hepatocyte growth factor receptor), Cripto, DLL3, EGFL, EGFR, EPCAM, EphA2, EPhB3, ETBR (endothelin type B receptor), FAP, FcRL5 (Fc receptor-like protein 5, CD307), FGFR3, FOLR1 (folate receptor alpha), FRbeta, GCC (guanylyl cyclase C), GD2, GITR, GLOBO H, GPA33, GPC3, GPNMB, HER2, p95HER2, HER3, HMW-MAA (high molecular weight melanoma-associated antigen), integrin α (e.g., αvβ3 and αvβ5), IGF1R, TM4SF1 (L6), Lewis A like carbohydrate, Lewis X, Lewis Y (CD174), LGR5, LIV1, mesothelin (MSLN), MN (CA9), MUC1, MUC16, NaPi2b, Nectin-4, Notch3, PD-1, PD-L1, PSMA, PTK7, SLC44A4, STEAP-1, 5T4 (or TPBG, trophoblast glycoprotein), TF (tissue factor, thromboplastin, CD142), TF-Ag, Tag72, TNFalpha, TNFR, TROP2 (tumor-associated calcium signal transducer 2), uPAR, VEGFR and VLA.

Examples of suitable antibodies include blinatumomab (CD19), epratuzumab (CD22), iratumumab and brentuximab (CD30), vadastuximab (CD33), tetulumab (CD37), isatuximab (CD38), bivatuzumab (CD44), lorvotuzumab (CD56), vorsetuzumab (CD70), milatuzumab (CD74), polatuzumab (CD79), rovalpituzumab (DLL3), cetuximab and futuximab (EGFR), oportuzumab (EPCAM), farletuzumab (FOLR1), glembatumumab (GPNMB), trastuzumab, pertuzumab and margetuximab (HER2), etaracizumab (integrin), anetumab (mesothelin), pankomab (MUC1), enfortumab (Nectin-4), sacituzumab (TROP2), and H8, A1, and A3 (5T4).

The antibody or antigen-binding fragment thereof, if applicable, may comprise (1) a constant region that is engineered, i.e., one or more mutations may have been introduced to e.g., increase half-life, provide a site of attachment for the linker-drug and/or increase or decrease effector function; or (2) a variable region that is engineered, i.e., one or more mutations may have been introduced to e.g., provide a site of attachment for the linker-drug. Antibodies or antigen-binding fragments thereof may be produced recombinantly, synthetically, or by other known suitable methods.

ADCs according to the present invention may be wild-type or site-specific or a combination thereof, and can be produced by any method known in the art as exemplified below.

### Processes for Preparing an ADC According to the Invention

Wild-type ADCs may be produced by conjugating a linker-drug to the antibody or antigen-binding fragment thereof through e.g., the lysine ç-amino groups of the antibody, preferably using a linker-drug comprising an amine-reactive group such as an activated ester; contacting of the activated ester with the antibody or antigen-binding fragment thereof will yield the ADC. Alternatively, wild-type ADCs can be produced by conjugating the linker-drug through the free thiols of the side chains of cysteines generated through reduction of interchain disulfide bonds, using methods and conditions known in the art, see e.g., Doronina et al, Bioconjugate Chem. 2006, 17, 114-124. The manufacturing process involves partial reduction of the solvent-exposed interchain disulfides followed by modification of the resulting thiols with Michael acceptor-containing linker-drugs such as maleimide-containing linker-drugs, alfa-haloacetic amides or esters. The cysteine attachment strategy results in maximally two linker-drugs per reduced disulfide. Most human IgG molecules have four solvent-exposed disulfide bonds, and so a range of integers of from zero to eight linker-drugs per antibody is possible. The exact number of linker-drugs per antibody is determined by the extent of disulfide reduction and the number of molar equivalents of linker-drug used in the ensuing conjugation reaction. Full reduction of all four disulfide bonds gives a homogeneous construct with eight linker-drugs per antibody, while a partial reduction typically results in a heterogeneous mixture with zero, two, four, six, or eight linker-drugs per antibody.

Site-specific ADCs are preferably produced by conjugating the linker-drug to the antibody or antigen-binding fragment thereof through the side chains of engineered cysteine residues in suitable positions of the mutated antibody or antigen-binding fragment thereof. Engineered cysteines are usually capped by other thiols, such as cysteine or glutathione, to form disulfides. These capped residues need to be uncapped before linker-drug attachment can occur. Linker-drug attachment to the engineered residues is either achieved (1) by reducing both the native interchain and mutant disulfides, then re-oxidizing the native interchain cysteines using a mild oxidant such as CuSO₄ or dehydroascorbic acid, followed by standard conjugation of the uncapped engineered cysteine with a linker-drug, or (2) by using mild reducing agents which reduce mutant disulfides at a higher rate than the interchain disulfide bonds, followed by standard conjugation of the uncapped engineered cysteine with a linker-drug. Under optimal conditions, two linker-drugs per antibody or antigen-binding fragment thereof (i.e., DAR is 2) will be attached (if one cysteine is engineered into the HC or LC of the antibody or fragment). Suitable methods for site-specifically conjugating linker-drugs can for example be found in WO 2015/177360 which describes the process of reduction and re-oxidation, WO 2017/137628 which describes a method using mild reducing agents and WO 2018/215427 which describes a method for conjugating both the reduced interchain cysteines and the uncapped engineered cysteines.

### Pharmaceutical Compositions

In a further aspect, the invention provides a composition comprising a linker-drug compound or an inhibitor conjugate according to the invention, preferably wherein the composition is a pharmaceutical composition, more preferably further comprising a pharmaceutically acceptable excipient. Such composition is referred to hereinafter as a composition according to the invention. The composition may for example be a liquid formulation, a lyophilized formulation, or in the form of e.g., capsules or tablets. Typically, pharmaceutical formulations comprising small molecules in the form of capsules or tablets, such as linker-drug compounds according to the invention, comprise a diluent. Suitable water soluble diluents include sugars, sugar alcohols, polysaccharides and cyclodextrins. Suitable non-water soluble diluents include calcium phosphate, calcium sulphate, starches, modified starches and microcrystalline cellulose. Additionally, pharmaceutical formulations comprising small molecules, such as linker-drug compounds according to the invention, may comprise a binder. Suitable binders include gelatin, cellulose derivatives, polymers such as crosslinked polyvinylpyrrolidone (crospovidone) or copolyvidone, and polyethylene glycol. Pharmaceutical formulations comprising small molecules, such as linker-drug compounds according to the invention, may further comprise a disintegrant. Suitable disintegrants include crosslinked polymers, such as crospovidone and crosslinked carboxymethyl cellulose sodium (croscarmellose sodium) and sodium starch glycolate. Additionally, pharmaceutical formulations comprising small molecules, such as linker-drug compounds according to the invention, may comprise glidants such as fumed silica, talc, and magnesium carbonate; lubricants such as talc or silica, vegetable stearin, magnesium stearate or stearic acid; preservatives such as antioxidants or parabens; colourants; sweeteners and/or flavours.

Typically, pharmaceutical compositions comprising inhibitor conjugates, including ADCs, or linker-drug compounds according to the invention take the form of lyophilized cakes (lyophilized powders), which require (aqueous) dissolution (i.e., reconstitution) before intravenous infusion, or frozen (aqueous) solutions, which require thawing before use. Accordingly, in preferred embodiments, the invention provides a lyophilized composition comprising a linker-drug compound or an inhibitor conjugate according to the invention, preferably wherein the composition is a pharmaceutical composition, more preferably further comprising a pharmaceutically acceptable excipient. In further preferred embodiments, the invention provides a frozen composition comprising water and a linker-drug compound or an inhibitor conjugate according to the invention, preferably wherein the composition is a pharmaceutical composition, more preferably further comprising a pharmaceutically acceptable excipient. In this context, the frozen solution is preferably at atmospheric pressure, and the frozen solution was preferably obtained by freezing a liquid composition according to the invention at temperatures below 0° C. Suitable pharmaceutically acceptable excipients for inclusion into the pharmaceutical composition (before freeze-drying) in accordance with the present invention include buffer solutions (e.g., citrate, amino acids such as histidine, or succinate containing salts in water), lyoprotectants (e.g., sucrose, trehalose), tonicity modifiers (e.g., chloride salts such as sodium chloride), surfactants (e.g., polysorbate), and bulking agents (e.g., mannitol, glycine). Excipients used for freeze-dried protein formulations are selected for their ability to prevent protein denaturation during the freeze-drying process as well as during storage.

### Medical Uses

In a further aspect, the invention provides a linker-drug compound, an inhibitor conjugate (preferably an antibody-drug conjugate), or a composition according to the invention, for use as a medicament, preferably for the treatment of cancer, autoimmune or infectious diseases. These linker-drug compounds, conjugates, and compositions are collectively referred to hereinafter as products for use according to the invention.

In one embodiment, the products for use according to the invention are for use in the treatment of a solid tumor or hematological malignancy.

In a second embodiment, the products for use according to the invention are for use in the treatment of an autoimmune disease.

In a third embodiment, the products for use according to the invention are for use in the treatment of an infectious disease, such as a bacterial, viral, parasitic, or other infection.

A cancer in the context of the present invention, preferably is a tumor expressing the antigen to which the products for use according to the invention are directed. Such tumor may be a solid tumor or hematological malignancy. Examples of tumors or hematological malignancies that may be treated with products for use according to the invention as defined above may include, but are not limited to, breast cancer; brain cancer (e.g., glioblastoma); head and neck cancer; thyroid cancer; parotic gland cancer; adrenal cancer (e.g., neuroblastoma, paraganglioma, or pheochromocytoma); bone cancer (e.g., osteosarcoma); soft tissue sarcoma (STS); ocular cancer (e.g., uveal melanoma); esophageal cancer; gastric cancer; small intestine cancer; colorectal cancer; urothelial cell cancer (e.g., bladder, penile, ureter, or renal cancer); ovarian cancer; uterine cancer; vaginal, vulvar and cervical cancer; lung cancer (especially non-small cell lung cancer (NSCLC) and small-cell lung cancer (SCLC)); melanoma; mesothelioma (especially malignant pleural and abdominal mesothelioma); liver cancer (e.g., hepatocellular carcinoma); pancreatic cancer; skin cancer (e.g., basalioma, squamous cell carcinoma, or dermatofibrosarcoma protuberans); testicular cancer; prostate cancer; acute myeloid leukemia (AML); chronic myeloid leukemia (CML); chronic lymphatic leukemia (CLL); acute lymphoblastic leukemia (ALL); myelodysplastic syndrome (MDS); blastic plasmacytoid dendritic cell neoplasia (BPDCN); Hodgkin's lymphoma; non-Hodgkin's lymphoma (NHL) (including follicular lymphoma (FL), CNS lymphoma, and diffuse large B-cell lymphoma (DLBCL)); light chain amyloidosis; plasma cell leukemia; and multiple myeloma (MM).

One aspect of the invention relates to a method of treating cancer, especially a solid tumor, which comprises administering an effective amount of the antibody-drug conjugate according to the invention to a patient in need thereof. The Ab is an anti-tumor antibody or antigen-binding fragement thereof. The patient can be human, mammal, or other animal. The effective amount for treatment can be determined based on known use of antibody-drug conjugates and antifolates in treating cancer as well as in light of the data in the present examples.

An autoimmune disease in the context of the present invention, preferably is an autoimmune disease associated with the antigen to which the products for use according to the invention are directed. An autoimmune disease represents a condition arising from an abnormal immune response to normal body cells and tissues. There is a wide variety of at least 80 types of autoimmune diseases. Some diseases are organ specific and are restricted to affecting certain tissues, while others resemble systemic inflammatory diseases that impact many tissues throughout the body. The appearance and severity of these signs and symptoms depend on the location and type of inflammatory response that occurs and may fluctuate over time. Examples of autoimmune diseases that may be treated with products for use according to the invention as defined above may include, but are not limited to, rheumatoid arthritis; juvenile dermatomyositis; psoriasis; psoriatic arthritis; lupus; sarcoidosis; Crohn's disease; eczema; nephritis; uveitis; polymyositis; neuritis including Guillain-Barre syndrome; encephalitis; arachnoiditis; systemic sclerosis; autoimmune mediated musculoskeletal and connective tissue diseases; neuromuscular degenerative diseases including Alzheimer's disease, multiple sclerosis (MS), amyotrophic lateral sclerosis (ALS), neuromyelitis optica, and large, middle size, small vessel Kawasaki and Henoch Schonlein vasculitis; cold and warm agglutinin disease; autoimmune hemolytic anemia; type 1 diabetes mellitus; Hashimoto's thyroiditis; Graves' disease; Graves' ophthalmopathy; adrenalitis; hypophysitis; pemphigus vulgaris; Addison's disease; ankyloses spondylitis; Behcet's syndrome; celiac disease; Goodpasture's syndrome; myasthenia gravis; sarcoidosis; scleroderma; primary sclerosing cholangitis, epidermolysis bullosa acquisita, and bullous pemphigoid.

Preferably, the autoimmune disease which is treated in the context of the present invention is rheumatoid arthritis.

An infectious disease in the context of the present invention, preferably is an infectious disease associated with the antigen to which the products for use according to the invention are directed. Such infectious disease may be a bacterial, viral, parasitic or other infection. Examples of infectious diseases that may be treated with products for use according to the invention as defined above may include, but are not limited to, malaria; toxoplasmosis; pneumocystis jirovecii melioidosis; shigellosis; listeria; cyclospora; mycobacterium leprae; tuberculosis; and infectious prophylaxis in immune compromised individuals, such as in HIV-positive individuals, individuals on immunosuppressive treatment, or individuals with inborn errors such as cystic fibrosis or benign proliferative diseases (e.g., mola hydatidosa or endometriosis).

Products for use according to the invention as described herein can be for the use in the manufacture of a medicament as described herein. Products for use according to the invention as described herein are preferably for methods of treatment wherein the products for use are administered to a subject, preferably to a subject in need thereof, in a therapeutically effective amount. Thus, alternatively, or in combination with any of the other embodiments, in an embodiment, the present invention relates to a use of products for use according to the invention for the manufacture of a medicament for the treatment of cancer, autoimmune or infectious diseases, in particular for the treatment of cancer. For illustrative, non-limitative, cancers or other diseases to be treated according to the invention: see hereinabove.

Alternatively, or in combination with any of the other embodiments, in an embodiment, the present invention relates to a method for treating cancer, autoimmune or infectious diseases, in particular cancer, which method comprises administering to a subject in need of said treatment a therapeutically effective amount of a product for use according to the invention. For illustrative, non-limitative, cancers or other diseases to be treated according to the invention: see hereinabove.

Products for use according to the invention are for administration to a subject. Products for use according to the invention can be used in the methods of treatment described hereinabove by administration of an effective amount of the composition to a subject in need thereof. The term “subject” as used herein refers to all animals classified as mammals and includes, but is not restricted to, primates and humans. The subject is preferably a human. The expression “therapeutically effective amount” means an amount sufficient to effect a desired response, or to ameliorate a symptom or sign. A therapeutically effective amount for a particular subject may vary depending on factors such as the condition being treated, the overall health of the subject, the method, route, and dose of administration and the severity of side effects.

In further embodiments, the invention provides the product for use according to the invention, wherein the use is combined with one or more other therapeutic agents. Products for use according to the invention may be used concomitantly or sequentially with the one or more other therapeutic agents.

Suitable chemotherapeutic agents include alkylating agents, such as nitrogen mustards, hydroxyurea, nitrosoureas, tetrazines (e.g., temozolomide) and aziridines (e.g., mitomycin); drugs interfering with the DNA damage response, such as PARP inhibitors, ATR and ATM inhibitors, CHK1 and CHK2 inhibitors, DNA-PK inhibitors, and WEE1 inhibitors; anti-metabolites, such as antifolates (e.g., pemetrexed), fluoropyrimidines (e.g, gemcitabine), deoxynucleoside analogues and thiopurines; anti-microtubule agents, such as vinca alkaloids and taxanes; topoisomerase I and II inhibitors; cytotoxic antibiotics, such as anthracyclines and bleomycins; hypomethylating agents such as decitabine and azacitidine; histone deacetylase inhibitors; all-trans retinoic acid; and arsenic trioxide. Suitable radiation therapeutics include radio-isotopes, such as ¹³¹I-metaiodobenzylguanidine (MIBG), ³²P as sodium phosphate, ²²³R^{a} chloride, ⁸⁹Sr chloride and ¹⁵³Sm diamine tetramethylene phosphonate (EDTMP). Suitable agents to be used as hormonal therapeutics include inhibitors of hormone synthesis, such as aromatase inhibitors and GnRH analogues; hormone receptor antagonists, such as selective estrogen receptor modulators (e.g., tamoxifen and fulvestrant) and antiandrogens, such as bicalutamide, enzalutamide and flutamide; CYP17A1 inhibitors, such as abiraterone; and somatostatin analogs.

Targeted therapeutics are therapeutics that interfere with specific proteins involved in tumorigenesis and proliferation and may be small molecule drugs; proteins, such as therapeutic antibodies; peptides and peptide derivatives; or protein-small molecule hybrids, such as ADCs. Examples of targeted small molecule drugs include mTor inhibitors, such as everolimus, temsirolimus and rapamycin; kinase inhibitors, such as imatinib, dasatinib and nilotinib; VEGF inhibitors, such as sorafenib and regorafenib; EGFR/HER2 inhibitors, such as gefitinib, lapatinib, and erlotinib; and CDK4/6 inhibitors, such as palbociclib, ribociclib and abemaciclib. Examples of peptide or peptide derivative targeted therapeutics include proteasome inhibitors, such as bortezomib and carfilzomib.

Suitable anti-inflammatory drugs include D-penicillamine, azathioprine and 6-mercaptopurine, cyclosporine, anti-TNF biologicals (e.g., infliximab, etanercept, adalimumab, golimumab, certolizumab, or certolizumab pegol), lenflunomide, abatacept, tocilizumab, anakinra, ustekinumab, rituximab, daratumumab, ofatumumab, obinutuzumab, secukinumab, apremilast, acetretin, and JAK inhibitors (e.g., tofacitinib, baricitinib, or upadacitinib).

Immunotherapeutic agents include agents that induce, enhance or suppress an immune response, such as cytokines (IL-2 and IFN-α); immuno modulatory imide drugs, e.g., thalidomide, lenalidomide, pomalidomide, or imiquimod; therapeutic cancer vaccines, e.g., talimogene laherparepvec; cell based immunotherapeutic agents, e.g., dendritic cell vaccines, adoptive T-cells, or chimeric antigen receptor-modified T-cells; and therapeutic antibodies that can trigger antibody-dependent cell-mediated cytotoxicity (ADCC), antibody-dependent cellular phagocytosis (ADCP) or complement-dependent cytotoxicity (CDC) via their Fc region when binding to membrane bound ligands on a cell.

In the context of the invention, treatment is preferably preventing, reverting, curing, ameliorating, and/or delaying the cancer, autoimmune or infectious disease. This may mean that the severity of at least one symptom of the cancer, autoimmune or infectious disease has been reduced, and/or at least a parameter associated with the cancer, autoimmune or infectious disease has been improved. Preferably, such parameter is associated with folate activity.

In the context of the invention, a subject may survive and/or may be considered as being disease free. Alternatively, the disease or condition may have been stopped or delayed. In the context of the invention, an improvement of quality of life and observed pain relief may mean that a subject may need less pain relief drugs than at the onset of the treatment. “Less” in this context may mean 5% less, 10% less, 20% less, 30% less, 40% less, 50% less, 60% less, 70% less, 80% less, 90% less. A subject may no longer need any pain relief drug. This improvement of quality of life and observed pain relief may be seen, detected or assessed after at least one week, two weeks, three weeks, four weeks, one month, two months, three months, four months, five months, six months or more of treatment in a subject and compared to the quality of life and observed pain relief at the onset of the treatment of said subject.

### General Definitions

Linker-drug compounds according to the invention may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof. Accordingly, the chemical structures depicted herein encompass all possible tautomeric forms of the illustrated or identified compounds. It is also understood that some isomeric forms such as diastereomers, enantiomers and geometrical isomers can be separated by physical and/or chemical methods by those skilled in the art. When a structural formula or chemical name is understood by the skilled person to have chiral centers, yet no chirality is indicated, for each chiral center individual reference is made to all three of either the racemic mixture, the pure R enantiomer, and the pure S enantiomer. When the structure of a compound is depicted as a specific enantiomer, it is to be understood that the invention of the present application is not limited to that specific enantiomer. When two moieties are said to together form a bond, this implies the absence of these moieties as atoms, and compliance of valence being fulfilled by a replacing electron bond. All this is known in the art.

The compounds disclosed in this description and in the claims may further exist as exo and endo regioisomers. Unless stated otherwise, the description of any compound in the description and in the claims is meant to include both the individual exo and the individual endo regioisomer of a compound, as well as mixtures thereof. Furthermore, the compounds disclosed in this description and in the claims may exist as cis and trans isomers. Unless stated otherwise, the description of any compound in the description and in the claims is meant to include both the individual cis and the individual trans isomer of a compound, as well as mixtures thereof. As an example, when the structure of a compound is depicted as a cis isomer, it is to be understood that the corresponding trans isomer or mixtures of the cis and trans isomer are not excluded from the invention of the present application.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g., about 10) preferably means that the value may be the given value more or less 1% of the value.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature (RT) to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such charge more often than that it does not bear or carry such charge. As such, an atom that is indicated in this disclosure to be charged could be non-charged under specific conditions, and a neutral moiety could be charged under specific conditions, as is understood by a person skilled in the art.

All patent and literature references cited in the present specification are hereby incorporated by reference in their entirety.

The following Examples are offered for illustrative purposes only, and are not intended to limit the scope of the present invention in any way.

### EXAMPLES

### General

All solvents used were reagent grade or HPLC grade from various vendors. NMR spectra were recorded on a Bruker AVANCE400 (400 MHz for ¹H; 100 MHz for ¹³C). Chemical shifts are reported in ppm relative to tetramethylsilane as an internal standard, or residual undeuterated solvent. Mass spectra were recorded using a Waters UPLC-MS (ESI, equipped with a SQ-detector 3100 or a SQ-detector 2) with a reversed Phase C18 bridged ethylsiloxane-silica hybrid column (Waters ACQUITY UPLC® BEH C18 1.7 μm particle size, 2.1×50 mm) at a flow rate of 0.4 mL/min (acetonitrile (MeCN)/water×0.1% formic acid (FA)). Purifications by preparative HPLC were performed using a Shimadzu Prominence 20AP system equipped with a Waters SunFire Prep C18 OBD 5 μm column (19×150 mm) at a flow rate of 17 mL/min (MeCN/water×0.1% trifluoroacetic acid (TFA)). Microwave reactions were performed in a Biotage Initiator+ instrument.

### Hydrophobic Interaction Chromatography (HIC) for Characterization of ADCs

For analytical HIC, 5-10 μL of sample (1 mg/mL) was injected onto a TSKgel Butyl-NPR column (4.6 mm ID×3.5 cm L, Tosoh Bioscience, Cat. no. 14947). The elution method consisted of a linear gradient from 100% Buffer A (25 mM sodium phosphate, 1.5 M ammonium sulphate, pH 6.95) to 100% of Buffer B (25 mM sodium phosphate, pH 6.95, 20% isopropanol) at 0.4 mL/min over 20 min. A Waters Acquity H-Class UPLC system equipped with PDA-detector and Empower software was used. Absorbance was measured at 214 nm and the retention time of ADCs was determined.

### Size Exclusion Chromatography (SEC) for Characterization of ADCs

For analytical SEC, 5 μL of sample (1 mg/mL) was injected onto a TSKgel G3000SWXL column (5 μm, 7.8 mm ID×30 cm L, Tosoh Bioscience, Cat. no. 08541) equipped with a TSKgel SWXL Guard column (7 μm, 6.0 mm ID×4.0 cm L, Tosoh Bioscience, Cat. no. 08543). The elution method consisted of elution with 100% 50 mM sodium phosphate, 300 mM NaCl, pH 7.5 at 0.6 mL/min for 30 min. The column temperature was maintained at 25° C. A Waters Acquity H-Class UPLC system equipped with PDA-detector and Empower software was used. Absorbance was measured at 214 nm to quantify the amount of HMW species.

### General Procedure XXA: HATU-Assisted Amide Coupling

The carboxylic acid (1.0 eq) and amine (1.0-1.5 eq) were dissolved in dimethylformamide (DMF; 0.175 M). 1-[bis(dimethylamino)methylene]-1H-1,2,3-triazolo[4,5-b]pyridinium 3-oxide hexafluorophosphate (hexafluorophosphate azabenzotriazole tetramethyl uronium, HATU; 1.2 eq) and N,N-diisopropylethylamine (DIPEA; 6.0 eq) were added at RT and the mixture was stirred for 30 min. After concentration, the product was recrystallized from methanol (MeOH) or purified by flash chromatography, as indicated.

### General Procedure XXB: Reduction of Phenyl Azides with Pd/C and Hydrogen

To a solution of azide (1.0 eq) in DMF (0.074 M) under N₂ was added 10 wt % of “palladium, 10 wt % on activated carbon”. The mixture was purged with hydrogen gas and stirred vigorously until UPLC-analysis indicated full conversion (˜45 min). The reaction was purged with N₂ and filtered over Celite®. The filtrate was concentrated in vacuo and purified as indicated.

### General Procedure XXC: Formamide Deprotection and or Ester Hydrolysis

The ester (1.0 eq) was dissolved/suspended in methanol/dimethyl sulfoxide (MeOH/DMSO; 5:1, 0.36 M) and cooled to 0° C. Aqueous NaOH (2.0 M, 3-24 eq) was added dropwise and the resulting solution was stirred at RT until complete by UPLC-analysis (1-6 h). The reaction mixture was diluted with water (˜4×), and after cooling to 0° C., the pH was adjusted to pH ˜8.5 with aq. HCl (1.0 M). If no precipitation occurred, the solution was washed with ethyl acetate (EtOAc; 3×). For suspensions this step was omitted. The water phase was further diluted with water (˜3×) and the pH was adjusted to approximately pH 4.7 with aq. acetic acid (AcOH; 1.0 M). The resulting suspension was vigorously stirred for 30 min and filtered. Occasionally, a gelatinous mixture was obtained, and gentle heating with a heat gun then afforded a suspension. The solid was washed with MeOH and ether, and dried under vacuum.

### General Procedure XXD: Deprotection HATU-Coupling

A solution of the starting material (1.0 eq) in dichloromethane (DCM)/TFA (1:1) was stirred for 30 min at 0° C., diluted with DCM and concentrated in vacuo. The residue was coevaporated twice with DCM and washed with ether. The obtained solid TFA salt was taken up in DMF (˜0.1 M) and cooled to 0° C. with an ice bath, carboxylic acid (1 eq) was added followed by HATU (1.2 eq) and DIPEA (6 eq). The resulting mixture was stirred for 15 to 120 min at 0° C. and was concentrated in vacuo. The crude product was suspended in MeCN (˜0.04 M), stirred for 30 min and was subsequently filtered, followed by drying on air.

### General Procedure XXE: Saponification

To a cooled suspension of the ester (1.0 eq) in tetrahydrofuran (THF; ˜0.05 M) was added a solution of LiOH (5 eq) in water and the resulting mixture was stirred for 30 to 120 min at 0° C. The obtained solution was kept at 0° C. and AcOH (10 eq) was added and stirred for another 30 min. Next, the mixture was diluted with toluene and concentrated in vacuo and purified as indicated.

### Preparation of (S)-5-(4-azidobenzamido)-2-(4-(2-(2,4-diaminopteridin-6-yl)ethyl)benzamido) pentanoic acid (XX5) and (S)-5-(4-aminobenzamido)-2-(4-(2-(2,4-diaminopteridin-6-yl) ethyl)benzamido)pentanoic acid (XX7)

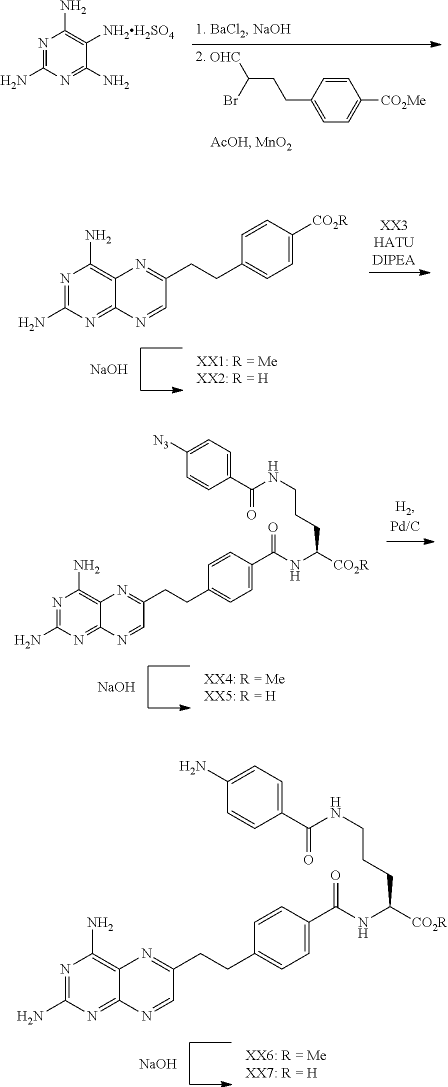

### Methyl (S)-2-amino-5-(4-azidobenzamido)pentanoate (XX3)

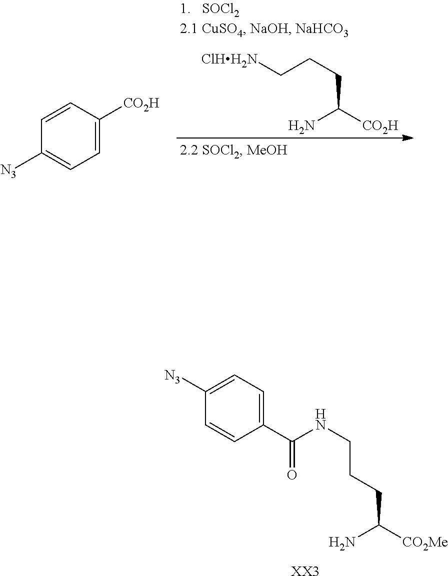

Thionyl chloride (3.76 mL, 51.5 mmol) and DMF (5 drops) were added to a suspension of 4-azidobenzoic acid (7.00 g, 42.9 mmol) in DCM (150 mL) under N₂. The reaction was heated to gentle reflux for 2 h, cooled to RT, concentrated and the solid was gently broken up into a powder. In a separate flask, a solution of L-Ornithine hydrochloride (L-Om-OH HCl) (6.70 g, 38.9 mmol) and NaOH (3.11 g, 77.8 mmol) in water (67 mL) was treated with a solution of CuSO₄·5H₂O (4.86 g, 19.5 mmol) in water (67 mL) at RT, resulting in a deep blue solution. To this solution was added NaHCO₃ (3.93 g, 46.7 mmol). Once dissolved, the dark blue solution was poured directly onto the crude, solid 4-azidobenzoyl chloride (7.78 g, 42.8 mmol) at RT, and the mixture was stirred vigorously at RT for 16 h. The suspension was filtered and the solid was washed with water (2×20 mL), ethanol (2×20 mL) and ether (3×40 mL), affording 4.97 g of a solid. The material was taken up in MeOH (140 mL) and thionyl chloride (13.0 mL) was added at 0° C. over 45 min under N₂. The reaction was then stirred at RT for 18 h and was then concentrated. Subsequently, a portion of the material was purified by solid phase extraction (SPE; Biotage, SCX-2) to give XX3 (1.20 g) as a waxy colorless solid.

¹H NMR (400 MHz, DMSO-d₆) ppm=8.49 (t, J=5.5 Hz, 1H), 7.88 (d, J=8.6 Hz, 2H), 7.19 (d, J=8.6 Hz, 2H), 3.61 (s, 3H), 3.37-3.31 (m, 1H), 3.24 (q, J=6.1 Hz, 2H), 2.13 (br s, 2H), 1.64-1.43 (m, 4H).

MS (ESI⁺) calc. for C₁₃H₁₈N₅O₃⁺ [M+H]⁺ 292.14, found 292.20.

### Methyl 4-(2-(2,4-diaminopteridin-6-yl)ethyl)benzoate (XX1)

A mixture of pyrimidine-2,4,5,6-tetraamine sulfate (6.00 g, 25.2 mmol), barium chloride dihydrate (6.16 g, 25.2 mmol), and water (145 mL) was stirred at RT for 90 min. The mixture was warmed to 70° C., filtered, and the filtrate was cooled to RT and the pH adjusted to pH 3.5 with 10% NaOH (aq). A portion of this solution (116 mL) was mixed with crude methyl 4-(3-bromo-4-oxobutyl)benzoate (6.06 g, 21.2 mmol) (prepared as described in: Chen et al, J. Heterocycl. Chem. 2015, 52, 1565-1569) in MeCN (43.8 mL), and AcOH (34.7 mL) was then added under stirring using a RT water bath. After 5 min, MnO₂ (11.6 g) was added and the mixture was stirred at RT for 1 h and 45 min. The reaction was filtered over Celite® and the residue was washed with MeCN/water (7:3). The filtrate was concentrated and washed with water (125 mL). Residual water was azeotropically removed with MeCN. The residue was then triturated twice with hot MeCN (125 mL) and the solid was then washed with ether and dried under vacuum to give XX1(1.31 g, 20%) as a brown solid.

¹H NMR (400 MHz, DMSO-d₆) ppm=8.65 (s, 1H), 8.38 (br s, 1H), 8.31 (br s, 1H), 7.87 (d, J=8.0 Hz, 2H), 7.40 (d, J=8.0 Hz, 2H), 7.27 (br s, 2H), 3.82 (s, 3H), 3.19 (s, 4H).

MS (ESI⁺) calc. for C₁₆H₁₇N₆O₂⁺ [M+H]⁺ 325.14, found 325.12.

### 4-(2-(2,4-Diaminopteridin-6-yl)ethyl)benzoic acid (XX2)

Ester (XX1) (245 mg, 0.755 mmol) was suspended in 2-methoxyethanol (6 mL) and 0.5 N NaOH (aq) (6 mL) was added at RT. The suspension was stirred for 24 h and was subsequently filtered. The residue was washed with 2-methoxyethanol/water (1:1, 1 mL) and the filtrate was then acidified to pH 4.5 with glacial AcOH (0.325 mL). Stored at 4° C. for 16 h, filtered, and the solid was washed with water (2×3 mL) and dried under vacuum to give XX2 (140 mg, 60%) as a brown solid.

¹H NMR (400 MHz, DMSO-d₆) ppm=12.78 (br s, 1H), 8.56 (s, 1H), 7.85 (d, J=8.1 Hz, 2H), 7.56 (br s, 2H), 7.37 (d, J=8.1 Hz, 2H), 6.54 (br s, 2H), 3.19-3.11 (s, 4H).

MS (ESI⁺) calc. for C₁₅H₁₅N₆O₂⁺ [M+H]⁺ 311.13, found 311.34.

### Methyl (S)-5-(4-azidobenzamido)-2-(4-(2-(2,4-diaminopteridin-6-yl)ethyl)benzamido) pentanoate (XX4)

According to general procedure XXA, acid XX2 (235 mg, 0.757 mmol) was reacted with amine XX3 (243 mg, 0.833 mmol) and purified by recrystallization from MeOH, to give ester XX4 (321 mg, 73%) as a brown solid.

¹H NMR (400 MHz, DMSO-d₆) ppm=8.66 (d, J=7.4 Hz, 1H), 8.55 (s, 1H), 8.47 (t, J=5.6 Hz, 1H), 7.90-7.86 (m, 2H), 7.79 (d, J=8.2 Hz, 2H), 7.59 (br s, 2H), 7.34 (d, J=8.2 Hz, 2H), 7.21-7.17 (m, 2H), 6.54 (br s, 2H), 4.47-4.42 (m, 1H), 3.63 (s, 3H), 3.30-3.25 (m, 2H), 3.17-3.12 (m, 4H), 1.91-1.75 (m, 2H), 1.70-1.52 (m, 2H).

MS (ESI⁺) calc. for C₂₈H₃₀N₁₁O₄⁺ [M+H]⁺ 584.25, found 584.49.

### (S)-5-(4-Azidobenzamido)-2-(4-(2-(2,4-diaminopteridin-6-yl)ethyl)benzamido) pentanoic acid (XX5)

The hydrolysis of ester XX4 (36 mg, 0.062 mmol) with NaOH (3 eq) was carried out according to general procedure XXC, to give acid XX5 (27 mg, 75%) as a yellow solid.

¹H NMR (400 MHz, DMSO-d₆) ppm=12.60 (br s, 1H), 8.56 (s, 1H), 8.52 (d, J=7.7 Hz, 1H), 8.48 (t, J=5.6 Hz, 1H), 7.88 (d, J=8.5 Hz, 2H), 7.80 (d, J=8.2 Hz, 2H), 7.65 (br s, 2H), 7.34 (d, J=8.2 Hz, 2H), 7.19 (d, J=8.5 Hz, 2H), 6.60 (s, 2H), 4.42-4.33 (m, 1H), 3.27 (q, J=6.4 Hz, 2H), 3.15 (s, 4H), 1.92-1.73 (m, 2H), 1.70-1.54 (m, 2H).

¹³C NMR (100 MHz, DMSO-d₆) ppm=173.8, 166.4, 165.2, 162.8, 162.3, 154.2, 150.4, 148.4, 144.9, 142.1, 131.7, 131.2, 129.1, 128.3, 127.5, 121.4, 118.8, 52.4, 38.9, 35.3, 33.9, 28.2, 26.2.

MS (ESI⁺) calc. for C₂₇H₂₈N₁₁O₄⁺ [M+H]⁺ 570.23, found 570.54.

### Methyl (S)-5-(4-aminobenzamido)-2-(4-(2-(2,4-diaminopteridin-6-yl)ethyl)benzamido) pentanoate (XX6)

Reduction of azide XX4 (250 mg, 0.482 mmol) was carried out according to general procedure XXB. Purification by flash chromatography (silica gel, MeOH:DCM 0:1 to 1:3) afforded aniline XX6 (151 mg, 63%) as a yellow solid.

¹H NMR (400 MHz, DMSO-d₆) ppm=8.68 (d, J=7.5 Hz, 1H), 8.55 (s, 1H), 7.98 (t, J=5.6 Hz, 1H), 7.80 (d, J=8.1 Hz, 2H), 7.63 (br s, 2H), 7.55 (d, J=8.6 Hz, 2H), 7.34 (d, J=8.1 Hz, 2H), 6.58 (br s, 2H), 6.52 (d, J=8.6 Hz, 2H), 5.59 (br s, 2H), 4.45-4.40 (m, 1H), 3.63 (s, 3H), 3.22 (q, J=6.2 Hz, 2H), 3.15 (s, 4H), 1.88-1.74 (m, 2H), 1.67-1.49 (m, 2H).

MS (ESI⁺) calc. for C₂₈H₃₂N₉O₄⁺ [M+H]⁺ 558.26, found 558.51.

### (S)-5-(4-Aminobenzamido)-2-(4-(2-(2,4-diaminopteridin-6-yl)ethyl)benzamido) pentanoic acid (XX7)

The hydrolysis of ester XX6 (151 mg, 0.271 mmol) with NaOH (3 eq) was carried out according to general procedure XXC, to give acid XX7 (112 mg, 75%) as a yellow solid.

¹H NMR (400 MHz, DMSO-d₆) ppm=12.52 (br s, 1H), 8.56 (s, 1H), 8.53 (d, J=7.7 Hz, 1H), 7.98 (t, J=5.6 Hz, 1H), 7.80 (d, J=8.2 Hz, 2H), 7.65 (br s, 2H), 7.55 (d, J=8.5 Hz, 2H), 7.34 (d, J=8.2 Hz, 2H), 6.62 (br s, 2H), 6.52 (d, J=8.5 Hz, 2H), 5.58 (br s, 2H), 4.41-4.33 (m, 1H), 3.22 (td, J=6.6, 6.0 Hz, 2H), 3.15 (s, 4H), 1.91-1.71 (m, 2H), 1.67-1.50 (m, 2H).

¹³C NMR (100 MHz, DMSO-d₆) ppm=173.8, 166.4, 166.2, 162.8, 162.3, 154.2, 151.5, 150.4, 148.4, 144.9, 131.8, 128.6, 128.3, 127.6, 121.4, 112.5, 52.6, 38.6, 35.3, 33.9, 28.2, 26.5.

MS (ESI⁺) calc. for C₂₇H₃₀N₉O₄⁺ [M+H]⁺ 544.24, found 544.52.

### Preparation of (S)-5-(4-aminobenzamido)-2-(4-(((2,4-diaminopteridin-6-yl)methyl)(methyl) amino)-benzamido)pentanoic acid (XX12)

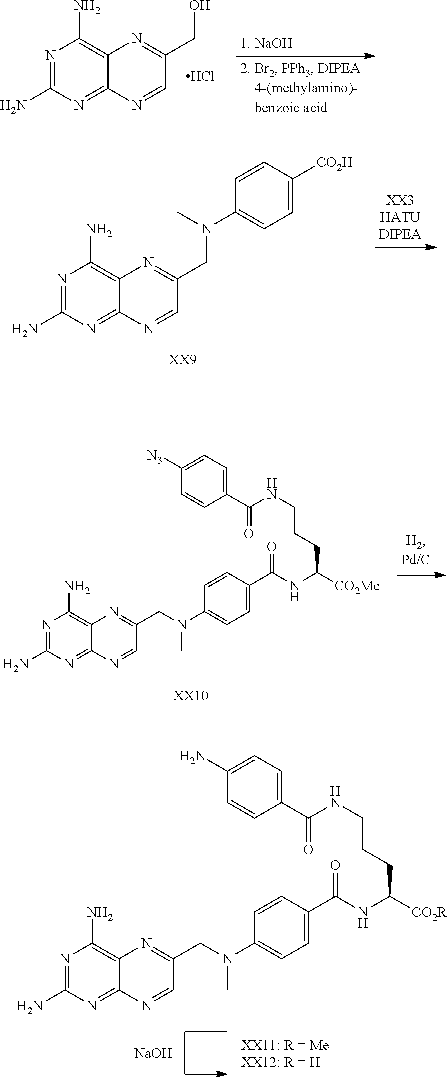

### 4-(((2,4-Diaminopteridin-6-yl)methyl)(methyl)amino)benzoic acid (XX9)

(2,4-Diaminopteridin-6-yl)methanol hydrochloride (8.80 g, 38.5 mmol) was dissolved in hot water (300 mL). After cooling to RT aq. NaOH (40 mL, 1.0 M) was added until pH>7. Filtered, washed with water (2×25 mL) and dried under vacuum at RT for 3 h. Residual water was azeotropically removed with ethanol (EtOH). The residue was triturated with hot EtOH and, after cooling to RT, filtered, washed with EtOH (50 mL) and ether (2×50 mL) and dried under vacuum to give (2,4-diaminopteridin-6-yl)methanol (6.41 g) as the free base. In a separate flask, bromine (1.60 mL, 31.2 mmol) was added dropwise to a cooled (0° C.) suspension of PPh₃ (8.19 g, 31.2 mmol) in dimethylacetamide (DMA; 13.5 mL) over 50 min. The addition rate was such that the internal temperature did not exceed 8° C. The thick slurry was stirred for 75 min at RT to give an orange slurry. Solid, free-base (2,4-diaminopteridin-6-yl)methanol (2.00 g, 10.4 mmol) was added and the temperature rose to 38° C. The mixture was stirred for 24 h at RT, then 4-(methylamino)benzoic acid (2.36 g, 15.6 mmol) was added followed by DIPEA (3.81 mL, 21.9 mmol). Stirred for 5 days and poured into aq. NaOH (136 mL, 0.33 M), using DMA (4 mL) to complete the transfer. Water (40 mL) was added and the precipitate was filtered off. The filtrate was acidified to pH 4.5 with 10% AcOH in water (ca. 25.0 mL). The precipitate was collected by filtration, washed with water, triturated with hot MeOH (16 mL), filtered and the residue suspended in dioxane and lyophilized to give acid XX9 (3.55 g, quant) as a yellow solid.

¹H NMR (400 MHz, DMSO-d₆) ppm=12.15 (br s, 1H), 8.65 (s, 1H), 8.35 (br s, 1H), 8.12 (br s, 1H), 7.73 (d, J=8.8 Hz, 2H), 7.20 (br s, 2H), 6.83 (d, J=8.8 Hz, 2H), 4.83 (s, 2H), 3.24 (s, 3H).

MS (ESI⁺) calc. for C₁₅H₁₆N₇O₂⁺ [M+H]⁺ 326.14, found 326.38.

### Methyl (S)-5-(4-azidobenzamido)-2-(4-(((2,4-diaminopteridin-6-yl)methyl)(methyl)amino) benzamido)-pentanoate (XX10)

Acid XX9 (250 mg, 0.768 mmol) was reacted with amine XX3 (323 mg, 1.11 mmol) according to general procedure XXA. Purification by flash chromatography (silica gel, MeOH:DCM 0:1 to 1:4) afforded ester XX10 (434 mg, 94%) as a yellow solid.

¹H NMR (400 MHz, DMSO-d₆) ppm=8.66 (d, J=7.5 Hz, 1H), 8.55 (s, 1H), 8.47 (t, J=5.6 Hz, 1H), 7.88 (d, J=8.6 Hz, 2H), 7.79 (d, J=8.2 Hz, 2H), 7.59 (br s, 2H), 7.34 (d, J=8.2 Hz, 2H), 7.19 (d, J=8.6 Hz, 2H), 6.54 (br s, 2H), 4.47-4.42 (m, 1H), 3.63 (s, 3H), 3.30-3.25 (m, 2H), 3.12 (s, 4H), 1.91-1.75 (m, 2H), 1.69-1.53 (m, 2H).

MS (ESI⁺) calc. for C₂₈H₃₀NₗₗO₄⁺ [M+H]⁺ 584.25, found 584.49.

### Methyl (S)-5-(4-aminobenzamido)-2-(4-(((2,4-diaminopteridin-6-yl)methyl)(methyl)amino) benzamido)-pentanoate (XX11)

Reduction of azide XX10 (195 mg, 0.326 mmol) was carried out according to general procedure XXB. Purification by flash chromatography (silica gel, MeOH:DCM 0:1 to 1:3) afforded aniline XX11 (111 mg, 60%) as a yellow solid.

¹H NMR (400 MHz, DMSO-d₆) ppm=8.56 (s, 1H), 8.34 (d, J=7.5 Hz, 1H), 7.96 (t, J=5.4 Hz, 1H), 7.73 (d, J=8.9 Hz, 2H), 7.67 (br s, 1H), 7.54 (d, J=8.6 Hz, 2H), 7.45 (br s, 1H), 6.81 (d, J=8.9 Hz, 2H), 6.62 (br s, 2H), 6.51 (d, J=8.6 Hz, 2H), 5.56 (br s, 2H), 4.78 (s, 2H), 4.41-4.35 (m, 1H), 3.60 (s, 3H), 3.24-3.18 (m, 2H), 3.21 (s, 3H), 1.85-1.70 (m, 2H), 1.64-1.46 (m, 2H).

MS (ESI⁺) calc. for C₂₈H₃₃N₁₀O₄⁺ [M+H]⁺ 573.27, found 573.52.

### (S)-5-(4-aminobenzamido)-2-(4-(((2,4-diaminopteridin-6-yl)methyl)(methyl)amino) benzamido)pentanoic acid (XX12)

The hydrolysis of ester XX11 (111 mg, 0.194 mmol) was carried out according to general procedure XXC, to give acid XX12 (72 mg, 66%) as a yellow solid.

¹H NMR (400 MHz, DMSO-d₆) ppm=12.43 (br s, 1H), 8.61 (s, 1H), 8.22 (d, J=7.8 Hz, 1H), 8.16 (br s, 1H), 7.97 (t, J=5.3 Hz, 1H), 7.94 (br s, 1H), 7.75 (d, J=8.7 Hz, 2H), 7.55 (d, J=8.5 Hz, 2H), 7.08 (br s, 2H), 6.81 (d, J=8.7 Hz, 2H), 6.51 (d, J=8.5 Hz, 2H), 6.04 (br s, 2H), 4.81 (s, 2H), 4.36-4.31 (m, 1H), 3.22 (s, 3H), 3.21-3.18 (m, 2H), 1.86-1.69 (m, 2H), 1.64-1.47 (m, 2H).

¹³C NMR (100 MHz, DMSO-d₆) ppm=174.1, 166.2, 166.2, 162.7, 160.7, 152.2, 151.4, 150.8, 149.0, 147.6, 129.0, 128.6, 121.6, 121.4, 121.3, 112.5, 111.1, 54.9, 52.4, 39.2, 38.6, 28.3, 26.5.

MS (ESI⁺) calc. for C₂₇H₃₁N₁₀O₄⁺ [M+H]⁺ 559.25, found 559.53.

### Preparation of methyl (S)-2-amino-5-(5-amino-1,3-dioxoisoindolin-2-yl)pentanoate (XT6)

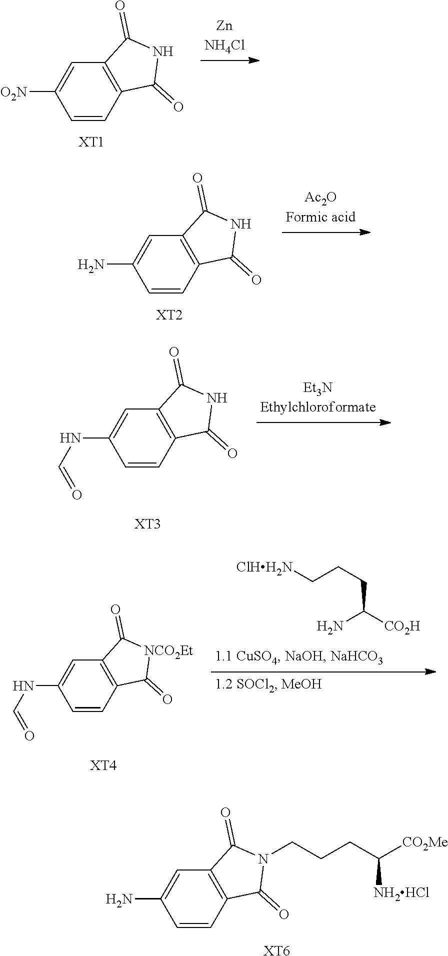

### 5-Aminoisoindoline-1,3-dione (XT2)

NH₄Cl (11.1 g, 208 mmol) in water (25 mL) was added to a solution of compound XT1 (10.0 g, 52.0 mmol) in THF (75 mL). Zinc dust (13.6 g, 208 mmol) was then carefully added portion-wise (CAUTION! exothermic) and the resulting suspension was stirred for 1 h. The yellow reaction mixture was filtered over Celite® and then rinsed with methanol (250 mL) and concentrated in vacuo to give crop 1. The Celite® cake was suspended in DMF (40 mL) and stirred overnight, then filtered and concentrated in vacuo to give crop 2. Crop 1 was stirred in water (50 mL) for 15 min, filtered and the cake washed with ether and dried on air overnight. Crop 2 was stirred in ether (150 mL) overnight, filtered and the solids stirred in water (100 mL) for 15 min. After filtration, the cake was washed with ether and the solid dried on air. Both batches were combined to give XT2 (12 g, quant) as a yellow solid.

MS (ESI⁺) calc. for C₈H₇N₂O₂⁺ [M+H]⁺ 163.05, found 163.06.

### N-(1,3-dioxoisoindolin-5-yl)formamide (XT3)

To a 250 mL round bottom flask loaded with FA (22.2 mL, 578 mmol) was slowly added Ac₂O (10.9 mL, 116 mmol) and the mixture was stirred for 10 min. Subsequently, finely ground aniline XT2 (3.75 g, 23.1 mmol) was added and the resulting mixture was stirred for 15 min at RT. The reaction mixture was concentrated in vacuo, water (25 mL) was added to the crude product and the resulting suspension stirred for 15 min. After filtration, the solids were dried on air overnight to yield formamide XT3 (2.70 g, 14.2 mmol, 61%) as a yellow solid.

¹H NMR (400 MHz, DMSO-d₆) ppm=11.24 (br s, 1H), 10.80 (s, 1H), 8.42 (d, J=1.1 Hz, 1H), 8.12 (d, J=1.1 Hz, 1H), 7.87 (dd, J=8.2, 1.1 Hz, 1H), 7.80 (d, J=8.2 Hz, 1H).

MS (ESI⁺) calc. for C₉H₇N₂O₃⁺ [M+H]⁺ 191.05, found 191.13.

### Ethyl 5-formamido-1,3-dioxoisoindoline-2-carboxylate (XT4)

To a cooled (0° C.) solution of formamide XT3 (2.50 g, 13.2 mmol) in DMF (25 mL), was added Et₃N (1.83 mL, 13.2 mmol), followed by the dropwise addition of ethyl chloroformate (1.25 mL, 13.2 mmol) in DMF (12.5 mL). The resulting mixture was stirred for 1 h at 0° C. More Et₃N (1.83 mL, 13.2 mmol) and ethyl chloroformate (1.25 mL, 13.2 mmol) in DMF (12.5 mL) were added (dropwise addition for the latter), and stirred at 0° C. for 30 min. Finally, Et₃N (0.92 mL, 6.6 mmol) and ethyl chloroformate (0.625 mL, 6.6 mmol) in DMF (6 mL) were added and stirred for a final 30 min at 0° C. The reaction mixture was concentrated in vacuo and the crude product was purified by flash chromatography (silica gel, EtOAc:DCM 0:1 to 1:0) to give formamide XT4 (1.4 g, 41%) as a yellow solid.

¹H NMR (400 MHz, DMSO-d₆) ppm=10.90 (br s, 1H), 8.45 (br s, 1H), 8.24 (br s, 1H), 7.95-7.90 (m, 2H), 4.36 (q, J=7.1 Hz, 2H), 1.32 (t, J=7.1 Hz, 3H).

MS (ESI⁺) calc. for C₁₂H₁₁N₂O₅⁺ [M+H]⁺ 263.07, found 263.14.

### Methyl (S)-2-amino-5-(5-amino-1,3-dioxoisoindolin-2-yl)pentanoate (XT6)

To a solution of L-Orn-OH·HCl (0.835 g, 4.85 mmol) and NaOH (0.388 g, 9.71 mmol) in water (9 mL) at RT, was added a solution of CuSO₄·5H₂O (0.606 g, 2.43 mmol) in water (9 mL) resulting in a deep blue solution. To this solution were added NaHCO₃ (0.489 g, 5.82 mmol) and ground formamide XT4 (1.40 g, 5.34 mmol) and the light blue suspension was stirred for 4 h at RT. The mixture was filtered and the solids were washed with water (2×2.5 mL), ethanol (2×2.5 mL) and ether (2×2.5 mL) and air dried overnight to give a yellow/grey copper-salt (1.6 g). The material was suspended in methanol (45 mL) and cooled to −20° C. Thionyl chloride (4.21 mL, 57.7 mmol) was added over a 45-min period while maintaining the temperature under 0° C. After warming to RT and stirring for 18 h, the reaction mixture was concentrated in vacuo and coevaporated with toluene (12 mL). The crude residue was triturated with a mixture of MeOH (4 mL)/EtOAc (3.6 mL)/acetone (3.6 mL), the solids filtered, washed with ether (20 mL) and dried under vacuum to yield succinimide XT6 (1.50 g, 87%) as a yellow solid.

¹H NMR (400 MHz, DMSO-d₆) ppm=8.57 (br s, 3H), 7.50 (d, J=8.1 Hz, 1H), 7.32 (br s, 3H), 6.98 (s, 1H), 6.85 (d, J=8.0 Hz, 1H), 4.05-3.97 (m, 1H), 3.71 (s, H), 3.55-3.47 (m, 2H), 1.83-1.54 (m, 4H).

MS (ESI⁺) calc. for C₁₄H₁₈N₃O₄⁺ [M+H]⁺ 292.13, found 292.17.

### Preparation of (S)-4-amino-2-((4-carboxy-4-(4-(((2,4-diaminopteridin-6-yl)methyl)amino) benzamido)butyl)carbamoyl)-benzoic acid (XT9) and (S)-5-amino-2-((4-carboxy-4-(4-(((2,4-diaminopteridin-6-yl)methyl)-amino)benzamido)butyl)carbamoyl)benzoic acid (XT10)

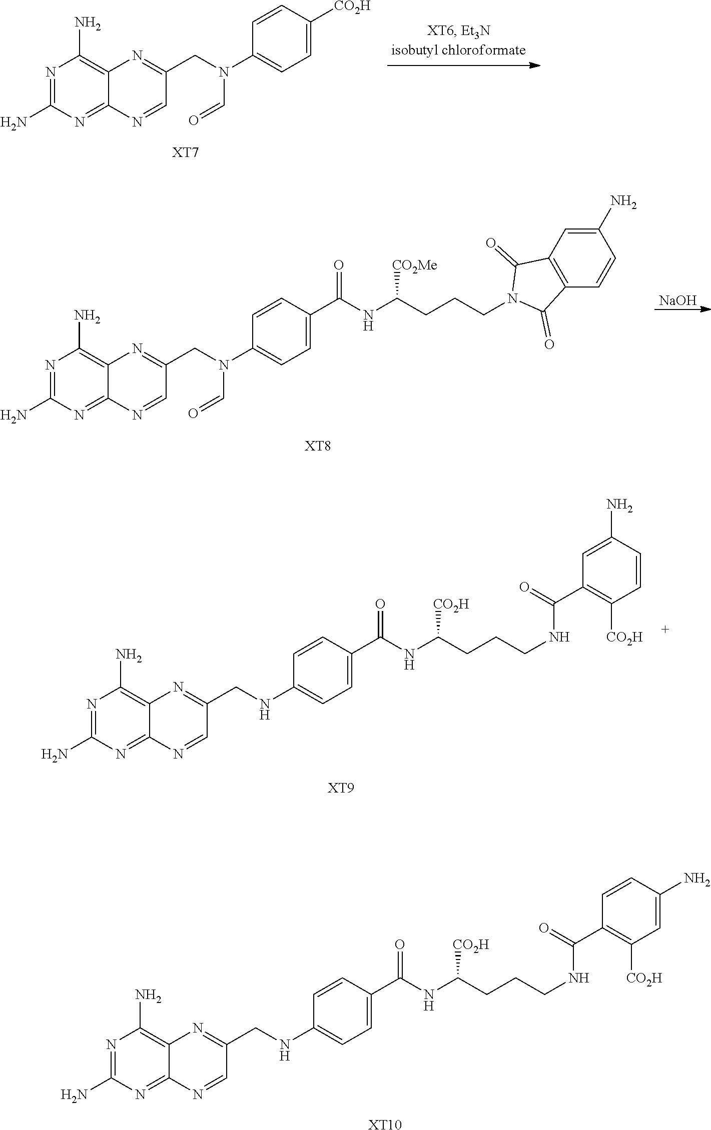

### Methyl (S)-5-(5-amino-1,3-dioxoisoindolin-2-yl)-2-(4-(N-((2,4-diaminopteridin-6-yl)methyl)-formamido)benzamido)pentanoate (XT8)

To a suspension of acid XT7 (470 mg, 1.39 mmol; synthesized as described in US 2004/0072837) and Et₃N (2.32 mL, 16.6 mmol) in DMF (10 mL) was added isobutyl chloroformate (0.182 mL, 1.385 mmol) at RT. The resulting mixture was stirred for 1 h, then succinimide XT6 (499 mg, 1.52 mmol) was added and stirred for 1 h. More isobutyl chloroformate (0.091 mL, 0.692 mmol) was added, followed after 20 min by succinimide XT6 (250 mg, 0.762 mmol) and the mixture was stirred for 1 h. Finally, more isobutyl chloroformate (0.045 mL, 0.347 mmol) was added, followed after 20 min by succinimide XT6 (125 mg, 0.381 mmol) and the mixture stirred for 1 h. After concentration, the crude product was purified by flash chromatography (silica gel, MeOH:DCM 0:1 to 20:80). The product was suspended in water (6 mL), filtered, and washed with water (2 mL) and Et₂O (4 mL) to yield formamide XT8 (552 mg, 65%) as a yellow solid.

¹H NMR (400 MHz, DMSO-d₆) ppm=8.81 (s, 1H), 8.74 (br s, 1H), 8.69 (d, J=7.3 Hz, 1H), 8.01 (br s, 1H), 7.87 (d, J=8.6 Hz, 2H), 7.72 (br s, 1H), 7.60 (d, J=8.6 Hz, 2H), 7.47 (d, J=8.2 Hz, 1H), 6.97 (br s, 2H), 6.91 (d, J=1.8 Hz, 1H), 6.78 (dd, J=8.2, 1.8 Hz, 1H), 6.45 (br s, 2H), 5.25 (s, 2H), 4.46-4.37 (m, 1H), 3.61 (s, 3H), 3.51 (t, J=6.4 Hz, 2H), 1.83-1.46 (m, 4H).

MS (ESI⁺) calc. for C₂₉H₂₉N₁₀O₆⁺ [M+H]⁺ 613.23, found 613.30.

### (S)-4-Amino-2-((4-carboxy-4-(4-(((2,4-diaminopteridin-6-yl)methyl)amino)benzamido) butyl)carbamoyl)-benzoic acid (XT9) and (S)-5-amino-2-((4-carboxy-4-(4-(((2,4-diaminopteridin-6-yl)methyl)-amino)benzamido)butyl)carbamoyl)benzoic acid (XT10)

The hydrolysis of formamide XT8 (120 mg, 0.196 mmol) with NaOH (6 eq) was carried out according to general procedure XXC. The solids were taken up in 10% aq. MeCN containing 0.1% TFA and purified by preparative RP-HPLC (water×0.1% TFA/MeCN×0.1% TFA, gradient 5% to 35%). Product fractions were pooled, MeCN removed by rotary evaporation and the aq. solution was lyophilized to yield acid XT9 (25 mg, 22%) and acid XT10 (5 mg, 4% yield) as yellow solids.

XT9: ¹H NMR (400 MHz, DMSO-d₆) ppm=12.44 (br s, 1H), 9.31 (br s, 1H), 9.26 (br s, 1H), 8.57 (br s, 1H), 8.14 (d, J=7.7 Hz, 1H), 7.99 (t, J=5.6 Hz, 1H), 7.80-7.63 (m, 2H) 7.74 (d, J=8.6 Hz, 1H), 7.56 (d, J=8.5 Hz, 1H), 6.90 (br s, 1H), 6.75 (d, J=2.2 Hz, 1H), 6.52 (dd, J=8.5, 2.2 Hz, 1H), 6.41 (d, J=2.2 Hz, 1H), 5.91 (br s, 2H), 4.61 (s, 2H), 4.40-4.29 (m, 1H), 3.20-3.10 (m, 2H), 2.84-2.76 (m, 2H), 1.94-1.70 (m, 2H), 1.67-1.47 (m, 2H).

MS (ESI⁺) calc. for C₂₇H₂₉N₁₀O₆⁺ [M+H]⁺ 589.23, found 589.37.

XT10: ¹H NMR (400 MHz, DMSO-d₆) ppm=12.53 (br s, 2H), 9.34 (br s, 1H), 9.30 (br s, 1H), 8.84 (s, 1H), 8.62 (br s, 1H), 8.17 (d, J=7.6 Hz, 1H), 8.07 (t, J=5.5 Hz, 1H), 7.79 (br s, 1H), 7.74 (d, J=8.7 Hz, 2H), 7.19 (d, J=8.2 Hz, 1H), 7.07-6.70 (m, 1H) 6.82 (d, J=2.2 Hz, 1H), 6.74 (d, J=8.6 Hz, 2H), 6.61 (dd, J=8.3, 2.3 Hz, 1H), 4.62 (s, 2H), 4.37-4.29 (m, 1H), 3.16 (q, J=5.9 Hz 2H) 1.91-1.69 (m, 2H), 1.67-1.47 (m, 2H).

MS (ESI⁺) calc. for C₂₇H₂₉N₁₀O₆⁺ [M+H]⁺ 589.23, found 589.47.

### Preparation of 2-(((S)-4-carboxy-4-(4-(((2,4-diaminopteridin-6-yl)methyl)amino) benzamido)-butyl)carbamoyl)-4-((S)-2-((S)-2-(6-(2,5-dioxo-2,5-dihydro-1H-pyrrol-1-yl) hexanamido)-3-methyl-butanamido)propanamido)benzoic acid (XT16) and 2-(((S)-4-carboxy-4-(4-(((2,4-diaminopteridin-6-yl)methyl)amino)benzamido)butyl)carbamoyl)-5-((S)-2-((S)-2-(6-(2,5-dioxo-2,5-dihydro-1H-pyrrol-1-yl)hexanamido)-3-methylbutanamido) propanamido)benzoic acid (XT17)

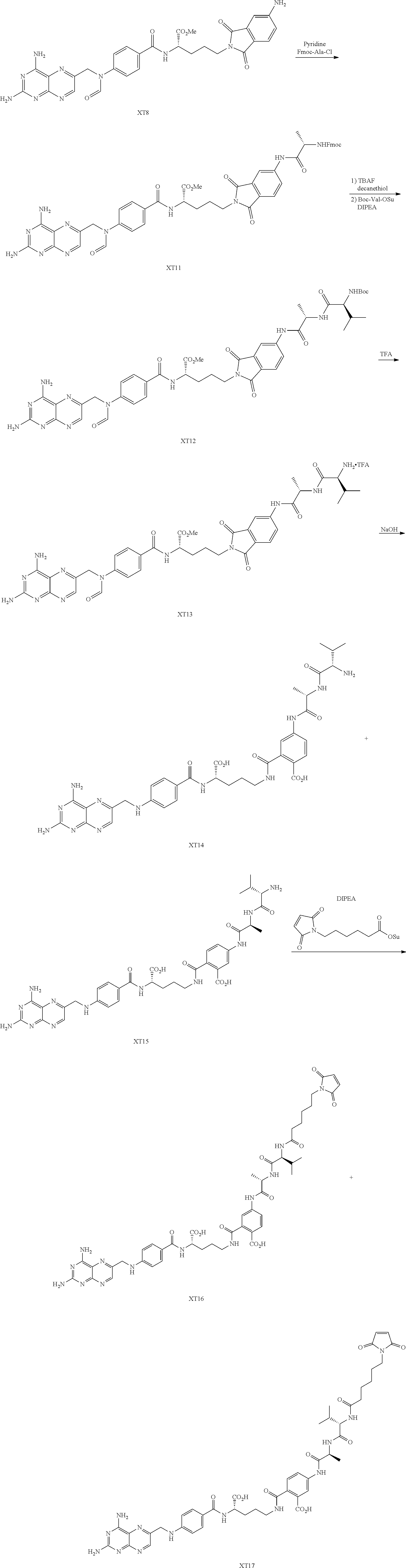

### Methyl (S)-5-(5-((S)-2-((((9H-fluoren-9-yl)methoxy)carbonyl)amino)propanamido)-1,3-dioxoisoindolin-2-yl)-2-(4-(N-((2,4-diaminopteridin-6-yl)methyl)formamido)benzamido) pentanoate (XT11)

To a suspension of succinimide XT8 (170 mg, 0.278 mmol) in pyridine (5 mL) at 0° C., was added (9H-fluoren-9-yl)methyl (S)-(1-chloro-1-oxopropan-2-yl)carbamate (Fmoc-Ala-Cl, 320 mg, 0.971 mmol) (prepared according to Unsworth et al, Angew. Chem. Int. Ed. 2015, 52, 15794-15798) divided over six portions. Once UPLC analysis of MeOH quenched samples indicated full conversion, the reaction mixture was quenched with MeOH (6 mL) and concentrated in vacuo. The crude product was purified by flash chromatography (silica gel, MeOH:DCM 0:1 to 20:80) to yield amide XT11 (298 mg, quant) as a yellow solid.

¹H NMR (400 MHz, DMSO-d₆) ppm=10.71 (s, 1H), 9.07 (br s, 1H), 8.81 (s, 1H), 8.79 (br s, 1H), 8.71 (d, J=7.7 Hz, 1H), 8.21 (s, 1H), 7.93-7.84 (m, 4H), 7.83-7.78 (m, 2H), 7.74 (t, J=6.7 Hz, 2H), 7.60 (d, J=8.6 Hz, 2H), 7.45-7.38 (m, 2H), 7.37-7.30 (m, 2H), 5.30 (s, 2H), 4.47-4.38 (m, 1H), 4.29 (d, J=6.9 Hz, 2H), 4.22 (t, J=6.6 Hz, 2H), 4.09 (br s, 4H), 3.64-3.52 (m, 5H), 1.86-1.60 (m, 4H), 1.34 (d, J=7.1 Hz, 3H).

¹³C NMR (100 MHz, DMSO-d₆) ppm=173.2, 173.0, 168.2, 168.1, 166.2, 163.1, 156.4, 145.0, 144.3, 144.2, 144.0, 141.2, 133.6, 131.2, 129.2, 128.1, 127.5, 126.0, 125.8, 124.7, 123.8, 122.2, 120.6, 113.3, 89.8, 66.1, 52.8, 52.4, 51.1, 47.1, 37.5, 28.3, 25.4, 18.2.

MS (ESI⁺) calc. for C₄₇H₄₄N₁₁O₉⁺ [M+H]⁺ 906.33 found 906.40.

### Methyl (S)-5-(5-((S)-2-((S)-2-((tert-butoxycarbonyl)amino)-3-methylbutanamido) propanamido)-1,3-dioxoisoindolin-2-yl)-2-(4-(N-((2,4-diaminopteridin-6-yl)methyl) formamido)benzamido)pentanoate (XT12)

A 25 mL round bottom flask was charged with amide XT11 (250 mg, 0.276 mmol) and tetrabutylammoniumfluoride trihydrate (TBAF·3H₂O) (178 mg, 0.552 mmol), and the flask was thoroughly purged with N₂ (minimum of 3 vacuum/N₂ cycles). DMF (6 mL) was added and once all solids had dissolved, decanethiol (0.608 mL, 2.76 mmol) was added directly into the solution via syringe at RT, and the mixture was stirred for 90 min. Next, Boc-Val-OSu (130 mg, 0.414 mmol) and DIPEA (0.096 mL, 0.552 mmol) were added and the resulting mixture was stirred for 1.5 h. A second portion of Boc-Val-OSu (130 mg, 0.414 mmol) was added and the mixture was stirred for an additional 30 min. The reaction mixture was subsequently concentrated in vacuo and the crude product was purified by flash chromatography (silica gel, MeOH:DCM 0:1 to 20:80) to give dipeptide XT12, contaminated with tetrabutylammonium salts.

MS (ESI⁺) calc. for C₄₂H₅₁N₁₂O₁₀⁺ [M+H]⁺ 883.38, found 883.71.

### Methyl (S)-5-(5-((S)-2-((S)-2-amino-3-methylbutanamido)propanamido)-1,3-dioxoisoindolin-2-yl)-2-(4-(N-((2,4-diaminopteridin-6-yl)methyl)formamido)benzamido) pentanoate (XT13)

To a solution of impure dipeptide XT12 (244 mg, 0.276 mmol) in DCM (3 mL) at 0° C. was added TFA (3 mL). The resulting mixture was stirred for 15 min whilst warming to RT. The reaction mixture was concentrated in vacuo and coevaporated with DCM to yield crude deprotected amine XT13 as an orange TFA salt which was used directly without further analysis.

MS (ESI⁺) calc. for C₃₇H₄₃N₁₂O₈⁺ [M+H]⁺ 783.33, found 783.32

### 4-((S)-2-((S)-2-Amino-3-methylbutanamido)propanamido)-2-(((S)-4-carboxy-4-(4-(((2,4-diaminopteridin-6-yl)methyl)amino)benzamido)butyl)carbamoyl)benzoic acid (XT14) and 5-((S)-2-((S)-2-amino-3-methylbutanamido)propanamido)-2-(((S)-4-carboxy-4-(4-(((2,4-diaminopteridin-6-yl)methyl)amino)benzamido)butyl)carbamoyl)benzoic acid (XT15)

The hydrolysis of formamide XT13 (190 mg, 0.243 mmol) was carried out according to general procedure XXC, with the following modifications; formamide XT13 was first reacted with NaOH (12 eq) at 0° C. for 1 h, and then after addition of a second portion of NaOH (12 eq), for 6 h at RT. Washing of the solid with MeOH was replaced with washing with water (3 mL). A mixture of diacids XT14 and XT15 (˜1:2 ratio) was obtained. Optionally, these can be purified by preparative RP-HPLC (water×0.10% TFA/MeCN×0.10% TFA, gradient 5% to 35%), to afford diacids XT14 (15 mg, 8%, 3 steps) and XT15 (30 mg, 16%, 3 steps) as yellow solids.

XT14: ¹H NMR (400 MHz, DMSO-d₆) ppm=13.37-12.23 (m, 2H), 10.46 (s, 1H), 8.96 (br s, 1H), 8.81 (s, 1H), 8.75 (d, J=6.7 Hz, 1H), 8.22 (t, J=5.4 Hz, 1H), 8.15 (d, J=7.8 Hz, 1H), 8.08 (br s, 2H), 7.80 (d, J=8.5 Hz, 1H), 7.74 (d, J=8.6 Hz, 2H), 7.67 (dd, J=8.5, 1.7 Hz, 1H), 7.58 (d, J=1.7 Hz, 1H), 6.91-6.86 (m, 1H), 6.75 (d, J=8.6 Hz, 2H), 6.54 (s, 2H), 4.59 (s, 2H), 4.49 (p, J=6.9 Hz, 1H), 4.38-4.30 (m, 1H), 3.61 (s, 1H), 3.17 (q, J=5.6 Hz, 2H), 2.13-2.04 (m, 1H), 1.93-1.83 (m, 1H), 1.83-1.71 (m, 1H), 1.69-1.49 (m, 2H), 1.36 (d, J=7.0 Hz, 3H), 0.95 (t, J=6.2 Hz, 6H).

MS (ESI⁺) calc. for C₃₅H₄₃N₁₂O₈⁺ [M+H]⁺ 759.33, found 759.50.

XT15: ¹H NMR (400 MHz, DMSO-d₆) ppm=13.20-12.32 (m, 2H), 10.38 (s, 1H), 9.01 (br s, 1H), 8.81 (s, 1H), 8.74 (d, J=6.8 Hz, 1H), 8.24 (t, J=5.4 Hz, 1H), 8.16 (d, J=7.7 Hz, 1H), 8.08 (br s, 2H), 7.97 (d, J=2.0 Hz, 1H), 7.77-7.69 (m, 3H), 7.39 (d, J=8.3 Hz, 1H), 6.91-6.86 (m, 1H), 6.75 (d, J=8.7 Hz, 2H), 6.54 (br s, 2H), 4.60 (d, J=3.4 Hz, 2H), 4.48 (p, J=7.0 Hz, 1H), 4.39-4.31 (m, 1H), 3.62 (s, 1H), 3.19 (q, J=5.9 Hz, 2H), 2.15-2.04 (m, 1H), 1.93-1.83 (m, 1H), 1.83-1.72 (m, 1H), 1.68-1.50 (m, 2H), 1.37 (d, J=7.1 Hz, 3H), 0.96 (t, J=6.1 Hz, 6H).

MS (ESI⁺) calc. for C₃₅H₄₃N₁₂O₈⁺ [M+H]⁺ 759.33, found 759.68.

### 2-(((S)-4-Carboxy-4-(4-(((2,4-diaminopteridin-6-yl)methyl)amino)benzamido)butyl) carbamoyl)-4-((S)-2-((S)-2-(6-(2,5-dioxo-2,5-dihydro-1H-pyrrol-1-yl)hexanamido)-3-methylbutanamido)propanamido)benzoic acid (XT16) and 2-(((S)-4-carboxy-4-(4-(((2,4-diaminopteridin-6-yl)methyl)amino)benzamido)-butyl)carbamoyl)-5-((S)-2-((S)-2-(6-(2,5-dioxo-2,5-dihydro-1H-pyrrol-1-yl)hexanamido)-3-methyl-butanamido)propanamido)benzoic acid (XT17)

To a cooled (0° C.) mixture (˜1:2) of diacids XT14 and XT15 (0.209 g, 0.276 mmol) in DMF (10 mL) was added 6-maleimidohexanoic acid N-hydroxylsuccinimide ester (0.085 g, 0.276 mmol) followed by DIPEA (0.289 mL, 1.66 mmol). The resulting mixture was stirred for 3 h at RT and was subsequently concentrated in vacuo. The crude solid was purified by preparative RP-HPLC (water×0.10% TFA/MeCN×0.1% TFA, gradient 20% to 50%). Product fractions were pooled, MeCN was removed by rotary evaporation, and the aqueous solution was lyophilized to yield a mixture (1:2) of XT16 and XT17 (65 mg, 25%, 4 steps) as a yellow solid. Conducting the reaction with isomerically pure XT14 or XT15 allowed for the isolation of pure XT16 or XT17, respectively.

XT16: ¹H NMR (400 MHz, DMSO-d₆) ppm=13.27-12.28 (m, 3H), 10.15 (s, 1H), 9.31 (br s, 1H), 9.27 (br s, 1H), 8.84 (s, 1H), 8.57 (br s, 1H), 8.26-8.18 (m, 2H), 8.16 (d, J=7.7 Hz, 1H), 7.97 (d, J=2.0 Hz, 1H), 7.80 (d, J=8.5 Hz, 1H), 7.76-7.71 (m, 3H), 7.60 (br s, 1H), 7.37 (d, J=8.4 Hz, 1H), 7.00 (s, 2H), 6.89 (br s, 1H), 6.75 (d, J=8.7 Hz, 2H), 6.53 (br s, 1H), 4.62 (s, 2H), 4.41-4.30 (m, 2H), 4.17 (dd, J=8.2, 7.2 Hz, 1H), 3.19 (q, J=6.2 Hz, 2H), 2.23-2.06 (m, 2H), 2.01-1.72 (m, 3H), 1.68-1.42 (m, 6H), 1.31 (d, J=7.0 Hz, 3H), 1.25-1.13 (m, 2H), 0.84 (dd, J=16.7, 6.8 Hz, 6H).

MS (ESI⁺) calc. for C₄₅H₅₄N₁₃O₁₁⁺ [M+H]⁺ 952.41, found 952.75.

XT17: (31 mg, 82%) as a yellow solid. ¹H NMR (400 MHz, DMSO-d₆) ppm=13.31-12.29 (m, 2H), 10.25 (s, 1H), 9.29 (br s, 1H), 9.25 (br s, 1H), 8.83 (s, 1H), 8.56 (br s, 1H), 8.26-8.18 (m, 2H), 8.15 (d, J=7.8 Hz, 1H), 7.79 (d, J=8.7 Hz, 1H), 7.78 (d, J=8.5 Hz, 1H), 7.74 (d, J=8.7 Hz, 2H), 7.69 (dd, J=8.7, 1.9 Hz, 1H), 7.59 (d, J=1.9 Hz, 1H), 7.00 (s, 2H), 6.89 (br s, 1H), 6.75 (d, J=8.7 Hz, 2H), 6.54 (br s, 1H), 4.62 (s, 2H), 4.40-4.30 (m, 2H), 4.17 (dd, J=8.3, 7.3 Hz, 1H), 3.38 (q, J=5.9 Hz, 2H), 2.23-2.05 (m, 2H), 1.98-1.71 (m, 3H), 1.68-1.41 (m, 6H), 1.31 (d, J=7.2 Hz, 3H), 1.25-1.13 (m, 2H), 0.84 (dd, J=17.7, 6.7 Hz, 6H).

MS (ESI⁺) calc. for C₄₅H₅₄N₁₃O₁₁⁺ [M+H]⁺ 952.41, found 952.82.

### Preparation of (S)-4-amino-2-((4-carboxy-4-(4-(((2,4-diaminopteridin-6-yl)methyl)(methyl) amino)-benzamido)butyl)-carbamoyl)benzoic acid (XT20) and (S)-5-amino-2-((4-carboxy-4-(4-(((2,4-diaminopteridin-6-yl)methyl)(methyl)amino)benzamido)butyl) carbamoyl)benzoic acid (XT21)

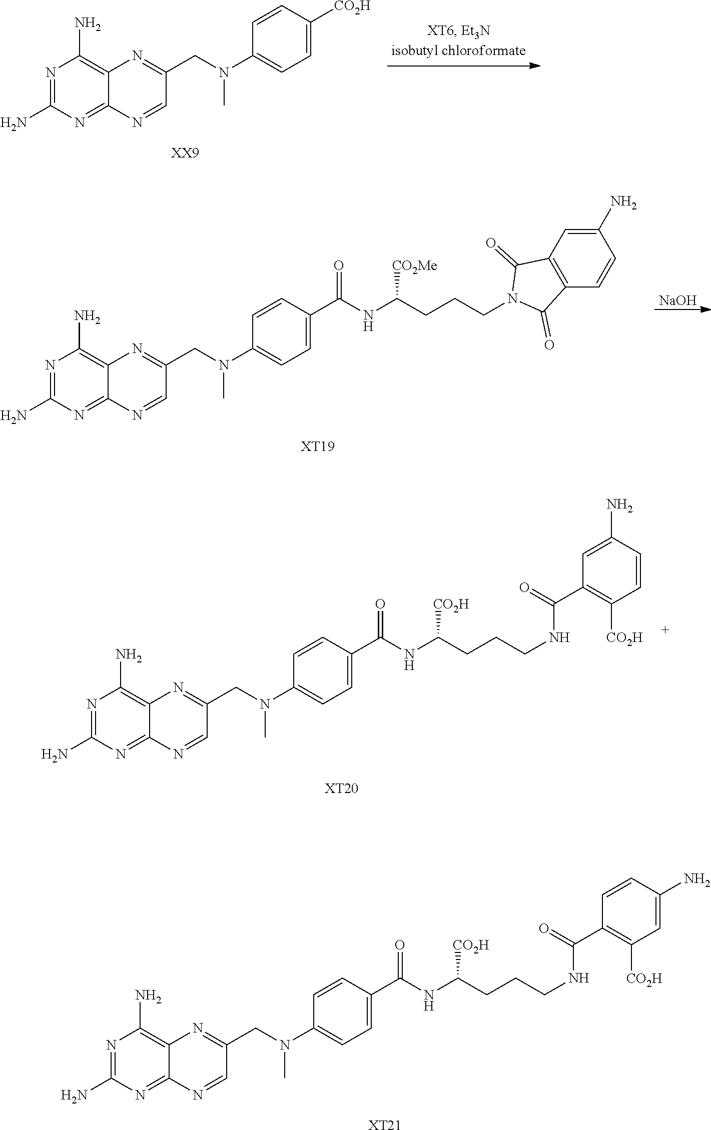

### Methyl (S)-5-(5-amino-1,3-dioxoisoindolin-2-yl)-2-(4-(((2,4-diaminopteridin-6-yl)methyl) (methyl)-amino)benzamido)pentanoate (XT19)

To a suspension of acid XX9 (150 mg, 0.461 mmol) and triethylamine (1.16 mL, 8.30 mmol) in DMF (5 mL) at RT was added isobutyl chloroformate (0.061 mL, 0.461 mmol), and the reaction was stirred for 1 h. Amine XT6 (166 mg, 0.507 mmol) was added and the reaction was stirred for 1 h. More isobutyl chloroformate (0.030 mL, 0.230 mmol) was added and after 20 min more amine XT6 (83 mg, 0.254 mmol) was added. A third, and final portion of isobutyl chloroformate (0.015 mL, 0.115 mmol) was added, followed after 20 min by the addition of more XT6 (42 mg, 0.127 mmol). Despite multiple additions of chloroformate, the reaction stalled at 30% conversion. HATU (175 mg, 0.461 mmol) was added at RT for 30 min, followed by the addition of amine XT6 (166 mg, 0.507 mmol). After 30 min, the reaction mixture was concentrated in vacuo and the crude product was purified by flash chromatography (silica gel, MeOH:DCM 0:1 to 25:75) to yield aniline XT19 (294 mg, quant) as a yellow solid.

MS (ESI⁺) calc. for C₂₉H₃₁N₁₀O₅⁺ [M+H]⁺ 599.25, found 599.29.

### (S)-4-Amino-2-((4-carboxy-4-(4-(((2,4-diaminopteridin-6-yl)methyl)(methyl)amino) benzamido)butyl)-carbamoyl)benzoic acid (XT20) and (S)-5-amino-2-((4-carboxy-4-(4-(((2,4-diaminopteridin-6-yl)methyl)(methyl)amino)benzamido)butyl)carbamoyl)benzoic acid (XT21)

The hydrolysis of ester XT19 (276 mg, 0.461 mmol) with NaOH (12 eq) was carried out according to general procedure XXC, with the modification that upon collection of the solid by filtration only ether was used to wash the solid. The crude was purified by preparative RP-HPLC (water×0.1% TFA/MeCN×0.1% TFA, gradient 5% to 35%). Product fractions were pooled, MeCN was removed by rotary evaporation, and the aqueous solution was lyophilized to yield diacids XT20 (10 mg, 0.017 mmol, 4%) and XT21 (76 mg, 0.126 mmol, 27%) as yellow solids.

XT20: ¹H NMR (400 MHz, DMSO-d₆) ppm=12.35 (br s, 2H), 9.26 (s, 1H), 9.05 (s, 1H), 8.72 (s, 1H), 8.58 (br s, 1H), 8.19 (dd, J=7.7 Hz, 1H), 7.99 (t, J=5.6 Hz, 1H), 7.76 (d, J=8.8 Hz, 1H), 7.75-7.67 (m, 1H), 7.56 (d, J=8.5 Hz, 1H), 6.82 (d, J=8.8 Hz, 2H), 6.63-6.53 (m, 1H), 6.52 (dd, J=8.5, 2.2 Hz, 1H), 6.40 (d, J=2.2 Hz, 1H), 4.87 (s, 2H), 4.40-4.27 (m, 1H), 3.25 (s, 3H), 3.14 (q, J=5.8 Hz, 2H), 1.93-1.70 (m, 2H), 1.67-1.46 (m, 2H).

MS (ESI⁺) calc. for C₂₈H₃₁N₁₀O₆⁺ [M+H]⁺ 603.24, found 603.40.

XT21: ¹H NMR (400 MHz, DMSO-d₆) ppm=9.27 (s, 1H), 9.07 (s, 1H), 8.72 (s, 1H), 8.61 (br s, 1H), 8.21 (d, J=7.7 Hz, 1H), 8.07 (t, J=5.6 Hz, 1H), 7.86 (br s, 1H), 7.76 (d, J=8.8 Hz, 2H), 7.20 (d, J=8.3 Hz, 1H), 6.85-6.79 (m, 3H), 6.62 (dd, J=8.3, 2.2 Hz, 1H), 4.87 (s, 2H), 4.39-4.28 (m, 1H), 3.25 (s, 3H), 3.16 (q, J=6.1 Hz, 2H), 1.93-1.70 (m, 2H), 1.66-1.46 (m, 2H).

MS (ESI⁺) calc. for C₂₈H₃₁N₁₀O₆⁺ [M+H]⁺ 603.24, found 603.27.

### Preparation of (S)-4-amino-2-((4-carboxy-4-(4-(2-(2,4-diaminopteridin-6-yl)ethyl) benzamido)-butyl)carbamoyl)benzoic acid (XT24) and (S)-5-amino-2-((4-carboxy-4-(4-(2-(2,4-diaminopteridin-6-yl)ethyl)benzamido)butyl)-carbamoyl)benzoic acid (XT25)

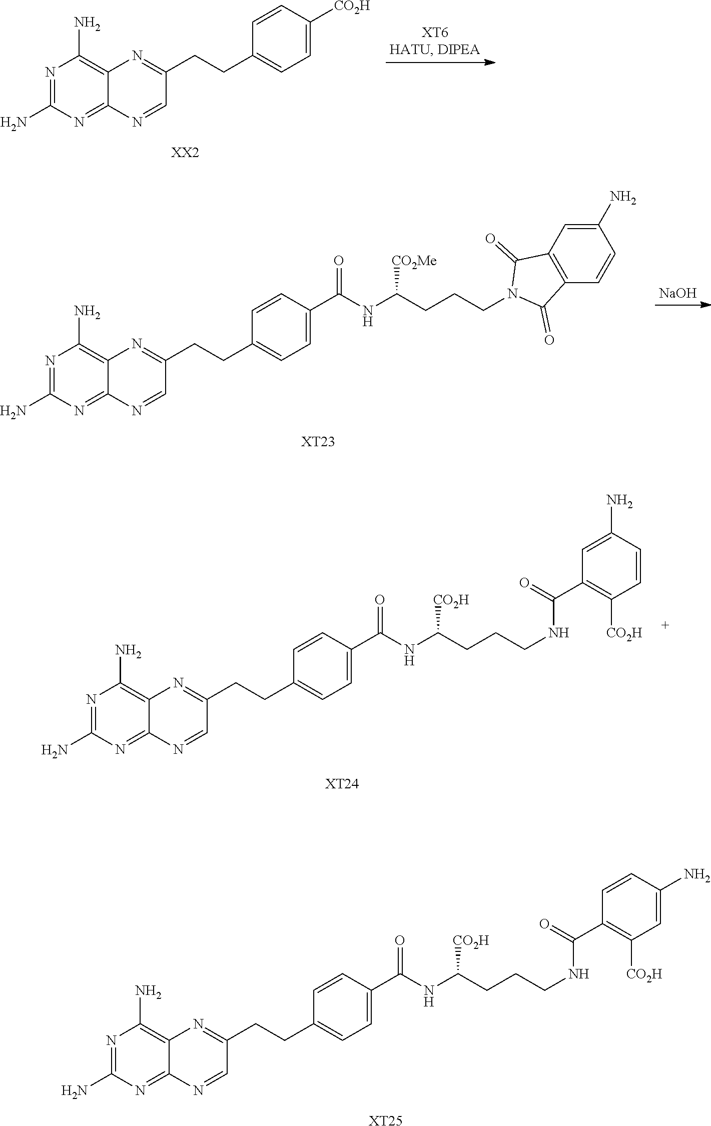

### Methyl (S)-5-(5-amino-1,3-dioxoisoindolin-2-yl)-2-(4-(2-(2,4-diaminopteridin-6-yl)ethyl) benzamido)-pentanoate (XT23)

Acid XX2 (140 mg, 0.451 mmol) was reacted with amine XT6 (222 mg, 0.677 mmol) according to general procedure XXA. Purification by flash chromatography (silica gel, MeOH:DCM 0:1 to 1:3) afforded ester XT23 (263 mg, quant) as a yellow solid.

MS (ESI⁺) calc. for C₂₉H₃₀N₉O₅⁺ [M+H]⁺ 584.24, found 584.24.

### (S)-4-Amino-2-((4-carboxy-4-(4-(2-(2,4-diaminopteridin-6-yl)ethyl)benzamido)butyl) carbamoyl)benzoic acid (XT24) and (S)-5-amino-2-((4-carboxy-4-(4-(2-(2,4-diaminopteridin-6-yl)ethyl)benzamido)butyl)-carbamoyl)benzoic acid (XT25)

The hydrolysis of ester XT23 (263 mg, 0.451 mmol) with NaOH (3 eq) was carried out according to general procedure XXC, with the modification that upon collection of the solid by filtration only ether was used to wash the solid. The crude was purified by preparative RP-HPLC (water×0.1% TFA/MeCN×0.1% TFA, gradient 5% to 35%). Product fractions were pooled, MeCN was removed by rotary evaporation, and the aqueous solution was lyophilized to yield diacids XT24 (7 mg, 0.012 mmol, 3%) and XT25 (93 mg, 0.158 mmol, 35%) as colorless solids.

XT24: ¹H NMR (400 MHz, DMSO-d₆) ppm=13.40-11.80 (m, 2H), 9.22 (br s, 1H), 8.75 (s, 1H), 8.61-8.47 (m, 1H), 8.50 (d, J=7.7 Hz, 1H), 8.01 (t, J=5.6 Hz, 1H), 7.83 (d, J=8.2 Hz, 2H), 7.77-7.65 (m, 1H), 7.56 (d, J=8.5 Hz, 1H), 7.36 (d, J=8.3 Hz, 2H), 6.67-6.43 (m, 4H), 6.52 (dd, J=8.5, 2.2 Hz, 1H), 6.41 (d, J=2.3 Hz, 1H), 4.43-4.32 (m, 1H), 3.29-3.12 (m, 6H), 1.95-1.72 (m, 2H), 1.69-1.49 (m, 2H).

MS (ESI⁺) calc. for C₂₈H₃₀N₉O₆⁺ [M+H]⁺ 588.23, found 588.46.

XT25: ¹H NMR (400 MHz, DMSO-d₆) ppm=9.25 (br s, 1H), 9.16 (br s, 1H), 8.75 (s, 1H), 8.64-8.50 (m, 1H), 8.55 (d, J=7.7 Hz, 1H), 8.09 (t, J=5.5 Hz, 1H), 7.93 (br s, 1H), 7.83 (d, J=8.2 Hz, 2H), 7.36 (d, J=8.2 Hz, 2H), 7.21 (d, J=8.3 Hz, 1H), 6.85 (d, J=2.3 Hz, 1H), 6.64 (dd, J=8.3, 2.3 Hz, 1H), 4.41-4.33 (m, 1H), 3.30-3.12 (m, 6H), 1.95-1.72 (m, 2H), 1.67-1.49 (m, 2H).

MS (ESI⁺) calc. for C₂₈H₃₀N₉O₆⁺ [M+H]⁺ 588.23, found 588.48.

### Preparation of (S)-2-((4-carboxy-4-(4-(((2,4-diaminopteridin-6-yl)methyl)amino)-benzamido)butyl)carbamoyl)-4-(6-(2,5-dioxo-2,5-dihydro-1H-pyrrol-1-yl)hexanamido) benzoic acid (XT31) and (S)-2-((4-carboxy-4-(4-(((2,4-diaminopteridin-6-yl)methyl)amino) benzamido)butyl)carbamoyl)-5-(6-(2,5-dioxo-2,5-dihydro-1H-pyrrol-1-yl)hexanamido) benzoic acid (XT32)

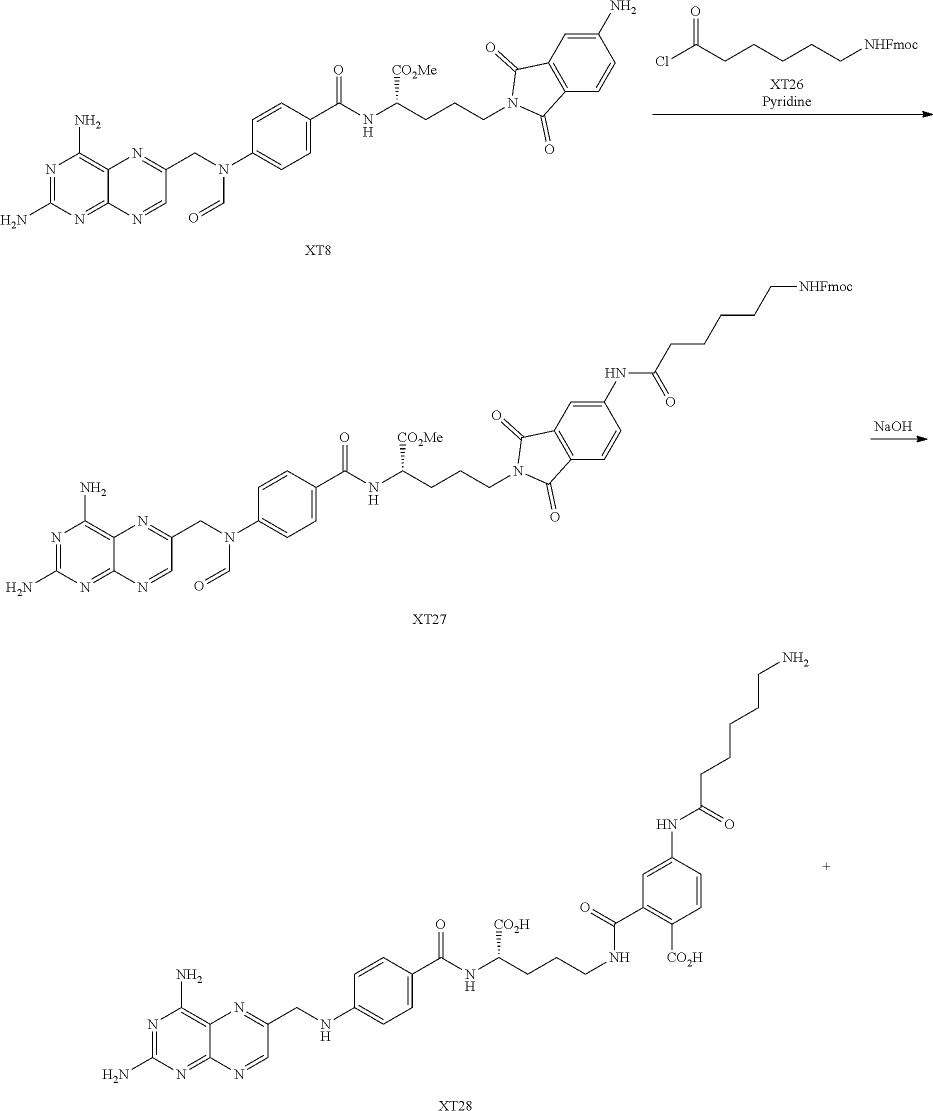

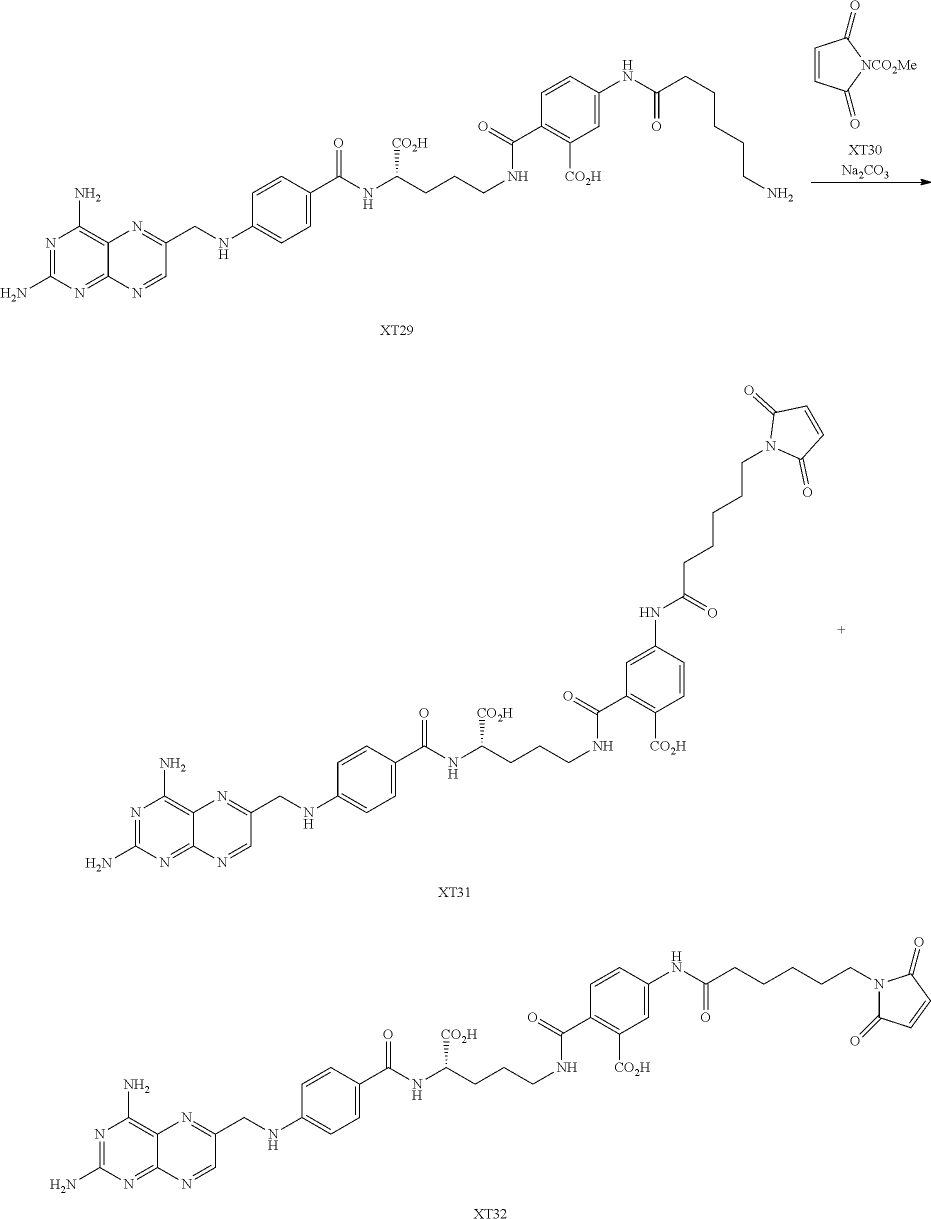

### Methyl (S)-5-(5-(6-((((9H-fluoren-9-yl)methoxy)carbonyl)amino)hexanamido)-1,3-dioxoisoindolin-2-yl)-2-(4-(N-((2,4-diaminopteridin-6-yl)methyl)formamido)benzamido) pentanoate (XT27)

To a suspension of aniline XT8 (100 mg, 0.163 mmol) in pyridine (3 mL) at 0° C., was added acid chloride XT26 (182 mg, 0.490 mmol) (prepared according to Unsworth et al, Angew. Chem. Int. Ed. 2015, 52, 15794-15798) portion-wise (30 mg per portion), and conversion was intermittently checked by UPLC-MS. Once complete, the reaction was quenched with MeOH (2 mL) and concentrated in vacuo. Purification by flash chromatography (silica gel, MeOH:DCM 0:1 to 1:4) afforded amide XT27 (160 mg, quant) together with trace pyridine.

¹H NMR (400 MHz, DMSO-d₆) ppm=10.52 (s, 1H), 8.81 (s, 1H), 8.69 (d, J=7.4 Hz, 1H), 8.19 (s, 1H), 7.98 (br s, 1H), 7.90-7.81 (m, 5H), 7.80-7.73 (m, 1H), 7.67 (d, J=, 7.4 Hz, 2H), 7.59 (d, J=8.3 Hz, 2H), 7.43-7.36 (m, 2H), 7.34-7.25 (m, 3H), 6.94 (br s, 2H), 5.25 (s, 2H), 4.46-4.35 (m, 1H), 4.30-4.25 (m, 2H), 4.21-4.15 (m, 1H), 4.10 (q, J=4.5 Hz, 2H), 3.60 (s, 3H), 3.60-3.52 (m, 2H), 2.99 (q, J=6.1 Hz, 2H), 2.42-2.34 (m, 2H), 1.85-1.56 (m, 6H), 1.48-1.38 (m, 2H), 1.35-1.25 (m, 2H).

¹³C NMR (100 MHz, DMSO-d₆) ppm=173.0, 172.7, 168.2, 168.1, 166.3, 156.6, 145.2, 144.4, 144.1, 141.2, 133.6, 131.1, 129.2, 128.0, 127.7, 127.5, 125.6, 125.6, 124.6, 123.5, 122.1, 120.6, 113.1, 65.6, 52.8, 52.3, 47.2, 46.9, 40.4, 37.4, 36.9, 29.6, 28.3, 26.3, 25.4, 25.1.

MS (ESI⁺) calc. for C₅₀H₅₀N₁₁O₉⁺ [M+H]⁺ 948.38, found 948.45.

### (S)-4-(6-Aminohexanamido)-2-((4-carboxy-4-(4-(((2,4-diaminopteridin-6-yl)methyl)amino) benzamido)-butyl)carbamoyl)benzoic acid (XT28) and (S)-5-(6-aminohexanamido)-2-((4-carboxy-4-(4-(((2,4-diaminopteridin-6-yl)methyl)amino)benzamido)butyl)carbamoyl) benzoic acid (XT29)

The hydrolysis of ester XT27 (145 mg, 0.153 mmol) with NaOH (12 eq) was carried out according to general procedure XXC, with the modification that upon collection of the solid by filtration only ether was used to wash the solid. The crude mixture of diacids XT28 and XT29 (80 mg, 75%, ratio 1:2) thus obtained was carried forward without any further purification.

MS (ESI⁺) calc. for C₃₃H₄₀N₁₁O₇⁺ [M+H]⁺ 702.31, found 702.56.

### (S)-2-((4-Carboxy-4-(4-(((2,4-diaminopteridin-6-yl)methyl)amino)benzamido)butyl) carbamoyl)-4-(6-(2,5-dioxo-2,5-dihydro-1H-pyrrol-1-yl)hexanamido)benzoic acid (XT31) and (S)-2-((4-carboxy-4-(4-(((2,4-diaminopteridin-6-yl)methyl)amino)benzamido)butyl) carbamoyl)-5-(6-(2,5-dioxo-2,5-dihydro-1H-pyrrol-1-yl)hexanamido)benzoic acid (XT32)

To a suspension of diacids XT28 and XT29 (60 mg, 0.086 mmol, 1:2 ratio) in DMF (3 mL) at RT was added aqueous Na₂CO₃ (260 μl, 1 M) followed by N-methoxycarbonylmaleimide XT30 (13.3 mg, 0.086 mmol). More aqueous Na₂CO₃ (0.170 mL, 1 M) was added 3× with 20 min intervals, followed by a final addition of aqueous Na₂CO₃ (0.085 mL, 1 M). After 2 h, the reaction mixture was cooled to 0° C., quenched with aqueous AcOH (2.06 mL, 1 M.) and the resulting suspension concentrated in vacuo. The crude solid was purified by preparative RP-HPLC (water×0.10% TFA/MeCN×0.1% TFA, gradient 20% to 50%). Product fractions were pooled, MeCN was removed by rotary evaporation, and the aqueous solution was lyophilized to yield a mixture of amides XT31 and XT32 (15 mg, 22%) in a 1:2 ratio as a yellow solid.

¹H NMR (400 MHz, DMSO-d₆) ppm=13.41 (br s, 2H), 10.19 (s, 1H), 10.12 (s, 1H), 9.16 (d, J=21.3 Hz, 2H), 8.83 (s, 1H), 8.27-8.12 (m, 2H), 7.94 (d, J=2.0 Hz, 1H), 7.80-7.62 (m, 4H), 7.57 (d, J=1.9 Hz, 1H), 7.34 (d, J=8.4 Hz, 1H), 7.00 (s, 2H), 6.89 (br s, 1H), 6.75 (d, J=8.8 Hz, 2H), 6.55 (br s, 1H), 4.61 (s, 2H), 4.39-4.30 (m, 1H), 3.17 (q, J=5.9 Hz, 2H), 2.35-2.26 (m, 2H), 1.92-1.71 (m, 2H), 1.69-1.45 (m, 6H), 1.31-1.19 (m, 2H).

¹³C NMR (100 MHz, DMSO-d₆) ppm=174.6, 172.2, 172.0, 171.6, 169.2, 168.5, 168.4, 167.3, 166.8, 163.3, 150.9, 150.7, 149.8, 142.6, 141.2, 140.3, 134.9, 133.1, 132.2, 131.3, 129.6, 128.8, 123.8, 122.2, 121.9, 121.0, 119.6, 118.6, 117.9, 111.9, 52.9, 46.1, 37.4, 36.7, 36.6, 28.7, 28.2, 26.6, 26.2, 24.9.

MS (ESI⁺) calc. for C₃₇H₄₀N₁₁O₉⁺ [M+H]⁺ 782.30, found 782.52.

### Preparation of (S)-5-(4-aminobenzamido)-2-(4-(((2,4-diaminopteridin-6-yl)methyl)amino) benzamido)pentanoic acid (XT35)

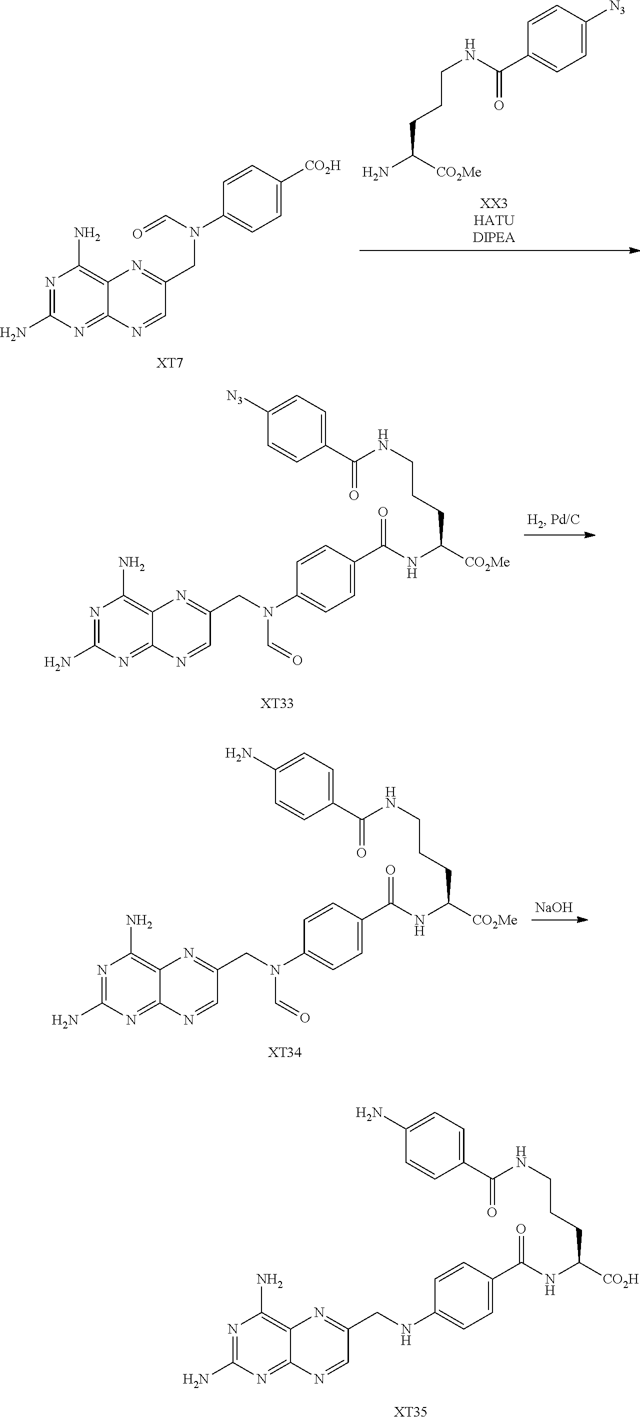

### Methyl (S)-5-(4-azidobenzamido)-2-(4-(N-((2,4-diaminopteridin-6-yl)methyl)formamido) benzamido)-pentanoate (XT33)

Acid XT7 (150 mg, 0.442 mmol) was reacted with amine XX3 (142 mg, 0.486 mmol) according to general procedure XXA. Purification by flash chromatography (silica gel, MeOH:DCM 0:1 to 1:3) afforded ester XT33 (270 mg, quant) as a yellow solid.

MS (ESI⁺) calc. for C28H₂₉N₁₂O₅⁺ [M+H]⁺ 613.24, found 613.50.

### Methyl (S)-5-(4-aminobenzamido)-2-(4-(N-((2,4-diaminopteridin-6-yl)methyl)formamido) benzamido)-pentanoate (XT34)

Reduction of azide XX33 (270 mg, 0.441 mmol) was carried out according to general procedure XXB. Purification by flash chromatography (silica gel, MeOH:DCM 0:1 to 1:3) afforded aniline XT34 (190 mg, 0.324 mmol, 74% yield) as a yellow-grey solid.

¹H NMR (400 MHz, DMSO-d₆) ppm=8.80 (s, 1H), 8.75 (d, J=7.3 Hz, 1H), 8.68 (s, 1H), 7.98 (t, J=5.6 Hz, 1H), 7.90 (d, J=8.6 Hz, 2H), 7.87-7.80 (m, 1H), 7.59 (d, J=8.7 Hz, 2H), 7.55 (d, J=8.6 Hz, 2H), 7.57-7.48 (m, 1H), 6.81 (br s, 2H), 6.52 (d, J=8.6 Hz, 2H), 6.40-4.80 (m, 2H), 5.23 (s, 2H), 4.47-4.38 (m, 1H), 3.62 (s, 3H), 3.22 (q, J=6.0 Hz, 2H), 1.89-1.71 (m, 2H), 1.66-1.48 (m, 2H).

MS (ESI⁺) calc. for C₂₈H₃₁N₁₀O₅⁺ [M+H]⁺ 587.25, found 587.55.

### (S)-5-(4-Aminobenzamido)-2-(4-(((2,4-diaminopteridin-6-yl)methyl)amino)benzamido) pentanoic acid (XT35)

The hydrolysis of ester XT34 (190 mg, 0.324 mmol) with NaOH (6 eq) was carried out according to general procedure XXC, with the following modification. After filtration and washing of the solid, the material was dissolved in a 2% aq. ammonia solution and lyophilized to yield XT35 (143 mg, 0.26 mmol, 81%) as a yellow-orange solid.

¹³C NMR (100 MHz, DMSO-d₆) ppm=174.5, 166.6, 166.4, 163.4, 163.3, 155.7, 151.9, 151.1, 150.1, 146.0, 129.3, 129.1, 122.2, 121.9, 121.3, 113.0, 111.9, 53.3, 46.1, 40.9, 29.4, 26.7.

MS (ESI⁺) calc. for C26H₂₉N₁₀O₄⁺ [M+H]⁺ 545.24, found 545.23.

### Preparation of 2-(((S)-4-carboxy-4-(4-(2-(2,4-diaminopteridin-6-yl)ethyl)benzamido)butyl) carbamoyl)-5-((S)-2-((S)-2-(6-(2,5-dioxo-2,5-dihydro-1H-pyrrol-1-yl)hexanamido)-3-methylbutanamido)propanamido)benzoic acid (XT41)

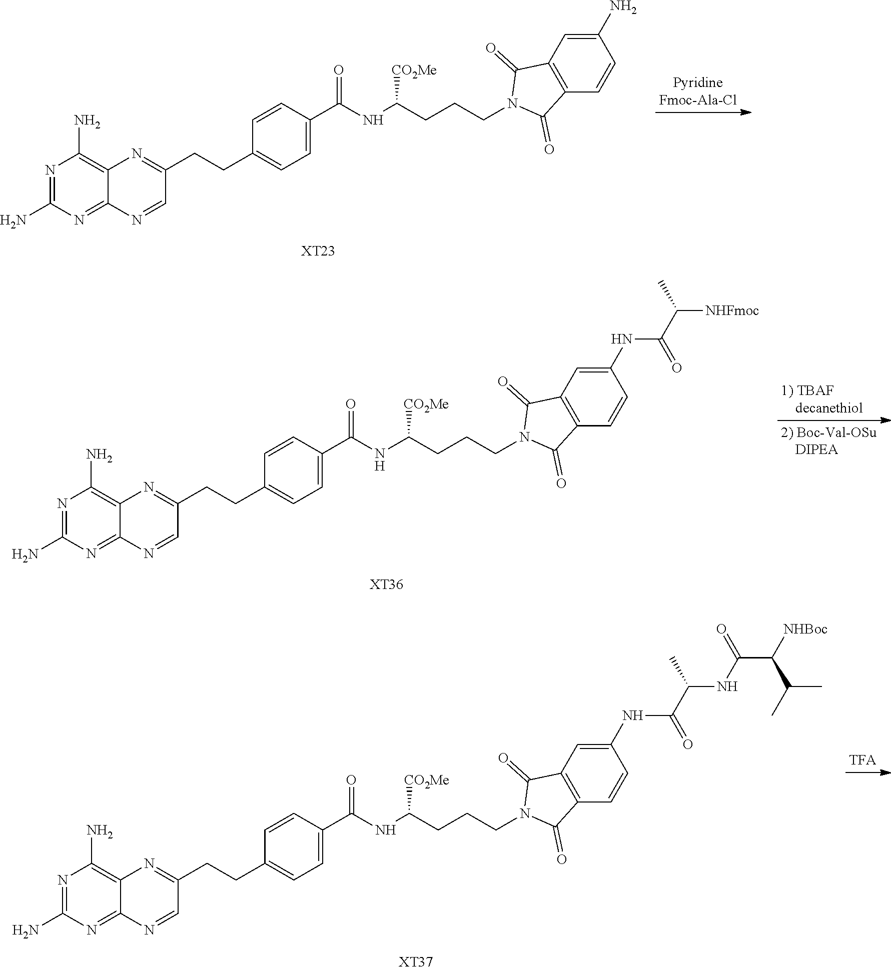

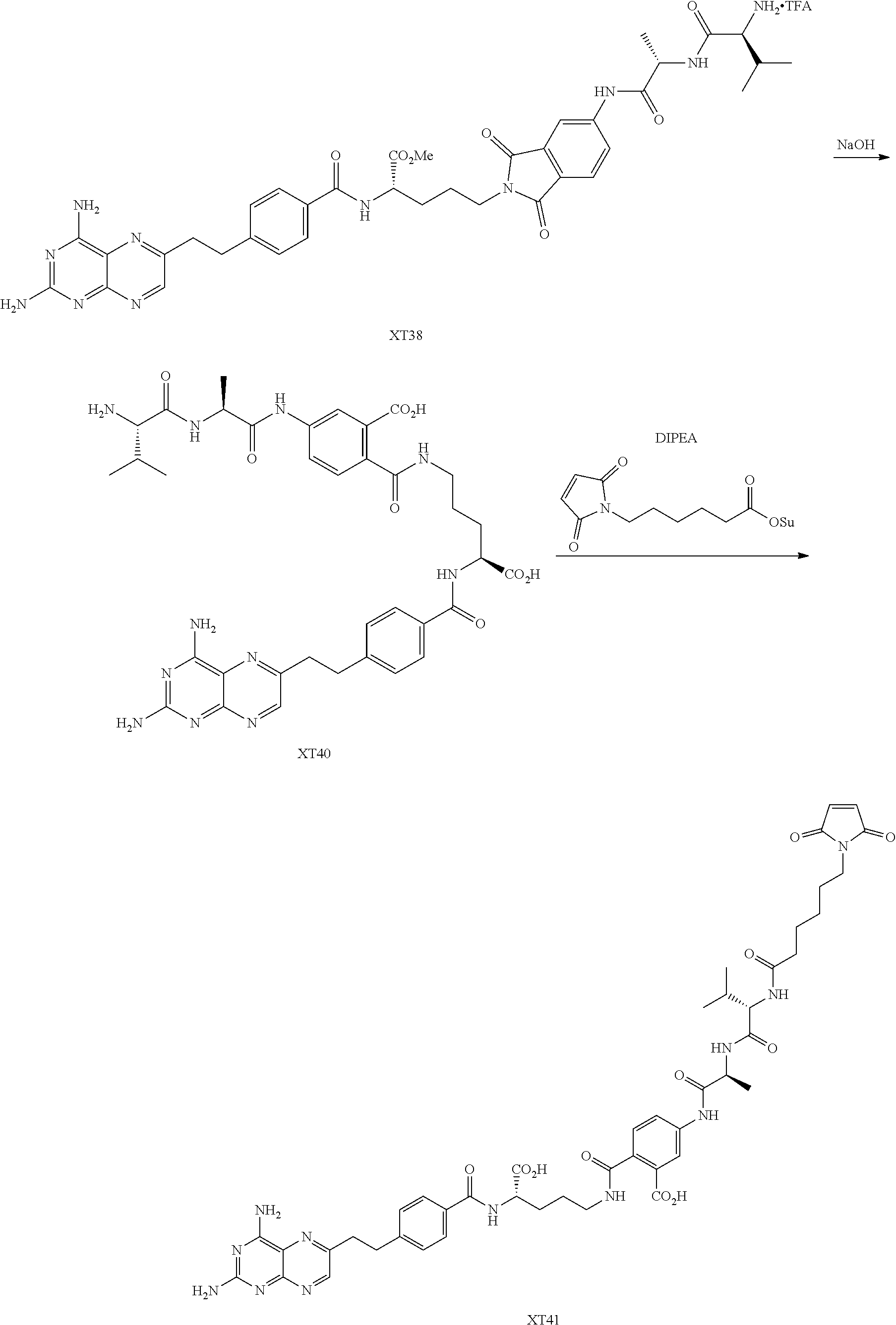

### Methyl (S)-5-(5-((S)-2-((((9H-fluoren-9-yl)methoxy)carbonyl)amino)propanamido)-1,3-dioxoisoindolin-2-yl)-2-(4-(2-(2,4-diaminopteridin-6-yl)ethyl)benzamido)pentanoate (XT36)

Aniline XT23 (280 mg, 0.480 mmol) was reacted with Fmoc-Ala-Cl (396 mg, 1.20 mmol) according to the procedure for XT11. Purification by flash chromatography (silica gel, MeOH:DCM 0:1 to 1:4) afforded amide XT36 (350 mg, 83%) as a yellow solid.

MS (ESI⁺) calc. for C₄₇H₄₅N₁₀O₈⁺ [M+H]⁺ 877.34, found 877.44.

### Methyl (S)-5-(5-((S)-2-((S)-2-((tert-butoxycarbonyl)amino)-3-methylbutanamido) propanamido)-1,3-dioxoisoindolin-2-yl)-2-(4-(2-(2,4-diaminopteridin-6-yl)ethyl)benzamido) pentanoate (XT37)

Fmoc-protected amine XT36 (350 mg, 0.399 mmol) was deprotected with TBAF·3H₂O and decanethiol, and subsequently reacted with Boc-Val-OSu and DIPEA according to the procedure for XT12. Purification by flash chromatography (silica gel, MeOH:DCM 0:1 to 1:4) afforded dipeptide XT37 (quant), contaminated with tetrabutylamine salts.

MS (ESI⁺) calc. for C₄₂H₅₂N₁₁O₉⁺ [M+H]⁺ 854.39, found 854.49.

### Methyl (S)-5-(5-((S)-2-((S)-2-amino-3-methylbutanamido)propanamido)-1,3-dioxoisoindolin-2-yl)-2-(4-(2-(2,4-diaminopteridin-6-yl)ethyl)benzamido)pentanoate (XT38)

Dipeptide XT37 (340 mg, 0.400 mmol) was deprotected according to the procedure for XT13. After concentration, TFA-salt XT38 was directly used in the next step.

MS (ESI⁺) calc. for C₃₇H₄₄N₁₁O₇⁺ [M+H]⁺ 754.34, found 754.47.

### 5-((S)-2-((S)-2-Amino-3-methylbutanamido)propanamido)-2-(((S)-4-carboxy-4-(4-(2-(2,4-diaminopteri-din-6-yl)ethyl)benzamido)butyl)carbamoyl)benzoic acid (XT40)

The hydrolysis of formamide XT38 (300 mg, 0.400 mmol) was carried out according to general procedure XXC, with the following modifications. Formamide XT38 was first reacted with NaOH (12 eq) at 0° C. for 1 h, and then after addition of a second portion of NaOH (12 eq), for 6 h at RT. Washing of the solid with MeOH was replaced with washing with water (3 mL). Ring-opening of the phthalimide proceeded with modest selectivity, favoring the desired regioisomer XT40 (˜1:2 ratio). The material was purified by preparative RP-HPLC (water×0.1% TFA/MeCN×0.1% TFA, gradient 5% to 25%), to afford XT40 (50 mg, 16%, 3 steps) as a white solid.

¹H NMR (400 MHz, DMSO-d₆) ppm=13-83-11.98 (m, 2H), 10.40 (s, 1H), 9.06 (br s, 1H), 8.96 (br s, 1H), 8.76 (d, J=6.7 Hz, 1H), 8.72 (s, 1H), 8.53 (d, J=7.7 Hz, 1H), 8.26 (t, J=5.6 Hz, 1H), 8.11 (br s, 3H), 7.98 (d, J=2.1 Hz, 1H), 7.83 (d, J=8.2 Hz, 2H), 7.72 (dd, J=8.4, 2.1 Hz, 1H), 7.39 (d, J=8.4 Hz, 1H), 7.37 (d, J=8.2 Hz, 2H), 6.58 (br s, 1H), 4.49 (p, J=7.0 Hz, 1H), 4.43-4.28 (m, 1H), 3.63 (d, J=5.1 Hz, 1H), 3.27-3.14 (m, 6H), 2.09 (sextet, J=6.6 Hz, 1H), 1.96-1.86 (m, 1H), 1.86-1.73 (m, 1H), 1.70-1.51 (m, 2H), 1.37 (d, J=7.1 Hz, 3H), 0.96 (dd, J=6.7, 4.8 Hz, 6H).

MS (ESI⁺) calc. for C₃₆H₄₄N₁₁O₈⁺ [M+H]⁺ 758.34, found 758.61.

### 2-(((S)-4-Carboxy-4-(4-(((2,4-diaminopteridin-6-yl)methyl)amino)benzamido)butyl) carbamoyl)-5-((S)-2-((S)-2-(6-(2,5-dioxo-2,5-dihydro-1H-pyrrol-1-yl)hexanamido)-3-methyl-butanamido)propanamido)benzoic acid (XT41)

Amine XT40 (45 mg, 0.059 mmol) was reacted with 6-maleimidohexanoic acid N-hydroxylsuccinimide ester (28 mg, 0.089 mmol) and DIPEA (0.062 mL, 0.36 mmol) according to the procedure for XT17. Purification by preparative RP-HPLC (water×0.1% TFA/MeCN×0.1% TFA, gradient 20% to 50%) gave XT41 (30 mg, 53%) as a white solid.

¹H NMR (400 MHz, DMSO-d₆) ppm=13.25-12.33 (m, 2H), 10.15 (s, 1H), 9.25 (s, 1H), 9.16 (s, 1H), 8.75 (s, 1H), 8.57 (br s, 1H), 8.52 (d, J=7.7 Hz, 1H), 8.24 (t, J=5.6 Hz, 1H), 8.21 (d, J=6.7 Hz, 1H), 7.97 (d, J=1.9 Hz, 1H), 7.85-7.78 (m, 3H), 7.74 (dd, J=8.4, 1.9 Hz, 1H), 7.72-7.54 (m, 1H), 7.38 (d, J=8.2 Hz, 1H), 7.36 (d, J=8.2 Hz, 2H), 7.00 (s, 2H), 4.42-4.31 (m, 2H), 4.17 (dd, J=8.0, 7.4 Hz, 1H), 3.32-3.14 (m, 8H), 2.22-2.07 (m, 2H), 2.01-1.73 (m, 3H), 1.69-1.41 (m, 6H), 1.31 (d, J=7.1 Hz, 3H), 1.24-1.13 (m, 2H), 0.87 (d, J=6.6 Hz, 3H), 0.82 (d, J=6.8 Hz, 3H).

MS (ESI⁺) calc. for C₄₅H₅₅N₁₃O₁₁⁺ [M+H]⁺ 951.41, found 951.91.

### Preparation of (S)-2-(4-(2-(2,4-diaminopteridin-6-yl)ethyl)benzamido)-5-(4-((S)-2-((S)-2-(6-(2,5-dioxo-2,5-dihydro-1H-pyrrol-1-yl)hexanamido)-3-methylbutanamido)propanamido) benzamido)pentanoic acid (XT46)

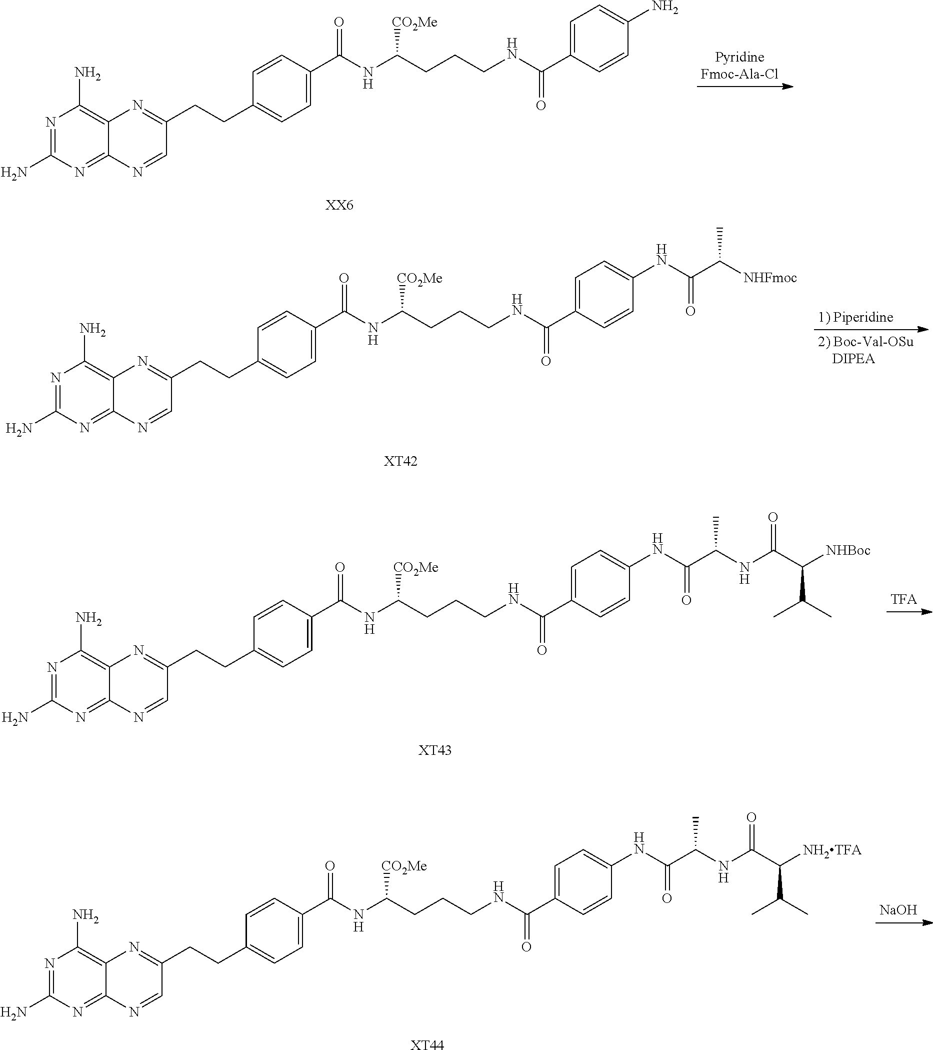

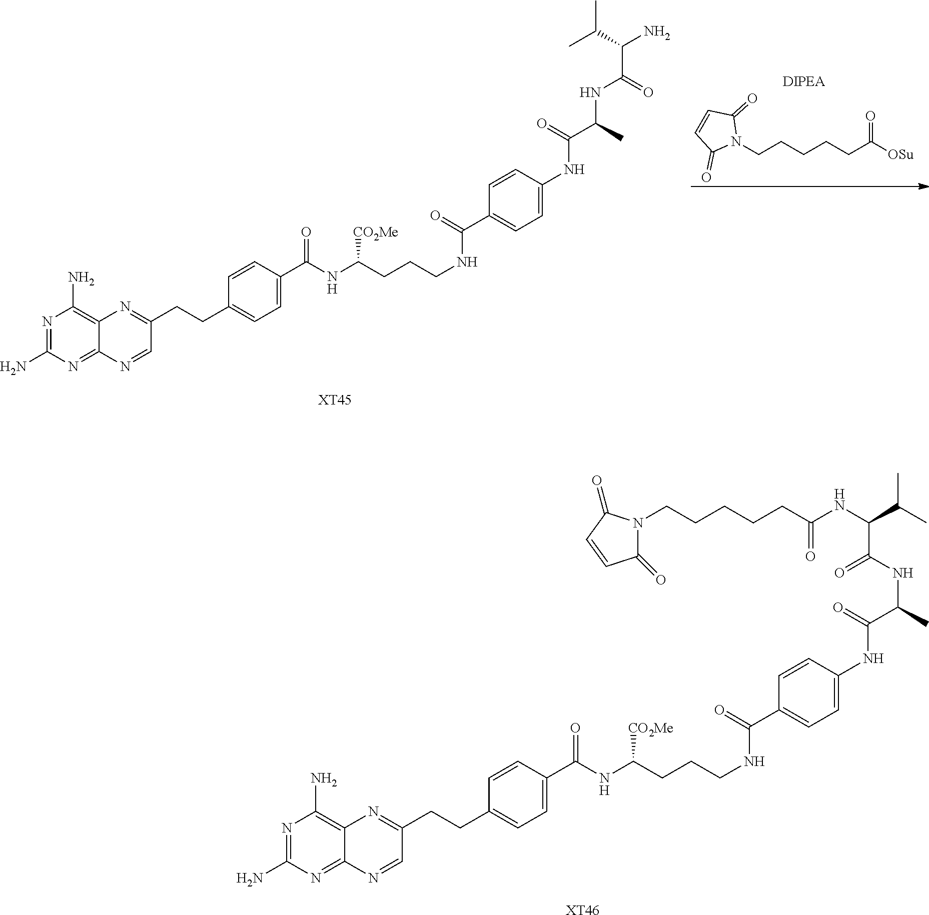

### Methyl (S)-5-(4-((S)-2-((((9H-fluoren-9-yl)methoxy)carbonyl)amino)propanamido) benzamido)-2-(4-(2-(2,4-diaminopteridin-6-yl)ethyl)benzamido)pentanoate (XT42)

Aniline XX6 (150 mg, 0.269 mmol) was reacted with Fmoc-Ala-Cl (310 mg, 0.942 mmol) according to the procedure for XT11. Purification by flash chromatography (silica gel, MeOH:DCM 0:1 to 1:4) afforded amide XT42 (180 mg, 79%) as an off-white solid.

¹H NMR (400 MHz, DMSO-d₆) ppm=10.25 (s, 1H), 9.07 (br s, 1H), 8.98 (br s, 1H), 8.72 (s, 1H), 8.70 (d, J=7.5 Hz, 1H), 8.58 (d, J=4.0 Hz, 1H), 8.38 (t, J=5.1 Hz, 1H), 7.90 (d, J=7.4 Hz, 2H), 7.86-7.77 (m, 4H), 7.76-7.70 (m, 3H), 7.67 (d, J=8.6 Hz, 2H), 7.59 (br s, 1H), 7.45-7.28 (m, 7H), 7.10 (br s, 1H), 4.49-4.41 (m, 1H), 4.31-4.25 (m, 2H), 4.25-4.16 (m, 2H), 3.63 (s, 3H), 3.28-3.16 (m, 4H), 1.92-1.76 (m, 2H), 1.72-1.52 (m, 2H), 1.32 (d, J=7.1 Hz, 3H).

MS (ESI⁺) calc. for C₄₆H₄₇N₁₀O₇⁺ [M+H]⁺ 851.36, found 851.82.

### Methyl (S)-5-(4-((S)-2-((S)-2-((tert-butoxycarbonyl)amino)-3-methylbutanamido) propanamido)benzamido)-2-(4-(2-(2,4-diaminopteridin-6-yl)ethyl)benzamido)pentanoate (XT43)

To a solution of XT42 (180 mg, 0.212 mmol) in DMF (4 mL) was added piperidine (0.419 mL, 4.23 mmol) and the resulting mixture was stirred for 15 min at RT. The reaction mixture was concentrated and coevaporated with toluene. Ether (50 mL) was added and the resulting suspension was stirred at RT for 10 min. After filtration, the solids were collected and the crude amine was taken up in DMF (4 mL). Next, Boc-Val-OSu (100 mg, 0.317 mmol) and DIPEA (0.074 mL, 0.42 mmol) were added and the resulting mixture was stirred for 4 h. The reaction mixture was subsequently concentrated and the crude product was purified by flash chromatography (silica gel, MeOH:DCM 0:1 to 1:4) to give dipeptide XT43 (170 mg, 97%).

¹H NMR (400 MHz, DMSO-d₆) ppm=10.45 (s, 1H), 10.21 (s, 1H), 8.85 (br s, 1H), 8.68 (d, J=7.4 Hz, 1H), 8.56 (s, 1H), 8.36 (t, J=5.5 Hz, 1H), 8.12 (d, J=6.8 Hz, 1H), 7.79 (d, J=8.5 Hz, 4H), 7.67 (br s, 1H), 7.65 (d, J=8.7 Hz, 2H), 7.34 (d, J=8.1 Hz, 2H), 6.71 (d, J=8.8 Hz, 1H), 6.60 (br s, 2H), 4.49-4.38 (m, 2H), 3.83 (dd, J=8.0, 7.5 Hz, 1H), 3.63 (s, 3H), 3.29-3.23 (m, 2H), 3.19-3.07 (m, 4H), 2.02-1.90 (m, 1H), 1.90-1.74 (m, 2H), 1.70-1.50 (m, 2H), 1.38 (s, 9H), 1.31 (d, J=7.1 Hz, 3H), 0.87 (d, J=7.0 Hz, 3H), 0.82 (d, J=6.5 Hz, 3H).

MS (ESI⁺) calc. for C₄₁H₅₄N₁₁O₈⁺ [M+H]⁺ 828.42, found 828.81.

### Methyl (S)-5-(4-((S)-2-((S)-2-amino-3-methylbutanamido)propanamido)benzamido)-2-(4-(2-(2,4-diaminopteridin-6-yl)ethyl)benzamido)pentanoate (XT44)

Dipeptide XT43 (340 mg, 0.400 mmol) was deprotected according to the procedure for XT13. After concentration, TFA-salt XT44 was directly used in the next step.

MS (ESI⁺) calc. for C₃₆H₄₆N₁₁O₆⁺ [M+H]⁺ 728.36, found 728.66.

### (S)-5-(4-((S)-2-((S)-2-Amino-3-methylbutanamido)propanamido)benzamido)-2-(4-(2-(2,4-diaminopteridin-6-yl)ethyl)benzamido)pentanoic acid (XT45)

The hydrolysis of ester XT44 (145 mg, 0.153 mmol) with NaOH (12 eq) was carried out according to general procedure XXC, with the modification that upon treatment with aq.

AcOH (1 M) the product did not solidify and therefore the resulting solution was lyophilized after evaporation of MeOH. The resulting cake was stirred with DMF (5 mL) and filtered, the obtained DMF solution containing XT45 was used directly in the next step.

MS (ESI⁺) calc. for C₃₅H₄₄N₁₁O⁺ [M+H]⁺ calc. 714.35, found 714.71.

### (S)-2-(4-(2-(2,4-Diaminopteridin-6-yl)ethyl)benzamido)-5-(4-((S)-2-((S)-2-(6-(2,5-dioxo-2,5-dihydro-1H-pyrrol-1-yl)hexanamido)-3-methylbutanamido)propanamido) benzamido) pentanoic acid (XT46)

Amine XT45 was reacted with 6-maleimidohexanoic acid N-hydroxylsuccinimide ester (49 mg, 0.16 mmol) and DIPEA (0.168 mL, 0.960 mmol) according to the procedure for XT17. Purification by preparative RP-HPLC (water×0.1% TFA/MeCN×0.1% TFA, gradient 20% to 50%) gave XT46 (67 mg, 46%, 3 steps) as a white solid.

¹H NMR (400 MHz, DMSO-d₆) ppm=13.13 (br s, 1H), 10.11 (s, 1H), 9.25 (s, 1H), 9.16 (s, 1H), 8.75 (s, 1H), 8.57 (br s, 1H), 8.55 (d, J=7.7 Hz, 1H), 8.36 (t, J=5.6 Hz, 1H), 8.20 (d, J=6.7 Hz, 1H), 7.92-7.75 (m, 6H), 7.66 (d, J=8.7 Hz, 2H), 7.36 (d, J=8.2 Hz, 2H), 6.99 (s, 2H), 4.44-4.33 (m, 2H), 4.17 (dd, J=7.9, 7.5 Hz, 1H), 3.37 (t, J=6.9 Hz, 2H), 3.31-3.16 (m, 6H), 2.23-2.06 (m, 2H), 2.01-1.73 (m, 3H), 1.71-1.54 (m, 2H), 1.53-1.41 (m, 4H), 1.32 (d, J=7.1 Hz, 3H), 1.23-1.13 (m, 2H), 0.86 (d, J=6.6 Hz, 3H), 0.82 (d, J=6.8 Hz, 3H).

MS (ESI⁺) calc. for C₄₅H₅₅N₁₂O₉⁺ [M+H]⁺ 907.42, found 907.84.

### Preparation of methyl (S)-5-amino-2-(4-(N-((2,4-diaminopteridin-6-yl)methyl) formamido)-benzamido)pentanoate hydrochloride (XT48)

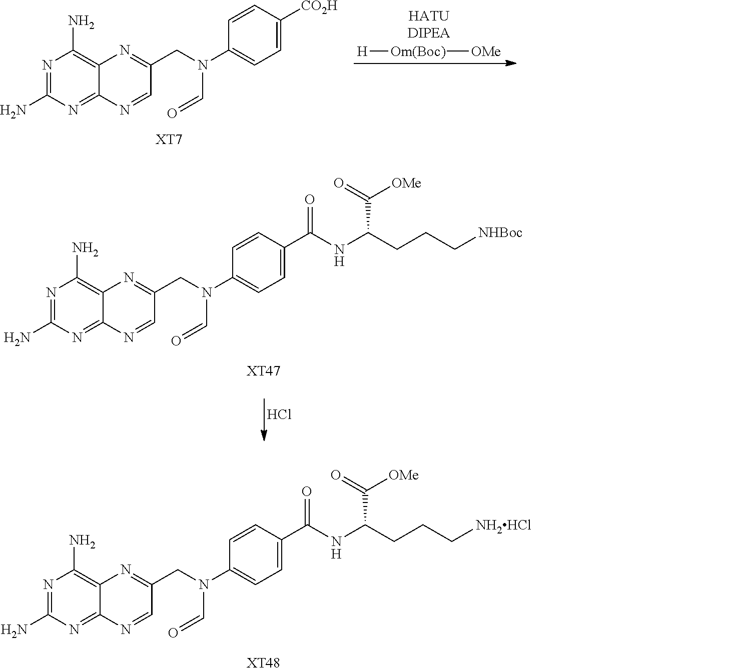

### Methyl (S)-5-((tert-Butoxycarbonyl)amino)-2-(4-(N-((2,4-diaminopteridin-6-yl)methyl) formamido)-benzamido)pentanoate (XT47)

Acid XT7 (1.30 g, 3.83 mmol) was reacted with H-Orn(Boc)-OMe (1.30 g, 4.60 mmol) according to general procedure XXA. Purification by flash chromatography (silica gel, MeOH:DCM 0:1 to 1:4) afforded ester XT47 (1.80 g, 83%) as an orange/red solid foam.

¹H NMR (400 MHz, DMSO-d₆) ppm=8.81 (s, 1H), 8.72-8.67 (m, 1H), 8.70 (s, 1H), 7.99 (br s, 1H), 7.90 (d, J=8.5 Hz, 2H), 7.68 (br s, 1H), 7.60 (d, J=8.7 Hz, 2H), 6.96 (br s, 2H), 6.79 (t, J=5.2 Hz, 1H), 5.25 (s, 2H), 4.44-4.35 (m, 1H), 3.63 (s, 3H), 2.99-2.88 (m, 2H), 1.86-1.76 (m, 2H), 1.55-1.38 (m, 2H), 1.36 (s, 9H).

MS (ESI⁺) calc. for C₂₆H₃₄N₉O₆⁺ [M+H]⁺ 568.26, found 568.56.

### Methyl (S)-5-amino-2-(4-(N-((2,4-diaminopteridin-6-yl)methyl)formamido)benzamido) pentanoate hydrochloride (XT48)

Ester XT47 (1.60 g, 2.82 mmol) was heated in dioxane (16 mL) until dissolved. After cooling to RT, the solution was then dropwise added to HCl in dioxane (4 M, 30 mL) under vigorous stirring. The resulting yellow suspension was stirred for 2 h. The reaction mixture was then filtered and the residue was washed with ether (50 mL), taken up in water (30 mL) and lyophilized to yield XT48 (1.42 g, 100%) as a yellow solid.

¹H NMR (400 MHz, DMSO-d₆) ppm=13.21 (br s, 1H), 9.29 (s, 1H), 8.98 (s, 1H), 8.87 (d, J=7.4 Hz, 1H), 8.83 (d, J=5.2 Hz, 1H), 8.68 (br s, 1H), 8.01 (br s, 4H), 7.95 (d, J=8.6 Hz, 2H), 7.61 (d, J=8.6 Hz, 2H), 5.33 (s, 2H), 4.46-4.38 (m, 1H), 3.63 (s, 3H), 2.85-2.71 (m, 2H), 1.95-1.79 (m, 2H), 1.75-1.57 (m, 2H).

MS (ESI⁺) calc. for C₂₁H₂₆N₉O₄⁺ [M+H]⁺ 468.21, found 468.52.

### Preparation of 5-((S)-5-benzyl-18-(2,5-dioxo-2,5-dihydro-1H-pyrrol-1-yl)-4,7,10,13-tetraoxo-3,6,9,12-tetraazaoctadecanamido)-2-(((S)-4-carboxy-4-(4-(((2,4-diaminopteridin-6-yl)methyl)amino)benzamido)butyl)carbamoyl)benzoic acid (XT54)

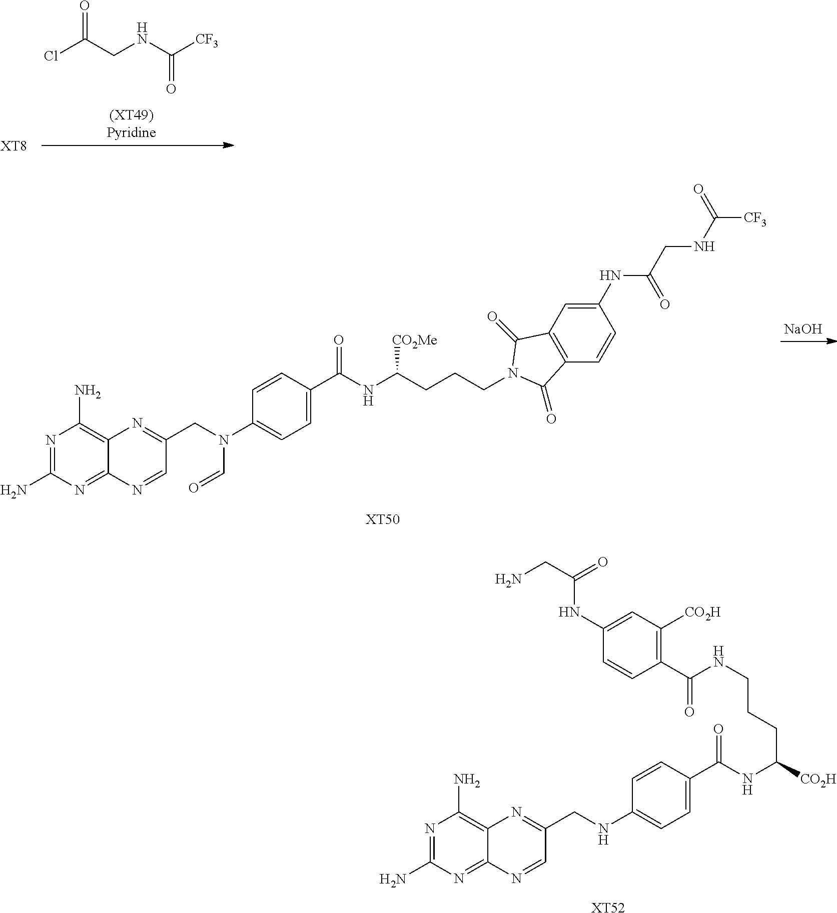

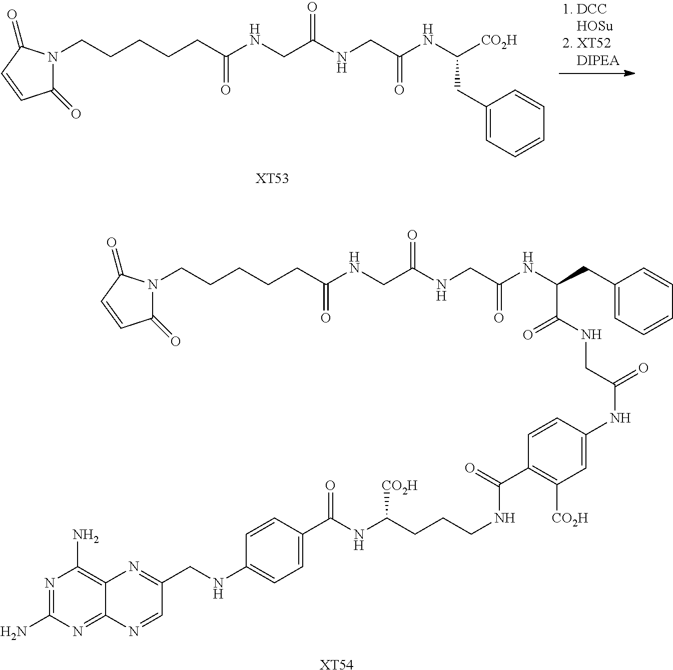

### (2,2,2-Trifluoroacetyl)glycinoyl chloride (XT49)

To a solution of glycine (2.50 g, 33.3 mmol) in MeOH (33 mL) and Et₃N (6.96 mL, 50.0 mmol) was added ethyl trifluoroacetate (5.17 mL, 43.3 mmol). The resulting mixture was stirred for 18 h at RT. The solution was concentrated and the residue was taken up in EtOAc (75 mL), the organic solution was washed with aqueous HCl (1 M, 150 mL), the aq. layer was back-extracted with EtOAc (2×75 mL). The combined organic extracts were washed with brine (50 mL), dried over MgSO₄, filtered and the filtrate concentrated to yield (2,2,2-trifluoroacetyl)glycine (5.3 g, 93%) as a white solid. A portion of the product (1.00 g, 5.85 mmol) was suspended in DCM (30 mL), cooled to 0° C. and oxalyl chloride (1.54 mL, 17.54 mmol) was added dropwise, followed by 2 drops of DMF. The resulting mixture was stirred for 1 h at RT and was subsequently concentrated and coevaporated with toluene (2×) to yield XT49 (quant). Quenching of the product with MeOH afforded the corresponding methyl ester.

MS (ESI⁺) calc. for C₅H₇F₃NO₃⁺ [M+H]⁺ 186.04, found 186.36.

### Methyl (S)-2-(4-(N-((2,4-diaminopteridin-6-yl)methyl)formamido)benzamido)-5-(1,3-dioxo-5-(2-(2,2,2-trifluoroacetamido)acetamido)isoindolin-2-yl)pentanoate (XT50)

A suspension of XT8 (90.0 mg, 0.147 mmol) in pyridine (2.5 mL) was cooled to 0° C. and XT49 (167 mg, 0.88 mmol) was added in 6 portions over a 1 h period. Once UPLC-MS indicated full conversion, the reaction was quenched with MeOH (3 mL) and concentrated in vacuo. The crude residue was purified by flash chromatography (silica gel, MeOH:DCM 0:1 to 1:3) to afford XT50 (118 mg, quant) as a yellow solid.

MS (ESI⁺) calc. for C₃₃H₃₁F₃N₁₁O₈⁺ [M+H]⁺ 766.23, found 766.61.

### (S)-5-(2-aminoacetamido)-2-((4-carboxy-4-(4-(((2,4-diaminopteridin-6-yl)methyl)amino) benzamido)butyl)carbamoyl)benzoic acid (XT52)

The hydrolysis of formamide XT50 (107 mg, 0.14 mmol) was carried out according to general procedure XXC, with the following modification. Formamide XT50 was first reacted with NaOH (12 eq) at 0° C. for 1 h, and then after addition of a second portion of NaOH (12 eq) for 3 h at RT. Washing of the solid with MeOH was replaced with washing with water (3 mL). Ring-opening of the phthalimide proceeded with modest selectivity, favoring the desired regioisomer XT52 (˜1:2 ratio). The crude was purified by preparative RP-HPLC (water×0.1% TFA/MeCN×0.1% TFA, gradient 5% to 35%), to afford diacid XT52 (36 mg, 40%, 2 steps) as a yellow solid.

¹H NMR (400 MHz, DMSO-d₆) ppm=13.79-12.05 (m, 2H), 10.67 (s, 1H), 9.17 (br s, 1H), 9.13 (br s, 1H), 8.83 (s, 1H), 8.37 (br s, 1H), 8.28 (t, J=5.6 Hz, 1H), 8.19-8.05 (m, 3H), 7.96 (d, J=1.9 Hz, 1H), 7.77-7.69 (m, 4H), 7.42 (d, J=8.4 Hz, 1H), 6.63 (br s, 1H), 6.75 (d, J=8.7, 2H), 6.54 (br s, 1H), 4.61 (s, 2H), 4.39-4.30 (m, 1H), 3.81 (s, 2H), 3.19 (q, J=6.2 Hz, 2H), 1.94-1.83 (m, 1H), 1.83-1.71 (m, 1H), 1.69-1.49 (m, 2H).

MS (ESI⁺) calc. for C₂₉H₃₂N₁₁O₇⁺ [M+H]⁺ 646.25, found 646.67.

### 5-((S)-5-Benzyl-18-(2,5-dioxo-2,5-dihydro-JH-pyrrol-1-yl)-4,7,10,13-tetraoxo-3,6,9,12-tetraazaoctadecanamido)-2-(((S)-4-carboxy-4-(4-(((2,4-diaminopteridin-6-yl)methyl) amino)benzamido)butyl)carbamoyl)benzoic acid (XT54)

To a suspension of tripeptide XT53 (synthesized as described in EP 2907824) (105 mg, 0.22 mmol) in THF (4 mL) were added N,N′-dicyclohexylcarbodiimide (DCC; 46 mg, 0.22 mmol) and N-hydroxysuccinimide (26 mg, 0.22 mmol) and the resulting suspension was stirred for 18 h. The obtained suspension was filtered and a portion of the filtrate (1.0 mL) was added to a solution of diacid XT52 (32 mg, 0.05 mmol) in DMF (0.5 mL). DIPEA (0.052 mL, 0.30 mmol) was added and the resulting solution was stirred for 30 min at RT. After concentration, the crude solid was purified by preparative RP-HPLC (water×0.1% TFA/MeCN×0.1% TFA, gradient 20% to 50%). Product fractions were pooled, MeCN was removed by rotary evaporation, and the aqueous solution was lyophilized to yield XT54 (22 mg, 40%) as a yellow solid.

¹H NMR (400 MHz, DMSO-d₆) ppm=13.28-12.29 (m, 2H), 10.12 (s, 1H), 9.20 (br s, 1H), 9.15 (br s, 1H), 8.82 (s, 1H), 8.40 (t, J=5.7 Hz, 1H), 8.25 (t, J=5.5 Hz, 1H), 8.18-8.12 (m, 2H), 8.07 (t, J=5.7 Hz, 1H), 8.02 (t, J=5.6 Hz, 1H), 8.00 (d, J=2.0 Hz, 1H), 7.77-7.71 (m, 3H), 7.39 (d, J=8.4 Hz, 1H), 7.28-7.15 (m, 6H), 6.99 (s, 2H), 6.89 (br s, 1H), 6.74 (d, J=8.7 Hz, 2H), 6.53 (br s, 1H), 4.61 (d, J=3.2 Hz, 2H), 4.46-4.48 (m, 1H), 4.39-4.29 (m, 1H), 3.90 (t, J=4.6 Hz, 2H), 3.80-3.55 (m, 6H), 3.19 (q, J=5.9 Hz, 2H), 3.08 (dd, J=13.9, 4.5 Hz, 1H), 2.83 (dd, J=13.8, 9.8 Hz, 1H), 2.10 (t, J=7.5 Hz, 2H), 1.94-1.70 (m, 2H), 1.69-1.39 (m, 6H), 1.26-1.14 (m, 2H).

MS (ESI⁺) calc. for C₅₂H₅₈N₁₅O₁₃⁺ [M+H]⁺ 1100.43, found 1100.85.

### Preparation of 2-(((S)-5-carboxy-5-(4-(((2,4-diaminopteridin-6-yl)methyl)amino) benzamido)-pentyl)amino)-5-((S)-2-((S)-2-(6-(2,5-dioxo-2,5-dihydro-1H-pyrrol-1-yl) hexanamido)-3-methylbutanamido)propanamido)benzoic acid (XT63)

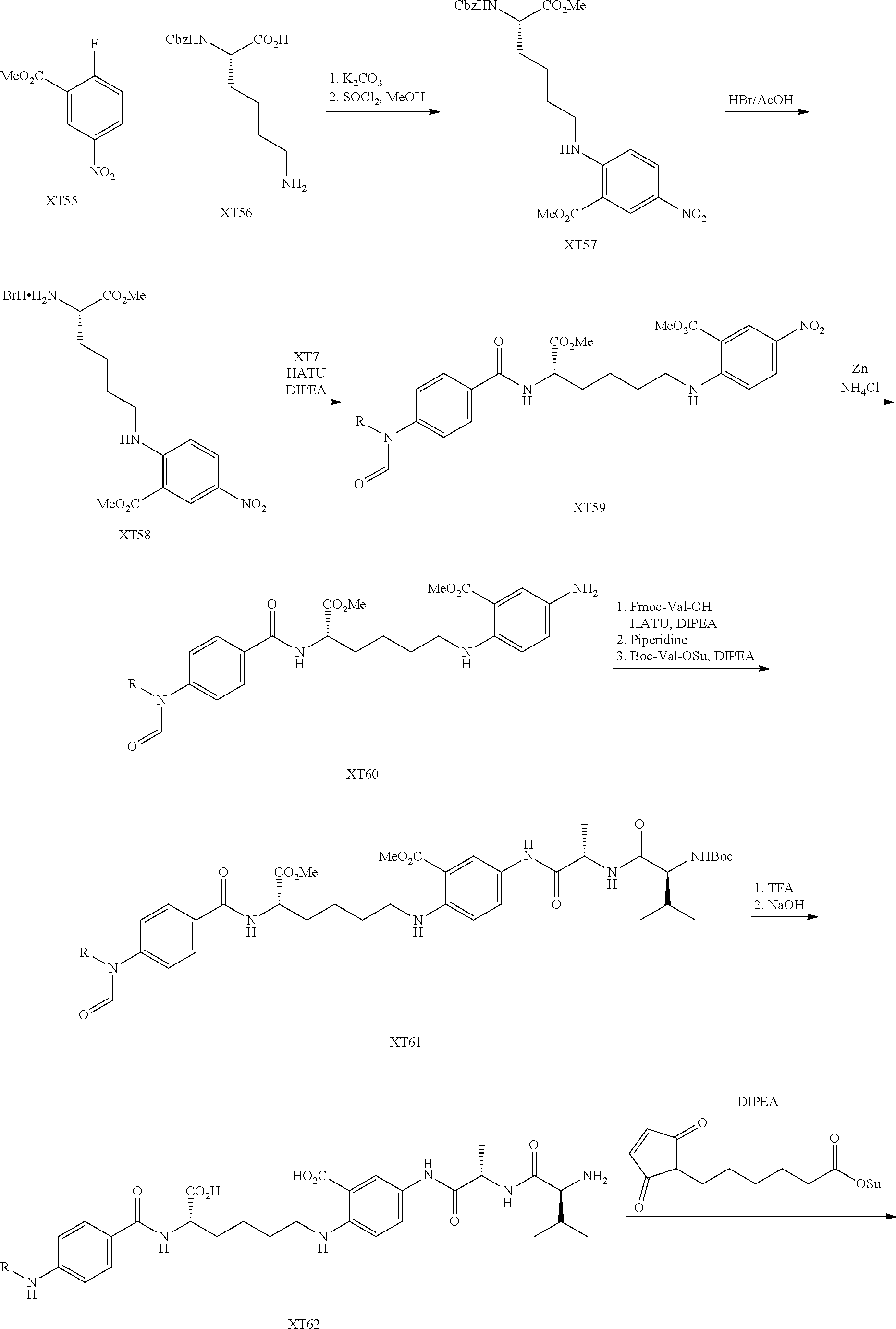

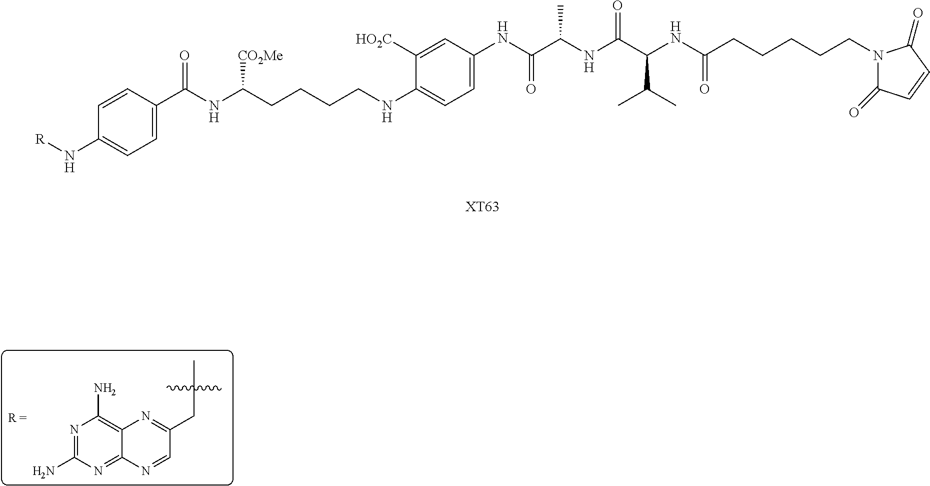

### Methyl (S)-2-((5-(((benzyloxy)carbonyl)amino)-6-methoxy-6-oxohexyl)amino)-5-nitrobenzoate (XT57)

A suspension of XT55 (1.42 g, 7.13 mmol), XT56 (2.0 g, 7.13 mmol) and K₂CO₃ (1.48 g, 10.7 mmol) in MeCN (15 mL) was heated to 90° C. in a sealed tube for 2 h. Concentrated HCl (0.89 mL, 11 mmol) was added at RT and the crude reaction mixture was concentrated. Next, the carboxylic acid intermediate was taken up in MeOH (100 mL) cooled to 0° C. and thionyl chloride (5.8 mL, 79 mmol) was added. The resulting solution was stirred for 1 h at 0° C., and was then gradually warmed to RT over 1 h. Upon completion, the reaction mixture was filtered over Celite and the filtrate was concentrated in vacuo. The crude product was purified by flash chromatography (silica gel, MeOH:DCM 0:1 to 1:9) to give aniline XT57 (2.8 g, 83%) as a yellow foam.

¹H NMR (400 MHz, DMSO-d₆) ppm=8.64 (d, J=2.8 Hz, 1H), 8.54 (t, J=5.4 Hz, 1H), 8.19 (dd, J=9.5, 1.7 Hz, 1H), 7.75 (d, J=7.8 hz, 1H), 7.42-7.25 (m, 5H), 6.93 (d, J=9.6 Hz, 1H), 5.03 (s, 2H), 4.08-4.00 (m, 1H), 3.86 (s, 3H), 3.63 (s, 3H), 3.34 (q, J=6.3 Hz, 2H), 1.79-1.52 (m, 4H), 1.50-1.34 (m, 2H).

MS (ESI⁺) calc. for C₂₃H₂₈N₃O₈⁺ [M+H]⁺ 474.19, found 474.31.

### Methyl (S)-2-((5-amino-6-methoxy-6-oxohexyl)amino)-5-nitrobenzoate hydrobromide (XT58)

A solution of XT57 (750 mg, 1.58 mmol) in DCM (8 mL) was cooled to 0° C. Ice-cold HBr in AcOH (33%, 10 mL) was added and the resulting solution was stirred for 2 h at 0° C. The reaction mixture was subsequently concentrated in vacuo and coevaporated with toluene to give amine XT58 (660 mg, 99%) as a yellow solid.

¹H NMR (400 MHz, DMSO-d₆) ppm=8.66 (d, J=2.8 Hz, 1H), 8.55 (t, J=5.4 Hz, 1H), 8.34 (br s, 2H), 8.21 (dd, J=9.5, 2.7 Hz, 1H), 6.96 (d, J=9.6 Hz, 1H), 4.13-4.05 (m, 1H), 3.88 (s, 3H), 3.76 (s, 3H), 3.37 (q, J=6.2 Hz, 2H), 1.89-1.75 (m, 2H), 1.69-1.58 (m, 2H), 1.56-1.32 (m, 2H).

MS (ESI⁺) calc. for C₁₅H₁₂N₃O₆⁺ [M+H]⁺ 340.15, found 340.44.

### Methyl (S)-2-((5-(4-(N-((2,4-diaminopteridin-6-yl)methyl)formamido)benzamido)-6-methoxy-6-oxohexyl)amino)-5-nitrobenzoate (XT59)

Amine XT58 (310 mg, 0.74 mmol) was reacted with acid XT7 (150 mg, 0.74 mmol) according to general procedure XXA. The product was recrystallized from MeOH (8 mL) to yield XT59 (390 mg, 80%) as an orange solid.

¹H NMR (400 MHz, DMSO-d₆) ppm=8.80 (s, 1H), 8.71-8.67 (m, 1H), 8.66 (s, 1H), 8.60 (d, J=2.8 Hz, 1H), 8.54 (t, J=5.3 Hz, 1H), 8.15 (dd, J=9.5, 2.8 Hz, 1H), 7.87 (d, J=8.5 Hz, 2H), 7.65 (br s, 1H), 7.58 (d, J=8.5 Hz, 2H), 7.33 (br s, 1H), 6.92 (d, J=9.5 Hz, 1H), 6.63 (br s, 2H), 5.22 (s, 2H), 4.46-4.38 (m, 1H), 3.82 (s, 3H), 3.62 (s, 3H), 3.40-3.27 (m, 2H), 1.90-1.78 (m, 2H), 1.71-1.55 (m, 2H), 1.54-1.37 (m, 2H).

MS (ESI⁺) calc. for C₃₀H₃₃N₁₀O₈⁺ [M+H]⁺ 661.25, found 661.65.

### Methyl (S)-5-amino-2-((5-(4-(N-((2,4-diaminopteridin-6-yl)methyl)formamido) benzamido)-6-methoxy-6-oxohexyl)amino)benzoate (XT60)

To a solution of XT59 (150 mg, 0.23 mmol) in DMF (1.5 mL) was added sat. aq. NH₄Cl (0.375 mL) and zinc powder (445 mg, 6.81 mmol). The resulting suspension was stirred for 2 h at RT. Next, the reaction mixture was diluted with DMF (4 mL) and filtered over Celite. The filtrate was stirred under air at RT for 18 h. After stirring for 16 h, the reaction mixture was concentrated and the crude product was suspended in MeOH (6 mL), filtered and the residue washed with ether (4 mL) to give aniline XT60 (145 mg, quant) as a grey solid.

¹H NMR (400 MHz, DMSO-d₆) ppm=8.80 (s, 1H), 8.70 (d, J=7.6 Hz, 1H), 8.66 (s, 1H), 7.88 (d, J=8.5 Hz, 2H), 7.73 (br s, 1H), 7.58 (d, J=8.5 Hz, 2H), 7.54 (t, J=8.7 Hz, 1H), 7.10 (d, J=2.6 Hz, 1H), 6.92 (t, J=4.8 Hz, 1H), 6.80 (dd, J=8.7, 2.5 Hz, 1H), 6.72 (bs s, 2H), 6.56 (d, J=8.8 Hz, 1H), 5.22 (s, 2H), 4.58 (s, 2H), 4.40 (q, J=7.1 Hz, 1H), 3.70 (s, 3H), 3.63 (s, 3H), 3.07 (q, J=5.7 Hz, 2H), 1.88-1.72 (m, 2H), 1.66-1.35 (m, 4H).

MS (ESI⁺) calc. for C₃₀H₃₅N₁₀O₆⁺ [M+H]⁺ 631.27, found 631.15.

### Methyl 5-((S)-2-((S)-2-((tert-butoxycarbonyl)amino)-3-methylbutanamido) propanamido)-2-(((S)-5-(4-(N-((2,4-diaminopteridin-6-yl)methyl)formamido)benzamido)-6-methoxy-6-oxohexyl)amino)benzoate (XT61)

Aniline XT60 (140 mg, 0.22 mmol) was reacted with Fmoc-Ala-OH (70 mg, 0.22 mmol) according to general procedure XXA. The product was purified by flash chromatography (silica gel, MeOH:DCM 0:1 to 1:3) to give the resulting amide (60 mg, 82%). A portion of the product (40 mg, 0.04 mmol) was deprotected with piperidine and reacted with Boc-Val-OSu according to the procedure for XT43. The product was purified by flash chromatography (silica gel, MeOH:DCM 0:1 to 1:3) to give XT61 (30 mg, 77%) as a yellow solid.

MS (ESI⁺) calc. for C₄₃H₅₇N₁₂O₁₀⁺ [M+H]⁺ 901.43, found 901.54.

### 5-((S)-2-((S)-2-Amino-3-methylbutanamido)propanamido)-2-(((S)-5-carboxy-5-(4-(((2,4-diaminopteridin-6-yl)methyl)amino)benzamido)pentyl)amino)benzoic acid (XT62)

Carbamate XT61 (15 mg, 0.02 mmol) was suspended in DCM (1.5 mL), cooled to 0° C. and TFA (1.5 mL) was added. The resulting mixture was stirred at 0° C. for 15 min and was then concentrated and coevaporated with DCM. The crude was subsequently hydrolyzed with NaOH (24 eq) according to general procedure XXC, with the following modification. Upon treatment with 1 M AcOH the product did not readily precipitate. Methanol was removed by evaporation in vacuo, and the aq. solution was lyophilized. The resulting cake was stirred with DMF (5 mL), filtered, and the obtained DMF solution containing XT62 was used directly in the next step.

MS (ESI⁺) calc. for C₃₅H₄₅N₁₂O₇⁺ [M+H]⁺ 745.35, found 745.52.

### 2-(((S)-5-Carboxy-5-(4-(((2,4-diaminopteridin-6-yl)methyl)amino)benzamido)pentyl) amino)-5-((S)-2-((S)-2-(6-(2,5-dioxo-2,5-dihydro-1H-pyrrol-1-yl)hexanamido)-3-methylbutanamido) propanamido)benzoic acid (XT63)

Amine XT62 (25 mg, 0.034 mmol) was reacted with 6-maleimidohexanoic acid N-hydroxylsuccinimide ester (10 mg, 0.034 mmol) and DIPEA (0.06 mL, 0.34 mmol) according to the procedure for XT17. Purification by preparative RP-HPLC (water×0.1% TFA/MeCN×0.1% TFA, gradient 20% to 30%) gave XT63 (8 mg, 25%) as a yellow solid.

¹H NMR (400 MHz, DMSO-d₆) ppm=13.25 (br s, 1H), 12.43 (br s, 1H), 9.67 (s, 1H), 9.38-9.13 (m, 3H), 8.84 (s, 1H), 8.17 (d, J=7.7 Hz, 1H), 8.08 (d, J=7.1 Hz, 1H), 8.03 (d, J=2.6 Hz, 1H), 7.83 (d, J=8.6 Hz, 1H), 7.73 (d, J=8.7 Hz, 2H), 7.57 (dd, J=9.1, 2.4 Hz, 1H), 6.99 (s, 2H), 6.95 (br s, 1H), 6.76 (d, J=8.7 Hz, 2H), 6.68 (d, J=9.3 Hz, 1H), 6.53 (br s, 2H), 4.61 (s, 2H), 4.38-4.28 (m, 2H), 4.18-4.12 (m, 1H), 3.17-3.07 (m, 4H), 2.22-2.06 (m, 2H), 2.01-1.90 (m, 1H), 1.88-1.75 (m, 2H), 1.68-1.36 (m, 8H), 1.34-1.23 (m, 3H), 1.23-1.12 (m, 2H), 0.86 (d, J=6.7 Hz, 3H), 0.82 (d, J=6.7 Hz, 3H).

MS (ESI⁺) calc. for C₄₅H₅₆N₁₃O₁₀⁺ [M+H]⁺ 938.43, found 938.70.

### Preparation of 5-(((S)-4-carboxy-4-(4-(((2,4-diaminopteridin-6-yl)methyl)amino) benzamido)butyl)-amino)-2-((S)-2-((S)-2-(6-(2,5-dioxo-2,5-dihydro-1H-pyrrol-1-yl)hexanamido)-3-methylbutanamido)propanamido)benzoic acid (XT723)

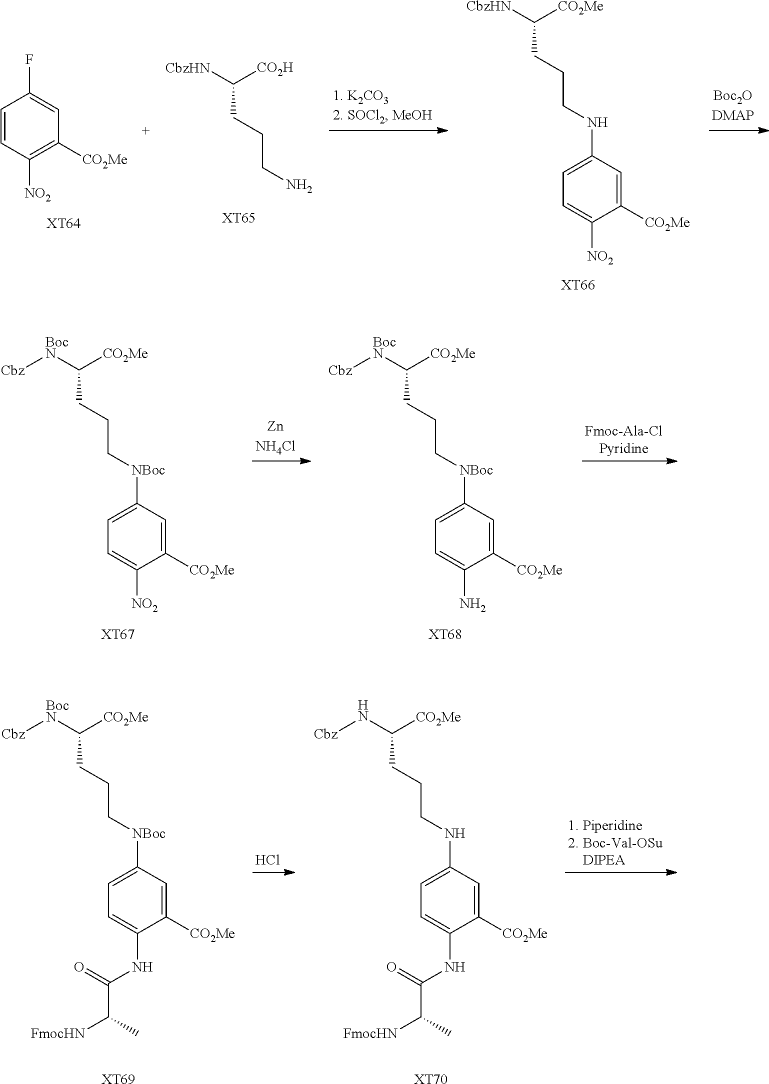

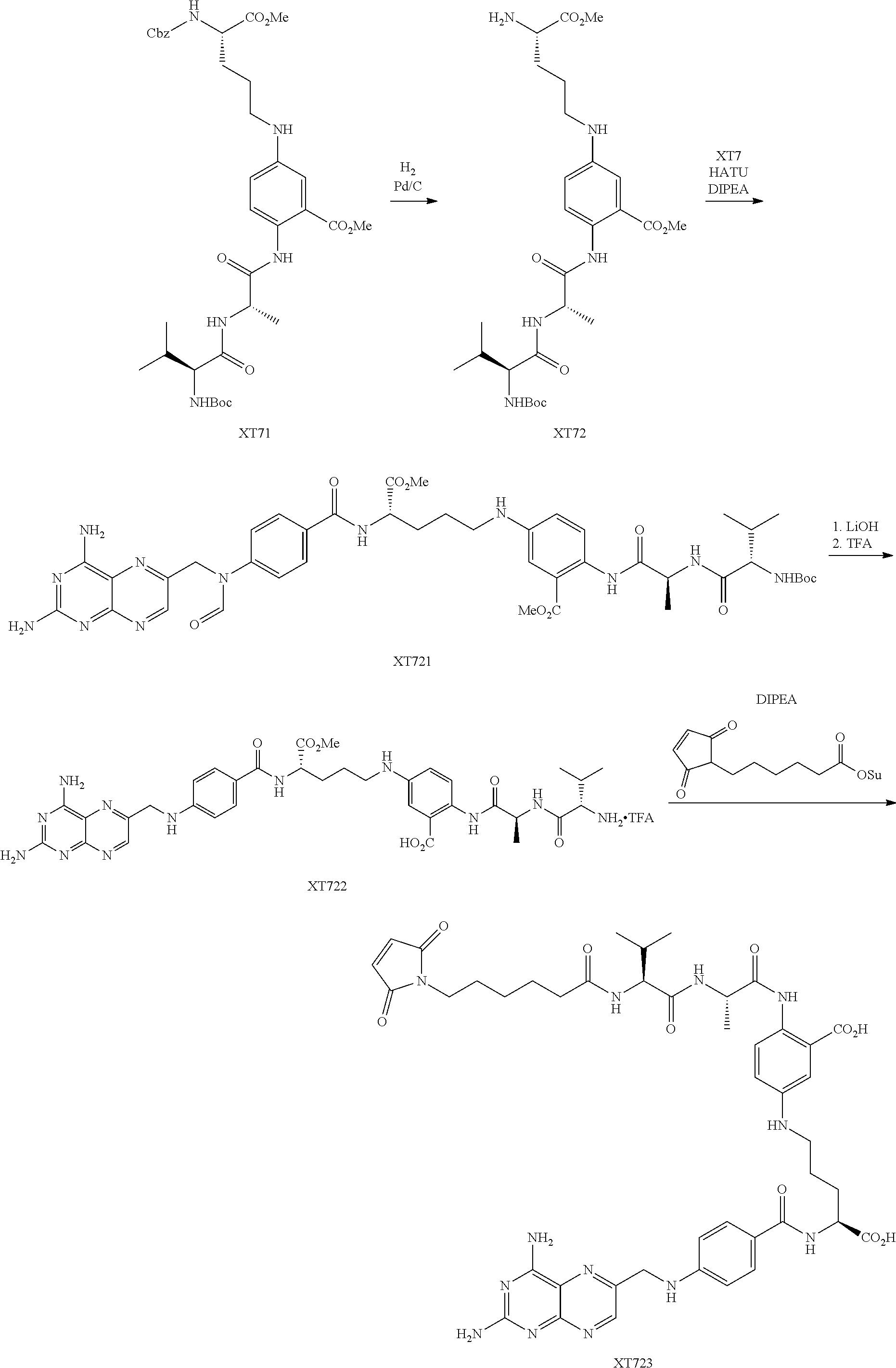

### Methyl (S)-5-((4-(((benzyloxy)carbonyl)amino)-5-methoxy-5-oxopentyl)amino)-2-nitrobenzoate (XT66)

XT64 (1.50 g, 7.51 mmol) was reacted with XT65 (2.00 g, 7.51 mmol) according to the procedure for XT57. The reaction time of the first step was extended to 7 days. Purification by flash chromatography (silica gel, MeOH:DCM 0:1 to 1:9) afforded XT66 (2.00 g, 58%) as a yellow/orange oil.

¹H NMR (400 MHz, DMSO-d₆) ppm=7.95 (d, J=9.2 Hz, 1H), 7.79 (d, J=7.7 Hz, 1H), 7.40-7.26 (m, 5H), 6.70 (dd, J=9.3, 2.2 Hz, 1H), 6.65 (d, J=2.2 Hz, 1H), 5.03 (s, 2H), 4.85 (br s, 1H), 4.12-4.05 (m, 1H), 3.81 (s, 3H), 3.63 (s, 3H), 3.18-3.11 (m, 2H), 1.86-1.74 (m, 1H), 1.73-1.54 (m, 3H).

MS (ESI⁺) calc. for C₂₂H₂₆N₃O₈⁺ [M+H]⁺ 460.17, found 460.33.

### Methyl (S)-5-((4-(((benzyloxy)carbonyl)(tert-butoxycarbonyl)amino)-5-methoxy-5-oxopentyl)(tert-butoxycarbonyl)amino)-2-nitrobenzoate (XT67)

To a solution of aniline XT66 (2.00 g, 4.35 mmol) in THF (25 mL) were added Boc₂O (3.03 mL, 13.06 mmol) and 4-dimethylaminopyridine (DMAP; 53 mg, 0.44 mmol). The resulting mixture was stirred at RT for 18 h. The reaction mixture was subsequently concentrated and the crude product was purified by flash chromatography (silica gel, EtOAc:DCM 0:1 to 1:3) to give the bis-Boc protected product XT67 (1.90 g, 66%) as a yellow oil.

MS (ESI⁺) calc. for C₂₂H₂₆N₃O₈⁺ [M−2×Boc+H]⁺460.17, found 460.30.

### Methyl (S)-2-amino-5-((4-(((benzyloxy)carbonyl)(tert-butoxycarbonyl)amino)-5-methoxy-5-oxopentyl)(tert-butoxycarbonyl)amino)benzoate (XT68)

Compound XT67 (1.70 g, 2.58 mmol) was reacted with zinc powder (2.50 g, 38.7 mmol) according to the procedure for XT60, with the modification that the crude product was purified by flash chromatography (silica gel, EtOAc:DCM 0:1 to 1:1) to give aniline XT68 (1.5 g, 92%) as an orange foam.

¹H NMR (400 MHz, DMSO-d₆) ppm=7.44 (d, J=2.3 Hz, 1H), 7.39-7.31 (m, 5H), 7.03 (dd, J=8.7, 2.1 Hz, 1H), 6.74 (d, J=8.9 Hz, 1H), 6.67 (br s, 2H), 5.18 (s, 2H), 4.93 (dd, J=10.0, 4.7 Hz, 1H), 3.76 (s, 3H), 3.50-3.50 (m, 1H), 3.58 (s, 3H), 3.44-3.34 (m, 1H), 2.04-1.94 (m, 1H), 1.86-1.73 (m, 1H), 1.47-1.28 (m, 2H), 1.34 (s, 18H).

MS (ESI⁺) calc. for C₃₂H₄₄N₃O₁₀⁺ [M+H]⁺ 630.30, found 630.40.

### Methyl 2-((S)-2-((((9H-fluoren-9-yl)methoxy)carbonyl)amino)propanamido)-5-(((S)-4-(((benzyloxy)carbonyl)(tert-butoxycarbonyl)amino)-5-methoxy-5-oxopentyl)(tert-butoxy-carbonyl)amino)benzoate (XT69)

Aniline XT68 (1.43 g, 2.27 mmol) was reacted with Fmoc-Ala-Cl (824 mg, 2.50 mmol) according to the procedure for XT11. Purification by flash chromatography (silica gel, EtOAc:DCM 0:1 to 1:3) afforded amide XT69 (2.1 g, quant) as a yellow foam.

¹H NMR (400 MHz, DMSO-d₆) ppm=11.19 (s, 1H), 8.61-8.56 (m, 1H), 8.48 (d, J=9.0 Hz, 1H), 8.08 (d, J=6.3 Hz, 1H), 7.89 (d, J=7.4 Hz, 2H), 7.83-7.69 (m, 3H), 7.48-7.27 (m, 9H), 5.17 (s, 2H), 4.92 (dd, J=9.8, 4.8 Hz, 1H), 4.47-4.40 (m, 1H), 4.36-4.22 (m, 2H), 4.19-4.09 (m, 1H), 3.72 (s, 3H), 3.30-3.63 (m, 1H), 3.59-3.50 (m, 1H), 3.58 (s, 3H), 2.06-1.96 (m, 1H), 1.86-1.74 (m, 1H), 1.43-1.29 (m, 5H), 1.36 (s, 9H), 1.32 (s, 9H).

MS (ESI⁺) calc. for C₅₀H₅₉N₄O₁₃⁺ [M+H]⁺ 923.41, found 923.39.

### Methyl 2-((S)-2-((((9H-fluoren-9-yl)methoxy)carbonyl)amino)propanamido)-5-(((S)-4-(((benzyloxy)carbonyl)amino)-5-methoxy-5-oxopentyl)amino)benzoate (XT70)

To a solution of bis-Boc protected XT69 (2.10 g, 2.28 mmol) in dioxane (2 mL) was added HCl in dioxane (4 M, 12 mL) and the resulting mixture was stirred at RT for 1 h. The reaction mixture was concentrated and the crude product was purified by flash chromatography (silica gel, MeOH:DCM 0:1 to 1:9) to afford aniline XT70 (1.45 g, 88%) as a yellow foam.

¹H NMR (400 MHz, DMSO-d₆) ppm=10.69 (s, 1H), 8.13 (d, J=9.0 Hz, 1H), 7.95 (d, J=6.7 Hz, 1H), 7.90 (d, J=7.5 Hz, 2H), 7.82-7.72 (m, 3H), 7.45-7.25 (m, 10H), 7.10 (d, J=2.6 Hz, 1H), 6.83 (dd, J=8.9, 2.7 Hz, 1H), 5.04 (s, 2H), 4.44-4.35 (m, 1H), 4.31-4.23 (m, 2H), 4.14-4.02 (m, 2H), 3.69 (s, 3H), 3.63 (s, 3H), 3.03-2.93 (m, 2H) 1.86-1.75 (m, 1H), 1.72-1.54 (m, 3H), 1.34 (d, J=7.2 Hz, 3H).

MS (ESI⁺) calc. for C₄₀H₄₃N₄O₉⁺ [M+H]⁺ 723.30, found 723.51.

### Methyl 5-(((S)-4-(((benzyloxy)carbonyl)amino)-5-methoxy-5-oxopentyl)amino)-2-((S)-2-((S)-2-((tert-butoxycarbonyl)amino)-3-methylbutanamido)propanamido)benzoate (XT71)

Fmoc-protected amine XT70 (1.45 g, 2.00 mmol) was deprotected with piperidine and subsequently reacted with Boc-Val-OSu according to the procedure for XT43. Purification by flash chromatography (silica gel, MeOH:DCM 0:1 to 1:4) afforded dipeptide XT71 (600 mg, 43%) as a yellow solid.

¹H NMR (400 MHz, DMSO-d₆) ppm=10.23 (s, 1H), 8.20 (d, J=6.8 Hz, 1H), 7.85 (d, J=8.9 Hz, 1H), 7.77 (d, J=7.7 Hz, 1H), 7.42-7.26 (m, 5H), 7.04 (d, J=2.6 Hz, 1H), 6.78 (dd, J=8.9, 2.5 Hz, 1H), 6.67 (d, J=9.1 Hz, 1H), 5.77 (t, J=5.2 Hz, 1H), 5.04 (s, 2H), 4.43 (p, J=6.9 Hz, 1H), 4.10-4.01 (m, 1H), 3.94-3.88 (m, 1H), 3.80 (s, 3H), 3.63 (s, 3H), 2.97 (q, J=5.7 Hz, 2H), 2.09-1.97 (m, 1H), 1.86-1.75 (m, 1H), 1.72-1.52 (m, 3H), 1.38 (s, 9H), 1.31 (d, J=7.0 Hz, 3H), 0.85 (d, J=6.7 Hz, 3H), 0.77 (d, J=6.7 Hz, 3H).

¹³C NMR (100 MHz, DMSO-d₆) ppm=173.3, 171.9, 170.8, 168.0, 156.6, 156.0, 145.6, 137.4, 128.8, 128.3, 128.2, 123.8, 120.3, 117.3, 112.9, 78.5, 66.0, 59.7, 54.2, 52.6, 52.3, 50.0, 30.9, 28.8, 25.5, 19.8, 18.2, 18.1.

MS (ESI⁺) calc. for C₃₅H₅₀N₅₀O₁₀⁺ [M+H]⁺ 700.36, found 700.56.

### Methyl 5-(((S)-4-amino-5-methoxy-5-oxopentyl)amino)-2-((S)-2-((S)-2-((tert-butoxycarbonyl) amino)-3-methylbutanamido)propanamido)benzoate (XT72)

Compound XT71 (600 mg, 0.86 mmol) was dissolved in DMF (8.5 mL) under N₂ atmosphere. Pd/C (91 mg, 10 mol % on activated carbon) was added and the resulting black suspension was vigorously stirred at RT under hydrogen atmosphere for 90 min. The flask was purged with N₂, and the reaction mixture was filtered over Celite. Concentration of the filtrate to afforded XT71 (quant) as a dark green solid.

MS (ESI⁺) calc. for C₂₇H₄₄N₅₀⁺ [M+H]⁺ 566.32, found 566.43.

### Methyl 2-((S)-2-((S)-2-((tert-butoxycarbonyl)amino)-3-methylbutanamido) propanamido)-5-(((S)-4-(4-(N-((2,4-diaminopteridin-6-yl)methyl)formamido)benzamido)-5-methoxy-5-oxopentyl)amino)benzoate (XT721)

Amine XT72 (167 mg, 0.295 mmol) was reacted with acid XT7 (100 mg, 0.295 mmol) according to general procedure XXA. The product was recrystallized from MeOH (5 mL) to yield XT721 (216 mg, 83%) as an orange solid.

¹H NMR (400 MHz, DMSO-d₆) ppm=10.23 (s, 1H), 8.80 (s, 1H), 8.72 (d, J=7.5 Hz, 1H), 8.67 (s, 1H), 8.20 (d, J=6.8 Hz, 1H), 7.87 (d, J=8.7 Hz, 2H), 7.83 (d, J=9.0 Hz, 2H), 7.79 (d, J=8.7 Hz, 2H), 7.47 (br s, 1H), 7.04 (d, J=2.8 Hz, 1H), 6.78 (dd, J=9.0, 2.7 Hz, 1H), 6.74 (br s, 2H), 6.67 (d, J=9.2 Hz, 1H), 5.78 (br s, 1H), 5.23 (s, 2H), 4.47-4.39 (m, 1H), 4.33 (p, J=7.0 Hz, 1H), 3.94-3.89 (m, 1H), 3.79 (s, 3H), 3.63 (s, 3H), 3.04-2.95 (m, 2H), 2.09-1.98 (m, 1H), 1.98-1.88 (m, 1H), 1.88-1.77 (m, 1H), 1.69-1.55 (m, 2H), 1.38 (s, 9H), 1.31 (d, J=7.1 Hz, 3H), 0.85 (d, J=6.7 Hz, 3H), 0.77 (d, J=6.7 Hz, 3H).

MS (ESI⁺) calc. for C₄₂H₅₅N₁₂O₁₀⁺ [M+H]⁺ 887.42, found 887.90.

### 2-((S)-2-((S)-2-Amino-3-methylbutanamido)propanamido)-5-(((S)-4-carboxy-4-(4-(((2,4-diaminopteridin-6-yl)methyl)amino)benzamido)butyl)amino)benzoic acid (XT722)

To a solution of ester XT721 (210 mg, 0.24 mmol) in THF (1.5 mL) was added a solution of LiOH (28 mg, 1.2 mmol) in water (1.5 mL). The resulting mixture was stirred for 72 h, with overnight storage at −78° C. Upon completion, the reaction mixture was acidified with aq. AcOH (1.0 M, 2.5 mL) and the resulting suspension was stirred at RT for 60 min. The mixture was filtered and the residue was washed with water (10 mL), MeOH (4 mL) and ether (10 mL). The obtained solid was dried on air overnight to give a yellow solid (150 mg). Next, the material was suspended in DCM (3 mL), cooled to 0° C. and TFA (3 mL) was added. The resulting solution was stirred for 15 min. whilst warming to RT. The reaction mixture was concentrated and coevaporated with DCM, to afford TFA-salt XT722 (quant) which was directly used in the next reaction.

MS (ESI⁺) calc. for C₃₄H₄₃N₁₂O₇⁺ [M+H]⁺ 731.34, found 731.52.

### 5-(((S)-4-Carboxy-4-(4-(((2,4-diaminopteridin-6-yl)methyl)amino)benzamido)butyl) amino)-2-((S)-2-((S)-2-(6-(2,5-dioxo-2,5-dihydro-1H-pyrrol-1-yl)hexanamido)-3-methyl-butanamido) propanamido)benzoic acid (XT723)

Amine XT722 (132 mg, 0.18 mmol) was reacted with 6-maleimidohexanoic acid N-hydroxylsuccinimide ester (56 mg, 0.18 mmol) and DIPEA (0.314 mL, 1.80 mmol) according to the procedure for XT17. Purification of the residue by trituration in MeOH, followed by filtration and preparative RP-HPLC (water×0.1% TFA/MeCN×0.1% TFA, gradient 20% to 50%) gave XT723 (72 mg, 43%, 3 steps) as a yellow solid.

¹H NMR (400 MHz, DMSO-d₆) ppm=13.67-12.00 (m, 2H), 10.77 (s, 1H), 9.35 (br s, 1H), 9.31 (br s, 1H), 8.84 (s, 1H), 8.64 (br s 1H), 8.34 (d, J=6.5 Hz, 1H), 8.21-8.13 (m, 2H), 7.79-7.69 (m, 3H), 7.62 (br s, 1H), 7.19 (d, J=2.4 Hz, 1H), 7.00 (s, 2H), 6.83 (dd, J=9.0, 2.3 Hz, 1H), 6.75 (d, J=8.8 Hz, 2H), 4.63 (s, 2H), 4.39-4.32 (m, 1H), 4.31-4.20 (m, 2H), 3.36 (t, J=7.0 Hz, 2H), 3.01 (t, J=6.8 Hz, 2H), 2.23-2.02 (m, 3H), 1.98-1.87 (m, 1H), 1.87-1.74 (m, 1H), 1.70-1.56 (m, 2H), 1.53-1.41 (m, 4H), 1.31 (d, J=7.1 Hz, 3H), 1.23-1.13 (m, 2H), 0.83 (d, J=6.7 Hz, 3H), 0.76 (d, J=6.7 Hz, 3H).

MS (ESI⁺) calc. for C₄₄H₅₄N₁₃O₁₀⁺ [M+H]⁺ 924.41, found 924.45.

### Preparation of (S)-2-(4-(((2,4-diaminopteridin-6-yl)methyl)amino)benzamido)-5-((4-((S)-2-((S)-2-(6-(2,5-dioxo-2,5-dihydro-1H-pyrrol-1-yl)hexanamido)-3-methylbutanamido) propanamido)phenyl)amino)pentanoic acid (XT80)

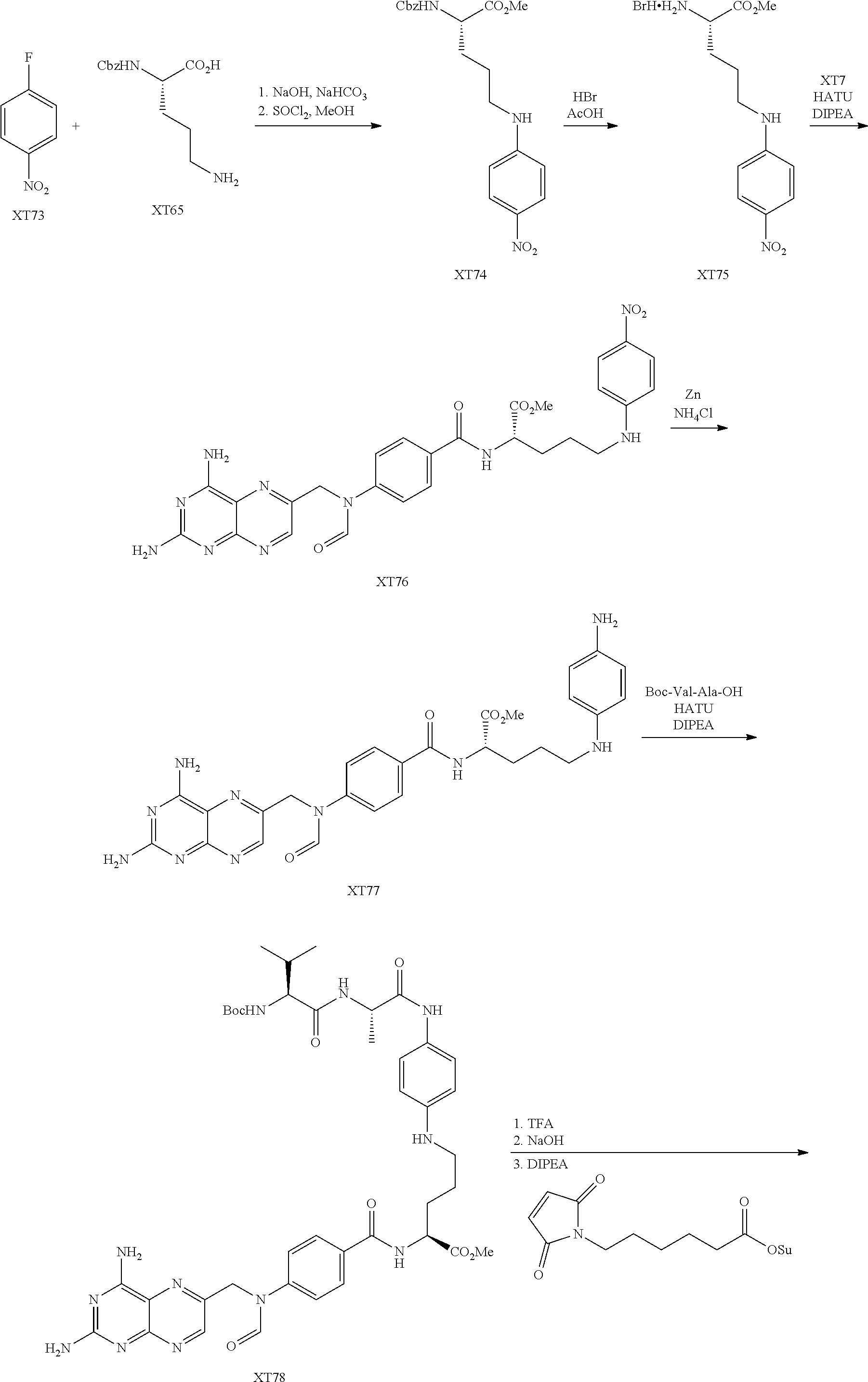

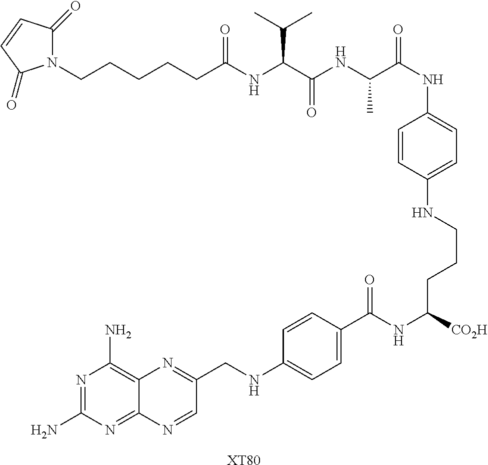

### Methyl (S)-2-(((benzyloxy)carbonyl)amino)-5-((4-nitrophenyl)amino)pentanoate (XT74)

A mixture of XT73 (795 mg, 5.63 mmol), XT65 (500 mg, 1.88 mmol), NaOH (75 mg, 1.88 mmol) and NaHCO₃ (473 mg, 5.63 mmol) in water (10 mL)/EtOH (6 mL) in a sealed tube was heated to 90° C. for 5 days. After cooling to RT, the reaction mixture was diluted with water (20 mL) and washed with ether (40 mL). The aq. solution was then acidified with 6 M HCl to pH ˜3 and the resulting mixture was extracted with 5% MeOH in DCM (2×75 mL). The combined organic extracts were washed with water, dried over Na₂SO₄, filtered and concentrated to yield the intermediate carboxylic acid (610 mg, 84%) as a yellow foam. The material was suspended in MeOH (50 mL), cooled to 0° C. and thionyl chloride was (1.26 mL, 17.3 mmol) added dropwise. The resulting mixture was stirred at 0° C. for 45 min and was then allowed to reach RT over 1 h. Upon completion, the reaction mixture was concentrated and the crude product was purified by flash chromatography (silica gel, MeOH:DCM 0:1 to 1:9) to give ester XT74 (670 mg, quant) as a yellow foam.

¹H NMR (400 MHz, DMSO-d₆) ppm=7.98 (d, J=9.2 Hz, 2H), 7.78 (d, J=7.8 Hz, 1H), 7.39-7.25 (m, 6H), 6.62 (d, J=9.3 Hz, 2H), 5.04 (s, 2H), 4.12-.4.04 (m, 1H), 3.10 (s, 3H), 3.17-3.08 (m, 2H), 1.87-1.74 (m, 1H), 1.72-1.56 (m, 3H).

MS (ESI⁺) calc. for C₂₀H₂₄N₃O₆⁺ [M+H]⁺ 402.17, found 402.33.

### Methyl (S)-2-amino-5-((4-nitrophenyl)amino)pentanoate hydrobromide (XT75)

Ester XT74 (480 mg, 1.20 mmol) was deprotected according to the procedure for XT58. The reaction mixture was subsequently concentrated and coevaporated with toluene. The residue was taken up in water and lyophilized to give a sticky gum. The material was stirred in ether until to give a suspension, that was then filtered. The residue was coevaporated with MeOH to give amine XT75 (550 mg quant) as a yellow foam.

¹H NMR (400 MHz, DMSO-d₆) ppm=8.41 (br s, 3H), 8.00 (d, J=9.2 Hz, 2H), 6.66 (d, J=9.2 Hz, 2H), 4.13 (sextet, J=5.5 Hz, 1H), 3.75 (s, 3H), 3.19 (t, J=6.8 Hz, 2H), 1.96-1.79 (m, 2H), 1.77-1.53 (m, 2H).

MS (ESI⁺) calc. for C₁₂H₁₈N₃O₄⁺ [M+H]⁺ 268.13, found 268.27.

### Methyl (S)-2-(4-(N-((2,4-diaminopteridin-6-yl)methyl)formamido)benzamido)-5-((4-nitrophenyl)-amino)pentanoate (XT76)

Amine XT75 (250 mg, 0.718 mmol) was reacted with acid XT7 (244 mg, 0.718 mmol) according to general procedure XXA. The product was recrystallized from MeOH (8 mL) to yield XT76 (310 mg, 73%) as an orange solid.

¹H NMR (400 MHz, DMSO-d₆) ppm=8.80 (s, 1H), 8.74 (d, J=7.5 Hz, 1H), 8.65 (s, 1H), 7.96 (d, J=8.9 Hz, 2H), 7.88 (d, J=8.7 Hz, 2H), 7.66 (br s, 1H), 7.59 (d, J=8.7 Hz, 2H), 7.34 (br s, 1H), 7.29 (t, J=5.1 Hz, 1H), 6.62 (br s, 2H), 6.61 (d, J=9.2 Hz, 2H), 5.21 (s, 2H), 4.51-4.41 (m, 1H), 3.63 (s, 3H) 3.20-3.12 (m, 2H), 1.99-1.75 (m, 2H), 1.73-1.57 (m, 2H).

MS (ESI⁺) calc. for C₂₇H₂₉N₁₀O₆⁺ [M+H]⁺ 589.23, found 589.59.

### Methyl (S)-5-((4-aminophenyl)amino)-2-(4-(N-((2,4-diaminopteridin-6-yl)methyl) formamido)-benzamido)pentanoate (XT77)

Nitroaniline XT76 (340 mg, 0.578 mmol) was reduced using zinc powder (1133 mg, 17.3 mmol) according to the procedure for XT60, to yield aniline XT77 (305 mg, 95%) as a grey solid.

MS (ESI⁺) calc. for C₂₇H₃₁N₁₀O₄⁺ [M+H]⁺ 559.25, found 559.14.

### Methyl (S)-5-((4-((S)-2-((S)-2-((tert-butoxycarbonyl)amino)-3-methylbutanamido) propanamido)-phenyl)amino)-2-(4-(N-((2,4-diaminopteridin-6-yl)methyl)formamido) benzamido)pentanoate (XT78)

Aniline XT77 (300 mg, 0.54 mmol) was reacted with Boc-ValAla-OH (170 mg, 0.59 mmol) according to general procedure XXA. Purification by flash chromatography (silica gel, MeOH:DCM 0:1 to 1:3) afforded XT78 (240 mg, 54%) as a yellow solid.

¹H NMR (400 MHz, DMSO-d₆) ppm=9.55 (s, 1H), 8.81 (s, 1H), 8.78-8.68 (m, 3H), 8.52 (d, J=8.4 Hz, 1H), 8.43 (br s, 1H), 8.12 (br s, 2H), 7.95 (d, J=7.1 Hz, 1H), 7.90 (d, J=8.6 Hz, 2H), 7.60 (d, J=8.6 Hz, 2H), 7.25 (d, J=8.8 Hz, 2H), 6.75 (d, J=8.9 Hz, 1H), 6.48 (d, J=8.8 Hz, 2H), 5.27 (s, 2H), 4.48-4.34 (m, 2H), 3.89-3.78 (m, 1H), 3.63 (s, 3H) 2.97 (t, J=6.6 Hz, 2H), 2.03-1.74 (m, 3H), 1.69-1.51 (m, 2H), 1.38 (s, 9H), 1.31-1.19 (m, 3H), 0.90-0.78 (m, 6H).

MS (ESI⁺) calc. for C₄₀H₅₃N₁₂O₈⁺ [M+H]⁺ 829.41, found 829.58.

### (S)-2-(4-(((2,4-Diaminopteridin-6-yl)methyl)amino)benzamido)-5-((4-((S)-2-((S)-2-(6-(2,5-dioxo-2,5-dihydro-1H-pyrrol-1-yl)hexanamido)-3-methylbutanamido)propanamido) phenyl)amino)pentanoic acid (XT80)

Carbamate XT78 (240 mg, 0.29 mmol) was suspended in DCM (3 mL), cooled to 0° C. and TFA (3 mL) was added. The resulting mixture was stirred at 0° C. for 15 min and was then concentrated and coevaporated with DCM. The hydrolysis of the intermediate ester with NaOH (36 eq) was carried out according to general procedure XXC, with the modification that upon treatment with 1 M AcOH the product did not solidify sufficiently and therefore the resulting solution was lyophilized after evaporation of MeOH. The resulting cake was stirred with DMF (4 mL) and filtered. The filtrate, containing the deprotected product, was then reacted with 6-maleimidohexanoic acid N-hydroxylsuccinimide ester (31 mg, 0.100 mmol) and DIPEA (0.105 mL, 0.600 mmol) according to the procedure for XT17. The crude was triturated with MeOH, and after filtration the solid was purified by preparative RP-HPLC (water×0.1% TFA/MeCN×0.1% TFA, gradient 20% to 50%), to give XT80 (37 mg, 15%, 3 steps) as a yellow solid.

¹H NMR (400 MHz, DMSO-d₆) ppm=13.53-11.98 (m, 2H), 9.63 (br s, 1H), 9.36 (br s, 1H), 9.31 (br s, 1H), 8.84 (s, 1H), 8.62 (br s, 1H), 8.19 (d, J=7.8 Hz, 1H), 8.06 (d, J=7.0 Hz, 1H), 7.80 (d, J=8.6 Hz, 1H), 7.73 (d, J=8.6 Hz, 2H), 7.69 (br s, 1H), 7.38 (d, J=7.4 Hz, 2H), 6.99 (s, 2H), 6.88 (br s, 1H), 6.75 (d, J=8.6 Hz, 2H), 6.70 (br s, 2H), 4.62 (s, 2H), 4.41-4.30 (m, 2H), 4.18-4.11 (m, 1H), 3.41-3.31 (m, 2H), 3.11-2.98 (m, 2H), 2.22-2.06 (m, 2H), 2.01-1.86 (m, 2H), 1.86-1.74 (m, 1H), 1.71-1.56 (m, 2H), 1.54-1.40 (m, 4H), 1.28 (d, J=7.0 Hz, 3H), 1.22-1.13 (m, 2H), 0.85 (d, J=6.7 Hz, 3H), 0.82 (d, J=6.7 Hz, 3H).

MS (ESI⁺) calc. for C₄₃H₅₄N₁₃O₈⁺ [M+H]⁺ 880.42, found 880.52.

### Preparation of 2-(((S)-5-carboxy-5-(4-(((2,4-diaminopteridin-6-yl)methyl)amino) benzamido)-pentyl)amino)-5-((S)-2-((S)-2-(6-(2,5-dioxo-2,5-dihydro-1H-pyrrol-1-yl) hexanamido)-3-methylbutanamido)propanamido)nicotinic acid (XT89)

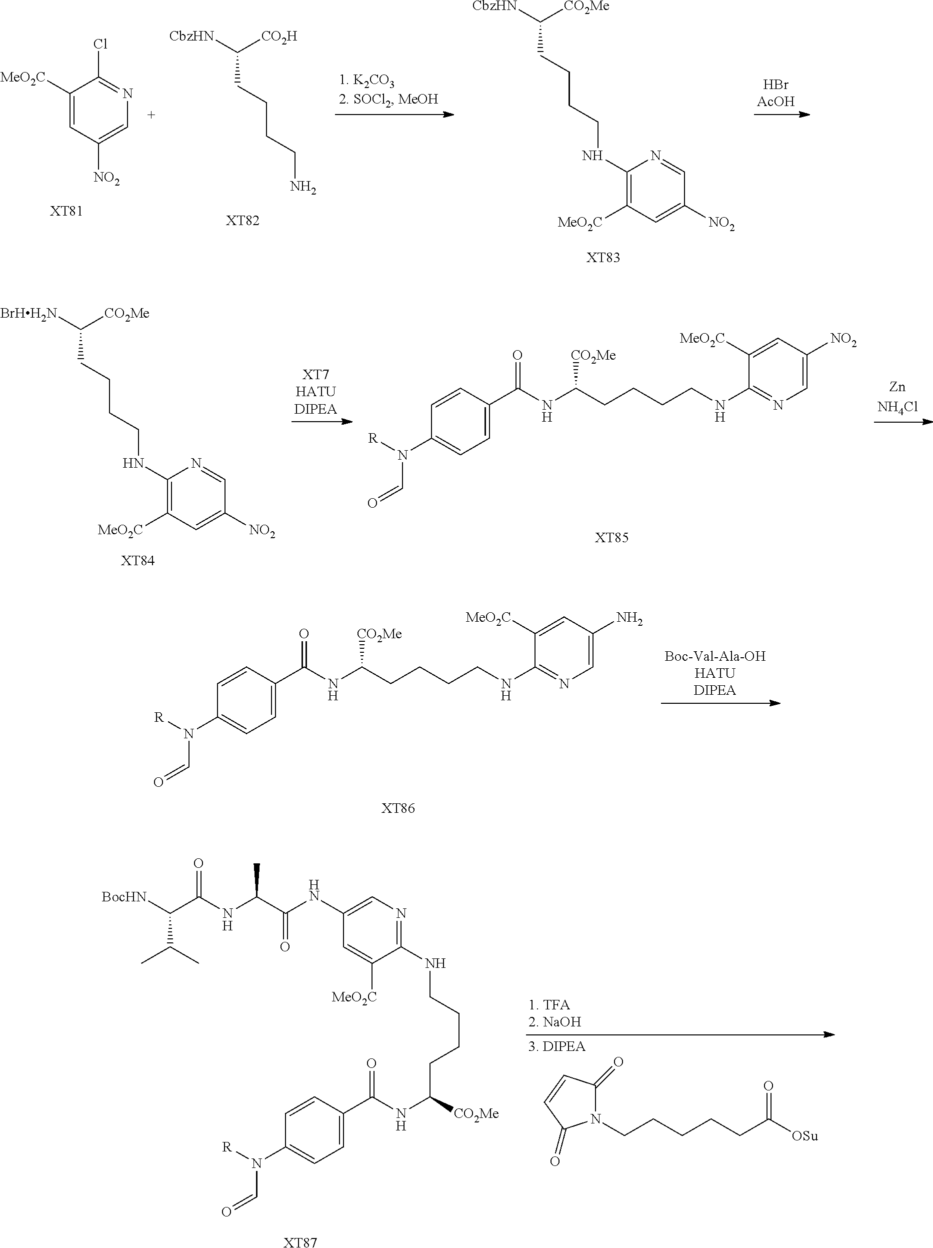

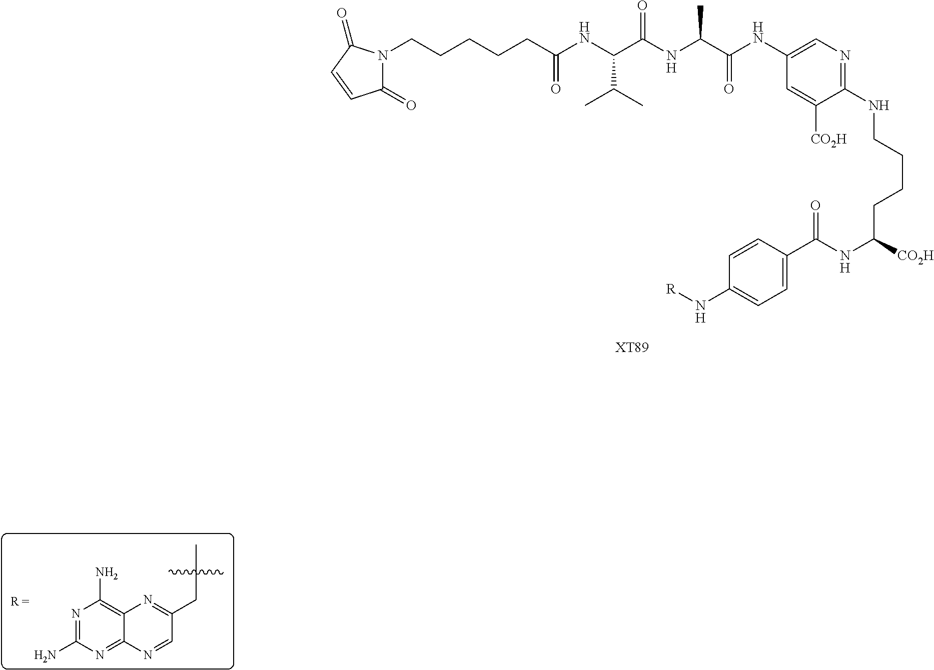

### Methyl (S)-2-((5-(((benzyloxy)carbonyl)amino)-6-methoxy-6-oxohexyl)amino)-5-nitronicotinate (XT83)

Amine XT82 (500 mg, 1.78 mmol) was reacted with XT81 (386 mg, 1.78 mmol) according to the procedure for XT57. The product was recrystallized from MeOH (8 mL) to yield XT83 (370 mg, 44%) as a yellow solid.

MS (ESI⁺) calc. for C₂₂H₂₇N₄O₈⁺ [M+H]⁺ 475.18, found 475.34.

### Methyl (S)-2-((5-amino-6-methoxy-6-oxohexyl)amino)-5-nitronicotinate hydrobromide (XT84)

Cbz-protected amine XT83 (370 mg, 0.78 mmol) was deprotected according to the procedure for XT58. The product was taken up in water and was then lyophilized. Suspending the residue in ether and filtration then afforded XT84 (quant) as a yellow solid.

¹H NMR (400 MHz, DMSO-d₆) ppm=9.12 (d, J=2.7 Hz, 1H), 8.85 (t, J=5.6 Hz, 1H), 8.70 (d, J=2.7 Hz, 1H), 8.40 (br s, 2H), 4.12-4.02 (m, 1H), 3.90 (s, 3H), 3.75 (s, 3H), 3.60 (q, J=6.5 Hz, 2H), 1.88-1.75 (m, 2H), 1.62 (p, J=7.1 Hz, 2H), 1.52-1.28 (m, 2H).

MS (ESI⁺) calc. for C₁₄H₂₁N₄O₆⁺ [M+H]⁺ 341.15, found 341.22.

### Methyl (S)-2-((5-(4-(N-((2,4-diaminopteridin-6-yl)methyl)formamido)benzamido)-6-methoxy-6-oxohexyl)amino)-5-nitronicotinate (XT85)

Amine XT84 (360 mg, 0.86 mmol) was reacted with acid XT7 (264 mg, 0.78 mmol) according to general procedure XXA. The product was recrystallized from MeOH (5 mL) to yield XT85 (280 mg, 55%) as an orange solid.

¹H NMR (400 MHz, DMSO-d₆) ppm=9.07 (d, J=2.7 Hz, 1H), 8.84 (t, J=5.7 Hz, 1H), 8.80 (s, 1H), 8.69-8.64 (m, 1H), 8.66 (s, 1H), 8.63 (d, J=2.7 Hz, 1H), 7.85 (d, J=8.6 Hz, 2H), 7.67 (br s, 1H), 7.57 (d, J=8.6 Hz, 2H), 7.35 (br s, 1H), 6.64 (br s, 2H), 5.22 (s, 2H), 4.45-4.35 (m, 1H), 3.84 (s, 3H), 3.66-3.50 (m, 2H), 3.61 (s, 3H), 1.89-1.75 (m, 2H), 1.69-1.51 (m, 2H), 1.50-1.32 (m, 2H).

MS (ESI⁺) calc. for C₂₉H₃₂N₁₁O₈⁺ [M+H]⁺ 662.24, found 662.44.

### Methyl (S)-5-amino-2-((5-(4-(N-((2,4-diaminopteridin-6-yl)methyl)formamido) benzamido)-6-methoxy-6-oxohexyl)amino)nicotinate (XT86)

Compound XT85 (280 mg, 0.42 mmol) was reduced using zinc powder (830 mg, 12.7 mmol) according to the procedure for XT60, to yield aniline XT86 (240 mg, 90%) as a grey solid.

¹H NMR (400 MHz, DMSO-d₆) ppm=8.80 (s, 1H), 8.68 (d, J=7.2 Hz, 1H), 8.66 (s, 1H), 7.87 (d, J=8.6 Hz, 2H), 7.81 (d, J=2.7 Hz, 1H), 7.68 (br s, 1H), 7.59 (d, J=8.6 Hz, 2H), 7.45 (d, J=2.8 Hz, 1H), 7.37 (br s, 1H), 7.22 (t, J=5.4 Hz, 1H), 6.66 (br s, 2H), 5.22 (s, 2H), 4.47 (s, 2H), 4.42-4.34 (m, 1H), 3.77-3.57 (m, 2H) 3.74 (s, 3H), 3.61 (s, 3H), 1.86-1.75 (m, 2H), 1.59-1.48 (m, 2H), 1.47-1.32 (m, 2H).

MS (ESI⁺) calc. for C₂₉H₃₄N₁₁O₆⁺ [M+H]⁺ 632.27, found 632.42.

### Methyl 5-((S)-2-((S)-2-((tert-butoxycarbonyl)amino)-3-methylbutanamido) propanamido)-2-(((S)-5-(4-(N-((2,4-diaminopteridin-6-yl)methyl)formamido)benzamido)-6-methoxy-6-oxohexyl)amino)nicotinate (XT87)

Aniline XT86 (120 mg, 0.19 mmol) was reacted with Boc-ValAla-OH (60 mg, 0.21 mmol) according to general procedure XXA. Purification by flash chromatography (silica gel, MeOH:DCM 0:1 to 1:3) afforded XT87 (100 mg, 58%) as a yellow solid.

MS (ESI⁺) calc. for C₄₂H₅₆N₁₃O₁₀⁺ [M+H]⁺ 902.43, found 902.49.

### 2-(((S)-5-Carboxy-5-(4-(((2,4-diaminopteridin-6-yl)methyl)amino)benzamido)pentyl) amino)-5-((S)-2-((S)-2-(6-(2,5-dioxo-2,5-dihydro-1H-pyrrol-1-yl)hexanamido)-3-methylbutanamido) propanamido)nicotinic acid (XT89)

Carbamate XT87 (100 mg, 0.11 mmol) was suspended in DCM (3 mL), cooled to 0° C. and TFA (3 mL) was added. The resulting mixture was stirred at 0° C. for 15 min, and was then concentrated and coevaporated with DCM. The hydrolysis of the intermediate ester with NaOH (18 eq) was carried out according to general procedure XXC. The residual cake was stirred with DMF (3 mL) and filtered. The filtrate was treated with 6-maleimidohexanoic acid N-hydroxylsuccinimide ester (31 mg, 0.10 mmol) and DIPEA (0.105 mL, 0.600 mmol) according to the procedure XT17. The mixture was concentrated and purified by preparative RP-HPLC (water×0.1% TFA/MeCN×0.1% TFA, gradient 20% to 30%), to give XT89 (12 mg, 13%, 3 steps) as a yellow solid.

¹H NMR (400 MHz, DMSO-d₆) ppm=13.60-12.03 (m, 2H), 9.79 (s, 1H), 9.35 (br s, 1H), 9.31 (br s, 1H), 8.85 (s, 1H), 8.62 (br s, 1H), 8.38 (d, J=2.7 Hz, 1H), 8.33 (d, J=2.7 Hz, 1H), 8.19-8.10 (m, 2H), 8.06-7.64 (m, 2H) 7.82 (d, J=8.5 Hz, 1H), 7.73 (d, J=8.7 Hz, 2H), 6.99 (s, 2H), 6.87 (br s, 1H), 6.74 (d, J=8.7 Hz, 2H), 4.62 (s, 2H), 4.37-4.28 (m, 2H), 4.18-4.11 (m, 1H), 3.43-3.30 (m, 4H), 2.23-2.06 (m, 2H), 2.01-1.90 (m, 1H), 1.87-1.73 (m, 2H), 1.63-1.52 (m, 2H), 1.52-1.35 (m, 6H), 1.29 (d, J=7.1 Hz, 3H), 1.22-1.13 (m, 2H), 0.86 (d, J=6.7 Hz, 3H), 0.82 (d, J=6.7 Hz, 3H).

MS (ESI⁺) calc. for C₄₄H₅₅N₁₄O₁₀⁺ [M+H]⁺ 939.42, found 939.37.

### Preparation of (S)-2-(4-(2-(2,4-diaminopteridin-6-yl)ethyl)benzamido)-5-(4-((S)-2-((S)-2-(6-(2,5-dioxo-2,5-dihydro-1H-pyrrol-1-yl)hexanamido)-3-methylbutanamido) propanamido)-2-(2H-tetrazol-5-yl)benzamido)pentanoic acid (XT94)

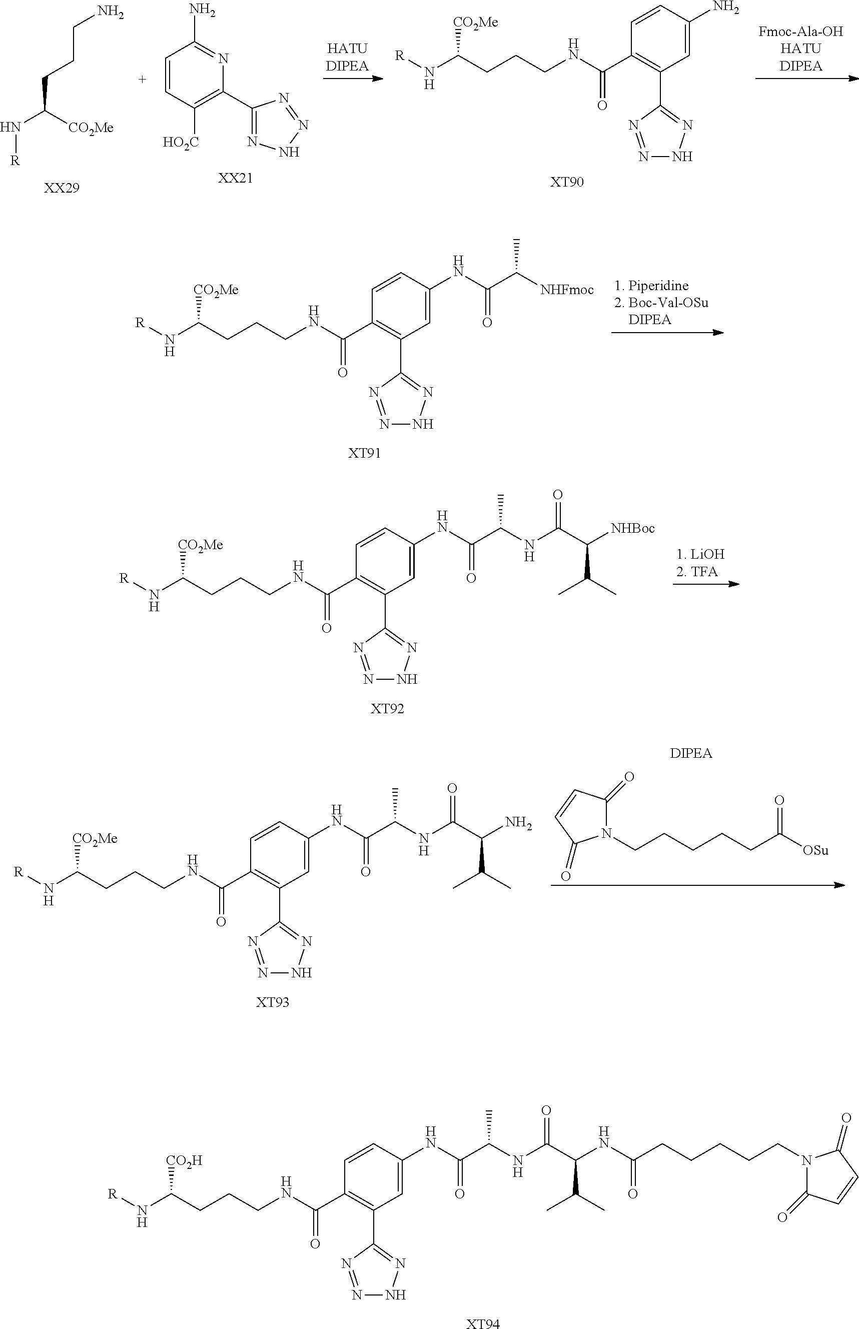

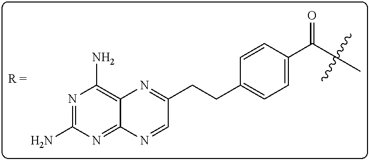 [SMILES: *C(=O)c1ccc(CCc2cnc3nc(N)nc(N)c3n2)cc1]

### Methyl (S)-5-(4-amino-2-(2H-tetrazol-5-yl)benzamido)-2-(4-(2-(2,4-diaminopteridin-6-yl) ethyl)benzamido)pentanoate (XT90)

Crude tetrazole XX21 (55 mg, 0.271 mmol) in DMF (1 mL) was reacted with amine XX29 (150 mg, 0.271 mmol) according to general procedure XXA. The product was recrystallized from MeOH (5 mL) and washed with ether (5 mL) to yield XT90 (quant) as an orange solid.

MS (ESI⁺) calc. for C₂₉H₃₂N₁₃O₄⁺ [M+H]⁺ 626.27, found 626.38.

### Methyl (S)-5-(4-((S)-2-((((9H-fluoren-9-yl)methoxy)carbonyl)amino)propanamido)-2-(2H-tetrazol-5-yl)benzamido)-2-(4-(2-(2,4-diaminopteridin-6-yl)ethyl)benzamido)pentanoate (XT91)

Aniline XT90 (150 mg, 0.240 mmol) was reacted with Fmoc-Ala-OH (112 mg, 0.360 mmol) according to general procedure XXA. The crude product was purified by flash chromatography (silica gel, MeOH:DCM×1% Et₃N 0:1 to 1:1). The isolated product was dissolved in MeOH and precipitated with ether. The resulting suspension was filtered, and the solid was washed with ether and dried on air overnight to yield amide XT91 (75 mg, 34%) as a yellow solid.

MS (ESI⁺) calc. for C₄₇H₄₇N₁₄O₇⁺ [M+H]⁺ 919.37, found 919.79.

### Methyl (S)-5-(4-((S)-2-((S)-2-((tert-butoxycarbonyl)amino)-3-methylbutanamido) propanamido)-2-(2H-tetrazol-5-yl)benzamido)-2-(4-(2-(2,4-diaminopteridin-6-yl)ethyl) benzamido)pentanoate (XT92)

Fmoc protected amine XT91 (75 mg, 0.08 mmol) was deprotected with piperidine (0.16 mL, 1.6 mmol) and subsequently reacted with Boc-Val-OSu (34 mg, 0.11 mmol) according to the procedure for XT43. Purification by precipitation from a minimal amount of methanol with ether, followed by filtration and drying of the residue on air afforded dipeptide XT92 (75 mg, 78%) as a yellow solid.

MS (ESI⁺) calc. for C₄₂H₅₄N₁₅O₈⁺ [M+H]⁺ 896.43, found 896.54.

### (S)-5-(4-((S)-2-((S)-2-Amino-3-methylbutanamido)propanamido)-2-(2H-tetrazol-5-yl) benzamido)-2-(4-(2-(2,4-diaminopteridin-6-yl)ethyl)benzamido)pentanoic acid (XT93)

To a solution of ester XT92 (75 mg, 0.084 mmol) in THF (1 mL) was added LiOH (10 mg, 0.419 mmol) in water (1 mL). The resulting solution was stirred at RT for 1 h, cooled to 0° C. and treated with AcOH (0.05 mL, 0.84 mmol). After stirring for 5 min, the suspension was concentrated and coevaporated with toluene. The residue was suspended in DCM (1 mL), cooled to 0° C. and TFA (1 mL) was added. The resulting solution was stirred for 60 min whilst gradually warming to RT. The reaction mixture was coevaporated with DCM to afford crude amine XT93 (quant).

MS (ESI⁺) calc. for C₃₆H₄₄N₁₅O₆⁺ [M+H]⁺ 782.36, found 782.73.

### (S)-2-(4-(2-(2,4-Diaminopteridin-6-yl)ethyl)benzamido)-5-(4-((S)-2-((S)-2-(6-(2,5-dioxo-2,5-dihydro-1H-pyrrol-1-yl)hexanamido)-3-methylbutanamido)propanamido)-2-(2H-tetrazol-5-yl)benzamido)pentanoic acid (XT94)

Crude amine XT93 (62.5 mg) was reacted with 6-maleimidohexanoic acid N-hydroxylsuccinimide ester (25 mg, 0.080 mmol) and DIPEA (0.140 mL, 0.800 mmol) according to procedure XT17. Purification by preparative RP-HPLC (water×0.1% TFA/MeCN×0.1% TFA, gradient 20% to 30%) gave XT94 (22 mg, 28%, 3 steps) as a white solid.

¹H NMR (400 MHz, DMSO-d₆) ppm=13.13-12.34 (m, 2H), 10.28 (s, 1H), 9.25 (br s, 1H), 9.16 (br s, 1H), 8.75 (s, 1H), 8.58 (br s, 1H), 8.50 (d, J=7.7 Hz, 1H), 8.42 (br s, 1H), 8.23 (d, J=6.7 Hz, 1H), 7.94 (br s, 1H), 7.84-7.76 (m, 4H), 7.67-7.61 (m, 1H), 7.49 (br s, 1H) 7.36 (d, J=8.3 Hz, 2H), 6.99 (s, 2H), 4.42-4.32 (m, 2H), 4.19-4.13 (m, 1H), 3.27-3.10 (m, 8H), 2.22-2.07 (m, 2H), 2.00-1.89 (m, 1H), 1.89-1.70 (m, 2H), 1.68-1.41 (m, 6H), 1.32 (d, J=7.1 Hz, 3H), 1.22-1.12 (m, 2H), 0.88-0.79 (m, 6H).

MS (ESI⁺) calc. for C₄₆H₅₅N₁₆O₉⁺ [M+H]⁺ 975.43, found 976.02.

### Preparation of (S)-2-(4-(2-(2,4-diaminopteridin-6-yl)ethyl)benzamido)-5-(5-((S)-2-((S)-2-(6-(2,5-dioxo-2,5-dihydro-1H-pyrrol-1-yl)hexanamido)-3-methylbutanamido)propanamido) thiophene-2-carboxamido)pentanoic acid (XT97)

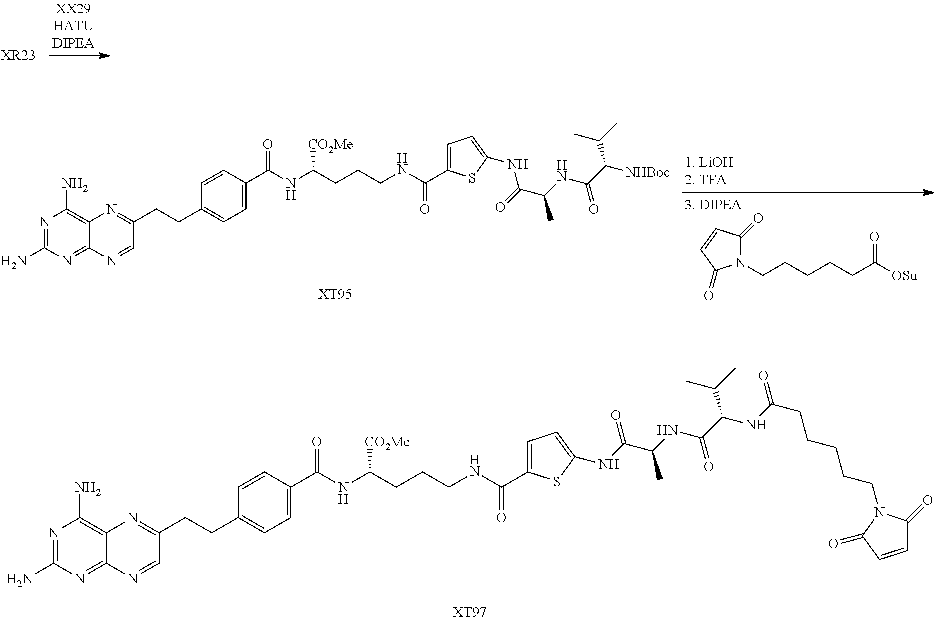

### Methyl (S)-5-(5-((S)-2-((S)-2-((tert-butoxycarbonyl)amino)-3-methylbutanamido) propanamido)thiophene-2-carboxamido)-2-(4-(2-(2,4-diaminopteridin-6-yl)ethyl) benzamido)pentanoate (XT95)

Amine XX29 (334 mg, 0.61 mmol) was reacted with acid XR23 (250 mg, 0.61 mmol) according to general procedure XXA. The product was purified by flash chromatography (silica gel, MeOH:DCM 0:1 to 1:3) to yield XT95 (quant) as a yellow solid.

MS (ESI⁺) calc. for C₃₉H₅₂N₁₁O₈⁺ [M+H]⁺ 834.37, found 834.53.

### (S)-2-(4-(2-(2,4-Diaminopteridin-6-yl)ethyl)benzamido)-5-(5-((S)-2-((S)-2-(6-(2,5-dioxo-2,5-dihydro-1H-pyrrol-1-yl)hexanamido)-3-methylbutanamido)propanamido) thiophene-2-carboxamido)pentanoic acid (XT97)

The hydrolysis and deprotection of ester XT95 was carried out according to the procedure for XT93. The crude amine was reacted with 6-maleimidohexanoic acid N-hydroxylsuccinimide ester (92 mg, 0.3 mmol) and DIPEA (388 mg, 3.00 mmol) according to the procedure for XT17. Purification by preparative RP-HPLC (water×0.1% TFA/MeCN×0.1% TFA, gradient 20% to 30%) gave XT97 (43 mg, 16%, 3 steps) as a white solid.

¹H NMR (400 MHz, DMSO-d₆) ppm=13.36-12.86 (br s, 1H (TFA)), 12.83-12.26 (br s, 1H), 11.34 (s, 1H), 9.25 (br s, 1H), 9.16 (br s, 1H), 8.75 (s, 1H), 8.60 (br s, 1H), 8.55 (d, J=7.7 Hz, 1H), 8.34-8.25 (m, 2H), 7.91-7.75 (m, 4H), 7.50 (d, J=4.1 Hz, 1H), 7.36 (d, J=8.2 Hz, 2H), 6.99 (s, 2H), 6.66 (d, J=4.1 Hz, 1H), 4.43-4.33 (m, 2H), 4.19-4.12 (m, 1H), 3.29-3.15 (m, 8H) 2.23-2.06 (m, 2H), 2.01-1.71 (m, 3H), 1.69-1.41 (m, 6H), 1.31 (d, J=7.2 Hz, 3H), 1.23-1.12 (m, 2H), 0.85 (dd, J=16.1, 6.7 Hz, 6H).

MS (ESI⁺) calc. for C₄₃H₅₃N₁₂O₉⁺ [M+H]⁺ 913.38, found 913.83.

### Preparation of (2S,2′S)-5,5′-((5,5′-(((14S,14′S,17S,17′S)-1,1′-(((((S)-2-(6-(2,5-dioxo-2,5-dihydro-1H-pyrrol-1-yl)hexanamido)pentanedioyl)bis(azanediyl))bis(methylene))bis(1H-1,2,3-triazole-4,1-diyl))bis(14-isopropyl-17-methyl-12,15-dioxo-3,6,9-trioxa-13,16-diaza octadecan-18-oyl))bis(azanediyl))bis(thiophene-5,2-diyl-2-carbonyl))bis(azanediyl))bis(2-(4-(2-(2,4-diaminopteridin-6-yl)ethyl)benzamido)pentanoic acid) (XT100)

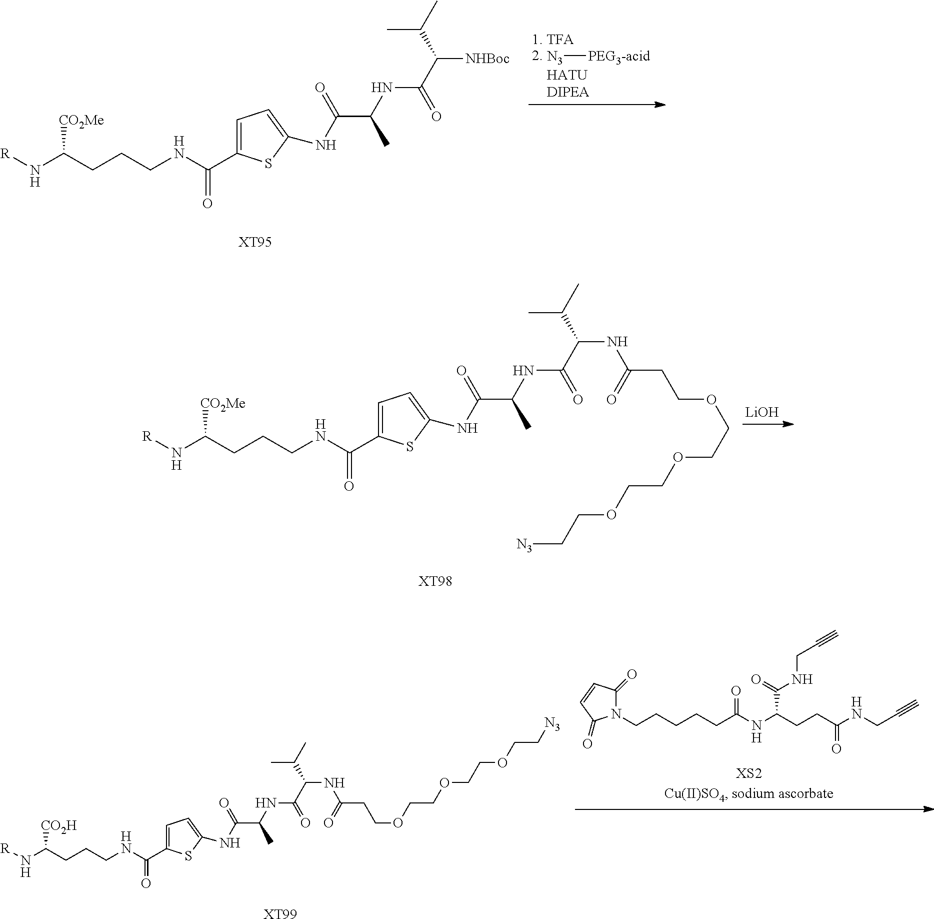

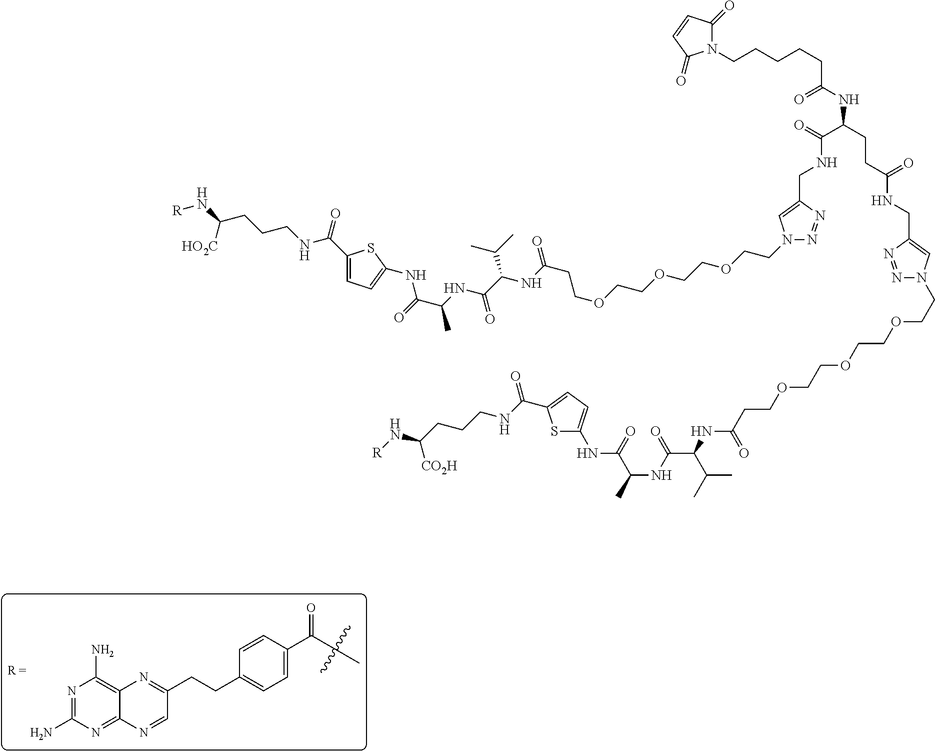

### Methyl (S)-5-(5-((14S,17S)-1-azido-14-isopropyl-17-methyl-12,15-dioxo-3,6,9-trioxa-13,16-diazaoctadecan-18-amido)thiophene-2-carboxamido)-2-(4-(2-(2,4-diaminopteridin-6-yl) ethyl)benzamido)pentanoate (XT98)

Dipeptide XT95 (250 mg, 0.30 mmol) was dissolved in DCM (1 mL), cooled to 0° C. and TFA (1 mL) was added. The resulting solution was stirred for 15 min whilst gradually warming to RT. The reaction mixture was then diluted with DCM, concentrated and coevaporated with DCM. The residue was suspended in ether (4 mL), filtered, washed with ether and dried on air to afford the intermediate amine as a TFA-salt. Next, the amine was dissolved in DMF and 3-(2-(2-(2-azidoethoxy)ethoxy)ethoxy)propanoic acid (74 mg, 0.30 mmol) was added, the mixture was cooled to 0° C. followed by addition of HATU (137 mg, 0.36 mmol) and DIPEA (0.31 mL, 1.80 mmol). The resulting mixture was stirred at RT for 1 h. The reaction mixture was subsequently concentrated and the crude product was purified by flash chromatography (silica gel, MeOH:DCM 0:1 to 1:3) to afford azide XT98 (quant) as a yellow glass.

MS (ESI⁺) calc. for C₄₃H₅₉N₁₄O₁₀⁺ [M+H]⁺ 963.43, found 963.56.

### (S)-5-(5-((14S,17S)-1-Azido-14-isopropyl-17-methyl-12,15-dioxo-3,6,9-trioxa-13,16-diazaoctadecan-18-amido)thiophene-2-carboxamido)-2-(4-(2-(2,4-diaminopteridin-6-yl) ethyl)benzamido)pentanoic acid (XT99)

Ester XT98 (290 mg, 0.30 mmol) was hydrolyzed with LiOH according to the procedure for XT93. (The Boc-deprotection step with TFA was omitted.) Purification by preparative RP-HPLC (water×0.1% TFA/MeCN×0.1% TFA, gradient 20% to 30%) afforded XT99 (27 mg, 9%) as a white solid.

¹H NMR (400 MHz, DMSO-d₆) ppm=13.08-12.39 (m, 2H (TFA)), 11.33 (s, 1H), 9.21 (br s, 1H), 9.12 (br s, 1H), 8.75 (s, 1H), 8.55 (d, J=7.7 Hz, 1H), 8.52 (br s, 1H), 8.31 (d, J=6.7 Hz, 1H), 8.28 (t, J=5.2 Hz, 1H), 7.88 (d, J=8.6 Hz, 1H), 7.82 (d, J=8.1 Hz, 2H), 7.50 (d, J=4.1 Hz, 1H), 7.36 (d, J=8.2 Hz, 2H), 6.66 (d, J=4.1 Hz, 1H), 4.43-4.34 (m, 2H), 4.23-4.17 (m, 1H), 3.63-4.45 (m, 14H), 3.27-3.14 (m, 6H), 2.49-2.35 (m, 2H), 2.01-1.70 (m, 3H), 1.68-1.51 (m, 2H), 1.31 (d, J=7.1 Hz, 3H), 0.86 (dd, J=15.9, 6.7 Hz, 6H).

MS (ESI⁺) calc. for C₄₂H₅₇N₁₄O₁₀⁺ [M+H]⁺ 949.41, found 949.88.

### (2,2'S)-5,5′-((5,5′-(((14S,14'S,17S,17'S)-1,1′-(((((S)-2-(6-(2,5-Dioxo-2,5-dihydro-1H-pyrrol-1-yl)hexanamido)pentanedioyl)bis(azanediyl))bis(methylene))bis(1H-1,2,3-triazole-4,1-diyl)) bis(14-isopropyl-17-methyl-12,15-dioxo-3,6,9-trioxa-13,16-diazaoctadecan-18-oyl)) bis(azanediyl))bis(thiophene-5,2-diyl-2-carbonyl))bis(azanediyl))bis(2-(4-(2-(2,4-diaminopteridin-6-yl)ethyl)benzamido)pentanoic acid) (XT100)

A solution of XT99 (22 mg, 0.019 mmol) and XS2 (3.1 mg, 7.5 μmol) in DMF (2.5 mL) was purged with N₂ for 5 min. Cu(II)SO₄ (1.44 mg, 5.8 μmol) in water (60 μL) and sodium ascorbate (2.22 mg, 0.011 mmol) in water (60 μL) were then added sequentially. The resulting mixture was stirred at RT for 24 h. The reaction mixture was concentrated and the crude product was purified by preparative RP-HPLC (water×0.1% TFA/MeCN×0.1% TFA, gradient 20% to 30%) to afford XT100 (12.1 mg, 70%).

MS (ESI⁺) calc. for C₁₀₅H₁₄₀N₃₂O₂₅S₂²⁺ [M+2H]²+1156.5, found [M+2H]²+1157.10.

### Preparation of (S)-5-amino-2-(N-(4-carboxy-4-(4-(((2,4-diaminopteridin-6-yl)methyl)amino) benzamido)-butyl)-sulfamoyl)benzoic acid (XT101)

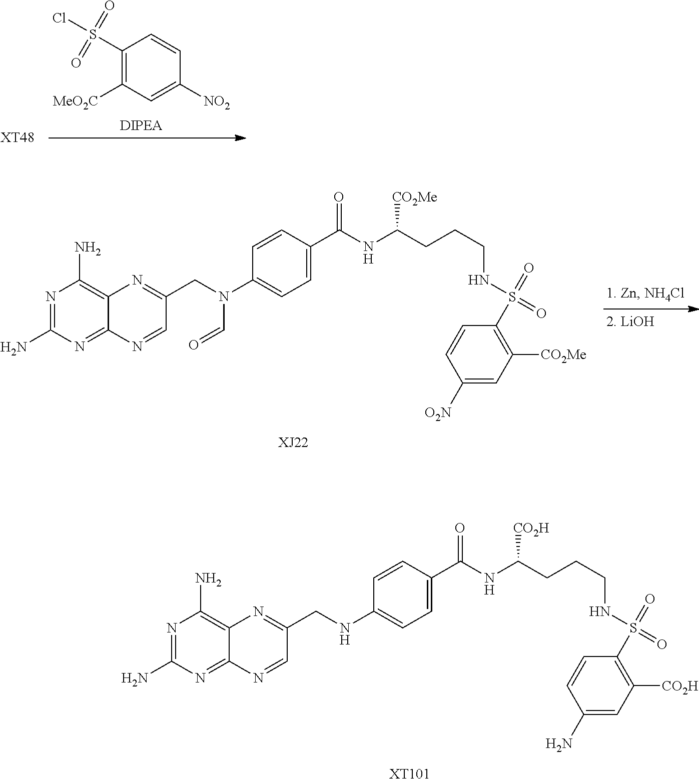

### Methyl (S)-2-(N-(4-(4-(N-((2,4-diaminopteridin-6-yl)methyl)formamido)benzamido)-5-methoxy-5-oxopentyl)sulfamoyl)-5-nitrobenzoate (XJ22)

Methyl 2-(chlorosulfonyl)-5-nitrobenzoate (0.25 g, 0.894 mmol) was reacted at RT with amine XT48 (0.416 g, 0.770 mmol) in DMF (5 mL), in the presence of DIPEA (0.33 mL, 1.92 mmol) for 0.5 h. After concentration of the reaction mixture, the residue was purified by flash chromatography (silica gel, DCM:MeOH 1:0 to 95:5) to give sulfonamide XJ22 (0.159 g, 29%) as a yellow solid.

MS (ESI⁺) calc. for C₂₉H₃₁N₁₀O₁₀S⁺ [M+H]⁺ 711.7, found 711.6.

### (S)-5-Amino-2-(N-(4-carboxy-4-(4-(((2,4-diaminopteridin-6-yl)methyl)amino)benzamido) butyl)-sulfamoyl)benzoic acid (XT101)

Aniline XT101 was prepared analogous to the preparation of XR22, starting from sulfonamide XJ22 (160 mg, 0.225 mmol). The reduction was carried out with zinc dust (442 mg, 6.75 mmol) and aq. sat. NH₄Cl (0.375 mL) in DMF (1.5 mL), followed by hydrolysis with LiOH (54 mg, 2.25 mmol) in THF (2.5 mL) and water (2.5 mL). A portion of the crude product was purified by preparative RP-HPLC (water×0.1% TFA/MeCN×0.1% TFA, gradient 5% to 35%) to give XT101 (13 mg).

¹H NMR (400 MHz, DMSO-d₆) ppm=12.46 (br s, 1H), 9.22 (br s, 1H), 9.18 (br s, 1H), 8.83 (s, 1H), 8.59-8.32 (m, 1H), 8.11 (d, J=7.6 Hz, 1H), 7.71 (d, J=8.6 Hz, 2H), 7.64-7.42 (m, 1H), 7.49 (d, J=8.8 Hz, 1H), 6.88 (br s, 1H), 6.78-6.71 (m, 3H), 6.64 (dd, J=8.7, 2.3 Hz, 1H), 6.59-6.38 (m, 1H), 6.46 (t, J=5.9 Hz, 1H), 6.13 (br s, 2H), 4.61 (s, 2H), 4.30-4.21 (m, 1H), 2.82-2.70 (m, 2H), 1.85-1.62 (m, 2H), 1.48-1.40 (m, 2H).

MS (ESI⁺) calc. for C26H₂₉N₁₀O₇S⁺ [M+H]⁺ 625.19, found 625.32.

### Preparation of (S)-4-(4-(2-(2,4-diaminopteridin-6-yl)ethyl)benzamido)-5-methoxy-5-oxopentan-1-aminium trifluoroacetate (XX29)

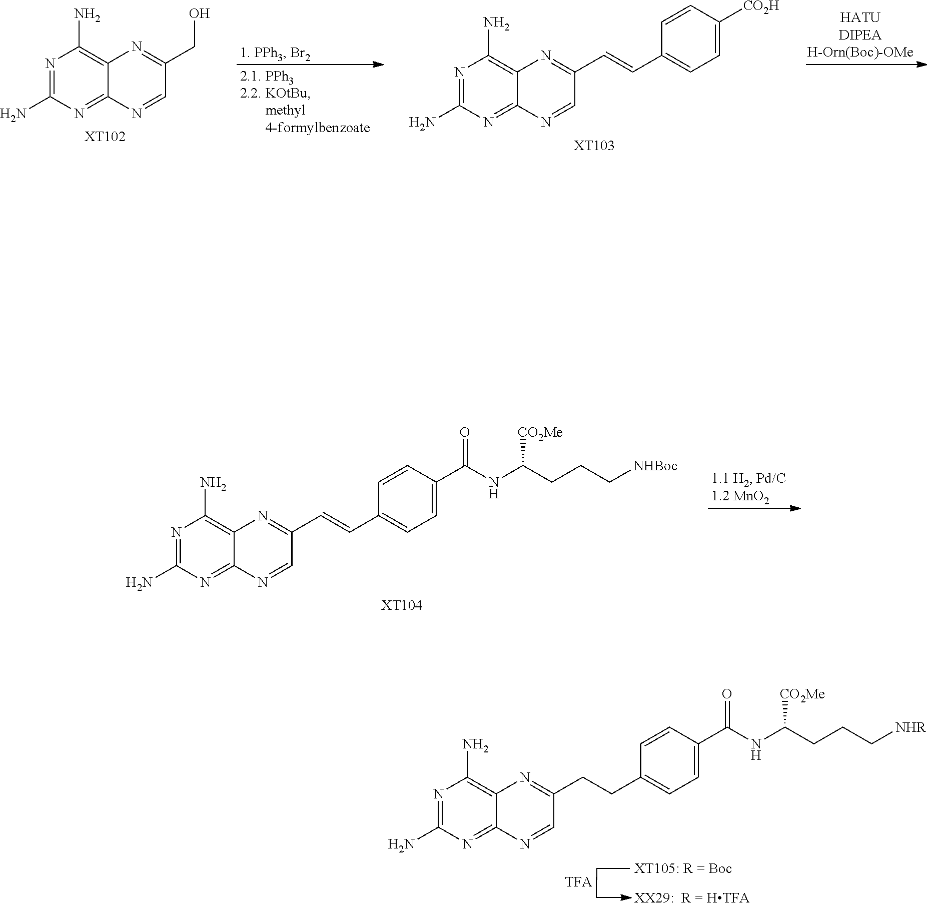

### (E)-4-(2-(2,4-Diaminopteridin-6-yl)vinyl)benzoic acid (XT103)

To a cooled (0° C.) solution of PPh₃ (31.5 g, 120 mmol) in DMA (120 mL) under N₂, was dropwise added bromine (6.17 mL, 120 mmol). Once the addition was complete, XT102 (7 g, 36.4 mmol) was added in one portion to the ice cooled solution. (The free base XT102 was prepared as described for XX9.) The resulting mixture was stirred for 1 h at 0° C., and 90 min at RT. Ethanol (2.4 mL) was added dropwise, and the mixture was stirred for 15 min at RT. Then the solution was poured into toluene (380 mL) under vigorous stirring. After filtration, the solid was stirred in ether (400 mL) until a fine suspension had formed. The suspension was filtered and the residue dried on air overnight. The resulting light-brown powder was redissolved in DMA (500 mL) under nitrogen atmosphere and PPh₃ (9.55 g, 36.4 mmol) was added to the solution. The resulting mixture was heated to 65° C. for 75 min, cooled to 0° C. and KOtBu (18.4 g, 164 mmol) was added. After stirring for 10 min at 0° C., methyl 4-formylbenzoate (5.98 g, 36.4 mmol) was added. The mixture was stirred for 45 min at RT, at which point more KOtBu (12.3 g, 109 mmol) was added. After 30 min, the reaction mixture was cooled with an ice-bath, AcOH (25 mL, 437 mmol) was added, and the mixture was stirred for 5 min. This solution was then poured into ice cold water (2.1 L). The resulting suspension was filtered and the residue was subsequently washed with MeCN (2×180 mL), toluene (2×180 mL) and ether (2×180 mL), and allowed to dry under air overnight to yield carboxylic acid XT103 (10.4 g, 93%) as a yellow solid.

¹H NMR (400 MHz, DMSO-d₆) ppm=12.96 (br s, 1H), 8.89 (s, 1H), 8.14 (br s, 1H), 8.10 (br s, 1H), 8.03-7.95 (m, 3H), 7.76 (d, J=8.2 Hz, 2H), 7.55 (d, J=16.1 Hz, 1H), 7.02 (br s, 2H).

MS (ESI⁺) calc. for C₁₅H₁₃N₆O₂⁺ [M+H]⁺ 309.11, found 309.21.

### Methyl (S,E)-5-((tert-butoxycarbonyl)amino)-2-(4-(2-(2,4-diaminopteridin-6-yl)vinyl) benzamido)pentanoate (XT104)

Acid XT103 (9.2 g, 29.8 mmol) was reacted with H-Orn(Boc)-OMe (9.3 g, 32.8 mmol) according to general procedure XXA using 10 eq of DIPEA. The crude reaction mixture was poured into water (2.4 L), stirred for 15 min, and was then filtered. The obtained solid residue was washed with ether (3×250 mL) and dried for 2 days open to air, to afford ester XT104 (1.8 g, 83%) as a yellow solid.

¹H NMR (400 MHz, DMSO-d₆) ppm=8.87 (s, 1H), 8.74 (d, J=7.4 Hz, 1H), 7.97-7.90 (m, 3H), 7.88-7.77 (m, 2H), 7.74 (d, J=8.4 Hz, 2H), 7.50 (d, J=16.2 Hz, 1H), 6.85-6.69 (m, 3H), 4.48-4.38 (m, 1H), 3.65 (s, 3H), 3.00-2.89 (m, 2H), 1.88-1.69 (m, 2H), 1.57-1.43 (m, 2H), 1.37 (s, 9H).

¹³C NMR (100 MHz, DMSO-d₆) ppm=173.2, 166.6, 163.4, 163.3, 156.0, 155.5, 150.6, 143.2, 104.1, 133.5, 131.6, 128.6, 127.1, 126.5, 122.6, 77.9, 53.0, 52.3, 39.7, 28.7, 28.3, 26.7.

MS (ESI⁺) calc. for C₂₆H₃₃N₈O₅⁺ [M+H]⁺ 537.26, found 537.67.

### Methyl (S)-5-((tert-butoxycarbonyl)amino)-2-(4-(2-(2,4-diaminopteridin-6-yl)ethyl) benzamido)pentanoate (XT105)

To a nitrogen purged flask containing a solution of XT104 (11.2 g, 20.9 mmol) in AcOH (450 mL) was added Pd/C (5 g, 10% on activated carbon). Hydrogen gas was introduced and the mixture was stirred under hydrogen atmosphere at RT. Three extra portions of Pd/C (2.5 g) were added with 3 h intervals. After 24 h, the flask was purged with N₂, and the reaction mixture was then filtered over Celite. MnO₂ (18.2 g, 209 mmol) was added to the filtrate and the suspension was stirred for 30 min. After filtration over Celite, the filtrate was concentrated, and the crude product was purified by flash chromatography (silica gel, MeOH:DCM 0:1 to 15:85) to afford XT105 (6.9 g, 61%) as a sand colored solid.

¹H NMR (400 MHz, DMSO-d₆) ppm=8.61 (d, J=7.3 Hz, 1H), 8.55 (s, 1H), 7.79 (d, J=8.3 Hz, 2H), 7.59 (br s, 2H), 7.35 (d, J=8.3 Hz, 2H), 6.77 (t, J=5.4 Hz, 1H), 6.54 (br s, 2H), 4.43-4.33 (m, 1H), 3.63 (s, 3H), 3.19-3.09 (m, 4H), 2.97-2.88 (m, 2H), 1.86-1.67 (m, 2H), 1.55-1.39 (m, 2H), 1.36 (s, 9H).

MS (ESI⁺) calc. for C₂₆H₃₅N₈O₅⁺ [M+H]⁺ 539.27, found 539.62.

### (S)-4-(4-(2-(2,4-Diaminopteridin-6-yl)ethyl)benzamido)-5-methoxy-5-oxopentan-1-aminium trifluoroacetate (XX29)

Compound XT105 (1.10 g, 2.04 mmol) was suspended in DCM (10 mL) and TFA (10 mL) was added at RT. After stirring for 20 min at RT the mixture was concentrated. The residue was coevaporated with iPrOH (2×), and was subsequently suspended in iPrOH (10 mL). The suspension was diluted with ether (60 mL) and the solid was filtered off and dried under vacuum, to give TFA-salt XX29 (1.03 g, 88%) as a yellow solid.

¹H NMR (400 MHz, DMSO-d₆) ppm=9.24 (s, 1H), 9.13 (s, 1H), 8.73 (s, 1H), 8.72 (s, 1H), 8.54 (br s, 1H), 8.23 (br s, 1H), 7.82 (d, J=8.2 Hz, 2H), 7.78 (br s, 3H), 7.37 (d, J=8.2 Hz, 2H), 4.46 (ddd, J=9.5, 7.7, 5.1 Hz, 1H), 3.65 (s, 3H), 3.28-3.19 (m, 4H), 2.85-2.77 (m, 2H), 1.96-1.74 (m, 2H), 1.72-1.56 (m, 2H).

MS (ESI⁺) calc. for C₂₁H₂₇N₈O₃⁺ [M+H]⁺ 439.22, found 439.56.

### Preparation of 2,5-dioxopyrrolidin-1-yl (6-(2,5-dioxo-2,5-dihydro-1H-pyrrol-1-yl)hexanoyl) glycylglycyl-L-phenylalaninate (XX30)

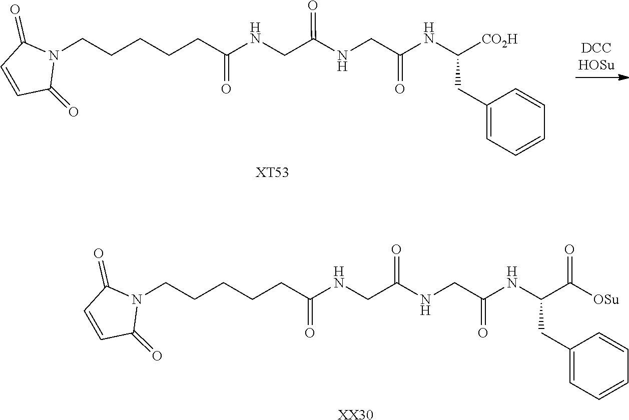

DCC (459 mg, 2.222 mmol) was added to a suspension of XT53 (1.05 mg, 2.22 mmol) and 1-hydroxypyrrolidine-2,5-dione (256 mg, 2.222 mmol) in THF (40 mL) at RT. After stirring for 3.5 h, the mixture was filtered and DCM was used to wash the residue thoroughly. The filtrate was diluted with EtOAc and was then concentrated. The white solid was suspended in a small volume of EtOAc and was then filtered to give OSu-ester XX30 (612 mg, 48%) as a white solid.

MS (ESI⁺) calc. for C₂₇H₃₂N₅O₉⁺ [M+H]⁺ 570.22, found 570.43.

### Preparation of sodium 5-((S)-5-benzyl-18-(2,5-dioxo-2,5-dihydro-1H-pyrrol-1-yl)-4,7,10,13-tetraoxo-3,6,9,12-tetraazaoctadecanamido)-2-(((S)-4-carboxy-4-(4-(((2,4-diaminopteridin-6-yl)methyl)amino)benzamido)butyl)carbamoyl)benzenesulfonate (XX19)

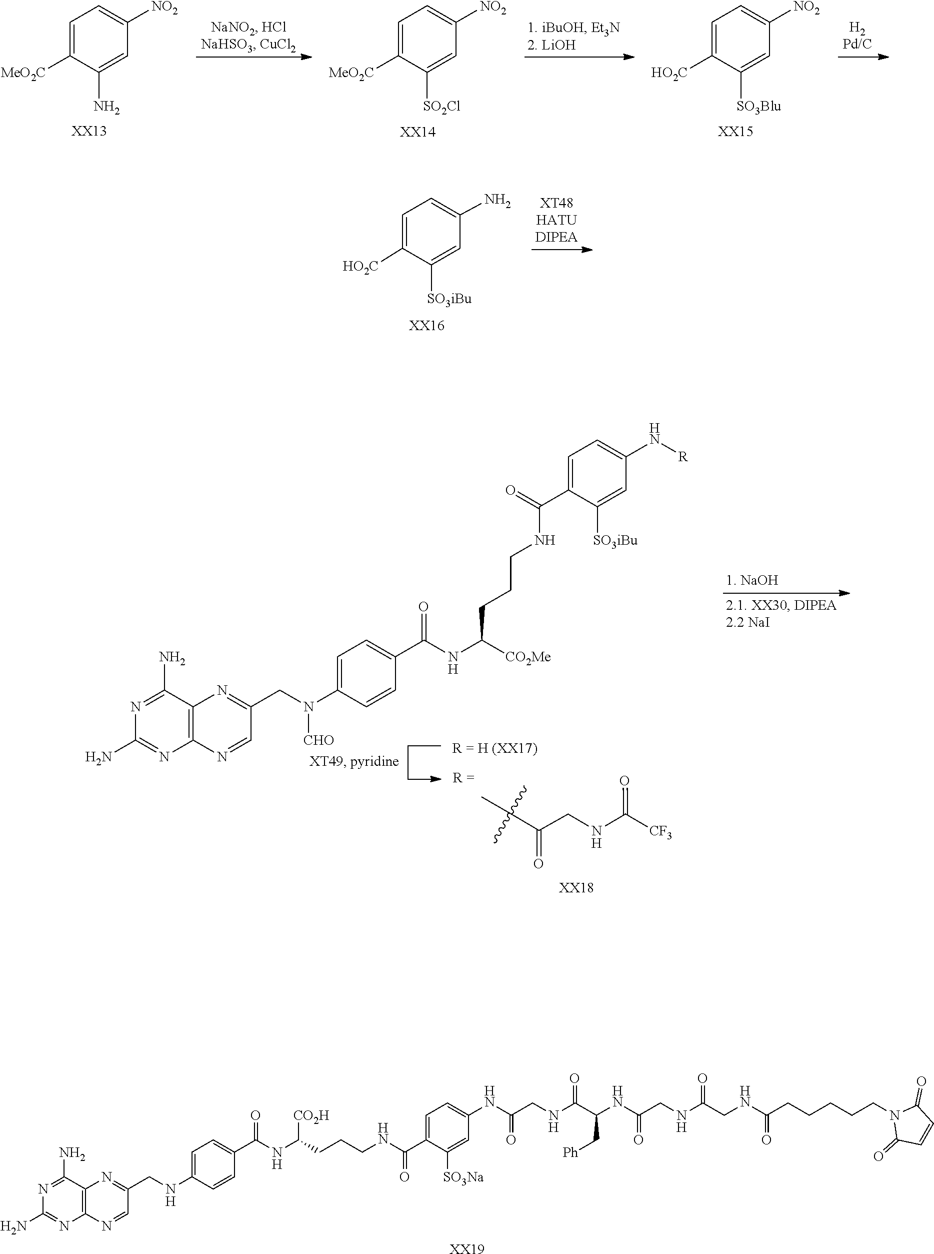

### Methyl 2-(chlorosulfonyl)-4-nitrobenzoate (XX14)

To aniline XX13 (2.47 g, 12.6 mmol) in EtOAc (21 mL) and conc. HCl (22 mL) was added dropwise a solution of NaNO₂ (0.912 g, 13.2 mmol) in water (5.7 mL) at 0° C. After stirring for 30 min at 0° C., a suspension of Cu(II)Cl₂ dihydrate (0.966 g, 5.67 mmol), NaHSO₃ (13.1 g, 126 mmol), AcOH (16 mL) and conc. HCl (5.7 mL) was added, and the reaction was stirred at RT for 30 min. The reaction mixture was poured onto ice, and the precipitated sulfonyl chloride was filtered off and washed with water. The solid was dried under vacuum to give the product XX14 (2.58 g, 73%) as a yellow solid.

¹H NMR (400 MHz, CDCl₃) ppm=8.99 (d, J=2.0 Hz, 1H), 8.64-8.62 (dd, J=8.3 Hz, 2.1 Hz, 1H), 7.95 (d, J=8.4 Hz, 1H), 4.05 (s, 3H).

¹³C NMR (100 MHz, CDCl₃) ppm=164.6, 148.7, 142.9, 137.3, 131.6, 129.5, 124.5, 54.0.

### 2-(Isobutoxysulfonyl)-4-nitrobenzoic acid (XX15)

Sulfonyl chloride XX14 (0.971 g, 3.47 mmol) was dissolved in DCM (20 mL) and the yellow solution was cooled to 0° C. Isobutanol (1.61 mL, 17.4 mmol) and Et₃N (0.726 mL, 5.21 mmol) were added and the reaction was stirred for 1 h at 0° C. The solution was concentrated, dissolved in EtOAc and washed with KHSO₄ (0.5 M, 2×), sat. NaHCO₃ and brine. The organic layer was dried on MgSO₄, filtered and concentrated. Purification by flash chromatography (silica gel, heptane:DCM 1:0 to 0:1) afforded the product XX15 (911 mg, 83%) as a pale yellow oil. A portion of this material (791 mg, 2.49 mmol) was dissolved in dioxane (25 mL) and water (16 mL). LiOH (2M, 4.99 mL, 9.97 mmol) was added at RT. After 20 min, the reaction was diluted with water and acidified to pH 3 with 1 M HCl. Extracted with EtOAc (3×). The combined organic layers were washed with brine, dried over Na₂SO₄, filtered and concentrated to give acid XX15 (756 mg, quant) as a yellow oil.

¹H NMR (400 MHz, CDCl₃) ppm=8.89 (d, J=2.1 Hz, 1H), 8.55 (dd, J=8.4, 2.1 Hz, 1H), 8.0 (d, J=8.4 Hz, 1H), 4.05 (d, J=6.4 Hz, 2H), 2.05 (sept, J=6.7 Hz, 1H), 0.96 (d, J=6.7 Hz, 6H).

MS (ESI⁻) calc. for C₁₁H₁₂NO₇S⁻ [M−H]⁻ 302.03, found 302.35.

### 4-Amino-2-(isobutoxysulfonyl)benzoic acid (XX16)

Palladium (42 mg, 10% on activated carbon) was added to acid XX15 (104 mg, 0.342 mmol) in MeOH (4 mL) at RT under N₂ atmosphere. The mixture was then stirred under hydrogen atmosphere for 30 min. The flask was purged with N₂, and the mixture was filtered over Celite. Concentration then afforded aniline XX16 (83 mg, 89%) as a white solid.

MS (ESI⁻) calc. for C₁₁H₁₄NO₅S⁻ [M−H]⁻ 272.06, found 272.34.

### Methyl (S)-5-(4-amino-2-(isobutoxysulfonyl)benzamido)-2-(4-(N-((2,4-diaminopteridin-6-yl)methyl)formamido)benzamido)pentanoate (XX17)

Acid XX16 (83 mg, 0.304 mmol) was reacted with HATU (121 mg, 0.319 mmol) and amine XT48 (153 mg, 0.304 mmol) according to general procedure XXA. After concentration of the reaction mixture, the residue was coevaporated with MeCN (2×), resuspended in MeCN and filtered. The solid was purified by flash chromatography (silica gel, DCM:MeOH 1:0 to 4:1) to give aniline XX17 (136 mg, 62%) as a yellow solid.

¹H NMR (400 MHz, DMSO-d₆) ppm=8.80 (s, 1H), 8.70 (d, J=7.4 Hz, 1H), 8.66 (s, 1H), 8.03 (t, J 5.4 Hz, 1H), 7.90 (d, J=8.7 Hz, 2H), 7.74 (br s, 1H), 7.59 (d, J=8.7 Hz, 2H), 7.43 (br s, 1H), 7.14 (d, J=8.3 Hz, 1H), 7.10 (d, J=2.2 Hz, 1H), 6.80 (dd, J=8.1, 2.2 Hz, 1H), 6.70 (br s, 2H), 5.88 (br s, 2H), 5.22 (s, 2H), 4.44-4.37 (m, 1H), 3.83 (d, J=6.4 Hz, 2H), 3.63 (s, 3H), 3.19-3.13 (m, 2H), 1.93-1.75 (m, 3H), 1.65-1.48 (m, 2H), 0.80 (d, 6.7 Hz, 6H).

MS (ESI⁺) calc. for C₃₂H₃₉N₁₀O₈S⁺ [M+H]⁺ 723.27, found 723.45.

### Methyl (S)-2-(4-(N-((2,4-diaminopteridin-6-yl)methyl)formamido)benzamido)-5-(2-(isobutoxysulfonyl)-4-(2-(2,2,2-trifluoroacetamido)acetamido)benzamido)pentanoate (XX18)

A stock solution of crude XT49 (205 mg, 1.08 mmol) in THF (0.5 mL) was prepared under N₂. In a separate vial aniline XX17 (130 mg, 0.180 mmol) and DIPEA (0.094 mL, 0.540 mmol) were dissolved in DMF (2 mL) under N₂. A portion of the stock solution (285 μL) was added to the aniline at RT. After 5 min, a second portion (285 μL) was added and stirring was continued for 5 min. The reaction was concentrated and dryloaded on silica gel. Purification by flash chromatography (silica gel, DCM:MeOH 1:0 to 4:1) afforded amide XX18 (106 mg, 67%).

¹H NMR (400 MHz, DMSO-d₆) ppm=10.67 (s, 1H), 9.83 (t, J=5.8 Hz, 1H), 8.80 (s, 1H), 8.70 (d, J=7.5 Hz, 1H), 8.65 (s, 1H), 8.34 (t, J=5.5 Hz, 1H), 8.24 (d, J=2.0 Hz, 1H), 7.89 (d, J=8.8 Hz, 2H), 7.87 (dd, J=8.6, 6.6 Hz, 1H), 7.64 (br s, 1H), 7.59 (d, J=8.5 Hz, 2H), 7.48 (d, J=8.3 Hz, 1H), 7.33 (br s, 1H), 6.61 (br s, 2H), 5.22 (s, 2H), 4.45-4.38 (m, 1H), 4.06 (d, J=5.6 Hz, 2H), 3.87 (d, J=6.5 Hz, 2H), 3.63 (s, 3H), 3.20 (q, J=6.0 Hz, 2H), 1.94-1.75 (m, 3H), 1.66-1.545 (m, 2H), 0.79 (d, J=6.6 Hz, 6H).

MS (ESI⁺) calc. for C₃₆H₄₁F₃N₁₁O₁₀S⁺ [M+H]⁺ 876.27, found 876.21.

### Sodium 5-((S)-5-benzyl-18-(2,5-dioxo-2,5-dihydro-1H-pyrrol-1-yl)-4,7,10,13-tetraoxo-3,6,9,12-tetraazaoctadecanamido)-2-(((S)-4-carboxy-4-(4-(((2,4-diaminopteridin-6-yl) methyl)amino)benzamido)butyl)carbamoyl)benzenesulfonate (XX19)

Ester XX18 (79.2 mg, 0.090 mmol) was hydrolyzed according to general procedure XXC with the following modifications: i) the reaction was not warmed to RT, but kept at 0° C., ii) NaOH (12 eq) was added in two portions with a 20-min interval, iii) washing of the solid with MeOH and ether was omitted. Drying of the solid under vacuum overnight afforded the crude acid (68 mg). Activated ester XX30 (52.5 mg, 0.092 mmol) in DMF (1.0 mL) was then added to the yellow solid at RT, followed by DIPEA (0.097 mL, 0.553 mmol). After stirring for 20 min, more XX30 (10.5 mg, 0.018 mmol) was added. Stirring was continued for 2 min at which point the reaction was concentrated. The residue was suspended in MeCN (5 mL) and stirred for 1 h. The suspension was filtered and the solid was washed with MeCN and ether. The yellow solid (107 mg) was suspended in acetone (11 mL) and NaI (207 mg, 1.384 mmol) was added. The vial was capped and heated at 60° C. for 90 min. After cooling to RT, the suspension was filtered and the solid washed with acetone (1 mL). A portion of the crude product was purified by preparative RP-HPLC (water×0.10% TFA/MeCN×0.1% TFA, gradient 20% to 50%) to give XX19 (4.8 mg) as a yellow solid.

MS (ESI⁺) calc. for C₅₁H₅₈N₁₅O₁₄S⁺ [M+H]⁺ 1136.40, found 1136.95.

### Preparation of (S)-5-(4-amino-2-sulfobenzamido)-2-(4-(((2,4-diaminopteridin-6-yl)methyl) amino)benzamido)pentanoic acid (XX37)

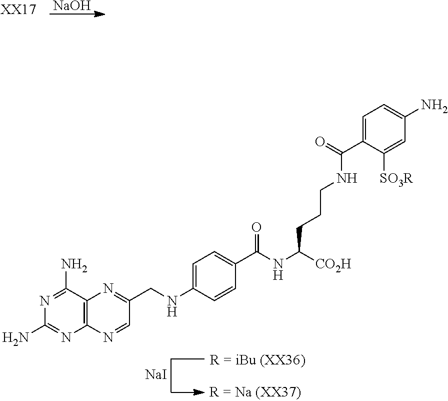

### (S)-5-(4-Amino-2-(isobutoxysulfonyl)benzamido)-2-(4-(((2,4-diaminopteridin-6-yl)methyl) amino)-benzamido)pentanoic acid (XX36)

The hydrolysis of crude ester XX17 (128 mg, 0.177 mmol) was carried out according to general procedure XXC to give XX36 as a yellow solid.

MS (ESI⁺) calc. for C₃₀H₃₇N₁₀O₇S⁺ [M+H]⁺ 681.26, found 681.46.

### (S)-5-(4-Amino-2-sulfobenzamido)-2-(4-(((2,4-diaminopteridin-6-yl)methyl)amino) benzamido)pentanoic acid (XX37)

Alkyl sulfonate XX36 (35.7 mg, 0.052 mmol) and NaI (118 mg, 0.787 mmol) in acetone (6 mL) were heated in a sealed vial at 60° C. for 8 h. The reaction was concentrated and a portion of the crude product was purified by preparative RP-HPLC (water×0.1% TFA/MeCN×0.1% TFA, gradient 5% to 20%) to give XX37 (4.0 mg) as a yellow solid.

¹H NMR (400 MHz, DMSO-d₆) ppm=12.24 (br s, 1H), 9.28 (s, 1H), 9.22 (s, 1H), 9.20 (t, J=5.5 Hz, 1H), 8.77 (s, 1H), 8.55 (br s, 1H), 8.07 (d, J=7.1 Hz, 1H), 7.73 (d, J=8.8 Hz, 2H), 7.50 (br s, 1H), 7.34 (d, J=8.4 Hz, 2H), 7.08 (d, J=2.4 Hz, 1H), 6.80 (br s, 1H), 6.65 (d, J=8.8 Hz, 2H), 6.46 (dd, J=8.3, 2.2 Hz, 2H), 4.55 (s, 2H), 4.17-4.12 (m, 1H), 3.22-3.06 (m, 2H), 1.97-1.84 (m, 1H), 1.78-1.70 (m, 1H), 1.59-1.42 (m, 2H).

MS (ESI⁺) calc. for C26H₂₉N₁₀O₇S⁺ [M+H]⁺ 625.19, found 625.20.

### Preparation of (S)-5-(4-Amino-2-(2H-tetrazol-5-yl)benzamido)-2-(4-(((2,4-diaminopteridin-6-yl)methyl)amino)benzamido)pentanoic acid (XX35)

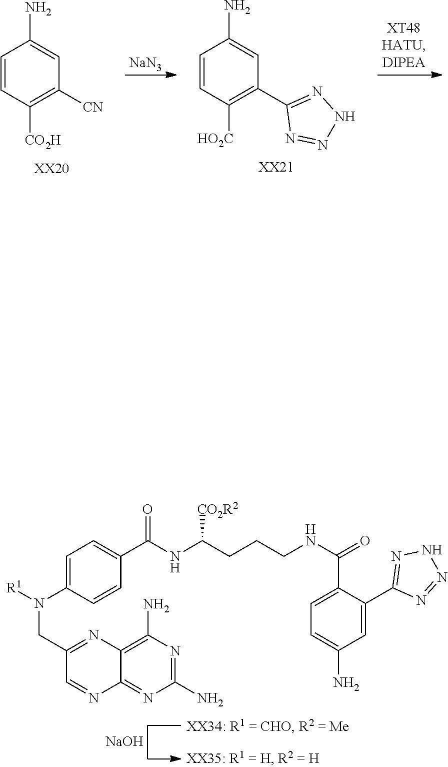

### 4-Amino-2-(2H-tetrazol-5-yl)benzoic acid (XX21)

A solution of 4-amino-2-cyanobenzoic acid (XX20; 250 mg, 1.542 mmol) and NaN₃ (203 mg, 3.13 mmol) in DMF (4 mL) was heated at 110° C. for 16 h in a sealed vial under N₂. After cooling to RT, the solution was filtered and the filtrate was used directly in the next step.

MS (ESI⁺) calc. for C₈H₈N₅O₂⁺ [M+H]⁺ 206.07, found 206.22.

### Methyl (S)-5-(4-amino-2-(2H-tetrazol-5-yl)benzamido)-2-(4-(N-((2,4-diaminopteridin-6-yl) methyl)formamido)benzamido)pentanoate (XX34)

An aliquot of XX21 (0.5 mL crude filtrate in DMF) was added to a solution of XT48 (100 mg, 0.185 mmol) and DIPEA (0.194 mL, 1.110 mmol) in DMF (1.0 mL). HATU (70.4 mg, 0.185 mmol) was added at RT and the resulting mixture was stirred for 30 min. More XT48 (20 mg, 0.037 mmol) and HATU (14.07 mg, 0.037 mmol) was added, and after 15 min the reaction was concentrated and suspended in MeCN (6 mL). After stirring for 45 min, the suspension was filtered and the solid was washed with MeCN and ether to give crude XX34 (126 mg) as a cream solid.

MS (ESI⁺) calc. for C₂₉H₃₁N₁₄O₅⁺ [M+H]⁺ 655.26, found 655.42.

### (S)-5-(4-Amino-2-(2H-tetrazol-5-yl)benzamido)-2-(4-(((2,4-diaminopteridin-6-yl)methyl) amino)benzamido)pentanoic acid (XX35)

The hydrolysis of crude ester XX34 (58 mg, 0.089 mmol) was carried out according to general procedure XXC. Purification of the crude product by preparative RP-HPLC (water×0.1% TFA/MeCN×0.1% TFA, gradient 10% to 25%) afforded XX35 (14.8 mg) as a yellow solid.

¹H NMR (400 MHz, DMSO-d₆) ppm=12.40 (br s, 1H), 9.24 (br s, 1H), 9.20 (br s, 1H), 8.77 (s, 1H), 8.50 (br s, 1H), 8.11-8.06 (m, 2H), 7.69-7.62 (m, 3H), 7.40-7.36 (m, 1H), 6.84 (br s, 1H), 6.67 (d, J=8.9 Hz, 2H), 6.65-6.62 (m, 2H), 4.55 (s, 2H), 4.28-4.22 (m, 1H), 3.04 (q, J=6.4 Hz, 2H), 1.80-1.60 (m, 2H), 1.53-1.36 (m, 2H).

MS (ESI⁺) calc. for C₂₇H₂₉N₁₄O₄⁺ [M+H]⁺ 613.25, found 613.34.

### Preparation of (S)-2-(4-(((2,4-Diaminopteridin-6-yl)methyl)amino)benzamido)-5-(4-((S)-2-((S)-2-(6-(2,5-dioxo-2,5-dihydro-1H-pyrrol-1-yl)hexanamido)-3-methylbutanamido) propanamido)-2-(2H-tetrazol-5-yl)benzamido)pentanoic acid (XX23)

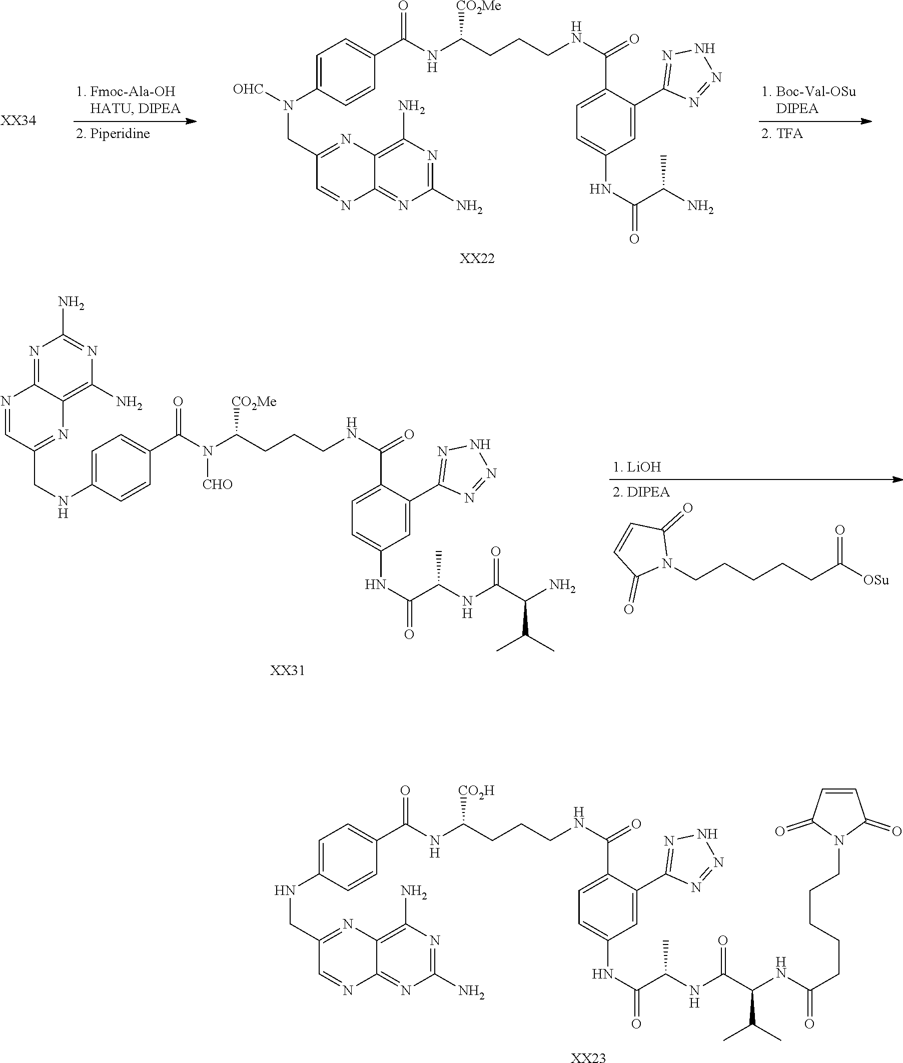

### Methyl (S)-5-(4-((S)-2-aminopropanamido)-2-(2H-tetrazol-5-yl)benzamido)-2-(4-(N-((2,4-diaminopteridin-6-yl)methyl)formamido)benzamido)pentanoate (XX22)

To a solution of crude XX34 (245 mg, 0.374 mmol) in DMF (1.8 mL) was added Fmoc-Ala-OH (175 mg, 0.561 mmol), HATU (213 mg, 0.561 mmol) and DIPEA (0.392 mL, 2.25 mmol) at RT under N₂. After 30 min, more Fmoc-Ala-OH (23.3 mg, 0.075 mmol) and HATU (28.5 mg, 0.075 mmol) was added and stirring was continued for 30 min. The reaction was concentrated, suspended in MeCN (6 mL) and stirred for 45 min at RT. After filtration, the yellow solid (355 mg) was dissolved in DMF (6 mL) and piperidine (0.593 mL, 5.99 mmol) was added at RT. After stirring for 5 min, the reaction was concentrated, suspended in ether (10 mL) and stirred for 1 h at RT. The solid was filtered off, washed with ether and dried under vacuum to give crude amine XX22 (270 mg) as a pale yellow solid.

MS (ESI⁺) calc. for C₃₂H₃₆N₁₅O₆⁺ [M+H]⁺ 726.30, found 726.27.

### Methyl (S)-5-(4-((S)-2-aminopropanamido)-2-(2H-tetrazol-5-yl)benzamido)-2-(4-(N-((2,4-diaminopteridin-6-yl)methyl)formamido)benzamido)pentanoate (XX31)

A portion of crude amine XX22 (169 mg) was dissolved in DMF (0.5 mL). Boc-Val-OSu (72.1 mg, 0.229 mmol) and DIPEA (0.073 mL, 0.417 mmol) were added at RT. After 2 h, more Boc-Val-OSu (10 mg, 0.032 mmol) and DIPEA (0.025 mL, 0.146 mmol) was added at RT and the mixture was stirred overnight. After concentration, the residue was suspended in MeCN (275 mL) and heated to reflux. The suspension was filtered hot and was then allowed to cool to RT. The solution was concentrated to approximately 15 mL volume and was then filtered to give the crude dipeptide (59 mg) as a cream solid. The solid was suspended in DCM (2 mL) and TFA (2 mL) was added at RT. After stirring for 10 min, the reaction was concentrated and purified by preparative RP-HPLC (water×0.10% TFA/MeCN×0.10% TFA, gradient 10% to 25%). MeCN was removed from product fractions by rotary evaporation and the aqueous phase was lyophilized to afford amine XX31 (22.2 mg) as a white solid.

MS (ESI⁺) calc. for C₃₇H₄₅N₁₆O₇⁺ [M+H]⁺ 825.37, found 825.50.

### (S)-2-(4-(((2,4-Diaminopteridin-6-yl)methyl)amino)benzamido)-5-(4-((S)-2-((S)-2-(6-(2,5-dioxo-2,5-dihydro-1H-pyrrol-1-yl)hexanamido)-3-methylbutanamido)propanamido)-2-(2H-tetrazol-5-yl)benzamido)pentanoic acid (XX23)

Amine XX31 (22.2 mg, 0.021 mmol) was dissolved in THF (0.6 mL)/water (0.15 mL). Lithium hydroxide hydrate (6.19 mg, 0.148 mmol) was added at RT and the mixture was stirred for 90 min. After cooling to 0° C., AcOH (0.014 mL, 0.253 mmol) was added followed by toluene (5 mL) and the mixture was concentrated. The residue was redissolved in DMF (0.5 mL), and 6-maleimidohexanoic acid N-hydroxylsuccinimide ester (6.8 mg, 0.022 mmol) and DIPEA (0.015 mL, 0.084 mmol) were added at RT. The mixture was stirred for 90 min at RT and was then concentrated. The residue was purified by preparative RP-HPLC (water×0.1% TFA/MeCN×0.1% TFA, gradient 20 to 50%). MeCN was removed from product fractions by rotary evaporation and the aqueous phase was lyophilized to afford tetrazole XX23 (9.9 mg) as a yellow solid.

MS (ESI⁺) calc. for C₄₅H₅₄N₁₇O₉⁺ [M+H]⁺ 976.43, found 976.48.

### Preparation of (S)-5-(4-(4-amino-2-cyanophenyl)-1H-1,2,3-triazol-1-yl)-2-(4-(2-(2,4-diaminopteridin-6-yl)ethyl)benzamido)pentanoic acid (XX41)

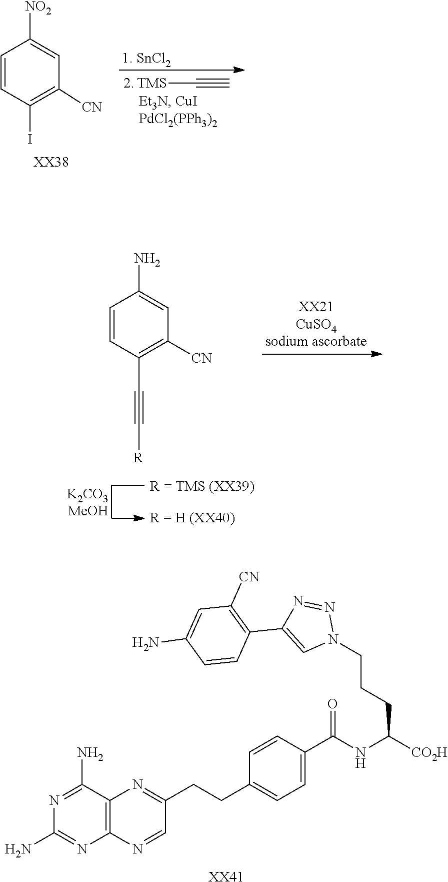

### 5-Amino-2-((trimethylsilyl)ethynyl)benzonitrile (XX39)

To a suspension of iodide XX38 (5.13 g, 18.7 mmol, prepared according to: Ozaki et al, Tetrahedron, 2017, 73, 7177-7184) in EtOH (40 mL) was added SnCl₂ dihydrate (21.1 g, 93.5 mmol) at RT under N₂. After 20 min, the suspension had turned into a dark orange red solution. The flask was cooled with a RT water bath to dissipate some of the exotherm that had developed over time. After stirring for 2 h, a new suspension had formed. The reaction was then poured into a cooled (0° C.) solution of NaOH (15.0 g) in water (120 mL). The mixture was stirred for 5 min and was then filtered. The solid was washed with cold aq. NaOH (2 M), and water. After drying under vacuum the aniline (3.94 g, 86%) was obtained as a pale yellow solid.

The aniline (3.94 g, 16.2 mmol) was loaded in a 50 mL three-neck flask and CuI (61.5 mg, 0.323 mmol) and PdCl₂(PPh₃)₂ (227 mg, 0.323 mmol) were added. The flask was purged with N₂, Et₃N (17.7 mL, 127 mmol) and ethynyltrimethylsilane (2.457 mL, 17.76 mmol) were added at RT, and the suspension was vigorously stirred for 4 h. The suspension slowly dissolved during this time. The reaction was then concentrated and purified by flash chromatography (silica gel, heptane:DCM 1:0 to 0:1) Upon concentration of product fractions, white flakes precipitated. More heptane (30 mL) was added at this point and the suspension was concentrated to a volume of ˜35 mL. The solid was filtered off and dried under vacuum to give alkyne XX39 (3.06 g, 76% 2 steps) as off-white flakes.

MS (ESI⁺) calc. for C₁₂H₁₅N₂Si⁺ [M+H]⁺ 215.10, found 215.15.

### 5-Amino-2-ethynylbenzonitrile (XX40)

A solution of XX39 (2.45 g, 11.43 mmol) in MeOH (50 mL) was treated with K₂CO₃ (3.16 g, 22.9 mmol) at RT for 15 min. The reaction was concentrated and taken up in EtOAc and water. The layers were separated and the water layer was extracted with EtOAc (lx). The combined organic layers were washed with brine, dried over Na₂SO₄, filtered and concentrated to give alkyne XX40 (1.64 g, quant) as a cream solid.

¹H NMR (400 MHz, CDCl₃) ppm=7.37 (d, J=8.5 Hz, 1H), 6.87 (d, J=2.5 Hz, 1H), 6.78 (dd, J=8.5, 2.5 Hz, 1H), 4.04 (br s, 2H), 3.30 ppm (s, 1H).

¹³C NMR (100 MHz, CDCl₃) ppm=147.0, 134.2, 118.4, 117.7, 117.5, 116.8, 114.9, 80.9, 80.2.

MS (ESI⁺) calc. for C₉H₇N₂⁺ [M+H]⁺ 143.06, found 143.05.

### (S)-5-(4-(4-Amino-2-cyanophenyl)-1H-1,2,3-triazol-1-yl)-2-(4-(2-(2,4-diaminopteridin-6-yl) ethyl)benzamido)pentanoic acid (XX41)

A solution of alkyne XX40 (29.7 mg, 0.209 mmol) and azide XS21 (94 mg, 0.209 mmol) in DMF (1.7 mL) was purged with N₂ for 5 min. CuSO₄·5H₂O (39.1 mg, 0.157 mmol) in water (1.1 mL) and sodium ascorbate (62.0 mg, 0.313 mmol) in water (1.1 mL) were added sequentially, and the resulting mixture was stirred at RT for 40 h. More DMF (1 mL) was added followed by CuSO₄·5H₂O (19.54 mg, 0.078 mmol) in water (0.5 mL) and sodium ascorbate (31.0 mg, 0.157 mmol) in water (0.5 mL) was added and the reaction was stirred at RT for an extra 3 days. The reaction was then poured into water (25 mL) and aq. AcOH (1 M, 1 mL) was added. The mixture was heated with a heatgun to break up the fine suspension. After cooling to RT, the suspension could be filtered and the solid was washed with MeCN (1 mL). A portion of the solid was purified by RP-HPLC (water×0.1% TFA/MeCN×0.1% TFA, gradient 10% to 35%), to give acid XX41 as a yellow solid.

¹H NMR (400 MHz, DMSO-d₆) ppm=12.53 (br s, 1H), 9.09 (s, 1H), 9.00 (s, 1H), 8.67 (s, 1H), 8.50 (d, J=7.8H, 1H), 8.40 (br s, 1H), 8.33 (s, 1H), 7.74 (d, J=8.3 Hz, 2H), 7.60 (d, J=8.9 Hz, 1H), 7.59 (br s, 1H), 7.28 (d, J=8.3 Hz, 2H), 6.87 (s, 1H), 6.86 (dd, J=9.0, 2.4 Hz, 1H), 4.39 (t, J=6.8 Hz, 2H), 4.38-4.32 (m, 1H), 3.20-3.11 (m, 4H), 1.96-1.83 (m, 2H), 1.80-1.66 (m, 2H).

MS (ESI⁺) calc. for C₂₉H₂₉N₁₂O₃⁺ [M+H]⁺ 593.25, found 593.49.

### Preparation of (S)-5-(4-(4-Amino-2-cyanophenyl)-1H-1,2,3-triazol-1-yl)-2-(4-(((2,4-diaminopteridin-6-yl)methyl)amino)benzamido)pentanoic acid (XX47)

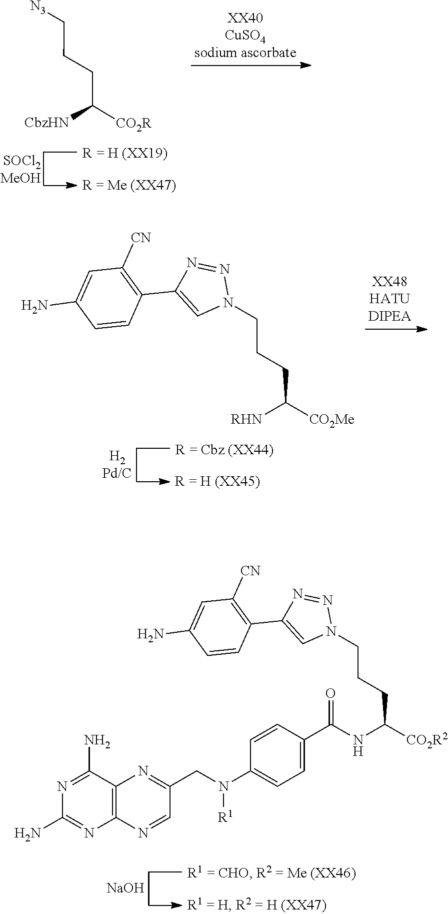

### Methyl (S)-5-azido-2-(((benzyloxy)carbonyl)amino)pentanoate (XX43)

SOCl₂ (1.73 mL, 23.9 mmol) was added to azide XR19 (3.49 g, 11.9 mmol) in MeOH (50 mL) at 0° C. The solution was heated to 60° C. for 15 min. After cooling to RT, the mixture was concentrated and the residue was coevaporated with toluene (2×). Purification by flash chromatography (silica gel, heptane:EtOAc 1:0 to 2:1) afforded methyl ester XX43 (3.52 g, 96%) as a colorless oil.

¹H NMR (400 MHz, CDCl₃) ppm=7.39-7.29 (m, 5H), 5.32 (br d, J=7.3 Hz, 1H), 5.11 (s, 2H), 4.45-4.37 (m, 1H), 3.76 (s, 3H), 3.35-3.27 (m, 2H), 2.00-1.90 (m, 1H), 1.78-1.54 (m, 3H).

MS (ESI⁺) calc. for C₁₄H₁₉N₄O₄⁺ [M+H]⁺ 307.14, found 307.21.

### Methyl (S)-5-(4-(4-amino-2-cyanophenyl)-1H-1,2,3-triazol-1-yl)-2-(((benzyloxy)carbonyl) amino)-pentanoate (XX44)

CuSO₄·5H₂O (1.05 g, 4.22 mmol) in water (52 mL) was added to a solution of azide XX43 (1.70 g, 5.55 mmol) and alkyne XX40 (0.789 g, 5.55 mmol) in THF (280 mL) at RT. The solution was purged with N₂ for 20 min, then sodium ascorbate (1.649 g, 8.32 mmol) in water (52 mL) was added at RT. After stirring for 30 min, DMF (40.0 mL) was added and stirring was continued for 88 h at RT. A water/brine (1:1, 220 mL) mixture was added and the product was then extracted with EtOAc/heptane (1:1, 2×200 mL). The combined organic layers were washed with brine (2×100 mL), dried over MgSO₄, filtered and concentrated. Purification by flash chromatography (silica gel, heptane:EtOAc 1:0 to 0:1) afforded triazole XX44 (2.09 g, 84%) as a yellow sticky oil.

¹H NMR (400 MHz, CDCl₃) ppm=8.18 (s, 1H), 8.10 (d, J=8.4 Hz, 1H), 7.38-7.28 (m, 5H), 6.98-6.93 (m, 2H), 5.40 (br d, J=7.8 Hz, 1H), 5.11 (s, 2H), 4.47-4.40 (m, 3H), 3.98 (s, 2H), 3.74 (s, 3H), 2.10-1.89 (m, 3H), 1.76-1.66 (m, 1H).

MS (ESI⁺) calc. for C₂₃H₂₅N₆O₄⁺ [M+H]449.19, found 449.31.

### Methyl (S)-2-amino-5-(4-(4-amino-2-cyanophenyl)-1H-1,2,3-triazol-1-yl)pentanoate (XX45)

Triazole XX44 (1.12 g, 2.497 mmol) was reacted with Pd/C (10% palladium on activated carbon, 0.133 g, 0.125 mmol) in EtOAc (22 mL) under H₂-atmosphere at RT. After 16 h, the flask was purged with N₂ and more Pd/C (10% palladium on activated carbon, 0.200 g, 0.188 mmol) was added at RT. Hydrogen gas was reintroduced and the mixture was stirred for a further 4.5 h at RT. The flask was purged with N₂ and filtered over Celite. HCl in dioxane (4.0 N, 2.0 mL) was then added to the filtrate. The volume of the filtrate was reduced to approximately 50 mL by rotary evaporation, and the suspension was then filtered, and the solid washed with EtOAc and ether. The white solid was dried under vacuum to give amine XX45 (849 mg, 88%) as the dihydrochloride salt.

¹H NMR (400 MHz, methanol-d₄) ppm=8.66 (s, 1H), 8.21 (d, J=8.5 Hz, 1H), 7.78 (d, J=2.4 Hz, 1H), 7.71 (dd, J=8.5, 2.4 Hz, 1H), 4.63 (t, J=6.6 Hz, 2H), 4.16 (t, J=6.4 Hz, 1H), 3.87 (s, 3H), 2.27-1.91 (m, 4H).

MS (ESI⁺) calc. for C₁₅H₁₉N₆O₂⁺ [M+H]⁺ 315.16, found 315.25.

### Methyl (S)-5-(4-(4-amino-2-cyanophenyl)-1H-1,2,3-triazol-1-yl)-2-(4-(N-((2,4-diaminopteridin-6-yl)methyl)formamido)benzamido)pentanoate (XX46)

The dihydrochloride salt of amine XX45 (498 mg, 1.29 mmol) in DMF (10 mL) was reacted with amine XT48 (397 mg, 1.17 mmol) according to general procedure XXA. After concentration, the crude product was stirred in MeCN (10 mL) for 3 days at RT and was then filtered. The solid was washed with MeCN and ether, and finally dried under vacuum to give amide XX46 (688 mg, 93%) as an orange solid.

MS (ESI⁺) calc. for C₃₀H₃₀N₁₃O₄⁺ [M+H]⁺ 636.25, found 636.45.

### (S)-5-(4-(4-Amino-2-cyanophenyl)-1H-1,2,3-triazol-1-yl)-2-(4-(((2,4-diaminopteridin-6-yl)methyl)amino)benzamido)pentanoic acid (XX47)

The hydrolysis of ester XX46 (330 mg, 0.519 mmol) was carried out according to general procedure XXC. A portion of the crude product was purified by RP-HPLC (water×0.10% TFA/MeCN×0.1% TFA, gradient 10% to 30%) to give acid XX47 as a yellow solid.

¹H NMR (400 MHz, DMSO-d₆) ppm=12.42 (br s, 1H), 9.22 (s, 1H), 9.18 (s, 1H), 8.77 (s, 1H), 8.51 (br s, 1H), 8.32 (s, 1H), 8.15 (d, J=7.8 Hz, 1H), 7.81 (br s, 1H), 7.66 (d, J=8.8 Hz, 2H), 7.60 (d, J=8.9 Hz, 1H), 6.87 (s, 1H), 6.88 (dd, J=9.0, 2.4 Hz, 1H), 6.84 (br s, 1H), 6.67 (d, J=8.8 Hz, 2H), 4.55 (s, 2H), 4.39 (t, J=6.9 Hz, 2H), 4.36-4.30 (m, 1H), 1.96-1.81 (m, 2H), 1.79-1.65 (m, 2H).

MS (ESI⁺) calc. for C₂₈H₂₈N₁₃O₃⁺ [M+H]⁺ 594.24, found 594.39.

### Preparation of (S)-2-(4-(((2,4-diaminopteridin-6-yl)methyl)amino)benzamido)-5-(4-((S)-2-((S)-2-(6-(2,5-dioxo-2,5-dihydro-1H-pyrrol-1-yl)hexanamido)-3-methylbutanamido) propanamido)-2-hydroxybenzamido)pentanoic acid (XX28)

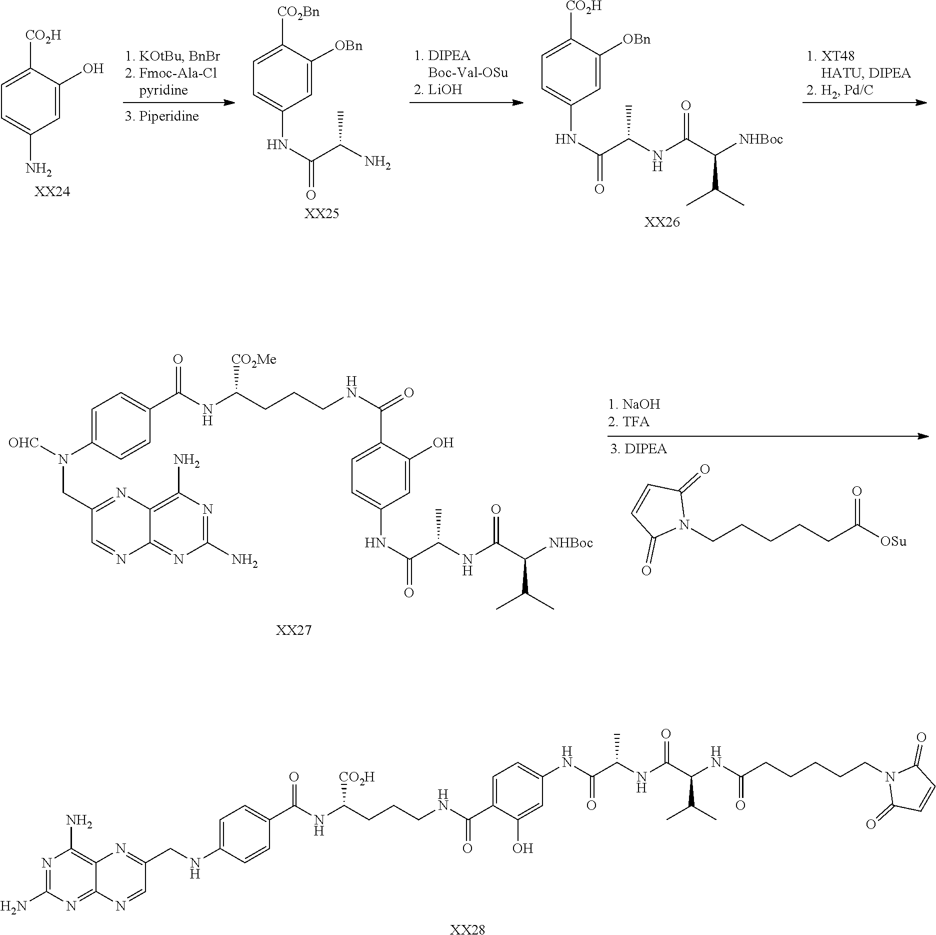

### Benzyl (S)-4-(2-aminopropanamido)-2-(benzyloxy)benzoate (XX25)

To a stirred solution of 4-amino-2-hydroxybenzoic acid (XX24; 5.00 g, 32.7 mmol) in DMF (150 mL) at 0° C., was added KOtBu (4.03 g, 35.9 mmol). After 15 min, BnBr (4.27 mL, 35.9 mmol) was added dropwise, and the suspension was allowed to stir at RT for a further 4 h before the reaction vessel was again cooled to 0° C. More KOtBu (4.03 g, 35.9 mmol) and BnBr (4.27 mL, 35.9 mmol) was added at this point. The reaction was stirred overnight and was subsequently quenched with water and extracted with EtOAc (3×). The combined organic layers were diluted 2:1 with heptane, and were then washed with water (2×) and brine, dried over Na₂SO₄ and concentrated on silica gel. Purification by flash chromatography (silica gel, 0-60% EtOAc in heptane) afforded the benzyl ester (5.30 g).

The product was reacted with Fmoc-Ala-Cl according to the procedure for XT11. After concentration of the reaction mixture, the crude was dissolved in EtOAc and washed with HCl (0.1 M) until the water layer remained acidic. The combined water layers were backextracted with EtOAc, and the combined organic layers were washed with sat. aq. NaHCO₃ and brine, dried over MgSO₄, filtered and concentrated to give a sticky pale yellow gum (11.08 g).

MS (ESI⁺) calc. for C₃₉H₃₄N₂NaO₆ [M+Na]+649.23, found 649.44.

The crude was dissolved in DMF (122 mL) and piperidine (49 mL) was added at RT. After 3 min, the reaction was concentrated and the crude was purified by flash chromatography (silica gel, 0-20% MeOH in DCM) to afford amine XX25 (4.45 g, 34%, 3 steps).

¹H NMR (400 MHz, CDCl₃) ppm=9.73 (br s, 1H), 7.89 (d, J=8.5 Hz, 1H), 7.86 (d, J=1.6 Hz, 1H), 7.48 (d, J=7.5 Hz, 2H), 7.41-7.37 (m, 2H), 7.36-7.28 (m, 6H), 6.90 (dd, J=8.5, 1.8 Hz, 1H), 5.33 (s, 2H), 5.19 (s, 2H), 3.63 (q, J=7.0 Hz, 1H), 1.65 (br s, 2H), 1.44 (d, J=7.0 Hz, 3H).

MS (ESI⁺) calc. for C₂₄H₂₅N₂O₄ [M+H]⁺ 405.18, found 405.29.

### 2-(Benzyloxy)-4-((S)-2-((S)-2-((tert-butoxycarbonyl)amino)-3-methylbutanamido) propanamido)benzoic acid (XX26)

DIPEA (4.81 mL, 27.5 mmol) and Boc-Val-OSu (3.81 g, 12.1 mmol) were added to a solution of amine XX25 (4.45 g, 11.0 mmol) in DMF (50 mL) at RT. The mixture was stirred for 5 h and was then poured into water. The mixture was extracted with EtOAc/heptane (1:1, 3×) The combined organic layers were washed with water (2×) and brine, dried over MgSO₄, filtered and concentrated. Purification by flash chromatography (0-100% EtOAc in heptane)) afforded the dipeptide (6.57 g).

The crude product was taken up in THF (39 mL)/MeOH (8 mL) and lithium hydroxide hydrate (1.69 g, 40.3 mmol) in water (15.7 mL) was added at RT. The mixture was stirred for 19 h after which the organics were then removed by rotary evaporation. Water (300 mL) was added and the mixture was filtered, washed with water (100 mL) and the solid was washed with ether (2×150 mL). Both ether fractions were separately used to wash the water layer. EtOAc (250 mL) was added to the water layer and HCl (1.0 M, 41 mL) was added followed by the solids. After mixing, the product dissolved in the organic phase. The water layer was washed with EtOAc (2×100 mL) and the combined organic layers, including the ether fractions, were washed with brine, dried over Na₂SO₄, filtered and concentrated. A white solid formed that was then suspended in ˜1:1 EtOAc/heptane (˜20 mL) and filtered off to give acid XX26 (3.78 g, 67%, 2 steps) as a white solid.

¹H NMR (400 MHz, DMSO-d₆) ppm=12.35 (br s, 1H), 10.23 (s, 1H), 8.12 (d, J=6.7 Hz, 1H), 7.70 (d, J=8.5 Hz, 1H), 7.55 (d, J=1.2 Hz, 1H), 7.52 (d, J=7.5 Hz, 2H), 7.39 (t, J=7.5 Hz, 2H), 7.34-7.30 (m, 1H), 7.23 (dd, J=8.5, 1.4 Hz, 1H), 6.71 (d, J=8.7 Hz, 1H), 5.14 (s, 2H), 4.42 (quint, J=7.0 Hz, 1H), 3.85 (t, J=7.8 Hz, 1H), 2.00-1.90 (m, 1H), 1.39 (s, 9H), 1.31 (d, J=7.1 Hz, 3H), 0.88 (d, J=6.7 Hz, 3H), 0.82 (d, J=6.7 Hz, 3H).

MS (ESI⁺) calc. for C₂₇H₃₆N₃O₇ [M+H]⁺ 514.26, found 514.61.

### Methyl (S)-5-(4-((S)-2-((S)-2-((tert-butoxycarbonyl)amino)-3-methylbutanamido) propanamido)-2-hydroxybenzamido)-2-(4-(N-((2,4-diaminopteridin-6-yl)methyl)formamido) benzamido)pentanoate (XX27)

Acid XX26 (421 mg, 0.820 mmol) was reacted with HATU (374 mg, 0.983 mmol) and XT48 (413 mg, 0.820 mmol) according to general procedure XXA. Purification by flash chromatography (silica gel, DCM:MeOH 1:0 to 4:1) afforded a yellow solid that was suspended in hot MeCN (6 mL). After cooling to RT, the solid was filtered off and stirred in water for 10 min. After filtration and drying, the crude benzyl ether (200 mg, 0.208 mmol) was reacted with Pd(OH)₂/C (20% on activated carbon, 100 mg) in AcOH (2.0 mL) under H₂ atmosphere at RT for 2 h and 20 min. The reaction was then purged with N₂ and filtered over Celite. After concentration, the material was coevaporated with toluene. The solid was dissolved in DMF (1 mL)/DCM (3 mL) and MnO₂ (300 mg) was added at RT. After stirring for 3 h, the reaction was filtered and concentrated. Purification by flash chromatography (silica gel, 0-20% MeOH in DCM) afforded phenol XX27 (79 mg, 11%, 2 steps).

MS (ESI⁺) calc. for C₄₁H₅₃N₁₂O₁₀ [M+H]⁺ 873.40, found 873.90.

### (S)-2-(4-(((2,4-Diaminopteridin-6-yl)methyl)amino)benzamido)-5-(4-((S)-2-((S)-2-(6-(2,5-dioxo-2,5-dihydro-1H-pyrrol-1-yl)hexanamido)-3-methylbutanamido)propanamido)-2-hydroxybenzamido)pentanoic acid (XX28)

Phenol XX27 (79 mg, 0.090 mmol) was reacted with NaOH as described in general procedure XXC, to afford the acid (75 mg) as a green/grey solid. The material was suspended in DCM (2.0 mL) and TFA (2.0 mL) was added at 0° C. After 30 min, the reaction was concentrated. The brown oil was dissolved in DMF (3 mL), and 6-maleimidohexanoic acid N-hydroxylsuccinimide ester (29.1 mg, 0.094 mmol) and DIPEA (0.063 mL, 0.360 mmol) were added at RT. More DIPEA (0.031 mL, 0.180 mmol) and 6-maleimidohexanoic acid N-hydroxylsuccinimide ester (5.55 mg, 0.018 mmol) was added after 20 min and 90 min, respectively, and the reaction was stopped after 2 h. The mixture was concentrated and the residue was purified by preparative RP-HPLC (water×0.1% TFA/MeCN×0.1% TFA, gradient 20% to 50%). Organic solvent was removed by rotary evaporation and the aq. solution was then lyophilized. The solid was once more purified by preparative RP-HPLC using a different eluent (water×0.1% TFA/MeOH:MeCN (1:1)×0.1% TFA, gradient 20% to 50%). Organic solvent was removed by rotary evaporation and the aq. solution was then lyophilized to give phenol XX28 (3.1 mg) as a yellow solid.

MS (ESI⁺) calc. for C₄₄H₅₄N₁₃O₁₀ [M+H]⁺ 924.41, found 924.91.

### Preparation of (S)-5-(4-aminobenzamido)-2-(4-(2-(2,4-diaminoquinazolin-6-yl)ethyl) benzamido)pentanoic acid (XJ4)

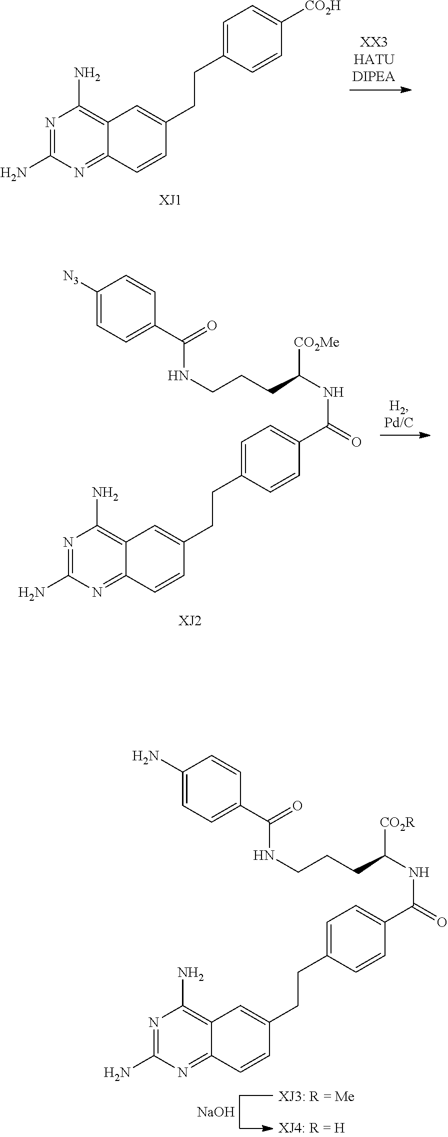

### Methyl (S)-5-(4-azidobenzamido)-2-(4-(2-(2,4-diaminoquinazolin-6-yl)ethyl)benzamido) pentanoate (XJ2)

Acid XJ1 (514 mg, 1.67 mmol, synthesized as described in US 2005/0020833 and US 2009/0253719) was reacted with amine XX3 (534 mg, 1.83 mmol) according to general procedure XXA. Purification by flash chromatography (silica gel, MeOH:DCM 0:1 to 1:4) afforded ester XJ2 (474 mg, 49%) as a yellow solid.

MS (ESI⁺) calc. for C₃₀H₃₂N₉O₄⁺ [M+H]⁺ 582.25, found 582.62.

### Methyl (S)-5-(4-aminobenzamido)-2-(4-(2-(2,4-diaminoquinazolin-6-yl)ethyl)benzamido) pentanoate (XJ3)

Reduction of azide XJ2 (474 mg, 0.815 mmol) was carried out according to general procedure XXB to give aniline XJ3 (453 mg, quant) as a yellow solid.

MS (ESI⁺) CALC. FOR C₃₀H₃₄N₇O₄⁺ [M+H]⁺ 556.26, FOUND 556.62.

### (S)-5-(4-Aminobenzamido)-2-(4-(2-(2,4-diaminoquinazolin-6-yl)ethyl)benzamido)pentanoic acid (XJ4)

The hydrolysis of ester XJ3 (45 mg, 0.081 mmol) was carried out according to general procedure XXC, to give acid XJ4 (9 mg, 20%) as a yellow solid.

¹H NMR (400 MHz, DMSO-d₆) ppm=8.40 (br d, J=7.5 Hz, 1H), 8.00 (br t, J=5.4 Hz, 1H), 7.91 (s, 1H), 7.80 (d, J=8.1 Hz, 2H), 7.55 (d, J=8.5 Hz, 2H), 7.55 (br s, 2H), 7.43 (br d, J=8.7 Hz, 1H), 7.31 (d, J=8.0 Hz, 2H), 7.16 (d, J=8.5 Hz, 1H), 6.52 (d, J=8.5 Hz, 2H), 6.51 (br s, 2H), 5.56 (br s, 2H), 4.35-4.29 (m, 1H), 3.24-3.19 (m, 2H), 3.00-2.93 (m, 4H), 1.88-1.71 (m, 2H), 1.62-1.53 (m, 2H).

MS (ESI⁺) calc. for C₂₉H₃₂N₇O₄⁺ [M+H]⁺ 542.24, found 542.62.

### Preparation of (S)-2-(4-(2-(2,4-diaminoquinazolin-6-yl)ethyl)benzamido)-5-(4-((S)-2-((S)-2-(6-(2,5-dioxo-2,5-dihydro-1H-pyrrol-1-yl)hexanamido)-3-methylbutanamido) propanamido) pentanoic acid (XJ9)

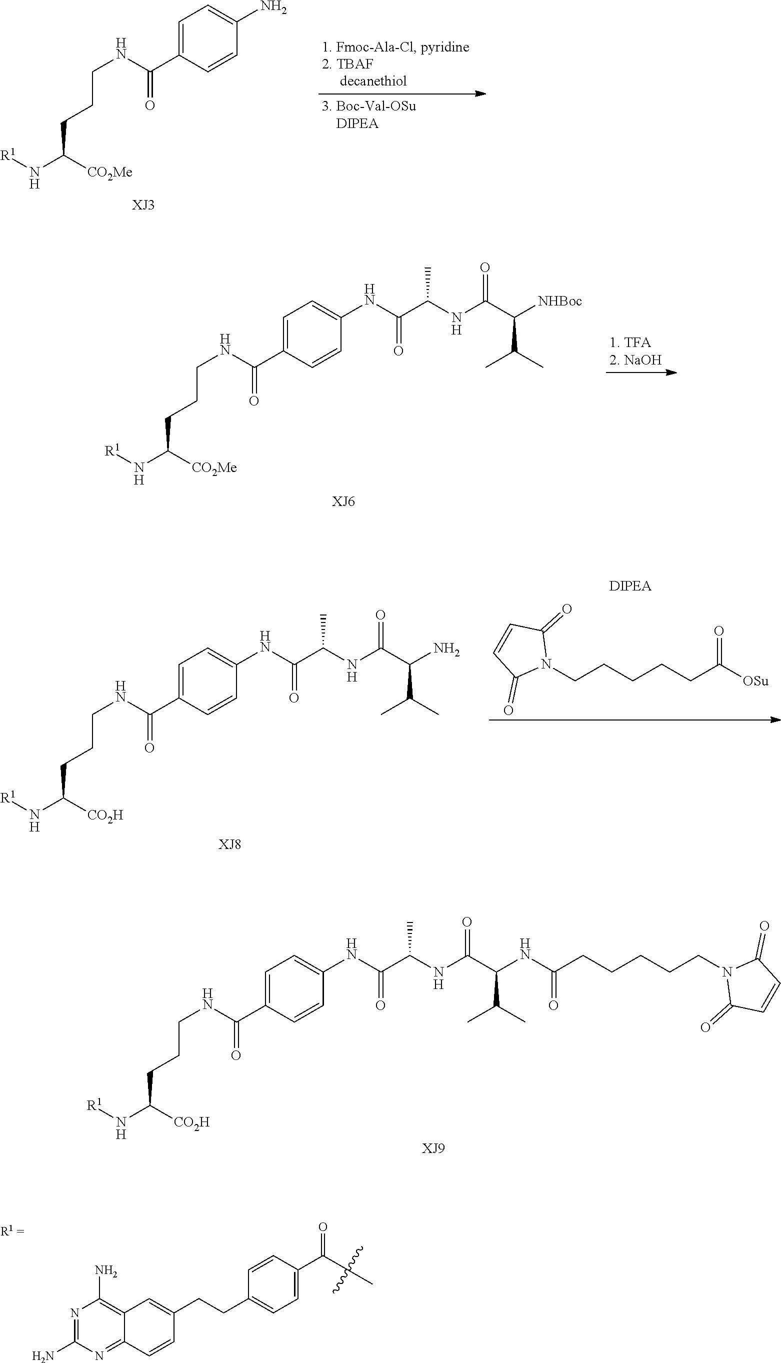

### Methyl (S)-5-(4-((S)-2-((S)-2-((tert-butoxycarbonyl)amino)-3-methylbutanamido) propanamido)benzamido)-2-(4-(2-(2,4-diaminoquinazolin-6-yl)ethyl)benzamido)pentanoate (XJ6)

Aniline XJ3 (405 mg, 0.729 mmol) was reacted with Fmoc-Ala-Cl (481 mg, 1.45 mmol) according to the procedure for XT11, affording amide XJ5 (quant) as a yellow solid.

MS (ESI⁺) calc. for C₄₈H₄₉N₈O₇⁺ [M+H]⁺ 849.36, found 849.49.

Fmoc-protected amine XJ5 (619 mg, 0.729 mmol) was deprotected with TBAF·3H₂O and decanethiol, and subsequently reacted with Boc-Val-OSu, analogous to the conversion of XT11 to XT12. Purification of the crude by flash chromatography (silica gel, MeOH:DCM 0:1 to 1:4) afforded dipeptide XJ6 (317 mg, 52%), contaminated with tetrabutylamine salts.

MS (ESI⁺) calc. for C₄₃H₅₆N₉O₈⁺ [M+H]⁺ 826.42, found 826.83.

### (S)-5-(4-((S)-2-((S)-2-Amino-3-methylbutanamido)propanamido)benzamido)-2-(4-(2-(2,4-diaminoquinazolin-6-yl)ethyl)benzamido)pentanoic acid (XJ8)

Dipeptide XJ6 (310 mg, 0.375 mmol) was deprotected according to the procedure for XT13. The crude product (272 mg) was hydrolyzed with NaOH according to general procedure XXC, to give XJ8 (84 mg, 32%) as an off-white solid.

MS (ESI⁺) calc. for C₃₇H₄₆N₉O₆⁺ [M+H]⁺ 712.35, found 712.80.

### (S)-2-(4-(2-(2,4-Diaminoquinazolin-6-yl)ethyl)benzamido)-5-(4-((S)-2-((S)-2-(6-(2,5-dioxo-2,5-dihydro-1H-pyrrol-1-yl)hexanamido)-3-methylbutanamido)propanamido) pentanoic acid (XJ9)

Amine XJ8 (76 mg, 0.107 mmol) was reacted with 6-maleimidohexanoic acid N-hydroxylsuccinimide ester (32.9 mg, 0.107 mmol) and DIPEA (0.112 mL, 0.64 mmol) according to the procedure for XT17. The crude solid was purified by preparative RP-HPLC (water×0.1% TFA/MeCN×0.1% TFA, gradient 5% to 95%). Product fractions were pooled, MeCN was removed by rotary evaporation, and the aq. solution was lyophilized to yield XJ9 (21.6 mg, 22%) as a white solid.

¹H NMR (400 MHz, DMSO-d₆) ppm=12.36 (br s, 1H), 10.10 (br s, 1H), 8.88 (br s, 1H), 8.75 (br s, 1H), 8.54 (d, J=7.7 Hz, 1H), 8.35 (t, J=5.6 Hz, 1H), 8.18 (d, J=6.8 Hz, 1H), 8.13 (d, J=1.0 Hz, 1H), 7.82-7.78 (m, 7H), 7.68-7.64 (m, 3H), 7.35 (d, J=8.4 Hz, 1H), 7.31 (br d, J=8.3 Hz, 2H), 6.99 (s, 2H), 4.42-4.35 (m, 2H), 4.17 (dd, J=8.5, 7.0 Hz, 1H), 3.29-3.25 (m, 4H), 3.01 (br s, 4H), 2.22-2.07 (m, 2H), 2.00-1.89 (m, 1H), 1.89-1.74 (m, 2H), 1.69-1.58 (m, 2H), 1.51-1.44 (m, 4H), 1.31 (d, J=7.1 Hz, 3H), 1.22-1.14 (m, 2H), 0.87 (d, J=6.6 Hz, 3H), 0.83 (d, J=6.7 Hz, 3H).

MS (ESI⁺) calc. for C₄₇H₅₇N₁₀O₉⁺ [M+H]⁺ 905.42, found 905.94.

### Preparation of (S)-5-amino)-2-(4-(((2,4-diaminopteridin-6-yl)methyl)amino) benzamido)pentanoic acid (XJ11) and (S)-2-(4-(((2,4-diaminopteridin-6-yl)methyl)amino) benzamido)-5-((((4-((2S,5S)-13-(2,5-dioxo-2,5-dihydro-1H-pyrrol-1-yl)-5-isopropyl-4,7-dioxo-2-(3-ureidopropyl)-8,11-dioxa-3,6-diazatridecanamido)benzyl)oxy)carbonyl)amino) pentanoic acid (XJ13)

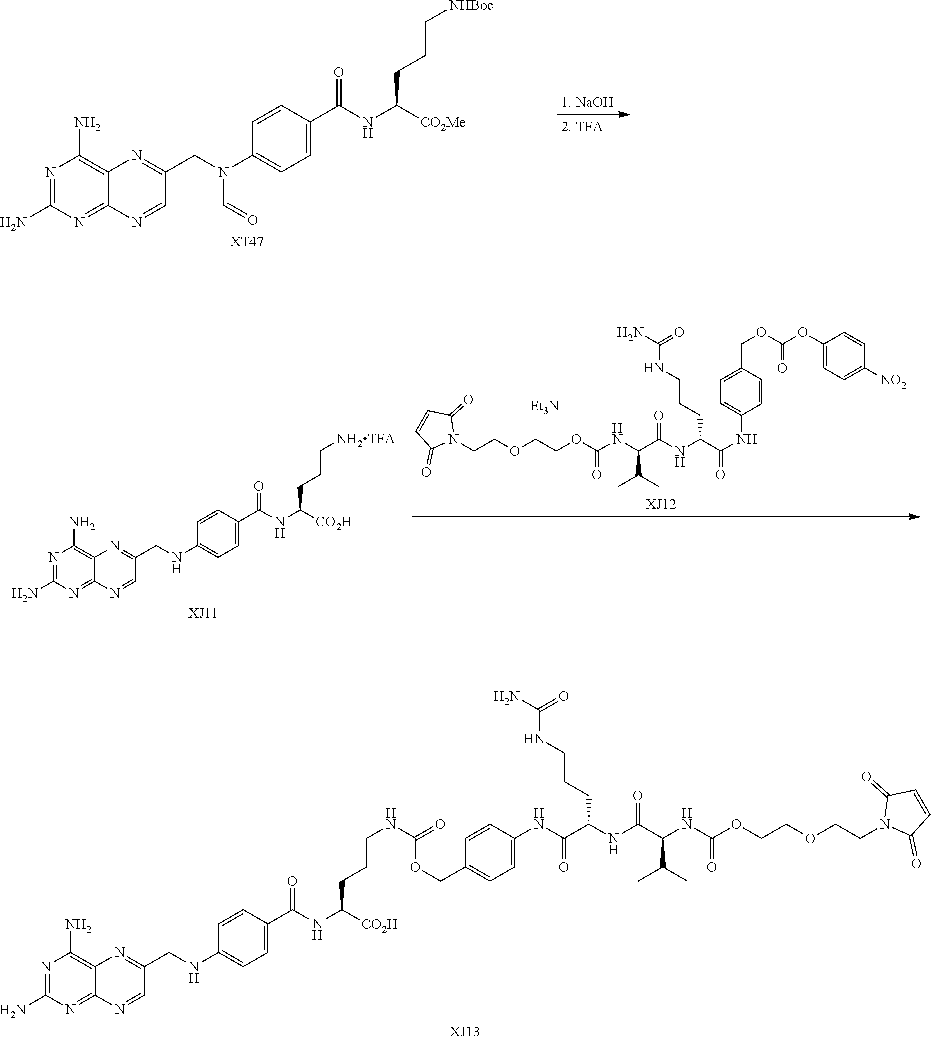

### (S)-5-Amino)-2-(4-(((2,4-diaminopteridin-6-yl)methyl)amino)benzamido)pentanoic acid (XJ11)

The hydrolysis of formamide XT47 (270 mg, 0.476 mmol) with NaOH (12 eq) was carried out according to general procedure XXC. The product (178 mg) was then deprotected with TFA according to the procedure for XT13. A portion of the crude product (40 mg) was purified by preparative RP-HPLC (water×0.1% TFA/MeCN×0.1% TFA, gradient 5% to 35%). Product fractions were pooled, MeCN was removed by rotary evaporation, and the aq. solution was lyophilized to yield XJ11 (6.7 mg) as a yellow solid.

¹H NMR (400 MHz, DMSO-d₆) ppm=12.6 (br s, 1H), 9.19 (br s, 1H), 8.86 (s, 1H), 8.45 (br s, 1H), 8.25 (d, J=8.0 Hz, 1H), 7.90 (br s, 1H), 7.78-7.73 (m, 4H), 6.95 (br s, 1H), 6.79 (d, J=8.7 Hz, 2H), 4.65 (s, 2H), 4.44-4.38 (m, 1H), 2.88-2.79 (m, 2H), 1.95-1.88 (m, 2H), 1.84-1.76 (m, 2H), 1.71-1.61 (m, 2H).

¹³C NMR (100 MHz, DMSO-d₆) ppm=174.2, 166.7, 163.3, 151.0, 129.5, 122.0, 121.9, 111.8, 62.4, 52.2, 46.0, 28.1, 25.9, 24.6.

MS (ESI⁺) calc. for C₁₉H₂₄N₉O₃⁺ [M+H]⁺ 426.19, found 426.31.

### (S)-2-(4-(((2,4-Diaminopteridin-6-yl)methyl)amino)benzamido)-5-((((4-((2S,5S)-13-(2,5-dioxo-2,5-dihydro-1H-pyrrol-1-yl)-5-isopropyl-4,7-dioxo-2-(3-ureidopropyl)-8,11-dioxa-3,6-diazatridecanamido)benzyl)oxy)carbonyl)amino)pentanoic acid (XJ13)

Amine XJ11 (234 mg, 0.358 mmol) was reacted with XJ12 (271 mg, 0.358 mmol) (see: Elgersma et al, Mol. Pharm. 2015, 12, 1813-35) in DMF (4 mL) at 0° C. in the presence of Et₃N (0.150 mL, 1.07 mmol) for 1.5 h, during which time the temperature was allowed to gradually reach RT. After concentration, the crude solid was purified by preparative RP-HPLC (water×0.1% TFA/MeCN×0.1% TFA, gradient 25% to 75%). Product fractions were pooled, MeCN was removed by rotary evaporation, and the aq.solution was lyophilized to yield XJ13 (99 mg, 27%) as a yellow solid.

¹H NMR (400 MHz, DMSO-d₆) ppm=13.1, (br s, 1H), 12.49 (br s, 1H), 10.02 (s, 1H), 9.35 (s, 1H), 9.31 (s, 1H), 8.84 (s, 1H), 8.63 (br s, 1H), 8.15 (d, J=7.7 Hz, 1H), 8.07 (d, J=7.3 Hz, 1H), 7.73 (d, J=8.7 Hz, 2H), 7.57 (d, J=8.5 Hz, 2H), 7.27 (d, J=8.5 Hz, 2H), 7.21-7.17 (m, 2H), 7.02 (s, 2H), 6.75 (m, 3H), 6.00 (br s, 2H), 4.93 (s, 2H), 4.62 (s, 2H), 4.43-4.39 (m, 1H), 4.34-4.28 (m, 1H), 4.06-3.97 (m, 2H), 3.90-3.87 (m, 1H), 3.57-2.52 (m, 6H), 3.03-2.93 (m, 4H), 2.00-1.91 (m, 1H), 1.83-1.78 (m, 1H), 1.73-1.66 (m, 1H), 1.63-1.34 (m, 5H), 0.86 (d, J=6.7 Hz, 3H), 0.82 (d, J=6.7 Hz, 3H).

MS (ESI⁺) calc. for C₄₇H₆₀N₁₅O₁₃⁺ [M+H]⁺ 1042.44, found 1042.92.

### Preparation of (S)-2-(5-(2,5-dioxo-2,5-dihydro-1H-pyrrol-1-yl)pentanamido)-N1,N5-di(prop-2-yn-1-yl)pentanediamide (XS2)

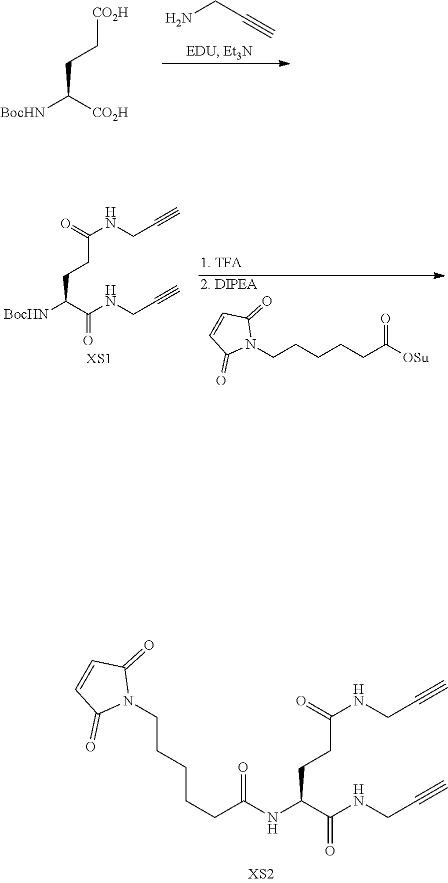

### tert-Butyl (S)-(1,5-dioxo-1,5-bis(prop-2-yn-1-ylamino)pentan-2-yl)carbamate (XS1)

To a suspension of Boc-Glu-OH (769 mg, 3.11 mmol) and propargylamine (438 μL, 6.85 mmol) in DCM (15 mL) were added Et₃N (1.74 mL, 12.5 mmol) and 1-ethyl-3-(3-dimethylaminopropyl)carbodiimide (EDC) (1.49 g, 7.78 mmol) at 0° C. The reaction mixture was allowed to reach RT and was stirred overnight. The reaction mixture was concentrated, taken up in EtOAc (30 mL) and washed with water (30 mL). The water layer was extracted with EtOAc (2×30 mL) and the combined organic layers were washed with sat. aq. NaHCO₃ (2×30 mL) and brine (30 mL), dried over Na₂SO₄ and concentrated. Water layers still containing product according to UPLC-MS analysis, were extracted with DCM (2×20 mL) and the combined DCM layers were washed with brine (30 mL), dried over Na₂SO₄, combined with the crude product of the EtOAc extraction step, and evaporated in vacuo. The crude was purified by flash chromatography (silica gel, MeOH:DCM 0:1 to 1:4) to give dialkyne XS1 (440 mg, 44%) as a pale yellow solid.

¹H NMR (400 MHz, CDCl₃) ppm=7.05 (br s, 1H), 6.40 (br s, 1H), 5.67 (d, J=7.4 Hz, 1H), 4.25-4.13 (m, 1H), 4.12-3.99 (m, 4H), 2.45-2.29 (m, 2H), 2.24 (dt, J=7.5, 2.6 Hz, 2H), 2.16-2.05 (m, 1H), 2.05-1.94 (m, 1H), 1.44 (s, 9H).

¹³C NMR (100 MHz, CDCl₃) ppm=172.4, 171.4, 79.4, 79.3, 71.7, 53.5, 32.4, 29.3, 29.1, 28.3.

MS (ESI⁺) calc. for C₁₆H₂₄N₃O₄⁺ [M+H]⁺ 322.18, found 322.44.

### (S)-2-(5-(2,5-Dioxo-2,5-dihydro-1H-pyrrol-1-yl)pentanamido)-N1,N5-di(prop-2-yn-1-yl) pentanediamide (XS2)

Dialkyne XS1 (430 mg, 1.34 mmol) was dissolved in DCM (5.0 mL) and cooled to 0° C. TFA (5.0 mL) was added and the reaction mixture was allowed to reach RT and stirred for 1 h. The reaction mixture was coevaporated with toluene (5.0 mL) and toluene:DCM (6 mL, 5:1) and dried on high vacuum, to give the amine as a red brown oil. The material was dissolved in DMF (10 mL), and 6-maleimidohexanoic acid N-hydroxylsuccinimide ester (495 mg, 1.61 mmol) and DIPEA (1.40 mL, 8.03 mmol) were then added at RT. After 18 h, the reaction mixture was concentrated, and coevaporated with DCM:toluene (15 mL, 2:1). The crude was purified by flash chromatography (silica gel, MeOH:DCM 0:1 to 1:4) to give maleimide XS2 (307 mg, 55%, 2 steps) as a white solid.

¹H NMR (400 MHz, DMSO-d₆) ppm=8.30 (t, J=5.5 Hz, 1H), 8.24 (t, J=5.8 Hz, 1H), 7.96-7.88 (m, 1H), 7.00 (s, 2H), 4.23-4.14 (m, 1H), 3.83 (td, J=2.8, 2.4 Hz, 4H), 3.41-3.35 (m, 2H), 3.08 (dt, J=7.1, 2.5 Hz, 2H), 2.16-2.01 (m, 4H), 1.90-1.79 (m, 1H), 1.74-1.62 (m, 1H), 1.48 (quint, J=7.4 Hz, 4H), 1.27-1.13 (m, 2H).

¹³C NMR (100 MHz, DMSO-d₆) ppm=172.6, 171.7, 171.6, 171.6, 134.9, 81.7, 81.5, 73.4, 73.3, 52.4, 37.5, 35.4, 32.0, 28.4, 28.3, 28.2, 26.3, 25.7, 25.1.

MS (ESI⁺) calc. for C₂₁H₂₇N₄O₅⁺ [M+H]⁺ 415.20, found 415.34.

### Preparation of (S)-5-(2-chloro-4-((S)-2-((S)-2-(6-(2,5-dioxo-2,5-dihydro-1H-pyrrol-1-yl) hexanamido)-3-methylbutanamido)propanamido)benzamido)-2-(4-(((2,4-diaminopteridin-6-yl)methyl)amino)benzamido)pentanoic acid (XS6)

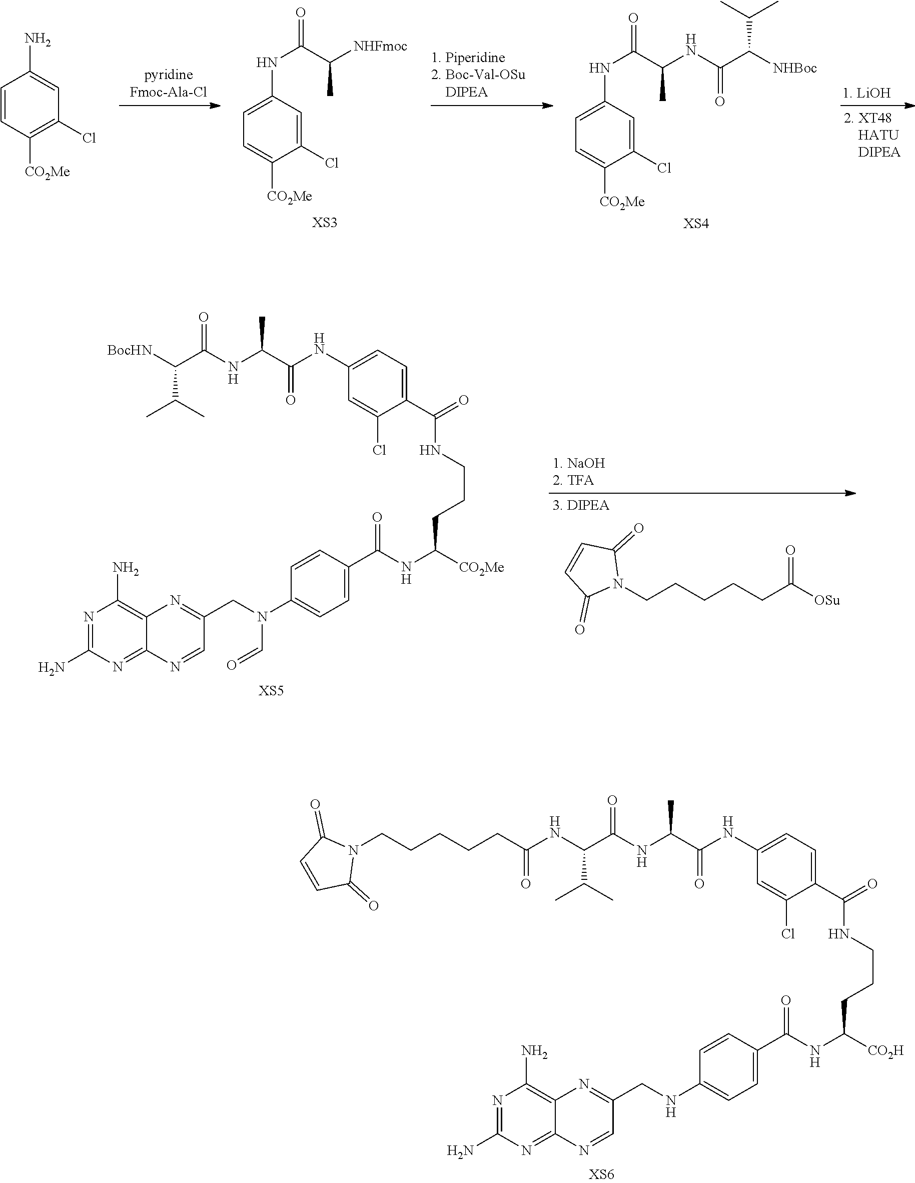

### Methyl (S)-4-(2-((((9H-fluoren-9-yl)methoxy)carbonyl)amino)propanamido)-2-chlorobenzoate (XS3)

Methyl 4-amino-2-chlorobenzoate (1.00 g, 5.39 mmol) was reacted with Fmoc-Ala-Cl according to the procedure for XT11. After concentration of the quenched reaction mixture and coevaporation with toluene (20 mL), the mixture was partly dissolved in EtOAc (40 mL) and extracted with 0.1 M HCl (2×15 mL). The water layer was extracted with EtOAc (20 mL) and the combined organic layers were washed with aq. NaHCO₃ (2×15 mL) and brine (2×15 mL), dried over MgSO₄, filtered and concentrated, to yield amide XS3 (2.63 g, quant, 2 steps) as a light yellow solid.

MS (ESI⁺) calc. for C₂₆H₂₄ClN₂O₅⁺ [M+H]⁺ 479.14, found 479.30.

### Methyl 4-((S)-2-((S)-2-((tert-butoxycarbonyl)amino)-3-methylbutanamido) propanamido)-2-chlorobenzoate (XS4)

To carbamate XS3 (2.63 g, 5.49 mmol) was added piperidine (39.0 mL, 395 mmol) and the resulting solution was stirred for 15 min. The reaction mixture was concentrated and purified by flash chromatography (silica gel, MeOH:DCM 0:1 to 1:3) to yield the deprotected product (1.17 g, 83%) as a light brown oil. A portion of the material (1.15 g, 4.48 mmol) was dissolved in DMF (20 mL). DIPEA (1.96 mL, 11.2 mmol) and Boc-Val-OSu (1.55 g, 4.93 mmol) were then added at RT and the resulting solution was stirred at RT for 3 h. The reaction mixture was poured into water (240 mL) and the water layer was extracted with EtOAc:heptane (1:1, 3×80 mL). The combined organic layers were washed with water (100 mL) and brine (2×50 mL), dried over MgSO₄, filtered and concentrated. The crude was purified by flash chromatography (silica gel, EtOAc:heptane 0:1 to 1:1) to yield amide XS4 (1.51 g, 74%) as an off-white solid.

¹H NMR (400 MHz, CDCl₃) ppm=9.16 (br s, 1H), 7.86-7.80 (m, 2H), 7.73-7.55 (m, 1H), 6.65 (d, J=6.3 Hz, 1H), 4.98 (d, J=6.1 Hz, 1H), 4.67 (quint, J=7.2 Hz, 1H), 3.96 (t, J=5.5 Hz, 1H), 3.90 (s, 3H), 2.27-2.17 (m, 1H), 1.49-1.46 (m, 3H), 1.44 (s, 9H), 1.01 (d, J=6.8 Hz, 3H), 0.96 (d, J=6.9 Hz, 3H).

¹³C NMR (100 MHz, CDCl₃) ppm=172.1, 170.5, 165.5, 156.5, 142.0, 134.9, 132.6, 124.4, 121.6, 117.2, 81.2, 60.8, 52.2, 49.8, 30.2, 28.3, 24.8, 19.3, 17.7, 17.3.

MS (ESI⁺) calc. for C₂₁H₃₁ClN₃O₆⁺ [M+H]⁺ 456.19, found 456.56.

### Methyl (S)-5-(4-((S)-2-((S)-2-((tert-butoxycarbonyl)amino)-3-methylbutanamido) propanamido)-2-chlorobenzamido)-2-(4-(N-((2,4-diaminopteridin-6-yl)methyl)formamido) benzamido)pentanoate (XS5)

Ester XS4 (0.750 g, 1.65 mmol) was suspended in dioxane (17 mL) and water (10 mL). Aq. LiOH (2.0 M, 3.29 mL, 6.58 mmol) was added and the reaction mixture was stirred at RT for 4 h. More dioxane (10 mL) was added and stirring was continued for 1 h. Water (25 mL) was added and the reaction mixture was acidified with 1 M HCl to pH 3. The product was extracted with EtOAc (3×35 mL) and the combined organic layers were washed with brine (2×50 mL), dried over Na₂SO₄, filtered and concentrated. The crude acid (0.125 g, 0.284 mmol) was reacted with amine XT48 (0.146 g, 0.270 mmol) according to general procedure XXA. Purification by flash chromatography (silica gel, MeOH:DCM 0:1 to 1:4) afforded amide XS5 (0.159 g, 66%, 2 steps) as a yellow sticky solid.

¹H NMR (400 MHz, DMSO-d₆) ppm=10.30 (s, 1H), 8.80 (s, 1H), 8.74 (d, J=7.5 Hz, 1H), 8.66 (s, 1H), 8.61-8.58 (m, 1H), 8.44-8.36 (m, 1H), 8.36-8.29 (m, 1H), 8.16 (d, J=6.8 Hz, 1H), 7.90 (d, J=8.8 Hz, 2H), 7.86-7.76 (m, 1H), 7.74-7.62 (m, 1H), 7.60 (d, J=8.6 Hz, 2H), 7.49 (d, J=8.5 Hz, 1H), 7.42-7.34 (m, 2H), 6.72 (d, J=8.8 Hz, 1H), 6.68-6.63 (m, 1H), 5.25-5.18 (m, 2H), 4.46-4.36 (m, 2H), 3.90-3.78 (m, 1H), 3.63 (s, 3H), 3.27-3.19 (m, 2H), 3.15-3.07 (m, 2H), 1.96-1.78 (m, 2H), 1.68-1.49 (m, 2H), 1.38 (s, 9H), 1.31 (d, J=7.0 Hz, 3H), 1.26 (d, J=7.1 Hz, 3H).

MS (ESI⁺) calc. for C₄₁H₅₂ClN₁₂O₉⁺ [M+H]⁺ 891.37, found 891.83.

### (S)-5-(2-Chloro-4-((S)-2-((S)-2-(6-(2,5-dioxo-2,5-dihydro-1H-pyrrol-1-yl) hexanamido)-3-methylbutanamido)propanamido)benzamido)-2-(4-(((2,4-diaminopteridin-6-yl)methyl) amino)benzamido)pentanoic acid (XS6)

To a suspension of methyl ester XS5 (0.159 g, 0.178 mmol) in DMSO (0.16 mL) and MeOH (0.80 mL) was added dropwise NaOH (2.0 M, 0.535 mL, 1.07 mmol) at 10° C. The reaction mixture was allowed to reach RT and was stirred for 15 min. More NaOH (2.0 M, 0.535 mL, 1.07 mmol) was added and after 2 h the reaction mixture was diluted with DMSO (0.32 mL) and MeOH (1.6 mL). The reaction was stirred for 2 h, followed by a final addition of NaOH (2.0 M, 0.535 mL, 1.07 mmol) and stirring for 4 h. The product was then precipitated by the addition of AcOH (1.0 M, 6.0 mL), filtered and washed with water (2.0 mL), MeCN (2.0 mL) and Et₂O (2.0 mL). The crude material was suspended in DCM (5.0 mL) and the mixture was cooled to 0° C. TFA (5.0 mL) was added dropwise and the reaction mixture was stirred for 30 min. The reaction was concentrated and subsequently coevaporated with toluene (10 mL) to yield the crude amine as a yellow oil. The material was dissolved in DMF (6.0 mL), and 6-maleimidohexanoic acid N-hydroxylsuccinimide ester (55.1 mg, 0.179 mmol) and DIPEA (0.125 mL, 0.715 mmol) were added at 0° C. After 15 min, more DIPEA (0.125 mL, 0.715 mmol) was added, followed by the addition of 6-maleimidohexanoic acid N-hydroxylsuccinimide ester (11.0 mg, 0.036 mmol) after 2.5 h. The reaction mixture was concentrated after a total reaction time of 3.5 h and was subsequently coevaporated with toluene (5 mL). The crude was purified by preparative RP-HPLC (water×0.1% TFA/MeCN×0.1% TFA, gradient 20% to 50%). Product fractions were pooled, MeCN was removed by rotary evaporation and the aq. solution was lyophilized to yield maleimide XS6 (22.3 mg, 13%, 3 steps) as a yellow solid.

MS (ESI⁺) calc. for C₄₄H₅₃ClN₁₃O₉⁺ [M+H]⁺ 942.38, found 942.90.

### Preparation of 2-(((S)-4-carboxy-4-(4-(((2,4-diaminopteridin-6-yl)methyl)amino)-2-fluorobenzamido)butyl)carbamoyl)-5-((S)-2-((S)-2-(6-(2,5-dioxo-2,5-dihydro-1H-pyrrol-1-yl)hexanamido)-3-methylbutanamido)propanamido)benzoic acid (XS12)

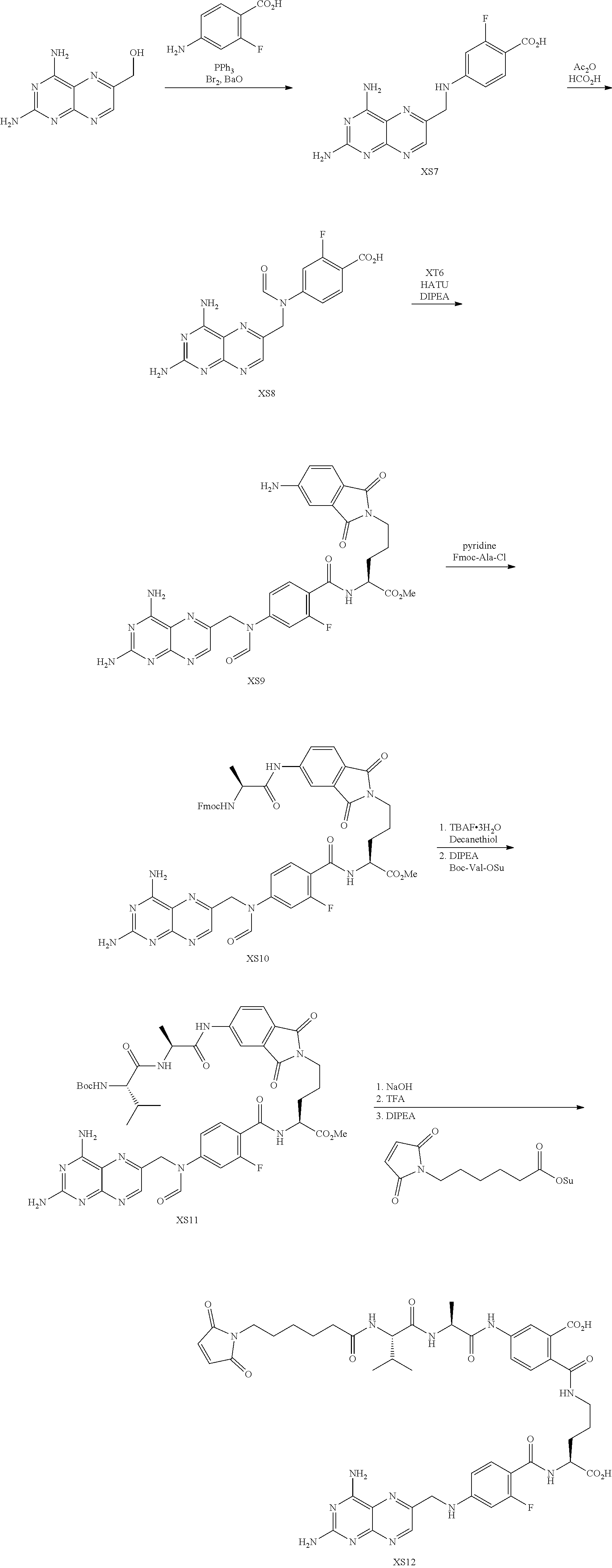

### 4-(((2,4-Diaminopteridin-6-yl)methyl)amino)-2-fluorobenzoic acid (XS7)

Bromine (0.804 mL, 15.6 mmol) was dropwise added to suspension of PPh₃ (4.09 g, 15.6 mmol) in DMA (14 mL) at 0° C. over 10 min. After stirring for 30 min, (2,4-diaminopteridin-6-yl)methanol (1.00 g, 5.20 mmol, prepared as described for XX9) was added and the resulting mixture was stirred for 18 h at RT. Finally, 4-amino-2-fluorobenzoic acid (1.07 g, 6.90 mmol) and BaO (1.11 g, 6.50 mmol) were added and the suspension was heated at 55° C. for 24 h. After cooling to RT, the reaction mixture was poured into DCM:MeOH (150 mL, 29:1) and the solids were collected by filtration. The residue was stirred in water (50 mL), filtrated, stirred in hot MeCN (60 mL) and filtered after cooling to RT, to yield aniline XS7 (1.30 g, 76%) as a brown solid.

MS (ESI⁺) calc. for C₁₄H₁₃FN₇O₂⁺ [M+H]330.11, found 330.35.

### 4-(N-((2,4-Diaminopteridin-6-yl)methyl)formamido)-2-fluorobenzoic acid (XS8)

A solution of acetic anhydride (11.1 mL, 118 mmol) in formic acid (48.3 mL, 1.26 mol) was stirred for 40 min at RT, after which aniline XS7 (1.30 g, 3.94 mmol) was added. The resulting suspension was stirred for 2.5 h at reflux. The reaction mixture was cooled to RT and concentrated. The residue was dissolved in water (88 mL)/aq. NH₄OH (35%, 12.6 mL) and was then heated to 70° C. for 30 min. The mixture was filtered and the filtrate was acidified with AcOH to pH˜5.5. The precipitate was collected by filtration and was subsequently stirred in water/AcOH (55 mL, 10:1) at 50° C. for 45 min. After filtration, the solid was washed with water (2×10 mL), EtOH (2×5 mL) and Et₂O (2×5 mL), and dried under vacuum to yield formamide XS8 (0.788 g, 55%) as a pale-brown powder.

¹H NMR (400 MHz, DMSO-d₆) ppm=8.90 (s, 1H), 8.69 (s, 1H), 7.86 (t, J=8.5 Hz, 1H), 7.72 (br s, 1H), 7.55 (dd, J=12.6, 2.0 Hz, 1H), 7.47-7.36 (m, 2H), 6.75 (br s, 2H), 5.23 (s, 2H).

¹³C NMR (100 MHz, DMSO-d₆) ppm=165.1, 163.2, 163.1, 160.8, 155.3, 150.0, 146.8, 143.8, 133.3, 121.6, 117.3, 116.3, 110.2, 109.9, 46.5.

MS (ESI⁺) calc. for C₁₅H₁₃FN₇O₃⁺ [M+H]358.11, found 358.38.

### Methyl (S)-5-(5-amino-1,3-dioxoisoindolin-2-yl)-2-(4-(N-((2,4-diaminopteridin-6-yl)methyl) formamido)-2-fluorobenzamido)pentanoate (XS9)

Acid XS8 (760 mg, 2.13 mmol) was reacted with amine XT6 (871 mg, 2.66 mmol) according to general procedure XXA. Purification by flash chromatography (silica gel, MeOH:DCM 0:1 to 1:3) afforded amide XS9 (406 mg, 30%) as a yellow solid.

¹H NMR (400 MHz, DMSO-d₆) ppm=8.83 (s, 1H), 8.69 (br s, 1H), 8.59 (d, J=7.3 Hz, 2H), 7.72 (br s, 1H), 7.58 (s, 1H), 7.57-7.53 (m, 1H), 7.46 (d, J=8.1 Hz, 1H), 7.42 (dd, J=8.4, 1.8 Hz, 1H), 6.91 (d, J=2.0 Hz, 1H), 6.78 (dd, J=8.3, 2.0 Hz, 2H), 6.70 (br s, 1H), 6.45 (br s, 2H), 5.23 (s, 2H), 4.44-4.37 (m, 1H), 3.62 (s, 3H), 3.50 (t, J=6.3 Hz, 2H), 1.80-1.68 (m, 2H), 1.66-1.57 (m, 2H).

MS (ESI⁺) calc. for C₂₉H₂₈FN₁₀O₆⁺ [M+H]⁺ 631.22, found 631.66.

### Methyl (S)-5-(5-((S)-2-((((9H-fluoren-9-yl)methoxy)carbonyl)amino)propanamido)-1,3-dioxoisoindolin-2-yl)-2-(4-(N-((2,4-diaminopteridin-6-yl)methyl)formamido)-2-fluorobenzamido)pentanoate (XS10)

Aniline XS9 (315 mg, 0.500 mmol) was reacted with Fmoc-Ala-Cl according to the procedure for XT11. After concentration of the quenched reaction mixture and coevaporation with MeOH:toluene (30 mL, 1:2), the material was purified by flash chromatography (silica gel, MeOH:DCM 0:1 to 1:4) to yield amide XS10 (0.352 g, 76%) as a yellow solid.

¹H NMR (400 MHz, DMSO-d₆) ppm=10.68 (s, 1H), 8.83 (s, 1H), 8.66 (s, 1H), 8.59 (d, J=7.1 Hz, 1H), 8.20 (s, 1H), 7.93-7.88 (m, 3H), 7.81 (d, J=8.3 Hz, 2H), 7.74 (t, J=7.1 Hz, 2H), 7.70-7.64 (m, 1H), 7.60-7.57 (m, 1H), 7.56-7.53 (m, 1H), 7.45-7.37 (m, 4H), 7.37-7.31 (m, 2H), 6.67 (br s, 2H), 5.22 (s, 2H), 4.46-4.37 (m, 1H), 4.34-4.27 (m, 3H), 4.23 (t, J=6.8 Hz, 1H), 3.62 (s, 3H), 3.59-3.54 (m, 2H), 1.83-1.72 (m, 2H), 1.72-1.61 (m, J=8.5 Hz, 2H), 1.35 (d, J=7.1 Hz, 3H).

MS (ESI⁺) calc. for C₄₇H₄₃FN₁₁O₉⁺ [M+H]⁺ 924.32, found 924.86.

### Methyl (S)-5-(5-((S)-2-((S)-2-((tert-butoxycarbonyl)amino)-3-methylbutanamido) propanamido)-1,3-dioxoisoindolin-2-yl)-2-(4-(N-((2,4-diaminopteridin-6-yl)methyl) formamido)-2-fluorobenzamido)pentanoate (XS11)

Fmoc-protected amine XS10 (0.150 g, 0.162 mmol) was deprotected with TBAF·3H₂O and decanethiol, and subsequently reacted with Boc-Val-OSu, analogous to the conversion of XT11 to XT12. Purification by flash chromatography (silica gel, MeOH:DCM 0:1 to 1:4) afforded amide XS11 (85 mg, 46%, 2 steps) as a yellow film.

MS (ESI⁺) calc. for C₄₂H₅₀FN₁₂O₁₀⁺ [M+H]⁺ 901.38, found 901.90.

### 2-(((S)-4-Carboxy-4-(4-(((2,4-diaminopteridin-6-yl)methyl)amino)-2-fluorobenzamido) butyl)carbamoyl)-5-((S)-2-((S)-2-(6-(2,5-dioxo-2,5-dihydro-1H-pyrrol-1-yl)hexanamido)-3-methylbutanamido)propanamido)benzoic acid (XS12)

To a suspension of carbamate XS11 (85 mg, 0.094 mmol) in DCM (3.0 mL) at 0° C. was dropwise added TFA (3.0 mL). The reaction mixture was allowed to reach RT and stirred for 1 h. The solution was concentrated and coevaporated with DCM:toluene (7 mL, 2:5) and toluene (5 mL), to yield the crude amine as a dark-yellow film. The material was dissolved in MeOH/DMSO (0.60 mL, 5:1) and aq. NaOH (2.0 M, 0.285 mL, 0.569 mmol) was dropwise added at 10° C. The cooling bath was removed, and the mixture was stirred for 1 h at RT. More aq. NaOH (0.285 mL, 0.569 mmol) was added and the mixture was stirred for 5 h. The reaction was cooled to 0° C., and aq. AcOH (1.0 M, 1.0 mL) was added, followed by water (2.0 mL). The solids were filtered off and then washed with water (2 mL) and MeCN (2 mL), before being dissolved in DMF (15 mL). To this solution was then added 6-maleimidohexanoic acid N-hydroxylsuccinimide ester (0.035 g, 0.11 mmol) and DIPEA (0.100 mL, 0.572 mmol) at RT. The reaction was stirred for 4 h and was then concentrated, coevaporated with DCM:toluene (10 mL, 1:4) and purified by preparative RP-HPLC (water×0.10% TFA/MeCN×0.1% TFA, gradient 20% to 50%). Product fractions were pooled, MeCN was removed by rotary evaporation and the aq. solution was lyophilized to yield maleimide XS12 (19.6 mg, 21%, 3 steps) as a pale yellow solid.

¹H NMR (400 MHz, DMSO-d₆) ppm=13.50-12.42 (m, 2H), 9.29 (br s, 2H), 8.84 (s, 1H), 8.59 (br s, 1H), 8.27-8.18 (m, 2H), 7.97 (d, J=2.1 Hz, 1H), 7.86-7.65 (m, 4H), 7.63-7.51 (m, 1H), 7.37 (d, J=8.4 Hz, 1H), 7.16 (br s, 1H), 7.00 (s, 2H), 6.63 (dd, J=2.0, 8.6 Hz, 1H), 6.53 (dd, J=1.8, 14.4 Hz, 1H), 4.62 (br s, 2H), 4.43-4.32 (m, 2H), 4.20-4.13 (m, 1H), 3.37-3.33 (m, 2H), 3.21-3.14 (m, 2H), 2.23-2.06 (m, 2H), 2.01-1.89 (m, 2H), 1.88 (br s, 1H), 1.84-1.71 (m, 1H), 1.64-1.53 (m, 2H), 1.53-1.43 (m, 4H), 1.33-1.30 (m, 2H), 1.24-1.13 (m, 3H), 0.87 (d, J=6.6 Hz, 3H), 0.82 (d, J=6.8 Hz, 3H).

MS (ESI⁺) calc. for C₄₅H₅₃FN₁₃O₁₁⁺ [M+H]⁺ 970.40, found 971.00.

### Preparation of (S)-2-(4-(((2,4-diaminopteridin-6-yl)methyl)amino)benzamido)-5-(4-hydroxybenzamido)pentanoic acid (XS14)

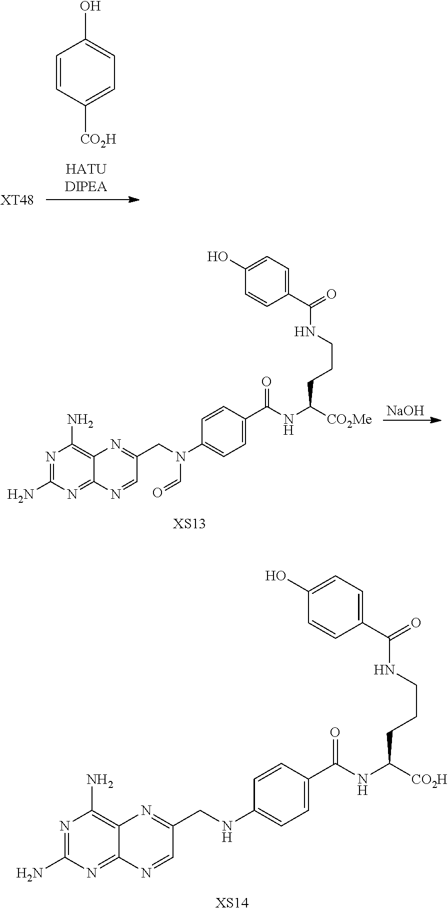

### Methyl (S)-2-(4-(N-((2,4-diaminopteridin-6-yl)methyl)formamido)benzamido)-5-(4-hydroxybenzamido)pentanoate (XS13)

Amine XT48 (97 mg, 0.18 mmol) was reacted with 4-hydroxybenzoic acid according to general procedure XXA. After concentration of the reaction mixture, the crude was coevaporated with DCM/toluene (4 mL, 1:1), toluene (5 mL) and MeCN (2×2 mL). The residue was stirred in MeOH/MeCN (3 mL, 1:1) for 5 min, and was then filtered. The solids were washed with MeCN (2 mL) and Et₂O (2 mL) and, after drying under vacuum, afforded amide XS13 (44 mg, 44%) as a yellow solid.

MS (ESI⁺) calc. for C₂₈H₃₀N₉O₆⁺ [M+H]⁺ 588.23, found 588.66.

### (S)-2-(4-(((2,4-Diaminopteridin-6-yl)methyl)amino)benzamido)-5-(4-hydroxybenzamido) pentanoic acid (XS14)

A suspension of ester XS13 (44 mg, 0.074 mmol) in MeOH/DMSO (0.48 mL, 5:1) was cooled to 10° C. and aq. NaOH (2.0 M, 0.223 mL, 0.446 mmol) was then added dropwise. The reaction mixture was stirred at RT for 5 h. The mixture was then cooled to 0° C. and aq. AcOH (1.0 M, 0.6 mL) was added, followed by water (1.3 mL). The suspension was stirred for 20 min. Solids were collected by filtration, washed with water (1 mL), MeCN (1.0 mL) and Et₂O (2×1 mL), and dried on high vacuum. The residue was purified by preparative RP-HPLC (water×0.1% TFA/MeCN×0.1% TFA, gradient 10% to 55%). Product fractions were pooled, MeCN was removed by rotary evaporation and the aq. solution was lyophilized to yield acid XS14 (21.2 mg, 52%) as a pale yellow solid.

¹H NMR (400 MHz, DMSO-d₆) ppm=13.51-12.76 (m, 1H), 12.57-12.27 (m, 1H), 9.87 (s, 1H), 9.07-8.92 (m, 2H), 8.75 (s, 1H), 8.16-8.09 (m, 2H), 7.65 (d, J=8.7 Hz, 4H), 7.59-7.37 (m, 1H), 6.82 (t, J=5.8 Hz, 1H), 6.73-6.64 (m, 4H), 4.58-4.48 (m, 2H), 4.33-4.23 (m, 1H), 3.20-3.14 (m, 2H), 1.82-1.63 (m, 2H), 1.62-1.40 (m, 2H).

MS (ESI⁺) calc. for C₂₆H₂₈N₉O₅⁺ [M+H]⁺ 546.22, found 546.63.

### Preparation of (S)-5-(5-aminothiophene-2-carboxamido)-2-(4-(((2,4-diaminopteridin-6-yl)methyl)amino)benzamido)pentanoic acid (XS16)

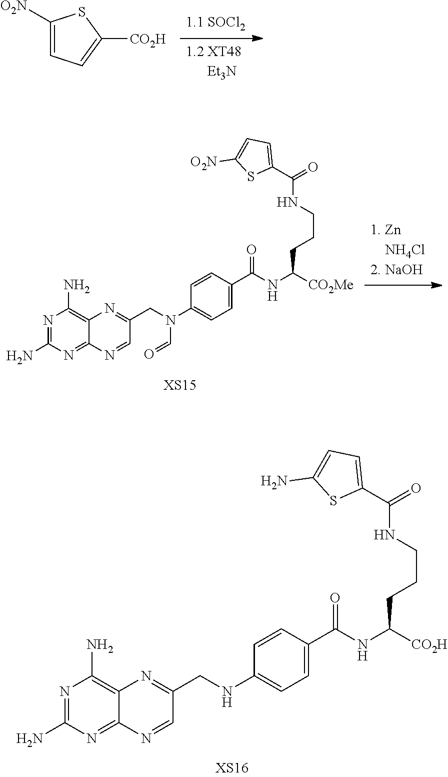 [SMILES: O=C(O)c1ccc([N+](=O)[O-])s1 ; COC(=O)[C@H](CCCNC(=O)c1ccc([N+](=O)[O-])s1)NC(=O)c1ccc(N(C=O)Cc2cnc3nc(N)nc(N)c3n2)cc1 ; Nc1nc(N)c2nc(CNc3ccc(C(=O)N[C@@H](CCCNC(=O)c4ccc(N)s4)C(=O)O)cc3)cnc2n1]

### Methyl (S)-2-(4-(N-((2,4-diaminopteridin-6-yl)methyl)formamido)benzamido)-5-(5-nitrothiophene-2-carboxamido)pentanoate (XS15)

A solution of 5-nitrothiophene-2-carboxylic acid (67 mg, 0.389 mmol) in thionyl chloride (0.621 mL, 8.51 mmol) was heated at reflux for 4 h. The reaction mixture was cooled to RT and coevaporated with heptane (2×1 mL). The residue was dissolved in DMF (0.48 mL) and added to a stirring solution of amine XT48 (0.200 g, 0.370 mmol) and Et₃N (0.103 mL, 0.740 mmol) in DMF (0.48 mL) at 0° C. The reaction mixture was stirred at RT for 20 min and was then quenched with MeOH (5 mL). The mixture was concentrated and coevaporated with toluene (2×5 mL). The residue was dissolved in MeOH (10 mL) and MeCN (20 mL) was added to induce precipitation. MeOH was partly evaporated and the remaining solution was stored at 4° C. overnight. Solids were collected by filtration and washed with MeCN (1 mL) and Et₂O (2 mL). The filtrate was concentrated and the residue was purified by flash chromatography (silica gel, MeOH:DCM 0:1 to 1:4). The isolated product was combined with the residue, to yield amide XS15 (0.122 g, 53%).

¹H NMR (400 MHz, DMSO-d₆) ppm=8.98 (t, J=5.6 Hz, 1H), 8.80 (s, 1H), 8.76-8.70 (m, 1H), 8.65 (s, 1H), 8.13 (d, J=4.4 Hz, 1H), 7.89 (d, J=8.6 Hz, 2H), 7.78 (d, J=4.4 Hz, 1H), 7.64 (br s, 1H), 7.59 (d, J=8.6 Hz, 2H), 7.32 (br s, 1H), 6.61 (br s, 2H), 5.22 (s, 2H), 4.49-4.42 (m, 1H), 3.63 (s, 3H), 3.30-3.26 (m, 2H), 1.89-1.74 (m, 2H), 1.71-1.53 (m, 2H).

MS (ESI⁺) calc. for C₂₆H₂₇N₁₀O₇S⁺ [M+H]⁺ 623.18, found 623.52.

### (S)-5-(5-Aminothiophene-2-carboxamido)-2-(4-(((2,4-diaminopteridin-6-yl)methyl)amino) benzamido)pentanoic acid (XS16)

Amide XS15 (0.122 g, 0.196 mmol) was dissolved in DMF (2.0 mL)/aq. NH₄Cl (7.2 M, 0.383 mL, 2.74 mmol). Zinc powder (0.128 g, 1.96 mmol) was added at RT and the reaction mixture was stirred for 3 h. The reaction mixture was filtered over Celite, and the filtrate was stirred under air for 18 h. The mixture was concentrated and coevaporated with MeCN (2 mL). The residue was suspended in MeCN (10 mL) and the solid was collected by filtration. Washing of the solid with MeCN (1 mL) and Et₂O (2 mL), followed by drying under vacuum afforded a brown solid. The material was suspended in MeOH/DMSO (0.48 mL, 5:1), NaOH (2.0M, 0.187 mL, 0.375 mmol) was added dropwise and the reaction mixture was stirred for 5 h at RT. Aq. AcOH (1.0 M, 0.40 mL) was added and the mixture was stirred for 15 min. Water (1.6 mL) was added and after stirring for 30 min, the mixture was stored at 4° C. overnight. The precipitate was collected by filtration and the solid was washed with water (0.5 mL), MeCN (0.5 mL) and Et₂O (1 mL). Purification by preparative RP-HPLC (water×0.1% TFA/MeCN×0.1% TFA, gradient 10% to 25%). Product fractions were pooled, MeCN was removed by rotary evaporation and the aq. solution was lyophilized to yield acid XS16 (4.7 mg, 4%, 2 steps) as a pale yellow fluffy solid.

¹H NMR (400 MHz, DMSO-d6) ppm=12.46 (br s, 1H), 9.29-9.00 (m, 2H), 8.83 (s, 1H), 8.18 (d, J=7.6 Hz, 1H), 7.87 (t, J=5.7 Hz, 1H), 7.74 (d, J=8.8 Hz, 2H), 7.70 (br s, 1H), 7.64-7.45 (m, 1H), 7.25 (d, J=4.0 Hz, 1H), 6.89 (br s, 1H), 6.74 (d, J=8.8 Hz, 2H), 6.53 (br s, 1H), 6.29-6.01 (m, 1H), 5.79 (d, J=4.0 Hz, 1H), 4.61 (br s, 2H), 4.38-4.29 (m, 1H), 3.23-3.10 (m, 2H), 1.87-1.66 (m, 2H), 1.63-1.44 (m, 2H).

MS (ESI⁺) calc. for C₂₄H₂₇N₁₀O₄S⁺ [M+H]⁺ 551.19, found 551.37.

### Preparation of methyl 5-((S)-2-((S)-2-((tert-butoxycarbonyl)amino)-3-methylbutanamido) propanamido)-2-ethynylbenzoate (XS19)

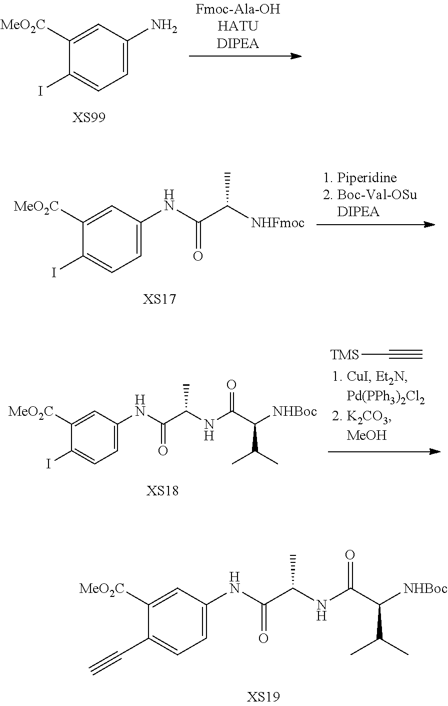

### Methyl (S)-5-(2-((((9H-fluoren-9-yl)methoxy)carbonyl)amino)propanamido)-2-iodobenzoate (XS17)

Amine XS99 (0.800 g, 2.89 mmol, prepared as described in: Ozaki et al, Tetrahedron, 2017, 73, 7177-7184) and Fmoc-Ala-OH (0.944 g, 3.03 mmol) were dissolved in DMF (9.6 mL). DIPEA (2.02 mL, 11.6 mmol) was added, followed by portion wise addition of HATU (1.59 g, 4.18 mmol) during 1 h. The reaction mixture was then stirred for 1 h at RT. The reaction mixture was concentrated and taken up in EtOAc (50 mL). The organic layer was washed with aq. KHSO₄ (0.5 M, 2×25 mL), sat. aq. NaHCO₃ (25 mL) and brine (100 mL), dried over Na₂SO₄ and concentrated. The crude product was purified by flash chromatography (silica gel, EtOAc:DCM 0:1 to 30:70) to yield amide XS17 (1.17 g, 71%) as a pale yellow solid.

¹H NMR (400 MHz, CDCl₃) ppm=8.48 (br s, 1H), 7.99-7.91 (m, 1H), 7.86 (d, J=8.6 Hz, 1H), 7.75 (d, J=7.5 Hz, 2H), 7.60-7.51 (m, 2H), 7.43-7.32 (m, 3H), 7.30-7.23 (m, 2H), 5.30 (br s, 1H), 4.49 (d, J=6.6 Hz, 2H), 4.36 (br s, 1H), 4.21 (t, J=6.7 Hz, 1H), 3.89 (s, 3H), 1.45 (d, J=6.9 Hz, 3H).

¹³C NMR (100 MHz, CDCl₃) ppm=170.4, 166.4, 143.5, 143.4, 141.7, 141.3, 137.8, 135.3, 127.9, 127.2, 127.1, 124.9, 124.0, 122.2, 120.1, 87.3, 67.4, 52.6, 47.1, 17.5.

MS (ESI⁺) calc. for C₂₆H₂₄IN₂O₅⁺ [M+H]⁺ 571.07, found 571.27.

### Methyl 5-((S)-2-((S)-2-((tert-butoxycarbonyl)amino)-3-methylbutanamido) propanamido)-2-iodobenzoate (XS18)

Piperidine (0.989 mL, 9.99 mmol) was added to a stirring solution of amide XS17 (1.14 g, 2.00 mmol) in DMF (10 mL). After stirring for 15 min, the reaction mixture was concentrated and coevaporated with toluene (2×20 mL) and dried under vacuum. The crude product was purified by flash chromatography (silica gel, MeOH:DCM 0:1 to 20:80) to yield the deprotected product (0.740 g, quant). The product and Boc-Val-OSu (0.735 g, 2.34 mmol) were dissolved in DMF (9.7 mL). DIPEA (0.928 mL, 5.31 mmol) was added at RT and the reaction mixture was stirred overnight. The reaction mixture was added to water (100 mL), and the water layer was extracted with EtOAc:heptane (1:1, 3×50 mL). The combined organic layers were washed with water (2×75 mL) and ice-cold brine (2×100 mL), dried over Na₂SO₄ and concentrated. The crude product was purified by flash chromatography (silica gel, EtOAc:heptane 20:80 to 70:30) to yield amide XS18 (0.790 g, 68%) as an off-white solid.

¹H NMR (400 MHz, CDCl₃) ppm=9.02 (br s, 1H), 8.09 (br s, 1H), 7.88 (d, J=8.6 Hz, 1H), 7.60 (d, J=7.3 Hz, 1H), 6.55 (d, J=7.0 Hz, 1H), 5.07-4.92 (m, 1H), 4.67 (quint, J=7.2 Hz, 1H), 3.96-3.92 (m, 1H), 3.91 (s, 3H), 2.27-2.16 (m, 1H), 1.48 (d, J=8.1 Hz, 3H), 1.43 (s, 9H), 1.01 (d, J=6.9 Hz, 3H), 0.96 (d, J=6.9 Hz, 3H).

¹³C NMR (100 MHz, CDCl₃) ppm=171.9, 170.3, 166.5, 141.6, 138.3, 135.1, 124.0, 122.2, 87.0, 81.1, 60.9, 52.5, 49.7, 30.1, 28.2, 19.4, 17.7, 17.5.

MS (ESI⁺) calc. for C₂₁H₃₁IN₃O₆⁺ [M+H]⁺ 548.13, found 548.32.

### Methyl 5-((S)-2-((S)-2-((tert-butoxycarbonyl)amino)-3-methylbutanamido) propanamido)-2-ethynylbenzoate (XS19)

A microwave vial was charged with iodoarene XS18 (0.720 g, 1.32 mmol), Pd(PPh₃)₂C₁₂ (18 mg, 0.026 mmol) and Cu(I)I (13 mg, 0.066 mmol). The vial was purged with N₂ and DMF (4.5 mL), ethynyltrimethylsilane (0.273 mL, 1.97 mmol) and Et₃N (0.183 mL, 1.32 mmol) were added sequentially. The reaction mixture was heated in the microwave at 80° C. for 3.5 h. The reaction mixture was concentrated and coevaporated with toluene (10 mL). The crude product was purified by flash chromatography (silica gel, EtOAc:DCM 0:1 to 1:1) to yield the protected alkyne (0.466, 68%) as a yellow solid. A portion of the intermediate (0.450 g, 0.869 mmol) was dissolved in MeOH (4.4 mL) and K₂CO₃ (12 mg, 0.087 mmol) was added. The reaction mixture was stirred at RT for 1 h. The reaction mixture was then diluted with EtOAc (25 mL) and was subsequently washed with sat. aq. NaHCO₃ (2×20 mL), water (20 mL) and brine (20 mL), dried over Na₂SO₄, filtered, and concentrated, to yield alkyne XS19 (0.365 g, 94%) as a yellow film.

¹H NMR (400 MHz, CDCl₃) ppm=9.06 (br s, 1H), 8.16 (br s, 1H), 7.94 (br d, J=8.4 Hz, 1H), 7.57 (d, J=8.5 Hz, 1H), 6.51 (d, J=6.8 Hz, 1H), 5.00 (br s, 1H), 4.69 (quint, J=7.2 Hz, 1H), 3.98-3.92 (m, 1H), 3.91 (s, 3H), 3.34 (s, 1H), 2.28-2.18 (m, 1H), 1.49 (d, J=7.1 Hz, 3H), 1.43 (s, 9H), 1.01 (d, J=6.9 Hz, 3H), 0.96 (d, J=6.9 Hz, 3H).

¹³C NMR (100 MHz, CDCl₃) ppm=170.3, 166.0, 138.4, 135.8, 133.0, 122.7, 121.2, 117.9, 81.4, 60.9, 52.2, 49.6, 30.1, 28.2, 19.4, 17.7, 17.5.

MS (ESI⁺) calc. for C₂₃H₃₂N₃O₆⁺ [M+H]⁺ 446.23, found 446.35.

### Preparation of 2-(1-((S)-4-carboxy-4-(4-(2-(2,4-diaminopteridin-6-yl)ethyl)benzamido) butyl)-1H-1,2,3-triazol-4-yl)-5-((S)-2-((S)-2-(6-(2,5-dioxo-2,5-dihydro-1H-pyrrol-1-yl) hexanamido)-3-methylbutanamido)propanamido)benzoic acid (XS23)

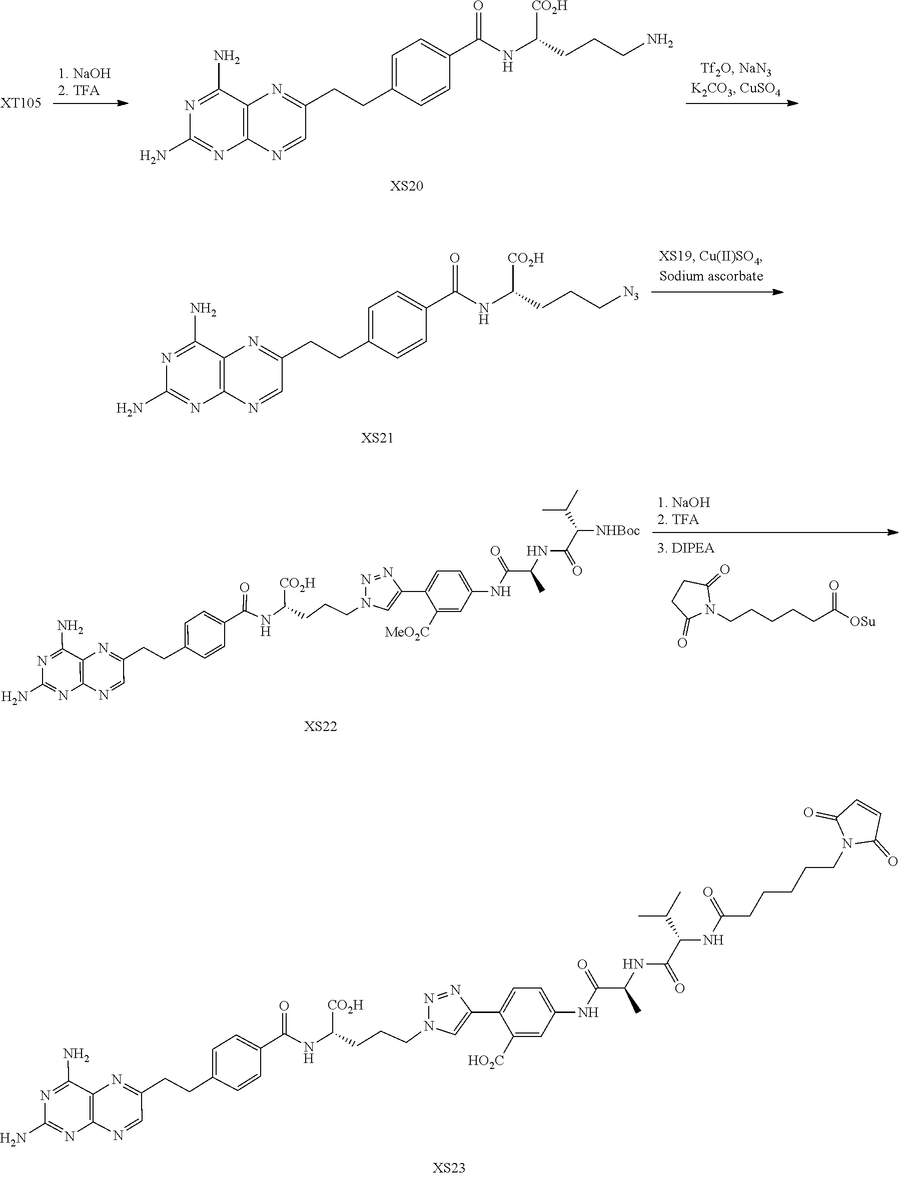

### (S)-5-((tert-Butoxycarbonyl)amino)-2-(4-(2-(2,4-diaminopteridin-6-yl)ethyl)benzamido) pentanoic acid (XS20)

Ester XT105 (0.300 g, 0.557 mmol) was suspended in DMSO (0.33 mL) and MeOH (1.65 mL). The mixture was cooled to 10° C. and 2 M NaOH (2.23 mL, 4.46 mmol) was added dropwise. The reaction mixture was allowed to reach RT and stirred for 5 h. The reaction mixture was cooled to 0° C. and quenched with 1 M aq. AcOH (6 mL), and diluted with water (10 mL). The mixture was warmed to RT and was subsequently filtered. The residue was washed with MeCN (5 mL) and Et₂O (2×5 mL), to yield the crude acid. The material was dissolved in ice-cold DCM (5 mL). TFA (5 mL) was added and the mixture was stirred for 15 min at 0° C. The reaction mixture was concentrated and coevaporated with toluene (2×10 mL), to yield amine XS20 (0.292 g, quant, 2 steps) as a brown oil.

¹H NMR (400 MHz, DMSO-d₆) ppm=12.99-12.41 (m, 1H), 9.14-8.89 (m, 2H), 8.71 (s, 1H), 8.56 (d, J=7.9 Hz, 1H), 8.46-7.90 (m, 2H), 7.82 (d, J=8.3 Hz, 2H), 7.76-7.67 (m, 2H), 7.36 (d, J=8.3 Hz, 2H), 4.47-4.33 (m, 1H), 3.28-3.17 (m, 4H), 2.89-2.74 (m, 2H), 1.96-1.85 (m, 1H), 1.85-1.71 (m, 1H), 1.71-1.56 (m, 2H).

¹³C NMR (100 MHz, DMSO-d₆) ppm=173.9, 166.8, 163.3, 158.9, 158.6, 153.1, 150.6, 145.1, 132.2, 128.8, 128.1, 122.3, 52.4, 39.0, 35.8, 34.0, 28.0, 24.6.

MS (ESI⁺) calc. for C₂₀H₂₅N₈O₃⁺ [M+H]⁺ 425.20, found 425.41.

### (S)-5-Azido-2-(4-(2-(2,4-diaminopteridin-6-yl)ethyl)benzamido)pentanoic acid (XS21)

To an ice-cold solution of NaN₃ (0.361 g, 5.56 mmol) in water (0.95 mL)/DCM (1.6 mL) was dropwise added triflic anhydride (0.188 mL, 1.11 mmol). The resulting mixture was stirred for 4 h at 0° C. The organic layer was separated and the aq. phase was extracted with DCM (2×1 mL). The combined organic layers were washed with 1 M Na₂CO₃ (1 mL), and added to a RT solution of K₂CO₃ (0.123 g, 0.890 mmol) and CuSO₄·5H₂O (2.8 mg, 0.011 mmol) and amine XS20 (0.236 g, 0.556 mmol) in water (1.8 mL)/MeOH (3.7 mL). The resulting mixture was stirred overnight at RT. The reaction mixture was diluted with water and acidified with AcOH (1.0 M) to pH˜5. The mixture was gently heated to cause precipitation and cooled down to RT. Solids were collected by filtration and washed with water (1 mL), MeCN (1 mL) and Et₂O (5 mL), and dried under vacuum to yield azide XS21 (0.103 g, 41%) as a green-brown solid.

¹H NMR (400 MHz, DMSO-d₆) ppm=13.60-11.50 (m, 1H), 8.56 (br s, 1H), 8.54-8.49 (m, 1H), 7.79 (d, J=7.9 Hz, 2H), 7.68-7.48 (m, 2H), 7.35 (d, J=8.0 Hz, 2H), 6.53 (br s, 2H), 4.45-4.28 (m, 1H), 3.38-3.33 (m, 2H), 3.15 (br s, 4H), 1.94-1.71 (m, 2H), 1.71-1.54 (m, 2H).

MS (ESI⁺) calc. for C₂₀H₂₃N₁₀O₃⁺ [M+H]⁺ 451.19, found 451.50.

### (S)-5-(4-(4-((S)-2-((S)-2-((tert-butoxycarbonyl)amino)-3-methylbutanamido) propanamido)-2-(methoxycarbonyl)phenyl)-1H-1,2,3-triazol-1-yl)-2-(4-(2-(2,4-diaminopteridin-6-yl)ethyl) benzamido)pentanoic acid (XS22)

A solution of alkyne XS19 (0.070 g, 0.16 mmol) and azide XS21 (0.078 g, 0.17 mmol) in DMF (1.7 mL) was purged with N₂ for 5 min. Cu(II)SO₄ (33 mg, 0.13 mmol) in water (889 μL) and sodium ascorbate (51 mg, 0.26 mmol) in water (942 μL) were then added sequentially. The resulting mixture was stirred at RT for 18 h. The reaction mixture was added to water (25 mL). Aq. AcOH (1.0 M, 0.5 mL) was added and the mixture was stirred for 30 min. The mixture was gently heated to cause precipitation and cooled to RT. Solids were collected by filtration and washed with MeCN (20 mL), EtOAc (2×20 mL) and Et₂O (10 mL), stirred in EtOAc (15 mL), collected by filtration and washed with Et₂O (10 mL). The solids were then dried under vacuum, to yield triazole XS22 (0.103 g, 66%) as a brown solid.

MS (ESI⁺) calc. for C₄₃H₅₄N₁₃O₉⁺ [M+H]⁺ 896.42, found 896.65.

### 2-(1-((S)-4-Carboxy-4-(4-(2-(2,4-diaminopteridin-6-yl)ethyl)benzamido)butyl)-1H-1,2,3-triazol-4-yl)-5-((S)-2-((S)-2-(6-(2,5-dioxo-2,5-dihydro-1H-pyrrol-1-yl)hexanamido)-3-methylbutanamido)propanamido)benzoic acid (XS23)

Ester XS22 (0.100 g, 0.112 mmol) was suspended in THF (2.0 mL) and cooled to 10° C. LiOH (0.4 M, 1.40 mL, 0.558 mmol) was added dropwise and the reaction mixture was stirred for 4.5 h. More LiOH (0.4 M, 0.698 mL, 0.279 mmol) was added and stirring was continued for 1 h. The reaction mixture was cooled to 0° C. and AcOH (96 μL, 1.67 mmol) was added. The mixture was concentrated and coevaporated with toluene (2×5 mL). The crude material was then dissolved in a 0° C. solution of TFA (3.0 mL) in DCM (3.0 mL). The reaction mixture was stirred for 15 min at 0° C. and was subsequently concentrated and coevaporated with toluene (2×5 mL), and dried under vacuum, to yield the crude amine as a brown oil. The material was dissolved in DMF (5.0 mL), and 6-maleimidohexanoic acid N-hydroxylsuccinimide ester (38 mg, 0.12 mmol) and DIPEA (0.117 mL, 0.668 mmol) were added at 0° C. DIPEA (0.117 mL, 0.668 mmol) was added and 6-maleimidohexanoic acid N-hydroxylsuccinimide ester (15 mg, 0.049 mmol) were added after 15 min and 3 h, respectively. After 24 h, the reaction mixture was concentrated to 1 mL, and stirring was continued for 192 h. More DIPEA (0.117 mL, 0.668 mmol) was added and, after a total reaction time of 240 h, the reaction mixture was concentrated and coevaporated with toluene (5 mL). A portion of the crude was purified by preparative RP-HPLC (water×0.1% TFA/MeCN×0.1% TFA, gradient 20% to 40%). Product fractions were pooled, MeCN was removed by rotary evaporation and the aq. solution was lyophilized to yield maleimide XS23 (13.9 mg, 13%, 3 steps) as a pale yellow solid.

¹H NMR (400 MHz, DMSO-d₆) ppm=12.99-12.41 (m, 1H), 9.14-8.89 (m, 2H), 8.71 (s, 1H), 8.56 (d, J=7.9 Hz, 1H), 8.46-7.90 (m, 2H), 7.82 (d, J=8.3 Hz, 2H), 7.76-7.67 (m, 2H), 7.36 (d, J=8.3 Hz, 2H), 4.47-4.33 (m, 1H), 3.28-3.17 (m, 4H), 2.89-2.74 (m, 2H), 1.96-1.85 (m, 1H), 1.85-1.71 (m, 1H), 1.71-1.56 (m, 2H).

MS (ESI⁺) calc. for C₄₇H₅₅N₁₄O₁₀⁺ [M+H]⁺ 975.42, found 975.60.

### Preparation of (S)-2-(4-(((2,4-diaminopteridin-6-yl)methyl)amino)benzamido)-5-(4-(((2-((((4-((2S,5S)-13-(2,5-dioxo-2,5-dihydro-1H-pyrrol-1-yl)-5-methyl-4,7-dioxo-2-(3-ureidopropyl)-8,11-dioxa-3,6-diazatridecanamido)benzyl)oxy)carbonyl)(methyl)amino) ethyl)(methyl)carbamoyl)oxy) benzamido)pentanoic acid (XR4)

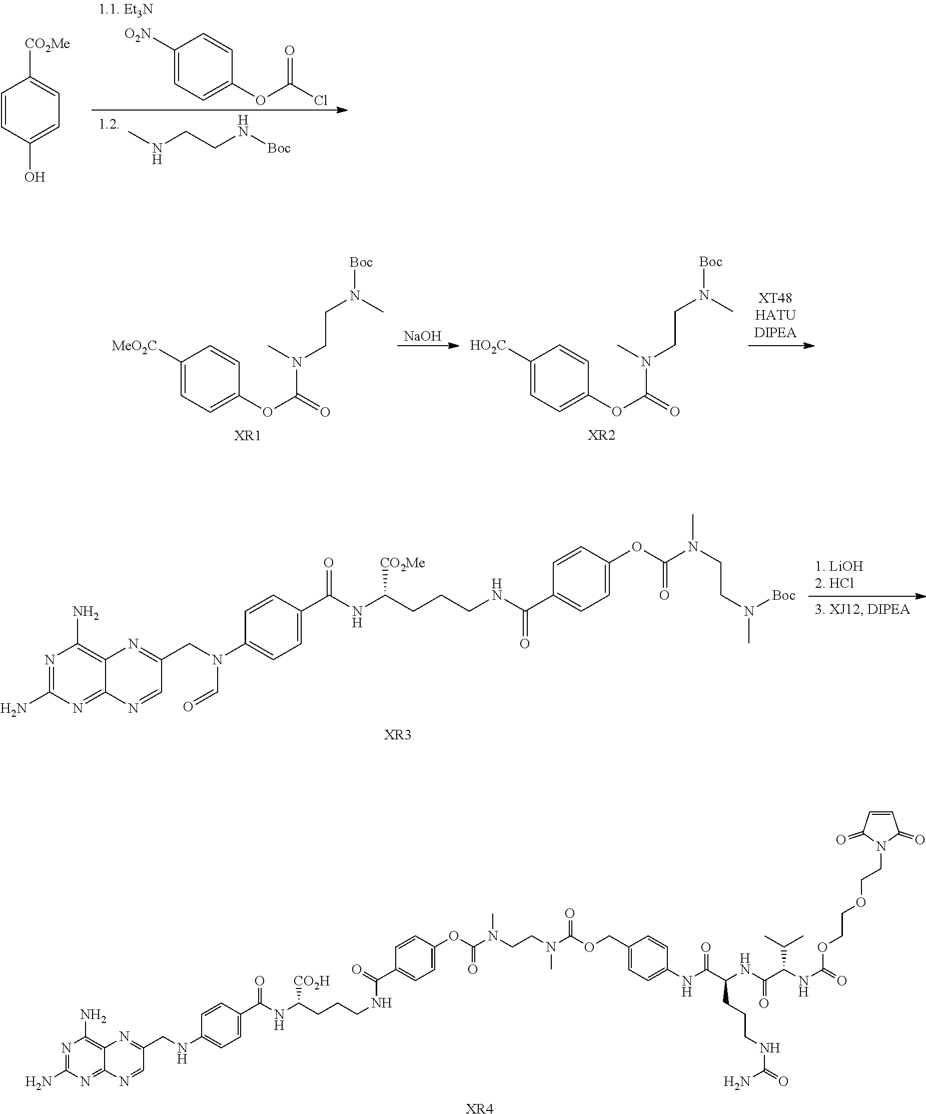

### Methyl 4-(((2-((tert-butoxycarbonyl)(methyl)amino)ethyl)(methyl)carbamoyl)oxy) benzoate (XR1)

To a solution of methyl 4-hydroxybenzoate (1.00 g, 6.57 mmol) in THF (25 mL) was added Et₃N (2 mL, 14.5 mmol) at 0° C. Then, 4-nitrophenyl chloroformate (1.46 g, 7.23 mmol) was added. The resulting suspension was diluted with THF (30 mL) and the mixture was allowed to warm to RT. After 1 h, tert-butyl methyl(2-(methylamino)ethyl)-carbamate (1.24 g, 6.57 mmol) was added and the mixture was stirred for 1 h. The mixture was concentrated and taken up in EtOAc. The EtOAc layer was washed with KHSO₄ (0.5 M, 2×), sat. aq. NaHCO₃ (2×), aq. Na₂CO₃ (1 M) and brine. The organic layer was dried over MgSO₄ and concentrated. The crude product was purified by flash chromatography (silica gel, heptane:EtOAc, 1:0 to 3:7) to give carbamate XR1 (2.07 g, 85%).

¹H NMR (400 MHz, CDCl₃) ppm=8.05 (d, J=8.4 Hz, 2H), 7.19 (d, J=8.6 Hz, 2H), 3.91 (s, 3H), 3.62-3.42 (m, 4H), 3.13-3.05 (s, 3H), 2.93 (m, 3H), 1.44 (m, 9H).

MS (ESI⁺) calc. for C₁₈H₂₇N₂O₆⁺ [M+H]⁺ 367.19, found 367.35.

### 4-(((2-((tert-Butoxycarbonyl)(methyl)amino)ethyl)(methyl)carbamoyl)oxy)benzoic acid (XR2)

Carbamate XR1 (2.07 g, 5.66 mmol) was dissolved in THF/water (1:1, 30 mL) and NaOH (0.293 g, 7.33 mmol) was added. The reaction mixture was heated at 45° C. for 2 h, cooled to RT and acidified with aq. KHSO₄ (0.5 M, ˜50 mL). The reaction mixture was extracted with EtOAc (3×) and the combined organic layers were washed with brine, dried over MgSO₄, filtered and concentrated to give acid XR2.

MS (ESI⁺) calc. for C₁₇H₂₅N₂O₆⁺ [M+H]⁺ 353.17, found 353.27.

### Methyl (S)-5-(4-(((2-((tert-butoxycarbonyl)(methyl)amino)ethyl)(methyl)carbamoyl)oxy) benzamido)-2-(4-(N-((2,4-diaminopteridin-6-yl)methyl)formamido)benzamido)pentanoate (XR3)

Amine XT48 (40 mg, 0.079 mmol) was reacted with acid XR2 (28.0 mg, 0.079 mmol) according to general procedure XXA. Purification by flash chromatography (silica gel, DCM:MeOH, 1:0 to 1:1) afforded amide XR3 (quant).

MS (ESI⁺) calc. for C₃₈H₄₈N₁₁O₉⁺ [M+H]⁺ 802.36, found 802.80.

### (S)-2-(4-(((2,4-Diaminopteridin-6-yl)methyl)amino)benzamido)-5-(4-(((2-((((4-((2S,5S)-13-(2,5-dioxo-2,5-dihydro-1H-pyrrol-1-yl)-5-methyl-4,7-dioxo-2-(3-ureidopropyl)-8,11-dioxa-3,6-diazatridecanamido)benzyl)oxy)carbonyl)(methyl)amino)ethyl)(methyl) carbamoyl)oxy)benzamido)pentanoic acid (XR4)

Ester XR3 (38 mg, 0.047 mmol) was dissolved in water/THF (1:1, 1 mL) and treated with LiOH (5.7 mg, 0.238 mmol) for 3 h, followed by quenching of the reaction with AcOH (10 eq). The mixture was concentrated, coevaporated with toluene (2×) and dried in vacuo.

MS (ESI⁺) calc. for C₃₆H₄₆N₁₁O₈⁺ [M+H]⁺ 760.35, found 760.77.

The obtained intermediate was suspended in HCl/dioxane (2 mL, 4 M) and stirred for 30 min. The reaction mixture was concentrated, coevaporated with chloroform and dried in vacuo. Next, the Boc-deprotected intermediate and XJ12 (45 mg, 0.06 mmol) were dissolved in DMF (1 mL), cooled on an ice bath and DIPEA was added (0.082 mL, 0.47 mmol. After 15 min, the reaction mixture was allowed to warm to RT and stirred for 2 h. The mixture was concentrated and the crude product was purified by preparative RP-HPLC (water×0.1% TFA/MeCN×0.1% TFA). Fractions containing the product were pooled, acetonitrile was evaporated and the remaining solution was lyophilized to yield XR4 (25 mg, 42%).

MS (ESI⁺) calc. for C₅₉H₇₅N₁₇O₁₆⁺ [M+H]⁺ 1277.56, found 1277.12.

### Preparation of 2-(((S)-4-carboxy-4-(4-(((2,4-diaminopteridin-6-yl)methyl)amino)benzamido) butyl)carbamoyl)-5-(((2-((((4-((2S,5S)-13-(2,5-dioxo-2,5-dihydro-1H-pyrrol-1-yl)-5-isopropyl-4,7-dioxo-2-(3-ureidopropyl)-8,11-dioxa-3,6-diazatridecanamido)benzyl)oxy) carbonyl)(methyl)amino)ethyl) (methyl)carbamoyl)oxy)benzoic acid (XR8-p and XR8-m)

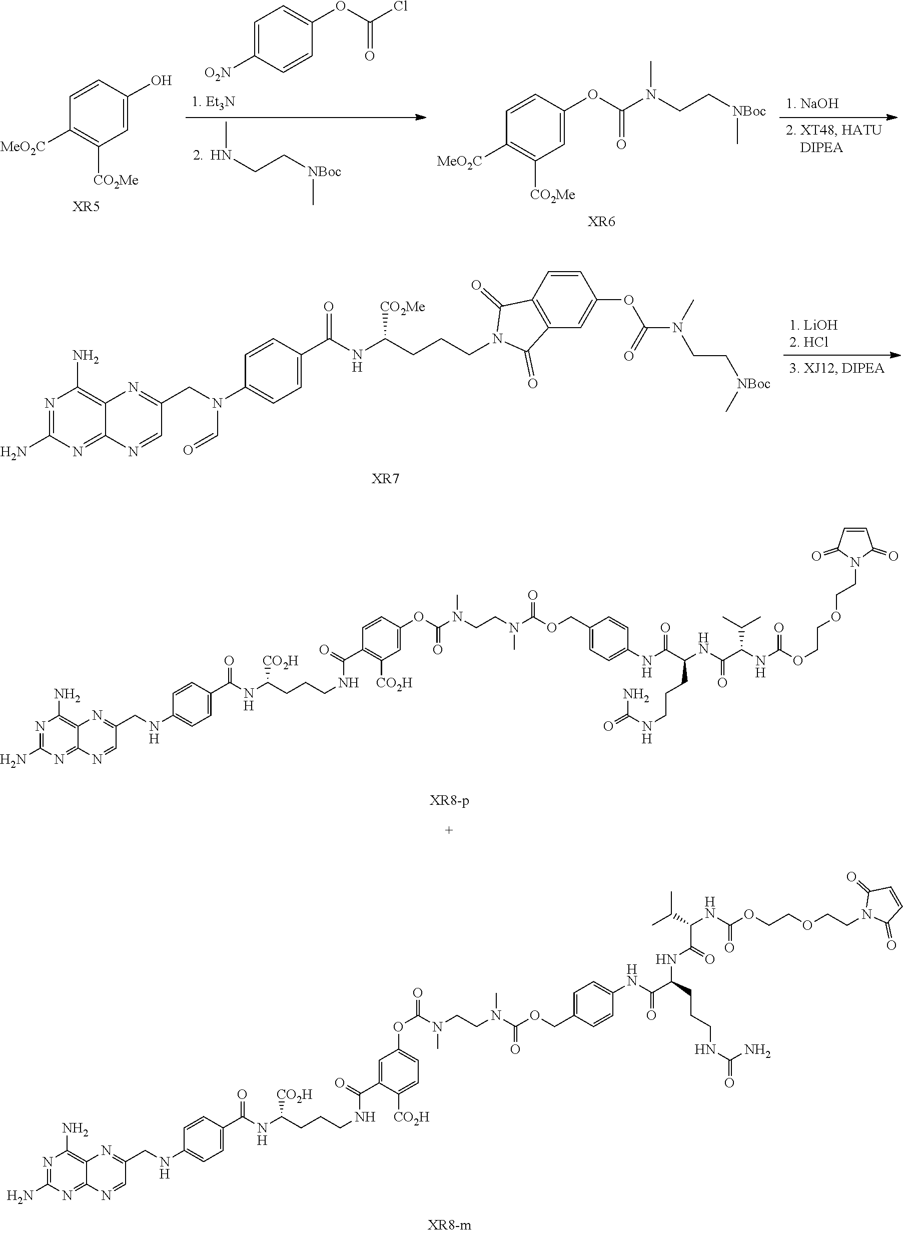

### Dimethyl 4-(((2-((tert-butoxycarbonyl)(methyl)amino)ethyl)(methyl)carbamoyl)oxy) phthalate (XR6)

To a cooled (0° C.) solution of dimethyl 4-hydroxyphthalate (XR5; 500 mg, 2.38 mmol) in DCM (20 mL) was added 4-nitrophenyl chloroformate (527 mg, 2.62 mmol), followed by Et₃N (0.66 mL, 4.76 mmol). The reaction was stirred for 2 h at 0° C. Then, tert-butyl methyl(2-(methylamino)ethyl)carbamate (537 mg, 2.85 mmol) in DCM (2 mL) was added in one portion. The mixture was stirred for 1 h at RT and was then concentrated. The crude product was purified by flash chromatography (silica gel, heptane:EtOAc, 1:0 to 3:7) to give carbamate XR6 (57%, 580 mg).

¹H NMR (400 MHz, CDCl₃) ppm=7.76 (d, J=8.4 Hz, 1H), 7.46 (m, 1H), 7.32 (dd, J=8.5 Hz, 1.9 Hz, 1H), 3.90 (s, 6H), 3.62-3.41 (m, 4H), 3.12 (s, 1.5H, rotamer), 3.05 (s, 1.5H, rotamer), 2.92 (s, 3H), 1.44 (m, 9H). (For clarity, split but partially overlapping singlets originating from carbamate rotamers are reported as singlets).

MS (ESI⁺) calc. for C₂₀H₂₈N₂NaO₈⁺ [M+Na]+447.17, found 447.50.

### Methyl (S)-5-(5-(((2-((tert-butoxycarbonyl)(methyl)amino)ethyl)(methyl)carbamoyl) oxy)-1,3-dioxoisoindolin-2-yl)-2-(4-(N-((2,4-diaminopteridin-6-yl)methyl)formamido)benzamido) pentanoate (XR7)

NaOH (269 mg, 6.71 mmol) in water (4 mL) was added to a solution of carbamate XR6 (570 mg, 1.34 mmol) in THF (5 mL) at RT. After 20 h, the reaction mixture was acidified using aq. KHSO₄ (0.5 M), and the reaction mixture was then extracted with EtOAc (2×25 mL). The combined organic layers were dried over MgSO₄, filtered and concentrated. A portion of the obtained crude diacid (34.7 mg, 0.087 mmol) was reacted with amine XT48 (52 mg, 0.096 mmol) as described in general procedure XXA, using HATU (83 mg, 0.219 mmol) and DIPEA (0.061 mL, 0.350 mmol) in DMF (0.5 mL) and a reaction time of 20 h. After concentration, the crude product was purified by flash chromatography (silica gel, DCM:MeOH, 1:0 to 1:1) to give the phthalimide XR7 (58.2 mg, 80%).

MS (ESI⁺) calc. for C₃₉H₄₆N₁₁O₁₀⁺ [M+H]⁺ 828.34, found 828.83.

### 2-(((S)-4-Carboxy-4-(4-(((2,4-diaminopteridin-6-yl)methyl)amino)benzamido)butyl) carbamoyl)-5-(((2-((((4-((2S,5S)-13-(2,5-dioxo-2,5-dihydro-1H-pyrrol-1-yl)-5-isopropyl-4,7-dioxo-2-(3-ureidopropyl)-8,11-dioxa-3,6-diazatridecanamido)benzyl)oxy)carbonyl) (methyl) amino)ethyl)(methyl) carbamoyl)oxy)benzoic acid (XR8-p and XR8-m)

Phthalimide XR7 (58.2 mg, 0.070 mmol) was dissolved in water/THF (1:1, 1 mL) and treated with LiOH (13.5 mg, 0.56 mmol) for 3 h at RT. The product was precipitated by the addition of AcOH and the solid was subsequently isolated by filtration. The crude material (18 mg, 32%).

MS (ESI⁺) calc. for C₃₇H₄₆N₁₁O₁₀⁺ [M+H]⁺ 804.34, found 804.48.

The saponified intermediate (18 mg) was suspended in HCl/dioxane (4 M, 0.5 mL) and the orange suspension was stirred for 30 min, then concentrated and dried in vacuo. The Boc-deprotected intermediate and XJ12 (16.9 mg, 0.022 mmol) were dissolved in DMF (0.5 mL) followed by the addition of DIPEA (0.023 mL, 0.134 mmol). The mixture was stirred for 2 h at RT and was then concentrated. Purification by preparative RP-HPLC (water×0.10% TFA/MeCN×0.10% TFA) afforded a mixture of para- and meta-opened phthalimide linker-drug XR8-p and XR8-m as a yellow solid.

MS (ESI⁺) calc. for C₆₀H₇₄N₁₇O₁₈⁺ [M+H]⁺ 1320.54, found 1320.74.

### Preparation of (S)-5-(2-(benzyloxy)-4-(((2-((((4-((2S,5S)-13-(2,5-dioxo-2,5-dihydro-1H-pyrrol-1-yl)-5-methyl-4,7-dioxo-2-(3-ureidopropyl)-8,11-dioxa-3,6-diazatridecanamido) benzyl)oxy)carbonyl) (methyl)amino)ethyl)(methyl)carbamoyl)oxy)benzamido)-2-(4-(((2,4-diaminopteridin-6-yl)methyl)amino)benzamido)pentanoic acid (XR12)

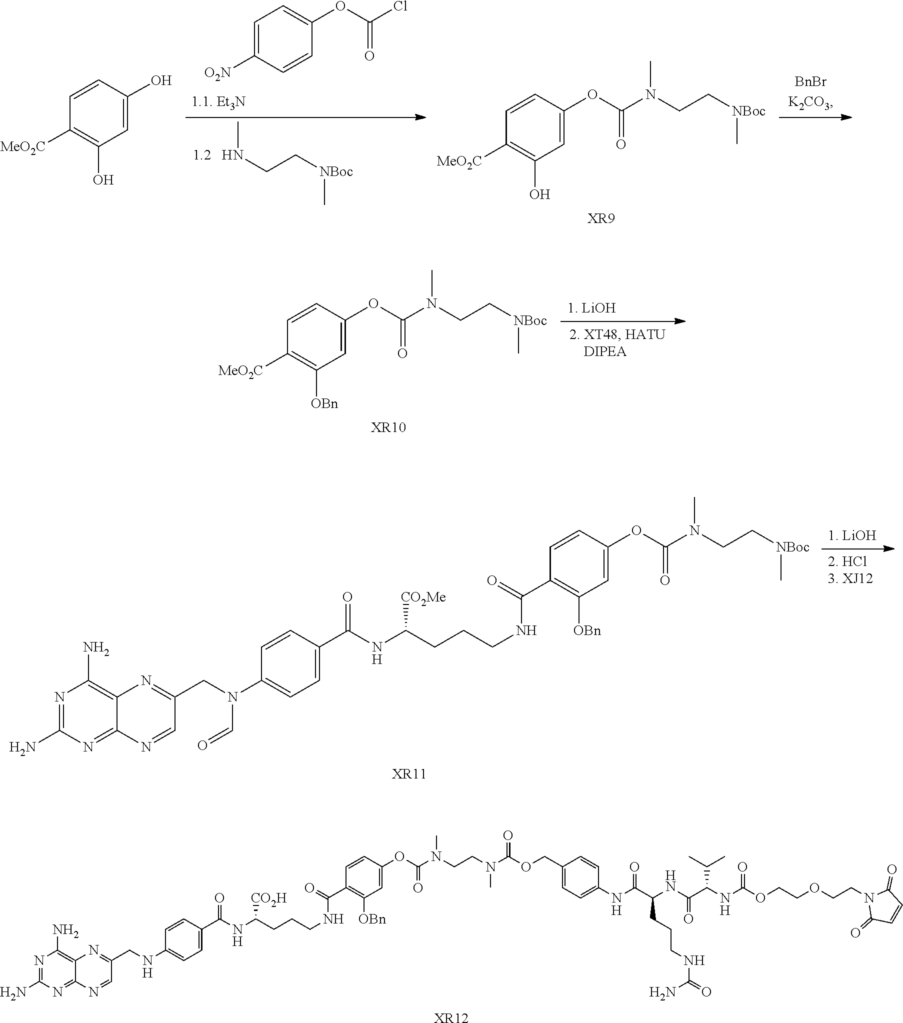

### Methyl 4-(((2-((tert-butoxycarbonyl)(methyl)amino)ethyl)(methyl)carbamoyl)oxy)-2-hydroxybenzoate (XR9)

Et₃N (1.71 mL, 12.3 mmol) and 4-nitrophenyl chloroformate (1.36 g, 6.74 mmol) were sequentially added to a cooled (0° C.) solution of methyl 2,4-dihydroxybenzoate (1.03 g, 6.13 mmol) in THF (50 mL). After 1 h at 0° C., tert-butyl methyl(2-(methylamino)ethyl)-carbamate (1.27 g, 6.74 mmol) in THF (10 mL) was added and the mixture was allowed to stir at RT for 1 h. After concentration of the reaction mixture, the crude was purified by flash chromatography (silica gel, heptane:EtOAc, 1:0 to 1:1) to give carbamate XR9 (1.64 g, 70%).

¹H NMR (400 MHz, CDCl₃) ppm=10.85 (s, 1H), 7.82 (dd, J=8.8 Hz, J=1.8 Hz, 1H), 6.75 (d, J=2.1 Hz, 1H), 6.69 (d, J=8.9 Hz, 1H), 3.94 (s, 3H), 3.61-3.42 (m, 4H), 3.11 (s, 1.5H, rotamer), 3.04 (s, 1.5H, rotamer), 2.92 (s, 3H), 1.45 (s, 9H). (For clarity, split but partially overlapping singlets originating from carbamate rotamers are reported as singlets).

MS (ESI⁺) calc. for C₁₈H₂₇N₂O₇⁺ [M+H]⁺ 383.18, found 383.27.

### Methyl 2-(benzyloxy)-4-(((2-((tert-butoxycarbonyl)(methyl)amino)ethyl)(methyl)carbamoyl) oxy)benzoate (XR10)

BnBr (0.253 mL, 2.13 mmol) and K₂CO₃ (321 mg, 2.32 mmol) were added to a solution of carbamate XR9 (740 mg, 1.935 mmol) in DMF (10 mL) at RT and the mixture was stirred for 16 h. Next, the mixture was concentrated and taken up in EtOAc (˜75 mL). The organic layer was washed with water, aq. KHSO₄ (0.5 M) and brine. The EtOAc layer was dried over MgSO₄, filtered and concentrated. The crude product was purified by flash chromatography twice (1; silica gel, heptane:EtOAc, 1:0 to 1:1, 2; DCM:MeOH, 1:0 to 4:1) to give benzyl ether XR10 (585 mg, 64%).

MS (ESI⁺) calc. for C₂₅H₃₂N₂NaO₇⁺ [M+Na]+495.21, found 495.55.

### Methyl (S)-5-(2-(benzyloxy)-4-(((2-((tert-butoxycarbonyl)(methyl)amino)ethyl)(methyl) carbamoyl)oxy) benzamido)-2-(4-(N-((2,4-diaminopteridin-6-yl)methyl)formamido) benzamido)pentanoate (XR11)

LiOH (148 mg, 6.2 mmol) was added to a solution of XR10 (585 mg, 1.24 mmol) in THF (10 mL) at RT and the mixture was stirred overnight. More LiOH (59.4 mg, 2.48 mmol) was added and the reaction was continued for 24 h. The reaction was acidified with aq. KHSO₄ (0.5 M) and extracted with EtOAc (2×25 mL). The combined organic layers were dried over MgSO₄, filtered and concentrated to give the crude acid (518 mg).

¹H NMR (400 MHz, CDCl₃) ppm=10.55 (br s, 1H), 8.18 (d, J=8.6 Hz, 1H), 7.48-7.37 (m, 5H), 7.15-7.05 (m, 1H), 6.95-6.86 (m, 1H), 5.25 (s, 2H), 3.59-3.54 (m, 1H), 3.52-3.44 (m, 3H), 3.13 (s, 1.3H, rotamer), 3.06 (s, 1.7H, rotamer), 2.92 (s, 3H), 1.45 (s, 9H). (For clarity, split but partially overlapping singlets originating from carbamate rotamers are reported as singlets).

A portion of this material (52.5 mg, 0.115 mmol) was reacted with amine XT48 (57.7 mg, 0.115 mmol), HATU (52.2 mg, 0.137 mmol) and DIPEA (0.123 mL, 0.687 mmol) in DMF (1 mL) according to general procedure XXA. Purification of the crude product by flash chromatography (silica gel, DCM:MeOH, 1:0 to 1:1) afforded amide XR11 (100 mg, 87%).

MS (ESI⁺) calc. for C₄₅H₅₄N₁₁O₁₀⁺ [M+H]⁺ 908.40, found 908.55.

### (S)-5-(2-(Benzyloxy)-4-(((2-((((4-((2S,5S)-13-(2,5-dioxo-2,5-dihydro-1H-pyrrol-1-yl)-5-methyl-4,7-dioxo-2-(3-ureidopropyl)-8,11-dioxa-3,6-diazatridecanamido)benzyl)oxy) carbonyl) (methyl)amino)ethyl)(methyl)carbamoyl)oxy)benzamido)-2-(4-(((2,4-diaminopteridin-6-yl)methyl)amino)benzamido)pentanoic acid (XR12)

Maleimide XR12 was synthesized analogous to the procedure describing the conversion of XR3 to XR4. The product was obtained as a yellow solid.

¹H NMR (400 MHz, DMSO-d₆) ppm=13.10 (br s, 1H), 12.41 (br s, 1H), 11.01 (s, 1H), 9.96 (s, 1H), 9.19 (s, 1H), 9.14 (s, 1H), 8.77 (s, 1H), 8.14-7.96 (m, 5H), 7.68 (d, J=8.7 Hz, 2H), 7.63 (t, J=8.1 Hz, 1H), 7.50 (m, 2H), 7.39 (m, 2H), 7.29-7.08 (m, 6H), 6.94 (s, 2H), 6.92-6.80 (m, 4H), 6.69 (m, 3H), 5.91 (t, J=5.4 Hz, 1H), 5.35 (br s, 2H), 5.09 (m, 2H), 4.94 (m, 2H), 4.55 (s, 2H), 4.39-4.20 (m, 2H), 3.94 (m, 2H), 3.82 (t, J=7.4 Hz, 1H), 3.52-3.36 (m, 11H), 3.17 (m, 2H), 3.00-2.75 (m, 8H), 1.90 (m, 1H), 1.79-1.22 (m, 8H), 0.79 (d, J=6.7 Hz, 3H), 0.75 (d, J=6.7 Hz, 3H).

MS (ESI⁺) calc. for C₆₆H₈₀N₁₇O₁₇⁺ [M+H]⁺ 1382.59, found 1382.81.

### Preparation of 5-((R)-2-((R)-2-((tert-butoxycarbonyl)amino)-3-methylbutanamido)-propanamido)thiophene-2-carboxylic acid (XR23)

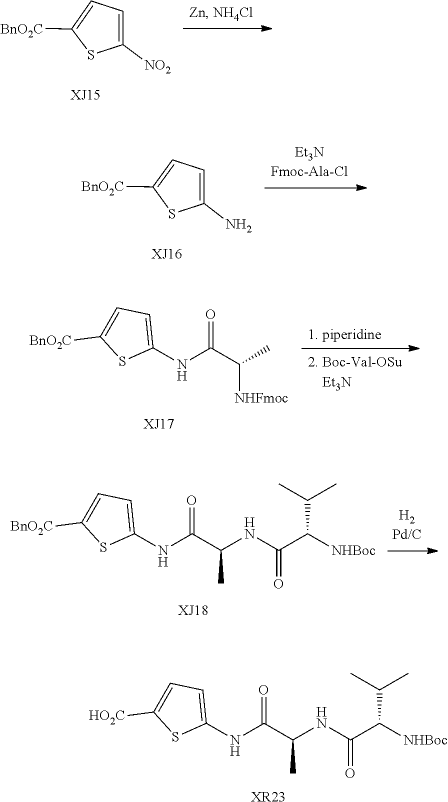

### Benzyl 5-aminothiophene-2-carboxylate (XJ16)

To a suspension of benzyl 5-nitrothiophen-2-carboxylate (XJ15) (1.55 g, 5.89 mmol, prepared as described in WO 2007/018508) in MeOH (50 mL) were added sat. aq. NH₄Cl (7 mL) and zinc powder (3.8 g) The suspension was stirred at RT for 2.5 h and was then filtered over Celite. The solid was washed with MeOH and the filtrate was concentrated. EtOAc was added and the organic solution was washed with brine, dried (Na₂SO₄), filtered, concentrated, and purified using flash chromatography (silica gel, DCM:EtOAc 1:0 to 4:1) to give XJ16 (0.90 g, 66%) as a purple solid.

¹H NMR (400 MHz, CDCl₃) ppm=7.45 (d, J=4.0 Hz, 1H), 7.40-7.29 (m, 5H), 6.03 (d, J=4.0 Hz, 1H), 5.26 (s, 2H), 1.52 (br s, 2H).

¹³C NMR (100 MHz, CDCl₃) ppm=162.5, 159.5, 136.4, 135.3, 128.5, 128.0, 117.1, 107.8, 66.1.

MS (ESI⁺) calc. for C₁₂H₁₂NO₂S⁺ [M+H]⁺ 234.05, found 234.06.

### Benzyl (S)-5-(2-((((9H-fluoren-9-yl)methoxy)carbonyl)amino)propanamido)thiophene-2-carboxylate (XJ17)

To a suspension of Fmoc-Ala-OH (1.82 g, 5.84 mmol) in DCM (18 mL) was added oxalyl chloride (1.0 mL, 11.7 mmol) and 10 drops of DMF at 0° C. The mixture was stirred at RT for 1 h and was then concentrated. The crude acid chloride was taken up in DCM (8 mL) and was subsequently added to a cooled (0° C.) solution of XJ16 (0.90 g, 3.90 mmol) in DCM (8 mL). Et₃N (1.6 mL, 11.7 mmol) was added and the mixture was stirred at RT for 3 h. The reaction mixture was concentrated and the crude product was purified by flash chromatography (silica gel, DCM:EtOAc 1:0 to 1:1) to give XJ17 (0.66 g, 32%) as a yellow foam.

MS (ESI⁺) calc. for C₃₀H₂₇N₂O₅S⁺ [M+H]⁺ 527.16, found 527.36.

### Benzyl 5-((R)-2-((R)-2-((tert-butoxycarbonyl)amino)-3-methylbutanamido)propanamido) thiophene-2-carboxylate (XJ18)

Fmoc-protected amine XJ17 (0.66 g, 1.25 mmol) was dissolved in DMF (10 mL). Piperidine (0.62 mL, 6.2 mmol) was added at RT and the mixture was stirred for 30 min. The reaction mixture was concentrated and coevaporated with toluene/DCM, to give the deprotected amine as a yellow solid. The material was redissolved in DCM (10 mL), Boc-L-Val-OSu (0.55 g, 1.75 mmol) in DCM (10 mL) and DIPEA (0.54 mL, 3.1 mmol) were added at 0° C., and the mixture was then stirred at RT for 2.5 h. After concentration, the crude was purified by flash chromatography (silica gel, DCM:EtOAc 1:0 to 4:1) to give amide XJ18 (0.48 g, 77%) as a colorless syrup.

¹H NMR (400 MHz, CDCl₃) ppm=10.76 (br s, 1H), 7.61 (d, J=4.2 Hz, 1H), 7.50 (d, J=7.2 Hz, 1H), 7.42-7.28 (m, 5H), 6.72 (d, J=3.7 Hz, 1H), 5.59 (d, J=6.7 Hz, 1H), 5.29 (s, 2H), 4.71 (q, J=7.1 Hz, 1H), 4.02 (br t, J=5.6 Hz, 1H), 2.15 (m, 1H), 1.45 (d, J=7.1 Hz, 3H), 1.42 (s, 9H), 0.96 (dd, J=17.3, 6.8 Hz, 6H).

¹³C NMR (100 MHz, CDCl₃) ppm=172.1, 169.6, 162.7, 146.0, 136.0, 131.9, 128.4, 128.0, 127.9, 123.5, 112.4, 80.0, 66.1, 60.4, 48.8, 30.6, 28.2, 19.2, 17.7, 17.5.

MS (ESI⁺) calc. for C₂₅H₃₄N₃O₆S⁺ [M+H]⁺ 504.21, found 504.31.

### 5-((R)-2-((R)-2-((tert-Butoxycarbonyl)amino)-3-methylbutanamido)propanamido) thiophene-2-carboxylic acid (XR23)

To a solution of amide XJ18 (0.48 g, 0.95 mmol) in EtOAc (10 mL) under N₂ atmosphere, was added palladium (0.3 g, 10% on activated carbon) and the mixture was stirred under hydrogen atmosphere for 2 days at RT. The reaction mixture was purged with N₂ and filtered over Celite. The residue was washed with EtOAc and the filtrate was concentrated to give XR23 (0.28 g, 72%) as a white solid.

¹H NMR (400 MHz, DMSO-d₆) ppm=12.59 (br s, 1H), 11.62 (s, 1H), 8.24 (d, J=6.6 Hz, 1H), 7.51 (d, J=4.0 Hz, 1H), 6.70 (s, 1H), 6.69-6.68 (m, 1H), 4.45-4.38 (m, 1H), 3.84 (br t, J=7.75 Hz, 1H), 1.99-1.91 (m, 1H), 1.38 (s, 9H), 1.31 (d, J=7.1 Hz, 3H), 0.87 (d, J=6.7 Hz, 3H), 0.82 (d, J=6.7 Hz, 3H).

¹³C NMR (100 MHz, CDCl₃) ppm=171.8, 170.5, 164.0, 156.0, 146.1, 131.9, 124.2, 112.3, 78.5, 59.8, 48.8, 30.9, 28.6, 19.6, 18.5, 18.1.

MS (ESI⁺) calc. for C₁₈H₂₈N₃O₆S⁺ [M+H]⁺ 414.16, found 414.29.

### Preparation of (S)-2-(4-(((2,4-diaminopteridin-6-yl)methyl)amino)benzamido)-5-(5-((S)-2-((S)-2-(6-(2,5-dioxo-2,5-dihydro-1H-pyrrol-1-yl)hexanamido)-3-methylbutanamido) propanamido)thiophene-2-carboxamido)pentanoic acid (XR16)

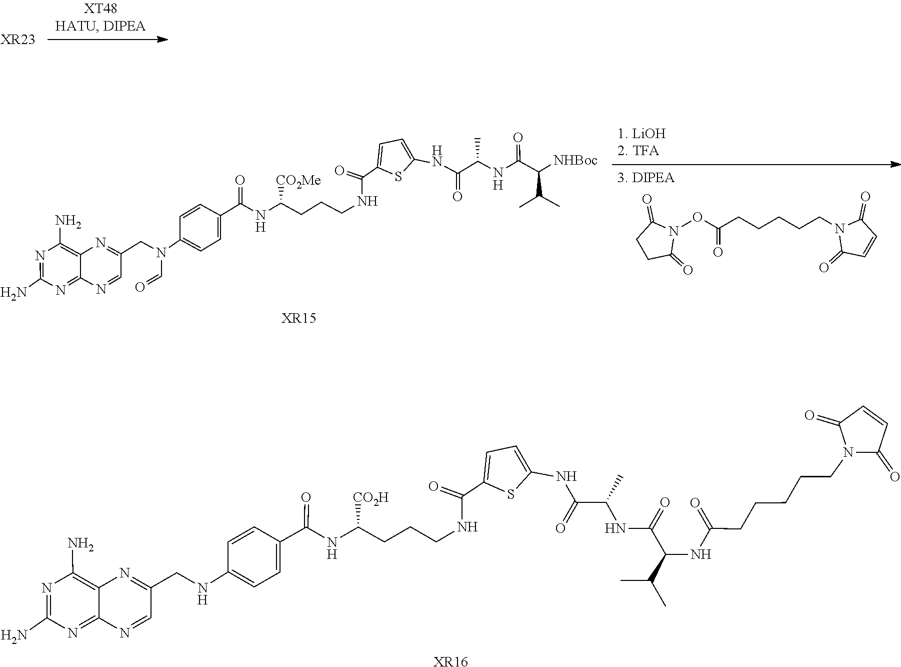

### Methyl (S)-5-(5-((S)-2-((S)-2-((tert-butoxycarbonyl)amino)-3-methylbutanamido) propanamido)thiophene-2-carboxamido)-2-(4-(N-((2,4-diaminopteridin-6-yl)methyl) formamido)benzamido) pentanoate (XR15)

Amine XT48 (50 mg, 0.1 mmol) was reacted with XR23 (41.0 mg, 0.099 mmol), HATU (45.3 mg, 0.119 mmol) and DIPEA (0.104 mL, 0.595 mmol) in DMF (1.0 mL) according to general procedure XXA. Purification of the crude product by flash chromatography (silica gel, DCM:MeOH, 1:0 to 4:1) afforded impure XR15 (105 mg) that was carried forward without any further purification.

MS (ESI⁺) calc. for C₃₉H₅₁N₁₂O₉S⁺ [M+H]⁺ 863.36, found 863.83.

### (S)-2-(4-(((2,4-Diaminopteridin-6-yl)methyl)amino)benzamido)-5-(5-((S)-2-((S)-2-(6-(2,5-dioxo-2,5-dihydro-1H-pyrrol-1-yl)hexanamido)-3-methylbutanamido)propanamido) thiophene-2-carboxamido)pentanoic acid (XR16)

A solution of amide XR15 (43 mg, 0.050 mmol) in THF/water (1:1, 1 mL) was treated with LiOH (6 mg, 0.25 mmol) at RT for 90 min. AcOH (0.03 mL, 0.5 mmol) and toluene (5 mL) were added and the mixture was concentrated. The crude was dissolved in DCM (2 mL) and TFA (1 mL) was added at 0° C. After 40 min, the mixture was concentrated, coevaporated with DCM and dried in vacuo. The deprotected intermediate was dissolved in DMF (0.5 mL), and 6-maleimidohexanoic acid N-hydroxylsuccinimide ester (18.43 mg, 0.060 mmol) was added at RT, followed by the addition of DIPEA (0.017 mL, 0.100 mmol). More DIPEA (>5 equivalents) was added to make the reaction mixture basic enough and compensate for residual acid. The mixture was stirred for 3 h at RT and was subsequently concentrated. Purification by preparative RP-HPLC (water×0.1% TFA/MeCN×0.1% TFA, gradient) afforded XR16 (11.6 mg, 26%) as a yellow solid.

¹H NMR (400 MHz, DMSO-d₆) ppm=12.95 (br s, 1H), 12.47 (m, 1H), 11.3 (s, 1H), 9.12 (br s, 1H), 8.82 (s, 1H), 8.29 (d, J=6.7 Hz, 1H), 8.26 (t, J=5.6 Hz, 1H), 8.19 (d, J=7.9 Hz, 1H), 7.80 (d, J=8.5 Hz, 1H), 7.74 (d, J=8.8 Hz, 2H), 7.49 (d, J=4.1 Hz, 1H), 7.00 (s, 2H), 6.88 (m, 1H), 6.74 (d, J=8.8 Hz, 2H), 6.65 (d, J=4.1 Hz, 1H), 4.60 (d, J=4.8 Hz, 2H), 4.36 (m, 2H), 4.15 (m, 1H), 3.21 (m, 3H), 2.23-1.89 (m, 4H), 1.89-1.68 (m, 2H), 1.65-1.42 (m, 6H), 1.31 (d, J=7.2 Hz, 3H), 1.26-1.13 (m, 4H), 0.86 (d, J=6.7 Hz, 3H), 0.82 (d, J=6.8 Hz, 3H).

MS (ESI⁺) calc. for C₄₂H₅₂N₁₃O₉S⁺ [M+H]⁺ 914.37, found 914.49.

### Preparation of methyl (R)-2-(4-(((2,5-diaminopteridin-6-yl)methyl)amino)benzamido)-5-(5-((R)-2-((R)-2-(6-(2,5-dioxo-2,5-dihydro-1H-pyrrol-1-yl)hexanamido)-3-methyl-butanamido) propanamido)thiophene-2-carboxamido)pentanoate (XJ21)

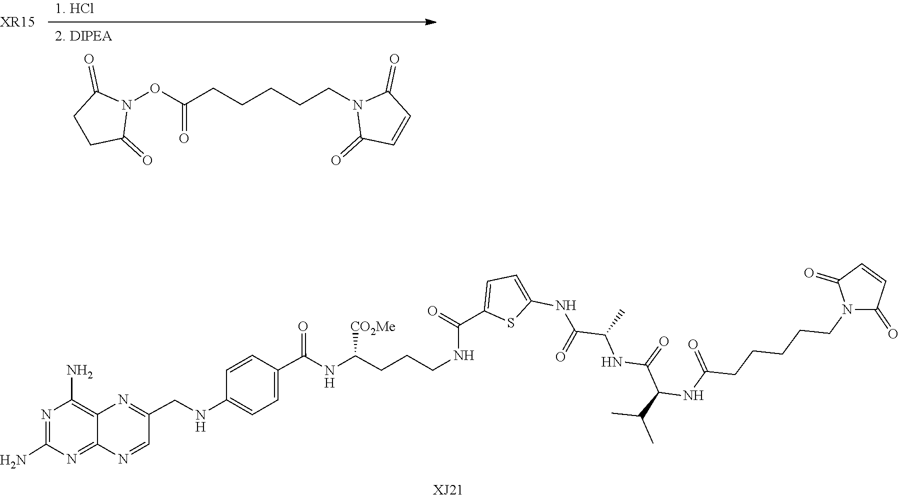

XR15 (83 mg, 0.096 mmol) was dissolved in MeOH (1.5 mL) and aq. HCl (6 N, 0.9 mL, 5.51 mmol) was added. The mixture was stirred at 50° C. for 1 h and was then cooled to RT, diluted with MeOH, coevaporated with toluene (2×), and dried in vacuo. The product was dissolved in DMF (1 mL), and 6-maleimidohexanoic acid N-hydroxylsuccinimide ester (0.029 g, 0.095 mmol) and DIPEA (0.1 mL, 0.57 mmol) were sequentially added at 0° C. The resulting mixture was stirred at RT for 2 h and was subsequently concentrated. The crude solid was purified by preparative RP-HPLC (water×0.1% TFA/MeCN×0.1% TFA, gradient 20% to 50%). Product fractions were pooled, MeCN was removed by rotary evaporation, and the aq. solution was lyophilized to yield XJ21 (25 mg, 28%) as a yellow foam.

1H NMR (400 MHz, DMSO-d₆) ppm=11.32 (s, 1H), 9.30 (bs, 1H), 9.26 (bs, 1H), 8.84 (s, 1H), 8.32 (dd, J=15.5, 7.0 Hz, 2H), 8.27-8.24 (m, 1H), 7.81 (d, J=7.8 Hz, 1H), 7.73 (d, J=8.7 Hz, 2H), 7.61 (br d, J=8.6 Hz, 1H), 7.49 (d, J=4.1 Hz, 1H), 7.0 (s, 2H), 6.91 (br s, 1H), 6.75 (d, J=8.7 Hz, 2H), 6.66 (d, J=4 Hz, 1H), 4.62 (br s, 2H), 4.43-4.34 (m, 2H), 4.15 (t, J=7.7 Hz, 1H), 3.62 (s, 3H), 3.24-3.19 (m, 2H), 2.22-2.07 (m, 2H), 1.99-1.90 (m, 1H), 1.86-1.76 (m, 2H), 1.65-1.42 (m, 7H), 1.31 (d, J=7.1 Hz, 3H), 1.22-1.14 (m, 2H), 1.04 (d, J=6.1 Hz, 2H), 0.86 (br d, J=6.7 Hz, 3H), 0.82 (br d, J=6.7 Hz, 3H).

MS (ESI⁺) calc. for C₄₃H₅₄N₁₃O₉S⁺ [M+H]⁺ 928.38, found 928.60.

### Preparation of methyl 2-ethynyl-5-nitrobenzoate (XR18)

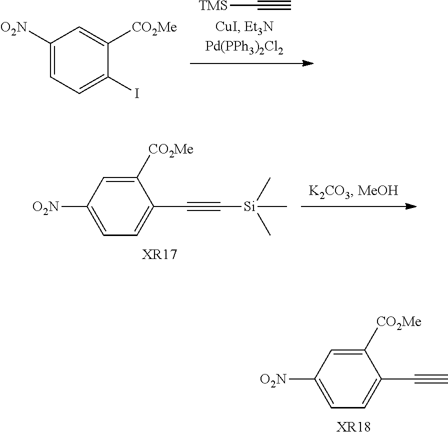

### Methyl 5-nitro-2-((trimethylsilyl)ethynyl)benzoate (XR17)

A microwave vial was charged with ethynyltrimethylsilane (0.71 mL, 5.2 mmol), methyl 2-iodo-5-nitrobenzoate (1.06 g, 3.44 mmol) and Cu(I)I (33 mg, 0.17 mmol) and DMF (15 mL), and the solution was purged with N₂. Pd(PPh₃)₂Cl₂ (48 mg, 0.069 mmol) and Et₃N (0.48 mL, 3.4 mmol) were added and the mixture was again purged with N₂. The vial was capped and heated at 80° C. for 3.5 h in a microwave. The mixture was then concentrated and the crude product was purified by flash chromatography (silica gel, heptane:EtOAc, 1:0 to 0:1) to give TMS-alkyne XR17 (620 mg, 65%).

¹H NMR (400 MHz, CDCl₃) ppm=8.77 (d, J=2.4 Hz, 1H), 8.28 (dd, J=8.6 Hz, 2.5 Hz, 1H), 7.74 (d, J=8.5 Hz, 1H), 3.98 (s, 3H), 0.31 (s, 9H).

¹³C NMR (100 MHz, CDCl₃) ppm=164.7, 146.6, 135.5, 133.6, 129.7, 125.8, 125.6, 106.9, 101.3, 52.6,−0.4.

### Methyl 2-ethynyl-5-nitrobenzoate (XR18)

Alkyne XR17 (822 mg, 2.96 mmol) and K₂CO₃ (41.0 mg, 0.296 mmol) were dissolved in MeOH (15 mL) at RT, and the mixture was stirred for 30 min. EtOAc (50 mL) was added, and the mixture was washed with sat. aq. NaHCO₃ (2×), water and brine. The organic layer was dried over MgSO₄, filtered and concentrated to give XR18 (quant. yield).

¹H NMR (400 MHz, CDCl₃) ppm=8.81 (d, J=2.4 Hz, 1H), 8.32 (dd, J=8.6 Hz, 2.5 Hz, 1H), 7.80 (d, J=8.5 Hz, 1H), 4.00 (s, 3H), 3.72 (s, 1H).

¹³C NMR (100 MHz, CDCl₃) ppm=164.3, 147.0, 136.0, 133.8, 129.0, 126.1, 125.5, 87.9, 80.4, 52.8.

### Preparation of (S)-5-azido-2-(((benzyloxy)carbonyl)amino)pentanoic acid (XR19)

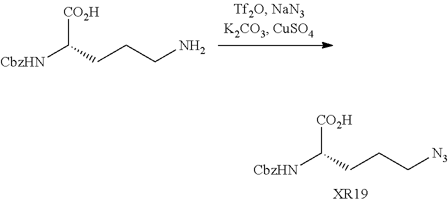

To an ice cold solution of NaN₃ (10 g, 154 mmol) in water (25 mL)/DCM (44 mL) was dropwise added triflic anhydride (5.2 mL, 30.8 mmol). The resulting mixture was stirred for 2 h at 0° C. The organic layer was separated and the aq. phase was extracted with DCM (2×20 mL). The combined organic layers were washed with aq. Na₂CO₃ (1 M), and added to a RT solution of Z-Orn-OH (4.10 g, 15.4 mmol), K₂CO₃ (3.40 g, 24.6 mmol) and CuSO₄·5H₂O (0.077 g, 0.308 mmol) in a water/MeOH (1:2, 150 mL) mixture. After stirring for 18 h at RT, the organic solvents were evaporated and the aq. slurry was diluted with water (150 mL), and acidified to pH 2 with conc. HCl. The aq. layer was extracted with EtOAc (3×), and the combined organic layers were dried over MgSO₄, filtered and concentrated in vacuo to yield crude XR19 (4.5 g) that was carried forward without further purification.

¹H NMR (400 MHz, CDCl₃) ppm=7.36 (m, 5H), 5.34 (d, J=8.3 Hz, 1H), 5.12 (s, 2H), 4.43 (m, 1H), 3.34 (m, 2H), 1.99 (m, 1H), 1.84-1.61 (m, 3H).

MS (ESI⁺) calc. for C₁₃H₁₇N₄O₄⁺ [M+H]⁺ 293.12, found 293.39.

### Preparation of (S)-5-amino-2-(1-(4-carboxy-4-(4-(((2,4-diaminopteridin-6-yl)methyl)amino) benzamido)butyl)-1H-1,2,3-triazol-4-yl)benzoic acid (XR22)

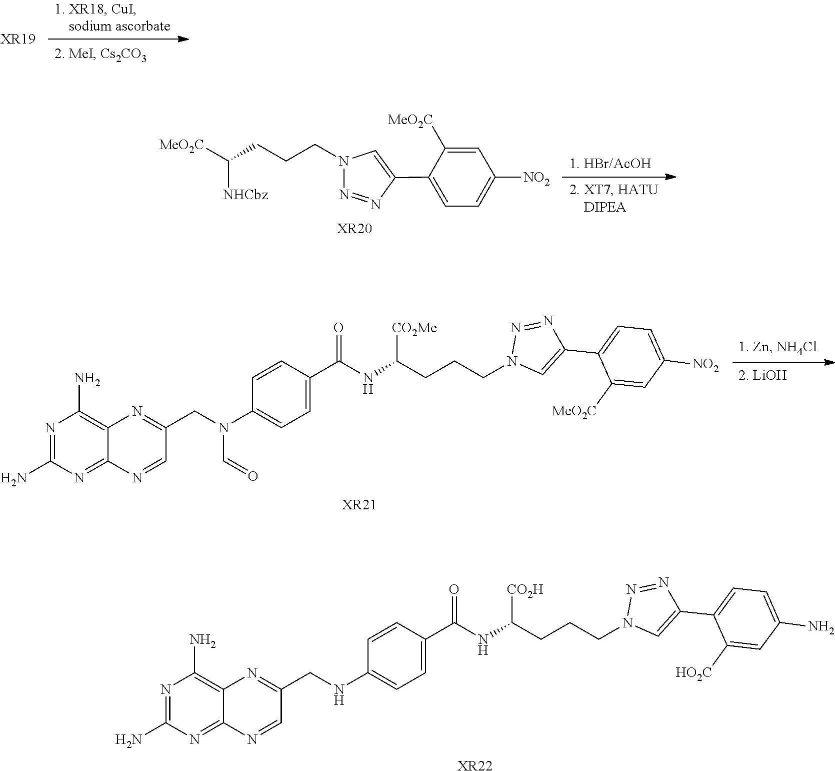

### Methyl (S)-2-(1-(4-(((benzyloxy)carbonyl)amino)-5-methoxy-5-oxopentyl)-1H-1,2,3-triazol-4-yl)-5-nitrobenzoate (XR20)

To a solution of azide XR19 (533 mg, 0.912 mmol), alkyne XR18 (187 mg, 0.912 mmol) in THF (7 mL) was added CuSO₄·5H₂O (175 mg, 0.702 mmol) in water (1.5 mL) at RT. The solution was purged with N₂ for 5 min, after which sodium ascorbate (271 mg, 1.39 mmol) in water (1.5 mL) was added. The yellow solution was stirred for 5 min at which point UPLC-MS analysis indicated full conversion. EtOAc (20 mL) and water/brine (1:1, 10 mL) were added, the layers were separated and the aq. phase was extracted with EtOAc (2×). The combined organic layers were washed with brine, dried over MgSO₄, filtered and concentrated. Purification by flash chromatography (silica gel, heptane:EtOAc, 1:0 to 0:1) afforded the triazole product. The isolated product was dissolved in DMF (10 mL), Cs₂CO₃ (163 mg, 0.500 mmol) and Mel (60 μL, 0.964 mmol) were added at RT, and the reaction was stirred 16 h. After concentration, the crude was purified by flash chromatography (silica gel, heptane:EtOAc, 1:0 to 0:1) to give ester XR20 (380 mg, 81%, 2 steps).

¹H NMR (400 MHz, CDCl₃) ppm=8.76 (d, J=2.4 Hz, 1H), 8.39 (dd, J=8.7 Hz, 2.4 Hz, 1H), 8.19 (d, J=8.7 Hz, 1H), 8.06 (s, 1H), 7.34 (m, 5H), 5.43 (d, J=7.9 Hz, 1H), 5.12 (s, 2H), 4.47 (m, 3H), 3.91 (s, 3H), 3.75 (s, 3H), 2.11-1.91 (m, 2H), 1.71 (m, 2H).

MS (ESI⁺) calc. for C₂₄H₂₆N₅O₈⁺ [M+H]⁺ 512.18, found 512.33.

### Methyl (S)-2-(1-(4-(4-(N-((2,4-diaminopteridin-6-yl)methyl)formamido)benzamido)-5-methoxy-5-oxopentyl)-1H-1,2,3-triazol-4-yl)-5-nitrobenzoate (XR21)

HBr in acetic acid (33%, 5 mL) was added to a solution of ester XR20 (300 mg, 0.587 mmol) at 0° C., and the reaction was stirred for 75 min at 0° C. The mixture was concentrated and coevaporated with toluene (2×), chloroform and ether.

MS (ESI⁺) calc. for C₁₆H₂₀N₅O₆⁺ [M+H]⁺ 378.14, found 378.19.

The crude product was reacted with XT7 (166 mg, 0.489 mmol), HATU (223 mg, 0.59 mmol) and DIPEA (0.513 mL, 2.93 mmol) in DMF (5 mL) according to general procedure XXA. Purification by flash chromatography (silica gel, DCM:MeOH, 1:0 to 4:1) afforded amide XR21 (240 mg, 70%).

MS (ESI⁺) calc. for C₃₁H₃₁N₁₂O₈⁺ [M+H]⁺ 699.24, found 699.62.

### (S)-5-Amino-2-(1-(4-carboxy-4-(4-(((2,4-diaminopteridin-6-yl)methyl)amino)benzamido) butyl)-1H-1,2,3-triazol-4-yl)benzoic acid (XR22)

To a solution of amide XR21 (280 mg, 0.423 mmol) in DMF (3 mL) was added a sat. aq. NH₄Cl (600 μL) and zinc powder (830 mg, 12.7 mmol). Once UPLC analysis indicated full conversion (˜3 h), the reaction mixture was diluted with DMF (6 mL), and filtered over Celite. The filtrate was stirred under air overnight and was subsequently concentrated, suspended in MeOH (6 mL), stirred for 30 min and filtered. The solids were collected and washed with ether, dried on air and used without further purification in the next step. The crude intermediate (109 mg) was dissolved in THF/water (1:1 2 mL). LiOH (36 mg, 0.85 mmol) was added and the mixture was stirred for 6 h. The reaction mixture was acidified using AcOH (0.100 mL, 1.7 mmol), concentrated and coevaporated with toluene (2×). A portion of the crude product was purified by preparative RP-HPLC (water×0.1% TFA/MeCN×0.1% TFA, gradient) to give XR22 (11 mg).

¹H NMR (400 MHz, DMSO-d₆) ppm=12.57 (br s, 2H), 9.32 (d, J=14.1 Hz, 2H), 8.84 (s, 1H), 8.61 (br s, 1H), 8.22 (d, J=7.8 Hz, 1H), 8.05 (s, 1H), 7.90 (br s, 1H), 7.73 (d, J=8.7 Hz, 2H), 7.35 (d, J=8.5 Hz, 1H), 6.88 (d, J=2.4 Hz, 1H), 6.74 (m, 3H), 4.62 (s, 2H), 4.38 (m, 3H), 2.00-1.71 (m, 4H).

MS (ESI⁺) calc. for C₂₈H₂₉N₁₂O₅⁺ [M+H]⁺ 613.24, found 613.37.

### Methyl (S)-2-(4-aminobenzamido)-5-((tert-butoxycarbonyl)amino)pentanoate (XT106)

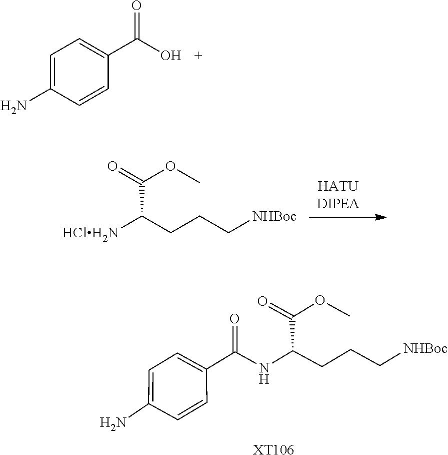

To a cooled solution of 4-aminobenzoic acid (500 mg, 3.7 mmol) and L-Orn(Boc)-OMe·HCl (1134 mg, 4.0 mmol) in DMF (15 mL) was added HATU (1664 mg, 4.4 mmol) and DIPEA (3.8 mL, 21.9 mmol), the resulting solution was stirred for 18 h at RT and was subsequently concentrated in vacuo. The residue was taken up in EtOAc, washed with sat. aq. NaHCO₃ solution and brine. The organic layer was dried over Na₂SO₄, filtered and concentrated in vacuo. The crude product was purified by flash chromatography (silica gel, DCM:EtOAc 1:0 to 0:1) to give aniline XT106 (756 mg, 57%) as a white solid.

MS (ESI⁺) calc. for C₁₈H₂₈N₃O₅⁺ [M+H]⁺ 366.2, found 366.4.

### Preparation of (S)-5-(4-amino-2-(1H-tetrazol-5-yl)benzamido)-2-(4-(((2,4-diamino-5-chloroquinazolin-6-yl)methyl)amino)benzamido)pentanoic acid (XT110)

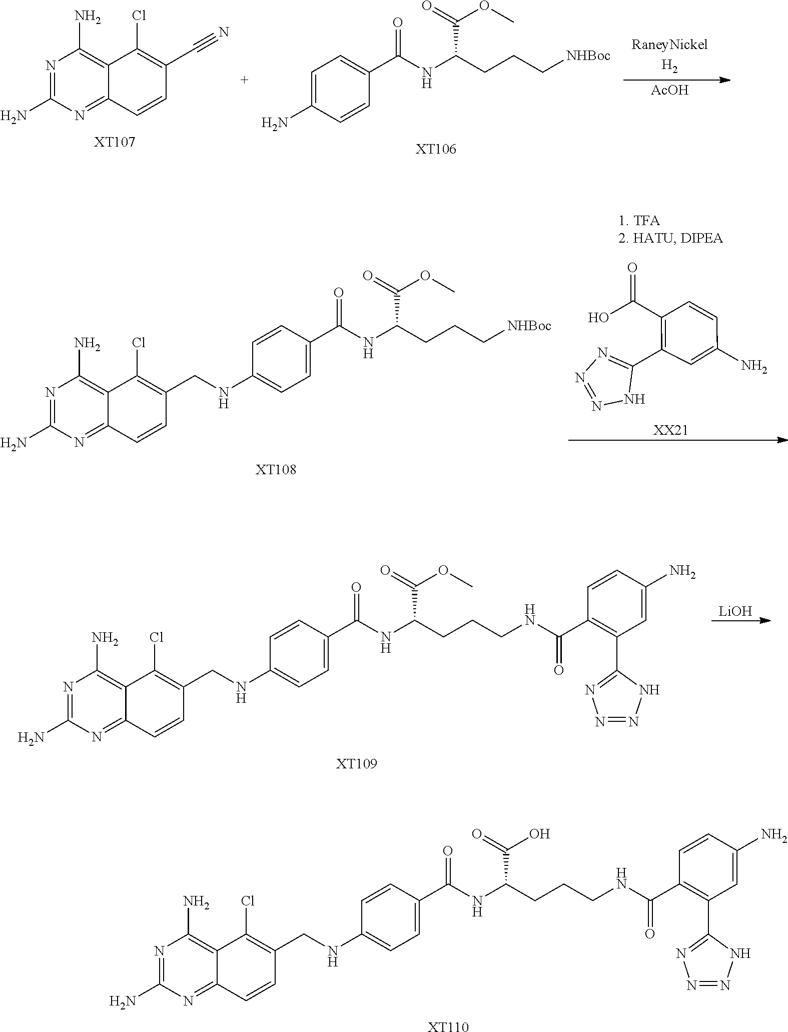

Nitrile XT107 was synthesized from commercially available 2-amino-6-chlorobenzonitrile according to the procedures described by Davoll et al (J. Chem. Soc. Sec.C Org. Chem. 1970, 8, 997-1002).

### Methyl (S)-5-((tert-butoxycarbonyl)amino)-2-(4-(((2,4-diamino-5-chloroquinazolin-6-yl) methyl)amino)benzamido)pentanoate (XT108)

To a solution of aniline XT106 (582 mg, 1.6 mmol) and nitrile XT107 (250 mg, 1.1 mmol) in AcOH (20 mL) and formic acid (5 drops) was added a slurry of Raney Nickel 50% in water (1 mL). The resulting suspension was briefly purged with hydrogen, and then stirred under hydrogen atmosphere for 4 days at RT. The reaction mixture was then filtered over Celite® and the cake was rinsed with AcOH until the eluting filtrate was colorless. Next, the filtrate was diluted with toluene and concentrated in vacuo. The crude product was purified by flash chromatography (silica gel, DCM:MeOH 1:0 to 8:2) to give carbamate XT108 (195 mg, 30%) as a yellow solid.

¹H NMR (400 MHz, DMSO-d₆) δ ppm 12.00 (br s, 1H), 8.20 (d, J=7.5 Hz, 1H), 7.75-7.52 (m, 3H), 7.48 (d, J=8.8 Hz, 1H), 7.17 (d, J=8.6 Hz, 1H), 6.84 (s, 1H), 6.77 (br t, J=5.1 Hz, 1H), 6.57 (d, J=8.6 Hz, 2H), 6.44-6.26 (m, 2H), 4.39 (br d, J=5.8 Hz, 2H), 4.37-4.29 (m, 1H), 3.61 (s, 3H), 2.96-2.88 (m, 2H), 1.81-1.65 (m, 2H), 1.51-1.37 (m, 2H), 1.36 (s, 9H).

MS (ESI⁺) calc. for C₂₇H₃₅ClN₇O₅⁺ [M+H]⁺ 572.2, found 572.4.

### Methyl (S)-5-(4-amino-2-(1H-tetrazol-5-yl)benzamido)-2-(4-(((2,4-diamino-5-chloroquinazolin-6-yl)methyl)amino)benzamido)pentanoate (XT109)

Following general procedure XXD, carbamate XT108 (30 mg, 0.05 mmol) was reacted with a 4-amino-2-(2H-tetrazol-5-yl)benzoic acid solution XX21 (0.39 M, 136 μL, 0.05 mmol) in DMF to afford methyl ester XT109 (20 mg, 58%) as a brown solid.

MS (ESI⁺) calc. for C₃₀H₃₂ClN₁₂O₄⁺ [M+H]⁺ 659.2, found 659.4.

### (S)-5-(4-Amino-2-(1H-tetrazol-5-yl)benzamido)-2-(4-(((2,4-diamino-5-chloroquinazolin-6-yl)methyl)amino)benzamido)pentanoic acid (XT110)

Saponification of methyl ester XT109 (20 mg, 0.03 mmol) was performed according to general procedure XXE. The crude solid was purified by preparative RP-HPLC (water×0.1% TFA/MeCN, gradient 5% to 35%). Combined product fractions were concentrated to remove MeCN followed by lyophilization to yield compound XT110 (9 mg, 46%) as a yellow solid.

¹H NMR (400 MHz, DMSO-d₆) δ ppm 12.87-12.15 (m, 2H), 9.27 (s, 1H), 8.63 (s, 1H), 8.14 (br s, 1H), 8.10 (br d, J=7.8 Hz, 1H), 8.02-7.73 (m, 2H), 7.70 (d, J=8.8 Hz, 1H), 7.66 (d, J=8.6 Hz, 2H), 7.43 (s, 1H), 7.39 (d, J=8.6 Hz, 1H), 7.02-6.86 (m, 1H), 6.70 (br d, J=6.5 Hz, 2H), 6.56 (d, J=8.8 Hz, 2H), 6.19-5.09 (m, 2H), 4.46 (s, 2H), 4.35-4.25 (m, 1H), 3.16-3.06 (m, 2H), 1.88-1.62 (m, 2H), 1.62-1.40 (m, 2H).

MS (ESI⁺) calc. for C₂₉H₃₀ClN₁₂O₄⁺ [M+H]⁺ 645.2, found 645.6.

### Preparation of (S)-2-(4-(((2,4-diamino-5-chloroquinazolin-6-yl)methyl)amino) benzamido)-5-(4-((S)-2-((S)-2-(6-(2,5-dioxo-2,5-dihydro-1H-pyrrol-1-yl)hexanamido)-3-methylbutanamido)propanamido)-2-(1H-tetrazol-5-yl)benzamido)pentanoic acid (XT114)

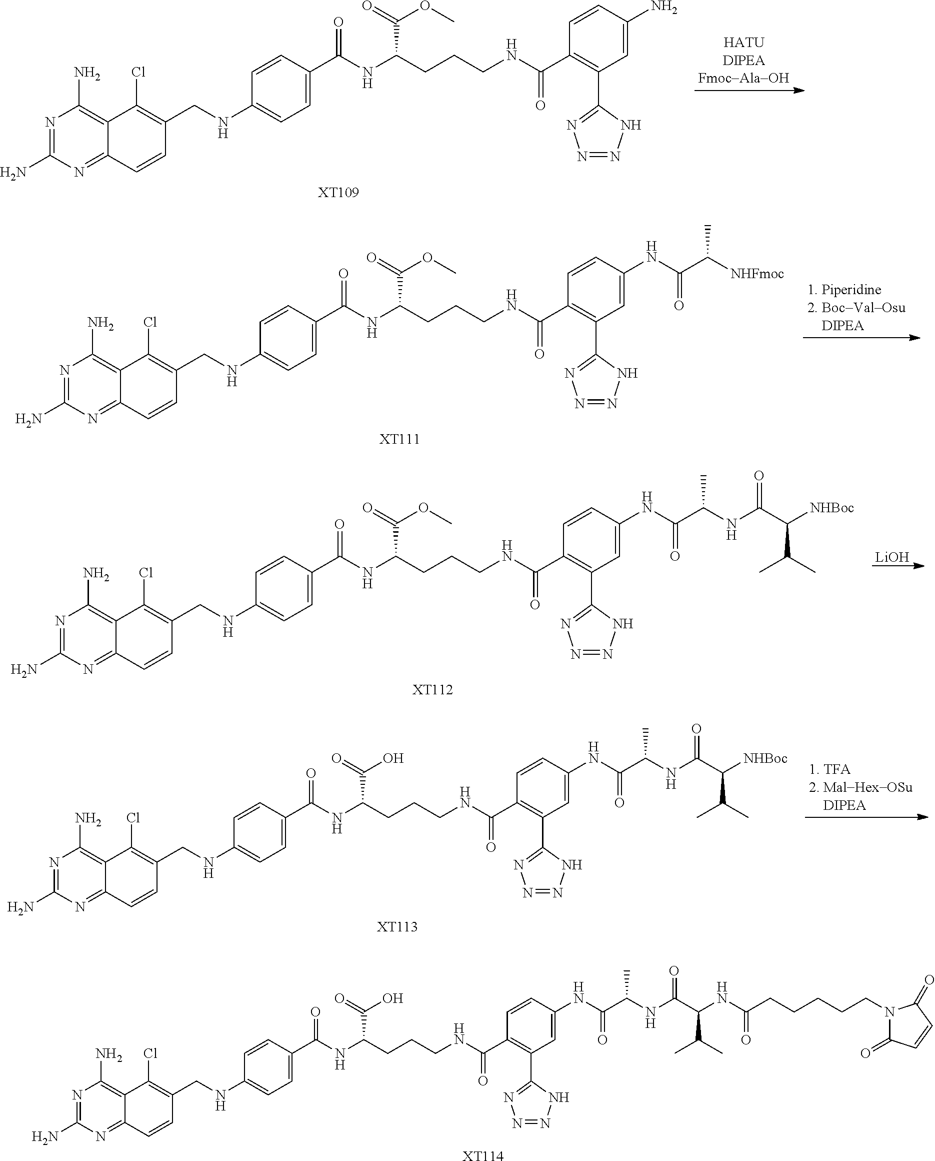

### Methyl (S)-5-(4-((S)-2-((((9H-fluoren-9-yl)methoxy)carbonyl)amino)propanamido)-2-(1H-tetrazol-5yl)benzamido)-2-(4-(((2,4-diamino-5-chloroquinazolin-6-yl)methyl)amino) benzamido)pentanoate (XT111)

Using general procedure XXA, aniline XT109 (160 mg, 0.24 mmol) was reacted with Fmoc-L-Ala-OH (83 mg, 0.27 mmol). Purification by trituration with MeCN afforded carbamate XT111 (quant) as a brown solid.

MS (ESI⁺) calc. for C₄₈H₄₇ClN₁₃O₇⁺ [M+H]⁺ 952.3, found 952.7.

### Methyl (S)-5-(4-((S)-2-((S)-2-((tert-butoxycarbonyl)amino)-3-methylbutanamido) propanamido)-2-(H-tetrazol-5-yl)benzamido)-2-(4-(((2,4-diamino-5-chloroquinazolin-6-yl)methyl)amino)benzamido)pentanoate (XT112)

To a solution of carbamate XT111 (230 mg, 0.24 mmol) in DMF (4 mL) was added piperidine (475 μL, 4.80 mmol) and the resulting solution was stirred for 10 min at RT. The reaction mixture was concentrated in vacuo and the residue was coevaporated with toluene. The obtained solid was suspended in MeCN (8 mL), stirred for 30 min and subsequently filtered and the residue taken up in DMF (2 mL). The solution was cooled and Boc-L-Val-OSu (75 mg, 0.24 mmol) and DIPEA (84 μL, 0.48 mmol) were added. The resulting mixture was stirred for 3 h and subsequently concentrated in vacuo. The crude product was suspended in MeCN (6 mL) and stirred for 15 min followed by filtration. The residue was collected to afford methyl ester XT112 (quant) as a brown solid.

MS (ESI⁺) calc. for C₄₃H₅₄ClN₁₄O₈⁺ [M+H]⁺ 929.4, found 929.8.

### (S)-5-(4-((S)-2-((S)-2-((tert-Butoxycarbonyl)amino)-3-methylbutanamido) propanamido)-2-(1H-tetrazol-5-yl)benzamido)-2-(4-(((2,4-diamino-5-chloroquinazolin-6-yl)methyl)amino) benzamido)pentanoic acid (XT113)

Saponification of methyl ester XT112 (186 mg, 0.2 mmol) was performed according to general procedure XXE. No further purification was performed.

MS (ESI⁺) calc. for C₄₂H₅₂ClN₁₄O₈⁺ [M+H]⁺ 915.4, found 915.8.

### (S)-2-(4-(((2,4-Diamino-5-chloroquinazolin-6-yl)methyl)amino)benzamido)-5-(4-((S)-2-((S)-2-(6-(2,5-dioxo-2,5-dihydro-1H-pyrrol-1-yl)hexanamido)-3-methylbutanamido) propanamido)-2-(1H-tetrazol-5-yl)benzamido)pentanoic acid (XT114)

A suspension of XT113 (146 mg, 0.16 mmol) in DCM (3 mL) was cooled to 0° C., TFA (3 mL) was added and the resulting solution was stirred at 0° C. for 1 h. The reaction mixture was then diluted with DCM and concentrated in vacuo and coevaporated twice with DCM. The residue was taken up in DMF (3 mL), cooled on an ice bath and 6-maleimidohexanoic acid N-hydroxylsuccinimide ester (49 mg, 0.16 mmol) and DIPEA (279 μL, 1.60 mmol) were added. The resulting mixture was stirred for 4 h at RT and subsequently concentrated in vacuo. The crude product was purified by preparative RP-HPLC (water×0.1% TFA/MeCN, gradient 20% to 30%). The combined product fractions concentrated in vacuo to remove MeCN. The aqueous solution was lyophilized to yield compound XT114 (24 mg, 15%) as a yellow solid.

¹H NMR (400 MHz, DMSO-d₆) δ ppm 12.67-12.26 (m, 2H), 10.27 (s, 1H), 9.26 (br s, 1H), 8.62 (br s, 1H), 8.46-8.36 (m, 1H), 8.22 (d, J=6.8 Hz, 1H), 8.10 (d, J=7.8 Hz, 1H), 7.99-7.57 (br s, 2H), 7.93 (br s, 1H), 7.79 (d, J=8.5 Hz, 2H), 7.72-7.60 (m, 4H), 7.39 (d, J=8.6 Hz, 1H), 6.99 (s, 2H), 6.94 (t, J=5.8 Hz, 1H), 6.56 (d, J=8.8 Hz, 2H), 6.64-6.43 (m, 2H), 4.46 (d, J=5.0 Hz, 2H), 4.40-4.28 (m, 2H), 4.16 (dd, J=7.1, 8.3 Hz, 1H), 3.20-3.08 (m, 2H), 2.23-2.05 (m, 2H), 2.01-1.89 (m, 1H), 1.87-1.67 (m, 2H), 1.65-1.39 (m, 6H), 1.32 (d, J 5=7.1 Hz, 3H), 1.22-1.12 (m, 2H), 0.87 (d, J=6.7, 3H), 0.82 (d, J=6.7, 3H).

MS (ESI⁺) calc. for C₄₇H₅₅ClN₁₅O₉⁺ [M+H]⁺ 1008.4, found 1009.0.

### Preparation of (S)-5-(4-amino-2-(1H-tetrazol-5-yl)benzamido)-2-(4-(2-(2,4-diamino-5-chloroquinazolin-6-yl)ethyl)benzamido)pentanoic acid (XT123)

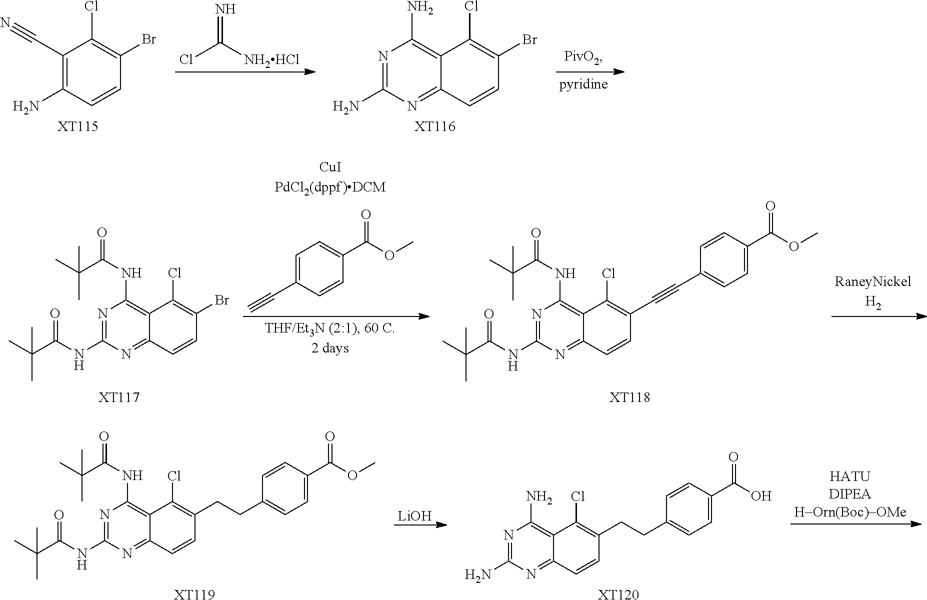

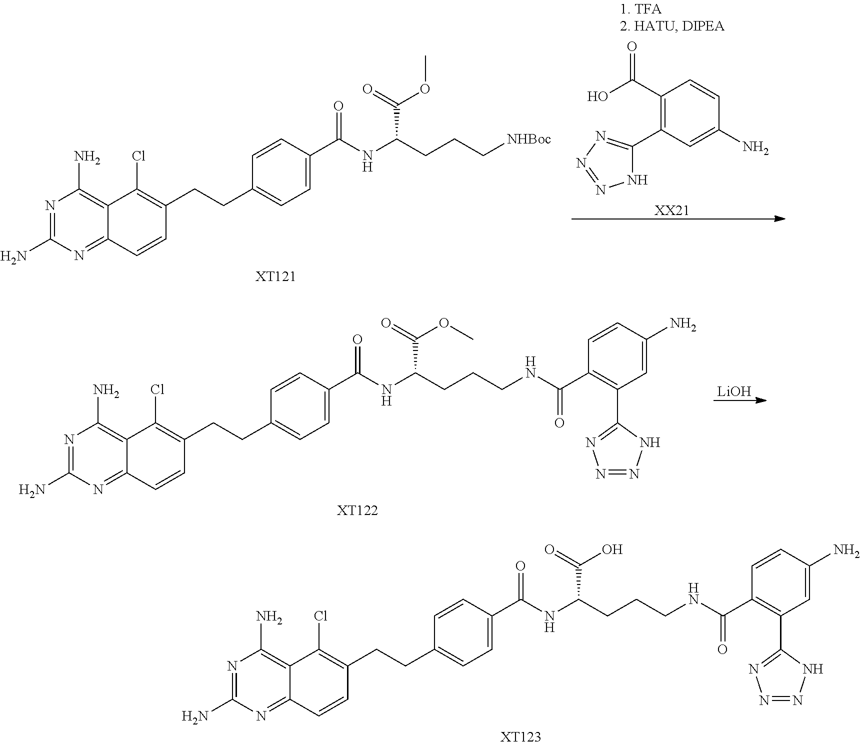

### 6-Amino-3-bromo-2-chlorobenzonitrile (XT115)

Compound XT115 was synthesized from 2-amino-6-chlorobenzonitrile according to the procedure described in U.S. Pat. No. 5,534,518.

### 6-Bromo-5-chloroquinazoline-2,4-diamine (XT116)

A suspension of chloroformamidine hydrochloride (2.5 g, 21.6 mmol) and nitril XT115 (5.0 g, 21.6 mmol) in bis(2-methoxyethyl)ether (60 mL) was heated to 165° C. under a N₂ atmosphere for 3 h. After cooling to RT, the crude mixture was diluted with ether (150 mL). The obtained suspension was filtered and the residue taken up in water (50 mL). The resulting suspension was filtered and the residue was dried on air overnight to afford chloride XT116 (1.7 g, 29%) as a light brown solid.

MS (ESI⁺) calc. for C₈H₇BrClN₄⁺ [M+H]⁺ 273.0, found 273.0.

### N,N′-(6-Bromo-5-chloroquinazoline-2,4-diyl)bis(2,2-dimethylpropanamide) (XT117)

To a suspension of chloride XT116 (1.7 g, 6.2 mmol) in pyridine (20 mL) was added pivalic anhydride (2.7 mL, 13.1 mmol), the resulting mixture was heated to 125° C. After 18 h, more pivalic anhydride (1 eq) was added and the reaction was continued at 125° C. for 6 h. After cooling to RT the crude mixture was filtered and the residue washed with EtOAc. The combined filtrates were concentrated in vacuo and the residue taken up in EtOAc (100 mL). The obtained solution was washed with aq. 1 M HCl (100 mL), the aqueous layer was extracted with EtOAc (100 mL) and the combined organic extracts were washed with brine. After drying over MgSO₄, the organic layer was filtered and concentrated in vacuo to obtain the crude product and was purified by flash chromatography (silica gel, DCM:EtOAc 1:0 to 3:1) to afford bromide XT117 (1.5 g, 55%) as a light yellow solid.

¹H NMR (400 MHz, DMSO-d₆) δ ppm 15.33-15.15 (m, 0.35H), 11.14 (br s, 0.35H), 10.58 (s, 0.65H), 10.33 (s, 0.65H), 8.17 (d, J=9.0 Hz, 0.65H), 8.07 (d, J=8.9 Hz, 0.35H), 7.72 (d, J=9.1 Hz, 0.65H), 7.34 (d, J=8.8 Hz, 0.35H), 1.35-1.17 (m, 18H) (rotamers are observed).

MS (ESI⁺) calc. for C₁₈H₂₃BrClN₄O₂⁺ [M+H]⁺ 441.1, found 441.3.

### Methyl 4-((5-chloro-2,4-dipivalamidoquinazolin-6-yl)ethynyl)benzoate (XT118)

A nitrogen purged solution of bromide XT117 (850 mg, 1.92 mmol) and methyl 4-ethynylbenzoate (616 mg, 3.85 mmol) in THF (10 mL) and Et₃N (5 mL) was added to a nitrogen purged reaction vessel containing PdCl₂(dppf).DCM (157 mg, 0.19 mmol) and copper(I) iodide (73 mg, 0.39 mmol). The resulting mixture was stirred at 60° C. for 48 h, concentrated in vacuo and the obtained crude product was purified by flash chromatography (silica gel, heptane:EtOAc 1:0 to 6:4) to afford alkyne XT118 (750 mg, 75%) as a slightly orange solid.

¹H NMR (400 MHz, DMSO-d₆) δ ppm 15.23 (s, 0.45H), 11.25-11.16 (m, 0.45H), 10.60 (s, 0.55H), 10.36 (s, 0.55H), 8.03-7.99 (m, 2.55H), 7.99-7.94 (m, 0.45H), 7.83-7.72 (m, 2.55H), 7.46-7.41 (m, 0.45H), 3.89 (s, 3H), 1.31-1.21 (m, 18H) (rotamers are observed).

MS (ESI⁺) calc. for C₂₈H₃₀ClN₄O₄⁺ [M+H]⁺ 521.2, found 521.4.

### Methyl 4-(2-(5-chloro-2,4-dipivalamidoquinazolin-6-yl)ethyl)benzoate (XT119)

To a solution of alkyne XT118 (750 mg, 1.44 mmol) in THF (30 mL) was added a slurry of Raney Nickel 50% in water (3 mL). The resulting suspension was put under a H₂ atmosphere and stirred for 7 days. Then, the reaction mixture was filtered over Celite®, the cake was rinsed with THF until a colorless filtrate was observed. The filtrate was concentrated in vacuo and purified by flash chromatography (silica gel, DCM:EtOAc 1:0 to 6:4) to afford methyl ester XT119 (480 mg, 64%) as a light brown solid.

¹H NMR (400 MHz, DMSO-d₆) δ=15.24 (s, 0.4H), 11.10-10.97 (m, 0.4H), 10.46 (br s, 0.6H), 10.22 (s, 0.6H), 7.93-7.84 (m, 2H), 7.84-7.76 (m, 1H), 7.73-7.65 (m, 1H), 7.44-7.33 (m, 2H), 3.84 (s, 3H), 3.24-3.11 (m, 2H), 3.06-2.94 (m, 2H), 1.32-1.21 (m, 18H) (rotamers are observed).

MS (ESI⁺) calc. for C₂₈H₃₄ClN₄O₄⁺ [M+H]⁺ 525.2, found 525.4.

### 4-(2-(2,4-Diamino-5-chloroquinazolin-6-yl)ethyl)benzoic acid (XT120)

To a solution of methyl ester XT119 (400 mg, 0.76 mmol) in THF (15 mL)/MeOH (0.5 mL) was added a solution of LiOH (182 mg, 7.62 mmol) in water (7.5 mL). The resulting clear solution was stirred for 48 h at RT. The reaction mixture was then cooled with an ice bath and AcOH (1.1 mL, 19.05 mmol) was added. The resulting suspension was stirred for 15 min and was subsequently filtered. The residue was washed with MeCN (5 mL) and ether (5 mL) to afford carboxylic acid XT120 (180 mg, 69%) as a light yellow solid.

¹H NMR (400 MHz, DMSO-d₆) δ=7.81 (br s, 2H), 7.41-7.29 (m, 3H), 7.19 (br s, 2H), 7.09 (br d, J=8.5 Hz, 1H), 6.07 (br s, 2H), 3.04-2.94 (m, 3H), 2.88-2.76 (m, 2H).

MS (ESI⁺) calc. for C₁₇H₁₆ClN₄O₂⁺ [M+H]⁺ 343.3, found 343.2.

### Methyl (S)-5-((tert-butoxycarbonyl)amino)-2-(4-(2-(2,4-diamino-5-chloroquinazolin-6-yl)ethyl)benzamido)pentanoate (XT121)

To a cooled suspension of carboxylic acid XT120 (49 mg, 0.14 mmol) and L-Orn(Boc)-OMe·HCl (45 mg, 0.16 mmol) in DMA (2 mL) was added HATU (65 mg, 0.17 mmol) and DIPEA (250 μL, 1.43 mmol). The resulting mixture was stirred for 120 min at RT and concentrated in vacuo. The crude product was purified by flash chromatography (silica gel, DCM:MeOH 1:0 to 9:1) to afford carbamate XT121 (80 mg, 98%) as a brown solid.

¹H NMR (400 MHz, DMSO-d₆) δ ppm 8.63 (d, J=7.4 Hz, 1H), 8.02-7.72 (br s, 2H), 7.81 (d, J=8.3 Hz, 2H), 7.48 (d, J=8.6 Hz, 1H), 7.32 (d, J=8.3 Hz, 2H), 7.18 (d, J=8.6 Hz, 1H), 6.79 (br t, J=5.2 Hz, 1H), 6.62 (br s, 2H), 4.47-4.33 (m, 1H), 3.64 (s, 3H), 3.08-2.99 (m, 2H), 2.98-2.88 (m, 4H), 1.86-1.68 (m, 2H), 1.56-1.40 (m, 2H), 1.37 (s, 9H).

MS (ESI⁺) calc. for C₂₈H₃₆ClN₆O₅⁺ [M+H]⁺ 571.2, found 571.4.

### Methyl (S)-5-(4-amino-2-(1H-tetrazol-5-yl)benzamido)-2-(4-(2-(2,4-diamino-5-chloroquinazolin-6-yl)ethyl)benzamido)pentanoate (XT122)

According to general procedure XXD, carbamate XT121 (100 mg, 0.18 mmol) was reacted with a solution of XX21 (0.39 M, 455 μL, 0.18 mmol) in DMF to afford methyl ester XT122 (56 mg, 49%) as a light brown solid.

MS (ESI⁺) calc. for C₃₁H₃₃ClN₁₁O₄⁺ [M+H]⁺ 658.2, found 658.4.

### (S)-5-(4-amino-2-(1H-tetrazol-5-yl)benzamido)-2-(4-(2-(2,4-diamino-5-chloroquinazolin-6-yl)ethyl)benzamido)pentanoic acid (XT123)

Saponification of methyl ester XT122 (56 mg, 0.09 mmol) was carried out according to general procedure XXE. The crude product was purified by preparative RP-HPLC (water×0.10% TFA/MeCN, gradient 10% to 40%). The combined product fractions were concentrated in vacuo to remove MeCN. The aqueous solution was lyophilized to yield compound XT123 (25 mg, 46%) as a white solid.

¹H NMR (400 MHz, DMSO-d₆) δ ppm 12.81-12.24 (m, 2H), 9.25 (s, 1H), 8.62 (s, 1H), 8.54 (d, J=7.6 Hz, 1H), 8.18 (br s, 1H), 8.00-7.85 (br s, 2H), 7.82 (d, J=8.1 Hz, 2H), 7.70 (d, J=8.6 Hz, 1H), 7.50-7.41 (m, 1H), 7.38-7.27 (m, 3H), 6.76-6.65 (m, 2H), 5.85 (br s, 2H), 4.42-4.29 (m, 1H), 3.16-3.06 (m, 4H), 3.00-2.88 (m, 2H), 1.90-1.68 (m, 2H), 1.64-1.42 (m, 2H).

MS (ESI⁺) calc. for C₃₀H₃₁ClN₁₁O₄⁺ [M+H]⁺ 644.2, found 644.4.

### Preparation of (S)-5-(4-amino-2-(2H-tetrazol-5-yl)benzamido)-2-(5-(((2,4-diaminopteridin-6-yl)methyl)(methyl) amino)thiophene-2-carboxamido)pentanoic acid (XC5)

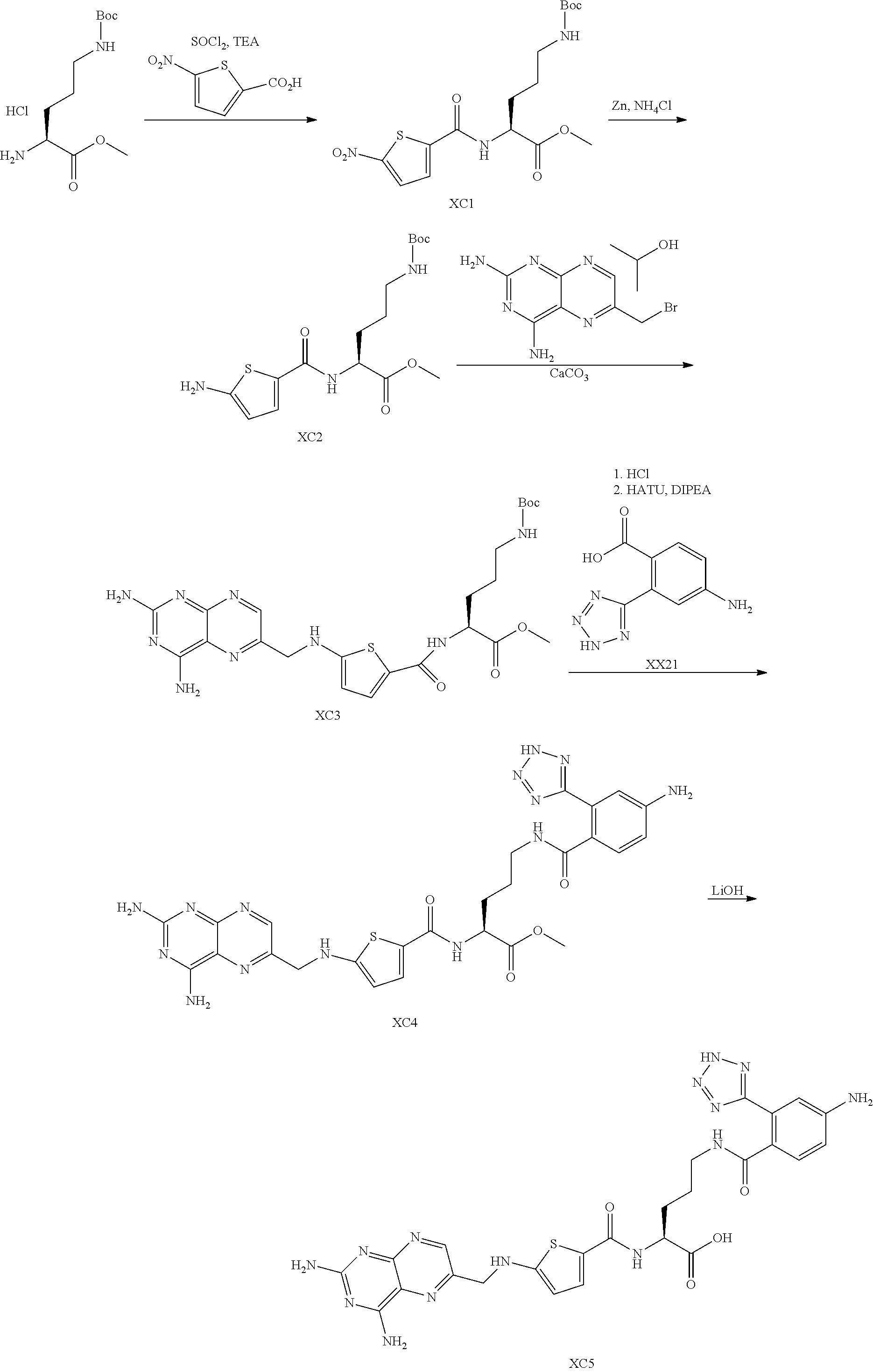

### Methyl (S)-5-((tert-butoxycarbonyl)amino)-2-(5-nitrothiophene-2-carboxamido) pentanoate (XC1)

5-Nitrothiophene-2-carboxylic acid (4.07 g, 23.52 mmol) was added slowly to SOCl₂ (18 mL, 246 mmol) and the mixture was refluxed for 30 min and evaporated in vacuo to obtain the corresponding acid chloride. This was taken up immediately in DCM (28.5 mL) and added to a mixture of L-Orn(Boc)-OMe·HCl (5 g, 17.68 mmol) in DCM (57 mL). The resulting mixture was treated immediately with triethylamine (TEA; 4.12 mL, 29.5 mmol) resulting in a moderate exotherm. After 1 h more TEA (1.2 mL) was added and the mixture was stirred for 1.5 h. The solvent was evaporated and the product was partitioned between EtOAc (150 mL) and water (100 mL). After separation, the organic layer was washed with 0.5 N HCl, water, 5% NaHCO₃ solution, brine and dried over Na₂SO₄, followed by filtration and concentration in vacuo. The crude product was purified by flash chromatography (silica gel, heptane:EtOAc 9:1 to 0:1) to afford nitro compound XC1 (5.3 g, 74%) as a foam.

¹H NMR (400 MHz, CDCl₃) δ ppm 7.99 (br s, 1H), 7.86 (d, J=4.3 Hz, 1H), 7.73-7.61 (m, 1H), 4.79-4.61 (m, 2H), 3.78 (s, 3H), 3.33-3.21 (m, 1H), 3.20-3.08 (m, 1H), 2.05-1.95 (m, 1H), 1.90-1.80 (m, 1H), 1.68-1.56 (m, 2H), 1.46 (s, 9H).

MS (ESI⁺) calc. for C₁₆H₂₃N₃NaO₇S [M+Na] 424.1, found 424.3.

### Methyl (S)-2-(5-aminothiophene-2-carboxamido)-5-((tert-butoxycarbonyl)amino) pentanoate (XC2)

To a solution of nitro compound XC1 (0.5 g, 1.246 mmol) in MeOH (9 mL) was added a saturated aqueous NH₄Cl solution (6.2 mL) and water (2 mL) followed by zinc (814 mg, 12.5 mmol). The reaction was stirred for 30 min at RT. The mixture was filtered through Celite®, rinsed with MeOH and concentrated in vacuo. The residue was partitioned between EtOAc and water. After separation, the aqueous phase was back extracted with EtOAc. The combined organic layers were washed with water, brine and dried over Na₂SO₄, filtered off and concentrated in vacuo. The crude product was purified by flash chromatography (silica gel, heptane:EtOAc 3:2 to 0:1) to afford amine XC2 (266 mg, 57%) as a pink foam.

¹H NMR (400 MHz, CDCl₃) δ ppm 7.24-7.19 (m, 1H), 6.51 (br s, 1H), 6.08 (d, J=4.0 Hz, 1H), 4.78-4.70 (m, 1H), 4.61 (br s, 1H), 4.18 (s, 2H), 3.76 (s, 3H), 3.23-3.09 (m, 2H), 2.01-1.90 (m, 1H), 1.81-1.70 (m, 1H), 1.62-1.51 (m, 2H), 1.44 (s, 9H).

MS (ESI⁺) calc. for C₁₆H₂₆N₃O₅S⁺ [M+H]⁺ 372.1, found 372.3.

### Methyl (S)-5-((tert-butoxycarbonyl)amino)-2-(5-(((2,4-diaminopteridin-6-yl)methyl)amino) thiophene-2-carboxamido)pentanoate (XC3)

A solution of 6-(bromomethyl)pteridine-2,4-diamine/2-propanol (1/1) (150 mg, 0.476 mmol, WO 98/024789) in dry DMA (3 mL) was put under a N₂ atmosphere and amine XC2 (177 mg, 0.476 mmol) and CaCO₃ (105 mg, 1.047 mmol) were added. The flask was wrapped in tinfoil and stirred at RT for 4 days. Next, the mixture was filtered over Celite®, rinsed with DMA and concentrated in vacuo. The crude product was purified by flash chromatography (silica gel, DCM:MeOH 1:0 to 8:2) to afford methyl ester XC3 (190 mg, 73%) as a brown solid.

¹H NMR (400 MHz, CD₃OD) δ ppm 8.79 (s, 1H), 7.50 (d, J=4.3 Hz, 1H), 6.07 (d, J=4.3 Hz, 1H), 4.61 (s, 2H), 4.51 (br dd, J=9.1, 4.9 Hz, 1H), 3.74 (s, 3H), 3.12-3.04 (m, 2H), 2.00-1.88 (m, 1H), 1.84-1.71 (m, 1H), 1.67-1.50 (m, 2H), 1.44 (s, 9H).

MS (ESI⁺) calc. for C₂₃H₃₂N₉O₅S⁺ [M+H]⁺ 546.2, found 546.5.

### Methyl (S)-5-(4-amino-2-(2H-tetrazol-5-yl)benzamido)-2-(5-(((2,4-diaminopteridin-6-yl) methyl)amino)thiophene-2-carboxamido)pentanoate (XC4)

To a stirred suspension of methyl ester XC3 (52 mg, 0.095 mmol) in dioxane (2 mL) was added slowly 4N HCl in dioxane (2 mL, 8.00 mmol) and the resulting mixture was stirred for 2.5 h at RT. The mixture was filtered and the residue rinsed with Et₂O, dried under high vacuum, affording a brown powder. To a cooled (0° C.) mixture of the HCl salt and a solution of XX21 (0.385 M, 0.248 mL, 0.095 mmol) in DMF were added HATU (43.5 mg, 0.114 mmol) and DIPEA (0.100 mL, 0.572 mmol), and the resulting mixture was stirred for 15 min. at 4° C. Then, concentrated in vacuo, followed by addition of MeCN (8 mL) and stirred for 0.5 h. The solids were filtered off, rinsed with MeCN and dried under high vacuum to afford amine XC4 in a quantitative yield.

MS (ESI⁺) calc. for C₂₆H₂₉N₁₄O₄S⁺ [M+H]⁺ 633.2, found 633.5.

### (S)-5-(4-amino-2-(2H-tetrazol-5-yl)benzamido)-2-(5-(((2,4-diaminopteridin-6-yl)methyl) (methyl)amino)thiophene-2-carboxamido)pentanoic acid (XC5)

Saponification of methyl ester XC4 (35 mg, 0.06 mmol) was carried out according to general procedure XXE. The crude solid was purified by preparative RP-HPLC (water×0.10% TFA/MeCN, gradient 5% to 35%). The combined product fractions were concentrated in vacuo to remove MeCN. The aqueous solution was lyophilized to yield compound XC5 (2.6 mg, 7%) as a yellow solid.

MS (ESI⁺) calc. for C₂₅H₂₇N₁₄O₄S⁺ [M+H]⁺ 619.2, found 619.5.

### Preparation of (S)-5-(4-amino-2-(2H-tetrazol-5-yl)benzamido)-2-(5-(((2,4-diaminopteridin-6-yl)methyl)(methyl)amino)thiophene-2-carboxamido)pentanoic acid (XC9)

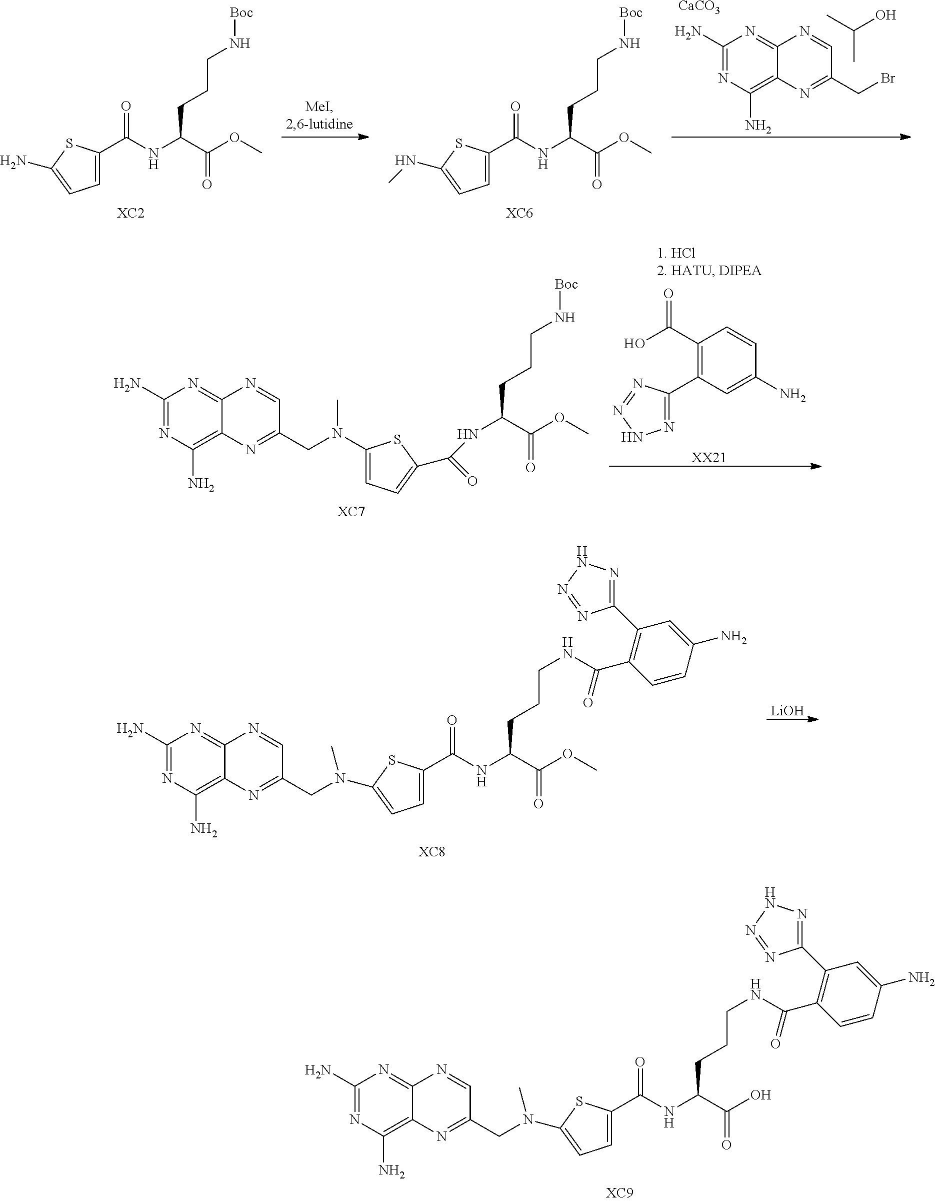

### Methyl (S)-5-((tert-butoxycarbonyl)amino)-2-(5-(methylamino)thiophene-2-carboxamido) pentanoate (XC6)

To a solution of amine XC2 (250 mg, 0.673 mmol) in DMF (5 mL) were added 2,6-lutidine (0.048 mL, 0.413 mmol) and Mel (0.044 mL, 0.700 mmol). The mixture was stirred at RT for 7 days. The mixture was quenched with brine:water (1:1, 8 mL) and extracted with EtOAc (3×10 mL). The combined organic layers were washed with water twice, brine and dried over Na₂SO₄, filtered and concentrated in vacuo. The crude product was purified by flash chromatography (silica gel, DCM:MeOH 1:0 to 9:1) to afford the methylated amine XC6 (97 mg, 32%) as a brown oil.

¹H NMR (400 MHz, CDCl₃) δ ppm 7.28 (d, J=4.1 Hz, 1H), 6.56-6.40 (m, 1H), 5.90 (d, J=4.1 Hz, 1H), 4.75 (td, J=7.6, 5.3 Hz, 1H), 4.66 (br s, 1H), 4.50-4.43 (m, 1H), 3.76 (s, 3H), 3.19-3.11 (m, 2H), 2.91 (d, J=5.3 Hz, 3H), 2.00-1.88 (m, 1H), 1.80-1.73 (m, 1H), 1.62-1.53 (m, 2H), 1.43 (s, 9H).

MS (ESI⁺) calc. for C₁₇H₂₈N₃O₅S⁺ [M+H]⁺ 386.2, found 386.34.

### Methyl (S)-5-((tert-butoxycarbonyl)amino)-2-(5-(((2,4-diaminopteridin-6-yl)methyl)(methyl) amino)thiophene-2-carboxamido)pentanoate (XC7)

Under a nitrogen atmosphere: to a solution of methylated amine XC6 in dry DMA (2 mL) was added 6-(bromomethyl)pteridine-2,4-diamine/2-propanol (1:1) (68.2 mg, 0.216 mmol) (WO 98/024789) and CaCO₃ (47.7 mg, 0.476 mmol). The flask was wrapped in tinfoil and the mixture was stirred for 6 days at RT. The mixture was filtered over Celite®, rinsed with DMA and concentrated in vacuo. The crude product was purified by flash chromatography (silica gel, DCM:MeOH 1:0 to 8:2) to afford methyl ester XC7 (63 mg, 52%) as a brown solid.

¹H NMR (400 MHz, CD₃OD) δ (ppm) 8.67 (s, 1H), 7.54 (d, J=4.3 Hz, 1H), 6.05 (d, J=4.4 Hz, 1H), 4.78-4.73 (m, 2H), 4.51 (br dd, J=9.1, 4.9 Hz, 1H), 3.73 (s, 3H), 3.21 (s, 3H), 3.08 (t, J=6.8 Hz, 2H), 1.98-1.87 (m, 1H), 1.84-1.72 (m, 1H), 1.66-1.50 (m, 2H), 1.43 (s, 9H).

MS (ESI⁺) calc. for C₂₄H₃₄N₉O₅S⁺ [M+H]⁺ 560.2, found 560.5.

### Methyl (S)-5-(4-amino-2-(2H-tetrazol-5-yl)benzamido)-2-(5-(((2,4-diaminopteridin-6-yl) methyl)(methyl)amino)thiophene-2-carboxamido)pentanoate (XC8)

To a stirred suspension of methyl ester XC7 (63 mg, 0.113 mmol) in dioxane (2 mL) was added slowly 4N HCl in dioxane (2 mL, 8.00 mmol) and the resulting mixture was stirred for 2 h at RT. The solids were filtered off and rinsed with Et₂O, dried under high vacuum, affording a brown powder. To a cooled (ice-bath) mixture of the obtained HCl salt in DMF (2 mL) were subsequently added a solution of XX21 (0.39 M in DMF, 0.292 mL, 0.113 mmol), HATU (51.4 mg, 0.135 mmol) and DIPEA (0.118 mL, 0.675 mmol) and the resulting mixture was stirred for 15 min at 4° C. Then, after concentration in vacuo, MeCN was added (8 mL) and stirred for 30 min, solids were filtered off, rinsed with MeCN and dried under high vacuum to give amine XC8 in a quantitative yield.

MS (ESI⁺) calc. for C₂₇H₃₁N₁₄O₄S⁺ [M+H]⁺ 647.2, found 647.5.

### (S)-5-(4-Amino-2-(2H-tetrazol-5-yl)benzamido)-2-(5-(((2,4-diaminopteridin-6-yl)methyl) (methyl)amino)thiophene-2-carboxamido)pentanoic acid (XC9)

Saponification of methyl ester XC8 (37 mg, 0.06 mmol) was carried out according to general procedure XXE. The crude solid was purified by preparative RP-HPLC (water×0.10% TFA/MeCN, gradient 5% to 35%). The combined product fractions were concentrated in vacuo to remove MeCN. The aqueous solution was lyophilized to yield compound XC9 (4.7 mg, 13%) as an orange solid.

¹H NMR (400 MHz, DMSO-d6) δ (ppm) 12.51 (br s, 1H), 9.15 (br s, 1H), 8.92 (br s, 1H), 8.78 (s, 1H), 8.46 (br s, 1H), 8.22-8.09 (m, 2H), 7.56 (br d, J=4.1 Hz, 2H), 7.44 (br d, J=9.0 Hz, 1H), 6.73-6.66 (m, 2H), 6.56 (br s, 2H), 6.04 (d, J=4.3 Hz, 1H), 4.76 (s, 2H), 4.29-4.20 (m, 1H), 3.21 (s, 3H), 3.12-3.06 (m, 2H), 2.89 (br s, 1H), 1.82-1.73 (m, 1H), 1.70-1.62 (m, 1H), 1.55-1.43 (m, 2H).

MS (ESI⁺) calc. for C₂₆H₂₉N₁₄O₄S⁺ [M+H]⁺ 633.2, found 633.4.

### Preparation of (S)-5-(4-amino-2-(1H-tetrazol-5-yl)benzamido)-2-(5-(((2,4-diamino-5-chloroquinazolin-6-yl)methyl)amino)thiophene-2-carboxamido)pentanoic acid (XT126)

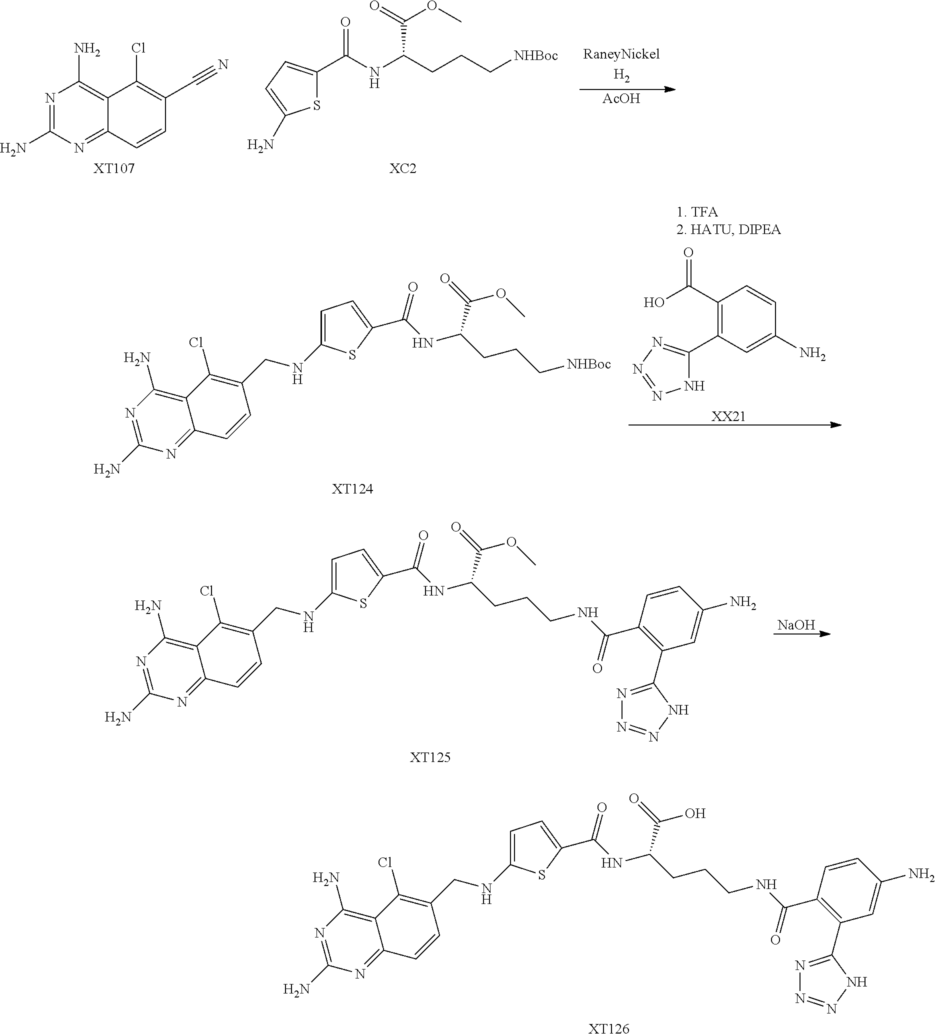

### Methyl (S)-5-((tert-butoxycarbonyl)amino)-2-(5-(((2,4-diamino-5-chloroquinazolin-6-yl) methyl)amino)thiophene-2-carboxamido)pentanoate (XT124)

To a solution of aniline XC2 (203 mg, 0.55 mmol) and nitrile XT107 (100 mg, 0.46 mmol) in AcOH (5 mL) was added a slurry of Raney Nickel 50% in water (0.5 mL). The resulting suspension was briefly purged with hydrogen before being put under hydrogen atmosphere and stirred for 1 day at RT. The reaction mixture was then filtered over Celite® and the cake rinsed with AcOH until the eluting filtrate turned colorless. Next, the filtrate was diluted with toluene and concentrated in vacuo. The crude product was purified by flash chromatography (silica gel, DCM:MeOH 1:0 to 8:2) to afford carbamate XT124 (11 mg, 4%) as a yellow solid.

MS (ESI⁺) calc. for C₂₅H₃₃ClN₇O₅S⁺ [M+H]⁺ 578.2, found 578.5.

### Methyl (S)-5-(4-amino-2-(1H-tetrazol-5-yl)benzamido)-2-(5-(((2,4-diamino-5-chloroquinazolin-6-yl)methyl)amino)thiophene-2-carboxamido)pentanoate (XT125)

According to general procedure XXD, carbamate XT124 (100 mg, 0.18 mmol) was reacted with a solution of XX21 (0.39 M, 455 μL, 0.18 mmol) in DMF to afford methyl ester XT125 (8 mg, 63%) as a light brown solid.

MS (ESI⁺) calc. for C₂₈H₃₀ClN₁₂O₄S⁺ [M+H]⁺ 665.2, found 665.5.

### (S)-5-(4-Amino-2-(1H-tetrazol-5-yl)benzamido)-2-(5-(((2,4-diamino-5-chloroquinazolin-6-yl)methyl)amino)thiophene-2-carboxamido)pentanoic acid (XT126)

Saponification of methyl ester XT125 (56 mg, 0.09 mmol) was carried out according to general procedure XXE, using NaOH instead of LiOH. The crude solid was purified by preparative RP-HPLC (water×0.1% TFA/MeCN, gradient 5% to 35%). Product fractions were pooled, MeCN was removed by rotary evaporation, and the aqueous solution was lyophilized to yield compound XT126 (0.9 mg, 11%) as a white solid.

MS (ESI⁺) calc. for C₂₇H₂₈ClN₁₂O₄S⁺ [M+H]⁺ 651.2, found 651.3.

### Preparation of (S)-5-(4-amino-2-(1H-tetrazol-5-yl)benzamido)-2-(4-(2-(2,4-diaminopteridin-6-yl)ethyl)benzamido)pentanoic acid (XT128)

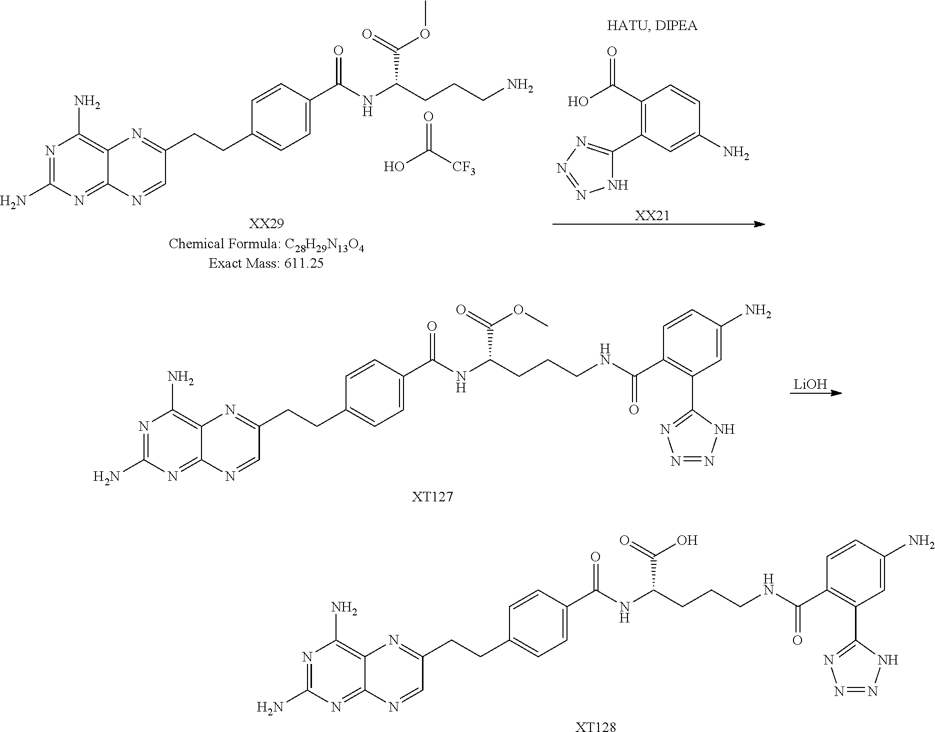

### Methyl (S)-5-(4-amino-2-(1H-tetrazol-5-yl)benzamido)-2-(4-(2-(2,4-diaminopteridin-6-yl) ethyl)benzamido)pentanoate (XT127)

Using general procedure XXA, aniline XX29 (100 mg, 0.18 mmol) was reacted with a solution of XX21 (0.39 M, 470 μL, 0.18 mmol) in DMF. Purification by trituration from MeCN afforded methyl ester XT127 (quant) as a brown solid.

MS (ESI) calc. for C₂₉H₃₂N₁₃O₄⁺ [M+H]⁺ 626.3, found 626.5.

### (S)-5-(4-Amino-2-(1H-tetrazol-5-yl)benzamido)-2-(4-(2-(2,4-diaminopteridin-6-yl)ethyl) benzamido)pentanoic acid (XT128)

Saponification of methyl ester XT127 (113 mg, 0.18 mmol) was carried out according to general procedure XXE. The crude solid was purified by preparative RP-HPLC (water×0.1% TFA/MeCN, gradient 5% to 35%). Product fractions were pooled, MeCN was removed by rotary evaporation, and the aqueous solution was lyophilized to yield compound XT128 (24 mg, 22%) as a white solid.

¹H NMR (400 MHz, DMSO-d₆) ppm=13.03-12.18 (m, 2H), 9.26 (s, 1H), 9.17 (s, 1H), 8.76 (s, 1H), 8.67-8.54 (m, 1H), 8.51 (br d, J=7.8 Hz, 1H), 8.16 (br t, J=5.2 Hz, 1H), 7.81 (d, J=8.3 Hz, 2H), 7.69-7.51 (m, 1H), 7.51-7.41 (m, 1H), 7.36 (d, J=8.3 Hz, 2H), 6.80-6.66 (m, 2H), 4.42-4.30 (m, 1H), 3.30-3.18 (m, 4H), 3.18-3.07 (m, 2H), 1.90-1.69 (m, 2H), 1.63-1.45 (m, 2H).

MS (ESI⁺) calc. for C₂₈H₃₀N₁₃O₄⁺ [M+H]⁺ 612.3, found 612.6.

### Preparation of (S)-5-(4-amino-2-(1H-tetrazol-5-yl)benzamido)-2-(4-(((2,4-diaminopteridin-6-yl)methyl)amino)-3-methoxybenzamido)pentanoic acid (XT132)

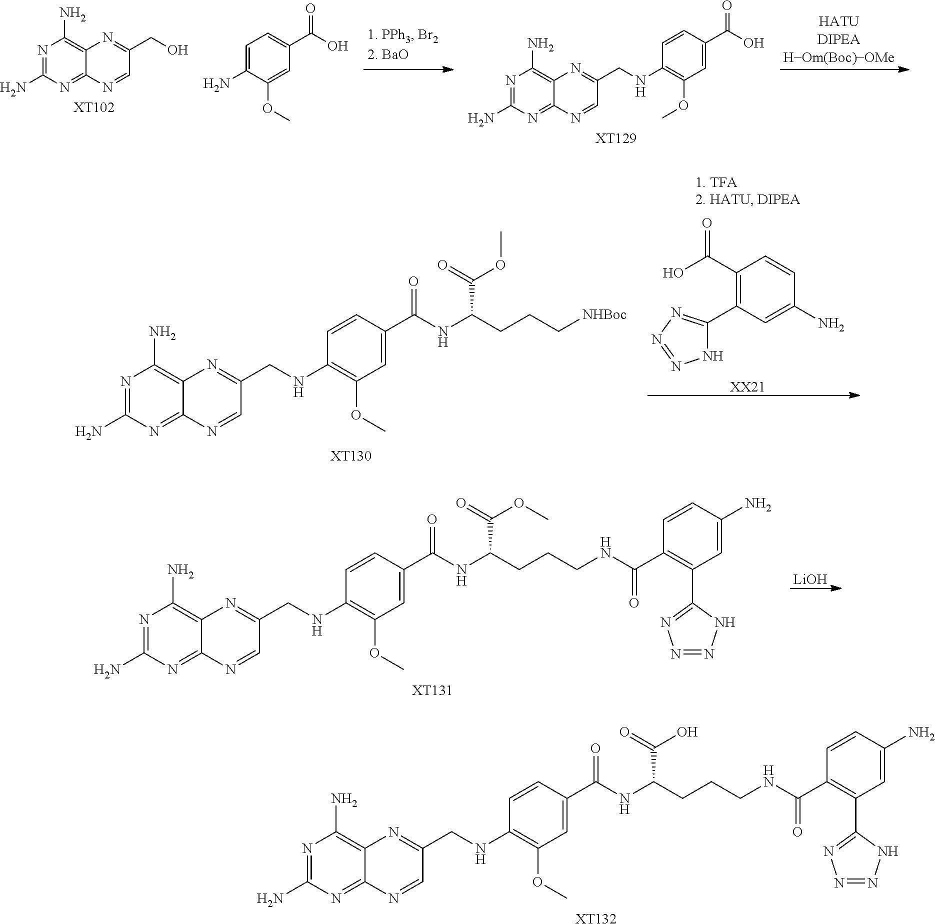

### 4-(((2,4-Diaminopteridin-6-yl)methyl)amino)-3-methoxybenzoic acid (XT129)

To a cooled solution of PPh₃ (2.0 g, 7.8 mmol) in DMA (6.1 mL) was added bromine (0.4 mL, 7.8 mmol) over a 15 min period. The resulting mixture was stirred for 30 min at 0° C., then alcohol XT102 (0.5 g, 2.6 mmol) was added and stirred for 17 h at RT. To the crude bromide, 4-amino-3-methoxybenzoic acid (0.5 g, 3.3 mmol) was added, followed by barium oxide (0.6 g, 3.3 mmol) and the resulting mixture was warmed at 55° C. for 24 h. Afterwards, the crude mixture was cooled to RT and diluted with DCM (72.5 mL) and MeOH (2.5 mL). The obtained suspension was filtered and the residue taken up in water (30 mL), the resulting suspension was stirred at RT for 30 min and subsequently filtered. The residue was then suspended in MeCN (30 mL), stirred for 15 min and subsequently filtered. The obtained residue was washed with ether (5 mL) and dried to afford carboxylic acid XT129 (0.43 g, 48%).

¹H NMR (400 MHz, DMSO-d₆) ppm=12.49-11.99 (m, 1H), 9.16 (br s, 1H), 9.10 (br s, 1H), 8.77 (s, 1H), 8.58-8.20 (m, 1H), 7.53-7.19 (m, 1H) 7.41 (br d, J=8.3 Hz, 1H), 7.33 (d, J=1.3 Hz, 1H), 6.63 (d, J=8.3 Hz, 1H), 6.58 (br t, J=6.0 Hz, 1H), 4.67-4.58 (m, 2H), 3.87 (s, 3H).

MS (ESI⁺) calc. for C₁₅H₁₆N₇O₃⁺ [M+H]⁺ 342.1, found 342.3.

### Methyl (S)-5-((tert-butoxycarbonyl)amino)-2-(4-(((2,4-diaminopteridin-6-yl)methyl)amino)-3-methoxybenzamido)pentanoate (XT130)

To a cooled suspension of carboxylic acid XT129 (200 mg, 0.59 mmol) and L-Orn(Boc)-OMe·HCl (182 mg, 0.65 mmol) in DMF (6.7 mL) was added HATU (267 mg, 0.70 mmol) and DIPEA (1.0 mL, 5.86 mmol). The resulting mixture was stirred for 45 min at RT and then concentrated in vacuo. The crude product was purified by flash chromatography (silica gel, DCM/MeOH 1:0 to 9:1) to afford carbamate XT130 (248 mg, 74%) as a yellow/orange solid.

¹H NMR (400 MHz, DMSO-d₆) ppm=8.67 (s, 1H), 8.29 (d, J=7.4 Hz, 1H), 7.95 (s, 1H), 7.88-7.67 (m, 2H), 7.41-7.36 (m, 1H), 7.35 (d, J=1.8 Hz, 1H), 6.82-6.68 (m, 2H), 6.61 (d, J=8.3 Hz, 1H), 6.38-6.29 (m, 1H), 4.58-4.50 (m, 2H), 4.41-4.30 (m, 1H), 3.87 (s, 3H), 3.61 (s, 3H), 2.97-2.88 (m, 2H), 1.83-1.65 (m, 2H), 1.55-1.37 (m, 2H), 1.36 (s, 9H).

MS (ESI⁺) calc. for C₂₆H₃₆N₉O₆⁺ [M+H]⁺ 570.3, found 570.4.

### Methyl (S)-5-(4-amino-2-(1H-tetrazol-5-yl)benzamido)-2-(4-(((2,4-diaminopteridin-6-yl) methyl)amino)-3-methoxybenzamido)pentanoate (XT131)

Following general procedure XXD, carbamate XT130 (50 mg, 0.09 mmol) was reacted with a solution of XX21 (0.39 M, 228 μL, 0.09 mmol) in DMF to afford methyl ester XT131 (58 mg, quant) as a brown solid.

MS (ESI⁺) calc. for C₂₉H₃₃N₁₄O₅⁺ [M+H]⁺ 657.3, found 657.7.

### (S)-5-(4-Amino-2-(1H-tetrazol-5-yl)benzamido)-2-(4-(((2,4-diaminopteridin-6-yl)methyl) amino)-3-methoxybenzamido)pentanoic acid (XT132)

Saponification of methyl ester XT131 (113 mg, 0.18 mmol) was carried out according to general procedure XXE. The crude solid was purified by preparative RP-HPLC (water×0.1% TFA/MeCN, gradient 5% to 35%). Product fractions were pooled, MeCN was removed by rotary evaporation, and the aqueous solution was lyophilized to yield compound XT132 (14 mg, 27%) as a yellow solid.

¹H NMR (400 MHz, DMSO-d₆) ppm=12.54-12.33 (m, 1H), 9.26 (br s, 1H), 9.22 (br s, 1H), 8.77 (s, 1H), 8.68-8.48 (m, 1H), 8.20 (d, J=7.8 Hz, 1H), 8.17-8.10 (m, 1H), 7.55-7.34 (br s, 1H), 7.49-7.41 (m, 1H), 7.41-7.35 (m, 2H), 6.70 (br d, J=6.6 Hz, 2H), 6.60 (d, J=8.8 Hz, 1H), 6.44-6.33 (m, 1H), 4.68-4.58 (m, 2H), 4.41-4.28 (m, 1H), 3.88 (s, 3H), 3.15-3.05 (m, 2H), 1.88-1.64 (m, 2H), 1.61-1.41 (m, 2H).

MS (ESI⁺) calc. for C₂₈H₃₁N₁₄O₅⁺ [M+H]⁺ 643.3, found 643.5.

### Preparation of 4-((S)-2-((S)-2-((tert-butoxycarbonyl)amino)-3-methylbutanamido) propanamido)-2-(1H-tetrazol-5-yl)benzoic acid (XT140)

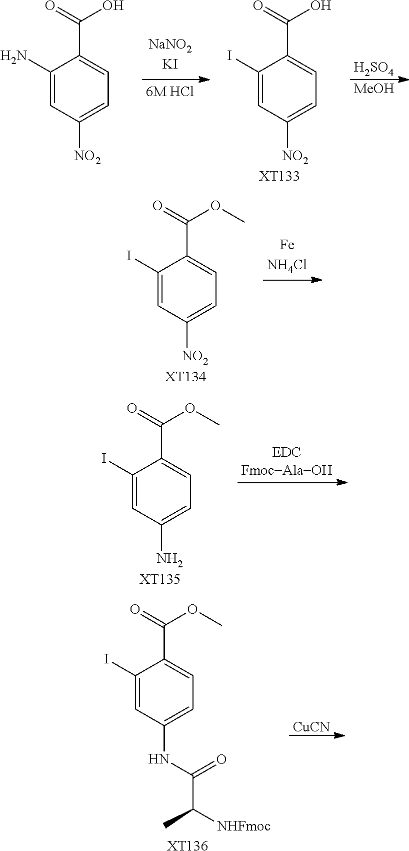

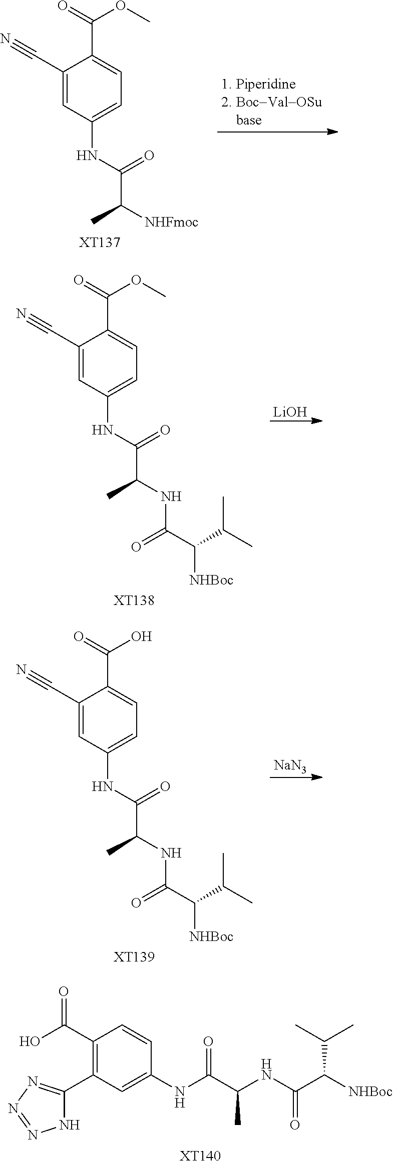

### 2-Iodo-4-nitrobenzoic acid (XT133)

A 500 mL round-bottom flask equipped with a mechanical stirrer was charged with 2-amino-4-nitrobenzoic acid (12 g, 65.9 mmol), concentrated HCl (12 M, 160 mL, 1920 mmol) and water (160 mL) forming an orange suspension. The mixture was cooled to 0° C. followed by the dropwise addition of sodium nitrite (9.09 g, 132 mmol) dissolved in water (80 mL), keeping the internal temperature below 5° C. during the addition. The resulting solution was stirred at 0° C. for 2 h followed by the addition of an aqueous solution of potassium iodide (65.6 g, 395 mmol) in water (80 mL) over the course of 1.5 h. The reaction mixture was stirred at 0° C. for 60 min and at RT for 20 min. Next, 150 mL of EtOAc was added followed by sat. aq. Na₂SO₃ until the color of the solution changed from dark brown to pale orange. The layers were separated and the aqueous layer was additionally extracted with EtOAc (2×150 mL). The combined organic layers were washed with brine (250 mL), dried over anhydrous Na₂SO₄, filtered and concentrated affording carboxylic acid XT133 (18 g, 93%) as a yellow solid.

¹H NMR (400 MHz, DMSO-d₆) δ ppm=7.87 (d, J=8.50 Hz, 1H), 8.28 (dd, J=8.50, 2.25 Hz, 1H), 8.65 (d, J=2.25 Hz, 1H), 13.95 (br s, 1H).

MS (ESI⁻) calc. for C₇H₃INO₄⁻ [M−H]⁻ 291.9, found 292.0.

### Methyl 2-iodo-4-nitrobenzoate (XT134)

To a solution of carboxylic acid XT133 (18 g, 61.4 mmol) in MeOH (150 mL) was added H₂SO₄ (3.3 mL, 61.4 mmol) and the resulting mixture was heated to 85° C. for 16 h. Afterwards, the crude reaction mixture was cooled to RT and concentrated. The obtained residue was taken up in Et₂O (600 mL), washed with 1 M HCl (300 mL), water (300 mL), saturated aqueous NaHCO₃ solution (300 mL), water (200 mL), and brine (250 mL). The organic phase was dried with Na₂SO₄, filtered and the filtrate concentrated to yield methyl ester XT134 (14.2 g, 75%) as a yellow solid.

¹H NMR (400 MHz, DMSO-d₆) δ ppm=8.67 (d, J=2.3 Hz, 1H), 8.31 (dd, J=8.5, 2.3 Hz, 1H), 7.90 (d, J=8.5 Hz, 1H), 3.92 (s, 3H).

### Methyl 4-amino-2-iodobenzoate (XT135)

A round-bottom flask equipped with a mechanical stirred was loaded with iron (7.75 g, 139 mmol) and ammonium chloride (24.7 g, 462 mmol), suspended in ethanol (200 mL) and water (20 mL). Next, powdered methyl ester XT134 (14.2 g, 46.2 mmol) was added and the resulting suspension was heated to 80° C. for 16 h. Then, additional iron (7.75 g, 139 mmol) and ammonium chloride (24.7 g, 462 mmol) were added and stirring at 80° C. was continued for another 2 h. Afterwards, the mixture was cooled to RT, filtered over Celite® and concentrated. The crude residue was taken up in EtOAc (200 mL), washed with water (100 mL) and the aqueous phase was back extracted twice with EtOAc (150 mL). The combined organic layers were washed with brine (150 mL) and dried over MgSO₄, filtered and concentrated. The crude product was purified by flash chromatography (silica gel, DCM) to afford aniline XT135 (9 g, 70%) as a white solid.

¹H NMR (400 MHz, DMSO-d₆) δ ppm=7.67-7.54 (m, 1H), 7.30-7.12 (m, 1H), 6.57 (dd, J=8.6, 2.3 Hz, 1H), 6.05 (s, 2H), 3.72 (s, 3H).

MS (ESI⁺) calc. for C₈H₉INO₂⁺ [M+H]⁺ 278.0, found 278.0.

### Methyl (S)-4-(2-((((9H-fluoren-9-yl)methoxy)carbonyl)amino)propanamido)-2-iodobenzoate (XT136)

To a cooled suspension of aniline XT135 (8.6 g, 31.0 mmol) and Fmoc-Ala-OH (10.2 g, 32.6 mmol) in DCM (160 mL) was added EDC (6.3 g, 32.6 mmol) and the resulting mixture was stirred for 6 h at RT. Then another portion of Fmoc-Ala-OH (2.5 g, 8.2 mmol) and EDC 1.6 g, 8.2 mmol) were added and the mixture was stirred overnight. Next, the crude reaction mixture was concentrated to a volume of ˜40 mL and the crude product solution was purified by flash chromatography (silica gel, DCM:EtOAc, 1:0 to 8:2) to obtain amide XT136 (11.9 g, 67%) as a white solid.

¹H NMR (400 MHz, DMSO-d₆) δ ppm=10.43-10.29 (m, 1H), 8.41 (d, J=1.5 Hz, 1H), 8.01-7.84 (m, 2H), 7.84-7.67 (m, 5H), 7.48-7.37 (m, 2H), 7.37-7.25 (m, 2H), 4.43-4.27 (m, 2H), 4.27-4.14 (m, 2H), 3.82 (s, 3H), 1.33 (d, J=7.1 Hz, 3H).

MS (ESI⁺) calc. for C₂₆H₂₄IN₂O₅⁺ [M+H]⁺ 571.1, found 571.2.

### Methyl (S)-4-(2-((((9H-fluoren-9-yl)methoxy)carbonyl)amino)propanamido)-2-cyano benzoate (XT137)

A round-bottom flask loaded with iodide XT136 (10.7 g, 18.8 mmol) and copper cyanide (3.4 g, 37.5 mmol) was first purged with nitrogen followed by addition of DMF (50 mL) and the resulting mixture was heated to 80° C. for 2 h. Then, the mixture was cooled to RT, diluted with EtOAc (350 mL) and water (200 mL) and washed with saturated aqueous NaHCO₃ (350 mL). The aqueous phase was extracted with EtOAc (200 mL) and the combined organic layers were washed with brine (200 mL) and dried over Na₂SO₄. The solution was filtered and concentrated to obtain nitril XT137 (quant) and used in the next step without further purification.

¹H NMR (400 MHz, DMSO-d₆) δ ppm=10.66 (s, 1H), 8.23 (d, J=2.1 Hz, 1H), 8.11 (d, J=8.8 Hz, 1H), 7.99-7.92 (m, 2H), 7.89 (d, J=7.5 Hz, 2H), 7.79 (d, J=6.9 Hz, 1H), 7.73 (t, J=6.9 Hz, 2H), 7.45-7.39 (m, 2H), 7.34 (br d, J=7.3 Hz, 2H), 4.29 (s, 2H), 4.24-4.13 (m, 2H), 3.89 (s, 3H), 1.33 (d, J=7.1 Hz, 3H).

MS (ESI⁺) calc. for C₂₇H₂₄N₃O₅⁺ [M+H]⁺ 470.2, found 470.3.

### Methyl 4-((S)-2-((S)-2-((tert-butoxycarbonyl)amino)-3-methylbutanamido)propanamido)-2-cyanobenzoate (XT138)

To a solution of nitril XT137 (8.8 g, 18.8 mmol) in THF (185 mL) was added piperidine (18.6 mL, 188 mmol) and the resulting mixture was stirred for 1 h at RT. Then, the mixture was diluted with toluene (400 mL) and concentrated. The solid residue was taken up in water (300 mL) and a solution of Boc-Val-OSu (5.9 g, 18.8 mmol) in THF (300 mL) was added followed by NaHCO₃ (3.2 g, 37.5 mmol) and the resulting mixture was stirred for 18 h at RT. Next, THF was evaporated and the aqueous residue was extracted with EtOAc (500 mL). The organic layer was washed with brine (300 mL), dried over Na₂SO₄, filtered and concentrated. The crude product was purified by flash chromatography (silica gel, DCM:EtOAc, 1:0 to 3:1) to obtain dipeptide XT138 (8.4 g, quant) as a white solid.

¹H NMR (400 MHz, DMSO-d₆) δ ppm=10.74-10.53 (m, 1H), 8.26-8.14 (m, 2H), 8.14-8.06 (m, 1H), 8.00-7.83 (m, 1H), 6.77-6.57 (m, 1H), 4.40 (br d, J=6.8 Hz, 1H), 3.89 (s, 3H), 3.87-3.81 (m, 1H), 2.02-1.88 (m, 1H), 1.46-1.28 (m, 12H), 0.91-0.75 (m, 6H).

MS (ESI⁺) calc. for C₂₂H₃₁N₄O₆⁺ [M+H]⁺ 447.2, found 447.3.

### 4-((S)-2-((S)-2-((tert-Butoxycarbonyl)amino)-3-methylbutanamido)propanamido)-2-cyanobenzoic acid (XT139)

To a solution of methyl ester XT138 (8.5 g, 19.0 mmol) in THF (120 mL) was added a solution of LiOH (0.9 g, 38.1 mmol) in water (60 mL) and the resulting mixture was stirred for 2 h at RT. Next, the solution was acidified using a 0.5 M KHSO₄ solution (175 mL) and extracted twice with EtOAc (350 mL). The combined organic layer was washed with brine (225 mL), dried over Na₂SO₄, filtered and concentrated to obtain carboxylic acid XT139 (7.2 g, 83%) as a white solid.

¹H NMR (400 MHz, DMSO-d₆) δ ppm=13.78-13.35 (m, 1H), 10.60 (s, 1H), 8.21 (br d, J=6.5 Hz, 1H), 8.17 (d, J=2.1 Hz, 1H), 8.08 (d, J=8.8 Hz, 1H), 7.90 (dd, J=8.7, 2.1 Hz, 1H), 6.68 (br d, J=8.8 Hz, 1H), 4.49-4.33 (m, 1H), 3.89-3.77 (m, 1H), 1.99-1.90 (m, 1H), 1.38 (s, 9H), 1.33 (d, J=7.1 Hz, 3H), 0.93-0.79 (m, 6H).

MS (ESI⁺) calc. for C₂₁H₂₉N₄O₆⁺ [M+H]⁺ 433.2, found 433.3.

### 4-((S)-2-((S)-2-((tert-Butoxycarbonyl)amino)-3-methylbutanamido)propanamido)-2-(1H-tetrazol-5-yl)benzoic acid (XT140)

A reaction vial loaded with nitril XT139 (500 mg, 1.16 mmol) and sodium azide (225 mg, 3.47 mmol) was purged with nitrogen, followed by addition of DMF (5 mL) and the resulting suspension was heated to 85° C. for 18 h. Afterwards, the reaction mixture was cooled to RT, filtered over Celite® and the filtrate was concentrated. The crude product was suspended in Et₂O (20 mL), stirred for 10 min and filtered. The obtained residue was suspended in MeCN (20 mL), stirred for 30 min and filtered (3 cycles). The obtained solid was dried on air to yield tetrazole XT140 (450 mg, 82%) as a white solid.

¹H NMR (400 MHz, DMSO-d₆) δ ppm=10.04-9.77 (m, 1H), 8.05 (br d, J=6.8 Hz, 1H), 7.86 (br s, 1H), 7.51 (br d, J=7.1 Hz, 1H), 7.17 (br d, J=7.8 Hz, 1H), 6.72 (br d, J=8.4 Hz, 1H), 4.54-4.39 (m, 1H), 3.94-3.79 (m, 1H), 2.02-1.88 (m, 1H), 1.38 (s, 9H), 1.31 (br d, J=6.8 Hz, 3H), 0.91-0.77 (m, 6H).

MS (ESI⁺) calc. for C₂₁H₃₀N₇O₆⁺ [M+H]⁺ 476.2, found 476.3.

### Preparation of (S)-2-(4-(2-(2,4-diamino-5-chloroquinazolin-6-yl)ethyl)benzamido)-5-(4-((S)-2-((S)-2-(6-(2,5-dioxo-2,5-dihydro-1H-pyrrol-1-yl)hexanamido)-3-methylbutanamido) propanamido)-2-(1H-tetrazol-5-yl)benzamido)pentanoic acid (XT143)

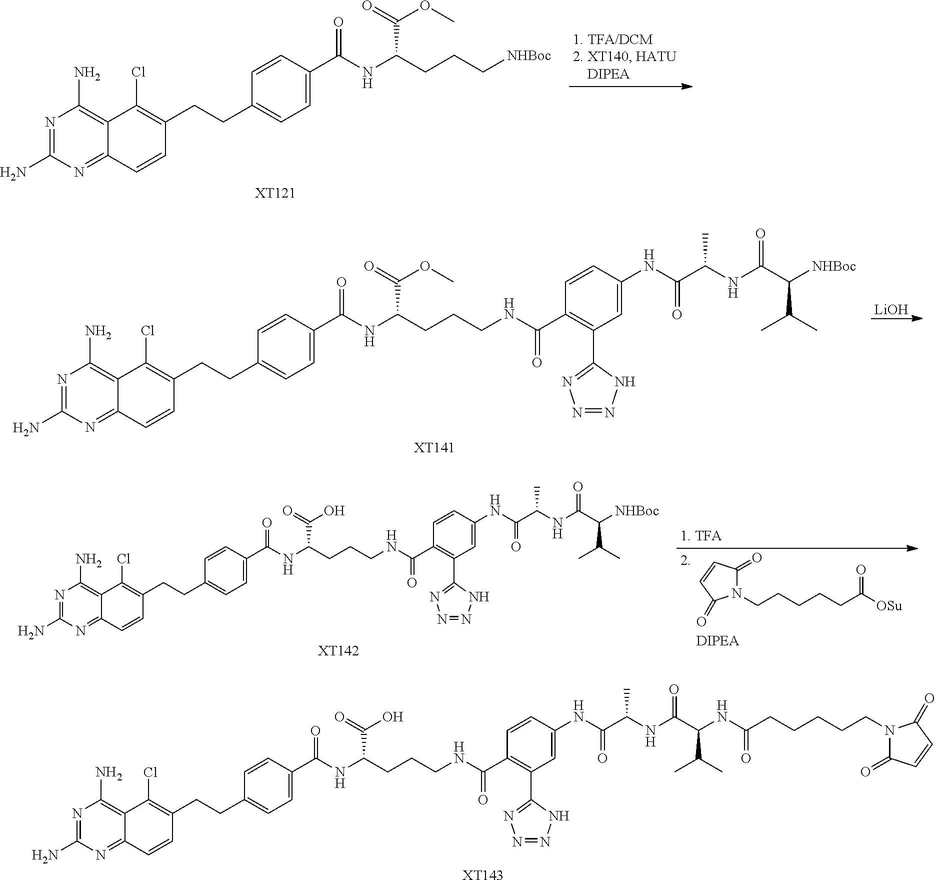

### Methyl (S)-5-(4-((S)-2-((S)-2-((tert-butoxycarbonyl)amino)-3-methylbutanamido) propanamido)-2-(JH-tetrazol-5-yl)benzamido)-2-(4-(2-(2,4-diamino-5-chloroquinazolin-6-yl)ethyl)benzamido)pentanoate (XT141)

Following general procedure XXD, carbamate XT121 (2200 mg, 3.85 mmol) was deprotected and obtained as a white solid. A portion of the TFA-salt (600 mg, 1.03 mmol) was reacted with XT140 (439 mg, 0.93 mmol) in DMF to afford methyl ester XT141 (520 mg, 61%) as a light brown solid.

¹H NMR (400 MHz, DMSO-d₆) ppm=12.75 (br d, J=5.3 Hz, 1H), 10.31 (br s, 1H), 9.19 (s, 1H), 8.61 (br d, J=7.4 Hz, 1H), 8.54 (s, 1H), 8.43-8.31 (m, 1H), 8.11 (br d, J=6.4 Hz, 1H), 8.01-7.78 (m, 3H), 7.79-7.68 (m, 3H), 7.64 (d, J=8.6 Hz, 1H), 7.62-7.53 (m, 1H), 7.27 (d, J=8.3 Hz, 3H), 6.65 (br d, J=8.8 Hz, 1H), 4.42-4.29 (m, 2H), 3.80-3.73 (m, 1H), 3.58 (s, 3H), 3.15-2.98 (m, 4H), 2.95-2.82 (m, 2H), 1.98-1.65 (m, 3H), 1.58-1.40 (m, 2H), 1.35-1.20 (m, 12H), 0.81 (s, 6H).

MS (ESI⁺) calc. for C₄₄H₅₅ClN₁₃O₈⁺ [M+H]⁺ 928.4, found 928.8.

### (S)-5-(4-((S)-2-((S)-2-((tert-Butoxycarbonyl)amino)-3-methylbutanamido)propanamido)-2-(1H-tetrazol-5-yl)benzamido)-2-(4-(2-(2,4-diamino-5-chloroquinazolin-6-yl)ethyl) benzamido)pentanoic acid (XT142)

Saponification of methyl ester XT141 (500 mg, 0.54 mmol) was performed according to general procedure XXE. Afterwards, the residue was suspended in water (50 mL), stirred for 15 min and filtered. The obtained solid was subsequently washed with water (20 mL), MeCN (2×5 mL) and Et₂O (25 mL) to obtain carboxylic acid XT142 (390 mg, 79%) as a light brown solid.

¹H NMR (400 MHz, DMSO-d₆) ppm=13.28-11.94 (m, 1H), 10.30 (s, 1H), 9.16 (br s, 1H), 8.64-8.45 (m, 2H), 8.40 (br s, 1H), 8.11 (br d, J=6.6 Hz, 1H), 8.01-7.67 (m, 6H), 7.67-7.53 (m, 2H), 7.26 (dd, J=8.4, 2.7 Hz, 3H), 6.65 (br d, J=8.8 Hz, 1H), 6.57-6.45 (m, 1H), 4.44-4.24 (m, 2H), 3.76 (br s, 1H), 3.16-2.97 (m, 4H), 2.95-2.82 (m, 2H), 1.95-1.63 (m, 3H), 1.62-1.41 (m, 2H), 1.39-1.20 (m, 12H), 0.92-0.62 (m, 6H).

MS (ESI⁺) calc. for C₄₃H₅₃ClN₁₃O₈⁺ [M+H]⁺ 914.4, found 914.7.

### (S)-2-(4-(2-(2,4-Diamino-5-chloroquinazolin-6-yl)ethyl)benzamido)-5-(4-((S)-2-((S)-2-(6-(2,5-dioxo-2,5-dihydro-1H-pyrrol-1-yl)hexanamido)-3-methylbutanamido)propanamido)-2-(1H-tetrazol-5-yl)benzamido)pentanoic acid (XT143)

Following the procedure of XT114, carbamate XT142 (390 mg, 0.38 mmol) is deprotected and subsequently coupled with 6-maleimidohexanoic acid N-hydroxylsuccinimide ester (118 mg, 0.38 mmol). Purification of the crude product by RP-HPLC (water×0.1% TFA/MeCN, gradient 25% to 32.5%) afforded XT143 (90 mg, 21%) as a white solid.

¹H NMR (400 MHz, DMSO-d₆) ppm=12.93-12.31 (m, 1H), 10.23 (s, 1H), 9.14 (br s, 1H), 8.61-8.43 (m, 2H), 8.39 (br s, 1H), 8.19 (d, J=6.6 Hz, 1H), 7.95-7.52 (br s, 1H), 7.87 (br s, 1H), 7.80-7.67 (m, 5H), 7.65-7.52 (m, 2H), 7.30-7.22 (m, 3H), 6.93 (s, 2H), 6.49 (s, 1H), 4.29 (br d, J=6.8 Hz, 2H), 4.09 (br d, J=1.0 Hz, 1H), 3.30-3.25 (m, 2H), 3.14-2.96 (m, 4H), 2.88 (br d, J=8.4 Hz, 2H), 2.16-1.96 (m, 2H), 1.94-1.63 (m, 3H), 1.59-1.32 (m, 6H), 1.25 (d, J=7.1 Hz, 3H), 1.11 (br d, J=7.6 Hz, 2H), 0.77 (dd, J=18.0, 6.8 Hz, 6H).

MS (ESI⁺) calc. for C₄₈H₅₆ClN₁₄O₉⁺ [M+H]⁺ 1007.4, found 1008.1.

### Preparation of (S)-2-(4-(2-(2,4-diamino-5-fluoroquinazolin-6-yl)ethyl)benzamido)-5-(4-((S)-2-((S)-2-(6-(2,5-dioxo-2,5-dihydro-1H-pyrrol-1-yl)hexanamido)-3-methylbutanamido) propanamido)-2-(1H-tetrazol-5-yl)benzamido)pentanoic acid (XT151)

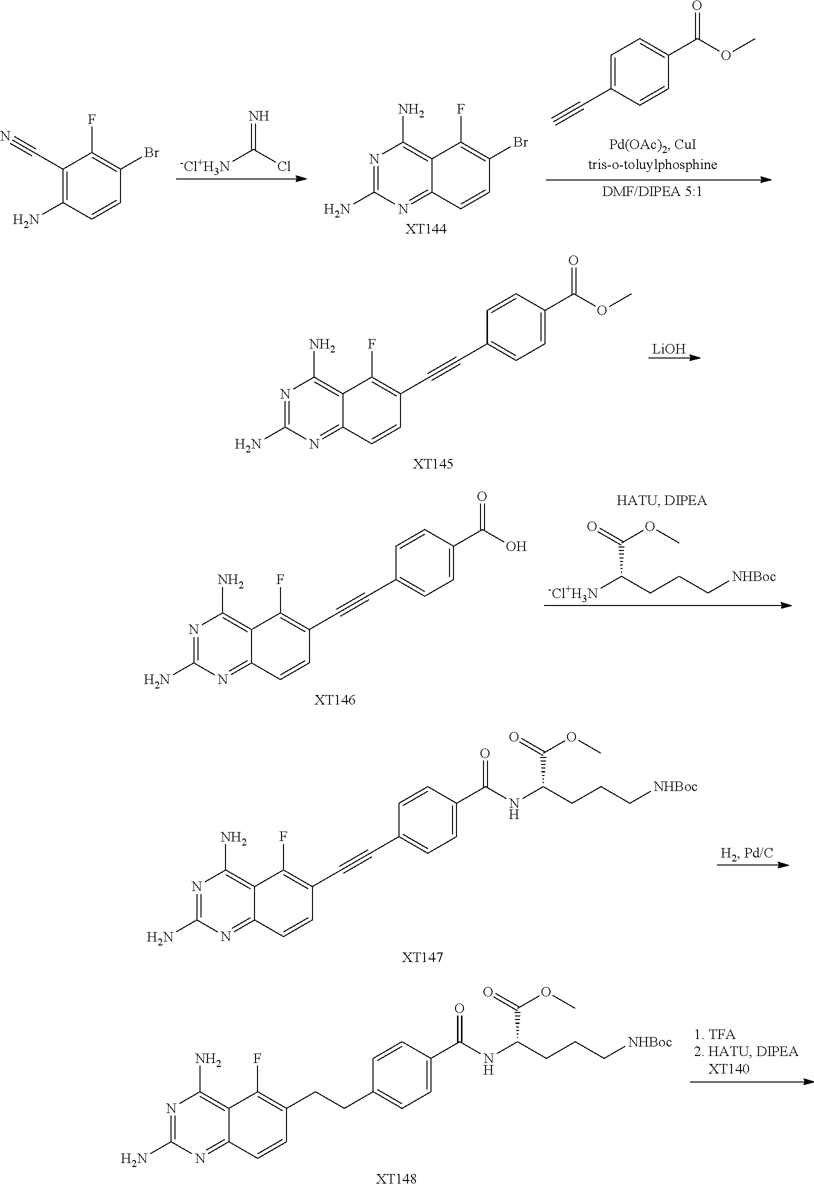

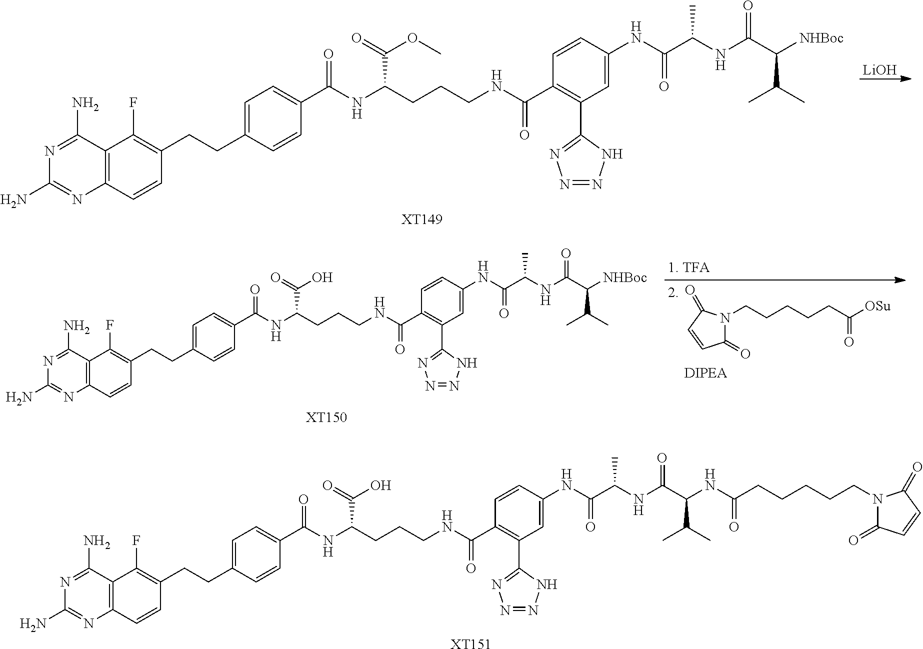

### 6-Bromo-5-fluoroquinazoline-2,4-diamine (XT144)

A 500 mL 3-necked round-bottom flask was loaded with carbamimidic chloride hydrochloride (10.2 g, 89 mmol) and 6-amino-3-bromo-2-fluorobenzonitrile (19.1 g, 89 mmol), the flask was purged with nitrogen (3 cycles) followed by addition of bis(2-methoxyethyl) ether (200 mL) under nitrogen atmosphere and the resulting mixture was gradually (over a 60 min period) warmed to 165° C. and stirred for another 3 h. Next, the mixture was cooled to RT, filtered and the solid residue was washed with Et₂O (150 mL) and dried under vacuum to yield bromide XT144 (11.2 g, 48.9% yield) as a light yellow solid.

MS (ESI⁺) calc. for C₈H₇BrFN₄⁺ [M+H]⁺ 257.0, found 257.1.

### Methyl 4-((2,4-diamino-5-fluoroquinazolin-6-yl)ethynyl)benzoate (XT145)

An argon purged 250 mL 2-necked flask was loaded with bromide XT144 (1000 mg, 3.89 mmol), methyl 4-ethynylbenzoate (685 mg, 4.28 mmol), Pd(OAc)₂ (44 mg, 0.20 mmol), tri-o-tolylphosphine (118 mg, 0.39 mmol) and copper(I) iodide (37 mg, 0.20 mmol). The flask was purged with nitrogen (3 cycles) followed by addition of DMF (50 mL) and DIPEA (10 mL). The resulting mixture was heated to 80° C. for a total of 11 days. After 3 days additional methyl 4-ethynylbenzoate (685 mg, 4.28 mmol) in DMF (2 mL) was added and after 5 days methyl 4-ethynylbenzoate (340 mg, 2.15 mmol) in DMF (1 mL) was added. Then, the reaction mixture was concentrated, the crude product was suspended in water (60 mL) and stirred for 15 min and filtered. The residue was washed with water (20 mL) and Et₂O (3×50 mL). The obtained solids were dried on air to yield methyl ester XT145 (1360 mg, quant) as a light brown solid.

MS (ESI⁺) calc. for C₁₈H₁₄FN₄O₂⁺ [M+H]⁺ 337.1, found 337.2.

### 4-((2,4-Diamino-5-fluoroquinazolin-6-yl)ethynyl)benzoic acid (XT146)

To a suspension of methyl ester XT145 (1.3 g, 3.9 mmol) in THF (30 mL) was added a solution of LiOH (0.47 g, 19.5 mmol) in water (15 mL) and the resulting mixture was stirred for 4 h at RT. Then, the mixture was cooled with an ice-bath followed by addition of acetic acid (2.23 mL, 38.9 mmol) and stirred for 15 min. Then, THF was evaporated and the resulting suspension was diluted with water (20 mL), filtered and the obtained residue was washed with MeCN (2×25 mL) and Et₂O (3×25 mL). The solid residue was dried overnight on air to yield carboxylic acid XT146 (1.32 g, quant) as a light brown solid.

MS (ESI⁺) calc. for C₁₇H₁₂FN₄O₂⁺ [M+H]⁺ 323.1, found 323.2.

### Methyl (S)-5-((tert-butoxycarbonyl)amino)-2-(4-((2,4-diamino-5-fluoroquinazolin-6-yl)ethynyl)benzamido)pentanoate (XT147)

To a cooled solution of carboxylic acid XT146 (1.25, 3.9 mmol) in DMA (20 mL) were subsequently added H-Orn(Boc)-OMe·HCl (1.15 g, 4.1 mmol), DIPEA (6.8 mL, 38.9 mmol) and HATU (1.55 g, 4.1 mmol) and the resulting mixture was stirred at RT for 30 min. Then, the reaction mixture was concentrated and the residue purified by flash chromatography (silica gel, DCM:MeOH, 1:0 to 8:2), the fractions containing product were concentrated and the residue was suspended in ether (50 mL), filtered and the solid was suspended in MeCN (25 mL), stirred for 10 min. and filtered. The residue was dried on air overnight to obtain alkyne XT147 (740 mg, 35% over 3 steps) as a yellow/brown solid.

MS (ESI⁺) calc. for C₂₈H₃₂FN₆O₅⁺ [M+H]⁺ 551.2, found 551.6.

### Methyl (S)-5-((tert-butoxycarbonyl)amino)-2-(4-(2-(2,4-diamino-5-fluoroquinazolin-6-yl) ethyl)benzamido)pentanoate (XT148)

A solution of alkyne XT147 (740 mg, 1.34 mmol) in AcOH (15 mL)/MeOH (3 mL) was put under a nitrogen atmosphere and Pd-C (500 mg) was added. The resulting suspension was purged with hydrogen (3×) and stirred under a hydrogen atmosphere for 2 h. Afterwards, the reaction mixture was filtered over Celite®, the cake was rinsed with MeOH (25 mL) and the obtained filtrate concentrated. The crude product was purified by flash chromatography (silica gel, DCM:MeOH, 1:0 to 4:1) to yield carbamate XT148 (640 mg, 86%) as an orange glass.

MS (ESI⁺) calc. for C₂₈H₃₆FN₆O₅⁺ [M+H]⁺ 555.3, found 555.5.

### Methyl (S)-5-(4-((S)-2-((S)-2-((tert-butoxycarbonyl)amino)-3-methylbutanamido) propanamido)-2-(JH-tetrazol-5-yl)benzamido)-2-(4-(2-(2,4-diamino-5-fluoroquinazolin-6-yl) ethyl)benzamido)pentanoate (XT149)

Following general procedure XXD, carbamate XT148 (640 mg, 1.15 mmol) was deprotected and split in 2 portions. One portion of the TFA-salt (300 mg, 0.53 mmol) was reacted with XT140 (226 mg, 0.48 mmol) in DMF to afford methyl ester XT149 (quant) as a light brown solid.

MS (ESI⁺) calc. for C₄₄H₅₅FN₁₃O₈⁺ [M+H]⁺ 912.4, found 913.1.

### (S)-5-(4-((S)-2-((S)-2-((tert-Butoxycarbonyl)amino)-3-methylbutanamido)propanamido)-2-(1H-tetrazol-5-yl)benzamido)-2-(4-(2-(2,4-diamino-5-fluoroquinazolin-6-yl)ethyl)benzamido) pentanoic acid (XT150)

Hydrolysis of ester XT149 (480 mg, 0.53 mmol) with LiOH (3 eq) was carried out according to general procedure XXE, to give acid XT150 (quant) as a light brown solid.

MS (ESI⁺) calc. for C₄₃H₅₃FN₁₃O₈⁺ [M+H]⁺ 898.4, found 898.7.

### (S)-2-(4-(2-(2,4-Diamino-5-fluoroquinazolin-6-yl)ethyl)benzamido)-5-(4-((S)-2-((S)-2-(6-(2,5-dioxo-2,5-dihydro-1H-pyrrol-1-yl)hexanamido)-3-methylbutanamido)propanamido)-2-(1H-tetrazol-5-yl)benzamido)pentanoic acid (XT151)

Following the procedure for XT114, carbamate XT150 (476 mg, 0.53 mmol) was deprotected and subsequently coupled with 6-maleimidohexanoic acid N-hydroxylsuccinimide ester (82 mg, 0.27 mmol). A small portion of crude product was purified by RP-HPLC (water×0.1% TFA/MeCN, gradient 20% to 50%) to afford XT151 (36 mg, 7%) as a white solid.

¹H NMR (400 MHz, DMSO-d₆) ppm=13.05-12.19 (m, 1H), 10.29 (s, 1H), 9.08-8.94 (m, 1H), 8.56 (br d, J=7.6 Hz, 1H), 8.48 (br s, 1H), 8.25 (br d, J=6.6 Hz, 2H), 7.94 (s, 1H), 7.90-7.71 (m, 5H), 7.71-7.57 (m, 2H), 7.30 (d, J=8.3 Hz, 2H), 7.18 (d, J=8.5 Hz, 1H), 7.00 (s, 2H), 6.55 (s, 1H), 4.43-4.30 (m, 2H), 4.16 (br d, J=1.0 Hz, 1H), 3.42-3.28 (m, 2H), 3.16 (br d, J=6.4 Hz, 2H), 2.97 (br d, J=2.8 Hz, 4H), 2.24-2.04 (m, 2H), 2.04-1.69 (m, 4H), 1.66-1.39 (m, 6H), 1.32 (d, J=7.1 Hz, 3H), 1.22-1.11 (m, 2H), 0.84 (dd, J=18.1, 6.8 Hz, 6H).

MS (ESI⁺) calc. for C₄₈H₅₆FN₁₄O₉⁺ [M+H]⁺ 991.4, found 991.5.

### Preparation of (S)-5-(4-amino-2-(1H-tetrazol-5-yl)benzamido)-2-(4-(2-(2,4-diamino-5-fluoroquinazolin-6-yl)ethyl)benzamido)pentanoic acid (XG2)

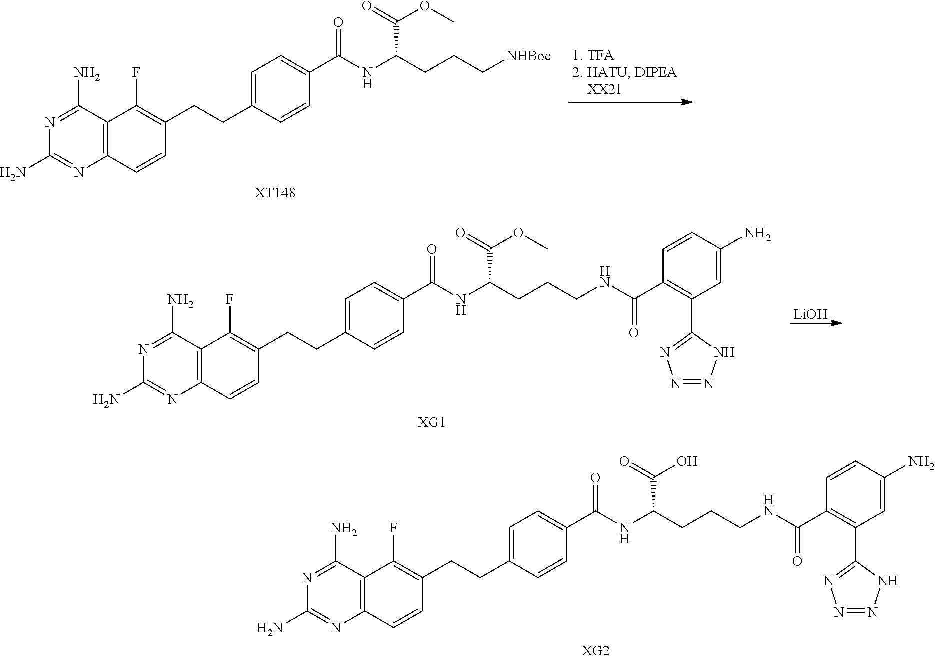

### Methyl (S)-5-(4-amino-2-(1H-tetrazol-5-yl)benzamido)-2-(4-(2-(2,4-diamino-5-fluoroquinazolin-6-yl)ethyl)benzamido)pentanoate (XG1)

Following general procedure XXD, carbamate XT148 (55 mg, 0.10 mmol) was reacted with a 4-amino-2-(2H-tetrazol-5-yl)benzoic acid solution XX21 (0.39 M, 182 μl, 0.07 mmol) in DMF to afford methyl ester XG1 (22 mg, 34%) as a light brown solid.

MS (ESI⁺) calc. for C₃₁H₃₃FN₁₁O₄⁺ [M+H]⁺ 642.3, found 642.5.

### (S)-5-(4-Amino-2-(1H-tetrazol-5-yl)benzamido)-2-(4-(2-(2,4-diamino-5-fluoroquinazolin-6-yl)ethyl)benzamido)pentanoic acid (XG2)

Saponification of methyl ester XG1 (22 mg, 0.034 mmol) was performed according to general procedure XXE. The crude solid was purified by preparative RP-HPLC (water×0.10% TFA/MeCN, gradient 20% to 50%). Combined product fractions were concentrated to remove MeCN followed by lyophilization to yield compound XG2 (11.3 mg, 44%) as a white solid.

¹H NMR (400 MHz, DMSO-d₆) ppm=12.58 (br d, J=1.9 Hz, 1H), 12.46 (br s, 1H), 9.05 (br s, 1H), 8.53 (d, J=7.6 Hz, 1H), 8.29 (br d, J=9.6 Hz, 1H), 8.18 (br s, 1H), 7.81 (d, J=8.3 Hz, 2H), 7.90 (br s, 1H), 7.69 (t, J=8.1 Hz, 1H), 7.45 (br d, J=7.4 Hz, 1H), 7.30 (d, J=8.3 Hz, 2H), 7.19 (d, J=8.5 Hz, 1H), 6.75-6.66 (m, 2H), 6.57 (br s, 1H), 5.85 (br s, 2H), 4.41-4.27 (m, 1H), 3.16-3.07 (m, 2H), 3.05-2.91 (m, 4H), 1.90-1.67 (m, 2H), 1.64-1.42 (m, 2H).

MS (ESI⁺) calc. for C₃₀H₃₁FN₁₁O₄⁺ [M+H]⁺ 628.3, found 628.4.

### Preparation of (S)-2-(4-(2-(2,4-diaminopteridin-6-yl)ethyl)-2-hydroxybenzamido)-5-(4-((S)-2-((S)-2-(6-(2,5-dioxo-2,5-dihydro-1H-pyrrol-1-yl)hexanamido)-3-methylbutanamido) propanamido)-2-(1H-tetrazol-5-yl)benzamido)pentanoic acid (XT160)

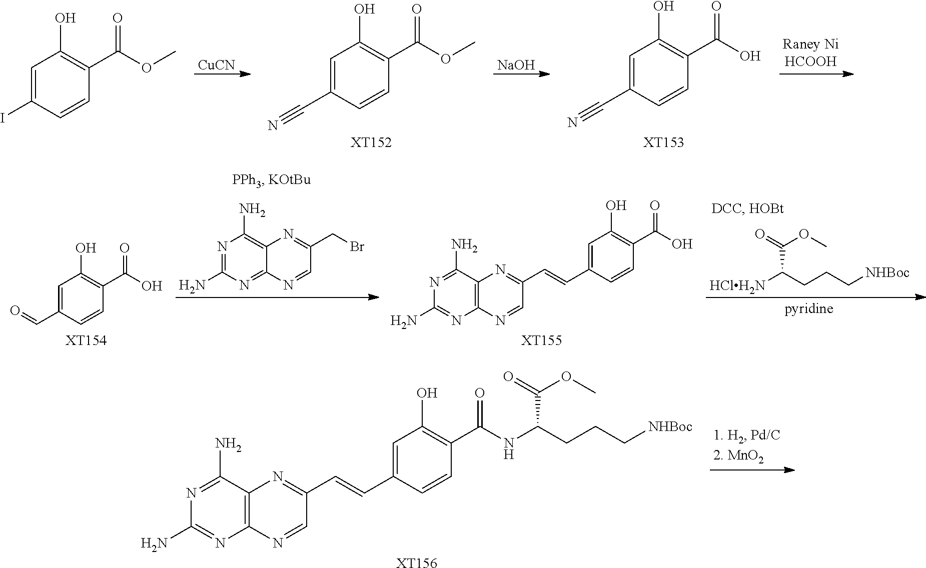

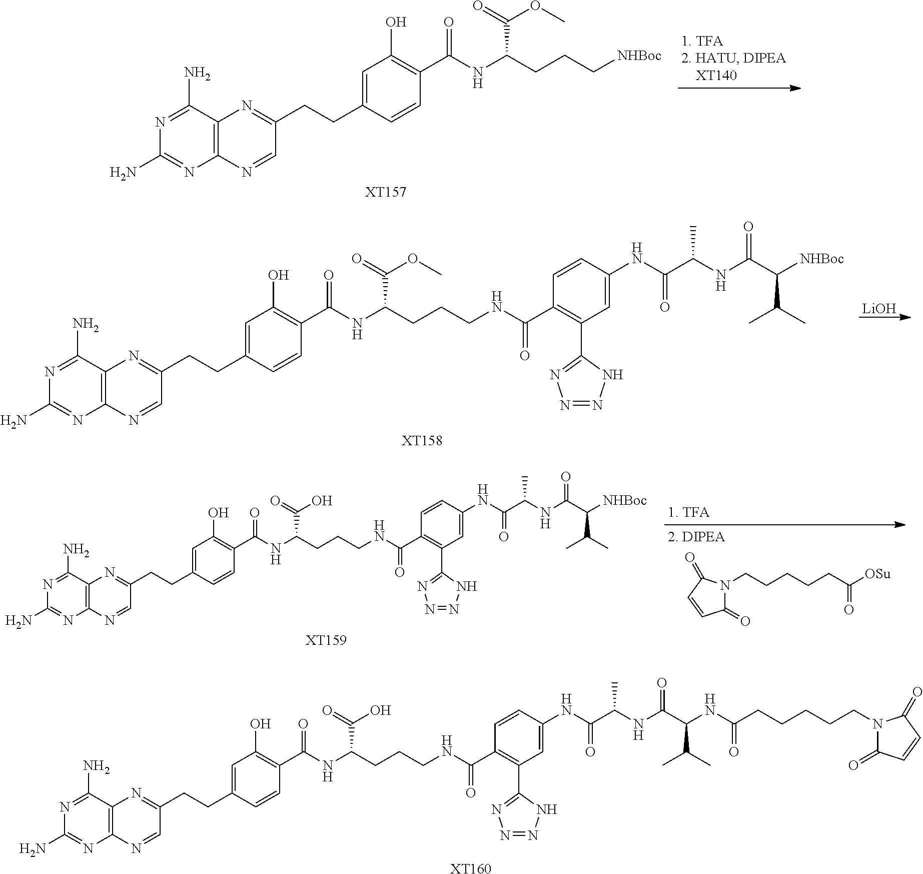

### Methyl 4-cyano-2-hydroxybenzoate (XT152)

A 20 mL reaction vessel was loaded with methyl 2-hydroxy-4-iodobenzoate (1.5 g, 5.4 mmol) and copper(I) cyanide (1.16 g, 12.9 mmol), purged with nitrogen followed by addition of DMF (12 mL). The resulting mixture was heated to 140° C. for 30 min. Afterwards, the mixture was cooled to RT, diluted with EtOAc (50 mL) and filtered over Celite®. The filtrate was diluted with water (35 mL) and EtOAc (50 mL) followed by addition of sat. NaHCO₃ (50 mL). The organic layer was separated and washed with water (50 mL) and brine and dried on Na₂SO₄, filtered and concentrated to yield methyl ester XT152 (900 mg, 94%) as a yellow solid.

¹H NMR (400 MHz, DMSO-d₆) ppm=10.89-10.62 (br s, 1H), 7.86 (d, J=8.1 Hz, 1H), 7.44 (s, 1H), 7.36 (d, J=8.1 Hz, 1H), 3.89 (s, 3H).

### 4-Cyano-2-hydroxybenzoic acid (XT153)

To an ice-bath cooled solution of methyl ester XT152 (880 mg, 5.0 mmol) in THF (10 mL) was added an aqueous NaOH solution (2 M, 7.45 mL, 14.9 mmol) and the resulting mixture was stirred for 72 h. Then, the mixture was acidified using aqueous KHSO₄ (0.5 M, 40 mL), extracted with Et₂O (2×) and the combined organic layer was washed with brine, dried on MgSO₄, filtered and concentrated to yield nitril XT153 (800 mg, 99%) as a white solid.

¹H NMR (400 MHz, DMSO-d₆) ppm=7.92 (d, J=8.1 Hz, 1H), 7.47 (d, J=1.5 Hz, 1H), 7.35 (dd, J=8.1, 1.6 Hz, 1H).

MS (ESI⁻) calc. for C₈H₄NO₃⁻ [M−H]⁻ 162.0, found 162.0.

### 4-Formyl-2-hydroxybenzoic acid (XT154)

To a suspension of nitril XT153 (800 mg, 4.9 mmol) in formic acid (10 mL) was added a Raney®-Nickel slurry (50% in H₂O, 1 mL, 4.90 mmol) and the resulting mixture was stirred at 100° C. for 3 h. Afterwards, the mixture was cooled to RT, diluted with MeOH (40 mL) and filtered over Celite®. The cake was rinsed once with MeOH and the filtrate was concentrated. The crude product was purified by flash chromatography (silica gel, DCM/MeOH, 0 to 20%) to yield aldehyde XT154 (795 mg, 98%) as a white solid.

¹H NMR (400 MHz, CDCl₃) ppm=10.78 (s, 1H), 8.17 (s, 1H), 7.83 (d, J=8.1 Hz, 1H), 6.99-6.97 (m, 1H), 6.86 (dd, J=8.1, 1.5 Hz, 1H).

MS (ESI⁻) calc. for C₈H₅O₄⁻ [M−H]⁻ 165.0, found 165.0.

### (E)-4-(2-(2,4-Diaminopteridin-6-yl)vinyl)-2-hydroxybenzoic acid (XT155)

Triphenylphosphane (635 mg, 2.421 mmol) was added to a solution of 6-(bromomethyl) pteridine-2,4-diamine (650 mg, 2.6 mmol) in DMA (35 mL) under a nitrogen-atmosphere at RT and the resulting solution was then heated at 65° C. for 60 min. Next, the mixture was cooled to 0° C., stirred for 15 min and KOtBu (1144 mg, 10.2 mmol) was added and stirred for 20 min. Then, aldehyde XT154 (423 mg, 2.6 mmol) was added and stirred for 4 h at RT. Afterwards, the mixture was cooled with an ice-bath, acetic acid (1.45 mL, 25.5 mmol) was added and stirred for 10 min, diluted with water (150 mL), cooled with an ice-bath and filtered. The obtained residue washed with MeCN (2×15 mL), toluene (2×15 mL) and Et₂O (2×20 mL) to yield carboxylic acid XT155 (550 mg, 66.6% yield) as a yellow solid.

MS (ESI⁺) calc. for C₁₅H₁₃N₆O₃⁺ [M+H]⁺ 325.1, found 325.2.

### Methyl (S,E)-5-((tert-butoxycarbonyl)amino)-2-(4-(2-(2,4-diaminopteridin-6-yl)vinyl)-2-hydroxybenzamido)pentanoate (XT156)

To a suspension of carboxylic acid XT155 (450 mg, 1.39 mmol) and methyl (S)-2-amino-5-((tert-butoxycarbonyl)amino)pentanoate hydrochloride (589 mg, 2.08 mmol) in pyridine (45 mL) were added DCC (344 mg, 1.67 mmol) and HOBt (255 mg, 1.67 mmol) and the resulting mixture was stirred for 4 h at 80° C. Then, the mixture was cooled to RT, concentrated, suspended in water (25 mL) and stirred for 15 min. The suspension was filtered, the residue washed with Et₂O and dried on air to yield alkene XT156 (1040 mg) as an orange solid and was used without further purification in the next step.

¹H NMR (400 MHz, DMSO-d₆) ppm=12.31 (s, 1H), 9.02 (d, J=7.3 Hz, 1H), 8.90 (br s, 1H), 8.24-8.05 (m, 2H), 8.00 (d, J=8.3 Hz, 1H), 7.93-7.86 (m, 1H), 7.54-7.47 (m, 1H), 7.30-7.21 (m, 1H), 7.16 (s, 1H), 7.12-6.97 (br s, 2H), 6.88-6.82 (m, 1H), 4.57-4.46 (m, 1H), 3.68 (s, 3H), 3.00-2.89 (m, 2H), 1.92-1.71 (m, 2H), 1.57-1.43 (m, 2H), 1.37 (s, 9H).

MS (ESI⁺) calc. for C₂₆H₃₃N₈O₆⁺ [M+H]⁺ 553.3, found 553.6.

### Methyl (S)-5-((tert-butoxycarbonyl)amino)-2-(4-(2-(2,4-diaminopteridin-6-yl)ethyl)-2-hydroxybenzamido)pentanoate (XT157)

To a nitrogen purged solution of alkene XT156 (774 mg, 1.4 mmol) in AcOH (40 mL) was added Pd/C (750 mg, 7.1 mmol) and the resulting mixture was then purged with hydrogen (3×) and stirred under a hydrogen atmosphere for 24 h. Then, the reaction mixture was filtered over Celite® and the cake was rinsed with THF (40 mL). Next, manganese dioxide (365 mg, 4.2 mmol) was added to the filtrate and stirred for 5 h, followed by filtration over Celite®, additional rinsing of the cake with THF and then concentration of the filtrate. The crude product was purified by flash chromatography (silica gel, DCM/MeOH, 0 to 15%), to yield carbamate XT157 (380 mg, 49% over 3 steps).

¹H NMR (400 MHz, DMSO-d₆) ppm=12.41-11.89 (m, 1H), 8.97-8.88 (m, 1H), 8.56 (s, 1H), 7.83 (d, J=8.5 Hz, 1H), 7.65-7.51 (m, 2H), 6.81 (s, 3H), 6.63-6.44 (m, 2H), 4.52-4.42 (m, 1H), 3.65 (s, 3H), 3.10 (br d, J=2.4 Hz, 4H), 2.98-2.86 (m, 2H), 1.87-1.67 (m, 2H), 1.53-1.41 (m, 2H), 1.36 (s, 9H).

MS (ESI⁺) calc. for C₂₆H₃₅N₈O₆⁺ [M+H]⁺ 555.3, found 555.4.

### Methyl (S)-5-(4-((S)-2-((S)-2-((tert-butoxycarbonyl)amino)-3-methylbutanamido) propanamido)-2-(JH-tetrazol-5-yl)benzamido)-2-(4-(2-(2,4-diaminopteridin-6-yl)ethyl)-2-hydroxybenzamido)pentanoate (XT158)

Following general procedure XXD, carbamate XT157 (240 mg, 0.43 mmol) was reacted with XT140 (165 mg, 0.35 mmol) in DMF to afford methyl ester XT158 (quant) as a light brown solid.

MS (ESI⁺) calc. for C₄₂H₅₄N₁₅O₉⁺ [M+H]⁺ 912.4, found 912.9.

### (S)-5-(4-((S)-2-((S)-2-((tert-Butoxycarbonyl)amino)-3-methylbutanamido)propanamido)-2-(1H-tetrazol-5-yl)benzamido)-2-(4-(2-(2,4-diaminopteridin-6-yl)ethyl)-2-hydroxybenzamido) pentanoic acid (XT159)

The hydrolysis of ester XT158 (319 mg, 0.35 mmol) with LiOH (3 eq) was carried out according to general procedure XXE, to give acid XT159 (quant) as a light brown solid.

MS (ESI⁺) calc. for C₄₆H₅₂N₁₅O₉⁺ [M+H]⁺ 898.4, found 898.9.

### (S)-2-(4-(2-(2,4-Diaminopteridin-6-yl)ethyl)-2-hydroxybenzamido)-5-(4-((S)-2-((S)-2-(6-(2,5-dioxo-2,5-dihydro-1H-pyrrol-1-yl)hexanamido)-3-methylbutanamido)propanamido)-2-(1H-tetrazol-5-yl)benzamido)pentanoic acid (XT160)

Following the procedure of XT114, carbamate XT159 (386 mg, 0.43 mmol) was deprotected and subsequently coupled with 6-maleimidohexanoic acid N-hydroxylsuccinimide ester (80 mg, 0.26 mmol). A small portion of crude product was purified by RP-HPLC (water×0.1% TFA/MeCN, gradient 20% to 50%) to afford XT160 (30 mg, 12%) as a white solid.

¹H NMR (400 MHz, DMSO-d₆) ppm=13.25-12.45 (m, 1H), 12.25 (br d, J=5.5 Hz, 1H), 10.28 (s, 1H), 9.10-8.72 (m, 3H), 8.72 (s, 1H), 8.50-7.30 (br s, 2H), 8.45 (br s, 1H), 8.23 (br d, J=6.5 Hz, 1H), 7.94 (s, 1H), 7.88 (br d, J=8.0 Hz, 1H), 7.83-7.73 (m, 2H), 7.63 (br d, J=8.4 Hz, 1H), 7.00 (s, 2H), 6.81 (s, 2H), 6.61-6.48 (m, 1H), 4.47-4.34 (m, 2H), 4.19-4.12 (m, 1H), 3.40-3.30 (2H, m), 3.23-3.06 (m, 6H), 2.22-2.04 (m, 2H), 2.02-1.73 (m, 3H), 1.67-1.39 (m, 6H), 1.39-1.29 (m, 3H), 1.29-1.11 (m, 2H), 0.93-0.75 (m, 6H).

MS (ESI⁺) calc. for C₄₆H₅₅N₁₆O₁₀⁺ [M+H]⁺ 991.4, found 991.8.

### Preparation of (S)-5-(4-amino-2-(1H-tetrazol-5-yl)benzamido)-2-(4-(2-(2,4-diaminopteridin-6-yl)ethyl)-2-hydroxybenzamido)pentanoic acid (XT162)

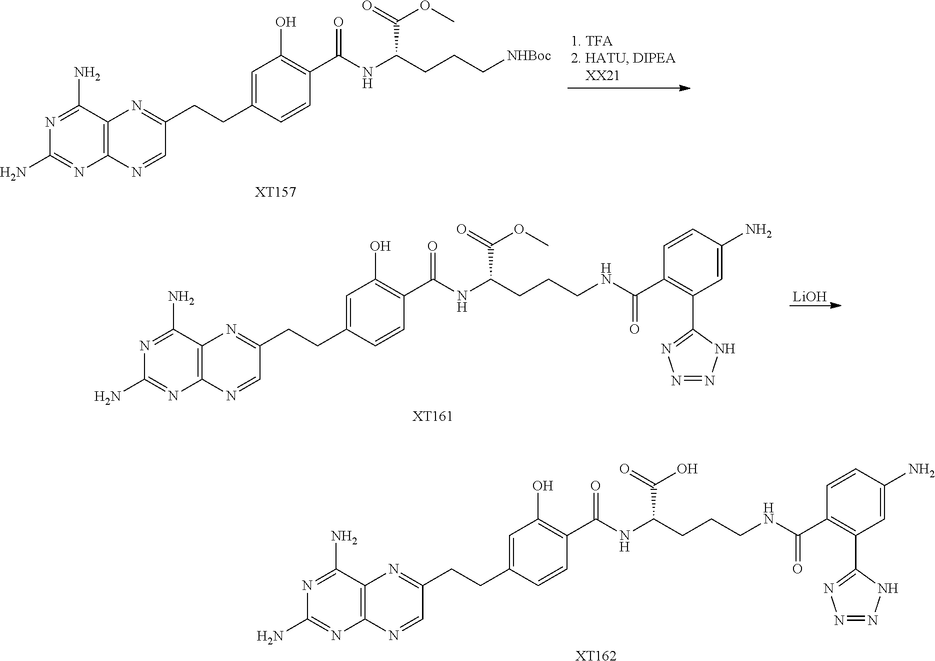

### Methyl (S)-5-(4-amino-2-(1H-tetrazol-5-yl)benzamido)-2-(4-(2-(2,4-diaminopteridin-6-yl) ethyl)-2-hydroxybenzamido)pentanoate (XT161)

Following general procedure XXD, carbamate XT157 (100 mg, 0.18 mmol) was reacted with a solution of 4-amino-2-(2H-tetrazol-5-yl)benzoic acid XX21 (0.39 M, 375 μL, 0.14 mmol) in DMF to afford methyl ester XT161 (quant) as a light brown solid.

MS (ESI⁺) calc. for C₂₉H₃₂N₁₃O₅⁺ [M+H]⁺ 642.3, found 642.4.

### (S)-5-(4-Amino-2-(1H-tetrazol-5-yl)benzamido)-2-(4-(2-(2,4-diaminopteridin-6-yl)ethyl)-2-hydroxybenzamido)pentanoic acid (XT162)

Saponification of methyl ester XT161 (20 mg, 0.03 mmol) was performed according to general procedure XXE. The crude solid was purified by preparative RP-HPLC (water×0.1% TFA/MeCN, gradient 5% to 35%). Combined product fractions were concentrated to remove MeCN followed by lyophilization to yield compound XT162 (20 mg, 23%) as a white solid.

¹H NMR (400 MHz, DMSO-d₆) ppm=12.19 (br s, 1H), 8.80 (br d, J=7.5 Hz, 1H), 8.59 (s, 1H), 8.46-8.20 (m, 2H), 8.14 (s, 1H), 7.79 (d, J=8.5 Hz, 1H), 7.37 (d, J=9.1 Hz, 1H), 6.82-6.68 (m, 2H), 6.68-6.57 (m, 2H), 6.54-6.44 (m, 1H), 6.12-5.57 (m, 1H), 5.74 (br s, 2H), 4.38-4.26 (m, 1H), 3.13-2.99 (m, 6H), 1.88-1.62 (m, 2H), 1.55-1.36 (m, 2H), 1.25-1.13 (m, 2H).

MS (ESI⁺) calc. for C₂₈H₃₀N₁₃O₅⁺ [M+H]⁺ 628.3, found 628.4.

Further antifolate compounds prepared via analogous syntheses are:

| Compound | Structure |
|---|---|
| AFD-001 | 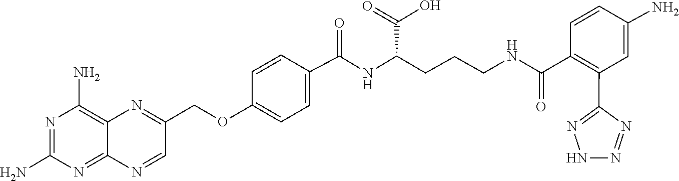 [SMILES: Nc1ccc(C(=O)NCCC[C@H](NC(=O)c2ccc(OCc3cnc4nc(N)nc(N)c4n3)cc2)C(=O)O)c(-c2nn[nH]n2)c1] |
| AFD-002 | 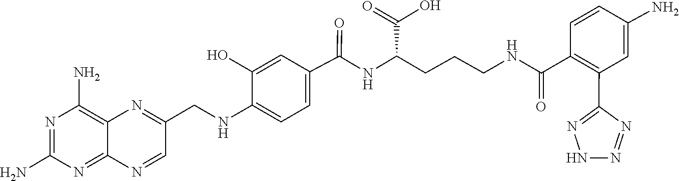 [SMILES: Nc1ccc(C(=O)NCCC[C@H](NC(=O)c2ccc(NCc3cnc4nc(N)nc(N)c4n3)c(O)c2)C(=O)O)c(-c2nn[nH]n2)c1] |
| AFD-003 | 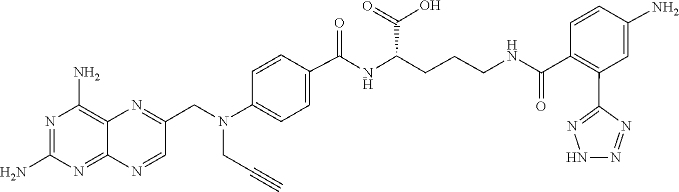 [SMILES: C#CCN(Cc1cnc2nc(N)nc(N)c2n1)c1ccc(C(=O)N[C@@H](CCCNC(=O)c2ccc(N)cc2-c2nn[nH]n2)C(=O)O)cc1] |
| AFD-004 | 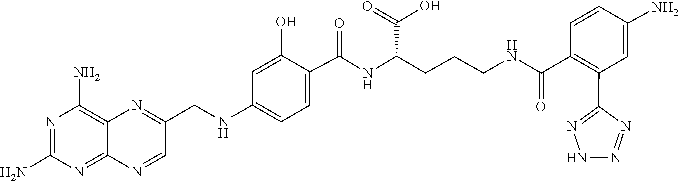 [SMILES: Nc1ccc(C(=O)NCCC[C@H](NC(=O)c2ccc(NCc3cnc4nc(N)nc(N)c4n3)cc2O)C(=O)O)c(-c2nn[nH]n2)c1] |
| AFD-005 | 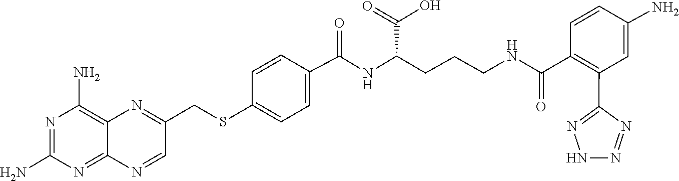 [SMILES: Nc1ccc(C(=O)NCCC[C@H](NC(=O)c2ccc(SCc3cnc4nc(N)nc(N)c4n3)cc2)C(=O)O)c(-c2nn[nH]n2)c1] |
| AFD-006 | 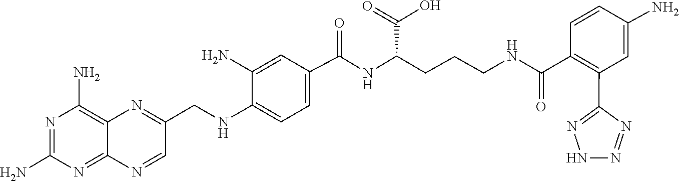 [SMILES: Nc1ccc(C(=O)NCCC[C@H](NC(=O)c2ccc(NCc3cnc4nc(N)nc(N)c4n3)c(N)c2)C(=O)O)c(-c2nn[nH]n2)c1] |
| AFD-007 | 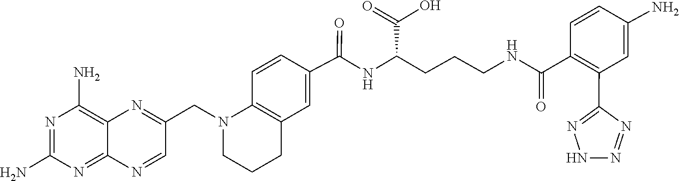 [SMILES: Nc1ccc(C(=O)NCCC[C@H](NC(=O)c2ccc3c(c2)CCCN3Cc2cnc3nc(N)nc(N)c3n2)C(=O)O)c(-c2nn[nH]n2)c1] |
| AFD-008 | 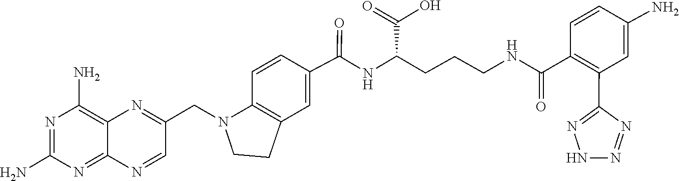 [SMILES: Nc1ccc(C(=O)NCCC[C@H](NC(=O)c2ccc3c(c2)CCN3Cc2cnc3nc(N)nc(N)c3n2)C(=O)O)c(-c2nn[nH]n2)c1] |
| AFD-009 | 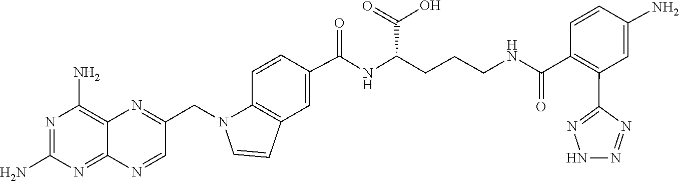 [SMILES: Nc1ccc(C(=O)NCCC[C@H](NC(=O)c2ccc3c(ccn3Cc3cnc4nc(N)nc(N)c4n3)c2)C(=O)O)c(-c2nn[nH]n2)c1] |
| AFD-010 | 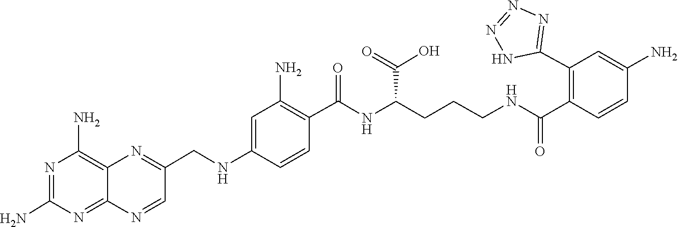 [SMILES: Nc1ccc(C(=O)NCCC[C@H](NC(=O)c2ccc(NCc3cnc4nc(N)nc(N)c4n3)cc2N)C(=O)O)c(-c2nnn[nH]2)c1] |

### Use of the Compounds

### Cancer Cell Lines

Human tumor cell lines SK-BR-3, SW-620, A-549, BT-474, Jurkat Clone E6-1, AU-565, and SK-OV-3 were obtained from American Type Culture Collection (Rockville, MD, USA). FaDu, BxPC-3, HeLa, MV4-11, and RAJI cell lines were obtained from Deutsche Sammlung von Mikroorganismen und Zellkulturen (Leipzig, Germany). Jurkat NucLight Red cells were obtained from Essen BioScience Inc. (Ann Arbor, MI, USA). SK-BR-3 and SK-OV-3 cells were cultured in McCoys 5A medium (Lonza; Walkersville, MD, USA) supplemented with 10% v/w Fetal Bovine Serum (FBS), Heat-inactivated (HI) (Gibco-Life Technologies; Carlsbad, CA) and 80 U/mL Pen/Strep (Gibco-Life Technologies), at 37° C. in a humidified incubator under 5% CO₂ atmosphere. SW-620 cells were similarly maintained in RPMI 1640 medium (Lonza), containing 10% v/w FBS HI and 80 U/mL Pen/Strep. A-549 cells were similarly maintained in F-12K Nutrient Mixture (1×) (Gibco-Life Technologies) media supplemented with 80 U/mL Pen/Strep and 5% v/w FBS, which was Qualified (Q) (Gibco-Life Technologies). BT-474 and AU-565 cells were similarly maintained in RPMI 1640 medium (Lonza), containing 10% v/w FBS, which was Qualified (Q) (Gibco-Life Technologies), and 80 U/mL Pen/Strep. Jurkat Clone E6-1, BxPC-3, HeLa, MV4-11, and RAJI cells were similarly maintained in RPMI 1640 medium (Lonza), containing 10% v/w FBS HI and 80 U/mL Pen/Strep. Jurkat NucLight Red cells were similarly maintained in RPMI 1640 medium (Lonza), containing 10% v/w FBS HI, 80 U/mL Pen/Strep and 0.5 μg/mL Puromycin (Gibco-Life Technologies). FaDu cells were cultured in EMEM medium (Lonza) supplemented with 10% v/w FBS HI (Gibco-Life Technologies), 1× GlutaMAX (Gibco-Life Technologies), and 80 U/mL Pen/Strep (Gibco-Life Technologies), at 37° C. in a humidified incubator under 5% CO₂ atmosphere.

### In Vitro Cell Viability Assays

Cells in complete growth medium were plated in 96-well plates (90 μL/well) and incubated at 37° C., 5% CO₂ at the following cell densities: 6500 SK-BR-3, 4000 SW-620, 2500 A-549, 10000 BT-474, 3000 Jurkat Clone E6-1, 5000 AU-565, 1250 FaDu, 1250 BxPC-3, 1000 HeLa, 7500 MV4-11, 1000 RAJI, 3000 Jurkat NucLight Red, and 4000 SK-OV-3 cells per well. After an overnight incubation, 10 serial log 10 dilutions of the free drugs were made in DMSO (Sigma-Aldrich). These free drug dose-response curves were 10-times further diluted in complete growth medium. For the ADCs according to the invention 10 serial log 10 dilutions were made in complete growth medium. 10 μL of a composition comprising a free drug or an ADC according to the invention was added to the assay plate (final DMSO content for free drugs was 1%). A final DMSO concentration of 0.1% was used when a cell line showed a decrease in viability by 1% DMSO. Cell viability was assessed after 6 days using a luminescent assay kit (CellTiter-Glo™ (CTG), Promega Corporation) according to the manufacturer's instructions. Percentage cell survival was calculated by dividing the measured luminescence for each free drug or ADC according to the concentration with the average mean of untreated cells (1% or 0.1% DMSO control (for free drugs) or 100% complete growth medium (for ADCs)) multiplied by 100.

Curves were fitted by non-linear regression using the sigmoidal dose-response equation with variable slope (four parameters) using curve-fitting software (GraphPad Prism, version 9.4.0 for Windows, GraphPad, San Diego, CA or Electronic Laboratory Notebook (ELN) add-in BioAssay, version 19.1.0.0218, Perkin Elmer, Waltham, MA). Relative IC₅₀ values were calculated as the concentration that gives a response halfway between bottom and top of the curve, when using a 4-parameter logistic fit. Data were reported as mean IC₅₀ value of at least one experiment performed in duplicate.

Experiments with Free Antifolate Drugs

### Results

TABLE 1a
IC₅₀ values of free antifolate drugs

| | IC₅₀ (nM)* | | | | | |
|---|---|---|---|---|---|---|
| | SK-BR-3 | SW-620 | A-549 | BT-474 | AU-565 | SK-OV-3 |
| Compound | cells | cells | cells | cells | cells | cells |
| Talotrexin | 1.83 | 1.74 | 1.70 | — | — | — |
| Comparative | 0.92 | 1.41 | 1.97 | — | — | — |
| compound 1 | | | | | | |
| Mixture of XT9 | 0.89 | 1.11 | 1.72 | — | — | — |
| and XT10 | | | | | | |
| XT10 | 1.9 | 2.1 | 2.0 | — | — | — |
| XT9 | 17 | 10 | 20 | — | — | — |
| XT21 | 2.1 | 2.9 | 2.3 | — | — | — |
| XT20 | 25 | 12 | 19 | — | — | — |
| XT25 | 1.0 | 1.1 | 2.3 | — | — | — |
| XT24 | 18 | 7.5 | 27 | — | — | — |
| XT35 | 3.1 | 2.5 | 16 | — | — | — |
| XX12 | 17 | 7.5 | 42 | — | — | — |
| XX7 | 6.5 | 2.8 | 29 | — | — | — |
| XX5 | 16 | 7.2 | 76 | — | — | — |
| XJ4 | 3.4 | — | ~10^{$} | 8.1 | — | — |
| XX35 | 1.3 | — | ~2.5^{$} | 4.3 | — | — |
| XX37 | 2.4 | — | 4.0 | 7.5 | — | — |
| XX47 | ~14^{#} | — | — | ~100^{#} | — | >100 |
| XR22 | 2.2 | — | ~2.5^{$} | — | 0.9 | — |
| XS14 | 1.6 | — | ~8.7^{$} | — | 1.9 | — |

*Talotrexin, XJ4, XX35, XX37, XX47, XR22, and XS14 IC₅₀s in Table 1a were determined with GraphPad Prism software. The IC₅₀s for the other compounds were determined with BioAssay software;
^{#}No full dose-response curve, bottom is missing;
^{$}Steep hillslope;
— Not tested.

TABLE 1b
IC₅₀ values of free antifolate drugs

| | IC₅₀ (nM)* | | | | | |
|---|---|---|---|---|---|---|
| | | | | | | Jurkat |
| Com- | SK-BR-3 | SW-620 | A-549 | BT-474 | AU-565 | NucLight |
| pound | cells | cells | cells | cells | cells | Red cells |
| XX35 | 1.03 | — | — | — | 0.62 | 1.4 |
| XT128 | 0.57 | — | — | — | 0.34 | 0.61 |
| XT110 | 0.41 | — | — | — | 0.36 | 0.75 |
| XT132 | 2.15 | — | — | — | 0.97 | 1.19 |
| AFD-001 | 25^{#} | — | — | — | 9.18 | 4.92 |
| AFD-002 | 20^{#} | — | — | — | 14 | 15^{#} |
| XT126 | 2.91 | — | — | — | 2.37 | 5.01 |
| AFD-003 | 2.09 | — | — | — | 0.98 | 1.4 |

*The IC₅₀s for the compounds in Table 1b were determined with BioAssay software;
^{#}No full dose-response curve, bottom is missing;
— Not tested.

TABLE 1c
IC₅₀ values of free antifolate drugs

| | IC₅₀ (nM)* | | | | | |
|---|---|---|---|---|---|---|
| | | | | | | Jurkat |
| | SK-BR-3 | SW-620 | A-549 | BT-474 | AU-565 | NucLight |
| Compound | cells | cells | cells | cells | cells | Red cells |
| XT128 | 0.78 | — | — | — | 0.36 | 0.71 |
| XT110 | 0.54 | — | — | — | 0.44 | 0.92 |
| AFD-004 | 0.67 | — | — | — | 0.37 | 0.68 |
| AFD-005 | ~22* | — | — | — | ~8* | 5.6 |
| XT123 | 0.44 | — | — | — | 0.35 | 0.6 |
| AFD-006 | 5.15 | — | — | — | 2.8 | 5.98 |
| AFD-007 | 1.06 | — | — | — | 0.68 | 2.01 |
| XC5 | ~100^{#} | — | — | — | ~28^{#} | ~30^{#} |
| XC9 | 3.77 | — | — | — | 1.95 | 2.41 |
| AFD-008 | 1.5 | — | — | — | 0.76 | 0.87 |
| AFD-009 | 2.82 | — | — | — | 1.15 | 1.96 |
| AFD-010 | 2.4 | — | — | — | 1.17 | 2.31 |

*The IC₅₀s for the compounds in Table 1c were determined with BioAssay software;
^{#}No full dose-response curve, bottom is missing;
*No full dose-response curve, bottom is missing and lingering/dragging hill slope;
— Not tested.

TABLE 1d
IC₅₀ values of free antifolate drugs

| | IC₅₀ (nM)* | | | | |
|---|---|---|---|---|---|
| | SK-BR-3 | BT-474 | Jurkat | AU-565 | |
| Compound | cells | cells | clone E6-1 | cells | FaDu cells |
| XT123 | 0.63 | 0.17 | — | 0.53 | 0.19 |
| XT162 | 0.86 | — | 0.78 | 0.92 | 0.44 |
| XG2 | 0.90 | — | — | 0.86 | 0.31 |

| | IC₅₀ (nM)* | | | | |
|---|---|---|---|---|---|
| | | | | | Jurkat |
| | BxPC-3 | HeLa | MV4-11 | | NucLight |
| Compound | cells | cells | cells | RAJI cells | Red cells |
| XT123 | 0.71 | 0.11 | 0.62 | 0.60 | 0.79 |
| XT162 | 0.78 | — | — | — | 0.66 |
| XG2 | 0.92 | — | — | — | 0.87 |

*The IC₅₀s for the compounds in Table 1d were determined with BioAssay software;
— Not tested.

### Comparative Compound 1

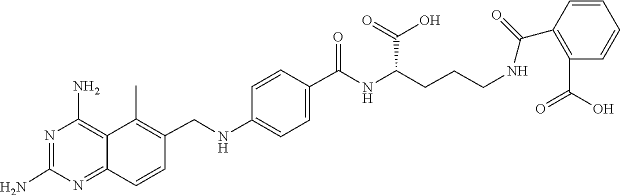 [SMILES: Cc1c(CNc2ccc(C(=O)N[C@@H](CCCNC(=O)c3ccccc3C(=O)O)C(=O)O)cc2)ccc2nc(N)nc(N)c12]

Comparative compound 1 was synthesised according to the procedure in Rosowsky et al, J. Med. Chem. 1998, 41, 5310-5319.

Compounds XJ4, XX5, XX7, XX12 and XT35, not having the COOH-substituent on the phenyl-ring, are less active than talotrexin and the other antifolate compounds that do have the COOH-substituent. Replacing the COOH-substituent for SO₃H or tetrazole had little effect on the antifolate activity, whereas replacing the COOH-substituent for CN seemed to decrease the activity in SK-BR-3 cells. Combining a tetrazole ring as in XX35 with a replacement of the pteridine-2,4-diamine moiety with a 5-chloroquinazoline-2,4-diamine moiety as in XT110 strongly increased the antifolate activity. Replacing the amide bond (Q) for triazole, an amide bond bioistostere, had little effect on the antifolate activity. The introduction of an NH₂- or OH-substituent on the V-moiety phenyl-ring in the para position yields compounds with an antifolate activity comparable to that of talotrexin. The antifolate activity of compounds with the NH₂-substituent in the meta position is approximately 10-fold lower when compared to the corresponding para-compounds and talotrexin (not having the NH₂-substituent).

### Experiments with Antibody-Drug Conjugates

### Preparation of the DAR2 Site-Specific Conjugate

To a solution of HC41C engineered trastuzumab (10 mg/mL, 100 mM histidine, pH 5) was added 2-(diphenylphosphino)benzenesulfonic acid (diPPBS) (16-32 molar equivalents per molar equivalent of the engineered antibody, 10 mM in water (MilliQ®)) and the resulting mixture was incubated at RT for 16-24 h. The excess diPPBS was removed by a centrifugal concentrator (Vivaspin filter, 30 kDa cut-off, PES) using 4.2 mM histidine, 50 mM trehalose, pH 6 or by carbon filtration. DMA was added followed by a solution of linker-drug (10 mM in DMA, 3.5 eq). The final concentration of DMA was 10%. The resulting mixture was incubated at RT in the absence of light for 3 h. In order to remove the excess of linker-drug, activated charcoal was added and the mixture was incubated at RT for at least 0.5 h. The charcoal was removed using a 0.2 μm PES or PVDF filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.2 m PVDF filter.

### Preparation of the DAR2 and DAR4 Wild-Type Conjugates

To a solution of trastuzumab (12 mg/mL in 4.2 mM histidine, 50 mM trehalose, pH 6) EDTA (25 mM in water, 4% v/v) and TRIS (1 M in water, pH 8, 1% v/v) were added. TCEP (10 mM in water, 1.1 eq for DAR2 and 2.2 eq for DAR4) was added and the resulting mixture was incubated at RT overnight. The reactants were removed by a centrifugal concentrator (Vivaspin filter, 30 kDa cut-off, PES) using 4.2 mM histidine, 50 mM trehalose, pH 6. DMA was added followed by a solution of linker-drug (10 mM in DMA, 4 eq for DAR2 and 8 eq for DAR4). The final concentration of DMA was 10%. The resulting mixture was incubated at RT in the absence of light for 3 h. In order to remove the excess of linker-drug, activated charcoal was added and the mixture was incubated at RT for 1 h. The coal was removed using a 0.2 m PES or PVDF filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.2 m PVDF filter.

### Preparation of the DAR8 Wild-Type Conjugate

To a solution of trastuzumab, sacituzumab, or non-binding control antibody rituximab (12 mg/mL in 4.2 mM histidine, 50 mM trehalose, pH 6) EDTA (25 mM in water, 4% v/v) and TRIS (1 M in water, pH 8, 2% v/v) were added. TCEP (10 mM in water, 30 eq) was added and the resulting mixture was incubated at RT overnight. The reactants were removed by a centrifugal concentrator (Vivaspin filter, 30 kDa cut-off, PES) using 4.2 mM histidine, 50 mM trehalose, pH 6. DMA was added followed by a solution of linker-drug (10 mM in DMA, 14 eq). The final concentration of DMA was 10%. The resulting mixture was incubated at RT in the absence of light for 3 h or overnight. In order to remove the excess of linker-drug, activated charcoal was added and the mixture was incubated at RT for 1 h. The coal was removed using a 0.2 m PES or PVDF filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.2 m PVDF filter.

### Preparation of the DAR10 Site-Specific and Wild-Type Conjugate

To a solution of anti-5T4 antibody 825a (12 mg/mL in 4.2 mM histidine, 50 mM trehalose, pH 6) EDTA (25 mM in water, 4% v/v) and TRIS (1 M in water, pH 8, 2% v/v) were added. TCEP (10 mM in water, more than 30 eq) was added and the resulting mixture was incubated at RT overnight. As a result of the excess of TCEP both wild-type and engineered cysteines were reduced. The reactants were removed by a centrifugal concentrator (Vivaspin filter, 30 kDa cut-off, PES) using 4.2 mM histidine, 50 mM trehalose, pH 6. DMA was added followed by a solution of linker-drug (10 mM in DMA, 14 eq). The final concentration of DMA was 10%. The resulting mixture was incubated at RT in the absence of light for 3 h or overnight. In order to remove the excess of linker-drug, activated charcoal was added and the mixture was incubated at RT for 1 h. The coal was removed using a 0.2 m PES or PVDF filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.2 m PVDF filter.

TABLE 2
Characteristics of antifolate ADCs

| | | | |
|---|---|---|---|
| | | | Retention |
| | | | Time |
| Compound | % HMW | DAR | (HIC) (min) |

Trastuzumab conjugate

| | | | |
|---|---|---|---|
| DAR0 | — | — | 6.6 |
| trastuzumab antibody | | | |
| DAR2 - XT17 | 0.3 | 2 | 6.8 |
| site-specific conjugation (HC-41C) | | | |
| DAR2 - XT17 | 0.5 | 2 | 6.9 |
| wild-type conjugation | | | |
| DAR4 - XT17 | 0.6 | 4 | 6.9 |
| wild-type conjugation | | | |
| DAR8 - XT17 | 0.6 | 8 | 7.6 |
| wild-type conjugation | | | |
| DAR8 - XS23 | 2.3 | 8 | 8.3 |
| wild-type conjugation | | | |
| DAR8 - XT16 | 2.0 | 8 | 7.64 |
| wild-type conjugation | | | |
| DAR8 - XT31/XT32 | 2.1 | 8 | 7.21 |
| wild-type conjugation | | | |
| DAR8 - XT46 | 2.0 | 8 | 9.28 |
| wild-type conjugation | | | |
| DAR8 - XT41 | 3.8 | 8 | 8.43 |
| wild-type conjugation | | | |
| DAR8 - XR4 | 4.6 | 8 | 10.13 |
| wild-type conjugation | | | |
| DAR8 - XT54 | 2.5 | 8 | 8.15 |
| wild-type conjugation | | | |
| DAR8 - XR8-p/XR8-m | 4.1 | 8 | 9.08 |
| wild-type conjugation | | | |
| DAR8 - XX19 | 2.7 | 8 | 8.63 |
| wild-type conjugation | | | |
| DAR8 - XX28 | 2.8 | 8 | 9.68 |
| wild-type conjugation | | | |
| DAR8 - XS6 | 4.0 | 8 | 9.48 |
| wild-type conjugation | | | |
| DAR8 - XS12 | 3.8 | 8 | 7.84 |
| wild-type conjugation | | | |
| DAR8 - XT89 | 1.7 | 8 | 8.32 |
| wild-type conjugation | | | |
| DAR8 - XR12 | 3.8 | 8 | 10.82 |
| wild-type conjugation | | | |
| DAR8 - XR16 | 2.0 | 8 | 8.57 |
| wild-type conjugation | | | |
| DAR8 - XX23 | 2.6 | 8 | 8.27 |
| wild-type conjugation | | | |
| DAR8 - XT63 | 3.0 | 8 | 8.34 |
| wild-type conjugation | | | |
| DAR8 - XT723 | 3.0 | 8 | 8.67 |
| wild-type conjugation | | | |
| DAR8 - XT94 | 2.3 | 8 | 8.79 |
| wild-type conjugation | | | |
| DAR8 - XT97 | 1.5 | 8 | 8.88 |
| wild-type conjugation | | | |
| DAR8 - XJ9 | 2.0 | 8 | 8.19 |
| wild-type conjugation | | | |
| DAR8 - XT100 | 1.9 | 8 | 11.8 |
| wild-type conjugation | | | |
| DAR8 - XJ13 | 2.5 | 8 | 8.86 |
| wild-type conjugation | | | |
| DAR8 - XJ21 | 0.7 | 8 | 7.64 |
| wild-type conjugation | | | |
| DAR8 - XT114 | 1.9 | 8 | 7.98 |
| wild-type conjugation | | | |
| DAR8 - XT123 | 0.89 | 8 | 7.97 |
| wild-type conjugation | | | |
| DAR8 - XT162 | 0.96 | 8 | 9.64 |
| wild-type conjugation | | | |
| DAR8 - XG2 | 0.88 | 8 | 8.53 |
| wild-type conjugation | | | |

Sacituzumab conjugate

| | | | |
|---|---|---|---|
| DAR8 - XT123 | 1.0-2.0 | 8 | 10.25 |
| wild-type conjugation | | | |
| DAR8 - XT162 | 1.4-2.2 | 8 | 10.84 |
| wild-type conjugation | | | |
| DAR8 - XG2 | 0.8-2.1 | 8 | 10.19 |
| wild-type conjugation | | | |

825a conjugate

| | | | |
|---|---|---|---|
| DAR10 - XT17 | 6.3 | 10 | 8.03 |
| wild-type and site-specific conjugation | | | |
| DAR10 - XR16 | 3.2 | 10 | 8.75 |
| wild-type and site-specific conjugation | | | |
| DAR10 - XX23 | 5.0 | 10 | 8.59 |
| wild-type and site-specific conjugation | | | |

Control conjugate

| | | | |
|---|---|---|---|
| DAR8 - XT17 | 1.0 | 8 | 8.3 |
| non-binding control conjugate | | | |

### Results

TABLE 3a
IC₅₀ values of antifolate ADCs

| | SK-BR-3 cells | | SW-620 cells | | BT-474 cells | | AU-565 cells | | SK-OV-3 cells | |
|---|---|---|---|---|---|---|---|---|---|---|
| | IC₅₀ | Efficacy | IC₅₀ | Efficacy | IC₅₀ | Efficacy | IC₅₀ | Efficacy | IC₅₀ | Efficacy |
| Compound | (μg/mL) | (%) | (μg/mL) | (%) | (μg/mL) | (%) | (μg/mL) | (%) | (μg/mL) | (%) |

Trastuzumab conjugate

| | | | | | | | | | | |
|---|---|---|---|---|---|---|---|---|---|---|
| DAR2 - XT17* | 0.380 | 47 | >10 | 0 | — | — | — | — | — | — |
| site-specific conjugation | | | | | | | | | | |
| (HC-41C) | | | | | | | | | | |
| DAR2 - XT17* | 0.447 | 41 | >10 | 0 | — | — | — | — | — | — |
| wild-type conjugation | | | | | | | | | | |
| DAR4 - XT17* | 0.176 | 72 | >10 | 0 | — | — | — | — | — | — |
| wild-type conjugation | | | | | | | | | | |
| DAR8 - XT17* | 0.078 | 87 | >10 | 0 | — | — | — | — | — | — |
| wild-type conjugation | | | | | | | | | | |
| DAR8 - XS23* | 0.089 | 91 | >10 | 0 | — | — | 0.076 | 75 | ~1.137^{#} | 80 |
| wild-type conjugation | | | | | | | | | | |
| DAR8 - XT16** | 0.055 | 75 | >10 | 0 | — | — | — | — | — | — |
| wild-type conjugation | | | | | | | | | | |
| DAR8 - XT31/XT32** | 0.140 | 87 | >10 | 0 | — | — | — | — | — | — |
| wild-type conjugation | | | | | | | | | | |
| DAR8 - XT46** | 0.384 | 56 | >10 | 0 | — | — | — | — | — | — |
| wild-type conjugation | | | | | | | | | | |
| DAR8 - XT41** | 0.034 | 92 | >10 | 0 | — | — | — | — | — | — |
| wild-type conjugation | | | | | | | | | | |
| DAR8 - XR4** | 0.051 | 79 | >10 | 0 | — | — | — | — | — | — |
| wild-type conjugation | | | | | | | | | | |
| DAR8 - XT54** | 0.058 | 90 | >10 | 0 | — | — | — | — | — | — |
| wild-type conjugation | | | | | | | | | | |
| DAR8 - XR8-p/XR8-m* | 0.077 | 94 | — | — | ~0.119^{$} | 60 | — | — | — | — |
| wild-type conjugation | | | | | | | | | | |
| DAR8 - XX19* | 0.030 | 94 | — | — | 0.057 | 59 | — | — | — | — |
| wild-type conjugation | | | | | | | | | | |
| DAR8 - XX28* | 0.072 | 88 | — | — | 0.147 | 57 | — | — | — | — |
| wild-type conjugation | | | | | | | | | | |
| DAR8 - XS6* | 0.063 | 86 | — | — | 0.182 | 54 | — | — | — | — |
| wild-type conjugation | | | | | | | | | | |
| DAR8 - XS12* | 0.075 | 91 | — | — | ~0.116^{$} | 57 | — | — | — | — |
| wild-type conjugation | | | | | | | | | | |
| DAR8 - XT89* | 0.075 | 76 | — | — | ~0.301^{$} | 46 | — | — | — | — |
| wild-type conjugation | | | | | | | | | | |
| DAR8 - XR12* | 0.214 | 80 | — | — | 0.289 | 56 | — | — | — | — |
| wild-type conjugation | | | | | | | | | | |
| DAR8 - XR16* | 0.034 | 94 | — | — | 0.128 | 59 | — | — | — | — |
| wild-type conjugation | | | | | | | | | | |
| DAR8 - XX23* | 0.034 | 96 | — | — | 0.072 | 62 | — | — | — | — |
| wild-type conjugation | | | | | | | | | | |
| DAR8 - XT63* | 0.075 | 84 | — | — | 0.148 | 54 | — | — | — | — |
| wild-type conjugation | | | | | | | | | | |
| DAR8 - XT723* | 0.141 | 79 | — | — | — | — | 0.114 | 76 | — | — |
| wild-type conjugation | | | | | | | | | | |
| DAR8 - XT94* | 0.033 | 95 | — | — | — | — | ~0.027^{$} | 93 | — | — |
| wild-type conjugation | | | | | | | | | | |
| DAR8 - XT97* | 0.082 | 84 | — | — | — | — | 0.042 | 85 | — | — |
| wild-type conjugation | | | | | | | | | | |
| DAR8 - XJ9* | 0.052 | 93 | — | — | — | — | 0.032 | 87 | — | — |
| wild-type conjugation | | | | | | | | | | |
| DAR8 - XT100* | 0.045 | 95 | — | — | — | — | 0.024 | 88 | — | — |
| wild-type conjugation | | | | | | | | | | |
| DAR8 - XJ13* | 0.108 | 81 | — | — | — | — | 0.066 | 79 | — | — |
| wild-type conjugation | | | | | | | | | | |
| DAR8 - XJ21* | 0.072 | 87 | — | — | ~0.111^{$} | 57 | — | — | 0.375 | 34 |
| wild-type conjugation | | | | | | | | | | |
| DAR8 - XX23^{a} | 0.043 | 94 | — | — | — | — | 0.027 | 85 | — | — |
| wild-type conjugation | | | | | | | | | | |
| DAR8 - XT114^{a} | 0.010 | 94 | — | — | — | — | 0.008 | 88 | — | — |
| wild-type conjugation | | | | | | | | | | |

*Data fitted with GraphPad Prism software.
**Data fitted with BioAssay software;
^{a}Tested in a later experiment, direct comparison between DAR8-XX23 and DAR8-XT114;
^{#}No full dose-response curve, bottom is missing;
^{$}Steep hillslope;
— Not tested.

TABLE 3b
IC₅₀ values of antifolate ADCs

| | AU-565 cells | | BT-474 cells | | BxPC-3 cells | | FaDu cells | |
|---|---|---|---|---|---|---|---|---|
| | IC₅₀ | Efficacy | IC₅₀ | Efficacy | IC₅₀ | Efficacy | IC₅₀ | Efficacy |
| Compound* | (μg/mL) | (%) | (μg/mL) | (%) | (μg/mL) | (%) | (μg/mL) | (%) |

Trastuzumab conjugate

| | | | | | | | | |
|---|---|---|---|---|---|---|---|---|
| DAR8 - XT123 | 0.010 | 88 | 0.021 | 67 | >10^{#} | 89 | 6.639 | 106 |
| wild-type conjugation | | | | | | | | |
| DAR8 - XT162 | 0.018 | 87 | 0.035 | 67 | >10^{#} | 88 | >10^{#} | 90 |
| wild-type conjugation | | | | | | | | |
| DAR8 - XG2 | 0.015 | 89 | 0.036 | 67 | >10^{#} | 81 | >10^{#} | 37 |
| wild-type conjugation | | | | | | | | |

Sacituzumab conjugate

| | | | | | | | | |
|---|---|---|---|---|---|---|---|---|
| DAR8 - XT123 | 0.022 | 86 | — | — | 0.02 | 82 | 0.019 | 88 |
| wild-type conjugation | | | | | | | | |
| DAR8 - XT162 | 0.024 | 84 | — | — | 0.02 | 83 | 0.018 | 88 |
| wild-type conjugation | | | | | | | | |
| DAR8 - XG2 | 0.020 | 86 | — | — | 0.016 | 80 | 0.016 | 86 |
| wild-type conjugation | | | | | | | | |

*Data fitted with BioAssay software;
^{#}No full dose-response curve, efficacy of these ADCs was determined from the minimum % viability at the highest concentration of the dilution series;
— Not tested.

TABLE 3c
IC₅₀ values of antifolate ADCs

| | Jurkat clone | | Jurkat NucLight | | | |
|---|---|---|---|---|---|---|
| | E6-1 cells | | Red cells | | SK-BR-3 cells | |
| | IC₅₀ | Efficacy | IC₅₀ | Efficacy | IC₅₀ | Efficacy |
| Compound* | (μg/mL) | (%) | (μg/mL) | (%) | (μg/mL) | (%) |

Trastuzumab conjugate

| | | | | | | |
|---|---|---|---|---|---|---|
| DAR8 - XT123 | — | — | >10^{#} | 4 | 0.017 | 95 |
| wild-type | | | | | | |
| conjugation | | | | | | |
| DAR8 - XT162 | — | — | >10^{#} | 0 | 0.027 | 93 |
| wild-type | | | | | | |
| conjugation | | | | | | |
| DAR8 - XG2 | — | — | >10^{#} | 5 | 0.022 | 95 |
| wild-type | | | | | | |
| conjugation | | | | | | |

Sacituzumab conjugate

| | | | | | | |
|---|---|---|---|---|---|---|
| DAR8 - XT123 | — | — | >10^{#} | 9 | 0.026 | 94 |
| wild-type | | | | | | |
| conjugation | | | | | | |
| DAR8 - XT162 | >10^{#} | 0 | >10^{#} | 4 | 0.026 | 90 |
| wild-type | | | | | | |
| conjugation | | | | | | |
| DAR8 - XG2 | — | — | >10^{#} | 0 | 0.021 | 94 |
| wild-type | | | | | | |
| conjugation | | | | | | |

*Data fitted with BioAssay software;
^{#}No full dose-response curve, efficacy of these ADCs was determined from the minimum % viability at the highest concentration of the dilution series;
— Not tested.

TABLE 3d
IC₅₀ values of antifolate ADCs

| | | | | | Jurkat NucLight | |
|---|---|---|---|---|---|---|
| | SK-BR-3 cells | | SW-620 cells | | Red cells | |
| | IC₅₀ | Efficacy | IC₅₀ | Efficacy | IC₅₀ | Efficacy |
| Compound | (μg/mL) | (%) | (μg/mL) | (%) | (μg/mL) | (%) |

825a conjugate

| | | | | | | |
|---|---|---|---|---|---|---|
| DAR10 - XT17** | ~0.375^{#} | 83 | — | — | >10 | 0 |
| wild-type and site-specific | | | | | | |
| conjugation | | | | | | |
| DAR10 - XR16** | >10 | 45 | — | — | >10 | 0 |
| wild-type and site-specific | | | | | | |
| conjugation | | | | | | |
| DAR10 - XX23** | ~0.222^{#} | 76 | — | — | >10 | 0 |
| wild-type and site-specific | | | | | | |
| conjugation | | | | | | |

Control conjugate

| | | | | | | |
|---|---|---|---|---|---|---|
| DAR8 - XT17* | >10 | 8 | >10 | 0 | — | — |
| non-binding control | | | | | | |
| conjugate | | | | | | |

*Data fitted with GraphPad Prism software.
**Data fitted with BioAssay software;
^{#}No full dose-response curve, bottom is missing;
— Not tested.

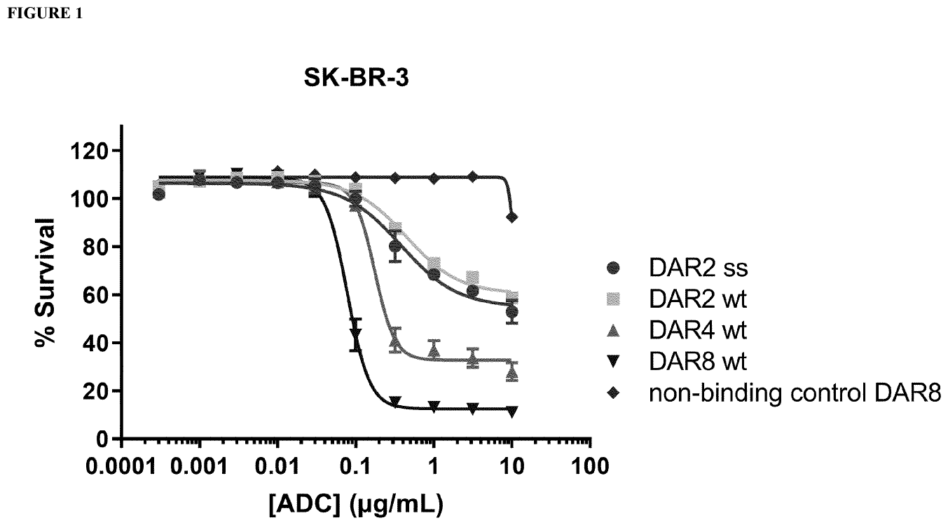

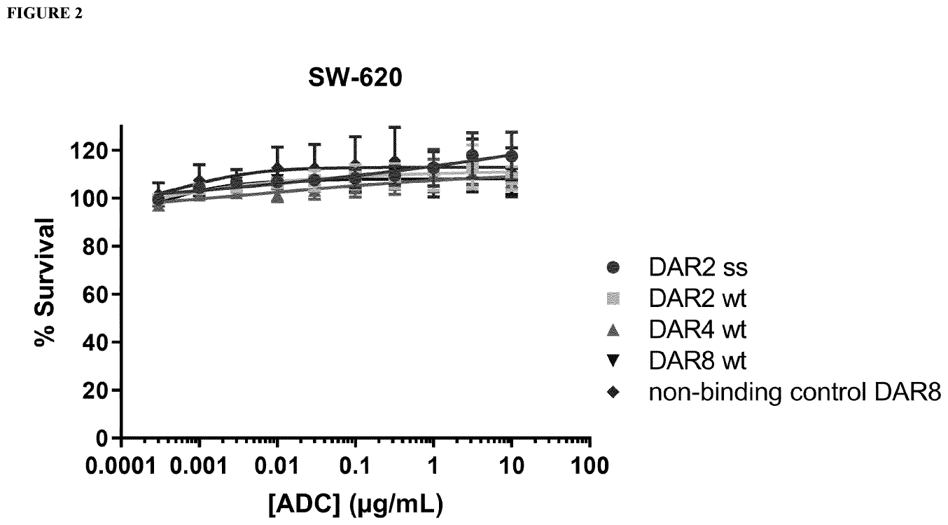

In the HER2-positive SK-BR-3 human tumor cell line, the cytotoxicity of the (site-specific) trastuzumab-XT17 antifolate ADCs increased with DAR (FIG. 1). The cytotoxicity of trastuzumab-XT17 DAR8 and other trastuzumab DAR8-conjugates was mostly shown to be comparable with the exception of trastuzumab-XT46 and trastuzumab-XR12 which appeared somewhat less active, whereas trastuzumab conjugates with XT41, XX19, XR16, XX23, XT94, XT114, XT123, XT162, and XG2 were shown to be more active. The DAR8 sacituzumab conjugates showed similar activity in SK-BR-3 cells compared to their respective corresponding DAR8 trastuzumab conjugates. The DAR10 825a conjugates showed lower activity in SK-BR-3 cells than their respective corresponding DAR8 trastuzumab conjugates. As expected, the non-binding control ADC (rituximab-XT17) had an effect on the growth of the HER2-expressing tumor cells only at high concentrations. All trastuzumab-XT17 antifolate ADCs were inactive (IC₅₀>10 μg/mL) on SW-620, a HER2-negative human tumor cell line (FIG. 2).

### In Vivo Efficacy Experiments

### In Vivo Procedure

The in vivo efficacy of trastuzumab antifolate ADC1 (DAR8-XT17 wild-type conjugated trastuzumab ADC in tables 2 and 3) was evaluated in a BT-474 cell-line (invasive ductal breast carcinoma from a 60-year old Caucasian female patient; Lasfargues et al, J. Nat. Cancer Inst. 1978, 61(4), 967-978) xenograft model in CByJ.Cg-Foxnlnu/J mice and a MAXF574 patient-derived xenograft model (invasive ductal breast carcinoma; triple negative breast cancer) in female NMRI nude mice (Crl:NMRI-Foxnlnu).

### BT-474 Model

Adherent BT-474 cells were grown as monolayer at 37° C. in a humidified atmosphere (5% CO2, 95% air) in Dulbecco's Modified Eagle Medium (DMEM) culture medium containing 4 mM L-glutamine supplemented with 10% FBS. Prior to use, tumor cells were detached from the culture flask by a 5-min treatment with trypsin-EDTA and neutralized by addition of complete culture medium. Cells were counted and viability assessed using a 0.25% trypan blue exclusion assay.

Tumors were induced by subcutaneous injection of 2×10⁷ BT-474 cells in 200 μL of Roswell Park Memorial Institute (RPMI) 1640 medium containing 50% (v/v) matrigel (356237, BD Biosciences, France) into the right flank of healthy female BalB/c Nude ByJ (CByJ.Cg-Foxnlnu/J) mice, 24 to 72 h after a whole body irradiation with a γ-source (2 Gy (Nude mice), ⁶⁰Co, BioMep, France).

Animals were randomized over the treatment groups (n=3/treatment group; mouse clinical trial format) by individual tumor volume when values reached a mean of 150-250 mm³, using Vivo Manager® software (Biosystemes, France). Homogeneity between groups was tested by an analysis of variance (ANOVA). After randomization, the therapeutics were administered by intravenous injection (IV) into the caudal vein.

### MAXF574 Model

Tumor fragments were obtained from xenografts in serial passage in nude mice. After removal from the donor mice, tumors were cut into fragments (3-4 mm edge length) and unilaterally implanted SC in the flank. When tumor implant volumes approached the target range of 80 to 250 mm³, mice were randomized over the treatment groups, aiming at comparable median and mean group tumor volumes. The mice (n=3/treatment group; mouse clinical trial format) were dosed the same day or the following day with a single IV dose injection of 3 or 10 mg/kg antifolate ADC1 into the caudal vein.

Body weights and tumor sizes were measured two or three times a week. The length and width of the tumor were measured with calipers after which the tumor volume was estimated using the following formula: Tumor volume=0.5×length×width² (Simpson-Herren et al, Cancer Chemother. Rep. 1970, 54, 143-174).

### Results

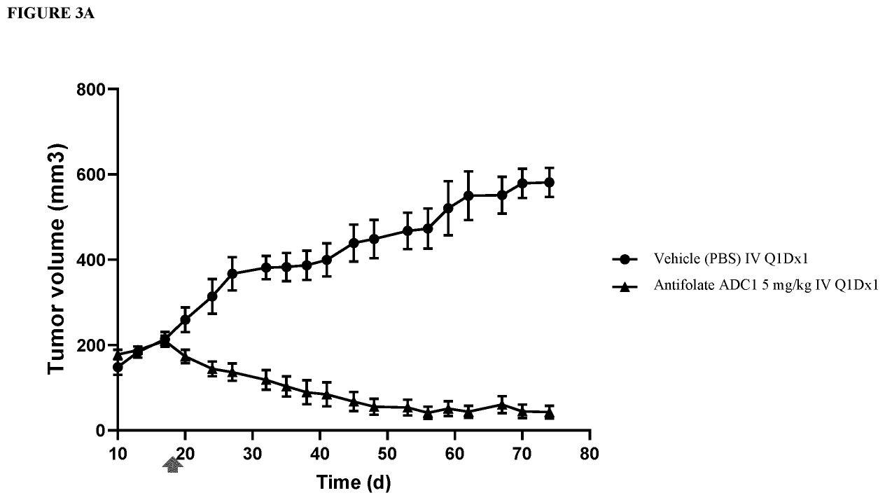

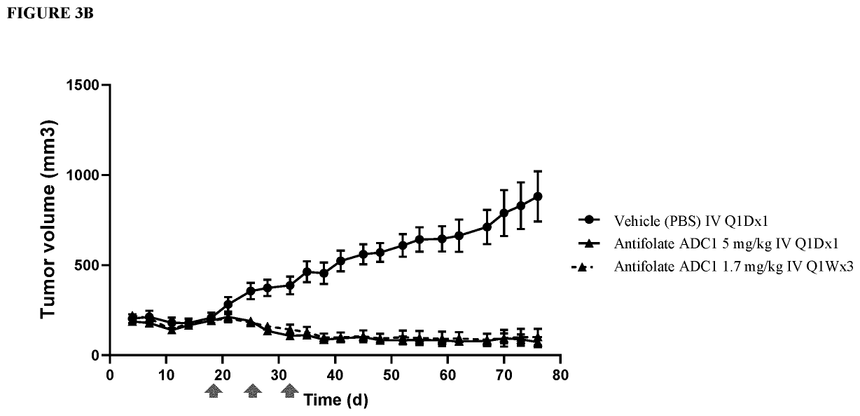

FIG. 3A shows that at a single dose of 5 mg/kg IV, antifolate ADC1 (trastuzumab-XT17; DAR8) reduced the tumor volume in the mouse BT-474 cell line xenograft model. The tumor reducing effect was similar when antifolate ADC1 was given in three separate doses of 1.7 mg/kg IV with one-week intervals (Q1Wx3; FIG. 3B).

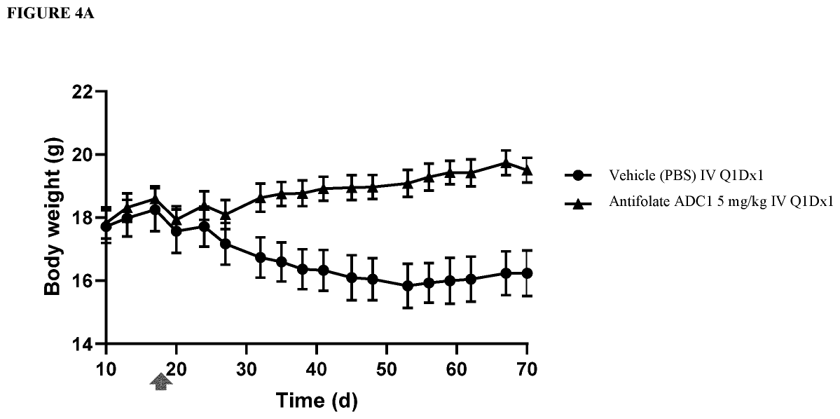

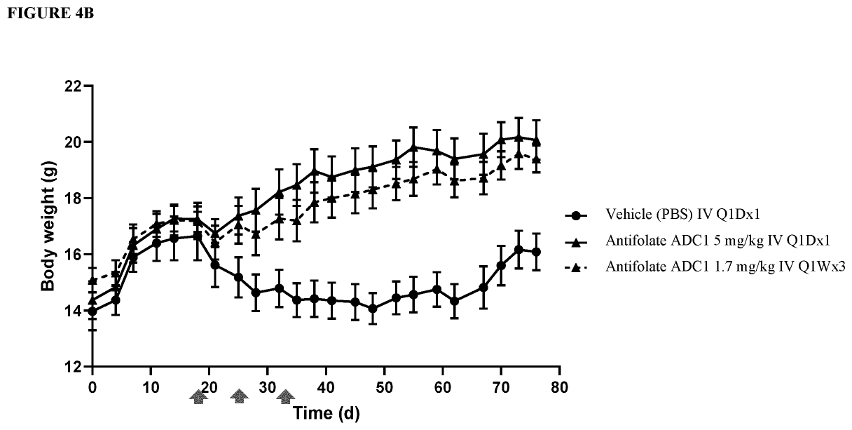

Mice bearing BT-474 tumors develop cachexia as illustrated in FIGS. 4A and 4B. This loss of body weight is often restored after administration of efficacious treatments and is considered a sensitive efficacy biomarker. Treatment with antifolate ADC1 (5 mg/kg IV or 1.7 mg/kg IV Q1Wx3) resulted in a restoration of the body weight (FIG. 4B), similar for both dosing regimens.

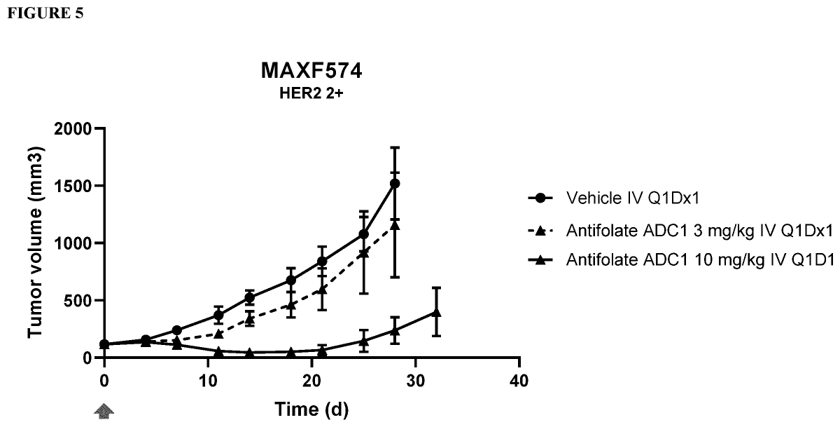

FIG. 5 shows that at a single dose of 3 or 10 mg/kg IV, antifolate ADC1 (trastuzumab-XT17; DAR8) reduced the tumor volume in the mouse MAXF574 patient-derived xenograft model. The tumor reducing effect was dose-dependent and almost complete remission was observed until 20 days after a single IV injection of 10 mg/kg.

1. A linker-drug compound of formula (I) 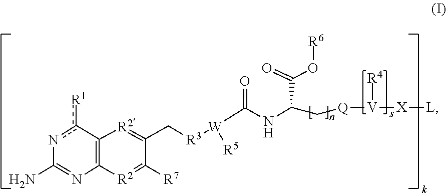
wherein
R¹ is O, NH₂ or OH;
R² and R^{2′} are independently N, CH, CMe, C-Et, C-ethyn, C-vinyl, C—CN, C—OH, C—OMe, C—SH, C—SMe, C-halogen, wherein halogen is preferably Cl, C—CHal₃, C—CHHal₂, or C—CH₂Hal, wherein Hal is halogen, preferably F;
R³ is O, S, NH, N(C₁₋₅ alkyl), N(C₂₋₄ alkenyl), N(C₂₋₄ alkynyl), N—CH₂CN, N—CH₂CH₂Hal, CH₂, CH(C₁₋₅ alkyl), CH(C₂₋₄ alkenyl), CH(C₂₋₄ alkynyl), CH(C₁₋₄ alkoxyl), CH—CH₂CN, or CH—CH₂CH₂Hal, wherein Hal is halogen, preferably F;
R⁴ is H, halogen, —COOH, OH, NH₂, —CONH₂, —CONHR, —CONR₂, C₁₋₄ alkyl, C₁₋₄ alkoxyl, benzyloxy, tetrazole, —SO₃H, —NSO₃H, —OSO₃H, —PO₃H₂, —NPO₃H₂, —OPO₃H₂, —CN, or azido, wherein R is selected from H and C₁₋₅ alkyl, or R⁴ is a carboxylic acid bioisostere selected from the group consisting of 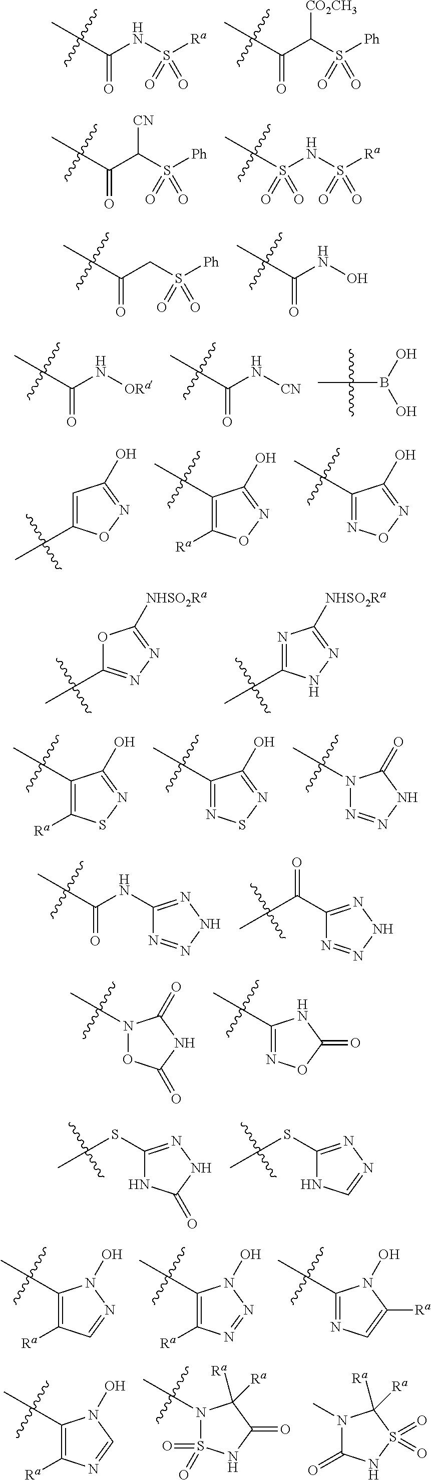 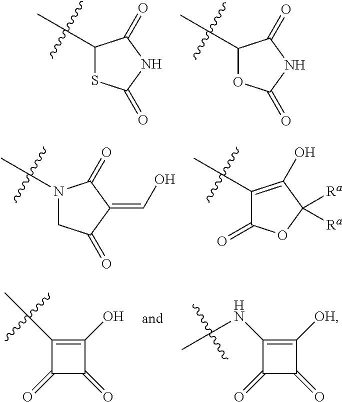
wherein R^{a}′ is selected from H, CH₂F, CHF₂, CF₃, and C₁₋₆ alkyl, each R^{a} is independently selected from H, F, CH₂F, CHF₂, CF₃, and C₁₋₆ alkyl, and two R^{a} substituents can optionally be joined forming a ring;
R⁵ is H, halogen, NH₂, OH, SH, CF₃, C₁₋₄ alkyl, C₂₋₄ alkenyl, C₂₋₄ alkynyl, C₁₋₄ alkoxyl, or C₁₋₄ alkylthio, preferably H, F, CH₃, CF₃, CH₂CH₃, CH═CH₂, CH₂CF₃, or CF₂CF₃, more preferably H or F, or R³ and R⁵ are optionally being joined by one or more bonds to form an optionally substituted carbocycle or heterocycle, preferably an optionally substituted 5- or 6-membered carbocycle or heterocycle;
R⁶ is H, C₁₋₄ alkyl, C₂₋₄ alkenyl, C₃₋₆ cycloalkyl, preferably H;
R⁷ is H or F;
n is 1, 2, 3, or 4, preferably 3;
Q is either absent, —N(R⁸)—(C═O)—, —(C═O)—N(R⁸)—, —CH₂N(R⁸)—, —N(R⁸)CH₂—, —N(R⁸)SO₂—, or —SO₂N(R⁸)—, wherein R⁸ is H, C₁₋₄ alkyl, C₁₋₄ alkenyl, or C₁₋₄ alkynyl, preferably H, or Q is an amide bond bioisostere selected from the group consisting of 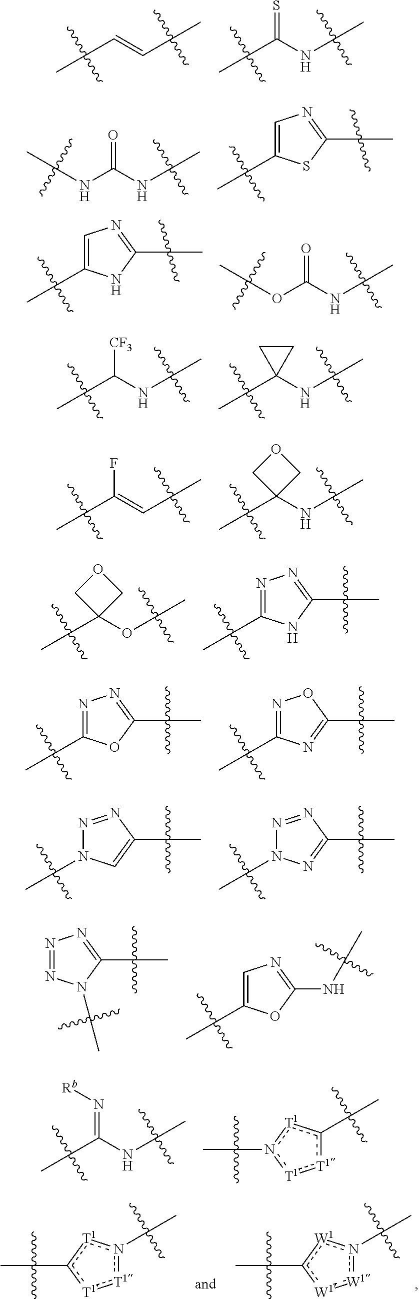
wherein R^{b} is selected from H and C₁₋₅ alkyl, T¹, T^{1′} and T^{1″} are independently selected from CH and N, and W¹, W^{1′} and W^{1″} are independently selected from C, CH, S, N, NH, N(C₁₋₅ alkyl) and O;
V is an aryl, heteroaryl, heterocycle, or cycloalkane that is optionally substituted with one or more R⁴-groups and is independently selected from 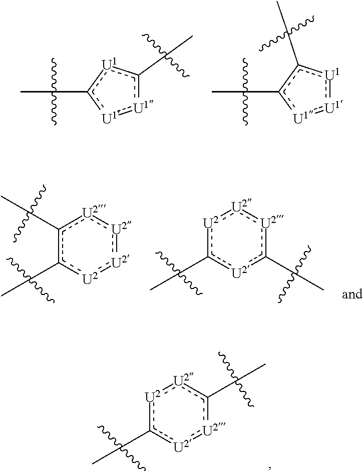
wherein U¹, U^{1′}, U^{1″}, U², U^{2′}, U^{2″} and U^{2′″} are independently selected from C, CH, S, N, NH, N(C₁₋₅ alkyl) and O, or V is selected from the group consisting of 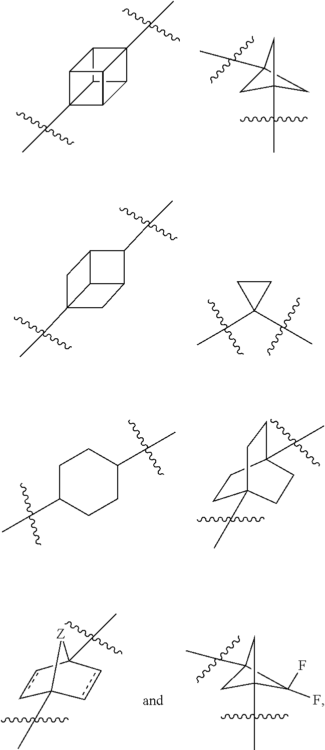
wherein Z is O, S, NH or NR^{c}, and R^{c} is selected from H and C₁₋₅ alkyl;
s is 0 or 1, preferably 1;
W is an aryl or heteroaryl that is optionally substituted with one or more R⁵-groups;
X is a connecting group selected from O, NH, S, C₁₋₅ alkylene, C₁₋₅ alkenylene and C₁₋₅ alkynylene;
k is 1, 2, 3 or 4, preferably 1;
L is a linker moiety; and [symbol]
means that the indicated bond may be a single bond or a non-cumulated, optionally delocalized, double bond.

2. The linker-drug compound according to claim 1 of formula

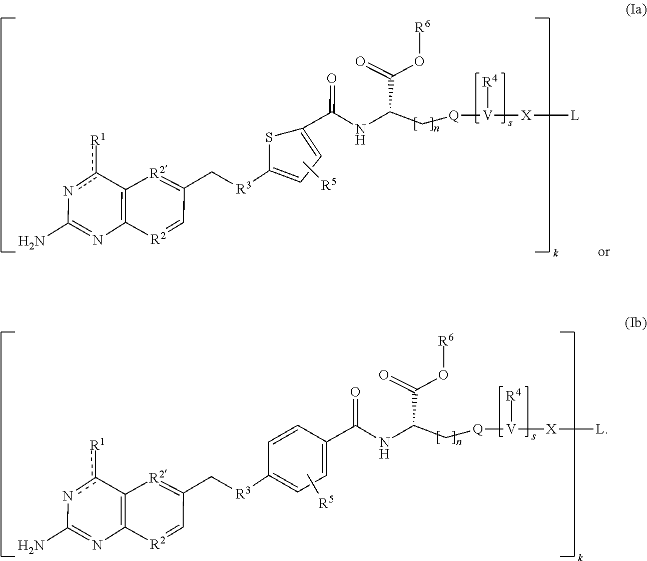

3. The linker-drug compound according to claim 2 of formula

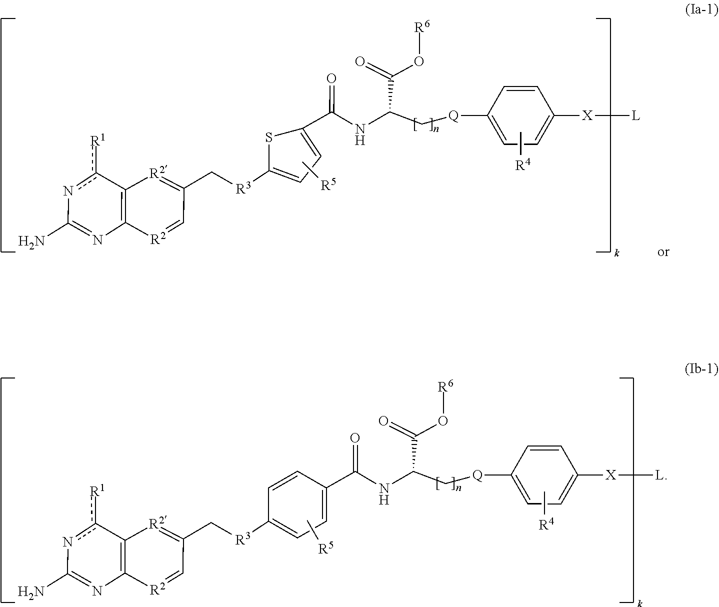

4. The linker-drug compound according to claim 3 of formula

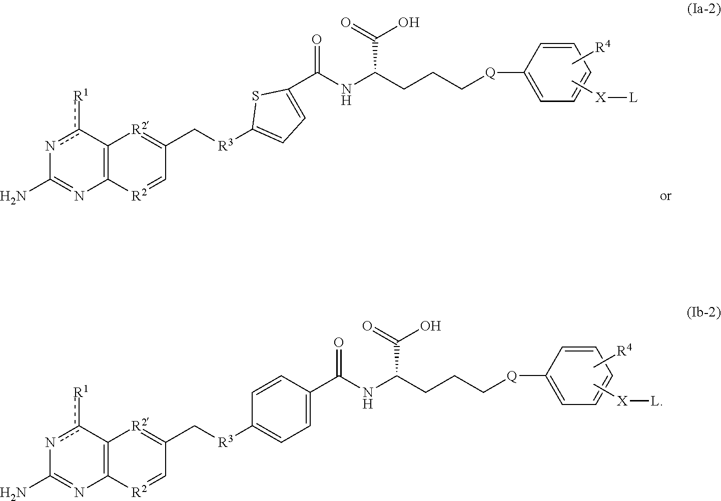

5. The linker-drug compound according to claim 4 of formula

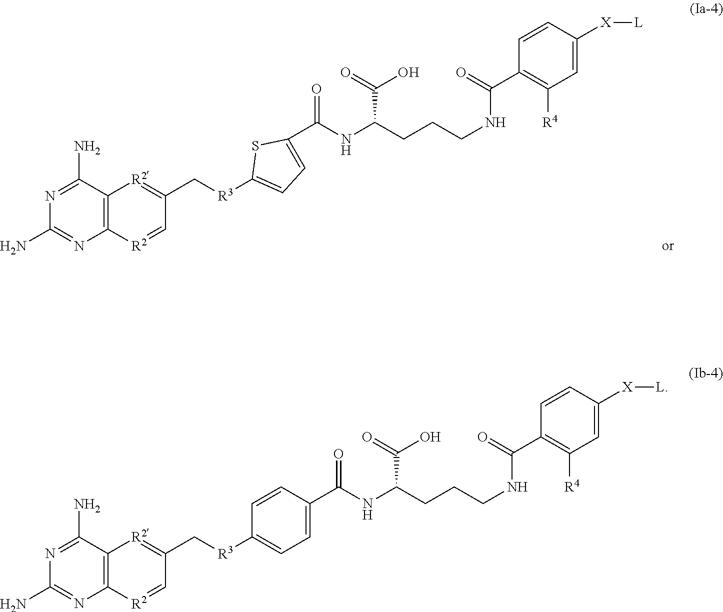

6. The linker-drug compound according to claim 5 of formula

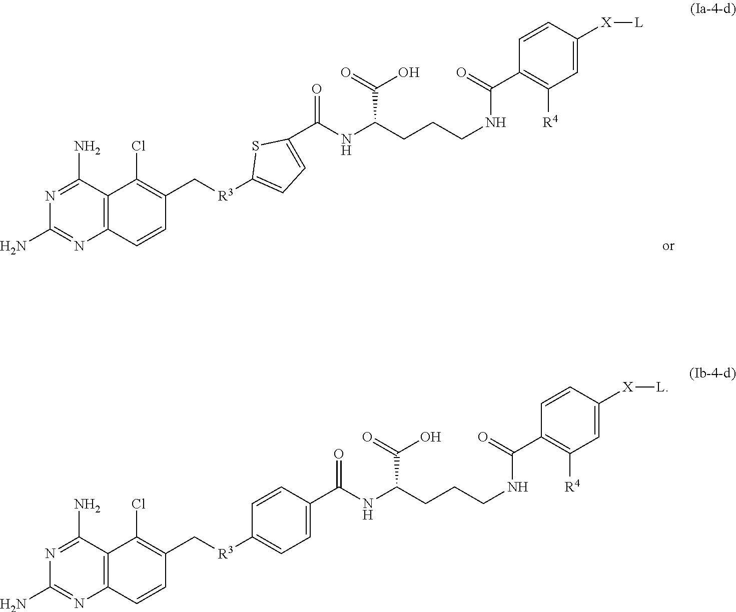

7. The linker-drug compound according to claim 1, wherein L is 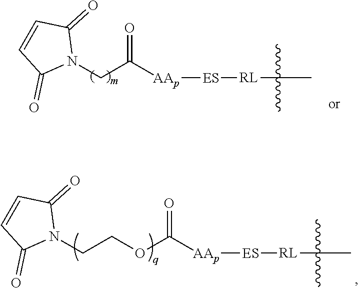
wherein
m is an integer ranging from 1 to 10;
AA is an amino acid;
p is 0, 1, 2, 3, or 4;
q is an integer ranging from 1 to 12;
ES is either absent or an elongation spacer selected from 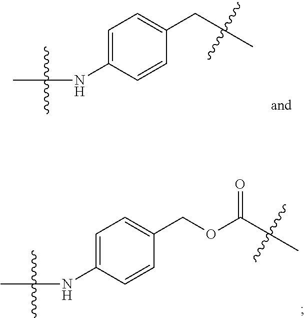 [SMILES: *Cc1ccc(N*)cc1 ; *Nc1ccc(COC(*)=O)cc1]
and
RL is either absent or an elimination spacer selected from 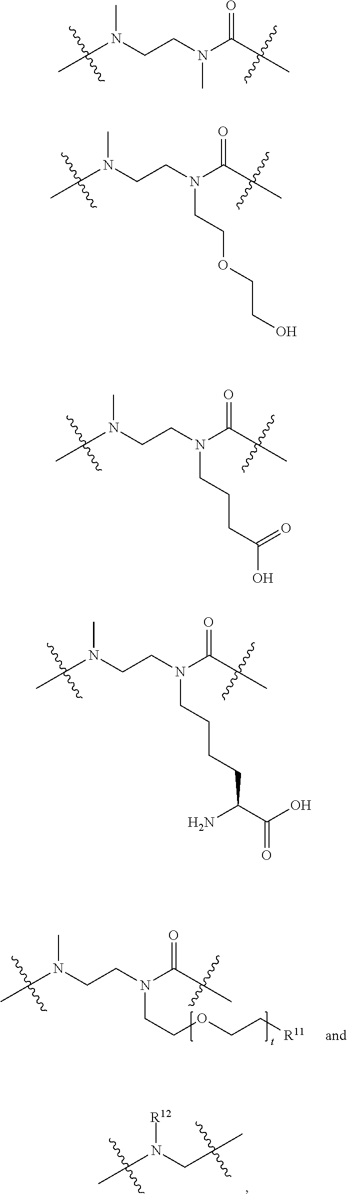
wherein t is an integer ranging from 1-10, R¹¹ is optionally substituted C₁₋₄ alkoxyl, and R¹² is H, optionally substituted C₁₋₆ alkyl, optionally substituted C₆₋₁₄ aryl or optionally substituted C-linked C₃₋₈ heteroaryl

8. The linker-drug compound according to claim 1, wherein L is

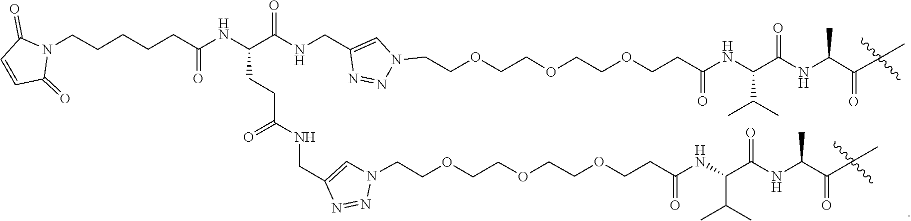 [SMILES: *C(=O)[C@H](C)NC(=O)[C@@H](NC(=O)CCOCCOCCOCCn1cc(CNC(=O)CC[C@H](NC(=O)CCCCCN2C(=O)C=CC2=O)C(=O)NCc2cn(CCOCCOCCOCCC(=O)N[C@H](C(=O)N[C@@H](C)C(*)=O)C(C)C)nn2)nn1)C(C)C]

9. The linker-drug compound according to claim 1, wherein the linker-drug compound is of formula

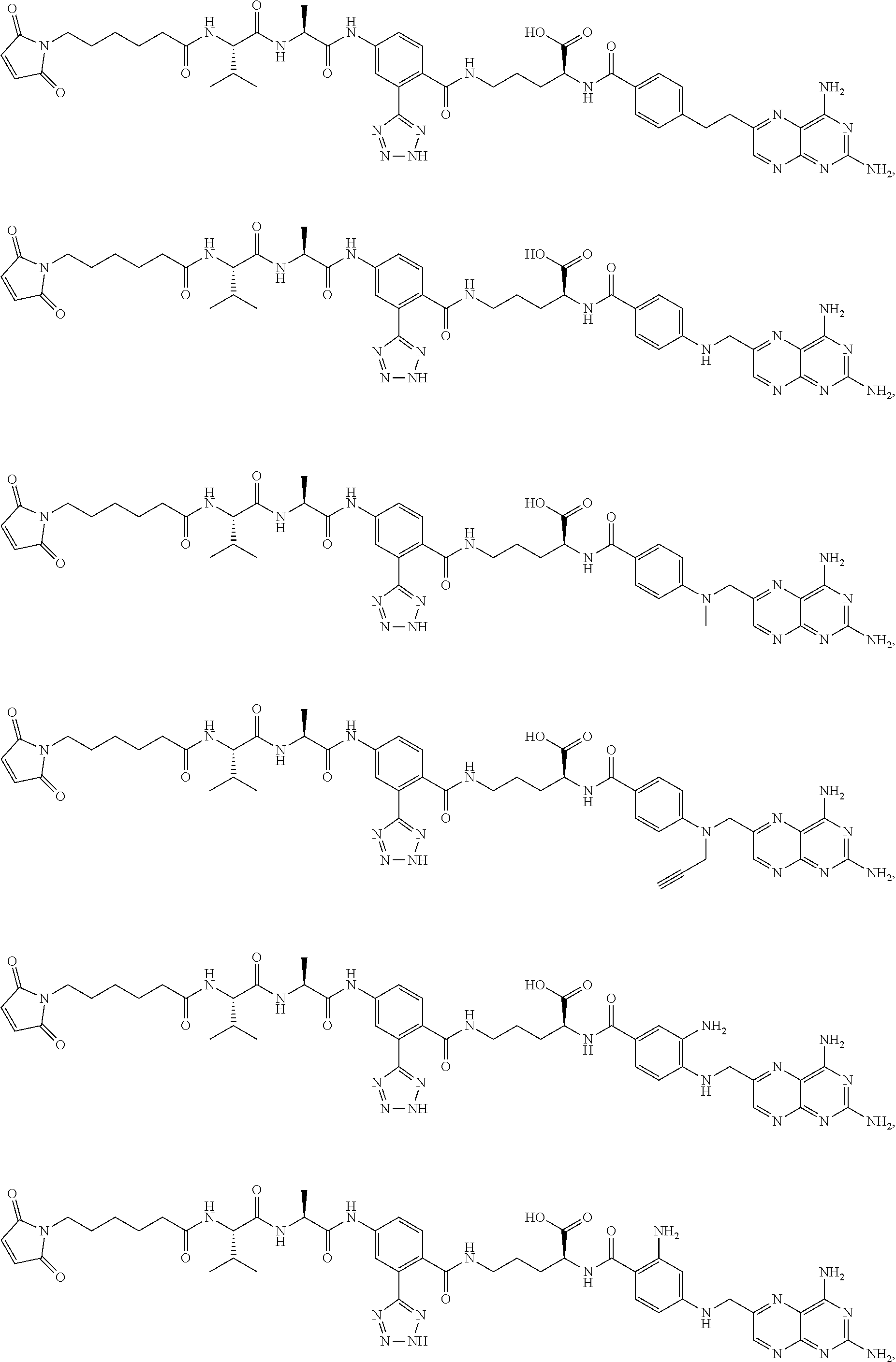

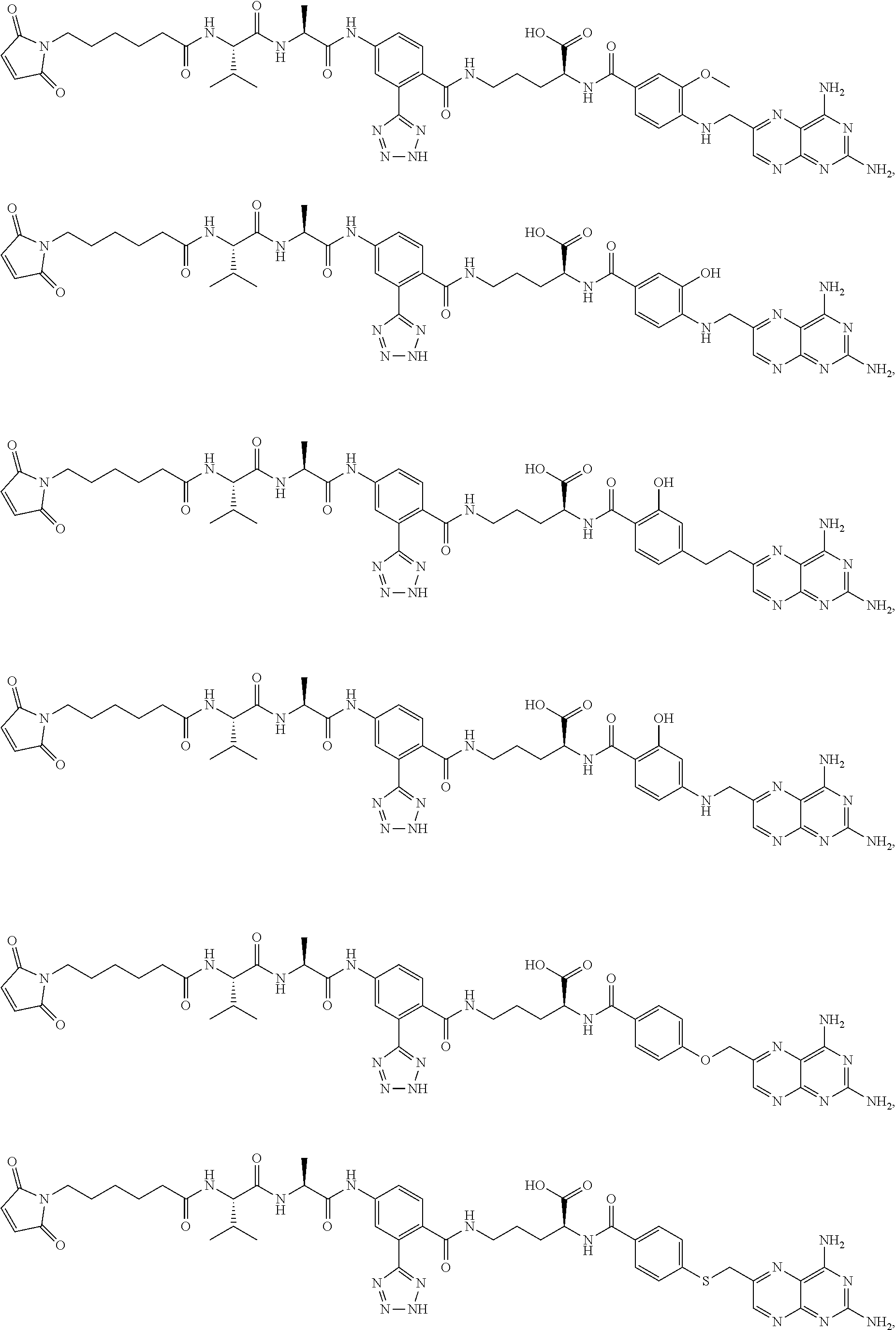

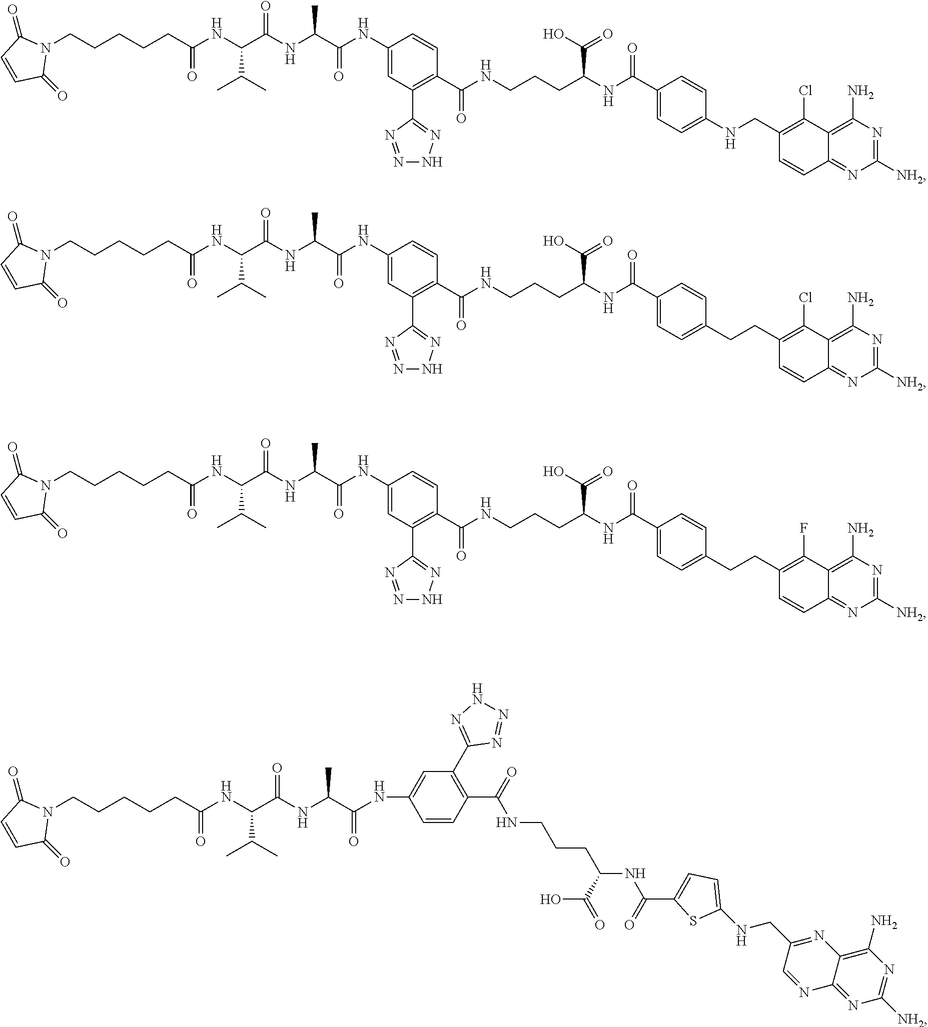

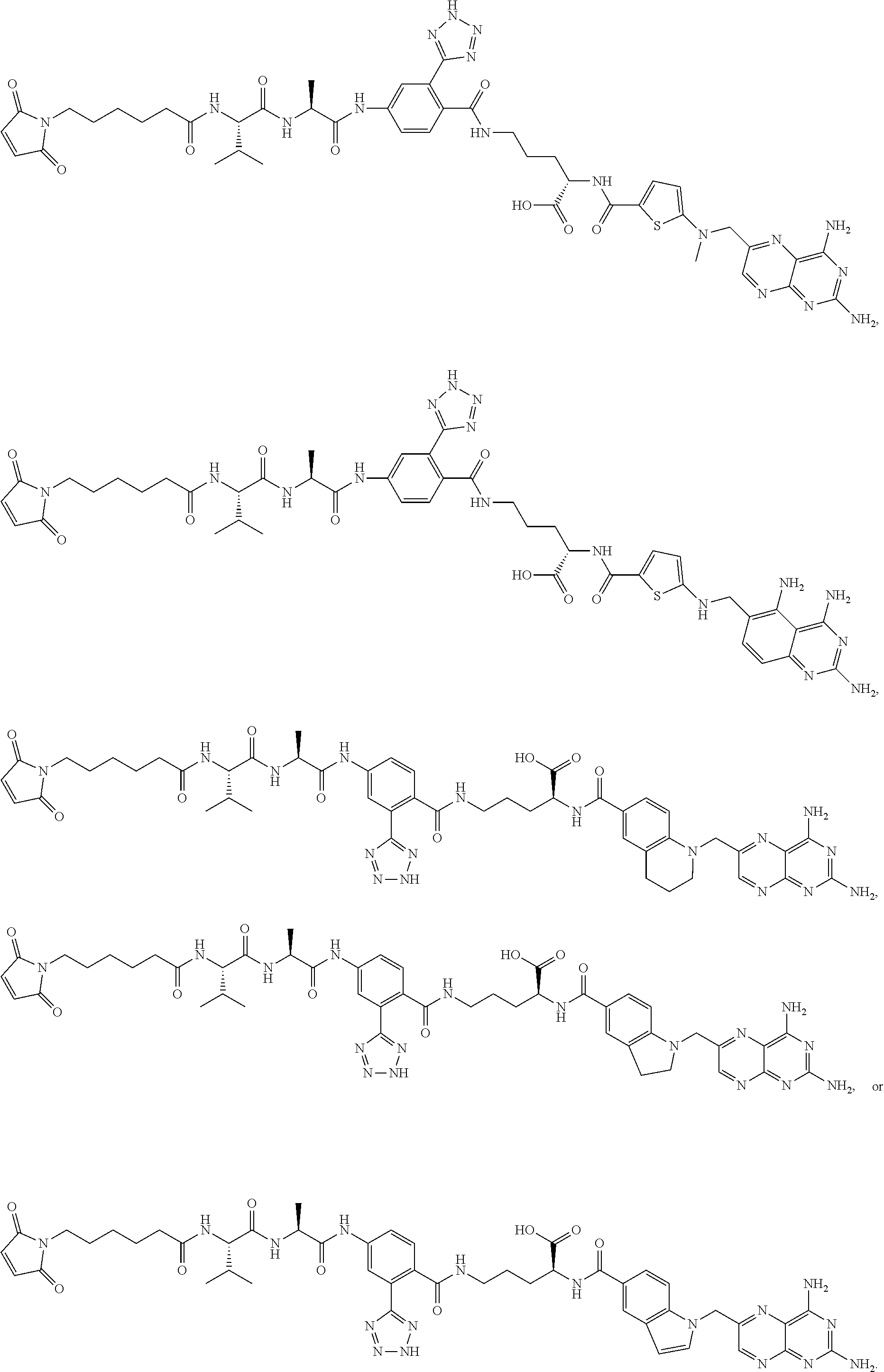

10. The linker-drug compound according to claim 9, wherein the linker-drug compound is of formula

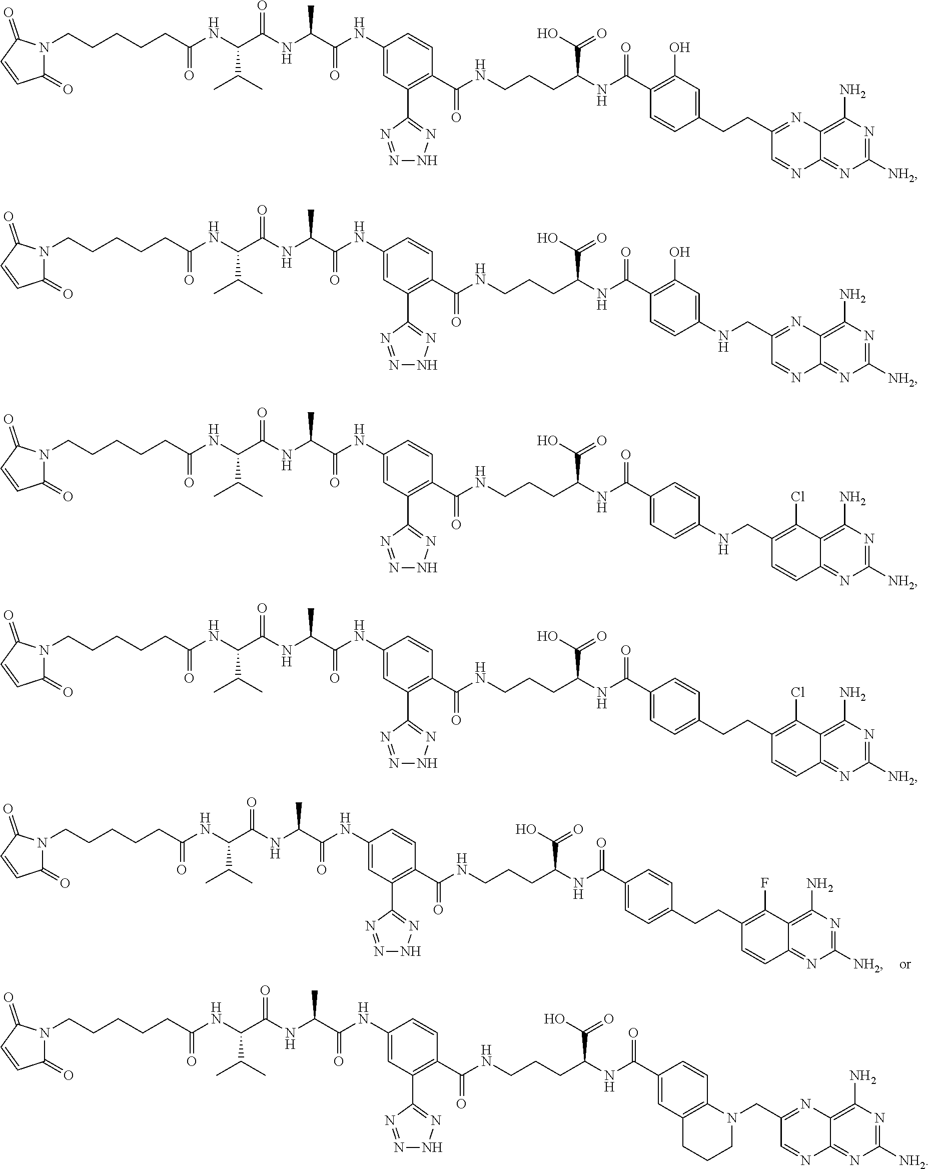

11. An antibody-drug conjugate of formula (III),
Ab-(L-D)_{y} (III),
wherein Ab is an antibody or an antigen-binding fragment thereof;
L-D is a linker-drug compound according to claim 1;
y represents an average drug-to-antibody ratio of from 1 to 16;
and wherein the linker-drug compound is conjugated to the antibody or antigen-binding fragment thereof.

12. The antibody-drug conjugate according to claim 11, wherein the antibody-drug conjugate is of formula 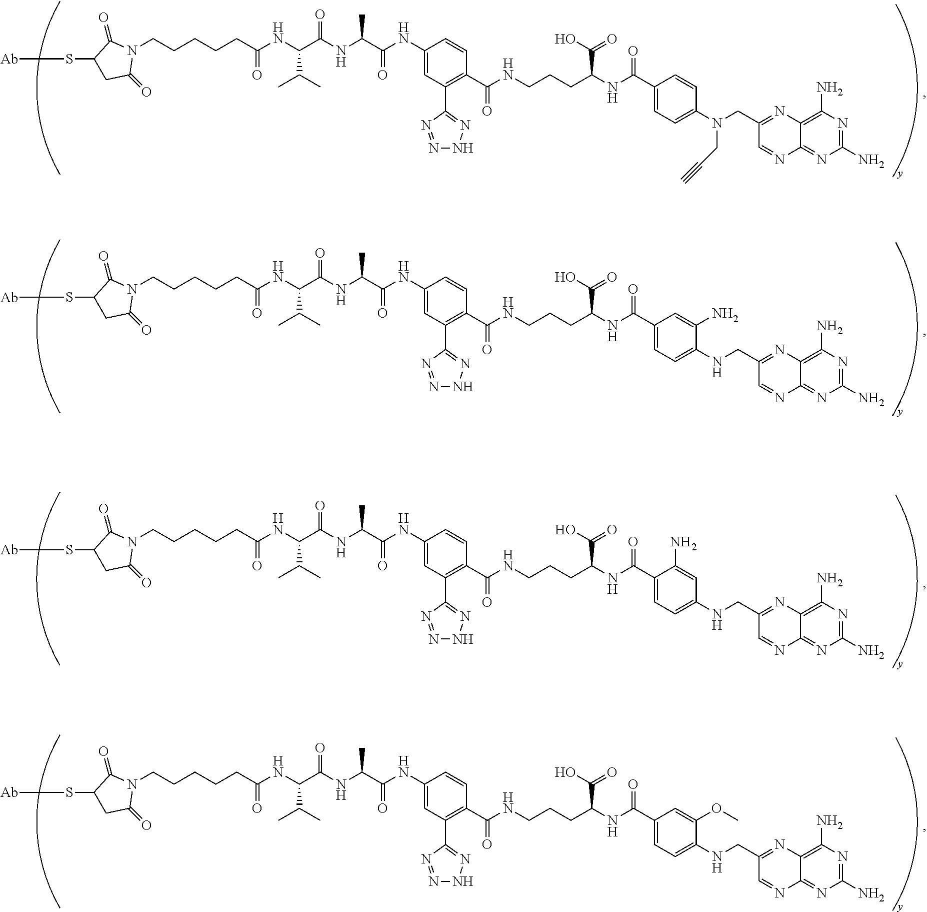 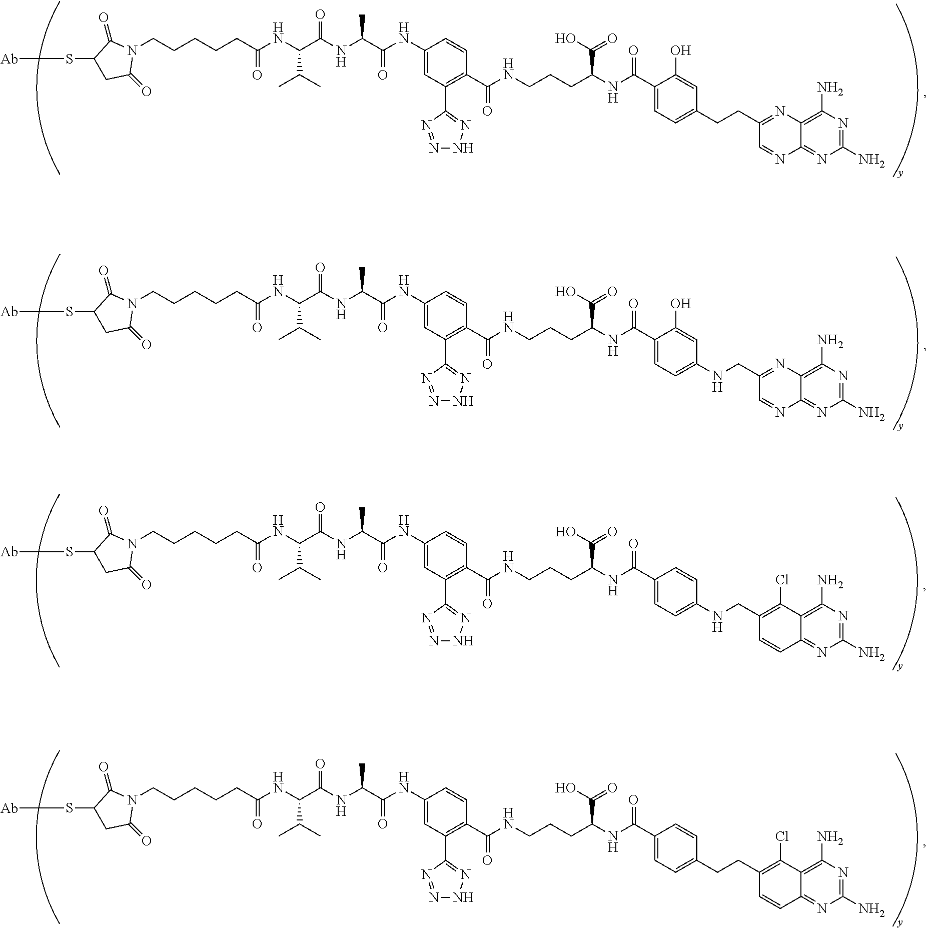 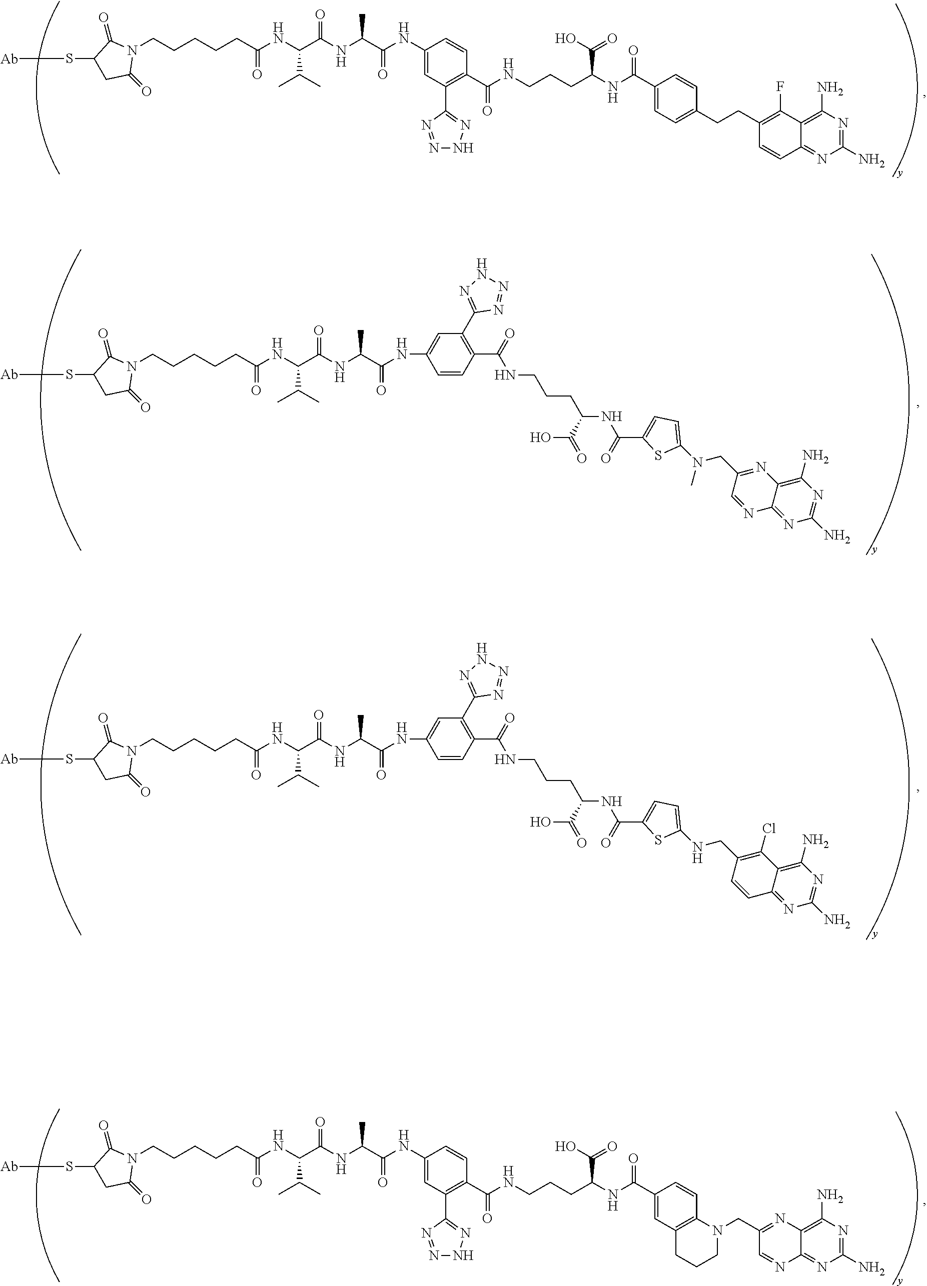 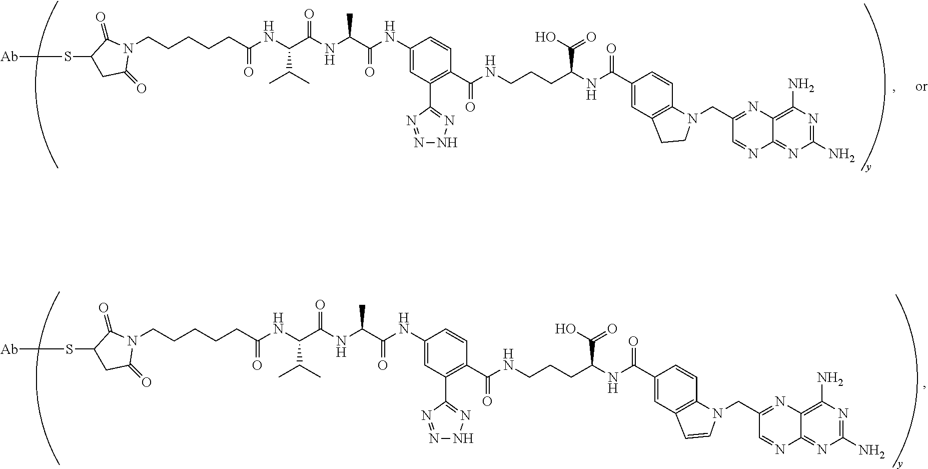
wherein
Ab is an antibody or antigen-binding fragment thereof; and
y represents an average drug-to-antibody ratio of from 1 to 16.

13. The antibody-drug conjugate according to claim 11, wherein the antibody or the antigen-binding fragment binds to an antigen target selected from the group consisting of annexin A1, B7H3, B7H4, BCMA, CA6, CA9, CA15-3, CA19-9, CA27-29, CA125, CA242, CAIX, CCR2, CCR5, CD2, CD19, CD20, CD22, CD24, CD30, CD33, CD37, CD38, CD40, CD44, CD47, CD56, CD70, CD71, CD73, CD74, CD79, CD115, CD123, CD138, CD203c, CD303, CD333, CDCP1, CEA, CEACAM, Claudin 4, Claudin 7, CLCA-1, CLL 1, c-MET, Cripto, DLL3, EGFL, EGFR, EPCAM, EphA2, EPhB3, ETBR, FAP, FcRL5, FGFR3, FOLR1, FRbeta, GCC, GD2, GITR, GLOBO H, GPA33, GPC3, GPNMB, HER2, p95HER2, HER3, HMW-MAA, integrin α, IGF1R, TM4SF1, Lewis A like carbohydrate, Lewis X, Lewis Y, LGR5, LIV1, mesothelin, MN, MUC1, MUC16, NaPi2b, Nectin-4, Notch3, PD-1, PD-L1, PSMA, PTK7, SLC44A4, STEAP-1, 5T4, TF, TF-Ag, Tag72, TNFalpha, TNFR, TROP2, uPAR, VEGFR and VLA.

14. A pharmaceutical composition comprising the antibody-drug conjugate according to claim 11 and one or more pharmaceutically acceptable excipients.

15. (canceled)

16. A method of treating a solid tumor or hematological malignancy in a patient, which comprises administering to a patient in need thereof an effective amount of the antibody-drug conjugate according to claim 11.

17. A method of treating an autoimmune disease in a patient, which comprises administering to a patient in need thereof an effective amount of the antibody-drug conjugate according to claim 11.

18. A method of treating a bacterial, viral, and/or parasitic infection in a patient, which comprises administering to a patient in need thereof an effective amount of the antibody-drug conjugate according to claim 11.

19. (canceled)

20. (canceled)

21. A compound selected from the group consisting of:

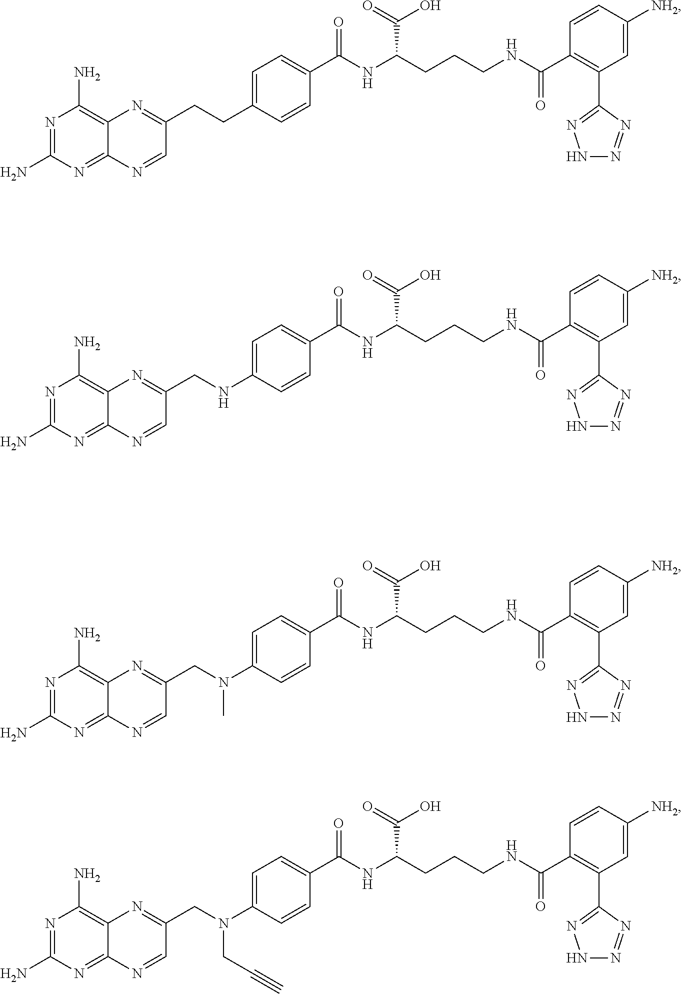 [SMILES: Nc1ccc(C(=O)NCCC[C@H](NC(=O)c2ccc(CCc3cnc4nc(N)nc(N)c4n3)cc2)C(=O)O)c(-c2nn[nH]n2)c1 ; Nc1ccc(C(=O)NCCC[C@H](NC(=O)c2ccc(NCc3cnc4nc(N)nc(N)c4n3)cc2)C(=O)O)c(-c2nn[nH]n2)c1 ; CN(Cc1cnc2nc(N)nc(N)c2n1)c1ccc(C(=O)N[C@@H](CCCNC(=O)c2ccc(N)cc2-c2nn[nH]n2)C(=O)O)cc1 ; C#CCN(Cc1cnc2nc(N)nc(N)c2n1)c1ccc(C(=O)N[C@@H](CCCNC(=O)c2ccc(N)cc2-c2nn[nH]n2)C(=O)O)cc1]

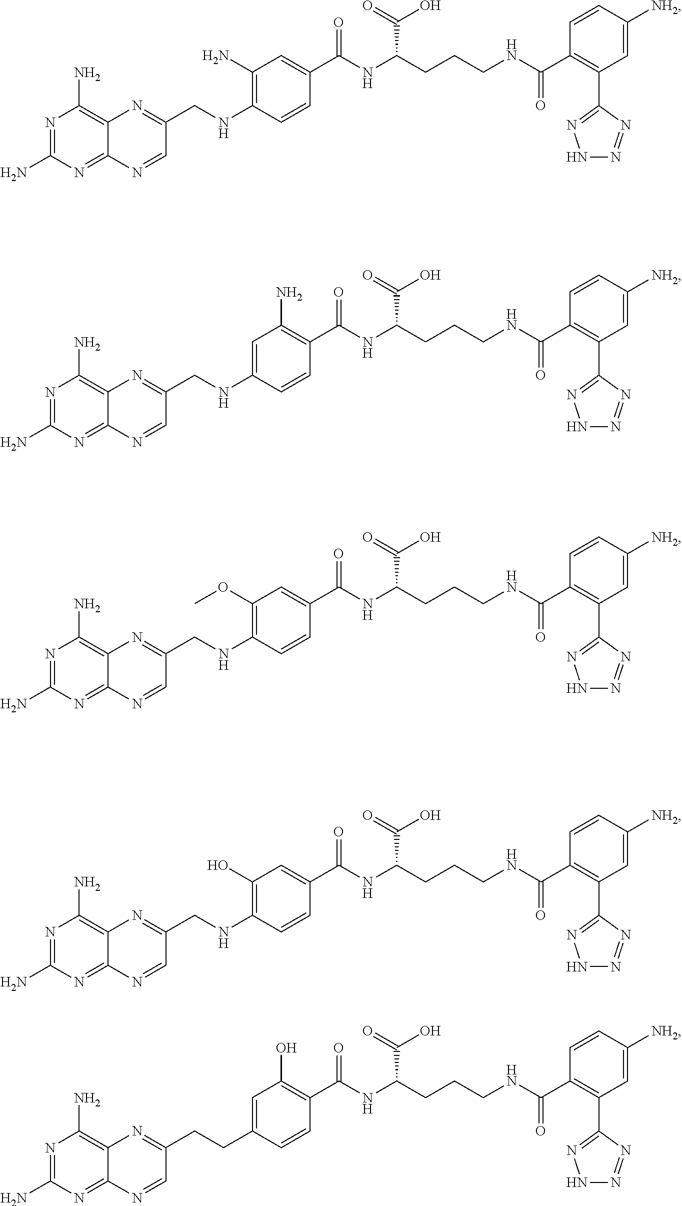 [SMILES: Nc1ccc(C(=O)NCCC[C@H](NC(=O)c2ccc(NCc3cnc4nc(N)nc(N)c4n3)c(N)c2)C(=O)O)c(-c2nn[nH]n2)c1 ; Nc1ccc(C(=O)NCCC[C@H](NC(=O)c2ccc(NCc3cnc4nc(N)nc(N)c4n3)cc2N)C(=O)O)c(-c2nn[nH]n2)c1 ; COc1cc(C(=O)N[C@@H](CCCNC(=O)c2ccc(N)cc2-c2nn[nH]n2)C(=O)O)ccc1NCc1cnc2nc(N)nc(N)c2n1 ; Nc1ccc(C(=O)NCCC[C@H](NC(=O)c2ccc(NCc3cnc4nc(N)nc(N)c4n3)c(O)c2)C(=O)O)c(-c2nn[nH]n2)c1 ; Nc1ccc(C(=O)NCCC[C@H](NC(=O)c2ccc(CCc3cnc4nc(N)nc(N)c4n3)cc2O)C(=O)O)c(-c2nn[nH]n2)c1]

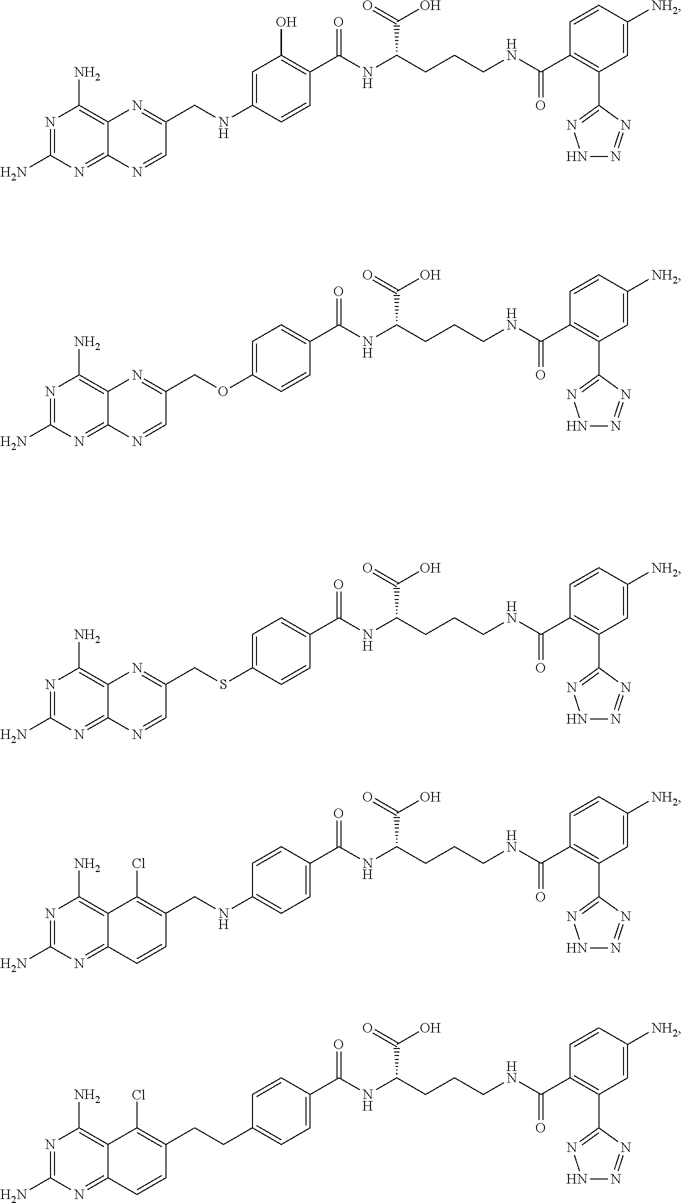 [SMILES: Nc1ccc(C(=O)NCCC[C@H](NC(=O)c2ccc(NCc3cnc4nc(N)nc(N)c4n3)cc2O)C(=O)O)c(-c2nn[nH]n2)c1 ; Nc1ccc(C(=O)NCCC[C@H](NC(=O)c2ccc(OCc3cnc4nc(N)nc(N)c4n3)cc2)C(=O)O)c(-c2nn[nH]n2)c1 ; Nc1ccc(C(=O)NCCC[C@H](NC(=O)c2ccc(SCc3cnc4nc(N)nc(N)c4n3)cc2)C(=O)O)c(-c2nn[nH]n2)c1 ; Nc1ccc(C(=O)NCCC[C@H](NC(=O)c2ccc(NCc3ccc4nc(N)nc(N)c4c3Cl)cc2)C(=O)O)c(-c2nn[nH]n2)c1 ; Nc1ccc(C(=O)NCCC[C@H](NC(=O)c2ccc(CCc3ccc4nc(N)nc(N)c4c3Cl)cc2)C(=O)O)c(-c2nn[nH]n2)c1]

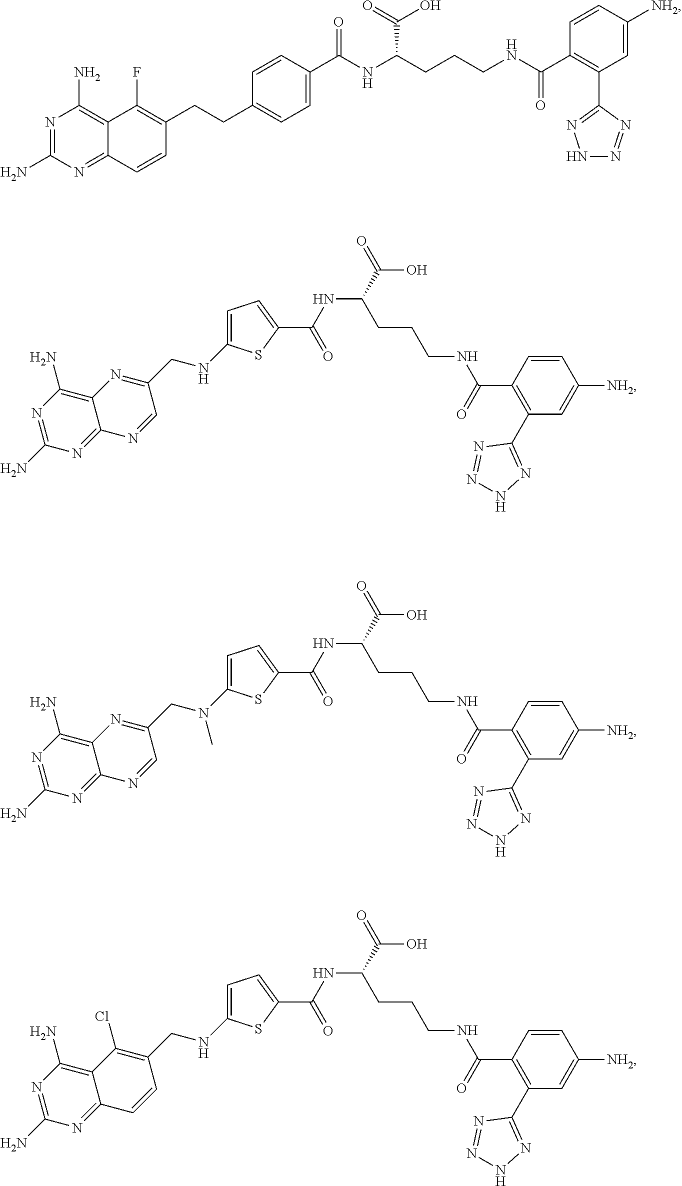 [SMILES: Nc1ccc(C(=O)NCCC[C@H](NC(=O)c2ccc(CCc3ccc4nc(N)nc(N)c4c3F)cc2)C(=O)O)c(-c2nn[nH]n2)c1 ; Nc1ccc(C(=O)NCCC[C@H](NC(=O)c2ccc(NCc3cnc4nc(N)nc(N)c4n3)s2)C(=O)O)c(-c2nn[nH]n2)c1 ; CN(Cc1cnc2nc(N)nc(N)c2n1)c1ccc(C(=O)N[C@@H](CCCNC(=O)c2ccc(N)cc2-c2nn[nH]n2)C(=O)O)s1 ; Nc1ccc(C(=O)NCCC[C@H](NC(=O)c2ccc(NCc3ccc4nc(N)nc(N)c4c3Cl)s2)C(=O)O)c(-c2nn[nH]n2)c1]

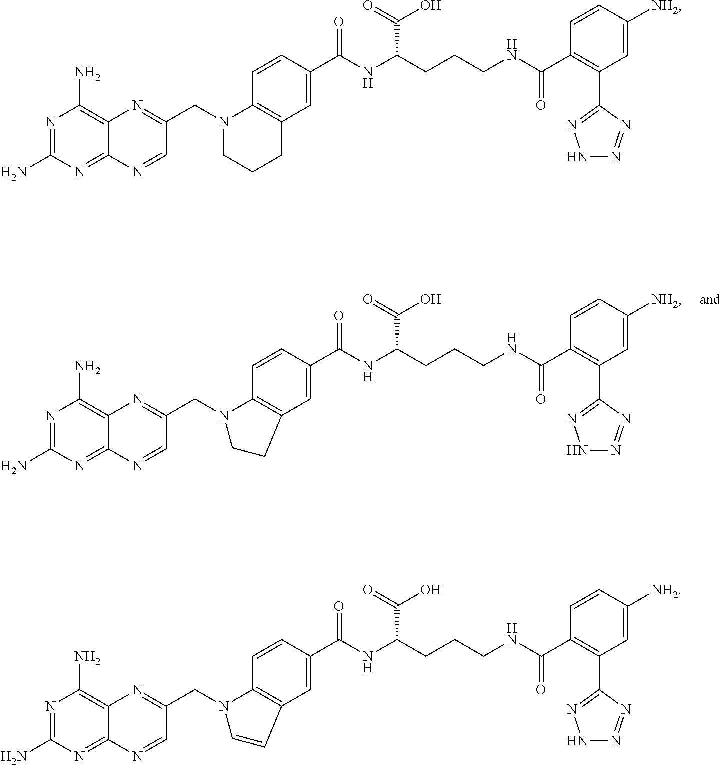 [SMILES: Nc1ccc(C(=O)NCCC[C@H](NC(=O)c2ccc3c(c2)CCCN3Cc2cnc3nc(N)nc(N)c3n2)C(=O)O)c(-c2nn[nH]n2)c1 ; Nc1ccc(C(=O)NCCC[C@H](NC(=O)c2ccc3c(c2)CCN3Cc2cnc3nc(N)nc(N)c3n2)C(=O)O)c(-c2nn[nH]n2)c1 ; Nc1ccc(C(=O)NCCC[C@H](NC(=O)c2ccc3c(ccn3Cc3cnc4nc(N)nc(N)c4n3)c2)C(=O)O)c(-c2nn[nH]n2)c1]

22. The linker-drug compound according to claim 7, wherein L is 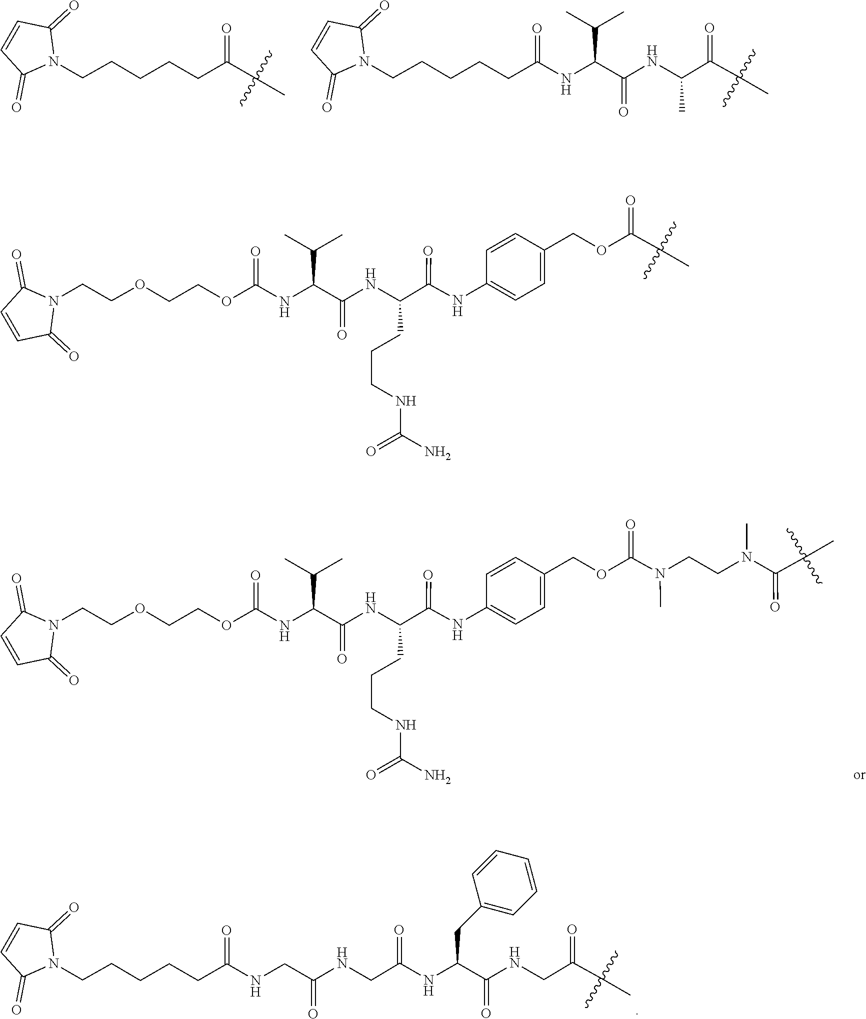 [SMILES: *C(=O)CCCCCN1C(=O)C=CC1=O ; *C(=O)[C@H](C)NC(=O)[C@@H](NC(=O)CCCCCN1C(=O)C=CC1=O)C(C)C ; *C(=O)OCc1ccc(NC(=O)[C@H](CCCNC(N)=O)NC(=O)[C@@H](NC(=O)OCCOCCN2C(=O)C=CC2=O)C(C)C)cc1 ; *C(=O)N(C)CCN(C)C(=O)OCc1ccc(NC(=O)[C@H](CCCNC(N)=O)NC(=O)[C@@H](NC(=O)OCCOCCN2C(=O)C=CC2=O)C(C)C)cc1 ; *C(=O)CNC(=O)[C@H](Cc1ccccc1)NC(=O)CNC(=O)CNC(=O)CCCCCN1C(=O)C=CC1=O]
